SEQUENCE LISTING (774 sequences)

SEQ ID NO: 1 - protein, 944 aa, synthetic construct
MITLGIDYGASNIGIALVLTTEAGENIPLFAGTLRVDARHLKEKVETRAGIRRLRRTRKTKKRRLRNLQHALESLGLSPDQTSKIIRFSKRRGYKSLFDKDTPDETKDDSELTYRFTREEFFKSLEKELSEWISDEVKRAKALSICEKILNRHGNRDHEIRKLRIDNRGVSRCAWEGCRAVTPRLENALKEALSQQLYTVFQTLVRENTAIRNEIDEAVANLTELAKRLRNASGDDANSEKKILRKKARSVLRHLRDRFFALDEPGLEKDKAWKYIESSLMNTLENRGGRNRYCRFHSNEYINTILQGKPVPFKKTIADSDIISRREQIAYAKIWRYIEARLLPLAPEGIDRIVVERTAFDLLAGSWKSIADATDAFKEDMYQQGPMYGFSSRGEMLREEFGGLCAYCGLPSSVLMDRDHILPRADFFFDSYLNIVPACPKCNSALKGRRPASEIALTIHPDAYNAYDHYLRSKFKKRPMHFFHTIKKGILNLMKDPDRLWEAERYLSLITKQFSQIVQTQRSPRPFARYLSSKIGTLQGKTPEIRFRNGRHTALYRNVAYPEFDKYREKAEGNAINHALDAMLLASELPDLYPVESLNIPLSSLKAWSASVKRKAPKPSADGIPSIQNLNDFVDGFEVVHGDGYVDVALRTMVWNQRDTMTHKQDPYGWSAKDKRPTKRTSALDLYTELKKEKAGKVKNKVELIHHPSLRKAVSDAVTPENPGGTAAEALKLWLCNSVRNTLKTSHFSNHPGDRARKEALEKFASNENETIPAVIGVKMFDLGVQGKIDLERLDRQTGNVGHRYMTQPPNRGVIVAYPKASDGHPDMARPCCLYLRQNGAVIPEGLAIFKPLPDKLSKGRIFGSHNNEVSTLFRDVEDYLKDCGFFGYILLTPGCVAHYADGHSWFVRNFDQSKDFKKFRLRNIVAIRKTPFSQKLIPQKVLT

SEQ ID NO: 2 - protein, 960 aa, synthetic construct
MVATTFTVSIDFGSKYIGIALIMHSPAAPNRVLYAAVIVVEAKPLNASINPRTVARRIRRTGKTHRRRLRRLGLALGGIRGAEDVLRFCRRRGYAYDPGEEGDETELAHAVSRDEFFEALAAEVDRVVPERDRGYVLRRCGKHLNAERRGEAELRPARFENRHPSKCQWEGCTRNVPRKGNALREQLAQTLFVWLRPIFDEIGNKGPLRDAVERQIDRMVGLARGYANRPDADEKKALSKQKKRAFADMLDCVADHGDERTVKRFVDNWRKTYSRQLTAILTKKQGGRLRYCRRHSREYVDVFLAGKQPPHRTEVHITDLFGRSQQILFERIWRLVHARILPLANGQIDRVVVERVAFDVLAGPFKQRVKLSEDRAAEMYWHGPMLGFDSRPEMLKEEFDGRCAYCGRKRKLSEVEHILPRSRFPFDSYFNILPSCAECNRGKGARSLLEAGKTVDEKAFEAYSDYIGKKRPPHLFHTIKKGMLKLMTDGGSMATAERQLALLADNLVSITNTQKSPRPLARFLATEIEKLTGHDCKPQWLSGRHTALYREIILPEYDKKEEKENGGLVNHAVDAIVAGCKLPSAAALENPRWYTNQNDGPKQSDMLAWRKKVLSVAPELAGRLPRVEPIERLEFFENDLGDGYVKIDLSAFNWNQQRKSGHKLDPFGTTADGKPLKRKAADDVLKTLLIESKRDGQIAAIAHRGLRQLLERHRKQAARQFVCWLQKTTRKGLADGKRGTHPSDAARYAALEAFVNTPADKFLSNNSATGNETEDKSDEERETIPATIGIRCINAGVKGRLGVTRCNAHHEKGQLLVSHPQYREFYIGYAAGDDGTPDRSRPIVFSVNQAHAVMRRIGSRVEPVTDDPTSVLQGIPLGGWGDRKGFLAQWRAELHAVFEQHGIVRWFRVGQGCYIEKTDGTAFQLCNFDDQKEWMKNGPFKNIQRVYRSPLRGAVERANP

SEQ ID NO: 3 - protein, 942 aa, synthetic construct
MITLGIDYGSSNIGIALVRNTGEGNEPLFAGTIKIDARRLRDKVETRAGIRRLRRTRKTKRHRLRNLRNRFLSLGIKEEDVNLIVGFCKRRGYKSLFDDGETVESEKGDNQLTYRFTREEFFRNLTDELKTILPDSSQFQDALNACEKILNRRGDPFQEIRLIRIDNRGASRCAWEGCDKVTPRRDNALGDPIAQSLLNAVQENLKGEPARMGTIETDISELDTLGRRIRAASGEGAKEEKKALRKNARKVLREIKADFFKPDVDEEERDRAWKYIEAGILNIMENSGGRNRYCREHSKAYIQTVLSGKAAPFKRTISDSDIISRREQIVYQKIWRYLEARVFPLAPEGIDRIVVERTAFDLLAGSRKNIQNASDQFVEEMYQHGPMHGFSSTAEMLKEEFAGMCAYCGCESAALIDRDHILPRADFFFDSYLNIVPACPKCNSDLKGKRPISTSLLRIDDKAYEAYSHYLQKISKSRPMHLFHTIKKGVLNLMRDPERSWEVERYLSLIAKQFSEIVQTQRSPRPFARYLTSKLIKRQEKMPEILFRSGRHINLYRTISYPAFSKVEEKEEGNTANHAIDAMLLASDLPSPSPLEALSIPVSLVKRWSLSVQESAPKTGRNGIPEIPCHGSYVDGFEKVDGNGYVEIELDKMSWNQKDRMTHKQDPYGWSQKAQMPTKRTAAVDLYNSLKKESNVQKVKNIIERIHHPALKRVMAASANSENPGGSVAETMKKWLRQSVQNSINNSSFSNHPGDQARKGDLEKFTSEETAIPAVIGIKMFDTGVRGKIDLSRIDKQTGKVGHRYMTQPANRGVILAYPKKPSGEPDTDRPYLAFIKQDSSLKPEGAMFKPLPEGILNGKILGTGYSPGEWMGQVENYLSECGFHSYVSLTPGCVVCYKNGKKWFVRNFDQSADFKKARLKDVAGIQRTPFITRISPLKMLT

SEQ ID NO: 4 - protein, 938 aa, synthetic construct
MTHTLSIDFGSTYIGLSLLAHNSADPNKVLYAATVVADPAWLTKTVEPRGQIRRMRRTRKTHRRRLHRLAQAMAGIQGATQVLSFCRRRGYSHDSDDDDQDTAAFRVPRAAFFRALLAEVERCVPAADRERVRAACSRHLNEQRTPGAELRPARFDNRGSSRCQWAGCNHNVPRAENCAQEKLSQALFAWLKPVFDGSADPIRLRRSIDHWIGELAGLSRAYRRAAKLEDESKRDENESGIKRRVKRVYKNLKERVCREATPEVAESFSESWNEFYQKNLSEIVYGKRGGRAKYCREHSQQFVEMFLAGQQIPNRQDLSDRDLASLKQQIIFRRLWRLLENRVLPLAGGTIDRLIVERVAIDTLGGLFKDRQQISDKKAGPLYWYGPQYRFNSRLEMLKEEFDGRCAYCGQAANLVDVEHILPHSAFPFDSYLNVVPACDDCNRRKGARTPLQAGMTINENAFDAYVNYVRRQKPPHRLHEIKKGMLKLLLRPGQDGQPERMLGMIANNLVQVTNTQRGPRPLARYLASKLETATGNRPAIAFVSGRHTALYRRVVLPEYDKPAEKESGDVRNHAVDAILLGCQFPSAAALENQTWYRTTADVVTWCNKVRAVSPPLDDGVPEVARHDLVPFFETDLGGGYIGIDLSAFNWNHGRRGTHDLDPFAITRSGKPAKRVPAAKVLAELLLDAGRRDKQIAGIANRGLRNLLEAAPQNAPMAFVTWLQQTTRDGLADGTMGNHPADQARRQQLEKFVAATVENVIADDEPIPAVVGVRCISNISAGLVEVPRCDRSGRVHQHYKANPPVREFYVGYRSKDGAIDGSRPIVFSVNQVYLVRREEGGKKVGLDLPPGSPLLGRPLGAQGRLRDFLVAWRSAFDELCRAEGISRRFRVTQGCVIEKVDGSLFQLRNFDKSEPWRKAASFRNIHRIHRSPLTATDP

SEQ ID NO: 5 - protein, 949 aa, synthetic construct
MITLAIDYGASNIGIALVRNTEEGNEPLFAGTIIIDAKGVKDLAEPRAAIRRLRRTRKTKIRRLRQLSLALKRLGLGEEVSRIVRFARRRGYKSLFDDPDEAKQTKDDVSGYRCTREEFFHELEHELQAIIIDTEQYHRALSVCERVLNRRGERYGEIRPLKIDNRGRSRCAWEGCNRVTPRRHNATDDVVRQQLVTYFQSPLRREPGKLPMLEQAVVKLDIISKNLRGSNEAKCRNALNKRARTILRGLQAALPMHEDEGGSDKESWEYVEKGIVKILENAGGRNRYCREHSKAYCDRVLEGKPSEFKSSIQESDIISRREQIIFNKLWRYIEARLLPLAPEGIDRIVVERTAFDLLAGKQKKIRDASSKSVESIYQYGPRYGFTSEKEMLRKEFGGLCAYCGKPSETLLEREHILPRRLFFFDSYLNILPSCPQCNAAKGTSLPGVSSLRIHEDAYRNYESYINELKSKRPLHFLHTEKKGILNLMRDPERAWDWERYLRLIANNFASIVQTQRGPRPFARYLYSKLSRRQEKPPEIAFRSGRHTALYRSVAYPDFSKAQEKAGEDGNSHPVNHALDALLLASKLPRPEPLEARGLNHASLKAWADQVRGKAAPAGEDGIPVIPAYKCFVPGFEETDAHGYVTVEMAMMNWNKKDTATTKQDPYGWSESRQHPTKRVSARTLFDDLREEKSKKNPSELIKRIYHPALRSAVEKAYQESHNRIQAAEALKNWLRDSVKNSLRSSSFSRHPSDIRRRQDLERFANGKTDEIPVVIGIKCLDTGVAGKIDAARMDRQTGTTGHRYLTDPANRGVVLAYPTTRSGACDRRKPCTAGIRQNYSLKTDETAFRSMPASLERGVVWGKKTHSPRAMESAFSKELEQYLRDRRFHSYCILTAGCVVCYEDGTERFIRNFDKSKGFKKTILRNIVALKRTPLSTRVVPLKVLTALP

SEQ ID NO: 6 - protein, 1007 aa, synthetic construct
MMLPPRLQERLATFLPEIRVGIDFGESAGGIAVVKGNQILHAETYVDFHASDLEQRRQLRRGRRTRHAKKMRLARLRSWVLRQKLPDDTRLPDPYVVMRDPKFHVQPGVFKTKTPGRDSATAPSWIDLAKQGKVNASGFVRALTLIFQKRGFKWDAIELAKMTDEKMKDFLQTARVPSDNLASDIREEIQRRRQDPDSSVRGKKKVSPDELLALLEQARERHPQPRVAEHRTVKEADLRSAVEGFGNSINLTKATIQRWQRELSGLLNKVLRPARFENRLRTGCAWCGKPTPRKIKVRELAYEAAVRNLRVREGRSIRPLKPEELAIFAQWWHLRGQAGESQQGGSEQKRSRKDRSQAVPKLKAIQSHLKKIGAQEQMARQIFDLLWNEKPQGRASLCHQHLIEAAQGRTMKDVVGEWHKVKVRKAPNPCREQHDVRVLHRLEQILFKPGKNGPDAWRYGPAKLITLEVPKPQTEQARKGEQKLRKPESFMERLRKETAGVCMYCDSSTHRPAEDKDHIFPQSRGGPDVWDNLVPVCRDCNMAKGDRTPFEWIGAAGERWQRFTERVEGLAVRGVRVEREDGKEETVRISERKRALLISQDAEYPDNPTPLAHVGARPRQFVVALRKLFEDRGVAAPSVNFESGLPFVQRIDGRTTFQLRKSWLKKANGSDNFPKKNDWDLLNHAQDAALIAACPPHTWRDTVFRVRASRPRWDGKWTEQDGLAVSELAPDWAEYLERQTWPLIKVLGRYPVSWKRKFADLTFSQNPDSLDDKRLVQYLPIANMLHSGKGPDDKRHPAETEIVNPTLDKKFRAVATALGIKRRQTLPEKNLREEFPGIRHVKVRKQPGGRLVRVEPEDGPPRKVEVKGASEAIVFWVKKDEPVTKLRMSIRWPTILRALNVARYEPTIPSDARILAVWRRYQLVRFGPETGLNPGFYRVKEFDAAEVRLLPESAIPDALAQRLNLKRRNDTEESAEENKEIKLRKPALAKYFETLNEKDRHDPRTAS

SEQ ID NO: 7 - protein, 955 aa, synthetic construct
MITLAIDYGASNVGIALVRNTEAGNEPLFAGTVILDARKLKEKVETRAGIRGLRRTRKTKNRRLRELGEALSGLGMEGDKVARIVRFSNRRGYKSLFSDPNETEKVDEAESAYRCTREQFFHQLEQELQEILSDREACDKALSVCERILNRKGDRYAEIRLIRIDNRGASRCAWGDCNKVTPRRDNATDDAIAQQLVTYFQSAIKTEPHKLEMLNQTVCELDSISKNLRGAIANNDDSSKKILRRRARKSLRNLRAELPSTEPEDVSGDAWKYVEKGILNILENSGGRNRYCREHSKSYVEKVLEGKPPEFKSTIADSDIISRREQIAFSKLWRYIEARLLPLAPKGIDRIVVERTAFDLLAGKRKKIRDASSEGVENIYQYGPMYGFPNEKEMLRKEFGGLCAYCGNPSDTLMDRDHILPRRDFFFDSYLNTLPACPTCNSEKSASLPSQVSLRISEDAYSMYKQYLTELASKRPLHFLHTEKKGILNLMRDPARSWEVEKYLSLIANNFASIVQTQRGPRPFARYLYSKLSTRQNKPPKIVFRSGRHTALYRGVAYPLFSKVLEKEQNDLNGKPVNHALDAILLACKLPDSRPLEARGLNLHTLGTWRRAVMAKAPTAGEDGIPTLADQKHYVQGFESTDANGYVTVEMGMMNWNQKDSGTHKQDPYGWSEEKQQPTKRVAAKSLFDELRNDKSKKKPDELIKRIYHPALRAAVEKAFGASQDRLAAAEALKKWLRDSVRNSLSSSSFSRHPSDMRRKQDLEKFAHEDGGDIPIVIGIKCLDKGVEGKIDAERLDKQTGKTGHRYMTDPANRGMILAYPPTPSGEVDKRKPCTAGIRQNYALKTEDTRFASKPPELERGVVWGNNRGSLKDLESKFEKALEKYLGECGFHSYSLLTAGCVVCYEDGTQRFIRNFDKSKDFKKAILKNIDGVRRTPFSKRVLPLKVLTEAPAKE

SEQ ID NO: 8 - protein, 948 aa, synthetic construct
MATTHTLSIDFGSKYIGVALVSHSPKVPNRVLYAAVILVQPKPLNAAIKPRAAIRRIRRTRKTHARRLRRLAQALDGIPGADEVLRFCRRRGYSHDADPKADGEELAYAVSRNEFFAALATEIDRVVPENHREYVLARCQEHLNAERRQTAEIRPARFENRHPTRCQWEGCAKNVARKVNATREQLSQTLFVWLKPAFDQTHKKRPLREAIQRRIDRLVGLARGYGKDPDQETKKVLSKQKRRCFAGILEAVDRFADEETAQQFSENWKKTYSRQFTEILTKKQGGRLRYCRRHSHQFVDLFLAGKQPPHGTEVHMPDLFGRSQQILFQRIWRLVAARILPLAGNRIDRVVVERVAFDILAGPFKQRTEVREDRAAEMYWHGPMYGFGSRREMLKEEFDGRCAYCGRRRSVGEIEHLLPKSRFPFDSYFNILPACRECNQAKGARTPSEVGMTVHQEAYAAFADYVSKKNPPHLYHTIKKGMLKLMTRGGSMATAQRQLGLLADNLVSITNTQKSPRPLARFLADRISRETDRPCQPDWLSGRHTALYREIVLPPEYDKKADKRQNGLVNHAVDAIVAGCKFPSAAALENPRWSHEQKDIHLWREKVLAAATELSGGLPKVEPIERIEFFENDLEHGYLHIDLSAFNWNRQRKSGFKQDPFGATAQGEPLKRKPADEVLANLLSDKDRNGQIESIAHPGLRRLLESRPEEAARLFVDWLQKTTRRGFARAKRGTHPSDKARYAPLEAFSSMPVEDFIRKKTVVGKAKSGSPAERETIPPTIGIRCINKGVRGKLTVTRLNGADGKVQRFAADPQYREWYVGYRDGDDGLPVRTEPMLFAVNQGFAVKRKQGRAWVPVTDDEASILNGVHLGARGDRKLFSSRWRSELESVFQEQGIVKWFRVTQGCYIEKTDGTGFQLRSFDDKKAWMKNGPFENIHRVYRSPLSARR

SEQ ID NO: 9 - protein, 960 aa, synthetic construct
MSRVSATLHDRIREFLPTLRVGIDFGEYTGGIALVRGDAILHAETFLDFHVANLEQRRQLRRGRRTRHARKMRLARLRSWVLRQRANGKRLPDPYCLMRDKQYMVQPGVYRTKGAPPDGSPSWVQLAKDGKVSPAGFVRALSLIFQKRGYKWDAIALEQMSDSKLKEFLESARIPSDDLAADIKKLIERRRLDPNDPIRGKKNRVTPEELERLLETARQREPQPRVAEHRRVKEEDLQAVVEGFGRAANLPEETLERWKRELVGLLNKALRLPRFENRLKSGCSWCGKATPRKGKVRELAYWAAVNNLRVRRWPAPPRPLLVGEIQPFKNWWEDRARAPEQATIKKYLQKIGTQAEMARQFHDLLKNDSPKGRTSLCKQHLEMAARGKTMKDSGVEWQTIYVRKAPNPCREGRDARILHRLEQVLFRRGHTGDAAWRYGPVQFITLEVPEPDTEQAPKGKQKERQEETFLARLLAEFGGKCAYAVLGGCSGEMDKDHVFPRSREGPDVRVNLVPSCKAHNSQKGDRTPFEWLGNDPSRWRAFQESVKGLAIAARKKEILLNETPDYPEGIPTALARIGARPRQFVAALAELFRKYGVAPPRTDYQLGQPLVQRLQGRETDRLRRSWLKKADGSDNFPPKDPTNLSNHAEDAVLLAAAPPHTWRERIFAFRAVRPNWKGEWVEQGGLAAPELAPDWASFQRERESPLIRVLGRYPITWKTSFADQTFGRDPTDLQAPKLRISQPVKTLRVSQIPNIASPFWRDRFKSLADELGLAPRLTIPEDKLAERFPGLRRLQLFRQPGGTLMTVRPEDGPARKIQFKPASEGVVVWQQEVGKKKKRLKTEISIIRPVALQRLGLPRYDPPLPKEGRVIGRLRRHQIIWLDATPKHPPGFYRLTKFQLSGVTAVPENALPQAIARQLRLPRQEAENEVEEELGSITIGKAELASCCAVESRNDGGGSE

SEQ ID NO: 10 - protein, 958 aa, synthetic construct
MSSKLTLSIDFGYKNIGIALVQNNDGTNNPLFAGTLLYDPRQLSDKVEPRAQLRRLRRTRKTKRNRLRKLESRLLSLGLQLEIVQKLITFCRRRGYSSLFDEPQKLDREKKDTKEEIIFPFSREEFFKALEKEIEILLPEDKKSKALIICEAILNRAGDPAQEIRPIRIDNRGASRCAWDGCNNVPPRRDNALRDALSQFIHTVYAAKLRGNNDLTQQVSGMLDRLSVLGKRRRHAGGPDPGKERKILKKAIGEELKLLKSISGFDPELEENDDSAPKTWSSIRRNIVNLIEQSRGRNRFCRQHSSEYVKHILEGKPIPFKHTLSDRDLVSRREQILFQKLWRYIEARILPMAPDGIDRIIVERTAFDLLAGSRKQRQGIARKDALEEMYQQGPRFGFKNDLEMLKMEFDGLCAYCGQQKPEMIEREHLLPQAEFFFDSYLNKVPACKDCNQYLKQAASPGGAGLLIHEQAYEAYSRYLNKKFKDKPPHLFHTVKKGVLNLMRQPDRIWEAERYLNLIANQFAQIVGAQRGPRPLARYLTEKLNKHYGEIPEIAFVNGRHTALWREAAYPHFSKVREKAEGGKINHALDAIIMACDLPDLKALEAKELRPSVIPWWVKRVRNTAPPEGPDGIPVLPKPNNMVSEFEKIHPGNFIEADLSKMNWNHKDSKVQREDSYSWSKKADIPSKRVTAASIIKDLRDADKKDSPESRRNEVKKIIDVVIHPQLGQVLKAANTGDTPGSNTAQALTAWLRKAIGNNFNKTIFSAHPADQRRSKLLQEFVNGQSDGIPAFIGVKILYPWLKTNIDLNRVDPQTNNLLHRYVADPANIGMIVAYKGNHNQVNRDRPITLEWRQSGSVIPGMKSLGQIPDGPLKGRALGEKGISQNEWKEALHQYLANVGIAEYNIVSQGNVAVYKDGSERYIRNFSASYGFKKSLLKGIIGVRRSPFAQKINSNVKIS

SEQ ID NO: 11 - protein, 935 aa, synthetic construct
MPLPSTTIAIDYGAKYIGLALVEHAEGAPNRVLYACTVVVDPKPLKELVKPRADTRRLRRTRKTHRRRLRRLAQSLADVPNADQILRFCGRRGFSHESDDDQDEQTFRVSRARFFQSLEEEVEQAITPEFRERVLAACSKHLNRLRQPSAELRPARFENRGRSRCNWAGCRNNVPRAGHDIQGRLQQCIFLWLQPIFRESNEPERFRKSVDHWVRELAGLAKGHRRSKASTAYRKQINARKRTIYSHVRKRVRREASEETAAQFDSNWSDYYQSNLNNVIEGKDAGRVRYCRQHSAMFVDHIMASEPIPNREDIRDSDLISRTQQIVFRRLWRLIEGRFLPLAGGRIDRVVVERVAFDILSGPIKQRQKMPEDRAAEMYWHGPQAGFDGRRDMLKAEFGNRCAYCGQEAFSEIEHVYNRSDFPFDSYFNIVPACTKCNARKGGRTAFDAGMTIHDDAYAAYCDYLKAKKVLHPYHTIKKGMLNLLRRSATSERAQQLIGMIADNLVSITATQRAPRPLGRYLATKISERTGQRPEIAFRAGRHTALYRSVILPSYDKAEAREADDLRNHAVDAIVLGCRLPSASALENRKWTTSRDDVLRWMESVKEAAPAMQDGLPAVESVVVVPHFETDAGLNYITIALSAFNWNRKRKAAHKLDPFGKTAAGIPMKRMPAATVLANLLKGEKQRDNQIDVIAHRTLRESLLANRDTAGETFVLWLQKSVRAGLTNDEMSSHPADQERRRLLGEFTAAPVSEIVAEPKGSKIPWTIGIRCLNRDTGAPRKVHVARRLGGNDRAQFYPAQAVIREIRVGYREADDQLDRNAPIIFAVNQIDELSRQVPGRWEPVDVPSESPLRGRPLASEGSRKEFRKRWEDAFADLCRKHDIAKVFRITQGCVIEKTDGAKFQIRNFDKSQPWMKGGPFKQIRRVYRSPFQAM

SEQ ID NO: 12 - protein, 946 aa, synthetic construct
MVTLGVDYGASSVGLALVRSDEKGHNIPLFAGTIRLDARWLKEKVEVRAGIRRLRRTKKTKRHRLRQLEQSLTQIGLGAEQIQSIVRFSNRRGYKSLFDAGVADDDHDEAELTYRFTREEFFKALGTELQSIIPDTSQRQKALSTCETILNRQGDRSLEIRQIKIDNRGASRCAWEGCNRVTPRSDNALGEVLSQQIYTVFQSALKVNPVLCQKVETATHELHELAKRLRNASGEAASNEKKILRKRARTILRALKELLYTPAEGVGDAEKAWKYIETGIMNIMESRLGRNRYCREHSREYVQTICSGKLVPFKQTISESDIISRREQIAYAKLWRYIEARILPLTPGGIDRIVVERTAFDLLAGSRKTIQKATDQFKEEMYQHGPMYGFESVPDMLKEEFGGLCAYCGQSSSTLIDVDHILPHADFLFDSYLNILPACPKCNSDLKGDRSVSDASLTIHPDAYRAYSDYLKKKFSTRPMHYFHSIKKGVLNLMQDASRLWEAERYLSLIARQFGQIVQSQRGPRPFARYLSTKLSRRQGQAPAIRFRNGRHTALYRRVAYPDFQKQAEKAEGNVLNHALDAILLASELPDLYPVEALNLPLWQLKNWADTVRARAPKSGDEGVPICPDGPQYVDGFETVHPGGYVEVDLRSMRWNQKDSMTHKQDPYGFSEKTGMPTKRGSAFDLYTKLKKEKNPSKVKSRIALIHHPALRKALSESLQSDTTGSSAAENLKTWLRISVKNSLAHSRFSNHPGDQARRSELEKFITQEDCPIPSVIGVKMFDMGVRGKIDMKRLDRQTGGIGHRYMTQPPNKGVIVAYPKREDGKPDLTKPCCVYHKQDLSMTPENISIFKPPPPILSDGVILGKKPYDKGDRRKALEKYLSDCGFHSYVYLTPGCTIRYKDGNEWFVRNFDSSEDFKKGRLREIIGTRRTPFVDSLIPLKVLSQ

SEQ ID NO: 13 - protein, 1042 aa, synthetic construct
MSIPAQLSAAVQTFLPTLRLGLDLGERAVGIAVVRGNEVLHAETVIDFHEATLKERRRLRRGRRTRRAKKSRIARLRSWILRQIVNGKRLPDPYILMRQKRFQCQPGEYRQKVQLAKSALPSWVEAVKQGRETSDEAFVIALTHLFQKRGYRWGGSDVQAMDDNTLADELRKIRLTPAVAEQVRREVERRKNDPNAPKGFTGKINGIEQLIEQALNRRRQPRVAEHRSIVEDEVRAVVTSFGRHHGIAEDTMTRWRAELVCLLNKPVRAARFENRALTGCTWCGAHTPKKSRPEVQELAYWAAVANVRVAAGRQPRPLTQSERAQFVEWWNADAQRRPTQPAIKRYLTSIGAQEEMARQFADLLNRRNLNGRTNLCLAHLREQAEGAFFCPQHQGVCRSAPNGQHRAVESARSRESSASRVWNPARAWHDRRVVARIERMLFMRDGTPRYGGIPSLITIEVPKPDTAHRYECPHCHEALAVNLRVRYRITKLELKPTKVRQNEAAFTCPQCRKPFEINGKRKIGTPNGLKPINVKLGLTHAVVWWAGGGKKARHVADTNGQCIYCGTNVDVGSVKLDHIFPQSMAGPGIYMNMVAACERCNNEKYNRTPWQWKGHDQAWWQAFEARLDRLFLPMRKREMLLSREASYPENPTALARVGGRAREFMRELQVMFARHAIPSERIVTGYRHDADIVMQMIEGWMTDRLRRSWMSGVDGRENFLPKDRADLRNHAQDAVLVAACPPHTWRERIFCYGPDRDMALPDLAPNWRNYESGMRDRHPLVVPLGRYRIRWRKQFLDQTFWKQPLGRKPVVYRQLAELKKSDASSIKDERIRCAFLSVCQAYNIGSEKTLTEDAQADLQNRLVSLGMVTPVRRVQCFSQKGGLPISVRPHDGPVRITQVKPTSDGVVLWLPAGVRLETARTRDLKISVIRPKPVVGWPSPDVPGQAVSELDPPVPPEAQRIATWYRYQCVRFSPHDGWYRLKEFSEKKLTVMPAIRLPKKLRNDAGGHDGEETGNDEREFGKEALLAAVRANAMATFCDPFD

SEQ ID NO: 14 - DNA, 158 bp, synthetic construct
  source 1..158: mol_type = other DNA
gtttcaatcaaccgatagaaatatcggtccggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 15 - DNA, 184 bp, synthetic construct
  source 1..184: mol_type = other DNA
gttttagtcgaaaggctggagcgttctttggcaattcgaccatgtttccaacaggttgcggctaaaaagagccacgaccgcacctcggcaccctgggatcggacggacagtccccggccctgctgctcggatgcttcatgcgcccgacaaacgacgcggtctatgtggtgagtccgtatttttt

SEQ ID NO: 16 - DNA, 171 bp, synthetic construct
  source 1..171: mol_type = other DNA
gtttcaatcaaactgatttgaaaaaatcagttccggttgaaaagagcatccgtctgaagggcactccgggatagggcagtcccggctcttgctgtttccctggcacatcactgtgctccggaaatgaaccctcggacaagtcgaaagacaggatgtgagcctaatttattt

SEQ ID NO: 17 - DNA, 150 bp, synthetic construct
  source 1..150: mol_type = other DNA
gtttcaggcgacacgaagaaattcgtgtggtgtctgaaaagagccacgtccaggagtccggcacccgtggattggacagtctacggccctgcacggccggtatagagaagccggccaaacggccctggccaatgtggtgagtccattttt

SEQ ID NO: 18 - DNA, 165 bp, synthetic construct
  source 1..165: mol_type = other DNA
gtttcaatcaagctgaagaaattcagctccggttgaaaagagcatccgtctggtagccatgcactccggaatggggcagttccggctcttgcgacttagtgggtgaaagctcattgagccaactacgagacacgtctctacgagacaggatgtgagcccttattt

SEQ ID NO: 19 - DNA, 174 bp, synthetic construct
  source 1..174: mol_type = other DNA
ctttcactctagcgaaagctagagtgaaagaagcccaggcgctgctccagtcctcgccgatgtaacccagcatcggaaccagggtgtgggcatccccgtaggccggtactcggaccccggcaaagggcaagggttggtggcgcactcgacgagcgaggagcgccacctcatttt

SEQ ID NO: 20 - DNA, 159 bp, synthetic construct
  source 1..159: mol_type = other DNA
gtttcaatcaagctgagaaatcagctccggttgaaaagagcatccgtctggcgggcactccggaatggggcagttccggctcttgcagtccagtgagttcattcgcattgaaccaactgccaggcacgtcttcggacaggatgtgagcccgtatttctt

SEQ ID NO: 21 - DNA, 151 bp, synthetic construct
  source 1..151: mol_type = other DNA
gttttggtcagccgaaaggttgcggccaaaaagagccacgaccgctattcggcacccggggatcggacggacagtccccggccctgcggatcgggcgtttcgcacgtccgacaaacgacgcggtccatgtggtgagtccgttttatttttt

SEQ ID NO: 22 - DNA, 192 bp, synthetic construct
  source 1..192: mol_type = other DNA
ctttcactctagcgaaagctagagtgaaagaagcccaggcgcctccgttctcctcgctgtaacccagcagcgggtttaaggcataggaggccccgcttccaggctctcggctcgcctggaactcccgcaagggagaagggcaagggtcgcgacgccgctggccgggcagaaggcgtcgccaaatcgctcttt

SEQ ID NO: 23 - DNA, 157 bp, synthetic construct
  source 1..157: mol_type = other DNA
gtttcagtcaccctgaacgaaagttcgggttcaggctgaaaagagcatctgtccggaagggccactccgggttagggcagatccggcacttgggcctccttggtctttttgactctggaggaaacctttcggcgagtcttcggacaagatgcgagcc

SEQ ID NO: 24 - DNA, 151 bp, synthetic construct
  source 1..151: mol_type = other DNA
gtttcaggcaacatcgaaagatgtcgcgtctgaaaagagcctagacgcgagccagcacccaggaatgcggacagttcctggccctgcatgacggtcatagagaagactgtcgaaatggctctcgtccataatggtgagtccatattatttt

SEQ ID NO: 25 - DNA, 169 bp, synthetic construct
  source 1..169: mol_type = other DNA
gtttcaatcaaactgatgaaaatcagttccggttgaaaagagcatccgtccggagggtgcactccgggatggggcagtcccggcacttgcgttttccccggcttacgcttcggaaaaaggcccttcggcacgtcgaaagacaggatgtgagcccaatttaaatattgtt

SEQ ID NO: 26 - DNA, 226 bp, synthetic construct
  source 1..226: mol_type = other DNA
ctttcagaatgctcgaaagagcattctgaaagaagcccagacgcctcgcgcgcccgcaggtagcccagcttgcggcgtctgtcgtatcggtggggtgagacagacattaaccgcacccacctgcaaataggcgaggcctcaccggggaggttcggcggaccccggcgccactggcgagggcaagggtagagaacgctgcatccgaacctctgcagcgttcgctttt

SEQ ID NO: 27 - protein, 936 aa, synthetic construct
MENFTLAIDFGSVNIGLALVCNRDGVNEPLFAGIITYSNEVLKKKSNPRAQIRRLRRTRKTKKSRLNRLRQALFSLGLDQDVVNALVHFCRRRGYKALYGADREAEENGQAEETVFRYSREEFFRALNKEIDRLLPQDLRPPVLKVCDRVLNRHGELNREIRPIRIDNRGASRCAWSECDKVPPRRKNAIRDALAQFVYAIIDLPRLRNDENLHRDLDAALDTVAELAKRLRSVNGHDPDREKKVLRKRIREALKPVKDLAALDTWKVNSENIMNLLEKSQGRNRYCREHSGEYVRCLLEGRTVPFKASLVERDMVSRREEILYQKLWRYIETRILPLAPGGIDRLVVERVAFDLLAGTRKQRQKVGDKTVEESYQFGPRHGFKSDLEMLRQEFDGLCAYCGKPSGQIIQREHILPRGDFLFDSYLNILPSCPACNQVIKAKASPQAAGLHVHESAYQAYCRYLAGKFKIRPPHEYHTIKKGLLNLMRQPDRTWEAEQYLALIAGHFTQVAQTQRAPRPMARFLCERLRQRFGRSPQLAFRNGRHTDLWRRAAYPDFDKLKEKEEGGLINHALDALVLACDLPGMTALEGLNLKPKELKSWVRAVTVAAPPPGPDGVPVAPEPGRAVPGFEEILPGNYLRADLTFFNWNRKDLGVQSQDIYGWSKNENVPTKRKTALDLVAELRKQTNTSGVKKIIDTVAHPNLRQALQAANTGDKPGEQAVQALIDWLRRAIKPALDKARFSDHPGDQARRKALHDFVSGNLDGPPVTIGVKRLYPYGSGKTDLQRIDPKTGRLVHRYIADPANKALIAAYPLKKGRIRRDSPIILEVRQNDALKPQGKTLARPASGPLAGRGLGRPAPDPDQWKAELHRCLAEAGVAEYAVVAQGCVVKYEDGSEKYIRNFSTSQGFKNGFLKNIVAVRRSPFAAAETANVRLF

SEQ ID NO: 28 - DNA, 183 bp, synthetic construct
  source 1..183: mol_type = other DNA
  misc_difference 1..25: note = a, c, t, g, unknown or other
  misc_difference 1..25: note = This region may encompass 18-25 nucleotides
nnnnnnnnnnnnnnnnnnnnnnnnngtttcagttaccctgaacgaaagttcaggctgaagctgaaaagagcatccgtccggaaggtccactccgggttagggcagatccggctcttggtcctctcctggcccttttcgggctccgagaggaagccttccggcatgtcttcggacaggatgtga

SEQ ID NO: 29 - protein, 7 aa, synthetic construct
PKKKRKV

SEQ ID NO: 30 - protein, 16 aa, synthetic construct
KRPAATKKAGQAKKKK

SEQ ID NO: 31 - protein, 9 aa, synthetic construct
PAAKRVKLD

SEQ ID NO: 32 - protein, 11 aa, synthetic construct
RQRRNELKRSP

SEQ ID NO: 33 - protein, 38 aa, synthetic construct
NQSSNFGPMKGGNFGGRSSGPYGGGGQYFAKPRNQGGY

SEQ ID NO: 34 - protein, 42 aa, synthetic construct
RMRIZFKNKGKDTAELRRRRVEVSVELRKAKKDEQILKRRNV

SEQ ID NO: 35 - protein, 8 aa, synthetic construct
VSRKRPRP

SEQ ID NO: 36 - protein, 8 aa, synthetic construct
PPKKARED

SEQ ID NO: 37 - protein, 8 aa, synthetic construct
PQPKKKPL

SEQ ID NO: 38 - protein, 12 aa, synthetic construct
SALIKKKKKMAP

SEQ ID NO: 39 - protein, 5 aa, synthetic construct
DRLRR

SEQ ID NO: 40 - protein, 7 aa, synthetic construct
PKQKKRK

SEQ ID NO: 41 - protein, 10 aa, synthetic construct
RKLKKKIKKL

SEQ ID NO: 42 - protein, 10 aa, synthetic construct
REKKKFLKRR

SEQ ID NO: 43 - protein, 20 aa, synthetic construct
KRKGDEVDGVDEVAKKKSKK

SEQ ID NO: 44 - protein, 17 aa, synthetic construct
RKCLQAGMNLEARKTKK

SEQ ID NO: 45 - protein, 198 aa, Homo sapiens [sequence omitted]

SEQ ID NO: 46 - protein, 198 aa, Mus musculus
MDSLLMKQKKFLYHFKNVRWAKGRHETYLCYVVKRRDSATSCSLDFGHLRNKSGCHVELLFLRYISDWDLDPGRCYRVTWFTSWSPCYDCARHVAEFLRWNPNLSLRIFTARLYFCEDRKAEPEGLRRLHRAGVQIGIMTFKDYFYCWNTFVENRERTFKAWEGLHENSVRLTRQLRRILLPLYEVDDLRDAFRMLGF

SEQ ID NO: 47 - protein, 198 aa, Canis lupus
MDSLLMKQRKFLYHFKNVRWAKGRHETYLCYVVKRRDSATSFSLDFGHLRNKSGCHVELLFLRYISDWDLDPGRCYRVTWFTSWSPCYDCARHVADFLRGYPNLSLRIFAARLYFCEDRKAEPEGLRRLHRAGVQIAIMTFKDYFYCWNTFVENREKTFKAWEGLHENSVRLSRQLRRILLPLYEVDDLRDAFRTLGL

SEQ ID NO: 48 - protein, 199 aa, Bos taurus
MDSLLKKQRQFLYQFKNVRWAKGRHETYLCYVVKRRDSPTSFSLDFGHLRNKAGCHVELLFLRYISDWDLDPGRCYRVTWFTSWSPCYDCARHVADFLRGYPNLSLRIFTARLYFCDKERKAEPEGLRRLHRAGVQIAIMTFKDYFYCWNTFVENHERTFKAWEGLHENSVRLSRQLRRILLPLYEVDDLRDAFRTLGL

SEQ ID NO: 49 - protein, 239 aa, Rattus sp.
MAVGSKPKAALVGPHWERERIWCFLCSTGLGTQQTGQTSRWLRPAATQDPVSPPRSLLMKQRKFLYHFKNVRWAKGRHETYLCYVVKRRDSATSFSLDFGYLRNKSGCHVELLFLRYISDWDLDPGRCYRVTWFTSWSPCYDCARHVADFLRGNPNLSLRIFTARLTGWGALPAGLMSPARPSDYFYCWNTFVENHERTFKAWEGLHENSVRLSRRLRRILLPLYEVDDLRDAFRTLGL

SEQ ID NO: 50 - protein, 429 aa, Mus musculus
MGPFCLGCSHRKCYSPIRNLISQETFKFHFKNLGYAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQNLCRLVQEGAQVAAMDLYEFKKCWKKFVDNGGRRFRPWKRLLTNFRYQDSKLQEILRPCYIPVPSSSSSTLSNICLTKGLPETRFCVEGRRMDPLSEEEFYSQFYNQRVKHLCYYHRMKPYLCYQLEQFNGQAPLKGCLLSEKGKQHAEILFLDKIRSMELSQVTITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLRRIKESWGLQDLVNDFGNLQLGPPMS

SEQ ID NO: 51 - protein, 429 aa, Rattus sp.
MGPFCLGCSHRKCYSPIRNLISQETFKFHFKNLRYAIDRKDTFLCYEVTRKDCDSPVSLHHGVFKNKDNIHAEICFLYWFHDKVLKVLSPREEFKITWYMSWSPCFECAEQVLRFLATHHNLSLDIFSSRLYNIRDPENQQNLCRLVQEGAQVAAMDLYEFKKCWKKFVDNGGRRFRPWKKLLTNFRYQDSKLQEILRPCYIPVPSSSSSTLSNICLTKGLPETRFCVERRRVHLLSEEEFYSQFYNQRVKHLCYYHGVKPYLCYQLEQFNGQAPLKGCLLSEKGKQHAEILFLDKIRSMELSQVIITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLHRIKESWGLQDLVNDFGNLQLGPPMS

SEQ ID NO: 52 - protein, 370 aa, Macaca mulatta
MVEPMDPRTFVSNFNNRPILSGLNTVWLCCEVKTKDPSGPPLDAKIFQGKVYSKAKYHPEMRFLRWFHKWRQLHHDQEYKVTWYVSWSPCTRCANSVATFLAKDPKVTLTIFVARLYYFWKPDYQQALRILCQKRGGPHATMKIMNYNEFQDCWNKFVDGRGKPFKPRNNLPKHYTLLQATLGELLRHLMDPGTFTSNFNNKPWVSGQHETYLCYKVERLHNDTWVPLNQHRGFLRNQAPNIHGFPKGRHAELCFLDLIPFWKLDGQQYRVTCFTSWSPCFSCAQEMAKFISNNEHVSLCIFAARIYDDQGRYQEGLRALHRDGAKIAMMNYSEFEYCWDTFVDRQGRPFQPWDGLDEHSQALSGRLRAI

SEQ ID NO: 53 - protein, 384 aa, Pan troglodytes
MKPHFRNPVERMYQDTFSDNFYNRPILSHRNTVWLCYEVKTKGPSRPPLDAKIFRGQVYSKLKYHPEMRFFHWFSKWRKLHRDQEYEVTWYISWSPCTKCTRDVATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTSNFNNELWVRGRHETYLCYEVERLHNDTWVLLNQRRGFLCNQAPHKHGFLEGRHAELCFLDVIPFWKLDLHQDYRVTCFTSWSPCFSCAQEMAKFISNNKHVSLCIFAARIYDDQGRCQEGLRTLAKAGAKISIMTYSEFKHCWDTFVDHQGCPFQPWDGLEEHSQALSGRLRAILQNQGN

SEQ ID NO: 54 - protein, 377 aa, Chlorocebus sabaeus
MNPQIRNMVEQMEPDIFVYYFNNRPILSGRNTVWLCYEVKTKDPSGPPLDANIFQGKLYPEAKDHPEMKFLHWFRKWRQLHRDQEYEVTWYVSWSPCTRCANSVATFLAEDPKVTLTIFVARLYYFWKPDYQQALRILCQERGGPHATMKIMNYNEFQHCWNEFVDGQGKPFKPRKNLPKHYTLLHATLGELLRHVMDPGTFTSNFNNKPWVSGQRETYLCYKVERSHNDTWVLLNQHRGFLRNQAPDRHGFPKGRHAELCFLDLIPFWKLDDQQYRVTCFTSWSPCFSCAQKMAKFISNNKHVSLCIFAARIYDDQGRCQEGLRTLHRDGAKIAVMNYSEFEYCWDTFVDRQGRPFQPWDGLDEHSQALSGRLRAI

SEQ ID NO: 55 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 56 - protein, 373 aa, Homo sapiens
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEGQPFMPWYKFDDNYAFLHRTLKEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 57 - protein, 382 aa, Homo sapiens
MNPQIRNPMERMYRDTFYDNFENEPILYGRSYTWLCYEVKIKRGRSNLLWDTGVFRGQVYFKPQYHAEMCFLSWFCGNQLPAYKCFQITWFVSWTPCPDCVAKLAEFLSEHPNVTLTISAARLYYYWERDYRRALCRLSQAGARVTIMDYEEFAYCWENFVYNEGQQFMPWYKFDENYAFLHRTLKEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGN

SEQ ID NO: 58 - protein, 396 aa, Rattus sp.
MQPQGLGPNAGMGPVCLGCSHRRPYSPIRNPLKKLYQQTFYFHFKNVRYAWGRKNNFLCYEVNGMDCALPVPLRQGVFRKQGHIHAELCFIYWFHDKVLRVLSPMEEFKVTWYMSWSPCSKCAEQVARFLAAHRNLSLAIFSSRLYYYLRNPNYQQKLCRLIQEGVHVAAMDLPEFKKCWNKFVDNDGQPFRPWMRLRINFSFYDCKLQEIFSRMNLLREDVFYLQFNNSHRVKPVQNRYYRRKSYLCYQLERANGQEPLKGYLLYKKGEQHVEILFLEKMRSMELSQVRITCYLTWSPCPNCARQLAAFKKDHPDLILRIYTSRLYFYWRKKFQKGLCTLWRSGIHVDVMDLPQFADCWTNFVNPQRPFRPWNELEKNSWRIQRRLRRIKESWGL

SEQ ID NO: 59 - protein, 227 aa, Bos taurus
DGWEVAFRSGTVLKAGVLGVSMTEGWAGSGHPGQGACVWTPGTRNTMNLLREVLFKQQFGNQPRVPAPYYRRKTYLCYQLKQRNDLTLDRGCFRNKKQRHAEIRFIDKINSLDLNPSQSYKIICYITWSPCPNCANELVNFITRNNHLKLEIFASRLYFHWIKSFKMGLQDLQNAGISVAVMTHTEFEDCWEQFVDNQSRPFQPWDKLEQYSASIRRRLQRILTAPI

SEQ ID NO: 60 - protein, 490 aa, Pan troglodytes
MNPQIRNPMEWMYQRTFYYNFENEPILYGRSYTWLCYEVKIRRGHSNLLWDTGVFRGQMYSQPEHHAEMCFLSWFCGNQLSAYKCFQITWFVSWTPCPDCVAKLAKFLAEHPNVTLTISAARLYYYWERDYRRALCRLSQAGARVKIMDDEEFAYCWENFVYNEGQPFMPWYKFDDNYAFLHRTLKEIIRHLMDPDTFTFNFNNDPLVLRRHQTYLCYEVERLDNGTWVLMDQHMGFLCNEAKNLLCGFYGRHAELRFLDLVPSLQLDPAQIYRVTWFISWSPCFSWGCAGQVRAFLQENTHVRLRIFAARIYDYDPLYKEALQMLRDAGAQVSIMTYDEFEYCWDTFVYRQGCPFQPWDGLEEHSQALSGRLRAILQVRASSLCMVPHRPPPPPQSPGPCLPLCSEPPLGSLLPTGRPAPSLPFLLTASFSFPPPASLPPLPSLSLSPGHLPVPSFHSLTSCSIQPPCSSRIRETEGWASVSKEGRDLG

SEQ ID NO: 61 - protein, 190 aa, Homo sapiens
MNPQIRNPMKAMYPGTFYFQFKNLWEANDRNETWLCFTVEGIKRRSVVSWKTGVFRNQVDSETHCHAERCFLSWFCDDILSPNTKYQVTWYTSWSPCPDCAGEVAEFLARHSNVNLTIFTARLYYFQYPCYQEGLRSLSQEGVAVEIMDYEDFKYCWENFVYNDNEPFKPWKGLKTNFRLLKRRLRESLQ

SEQ ID NO: 62 - protein, 190 aa, Gorilla gorilla
MNPQIRNPMKAMYPGTFYFQFKNLWEANDRNETWLCFTVEGIKRRSVVSWKTGVFRNQVDSETHCHAERCFLSWFCDDILSPNTNYQVTWYTSWSPCPECAGEVAEFLARHSNVNLTIFTARLYYFQDTDYQEGLRSLSQEGVAVKIMDYKDFKYCWENFVYNDDEPFKPWKGLKYNFRFLKRRLQEILE

SEQ ID NO: 63 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 64 - protein, 202 aa, Macaca mulatta
MDGSPASRPRHLMDPNTFTFNFNNDLSVRGRHQTYLCYEVERLDNGTWVPMDERRGFLCNKAKNVPCGDYGCHVELRFLCEVPSWQLDPAQTYRVTWFISWSPCFRRGCAGQVRVFLQENKHVRLRIFAARIYDYDPLYQEALRTLRDAGAQVSIMTYEEFKHCWDTFVDRQGRPFQPWDGLDEHSQALSGRLRAILQNQGN

SEQ ID NO: 65 - protein, 185 aa, Bos taurus
MDEYTFTENFNNQGWPSKTYLCYEMERLDGDATIPLDEYKGFVRNKGLDQPEKPCHAELYFLGKIHSWNLDRNQHYRLTCFISWSPCYDCAQKLTTFLKENHHISLHILASRIYTHNRFGCHQSGLCELQAAGARITIMTFEDFKHCWETFVDHKGKPFQPWEGLNVKSQALCTELQAILKTQQN

SEQ ID NO: 66 - protein, 200 aa, Homo sapiens
MALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLGIFASRLYYHWCKPQQKGLRLLCGSQVPVEVMGFPKFADCWENFVDHEKPLSFNPYKMLEELDKNSRAIKRRLERIKIPGVRAQGRYMDILCDAEV

SEQ ID NO: 67 - protein, 210 aa, Macaca mulatta
MALLTAKTFSLQFNNKRRVNKPYYPRKALLCYQLTPQNGSTPTRGHLKNKKKDHAEIRFINKIKSMGLDETQCYQVTCYLTWSPCPSCAGELVDFIKAHRHLNLRIFASRLYYHWRPNYQEGLLLLCGSQVPVEVMGLPEFTDCWENFVDHKEPPSFNPSEKLEELDKNSQAIKRRLERIKSRSVDVLENGLRSLQLGPVTPSSSIRNSR

SEQ ID NO: 68 - protein, 386 aa, Homo sapiens
MNPQIRNPMERMYRDTFYDNFENEPILYGRSYTWLCYEVKIKRGRSNLLWDTGVFRGPVLPKRQSNHRQEVYFRFENHAEMCFLSWFCGNRLPANRRFQITWFVSWNPCLPCVVKVTKFLAEHPNVTLTISAARLYYYRDRDWRWVLLRLHKAGARVKIMDYEDFAYCWENFVCNEGQPFMPWYKFDDNYASLHRTLKEILRNPMEAMYPHIFYFHFKNLLKACGRNESWLCFTMEVTKHHSAVFRKRGVFRNQVDPETHCHAERCFLSWFCDDILSPNTNYEVTWYTSWSPCPECAGEVAEFLARHSNVNLTIFTARLCYFWDTDYQEGLCSLSQEGASVKIMGYKDFVSCWKNFVYSDDEPFKPWKGLQTNFRLLKRRLREILQ

SEQ ID NO: 69 - protein, 236 aa, Homo sapiens
MTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 70 - protein, 229 aa, Mus musculus
MSSETGPVAVDPTLRRRIEPHEFEVFFDPRELRKETCLLYEINWGGRHSVWRHTSQNTSNHVEVNFLEKFTTERYFRPNTRCSITWFLSWSPCGECSRAITEFLSRHPYVTLFIYIARLYHHTDQRNRQGLRDLISSGVTIQIMTEQEYCYCWRNFVNYPPSNEAYWPRYPHLWVKLYVLELYCIILGLPPCLKILRRKQPQLTFFTITLQTCHYQRIPPHLLWATGLK

SEQ ID NO: 71 - protein, 229 aa, Rattus sp. [sequence omitted]

SEQ ID NO: 72 - protein, 224 aa, Homo sapiens
MAQKEEAAVATEAASQNGEDLENLDDPEKLKELIELPPFEIVTGERLPANFFKFQFRNVEYSSGRNKTFLCYVVEAQGKGGQVQASRGYLEDEHAAAHAEEAFFNTILPAFDPALRYNVTWYVSSSPCAACADRIIKTLSKTKNLRLLILVGRLFMWEEPEIQAALKKLKEAGCKLRIMKPQDFEYVWQNFVEQEEGESKAFQPWEDIQENFLYYEEKLADILK

SEQ ID NO: 73 - protein, 224 aa, Mus musculus
MAQKEEAAEAAAPASQNGDDLENLEDPEKLKELIDLPPFEIVTGVRLPVNFFKFQFRNVEYSSGRNKTFLCYVVEVQSKGGQAQATQGYLEDEHAGAHAEEAFFNTILPAFDPALKYNVTWYVSSSPCAACADRILKTLSKTKNLRLLILVSRLFMWEEPEVQAALKKLKEAGCKLRIMKPQDFEYIWQNFVEQEEGESKAFEPWEDIQENFLYYEEKLADILK

SEQ ID NO: 74 - protein, 224 aa, Rattus sp.
MAQKEEAAEAAAPASQNGDDLENLEDPEKLKELIDLPPFEIVTGVRLPVNFFKFQFRNVEYSSGRNKTFLCYVVEAQSKGGQVQATQGYLEDEHAGAHAEEAFFNTILPAFDPALKYNVTWYVSSSPCAACADRILKTLSKTKNLRLLILVSRLFMWEEPEVQAALKKLKEAGCKLRIMKPQDFEYLWQNFVEQEEGESKAFEPWEDIQENFLYYEEKLADILK

SEQ ID NO: 75 - protein, 224 aa, Bos taurus
MAQKEEAAAAAEPASQNGEEVENLEDPEKLKELIELPPFEIVTGERLPAHYFKFQFRNVEYSSGRNKTFLCYVVEAQSKGGQVQASRGYLEDEHATNHAEEAFFNSIMPTFDPALRYMVTWYVSSSPCAACADRIVKTLNKTKNLRLLILVGRLFMWEEPEIQAALRKLKEAGCRLRIMKPQDFEYIWQNFVEQEEGESKAFEPWEDIQENFLYYEEKLADILK

SEQ ID NO: 76 - protein, 224 aa, Petromyzon marinus; identical to SEQ ID NO: 75

SEQ ID NO: 77 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYRRQGRCQEGLRTLAEAGAKISIMTYSEFKHCWDTFVDHQGCPFQPWDGLDEHSQDLSGRLRAILQNQEN

SEQ ID NO: 78 - protein, 184 aa, Homo sapiens
MDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQ

SEQ ID NO: 79 - protein, 184 aa, Homo sapiens
MDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYRRQGRCQEGLRTLAEAGAKISIMTYSEFKHCWDTFVDHQGCPFQPWDGLDEHSQDLSGRLRAILQ

SEQ ID NO: 80 - protein, 101 aa, Bacillus subtilis
EACKALGTWRLEGATLYVTLEPCPMCAGAVVLSRVEKVVFGAFDPKGGCSGTLMNLLQEERFNHQAEVVSGVLEEECGGMLSAFFRELRKKKKAARKNLSE

SEQ ID NO: 81 - protein, 161 aa, Bacillus subtilis
MTQDELYMKEAIKEAKKAEEKGEVPIGAVLVINGEIIARAHNLRETEQRSIAHAEMLVIDEACKALGTWRLEGATLYVTLEPCPMCAGAVVLSRVEKVVFGAFDPKGGCSGTLMNLLQEERFNHQAEVVSGVLEEECGGMLSAFFRELRKKKKAARKNLSE

SEQ ID NO: 82 - protein, 167 aa, Escherichia coli
MSEVEFSHEYWMRHALTLAKRAWDEREVPVGAVLVHNNRVIGEGWNRPIGRHDPTAHAAIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 83 - protein, 167 aa, synthetic construct
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 84 - protein, 167 aa, synthetic construct
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGWRNAKTGAAGSLMDVLHYPGMNHRVEITEGILADECAALLCYFFRMPRQVFNAQKKAQSSTD

SEQ ID NO: 85 - protein, 167 aa, synthetic construct
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRWAKTGAAGSLMDVLHYPGMNHRVEITEGILADECAALLCYFFRMPRQVFNAQKKAQSSTD

SEQ ID NO: 86 - protein, 167 aa, synthetic construct
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRQAKTGAAGSLMDVLHYPGMNHRVEITEGILADECAALLCYFFRMPRQVFNAQKKAQSSTD

SEQ ID NO: 87 - protein, 167 aa, synthetic construct
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGFRNAKTGAAGSLMDVLHYPGMNHRVEITEGILADECAALLCYFFRMPRQVFNAQKKAQSSTD

SEQ ID NO: 88 - protein, 167 aa, synthetic construct
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGQRNAKTGAAGSLMDVLHYPGMNHRVEITEGILADECAALLCYFFRMPRQVFNAQKKAQSSTD

SEQ ID NO: 89 - protein, 167 aa, synthetic construct
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGMRNAKTGAAGSLMDVLHYPGMNHRVEITEGILADECAALLCYFFRMPRQVFNAQKKAQSSTD

SEQ ID NO: 90 - protein, 167 aa, synthetic construct
MSEVEFSHEYWMRHALTLAKRARDEREVPVGAVLVLNNRVIGEGWNFAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 91 - protein, 167 aa, synthetic construct
MSEVEFSHEYWMRHALTLAKRARDEREVPVGAVLVLNNRVIGEGWNWAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 92 - protein, 167 aa, synthetic construct
MSEVEFSHEYWMRHALTLAKRARDEREVPVGAVLVLNNRVIGEGWNQAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 93 - protein, 167 aa, synthetic construct
MSEVEFSHEYWMRHALTLAKRARDEREVPVGAVLVLNNRVIGEGWNMAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 94 - protein, 167 aa, synthetic construct
MSEVEFSHEYWMRHALTLAKRAWDEREVPVGAVLVHNNRVIGEGWNRPIGRHDPTAHAQIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 95 - protein, 167 aa, Escherichia coli
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 96 - protein, 178 aa, Escherichia coli
MRRAFITGVFFLSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 97 - protein, 160 aa, Staphylococcus aureus
MGSHMTNDIYFMTLAIEEAKKAAQLGEVPIGAIITKDDEVIARAHNLRETLQQPTAHAEHIAIERAAKVLGSWRLEGCTLYVTLEPCVMCAGTIVMSRIPRVVYGADDPKGGCSGSLMNLLQQSNFNHRAIVDKGVLKEACSTLLTTFFKNLRANKKSTN

SEQ ID NO: 98 - protein, 183 aa, Salmonella enterica
MPPAFITGVTSLSDVELDHEYWMRHALTLAKRAWDEREVPVGAVLVHNHRVIGEGWNRPIGRHDPTAHAEIMALRQGGLVLQNYRLLDTTLYVTLEPCVMCAGAMVHSRIGRVVFGARDAKTGAAGSLIDVLHHPGMNHRVEIIEGVLRDECATLLSDFFRMRRQEIKALKKADRAEGAGPAV

SEQ ID NO: 99 - protein, 164 aa, Shewanella putrefaciens
MDEYWMQVAMQMAEKAEAAGEVPVGAVLVKDGQQIATGYNLSISQHDPTAHAEILCLRSAGKKLENYRLLDATLYITLEPCAMCAGAMVHSRIARVVYGARDEKTGAAGTVVNLLQHPAFNHQVEVTSGVLAEACSAQLSRFFKRRRDEKKALKLAQRAQQGIE

SEQ ID NO: 100 - protein, 173 aa, Haemophilus influenzae
MDAAKVRSEFDEKMMRYALELADKAEALGEIPVGAVLVDDARNIIGEGWNLSIVQSDPTAHAEIIALRNGAKNIQNYRLLNSTLYVTLEPCTMCAGAILHSRIKRLVFGASDYKTGAIGSRFHFFDDYKMNHTLEITSGVLAEECSQKLSTFFQKRREEKKIEKALLKSLSDK

SEQ ID NO: 101 - protein, 84 aa, unidentified
MTNLSDIIEKETGKQLVIQESILMLPEEVEEVIGNKPESDILVHTAYDESTDENVMLLTSDAPEYKPWALVIQDSNGENKIKML

SEQ ID NO: 102 - protein, 219 aa, Homo sapiens
KGHLTRLGLEFFDQPAVPLARAFLGQVLVRRLPNGTELRGRIVETQAYLGPEDEAAHSRGGRQTPRNRGMFMKPGTLYVYIIYGMYFCMNISSQGDGACVLLRALEPLEGLETMRQLRSTLRKGTASRVLKDRELCSGPSKLCQALAINKSFDQRDLAQDEAVWLERGPLEPSEPAVVAAARVGVGHAGEWARKPLRFYVRGSPWVSVVDRVAEQDTQA

SEQ ID NO: 103 - protein, 222 aa, synthetic construct
DLASLRAQQIELASSVIREDRLDKDPPDLIAGAAVGFEQGGEVTRAAMVLLKYPSLELVEYKVARIATTMPYIPGFLSFREYPALLAAWEMLSQKPDLVFVDGHGISHPRRLGVASHFGLLVDVPTIGVAKKRLCGKFEPLSSEPGALAPLMDKGEQLAWVWRSKARCNPLFIATGHRVSVDSALAWVQRCMKGYRLPEPTRWADAVASERPAFVRYTANQP

SEQ ID NO: 104 - protein, 181 aa, Erwinia tasmaniensis
MASRGVNKVILVGNLGQDPEVRYMPNGGAVANITLATSESWRDKQTGETKEKTEWHRVVLFGKLAEVAGEYLRKGSQVYIEGALQTRKWTDQAGVEKYTTEVVVNVGGTMQMLGGRSQGGGASAGGQNGGSNNGWGQPQQPQGGNQFSGGAQQQARPQQQPQQNNAPANNEPPIDFDDDIP

SEQ ID NO: 105 - protein, 209 aa, synthetic construct
MAGAQDFVPHTADLAELAAAAGECRGCGLYRDATQAVFGAGGRSARIMMIGEQPGDKEDLAGLPFVGPAGRLLDRALEAADIDRDALYVTNAVKHFKFTRAAGGKRRIHKTPSRTEVVACRPWLIAEMTSVEPDVVVLLGATAAKALLGNDFRVTQHRGEVLHVDDVPGDPALVATVHPSSLLRGPKEERESAFAGLVDDLRVAADVRP

SEQ ID NO: 106 - protein, 313 aa, synthetic construct
MIGQKTLYSFFSPSPARKRHAPSPEPAVQGTGVAGVPEESGDAAAIPAKKAPAGQEEPGTPPSSPLSAEQLDRIQRNKAAALLRLAARNVPVGFGESWKKHLSGEFGKPYFIKLMGFVAEERKHYTVYPPPHQVFTWTQMCDIKDVKVVILGQEPYHGPNQAHGLCFSVQRPVPPPPSLENIYKELSTDIEDFVHPGHGDLSGWAKQGVLLLNAVLTVRAHQANSHKERGWEQFTDAVVSWLNQNSNGLVFLLWGSYAQKKGSAIDRKRHHVLQTAHPSPLSVYRGFFGCRHFSKTNELLQKSGKKPIDWKEL

SEQ ID NO: 107 - protein, 7 aa, synthetic construct
PKKKRKV

SEQ ID NO: 108 - protein, 16 aa, synthetic construct
KRPAATKKAGQAKKKK

SEQ ID NO: 109 - protein, 9 aa, synthetic construct
PAAKRVKLD

SEQ ID NO: 110 - protein, 11 aa, synthetic construct
RQRRNELKRSP

SEQ ID NO: 111 - protein, 38 aa, synthetic construct; identical to SEQ ID NO: 33

SEQ ID NO: 112 - protein, 42 aa, synthetic construct; identical to SEQ ID NO: 34

SEQ ID NO: 113 - protein, 8 aa, synthetic construct
VSRKRPRP

SEQ ID NO: 114 - protein, 8 aa, synthetic construct
PPKKARED

SEQ ID NO: 115 - protein, 8 aa, synthetic construct
PQPKKKPL

SEQ ID NO: 116 - protein, 12 aa, synthetic construct
SALIKKKKKMAP

SEQ ID NO: 117 - protein, 5 aa, synthetic construct
DRLRR

SEQ ID NO: 118 - protein, 7 aa, synthetic construct
PKQKKRK

SEQ ID NO: 119 - protein, 10 aa, synthetic construct
RKLKKKIKKL

SEQ ID NO: 120 - protein, 10 aa, synthetic construct
REKKKFLKRR

SEQ ID NO: 121 - protein, 20 aa, synthetic construct
KRKGDEVDGVDEVAKKKSKK

SEQ ID NO: 122 - protein, 17 aa, synthetic construct
RKCLQAGMNLEARKTKK

SEQ ID NO: 123 - DNA, 25 bp, synthetic construct
  source 1..25: mol_type = other DNA
tcaaccccacacgcacacactcact

SEQ ID NO: 124 - DNA, 24 bp, synthetic construct
  source 1..24: mol_type = other DNA
caaccccacacgcacacactcact

SEQ ID NO: 125 - DNA, 23 bp, synthetic construct
  source 1..23: mol_type = other DNA
aaccccacacgcacacactcact

SEQ ID NO: 126 - DNA, 22 bp, synthetic construct
  source 1..22: mol_type = other DNA
accccacacgcacacactcact

SEQ ID NO: 127 - DNA, 21 bp, synthetic construct
  source 1..21: mol_type = other DNA
ccccacacgcacacactcact

SEQ ID NO: 128 - DNA, 20 bp, synthetic construct
  source 1..20: mol_type = other DNA
cccacacgcacacactcact

SEQ ID NO: 129 - DNA, 19 bp, synthetic construct
  source 1..19: mol_type = other DNA
ccacacgcacacactcact

SEQ ID NO: 130 - DNA, 18 bp, synthetic construct
  source 1..18: mol_type = other DNA
cacacgcacacactcact

SEQ ID NO: 131 - DNA, 25 bp, synthetic construct
  source 1..25: mol_type = other DNA
actacagacaaggcacttggttgat

SEQ ID NO: 132 - DNA, 24 bp, synthetic construct
  source 1..24: mol_type = other DNA
ctacagacaaggcacttggttgat

SEQ ID NO: 133 - DNA, 23 bp, synthetic construct
  source 1..23: mol_type = other DNA
tacagacaaggcacttggttgat

SEQ ID NO: 134 - DNA, 22 bp, synthetic construct
  source 1..22: mol_type = other DNA
acagacaaggcacttggttgat

SEQ ID NO: 135 - DNA, 21 bp, synthetic construct
  source 1..21: mol_type = other DNA
cagacaaggcacttggttgat

SEQ ID NO: 136 - DNA, 20 bp, synthetic construct
  source 1..20: mol_type = other DNA
agacaaggcacttggttgat

SEQ ID NO: 137 - DNA, 19 bp, synthetic construct
  source 1..19: mol_type = other DNA
gacaaggcacttggttgat

SEQ ID NO: 138 - DNA, 18 bp, synthetic construct
  source 1..18: mol_type = other DNA
acaaggcacttggttgat

SEQ ID NO: 139 - DNA, 26 bp, synthetic construct
  source 1..26: mol_type = other DNA
gtcaaccccacacgcacacactcact

SEQ ID NO: 140 - DNA, 25 bp, synthetic construct
  source 1..25: mol_type = other DNA
gcaaccccacacgcacacactcact

SEQ ID NO: 141 - DNA, 24 bp, synthetic construct
  source 1..24: mol_type = other DNA
gaaccccacacgcacacactcact

SEQ ID NO: 142 - DNA, 23 bp, synthetic construct
  source 1..23: mol_type = other DNA
gaccccacacgcacacactcact

SEQ ID NO: 143 - DNA, 22 bp, synthetic construct
  source 1..22: mol_type = other DNA
gccccacacgcacacactcact

SEQ ID NO: 144 - DNA, 21 bp, synthetic construct
  source 1..21: mol_type = other DNA
gcccacacgcacacactcact

SEQ ID NO: 145 - DNA, 20 bp, synthetic construct
  source 1..20: mol_type = other DNA
gccacacgcacacactcact

SEQ ID NO: 146 - DNA, 19 bp, synthetic construct
  source 1..19: mol_type = other DNA
gcacacgcacacactcact

SEQ ID NO: 147 - DNA, 26 bp, synthetic construct
  source 1..26: mol_type = other DNA
gactacagacaaggcacttggttgat

SEQ ID NO: 148 - DNA, 25 bp, synthetic construct
  source 1..25: mol_type = other DNA
gctacagacaaggcacttggttgat

SEQ ID NO: 149 - DNA, 24 bp, synthetic construct
  source 1..24: mol_type = other DNA
gtacagacaaggcacttggttgat

SEQ ID NO: 150 - DNA, 23 bp, synthetic construct
  source 1..23: mol_type = other DNA
gacagacaaggcacttggttgat

SEQ ID NO: 151 - DNA, 22 bp, synthetic construct
  source 1..22: mol_type = other DNA
gcagacaaggcacttggttgat

SEQ ID NO: 152 - DNA, 21 bp, synthetic construct
  source 1..21: mol_type = other DNA
gagacaaggcacttggttgat

SEQ ID NO: 153 - DNA, 19 bp, synthetic construct
  source 1..19: mol_type = other DNA
gacaaggcacttggttgat

SEQ ID NO: 154 - DNA, 19 bp, synthetic construct
  source 1..19: mol_type = other DNA
gacaaggcacttggttgat

SEQ ID NO: 155 - DNA, 25 bp, synthetic construct
  source 1..25: mol_type = other DNA
gtcacctccaatgactagggtgggc

SEQ ID NO: 156 - DNA, 24 bp, synthetic construct
  source 1..24: mol_type = other DNA
tcacctccaatgactagggtgggc

SEQ ID NO: 157 - DNA, 23 bp, synthetic construct
  source 1..23: mol_type = other DNA
cacctccaatgactagggtgggc

SEQ ID NO: 158 - DNA, 22 bp, synthetic construct
  source 1..22: mol_type = other DNA
acctccaatgactagggtgggc

SEQ ID NO: 159 - DNA, 21 bp, synthetic construct
  source 1..21: mol_type = other DNA
cctccaatgactagggtgggc

SEQ ID NO: 160 - DNA, 20 bp, synthetic construct
  source 1..20: mol_type = other DNA
ctccaatgactagggtgggc

SEQ ID NO: 161 - DNA, 19 bp, synthetic construct
  source 1..19: mol_type = other DNA
tccaatgactagggtgggc

SEQ ID NO: 162 - DNA, 18 bp, synthetic construct
  source 1..18: mol_type = other DNA
ccaatgactagggtgggc

SEQ ID NO: 163 - DNA, 25 bp, synthetic construct
  source 1..25: mol_type = other DNA
ttcacttacagtctggcttatatcc

SEQ ID NO: 164 - DNA, 24 bp, synthetic construct
  source 1..24: mol_type = other DNA
tcacttacagtctggcttatatcc

SEQ ID NO: 165 - DNA, 23 bp, synthetic construct
  source 1..23: mol_type = other DNA
cacttacagtctggcttatatcc

SEQ ID NO: 166 - DNA, 22 bp, synthetic construct
  source 1..22: mol_type = other DNA
acttacagtctggcttatatcc

SEQ ID NO: 167 - DNA, 21 bp, synthetic construct
  source 1..21: mol_type = other DNA
cttacagtctggcttatatcc

SEQ ID NO: 168 - DNA, 20 bp, synthetic construct
  source 1..20: mol_type = other DNA
ttacagtctggcttatatcc

SEQ ID NO: 169 - DNA, 19 bp, synthetic construct
  source 1..19: mol_type = other DNA
tacagtctggcttatatcc

SEQ ID NO: 170 - DNA, 18 bp, synthetic construct
  source 1..18: mol_type = other DNA
acagtctggcttatatcc

SEQ ID NO: 171 - DNA, 25 bp, synthetic construct
  source 1..25: mol_type = other DNA
gtcacctccaatgactagggtgggc

SEQ ID NO: 172 - DNA, 25 bp, synthetic construct
  source 1..25: mol_type = other DNA
gtcacctccaatgactagggtgggc

SEQ ID NO: 173 - DNA, 24 bp, synthetic construct
  source 1..24: mol_type = other DNA
gcacctccaatgactagggtgggc

SEQ ID NO: 174 - DNA, 23 bp, synthetic construct
  source 1..23: mol_type = other DNA
gacctccaatgactagggtgggc

SEQ ID NO: 175 - DNA, 22 bp, synthetic construct
  source 1..22: mol_type = other DNA
gcctccaatgactagggtgggc

SEQ ID NO: 176 - DNA, 21 bp, synthetic construct
  source 1..21: mol_type = other DNA
gctccaatgactagggtgggc

SEQ ID NO: 177 - DNA, 20 bp, synthetic construct
  source 1..20: mol_type = other DNA
gtccaatgactagggtgggc

SEQ ID NO: 178 - DNA, 19 bp, synthetic construct
  source 1..19: mol_type = other DNA
gccaatgactagggtgggc

SEQ ID NO: 179 - DNA, 26 bp, synthetic construct
  source 1..26: mol_type = other DNA
gttcacttacagtctggcttatatcc

SEQ ID NO: 180 - DNA, 25 bp, synthetic construct
  source 1..25: mol_type = other DNA
gtcacttacagtctggcttatatcc

SEQ ID NO: 181 - DNA, 24 bp, synthetic construct
  source 1..24: mol_type = other DNA
gcacttacagtctggcttatatcc

SEQ ID NO: 182 - DNA, 23 bp, synthetic construct
  source 1..23: mol_type = other DNA
gacttacagtctggcttatatcc

SEQ ID NO: 183 - DNA, 22 bp, synthetic construct
  source 1..22: mol_type = other DNA
gcttacagtctggcttatatcc

SEQ ID NO: 184 - DNA, 21 bp, synthetic construct
  source 1..21: mol_type = other DNA
gttacagtctggcttatatcc

SEQ ID NO: 185 - DNA, 20 bp, synthetic construct
  source 1..20: mol_type = other DNA
gtacagtctggcttatatcc

SEQ ID NO: 186 - DNA, 19 bp, synthetic construct
  source 1..19: mol_type = other DNA
gacagtctggcttatatcc

SEQ ID NO: 187 - DNA, 25 bp, synthetic construct
  source 1..25: mol_type = other DNA
gccttctccccgctccaacgccctc

SEQ ID NO: 188 - DNA, 24 bp, synthetic construct
  source 1..24: mol_type = other DNA
ccttctccccgctccaacgccctc

SEQ ID NO: 189 - DNA, 23 bp, synthetic construct
  source 1..23: mol_type = other DNA
cttctccccgctccaacgccctc

SEQ ID NO: 190 - DNA, 22 bp, synthetic construct
  source 1..22: mol_type = other DNA
ttctccccgctccaacgccctc

SEQ ID NO: 191 - DNA, 21 bp, synthetic construct
  source 1..21: mol_type = other DNA
tctccccgctccaacgccctc

SEQ ID NO: 192 - DNA, 20 bp, synthetic construct
  source 1..20: mol_type = other DNA
ctccccgctccaacgccctc

SEQ ID NO: 193 - DNA, 19 bp, synthetic construct
  source 1..19: mol_type = other DNA
tccccgctccaacgccctc

SEQ ID NO: 194 - DNA, 18 bp, synthetic construct
  source 1..18: mol_type = other DNA
ccccgctccaacgccctc

SEQ ID NO: 195 - DNA, 25 bp, synthetic construct
  source 1..25: mol_type = other DNA
ttcacttacagtctggcttatatcc

SEQ ID NO: 196 - DNA, 24 bp, synthetic construct
  source 1..24: mol_type = other DNA
tcacttacagtctggcttatatcc

SEQ ID NO: 197 - DNA, 23 bp, synthetic construct
  source 1..23: mol_type = other DNA
cacttacagtctggcttatatcc

SEQ ID NO: 198 - DNA, 22 bp, synthetic construct
  source 1..22: mol_type = other DNA
acttacagtctggcttatatcc

SEQ ID NO: 199 - DNA, 21 bp, synthetic construct
  source 1..21: mol_type = other DNA
cttacagtctggcttatatcc

SEQ ID NO: 200 - DNA, 20 bp, synthetic construct
  source 1..20: mol_type = other DNA
ttacagtctggcttatatcc

SEQ ID NO: 201 - DNA, 19 bp, synthetic construct
  source 1..19: mol_type = other DNA
tacagtctggcttatatcc

SEQ ID NO: 202 - DNA, 18 bp, synthetic construct
  source 1..18: mol_type = other DNA
acagtctggcttatatcc

SEQ ID NO: 203 - DNA, 25 bp, synthetic construct
  source 1..25: mol_type = other DNA
gccttctccccgctccaacgccctc

SEQ ID NO: 204 - DNA, 25 bp, synthetic construct
  source 1..25: mol_type = other DNA
gccttctccccgctccaacgccctc

SEQ ID NO: 205 - DNA, 24 bp, synthetic construct
  source 1..24: mol_type = other DNA
gcttctccccgctccaacgccctc

SEQ ID NO: 206 - DNA, 23 bp, synthetic construct
  source 1..23: mol_type = other DNA
gttctccccgctccaacgccctc

SEQ ID NO: 207 - DNA, 22 bp, synthetic construct
  source 1..22: mol_type = other DNA
gtctccccgctccaacgccctc

SEQ ID NO: 208 - DNA, 21 bp, synthetic construct
  source 1..21: mol_type = other DNA
gctccccgctccaacgccctc

SEQ ID NO: 209 - DNA, 20 bp, synthetic construct
  source 1..20: mol_type = other DNA
gtccccgctccaacgccctc

SEQ ID NO: 210 - DNA, 19 bp, synthetic construct
  source 1..19: mol_type = other DNA
gccccgctccaacgccctc

SEQ ID NO: 211 - DNA, 26 bp, synthetic construct
  source 1..26: mol_type = other DNA
gttcacttacagtctggcttatatcc

SEQ ID NO: 212 - DNA, 25 bp, synthetic construct
  source 1..25: mol_type = other DNA
gtcacttacagtctggcttatatcc

SEQ ID NO: 213 - DNA, 24 bp, synthetic construct
  source 1..24: mol_type = other DNA
gcacttacagtctggcttatatcc

SEQ ID NO: 214 - DNA, 23 bp, synthetic construct
  source 1..23: mol_type = other DNA
gacttacagtctggcttatatcc

SEQ ID NO: 215 - DNA, 22 bp, synthetic construct
  source 1..22: mol_type = other DNA
gcttacagtctggcttatatcc

SEQ ID NO: 216 - DNA, 21 bp, synthetic construct
  source 1..21: mol_type = other DNA
gttacagtctggcttatatcc

SEQ ID NO: 217 - DNA, 20 bp, synthetic construct
  source 1..20: mol_type = other DNA
gtacagtctggcttatatcc

SEQ ID NO: 218 - DNA, 19 bp, synthetic construct
  source 1..19: mol_type = other DNA
gacagtctggcttatatcc

SEQ ID NO: 219 - DNA, 25 bp, synthetic construct
  source 1..25: mol_type = other DNA
ggcctgagtccgagcagaagaagaa

SEQ ID NO: 220 - DNA, 24 bp, synthetic construct
  source 1..24: mol_type = other DNA
gcctgagtccgagcagaagaagaa

SEQ ID NO: 221 - DNA, 23 bp, synthetic construct
  source 1..23: mol_type = other DNA
cctgagtccgagcagaagaagaa

SEQ ID NO: 222 - DNA, 22 bp, synthetic construct
  source 1..22: mol_type = other DNA
ctgagtccgagcagaagaagaa

SEQ ID NO: 223 - DNA, 21 bp, synthetic construct
  source 1..21: mol_type = other DNA
tgagtccgagcagaagaagaa

SEQ ID NO: 224 - DNA, 20 bp, synthetic construct
  source 1..20: mol_type = other DNA
gagtccgagcagaagaagaa

SEQ ID NO: 225 - DNA, 19 bp, synthetic construct
  source 1..19: mol_type = other DNA
agtccgagcagaagaagaa

SEQ ID NO: 226 - DNA, 18 bp, synthetic construct
  source 1..18: mol_type = other DNA
gtccgagcagaagaagaa

SEQ ID NO: 227 - DNA, 25 bp, synthetic construct
  source 1..25: mol_type = other DNA
agggtgggcaaccacaaacccacga

SEQ ID NO: 228 - DNA, 24 bp, synthetic construct
  source 1..24: mol_type = other DNA
gggtgggcaaccacaaacccacga

SEQ ID NO: 229 - DNA, 23 bp, synthetic construct
  source 1..23: mol_type = other DNA
ggtgggcaaccacaaacccacga

SEQ ID NO: 230 - DNA, 22 bp, synthetic construct
  source 1..22: mol_type = other DNA
gtgggcaaccacaaacccacga

SEQ ID NO: 231 - DNA, 21 bp, synthetic construct
  source 1..21: mol_type = other DNA
tgggcaaccacaaacccacga

SEQ ID NO: 232 - DNA, 20 bp, synthetic construct
  source 1..20: mol_type = other DNA
gggcaaccacaaacccacga

SEQ ID NO: 233 - DNA, 19 bp, synthetic construct
  source 1..19: mol_type = other DNA
ggcaaccacaaacccacga

SEQ ID NO: 234 - DNA, 18 bp, synthetic construct
  source 1..18: mol_type = other DNA
gcaaccacaaacccacga

SEQ ID NO: 235 - DNA, 25 bp, synthetic construct
  source 1..25: mol_type = other DNA
ggcctgagtccgagcagaagaagaa

SEQ ID NO: 236 - DNA, 24 bp, synthetic construct
  source 1..24: mol_type = other DNA
gcctgagtccgagcagaagaagaa

SEQ ID NO: 237 - DNA, 24 bp, synthetic construct
  source 1..24: mol_type = other DNA
gcctgagtccgagcagaagaagaa

SEQ ID NO: 238 - DNA, 23 bp, synthetic construct
  source 1..23: mol_type = other DNA
gctgagtccgagcagaagaagaa

SEQ ID NO: 239 - DNA, 22 bp, synthetic construct
  source 1..22: mol_type = other DNA
gtgagtccgagcagaagaagaa

SEQ ID NO: 240 - DNA, 20 bp, synthetic construct
  source 1..20: mol_type = other DNA
gagtccgagcagaagaagaa

SEQ ID NO: 241 - DNA, 20 bp, synthetic construct
  source 1..20: mol_type = other DNA
gagtccgagcagaagaagaa

SEQ ID NO: 242 - DNA, 18 bp, synthetic construct
  source 1..18: mol_type = other DNA
gtccgagcagaagaagaa

SEQ ID NO: 243 - DNA, 26 bp, synthetic construct
  source 1..26: mol_type = other DNA
gagggtgggcaaccacaaacccacga

SEQ ID NO: 244 - DNA, 24 bp, synthetic construct
  source 1..24: mol_type = other DNA
gggtgggcaaccacaaacccacga

SEQ ID NO: 245 - DNA, 23 bp, synthetic construct
  source 1..23: mol_type = other DNA
ggtgggcaaccacaaacccacga

SEQ ID NO: 246 - DNA, 22 bp, synthetic construct
  source 1..22: mol_type = other DNA
gtgggcaaccacaaacccacga

SEQ ID NO: 247 - DNA, 22 bp, synthetic construct
  source 1..22: mol_type = other DNA
gtgggcaaccacaaacccacga

SEQ ID NO: 248 - DNA, 20 bp, synthetic construct
  source 1..20: mol_type = other DNA
gggcaaccacaaacccacga

SEQ ID NO: 249 - DNA, 19 bp, synthetic construct
  source 1..19: mol_type = other DNA
ggcaaccacaaacccacga

SEQ ID NO: 250 - DNA, 18 bp, synthetic construct
  source 1..18: mol_type = other DNA
gcaaccacaaacccacga

SEQ ID NO: 251 - DNA, 25 bp, synthetic construct
  source 1..25: mol_type = other DNA
ggggttgagggcgttggagcgggga

SEQ ID NO: 252 - DNA, 24 bp, synthetic construct
  source 1..24: mol_type = other DNA
gggttgagggcgttggagcgggga

SEQ ID NO: 253 - DNA, 23 bp, synthetic construct
  source 1..23: mol_type = other DNA
ggttgagggcgttggagcgggga

SEQ ID NO: 254 - DNA, 22 bp, synthetic construct
  source 1..22: mol_type = other DNA
gttgagggcgttggagcgggga

SEQ ID NO: 255 - DNA, 21 bp, synthetic construct
  source 1..21: mol_type = other DNA
ttgagggcgttggagcgggga

SEQ ID NO: 256 - DNA, 20 bp, synthetic construct
  source 1..20: mol_type = other DNA
tgagggcgttggagcgggga

SEQ ID NO: 257 - DNA, 19 bp, synthetic construct
  source 1..19: mol_type = other DNA
gagggcgttggagcgggga

SEQ ID NO: 258 - DNA, 18 bp, synthetic construct
  source 1..18: mol_type = other DNA
agggcgttggagcgggga

SEQ ID NO: 259 - DNA, 25 bp, synthetic construct
  source 1..25: mol_type = other DNA
tccaatgactagggtgggcaaccac

SEQ ID NO: 260 - DNA, 24 bp, synthetic construct
  source 1..24: mol_type = other DNA
ccaatgactagggtgggcaaccac

SEQ ID NO: 261 - DNA, 23 bp, synthetic construct
  source 1..23: mol_type = other DNA
caatgactagggtgggcaaccac

SEQ ID NO: 262 - DNA, 22 bp, synthetic construct
  source 1..22: mol_type = other DNA
aatgactagggtgggcaaccac

SEQ ID NO: 263 - DNA, 21 bp, synthetic construct
  source 1..21: mol_type = other DNA
atgactagggtgggcaaccac

SEQ ID NO: 264 - DNA, 20 bp, synthetic construct
  source 1..20: mol_type = other DNA
tgactagggtgggcaaccac

SEQ ID NO: 265 - DNA, 19 bp, synthetic construct
  source 1..19: mol_type = other DNA
gactagggtgggcaaccac

SEQ ID NO: 266 - DNA, 18 bp, synthetic construct
  source 1..18: mol_type = other DNA
actagggtgggcaaccac

SEQ ID NO: 267 - DNA, 25 bp, synthetic construct
  source 1..25: mol_type = other DNA
ggggttgagggcgttggagcgggga

SEQ ID NO: 268 - DNA, 24 bp, synthetic construct
  source 1..24: mol_type = other DNA
gggttgagggcgttggagcgggga

SEQ ID NO: 269 - DNA, 23 bp, synthetic construct
  source 1..23: mol_type = other DNA
ggttgagggcgttggagcgggga

SEQ ID NO: 270 - DNA, 22 bp, synthetic construct
  source 1..22: mol_type = other DNA
gttgagggcgttggagcgggga

SEQ ID NO: 271 - DNA, 22 bp, synthetic construct
  source 1..22: mol_type = other DNA
gttgagggcgttggagcgggga

SEQ ID NO: 272 - DNA, 21 bp, synthetic construct
  source 1..21: mol_type = other DNA
gtgagggcgttggagcgggga

SEQ ID NO: 273 - DNA, 19 bp, synthetic construct
  source 1..19: mol_type = other DNA
gagggcgttggagcgggga

SEQ ID NO: 274 - DNA, 19 bp, synthetic construct
  source 1..19: mol_type = other DNA
gagggcgttggagcgggga

SEQ ID NO: 275 - DNA, 26 bp, synthetic construct
  source 1..26: mol_type = other DNA
gtccaatgactagggtgggcaaccac

SEQ ID NO: 276 - DNA, 25 bp, synthetic construct
  source 1..25: mol_type = other DNA
gccaatgactagggtgggcaaccac

SEQ ID NO: 277 - DNA, 24 bp, synthetic construct
  source 1..24: mol_type = other DNA
gcaatgactagggtgggcaaccac

SEQ ID NO: 278 - DNA, 23 bp, synthetic construct
  source 1..23: mol_type = other DNA
gaatgactagggtgggcaaccac

SEQ ID NO: 279 - DNA, 22 bp, synthetic construct
  source 1..22: mol_type = other DNA
gatgactagggtgggcaaccac

SEQ ID NO: 280 - DNA, 21 bp, synthetic construct
  source 1..21: mol_type = other DNA
gtgactagggtgggcaaccac

SEQ ID NO: 281 - DNA, 19 bp, synthetic construct
  source 1..19: mol_type = other DNA
gactagggtgggcaaccac

SEQ ID NO: 282 - DNA, 19 bp, synthetic construct
  source 1..19: mol_type = other DNA
gactagggtgggcaaccac

SEQ ID NO: 283 - DNA, 25 bp, synthetic construct
  source 1..25: mol_type = other DNA
gtcacctccaatgactagggtgggc

SEQ ID NO: 284 - DNA, 24 bp, synthetic construct
  source 1..24: mol_type = other DNA
tcacctccaatgactagggtgggc

SEQ ID NO: 285 - DNA, 23 bp, synthetic construct
  source 1..23: mol_type = other DNA
cacctccaatgactagggtgggc

SEQ ID NO: 286 - DNA, 22 bp, synthetic construct
  source 1..22: mol_type = other DNA
acctccaatgactagggtgggc

SEQ ID NO: 287 - DNA, 21 bp, synthetic construct
  source 1..21: mol_type = other DNA
cctccaatgactagggtgggc

SEQ ID NO: 288 - DNA, 20 bp, synthetic construct
  source 1..20: mol_type = other DNA
ctccaatgactagggtgggc

SEQ ID NO: 289 - DNA, 19 bp, synthetic construct
  source 1..19: mol_type = other DNA
tccaatgactagggtgggc

SEQ ID NO: 290 - DNA, 18 bp, synthetic construct
  source 1..18: mol_type = other DNA
ccaatgactagggtgggc

SEQ ID NO: 291 - DNA, 25 bp, synthetic construct
  source 1..25: mol_type = other DNA
aaccccacacgcacacactcactca

SEQ ID NO: 292 - DNA, 24 bp, synthetic construct
  source 1..24: mol_type = other DNA
accccacacgcacacactcactca

SEQ ID NO: 293 - DNA, 23 bp, synthetic construct
  source 1..23: mol_type = other DNA
ccccacacgcacacactcactca

SEQ ID NO: 294 - DNA, 22 bp, synthetic construct
  source 1..22: mol_type = other DNA
cccacacgcacacactcactca

SEQ ID NO: 295 - DNA, 21 bp, synthetic construct
  source 1..21: mol_type = other DNA
ccacacgcacacactcactca

SEQ ID NO: 296 - DNA, 20 bp, synthetic construct
  source 1..20: mol_type = other DNA
cacacgcacacactcactca

SEQ ID NO: 297 - DNA, 19 bp, synthetic construct
  source 1..19: mol_type = other DNA
acacgcacacactcactca

SEQ ID NO: 298 - DNA, 18 bp, synthetic construct
  source 1..18: mol_type = other DNA
cacgcacacactcactca

SEQ ID NO: 299 - DNA, 25 bp, synthetic construct
  source 1..25: mol_type = other DNA
gtcacctccaatgactagggtgggc

SEQ ID NO: 300 - DNA, 25 bp, synthetic construct
  source 1..25: mol_type = other DNA
gtcacctccaatgactagggtgggc

SEQ ID NO: 301 - DNA, 24 bp, synthetic construct
  source 1..24: mol_type = other DNA
gcacctccaatgactagggtgggc

SEQ ID NO: 302 - DNA, 23 bp, synthetic construct
  source 1..23: mol_type = other DNA
gacctccaatgactagggtgggc

SEQ ID NO: 303 - DNA, 22 bp, synthetic construct
  source 1..22: mol_type = other DNA
gcctccaatgactagggtgggc

SEQ ID NO: 304 - DNA, 21 bp, synthetic construct
  source 1..21: mol_type = other DNA
gctccaatgactagggtgggc

SEQ ID NO: 305 - DNA, 20 bp, synthetic construct
  source 1..20: mol_type = other DNA
gtccaatgactagggtgggc

SEQ ID NO: 306 - DNA, 19 bp, synthetic construct
  source 1..19: mol_type = other DNA
gccaatgactagggtgggc

SEQ ID NO: 307 - DNA, 26 bp, synthetic construct
  source 1..26: mol_type = other DNA
gaaccccacacgcacacactcactca

SEQ ID NO: 308 - DNA, 25 bp, synthetic construct
  source 1..25: mol_type = other DNA
gaccccacacgcacacactcactca

SEQ ID NO: 309 - DNA, 24 bp, synthetic construct
  source 1..24: mol_type = other DNA
gccccacacgcacacactcactca

SEQ ID NO: 310 - DNA, 23 bp, synthetic construct
  source 1..23: mol_type = other DNA
gcccacacgcacacactcactca

SEQ ID NO: 311 - DNA, 22 bp, synthetic construct
  source 1..22: mol_type = other DNA
gccacacgcacacactcactca

SEQ ID NO: 312 - DNA, 21 bp, synthetic construct
  source 1..21: mol_type = other DNA
gcacacgcacacactcactca

SEQ ID NO: 313 - DNA, 20 bp, synthetic construct
  source 1..20: mol_type = other DNA
gacacgcacacactcactca

SEQ ID NO: 314 - DNA, 19 bp, synthetic construct
  source 1..19: mol_type = other DNA
gcacgcacacactcactca

SEQ ID NO: 315 - RNA, 156 nt, synthetic construct
  source 1..156: mol_type = other RNA
gtttcaatcaaccgatgaaaatcggtccggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 316 - RNA, 154 nt, synthetic construct
  source 1..154: mol_type = other RNA
gtttcaatcaaccgagaaatcggtccggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 317 - RNA, 152 nt, synthetic construct
  source 1..152: mol_type = other RNA
gtttcaatcaaccggaaacggtccggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 318 - RNA, 150 nt, synthetic construct
  source 1..150: mol_type = other RNA
gtttcaatcaaccgaaaggtccggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 319 - RNA, 148 nt, synthetic construct
  source 1..148: mol_type = other RNA
gtttcaatcaacgaaagtccggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 320 - RNA, 146 nt, synthetic construct
  source 1..146: mol_type = other RNA
gtttcaatcaagaaatccggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 321 - RNA, 142 nt, synthetic construct
  source 1..142: mol_type = other RNA
gtttcaatcaatccggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 322 - RNA, 141 nt, synthetic construct
  source 1..141: mol_type = other RNA
gtttcaatcgaaaggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 323 - RNA, 139 nt, synthetic construct
  source 1..139: mol_type = other RNA
gtttcaatgaaagttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 324 - RNA, 137 nt, synthetic construct
  source 1..137: mol_type = other RNA
gtttcaagaaattgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 325 - RNA, 135 nt, synthetic construct
  source 1..135: mol_type = other RNA
gtttcagaaatgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 326 - RNA, 154 nt, synthetic construct
  source 1..154: mol_type = other RNA
gtttcaaaaccgatagaaatatcggtccttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 327 - RNA, 153 nt, synthetic construct
  source 1..153: mol_type = other RNA
gtttcaaaccgatagaaatatcggtccttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 328 - RNA, 155 nt, synthetic construct
  source 1..155: mol_type = other RNA
gtttcaaaccgatagaaatatcggtccggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 329 - RNA, 156 nt, synthetic construct
  source 1..156: mol_type = other RNA
gtttcaaaccgatagaaatatcggtcctgattgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 330 - RNA, 152 nt, synthetic construct
  source 1..152: mol_type = other RNA
gtttcaaaaccgatgaaaatcggtccttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 331 - RNA, 151 nt, synthetic construct
  source 1..151: mol_type = other RNA
gtttcaaaccgatgaaaatcggtccttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 332 - RNA, 153 nt, synthetic construct
  source 1..153: mol_type = other RNA
gtttcaaaccgatgaaaatcggtccggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 333 - RNA, 154 nt, synthetic construct
  source 1..154: mol_type = other RNA
gtttcaaaccgatgaaaatcggtcctgattgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 334 - RNA, 157 nt, synthetic construct
  source 1..157: mol_type = other RNA
gtttcaatcaaccgatagaaatatcggttggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 335 - RNA, 158 nt, synthetic construct
  source 1..158: mol_type = other RNA
gtttcaatcgaccgatagaaatatcggtccggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 336 - RNA, 158 nt, synthetic construct
  source 1..158: mol_type = other RNA
gtttcaatcaaccgatagaaatatcggttcggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 337 - RNA, 158 nt, synthetic construct
  source 1..158: mol_type = other RNA
gtttcaatcaaccgatagaaatatcggtctggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 338 - RNA, 157 nt, synthetic construct
  source 1..157: mol_type = other RNA
gtttcaatcgaccgatagaaatatcggtcggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 339 - RNA, 157 nt, synthetic construct
  source 1..157: mol_type = other RNA
tttcaatcaaccgatagaaatatcggtccggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 340 - RNA, 156 nt, synthetic construct
  source 1..156: mol_type = other RNA
gtttcaatcaaccgatagaaatatcggtccggttgaaatgcatcggtctgaaggatgcactccgggatagggcagtcccggctcttgctgtttccccggtaagacctcggaagcaagtccttcagcaagtcgaaagacacgatgtgagcctatttt

SEQ ID NO: 341 - RNA, 157 nt, synthetic construct
  source 1..157: mol_type = other RNA
gtttcaatcaaccgatagaaatatcggtccggttgaaaagagcatcggtctgaaggatgcactccgggatagggcagtcccggctcttgctgtttccccggtaagacctcggaagcaagtccttcagcaagtcgaaagacacgatgtgagcctattt

SEQ ID NO: 342 - RNA, 156 nt, synthetic construct
  source 1..156: mol_type = other RNA
gtttcaatcaaccgatagaaatatcggtccggttgaaaagagcatcggtctgaaggatgcactccgggatagggcagtcccggctcttgctgtttccccggtaagacctcggaagcaagtccttcagcaagtcgaaagacacgatgtgagcctatt

SEQ ID NO: 343 - RNA, 155 nt, synthetic construct
  source 1..155: mol_type = other RNA
gtttcaatcaaccgatagaaatatcggtccggttgaaaagagcatcggtctgaaggatgcactccgggatagggcagtcccggctcttgctgtttccccggtaagacctcggaagcaagtccttcagcaagtcgaaagacacgatgtgagcctat

SEQ ID NO: 344 - RNA, 154 nt, synthetic construct
  source 1..154: mol_type = other RNA
gtttcaatcaaccgatagaaatatcggtccggttgaaaagagcatcggtctgaaggatgcactccgggatagggcagtcccggctcttgctgtttccccggtaagacctcggaagcaagtccttcagcaagtcgaaagacacgatgtgagccta

SEQ ID NO: 345 - RNA, 153 nt, synthetic construct
  source 1..153: mol_type = other RNA
gtttcaatcaaccgatagaaatatcggtccggttgaaaagagcatcggtctgaaggatgcactccgggatagggcagtcccggctcttgctgtttccccggtaagacctcggaagcaagtccttcagcaagtcgaaagacacgatgtgagcct

SEQ ID NO: 346 - RNA, 152 nt, synthetic construct
  source 1..152: mol_type = other RNA
gtttcaatcaaccgatagaaatatcggtccggttgaaaagagcatcggtctgaaggatgcactccgggatagggcagtcccggctcttgctgtttccccggtaagacctcggaagcaagtccttcagcaagtcgaaagacacgatgtgagcc

SEQ ID NO: 347 - RNA, 151 nt, synthetic construct
  source 1..151: mol_type = other RNA
gtttcaatcaaccgatagaaatatcggtccggttgaaaagagcatcggtctgaaggatgcactccgggatagggcagtcccggctcttgctgtttccccggtaagacctcggaagcaagtccttcagcaagtcgaaagacacgatgtgagc

SEQ ID NO: 348 - RNA, 150 nt, synthetic construct
  source 1..150: mol_type = other RNA
gtttcaatcaaccgatagaaatatcggtccggttgaaaagagcatcggtctgaaggatgcactccgggatagggcagtcccggctcttgctgtttccccggtaagacctcggaagcaagtccttcagcaagtcgaaagacacgatgtgag

SEQ ID NO: 349 - RNA, 149 nt, synthetic construct
  source 1..149: mol_type = other RNA
gtttcaatcaaccgatagaaatatcggtccggttgaaaagagcatcggtctgaaggatgcactccgggatagggcagtcccggctcttgctgtttccccggtaagacctcggaagcaagtccttcagcaagtcgaaagacacgatgtga

SEQ ID NO: 350 - RNA, 148 nt, synthetic construct
  source 1..148: mol_type = other RNA
gtttcaatcaaccgatagaaatatcggtccggttgaaaagagcatcggtctgaaggatgcactccgggatagggcagtcccggctcttgctgtttccccggtaagacctcggaagcaagtccttcagcaagtcgaaagacacgatgtg

SEQ ID NO: 351 - RNA, 147 nt, synthetic construct
  source 1..147: mol_type = other RNA
gtttcaatcaaccgatagaaatatcggtccggttgaaaagagcatcggtctgaaggatgcactccgggatagggcagtcccggctcttgctgtttccccggtaagacctcggaagcaagtccttcagcaagtcgaaagacacgatgt

SEQ ID NO: 352 - RNA, 157 nt, synthetic construct
  source 1..157: mol_type = other RNA
gtttcaatcaaccgatagaaatatcggtccggttgaaaagagcatcggctgaaggatgcactccgggatagggcagtcccggctcttgctgtttccccggtaagacctcggaagcaagtccttcagcaagtcgaaagacacgatgtgagcctatttt

SEQ ID NO: 353 - RNA, 156 nt, synthetic construct
  source 1..156: mol_type = other RNA
gtttcaatcaaccgatagaaatatcggtccggttgaaaagagcatcggtctgaaggatgcactccgggatagggcagtcccggctcttgctgtttccccggtaagacctcggaagcaagtccttcagcaagtgaaaacacgatgtgagcctatttt

SEQ ID NO: 354 - RNA, 154 nt, synthetic construct
  source 1..154: mol_type = other RNA
gtttcaatcaaccgatagaaatatcggtccggttgaaaagagcatcggtctgaaggatgcactccgggatagggcagtcccggctcttgctgtttccccggtaagacctcggaagcaagtccttcagcaaggaaacacgatgtgagcctatttt

SEQ ID NO: 355 - RNA, 152 nt, synthetic construct
  source 1..152: mol_type = other RNA
gtttcaatcaaccgatagaaatatcggtccggttgaaaagagcatcggtctgaaggatgcactccgggatagggcagtcccggctcttgctgtttccccggtaagacctcggaagcaagtccttcagcaagaaaacgatgtgagcctatttt

SEQ ID NO: 356 - RNA, 151 nt, synthetic construct
  source 1..151: mol_type = other RNA
gtttcaatcaaccgatagaaatatcggtccggttgaaaagagcatcggtctgaaggatgcactccgggatagggcagtcccggctcttgctgtttccccggtaagacctcggaagcaagtccttcagcagaaaacgatgtgagcctatttt

SEQ ID NO: 357 - RNA, 145 nt, synthetic construct
  source 1..145: mol_type = other RNA
gtttcaatcaaccgatagaaatatcggtccggttgaaaagagcatcggctgaaggatgcactccgggatagggcagtcccggctcttgctgtttccccggtaagacctcggaagcaagtccttcagcacgatgtgagcctatttt

SEQ ID NO: 358 - RNA, 147 nt, synthetic construct
  source 1..147: mol_type = other RNA
gtttcaatcaaccgatagaaatatcggtccggttgaaaagagcatcggctgaaggatgcactccgggatagggcagtcccggctcttgctgtttccccggtaagacctcggaagcaagtccttcagcttacgatgtgagcctatttt

SEQ ID NO: 359 - RNA, 148 nt, synthetic construct
  source 1..148: mol_type = other RNA
gtttcaatcaaccgatagaaatatcggtccggttgaaaagagcatcggctgaaggatgcactccgggatagggcagtcccggctcttgctgtttccccggtaagacctcggaagcaagtccttcagcgagccgatgtgagcctatttt

SEQ ID NO: 360 - RNA, 149 nt, synthetic construct
  source 1..149: mol_type = other RNA
gtttcaatcaaccgatagaaatatcggtccggttgaaaagagcatcgagctgaaggatgcactccgggatagggcagtcccggctcttgctgtttccccggtaagacctcggaagcaagtccttcagcgagccgatgtgagcctatttt

SEQ ID NO: 361 - RNA, 145 nt, synthetic construct
  source 1..145: mol_type = other RNA
gtttcaatcaaccgatagaaatatcggtccggttgaaaagagcatcggtctgaaggatgcactccgggatagggcagtcccggctcttgctgtttccccggtaagacctcggaagcaagtccttcagccgatgtgagcctatttt

SEQ ID NO: 362 - RNA, 144 nt, synthetic construct
  source 1..144: mol_type = other RNA
gtttcaatcaaccgatagaaatatcggtccggttgaaaagagcatcggctgaaggatgcactccgggatagggcagtcccggctcttgctgtttccccggtaagacctcggaagcaagtccttcagccgatgtgagcctatttt

SEQ ID NO: 363 - RNA, 156 nt, synthetic construct
  source 1..156: mol_type = other RNA
gtttcaatcaaccgatagaaatatcggtccggttgaaaagagcatcggtctgaaggatgcactccgggaagggcatcccggctcttgctgtttccccggtaagacctcggaagcaagtccttcagcaagtcgaaagacacgatgtgagcctatttt

SEQ ID NO: 364 - RNA, 154 nt, synthetic construct
  source 1..154: mol_type = other RNA
gtttcaatcaaccgatagaaatatcggtccggttgaaaagagcatcggtctgaaggatgcactccgggagggcacccggctcttgctgtttccccggtaagacctcggaagcaagtccttcagcaagtcgaaagacacgatgtgagcctatttt

SEQ ID NO: 365 - RNA, 152 nt, synthetic construct
  source 1..152: mol_type = other RNA
gtttcaatcaaccgatagaaatatcggtccggttgaaaagagcatcggtctgaaggatgcactccggagggcaccggctcttgctgtttccccggtaagacctcggaagcaagtccttcagcaagtcgaaagacacgatgtgagcctatttt

SEQ ID NO: 366 - RNA, 150 nt, synthetic construct
  source 1..150: mol_type = other RNA
gtttcaatcaaccgatagaaatatcggtccggttgaaaagagcatcggtctgaaggatgcactccgagggcacggctcttgctgtttccccggtaagacctcggaagcaagtccttcagcaagtcgaaagacacgatgtgagcctatttt

SEQ ID NO: 367 - RNA, 148 nt, synthetic construct
  source 1..148: mol_type = other RNA
gtttcaatcaaccgatagaaatatcggtccggttgaaaagagcatcggtctgaaggatgcactccagggcaggctcttgctgtttccccggtaagacctcggaagcaagtccttcagcaagtcgaaagacacgatgtgagcctatttt

SEQ ID NO: 368 - RNA, 146 nt, synthetic construct
  source 1..146: mol_type = other RNA
gtttcaatcaaccgatagaaatatcggtccggttgaaaagagcatcggtctgaaggatgcactcagggcagctcttgctgtttccccggtaagacctcggaagcaagtccttcagcaagtcgaaagacacgatgtgagcctatttt

SEQ ID NO: 369 - RNA, 144 nt, synthetic construct
  source 1..144: mol_type = other RNA
gtttcaatcaaccgatagaaatatcggtccggttgaaaagagcatcggtctgaaggatgcactagggcactcttgctgtttccccggtaagacctcggaagcaagtccttcagcaagtcgaaagacacgatgtgagcctatttt

SEQ ID NO: 370 - RNA, 138 nt, synthetic construct
  source 1..138: mol_type = other RNA
gtttcaatcaaccgatagaaatatcggtccggttgaaaagagcatcggtctgaaggatgcaagggcatgctgtttccccggtaagacctcggaagcaagtccttcagcaagtcgaaagacacgatgtgagcctatttt

SEQ ID NO: 371 - RNA, 136 nt, synthetic construct
  source 1..136: mol_type = other RNA
gtttcaatcaaccgatagaaatatcggtccggttgaaaagagcatcggtctgaaggatgcagggcagctgtttccccggtaagacctcggaagcaagtccttcagcaagtcgaaagacacgatgtgagcctatttt

SEQ ID NO: 372 - RNA, 134 nt, synthetic construct
  source 1..134: mol_type = other RNA
gtttcaatcaaccgatagaaatatcggtccggttgaaaagagcatcggtctgaaggatgagggcactgtttccccggtaagacctcggaagcaagtccttcagcaagtcgaaagacacgatgtgagcctatttt

SEQ ID NO: 373 - RNA, 128 nt, synthetic construct
  source 1..128: mol_type = other RNA
gtttcaatcaaccgatagaaatatcggtccggttgaaaagagcatcggtctgaaggatgctgtttccccggtaagacctcggaagcaagtccttcagcaagtcgaaagacacgatgtgagcctatttt

SEQ ID NO: 374 - RNA, 156 nt, synthetic construct
  source 1..156: mol_type = other RNA
gtttcaatcaaccgatagaaatatcggtccggttgaaaagagcatcggtctgaaggatgcactccgggatagggcagtcccggctcttgctgtttccccggaagcctcggaagcaagtccttcagcaagtcgaaagacacgatgtgagcctatttt

SEQ ID NO: 375 - RNA, 154 nt, synthetic construct
  source 1..154: mol_type = other RNA
gtttcaatcaaccgatagaaatatcggtccggttgaaaagagcatcggtctgaaggatgcactccgggatagggcagtcccggctcttgctgtttccccgaagctcggaagcaagtccttcagcaagtcgaaagacacgatgtgagcctatttt

SEQ ID NO: 376 - RNA, 152 nt, synthetic construct
  source 1..152: mol_type = other RNA
gtttcaatcaaccgatagaaatatcggtccggttgaaaagagcatcggtctgaaggatgcactccgggatagggcagtcccggctcttgctgtttccccaagtcggaagcaagtccttcagcaagtcgaaagacacgatgtgagcctatttt

SEQ ID NO: 377 - RNA, 148 nt, synthetic construct
  source 1..148: mol_type = other RNA
gtttcaatcaaccgatagaaatatcggtccggttgaaaagagcatcggtctgaaggatgcactccgggatagggcagtcccggctcttgctgtttccaagggaagcaagtccttcagcaagtcgaaagacacgatgtgagcctatttt

SEQ ID NO: 378 - RNA, 146 nt, synthetic construct
  source 1..146: mol_type = other RNA
gtttcaatcaaccgatagaaatatcggtccggttgaaaagagcatcggtctgaaggatgcactccgggatagggcagtcccggctcttgctgtttcaaggaagcaagtccttcagcaagtcgaaagacacgatgtgagcctatttt

SEQ ID NO: 379 - RNA, 144 nt, synthetic construct
  source 1..144: mol_type = other RNA
gtttcaatcaaccgatagaaatatcggtccggttgaaaagagcatcggtctgaaggatgcactccgggatagggcagtcccggctcttgctgtttaagaagcaagtccttcagcaagtcgaaagacacgatgtgagcctatttt

SEQ ID NO: 380 - RNA, 142 nt, synthetic construct
  source 1..142: mol_type = other RNA
gtttcaatcaaccgatagaaatatcggtccggttgaaaagagcatcggtctgaaggatgcactccgggatagggcagtcccggctcttgctgttaagagcaagtccttcagcaagtcgaaagacacgatgtgagcctatttt

SEQ ID NO: 381 - RNA, 140 nt, synthetic construct
  source 1..140: mol_type = other RNA
gtttcaatcaaccgatagaaatatcggtccggttgaaaagagcatcggtctgaaggatgcactccgggatagggcagtcccggctcttgctgtaaggcaagtccttcagcaagtcgaaagacacgatgtgagcctatttt

SEQ ID NO: 382 - RNA, 138 nt, synthetic construct
  source 1..138: mol_type = other RNA
gtttcaatcaaccgatagaaatatcggtccggttgaaaagagcatcggtctgaaggatgcactccgggatagggcagtcccggctcttgctgaagcaagtccttcagcaagtcgaaagacacgatgtgagcctatttt

SEQ ID NO: 383 - RNA, 136 nt, synthetic construct
  source 1..136: mol_type = other RNA
gtttcaatcaaccgatagaaatatcggtccggttgaaaagagcatcggtctgaaggatgcactccgggatagggcagtcccggctcttgctaagaagtccttcagcaagtcgaaagacacgatgtgagcctatttt

SEQ ID NO: 384 - RNA, 133 nt, synthetic construct
  source 1..133: mol_type = other RNA
gtttcaatcaaccgatagaaatatcggtccggttgaaaagagcatcggtctgaaggatgcactccgggatagggcagtcccggctcttgctaagtccttcagcaagtcgaaagacacgatgtgagcctatttt

SEQ ID NO: 385 - RNA, 158 nt, synthetic construct
  source 1..158: mol_type = other RNA
gtttcaatcaaccgatagaaatatcggtccggttgaaaagagcatcggtctgaaggatgcactccgggatagggcagtcccggctcttgctgtttccgaggtaagacctcggaagcaagtccttcagcaagtcgaaagacacgatgtgagcctatttt

SEQ ID NO: 386 - RNA, 158 nt, synthetic construct
  source 1..158: mol_type = other RNA
gtttcaatcaaccgatagaaatatcggtccggttgaaaagagcatcggtctgaaggatgcactccgggatagggcagtcccggctcttgctgtttccccggtaagaccggggaagcaagtccttcagcaagtcgaaagacacgatgtgagcctatttt

SEQ ID NO: 387 - RNA, 158 nt, synthetic construct
  source 1..158: mol_type = other RNA
gtttcaatcaaccgatagaaatatcggtccggttgaaaagagcatcggtctgaaggatgcactccgggatagggcagtcccggctcttgctgtttccgcggtaagacctcggaagcaagtccttcagcaagtcgaaagacacgatgtgagcctatttt

SEQ ID NO: 388 - RNA, 158 nt, synthetic construct
  source 1..158: mol_type = other RNA
gtttcaatcaaccgatagaaatatcggtccggttgaaaagagcatcggtctgaaggatgcactccgggatagggcagtcccggctcttgctgtttcccaggtaagacctcggaagcaagtccttcagcaagtcgaaagacacgatgtgagcctatttt

SEQ ID NO: 389 - RNA, 158 nt, synthetic construct
  source 1..158: mol_type = other RNA
gtttcaatcaaccgatagaaatatcggtccggttgaaaagagcatcggtctgaaggatgcactccgggatagggcagtcccggctcttgctgtttccccggtaagaccgcggaagcaagtccttcagcaagtcgaaagacacgatgtgagcctatttt

SEQ ID NO: 390 - RNA, 158 nt, synthetic construct
  source 1..158: mol_type = other RNA
gtttcaatcaaccgatagaaatatcggtccggttgaaaagagcatcggtctgaaggatgcactccgggatagggcagtcccggctcttgctgtttccccggtaagacctgggaagcaagtccttcagcaagtcgaaagacacgatgtgagcctatttt

SEQ ID NO: 391 - RNA, 155 nt, synthetic construct
  source 1..155: mol_type = other RNA
gtttcaatcaaccgatagaaatatcggtccggttgaaaagagcatcggtctgaaggatgcactccgggatagggcagtcccggctcttgctgtttccggtaagaccgggaagcaagtccttcagcaagtcgaaagacacgatgtgagcctatttt

SEQ ID NO: 392 - RNA, 154 nt, synthetic construct
  source 1..154: mol_type = other RNA
gtttcaatcaaccgatagaaatatcggtccggttgaaaagagcatcggtctgaaggatgcactccgggatagggcagtcccggctcttgctgtttccggtaagaccggaagcaagtccttcagcaagtcgaaagacacgatgtgagcctatttt

SEQ ID NO: 393 - RNA, 159 nt, synthetic construct
  source 1..159: mol_type = other RNA
gtttcaatcaaccgatagaaatatcggtccggttgaaaagagcatcggtctgaaggatgcactccgggatagggcagtcccggctcttgctgtttccccggtgaaaacctcggaagcaagtccttcagcaagtcgaaagacacgatgtgagcctatttt

SEQ ID NO: 394 - RNA, 157 nt, synthetic construct
  source 1..157: mol_type = other RNA
gtttcaatcaaccgatagaaatatcggtccggttgaaaagagcatcggtctgaaggatgcactccgggatagggcagtcccggctcttgctgtttccccgggaaacctcggaagcaagtccttcagcaagtcgaaagacacgatgtgagcctatttt

SEQ ID NO: 395 - RNA, 155 nt, synthetic construct
  source 1..155: mol_type = other RNA
gtttcaatcaaccgatagaaatatcggtccggttgaaaagagcatcggtctgaaggatgcactccgggatagggcagtcccggctcttgctgtttccccggaaactcggaagcaagtccttcagcaagtcgaaagacacgatgtgagcctatttt

SEQ ID NO: 396 - RNA, 153 nt, synthetic construct
  source 1..153: mol_type = other RNA
gtttcaatcaaccgatagaaatatcggtccggttgaaaagagcatcggtctgaaggatgcactccgggatagggcagtcccggctcttgctgtttccccgaaatcggaagcaagtccttcagcaagtcgaaagacacgatgtgagcctatttt

SEQ ID NO: 397 - RNA, 158 nt, synthetic construct
  source 1..158: mol_type = other RNA
gtttcaaccaaccgatagaaatatcggtccggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 398 - RNA, 158 nt, synthetic construct
  source 1..158: mol_type = other RNA
gtttcaatcaaccgatagaaatatcggtccagttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 399 - RNA, 158 nt, synthetic construct
  source 1..158: mol_type = other RNA
gtttcaagcaaccgatagaaatatcggtcccgttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 400 - RNA, 158 nt, synthetic construct
  source 1..158: mol_type = other RNA
gtttcaatcaaccgatagaaatatcggtccggttgaaaagaacatcggtctgaaggatgcactccgggatagggcagtcccggctcttgctgtttccccggtaagacctcggaagcaagtccttcagcaagtcgaaagacacgatgtgagcctatttt

SEQ ID NO: 401 - RNA, 158 nt, synthetic construct
  source 1..158: mol_type = other RNA
gtttcaatcaaccgatagaaatatcggtccggttgaaaagagcatcggtctgaaggatgcactccgggatagggcagtcccggctcttgctgtttccccggtaagacctcggaagcaagtccttcagcaagtcgaaagacacgatgcgagcctatttt

SEQ ID NO: 402 - RNA, 158 nt, synthetic construct
  source 1..158: mol_type = other RNA
gtttcaatcaaccgatagaaatatcggtccggttgaaaagaccatcggtctgaaggatgcactccgggatagggcagtcccggctcttgctgtttccccggtaagacctcggaagcaagtccttcagcaagtcgaaagacacgatgggagcctatttt

SEQ ID NO: 403 - RNA, 158 nt, synthetic construct
  source 1..158: mol_type = other RNA
gtttcaatcaaccgatagaaatatcggtccggttgaaaagagcatcggtctgaaggacgcactccgggatagggcagtcccggctcttgctgtttccccggtaagacctcggaagcaagtccttcagcaagtcgaaagacacgatgtgagcctatttt

SEQ ID NO: 404 - RNA, 158 nt, synthetic construct
  source 1..158: mol_type = other RNA
gtttcaatcaaccgatagaaatatcggtccggttgaaaagagcatcggtctgaaggatgcactccgggatagggcagtcccggctcttgctgtttccccggtaagacctcggaagcaaatccttcagcaagtcgaaagacacgatgtgagcctatttt

SEQ ID NO: 405 - RNA, 158 nt, synthetic construct
  source 1..158: mol_type = other RNA
gtttcaatcaaccgatagaaatatcggtccggttgaaaagagcatcggtctgaaggaggcactccgggatagggcagtcccggctcttgctgtttccccggtaagacctcggaagcaactccttcagcaagtcgaaagacacgatgtgagcctatttt

SEQ ID NO: 406 - RNA, 158 nt, synthetic construct
  source 1..158: mol_type = other RNA
gtttcaatcaaccgatagaaatatcggtccggttgaaaagagcatcggtctgaaggatgcactccgggacagggcagtcccggctcttgctgtttccccggtaagacctcggaagcaagtccttcagcaagtcgaaagacacgatgtgagcctatttt

SEQ ID NO: 407 - RNA, 158 nt, synthetic construct
  source 1..158: mol_type = other RNA
gtttcaatcaaccgatagaaatatcggtccggttgaaaagagcatcggtctgaaggatgcactccgggatagggcaatcccggctcttgctgtttccccggtaagacctcggaagcaagtccttcagcaagtcgaaagacacgatgtgagcctatttt

SEQ ID NO: 408 - RNA, 158 nt, synthetic construct
  source 1..158: mol_type = other RNA
gtttcaatcaaccgatagaaatatcggtccggttgaaaagagcatcggtctgaaggatgcactccgggagagggcactcccggctcttgctgtttccccggtaagacctcggaagcaagtccttcagcaagtcgaaagacacgatgtgagcctatttt

SEQ ID NO: 409 - RNA, 158 nt, synthetic construct
  source 1..158: mol_type = other RNA
gtttcaatcaaccgatagaaatatcggtccggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 410 - RNA, 117 nt, synthetic construct
  source 1..117: mol_type = other RNA
gtttcaatcaaccgagaaatcggtccggttgaaaagagcatcggctgaaggatgcactccgggatagggcagtcccggctcttgctaagtccttcagcttacgatgtgagccttttt

SEQ ID NO: 411 - RNA, 177 nt, synthetic construct
  source 1..177: mol_type = other RNA
gctcgtgggtttgtggttgccgtttcaatcaaccgatgaaaatcggtccggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 412 - RNA, 175 nt, synthetic construct
  source 1..175: mol_type = other RNA
gctcgtgggtttgtggttgccgtttcaatcaaccgagaaatcggtccggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 413 - RNA, 173 nt, synthetic construct
  source 1..173: mol_type = other RNA
gctcgtgggtttgtggttgccgtttcaatcaaccggaaacggtccggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 414 - RNA, 171 nt, synthetic construct
  source 1..171: mol_type = other RNA
gctcgtgggtttgtggttgccgtttcaatcaaccgaaaggtccggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 415 - RNA, 169 nt, synthetic construct
  source 1..169: mol_type = other RNA
gctcgtgggtttgtggttgccgtttcaatcaacgaaagtccggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 416 - RNA, 167 nt, synthetic construct
  source 1..167: mol_type = other RNA
gctcgtgggtttgtggttgccgtttcaatcaagaaatccggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 417 - RNA, 163 nt, synthetic construct
  source 1..163: mol_type = other RNA
gctcgtgggtttgtggttgccgtttcaatcaatccggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 418 - RNA, 162 nt, synthetic construct
  source 1..162: mol_type = other RNA
gctcgtgggtttgtggttgccgtttcaatcgaaaggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 419 - RNA, 160 nt, synthetic construct
  source 1..160: mol_type = other RNA
gctcgtgggtttgtggttgccgtttcaatgaaagttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 420 - RNA, 158 nt, synthetic construct
  source 1..158: mol_type = other RNA
gctcgtgggtttgtggttgccgtttcaagaaattgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 421 - RNA, 156 nt, synthetic construct
  source 1..156: mol_type = other RNA
gctcgtgggtttgtggttgccgtttcagaaatgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 422 - RNA, 175 nt, synthetic construct
  source 1..175: mol_type = other RNA
gctcgtgggtttgtggttgccgtttcaaaaccgatagaaatatcggtccttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 423 - RNA, 174 nt, synthetic construct
  source 1..174: mol_type = other RNA
gctcgtgggtttgtggttgccgtttcaaaccgatagaaatatcggtccttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 424 - RNA, 176 nt, synthetic construct
  source 1..176: mol_type = other RNA
gctcgtgggtttgtggttgccgtttcaaaccgatagaaatatcggtccggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 425 - RNA, 177 nt, synthetic construct
  source 1..177: mol_type = other RNA
gctcgtgggtttgtggttgccgtttcaaaccgatagaaatatcggtcctgattgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 426 - RNA, 173 nt, synthetic construct
  source 1..173: mol_type = other RNA
gctcgtgggtttgtggttgccgtttcaaaaccgatgaaaatcggtccttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 427 - RNA, 172 nt, synthetic construct
  source 1..172: mol_type = other RNA
gctcgtgggtttgtggttgccgtttcaaaccgatgaaaatcggtccttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 428 - RNA, 174 nt, synthetic construct
  source 1..174: mol_type = other RNA
gctcgtgggtttgtggttgccgtttcaaaccgatgaaaatcggtccggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 429 - RNA, 175 nt, synthetic construct
  source 1..175: mol_type = other RNA
gctcgtgggtttgtggttgccgtttcaaaccgatgaaaatcggtcctgattgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 430 - RNA, 178 nt, synthetic construct
  source 1..178: mol_type = other RNA
gctcgtgggtttgtggttgccgtttcaatcaaccgatagaaatatcggttggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 431 - RNA, 179 nt, synthetic construct
  source 1..179: mol_type = other RNA
gctcgtgggtttgtggttgccgtttcaatcgaccgatagaaatatcggtccggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 432 - RNA, 179 nt, synthetic construct
  source 1..179: mol_type = other RNA
gctcgtgggtttgtggttgccgtttcaatcaaccgatagaaatatcggttcggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 433 - RNA, 179 nt, synthetic construct
  source 1..179: mol_type = other RNA
gctcgtgggtttgtggttgccgtttcaatcaaccgatagaaatatcggtctggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 434 - RNA, 178 nt, synthetic construct
  source 1..178: mol_type = other RNA
gctcgtgggtttgtggttgccgtttcaatcgaccgatagaaatatcggtcggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 435 - RNA, 178 nt, synthetic construct
  source 1..178: mol_type = other RNA
gctcgtgggtttgtggttgcctttcaatcaaccgatagaaatatcggtccggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 436 - RNA, 177 nt, synthetic construct
  source 1..177: mol_type = other RNA
gctcgtgggtttgtggttgccgtttcaatcaaccgatagaaatatcggtccggttgaaatgcatcggtctgaaggatgcactccgggatagggcagtcccggctcttgctgtttccccggtaagacctcggaagcaagtccttcagcaagtcgaaagacacgatgtgagcctatttt

SEQ ID NO: 437 - RNA, 178 nt, synthetic construct
  source 1..178: mol_type = other RNA
gctcgtgggtttgtggttgccgtttcaatcaaccgatagaaatatcggtccggttgaaaagagcatcggtctgaaggatgcactccgggatagggcagtcccggctcttgctgtttccccggtaagacctcggaagcaagtccttcagcaagtcgaaagacacgatgtgagcctattt

SEQ ID NO: 438 - RNA, 177 nt, synthetic construct
  source 1..177: mol_type = other RNA
gctcgtgggtttgtggttgccgtttcaatcaaccgatagaaatatcggtccggttgaaaagagcatcggtctgaaggatgcactccgggatagggcagtcccggctcttgctgtttccccggtaagacctcggaagcaagtccttcagcaagtcgaaagacacgatgtgagcctatt

SEQ ID NO: 439 - RNA, 176 nt, synthetic construct
  source 1..176: mol_type = other RNA
gctcgtgggtttgtggttgccgtttcaatcaaccgatagaaatatcggtccggttgaaaagagcatcggtctgaaggatgcactccgggatagggcagtcccggctcttgctgtttccccggtaagacctcggaagcaagtccttcagcaagtcgaaagacacgatgtgagcctat

SEQ ID NO: 440 - RNA, 175 nt, synthetic construct
  source 1..175: mol_type = other RNA
gctcgtgggtttgtggttgccgtttcaatcaaccgatagaaatatcggtccggttgaaaagagcatcggtctgaaggatgcactccgggatagggcagtcccggctcttgctgtttccccggtaagacctcggaagcaagtccttcagcaagtcgaaagacacgatgtgagccta

SEQ ID NO: 441 - RNA, 174 nt, synthetic construct
  source 1..174: mol_type = other RNA
gctcgtgggtttgtggttgccgtttcaatcaaccgatagaaatatcggtccggttgaaaagagcatcggtctgaaggatgcactccgggatagggcagtcccggctcttgctgtttccccggtaagacctcggaagcaagtccttcagcaagtcgaaagacacgatgtgagcct

SEQ ID NO: 442 - RNA, 173 nt, synthetic construct
  source 1..173: mol_type = other RNA
gctcgtgggtttgtggttgccgtttcaatcaaccgatagaaatatcggtccggttgaaaagagcatcggtctgaaggatgcactccgggatagggcagtcccggctcttgctgtttccccggtaagacctcggaagcaagtccttcagcaagtcgaaagacacgatgtgagcc

SEQ ID NO: 443 - RNA, 172 nt, synthetic construct
  source 1..172: mol_type = other RNA
gctcgtgggtttgtggttgccgtttcaatcaaccgatagaaatatcggtccggttgaaaagagcatcggtctgaaggatgcactccgggatagggcagtcccggctcttgctgtttccccggtaagacctcggaagcaagtccttcagcaagtcgaaagacacgatgtgagc

SEQ ID NO: 444 - RNA, 171 nt, synthetic construct
  source 1..171: mol_type = other RNA
gctcgtgggtttgtggttgccgtttcaatcaaccgatagaaatatcggtccggttgaaaagagcatcggtctgaaggatgcactccgggatagggcagtcccggctcttgctgtttccccggtaagacctcggaagcaagtccttcagcaagtcgaaagacacgatgtgag

SEQ ID NO: 445 - RNA, 170 nt, synthetic construct
  source 1..170: mol_type = other RNA
gctcgtgggtttgtggttgccgtttcaatcaaccgatagaaatatcggtccggttgaaaagagcatcggtctgaaggatgcactccgggatagggcagtcccggctcttgctgtttccccggtaagacctcggaagcaagtccttcagcaagtcgaaagacacgatgtga

SEQ ID NO: 446 - RNA, 169 nt, synthetic construct
  source 1..169: mol_type = other RNA
gctcgtgggtttgtggttgccgtttcaatcaaccgatagaaatatcggtccggttgaaaagagcatcggtctgaaggatgcactccgggatagggcagtcccggctcttgctgtttccccggtaagacctcggaagcaagtccttcagcaagtcgaaagacacgatgtg

SEQ ID NO: 447 - RNA, 168 nt, synthetic construct
  source 1..168: mol_type = other RNA
gctcgtgggtttgtggttgccgtttcaatcaaccgatagaaatatcggtccggttgaaaagagcatcggtctgaaggatgcactccgggatagggcagtcccggctcttgctgtttccccggtaagacctcggaagcaagtccttcagcaagtcgaaagacacgatgt

SEQ ID NO: 448 - RNA, 178 nt, synthetic construct
  source 1..178: mol_type = other RNA
gctcgtgggtttgtggttgccgtttcaatcaaccgatagaaatatcggtccggttgaaaagagcatcggctgaaggatgcactccgggatagggcagtcccggctcttgctgtttccccggtaagacctcggaagcaagtccttcagcaagtcgaaagacacgatgtgagcctatttt

SEQ ID NO: 449 - RNA, 177 nt, synthetic construct
  source 1..177: mol_type = other RNA
gctcgtgggtttgtggttgccgtttcaatcaaccgatagaaatatcggtccggttgaaaagagcatcggtctgaaggatgcactccgggatagggcagtcccggctcttgctgtttccccggtaagacctcggaagcaagtccttcagcaagtgaaaacacgatgtgagcctatttt

SEQ ID NO: 450 - RNA, 175 nt, synthetic construct
  source 1..175: mol_type = other RNA
gctcgtgggtttgtggttgccgtttcaatcaaccgatagaaatatcggtccggttgaaaagagcatcggtctgaaggatgcactccgggatagggcagtcccggctcttgctgtttccccggtaagacctcggaagcaagtccttcagcaaggaaacacgatgtgagcctatttt

SEQ ID NO: 451 - RNA, 173 nt, synthetic construct
  source 1..173: mol_type = other RNA
gctcgtgggtttgtggttgccgtttcaatcaaccgatagaaatatcggtccggttgaaaagagcatcggtctgaaggatgcactccgggatagggcagtcccggctcttgctgtttccccggtaagacctcggaagcaagtccttcagcaagaaaacgatgtgagcctatttt

SEQ ID NO: 452 - RNA, 172 nt, synthetic construct
  source 1..172: mol_type = other RNA
gctcgtgggtttgtggttgccgtttcaatcaaccgatagaaatatcggtccggttgaaaagagcatcggtctgaaggatgcactccgggatagggcagtcccggctcttgctgtttccccggtaagacctcggaagcaagtccttcagcagaaaacgatgtgagcctatttt

SEQ ID NO: 453 - RNA, 166 nt, synthetic construct
  source 1..166: mol_type = other RNA
gctcgtgggtttgtggttgccgtttcaatcaaccgatagaaatatcggtccggttgaaaagagcatcggctgaaggatgcactccgggatagggcagtcccggctcttgctgtttccccggtaagacctcggaagcaagtccttcagcacgatgtgagcctatttt

SEQ ID NO: 454 - RNA, 168 nt, synthetic construct
  source 1..168: mol_type = other RNA
gctcgtgggtttgtggttgccgtttcaatcaaccgatagaaatatcggtccggttgaaaagagcatcggctgaaggatgcactccgggatagggcagtcccggctcttgctgtttccccggtaagacctcggaagcaagtccttcagcttacgatgtgagcctatttt

SEQ ID NO: 455 - RNA, 169 nt, synthetic construct
  source 1..169: mol_type = other RNA
gctcgtgggtttgtggttgccgtttcaatcaaccgatagaaatatcggtccggttgaaaagagcatcggctgaaggatgcactccgggatagggcagtcccggctcttgctgtttccccggtaagacctcggaagcaagtccttcagcgagccgatgtgagcctatttt

SEQ ID NO: 456 - RNA, 170 nt, synthetic construct
  source 1..170: mol_type = other RNA
gctcgtgggtttgtggttgccgtttcaatcaaccgatagaaatatcggtccggttgaaaagagcatcgagctgaaggatgcactccgggatagggcagtcccggctcttgctgtttccccggtaagacctcggaagcaagtccttcagcgagccgatgtgagcctatttt

SEQ ID NO: 457 - RNA, 166 nt, synthetic construct
  source 1..166: mol_type = other RNA
gctcgtgggtttgtggttgccgtttcaatcaaccgatagaaatatcggtccggttgaaaagagcatcggtctgaaggatgcactccgggatagggcagtcccggctcttgctgtttccccggtaagacctcggaagcaagtccttcagccgatgtgagcctatttt

SEQ ID NO: 458 - RNA, 165 nt, synthetic construct
  source 1..165: mol_type = other RNA
gctcgtgggtttgtggttgccgtttcaatcaaccgatagaaatatcggtccggttgaaaagagcatcggctgaaggatgcactccgggatagggcagtcccggctcttgctgtttccccggtaagacctcggaagcaagtccttcagccgatgtgagcctatttt

SEQ ID NO: 459 - RNA, 177 nt, synthetic construct
  source 1..177: mol_type = other RNA
gctcgtgggtttgtggttgccgtttcaatcaaccgatagaaatatcggtccggttgaaaagagcatcggtctgaaggatgcactccgggaagggcatcccggctcttgctgtttccccggtaagacctcggaagcaagtccttcagcaagtcgaaagacacgatgtgagcctatttt

SEQ ID NO: 460 - RNA, 175 nt, synthetic construct
  source 1..175: mol_type = other RNA
gctcgtgggtttgtggttgccgtttcaatcaaccgatagaaatatcggtccggttgaaaagagcatcggtctgaaggatgcactccgggagggcacccggctcttgctgtttccccggtaagacctcggaagcaagtccttcagcaagtcgaaagacacgatgtgagcctatttt

SEQ ID NO: 461 - RNA, 173 nt, synthetic construct
  source 1..173: mol_type = other RNA
gctcgtgggtttgtggttgccgtttcaatcaaccgatagaaatatcggtccggttgaaaagagcatcggtctgaaggatgcactccggagggcaccggctcttgctgtttccccggtaagacctcggaagcaagtccttcagcaagtcgaaagacacgatgtgagcctatttt

SEQ ID NO: 462 - RNA, 171 nt, synthetic construct
  source 1..171: mol_type = other RNA
gctcgtgggtttgtggttgccgtttcaatcaaccgatagaaatatcggtccggttgaaaagagcatcggtctgaaggatgcactccgagggcacggctcttgctgtttccccggtaagacctcggaagcaagtccttcagcaagtcgaaagacacgatgtgagcctatttt

SEQ ID NO: 463 - RNA, 169 nt, synthetic construct
  source 1..169: mol_type = other RNA
gctcgtgggtttgtggttgccgtttcaatcaaccgatagaaatatcggtccggttgaaaagagcatcggtctgaaggatgcactccagggcaggctcttgctgtttccccggtaagacctcggaagcaagtccttcagcaagtcgaaagacacgatgtgagcctatttt

SEQ ID NO: 464 - RNA, 167 nt, synthetic construct
  source 1..167: mol_type = other RNA
gctcgtgggtttgtggttgccgtttcaatcaaccgatagaaatatcggtccggttgaaaagagcatcggtctgaaggatgcactcagggcagctcttgctgtttccccggtaagacctcggaagcaagtccttcagcaagtcgaaagacacgatgtgagcctatttt

SEQ ID NO: 465 - RNA, 165 nt, synthetic construct
  source 1..165: mol_type = other RNA
gctcgtgggtttgtggttgccgtttcaatcaaccgatagaaatatcggtccggttgaaaagagcatcggtctgaaggatgcactagggcactcttgctgtttccccggtaagacctcggaagcaagtccttcagcaagtcgaaagacacgatgtgagcctatttt

SEQ ID NO: 466 - RNA, 159 nt, synthetic construct
  source 1..159: mol_type = other RNA
gctcgtgggtttgtggttgccgtttcaatcaaccgatagaaatatcggtccggttgaaaagagcatcggtctgaaggatgcaagggcatgctgtttccccggtaagacctcggaagcaagtccttcagcaagtcgaaagacacgatgtgagcctatttt

SEQ ID NO: 467 - RNA, 157 nt, synthetic construct
  source 1..157: mol_type = other RNA
gctcgtgggtttgtggttgccgtttcaatcaaccgatagaaatatcggtccggttgaaaagagcatcggtctgaaggatgcagggcagctgtttccccggtaagacctcggaagcaagtccttcagcaagtcgaaagacacgatgtgagcctatttt

SEQ ID NO: 468 - RNA, 155 nt, synthetic construct
  source 1..155: mol_type = other RNA
gctcgtgggtttgtggttgccgtttcaatcaaccgatagaaatatcggtccggttgaaaagagcatcggtctgaaggatgagggcactgtttccccggtaagacctcggaagcaagtccttcagcaagtcgaaagacacgatgtgagcctatttt

SEQ ID NO: 469 - RNA, 149 nt, synthetic construct
  source 1..149: mol_type = other RNA
gctcgtgggtttgtggttgccgtttcaatcaaccgatagaaatatcggtccggttgaaaagagcatcggtctgaaggatgctgtttccccggtaagacctcggaagcaagtccttcagcaagtcgaaagacacgatgtgagcctatttt

SEQ ID NO: 470 - RNA, 177 nt, synthetic construct
  source 1..177: mol_type = other RNA
gctcgtgggtttgtggttgccgtttcaatcaaccgatagaaatatcggtccggttgaaaagagcatcggtctgaaggatgcactccgggatagggcagtcccggctcttgctgtttccccggaagcctcggaagcaagtccttcagcaagtcgaaagacacgatgtgagcctatttt

SEQ ID NO: 471 - RNA, 175 nt, synthetic construct
  source 1..175: mol_type = other RNA
gctcgtgggtttgtggttgccgtttcaatcaaccgatagaaatatcggtccggttgaaaagagcatcggtctgaaggatgcactccgggatagggcagtcccggctcttgctgtttccccgaagctcggaagcaagtccttcagcaagtcgaaagacacgatgtgagcctatttt

SEQ ID NO: 472 - RNA, 173 nt, synthetic construct
  source 1..173: mol_type = other RNA
gctcgtgggtttgtggttgccgtttcaatcaaccgatagaaatatcggtccggttgaaaagagcatcggtctgaaggatgcactccgggatagggcagtcccggctcttgctgtttccccaagtcggaagcaagtccttcagcaagtcgaaagacacgatgtgagcctatttt

SEQ ID NO: 473 - RNA, 169 nt, synthetic construct
  source 1..169: mol_type = other RNA
gctcgtgggtttgtggttgccgtttcaatcaaccgatagaaatatcggtccggttgaaaagagcatcggtctgaaggatgcactccgggatagggcagtcccggctcttgctgtttccaagggaagcaagtccttcagcaagtcgaaagacacgatgtgagcctatttt

SEQ ID NO: 474 - RNA, 167 nt, synthetic construct
  source 1..167: mol_type = other RNA
gctcgtgggtttgtggttgccgtttcaatcaaccgatagaaatatcggtccggttgaaaagagcatcggtctgaaggatgcactccgggatagggcagtcccggctcttgctgtttcaaggaagcaagtccttcagcaagtcgaaagacacgatgtgagcctatttt

SEQ ID NO: 475 - RNA, 165 nt, synthetic construct
  source 1..165: mol_type = other RNA
gctcgtgggtttgtggttgccgtttcaatcaaccgatagaaatatcggtccggttgaaaagagcatcggtctgaaggatgcactccgggatagggcagtcccggctcttgctgtttaagaagcaagtccttcagcaagtcgaaagacacgatgtgagcctatttt

SEQ ID NO: 476 - RNA, 163 nt, synthetic construct
  source 1..163: mol_type = other RNA
gctcgtgggtttgtggttgccgtttcaatcaaccgatagaaatatcggtccggttgaaaagagcatcggtctgaaggatgcactccgggatagggcagtcccggctcttgctgttaagagcaagtccttcagcaagtcgaaagacacgatgtgagcctatttt

SEQ ID NO: 477 - RNA, 161 nt, synthetic construct
  source 1..161: mol_type = other RNA
gctcgtgggtttgtggttgccgtttcaatcaaccgatagaaatatcggtccggttgaaaagagcatcggtctgaaggatgcactccgggatagggcagtcccggctcttgctgtaaggcaagtccttcagcaagtcgaaagacacgatgtgagcctatttt

SEQ ID NO: 478 - RNA, 159 nt, synthetic construct
  source 1..159: mol_type = other RNA
gctcgtgggtttgtggttgccgtttcaatcaaccgatagaaatatcggtccggttgaaaagagcatcggtctgaaggatgcactccgggatagggcagtcccggctcttgctgaagcaagtccttcagcaagtcgaaagacacgatgtgagcctatttt

SEQ ID NO: 479 - RNA, 157 nt, synthetic construct
  source 1..157: mol_type = other RNA
gctcgtgggtttgtggttgccgtttcaatcaaccgatagaaatatcggtccggttgaaaagagcatcggtctgaaggatgcactccgggatagggcagtcccggctcttgctaagaagtccttcagcaagtcgaaagacacgatgtgagcctatttt

SEQ ID NO: 480 - RNA, 154 nt, synthetic construct
  source 1..154: mol_type = other RNA
gctcgtgggtttgtggttgccgtttcaatcaaccgatagaaatatcggtccggttgaaaagagcatcggtctgaaggatgcactccgggatagggcagtcccggctcttgctaagtccttcagcaagtcgaaagacacgatgtgagcctatttt

SEQ ID NO: 481 - RNA, 179 nt, synthetic construct
  source 1..179: mol_type = other RNA
gctcgtgggtttgtggttgccgtttcaatcaaccgatagaaatatcggtccggttgaaaagagcatcggtctgaaggatgcactccgggatagggcagtcccggctcttgctgtttccgaggtaagacctcggaagcaagtccttcagcaagtcgaaagacacgatgtgagcctatttt

SEQ ID NO: 482 - RNA, 179 nt, synthetic construct
  source 1..179: mol_type = other RNA
gctcgtgggtttgtggttgccgtttcaatcaaccgatagaaatatcggtccggttgaaaagagcatcggtctgaaggatgcactccgggatagggcagtcccggctcttgctgtttccccggtaagaccggggaagcaagtccttcagcaagtcgaaagacacgatgtgagcctatttt

SEQ ID NO: 483 - RNA, 179 nt, synthetic construct
  source 1..179: mol_type = other RNA
gctcgtgggtttgtggttgccgtttcaatcaaccgatagaaatatcggtccggttgaaaagagcatcggtctgaaggatgcactccgggatagggcagtcccggctcttgctgtttccgcggtaagacctcggaagcaagtccttcagcaagtcgaaagacacgatgtgagcctatttt

SEQ ID NO: 484 - RNA, 179 nt, synthetic construct
  source 1..179: mol_type = other RNA
gctcgtgggtttgtggttgccgtttcaatcaaccgatagaaatatcggtccggttgaaaagagcatcggtctgaaggatgcactccgggatagggcagtcccggctcttgctgtttcccaggtaagacctcggaagcaagtccttcagcaagtcgaaagacacgatgtgagcctatttt

SEQ ID NO: 485 - RNA, 179 nt, synthetic construct
  source 1..179: mol_type = other RNA
gctcgtgggtttgtggttgccgtttcaatcaaccgatagaaatatcggtccggttgaaaagagcatcggtctgaaggatgcactccgggatagggcagtcccggctcttgctgtttccccggtaagaccgcggaagcaagtccttcagcaagtcgaaagacacgatgtgagcctatttt

SEQ ID NO: 486 - RNA, 179 nt, synthetic construct
  source 1..179: mol_type = other RNA
gctcgtgggtttgtggttgccgtttcaatcaaccgatagaaatatcggtccggttgaaaagagcatcggtctgaaggatgcactccgggatagggcagtcccggctcttgctgtttccccggtaagacctgggaagcaagtccttcagcaagtcgaaagacacgatgtgagcctatttt

SEQ ID NO: 487 - RNA, 176 nt, synthetic construct
  source 1..176: mol_type = other RNA
gctcgtgggtttgtggttgccgtttcaatcaaccgatagaaatatcggtccggttgaaaagagcatcggtctgaaggatgcactccgggatagggcagtcccggctcttgctgtttccggtaagaccgggaagcaagtccttcagcaagtcgaaagacacgatgtgagcctatttt

SEQ ID NO: 488 - RNA, 175 nt, synthetic construct
  source 1..175: mol_type = other RNA
gctcgtgggtttgtggttgccgtttcaatcaaccgatagaaatatcggtccggttgaaaagagcatcggtctgaaggatgcactccgggatagggcagtcccggctcttgctgtttccggtaagaccggaagcaagtccttcagcaagtcgaaagacacgatgtgagcctatttt

SEQ ID NO: 489 - RNA, 180 nt, synthetic construct
  source 1..180: mol_type = other RNA
gctcgtgggtttgtggttgccgtttcaatcaaccgatagaaatatcggtccggttgaaaagagcatcggtctgaaggatgcactccgggatagggcagtcccggctcttgctgtttccccggtgaaaacctcggaagcaagtccttcagcaagtcgaaagacacgatgtgagcctatttt

SEQ ID NO: 490 - RNA, 178 nt, synthetic construct
  source 1..178: mol_type = other RNA
gctcgtgggtttgtggttgccgtttcaatcaaccgatagaaatatcggtccggttgaaaagagcatcggtctgaaggatgcactccgggatagggcagtcccggctcttgctgtttccccgggaaacctcggaagcaagtccttcagcaagtcgaaagacacgatgtgagcctatttt

SEQ ID NO: 491 - RNA, 176 nt, synthetic construct
  source 1..176: mol_type = other RNA
gctcgtgggtttgtggttgccgtttcaatcaaccgatagaaatatcggtccggttgaaaagagcatcggtctgaaggatgcactccgggatagggcagtcccggctcttgctgtttccccggaaactcggaagcaagtccttcagcaagtcgaaagacacgatgtgagcctatttt

SEQ ID NO: 492 - RNA, 174 nt, synthetic construct
  source 1..174: mol_type = other RNA
gctcgtgggtttgtggttgccgtttcaatcaaccgatagaaatatcggtccggttgaaaagagcatcggtctgaaggatgcactccgggatagggcagtcccggctcttgctgtttccccgaaatcggaagcaagtccttcagcaagtcgaaagacacgatgtgagcctatttt

SEQ ID NO: 493 - RNA, 179 nt, synthetic construct
  source 1..179: mol_type = other RNA
gctcgtgggtttgtggttgccgtttcaaccaaccgatagaaatatcggtccggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 494 - RNA, 179 nt, synthetic construct
  source 1..179: mol_type = other RNA
gctcgtgggtttgtggttgccgtttcaatcaaccgatagaaatatcggtccagttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 495 - RNA, 179 nt, synthetic construct
  source 1..179: mol_type = other RNA
gctcgtgggtttgtggttgccgtttcaagcaaccgatagaaatatcggtcccgttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 496 - RNA, 179 nt, synthetic construct
  source 1..179: mol_type = other RNA
gctcgtgggtttgtggttgccgtttcaatcaaccgatagaaatatcggtccggttgaaaagaacatcggtctgaaggatgcactccgggatagggcagtcccggctcttgctgtttccccggtaagacctcggaagcaagtccttcagcaagtcgaaagacacgatgtgagcctatttt

SEQ ID NO: 497 - RNA, 179 nt, synthetic construct
  source 1..179: mol_type = other RNA
gctcgtgggtttgtggttgccgtttcaatcaaccgatagaaatatcggtccggttgaaaagagcatcggtctgaaggatgcactccgggatagggcagtcccggctcttgctgtttccccggtaagacctcggaagcaagtccttcagcaagtcgaaagacacgatgcgagcctatttt

SEQ ID NO: 498 - RNA, 179 nt, synthetic construct
  source 1..179: mol_type = other RNA
gctcgtgggtttgtggttgccgtttcaatcaaccgatagaaatatcggtccggttgaaaagaccatcggtctgaaggatgcactccgggatagggcagtcccggctcttgctgtttccccggtaagacctcggaagcaagtccttcagcaagtcgaaagacacgatgggagcctatttt

SEQ ID NO: 499 - RNA, 179 nt, synthetic construct
  source 1..179: mol_type = other RNA
gctcgtgggtttgtggttgccgtttcaatcaaccgatagaaatatcggtccggttgaaaagagcatcggtctgaaggacgcactccgggatagggcagtcccggctcttgctgtttccccggtaagacctcggaagcaagtccttcagcaagtcgaaagacacgatgtgagcctatttt

SEQ ID NO: 500 - RNA, 179 nt, synthetic construct
  source 1..179: mol_type = other RNA
gctcgtgggtttgtggttgccgtttcaatcaaccgatagaaatatcggtccggttgaaaagagcatcggtctgaaggatgcactccgggatagggcagtcccggctcttgctgtttccccggtaagacctcggaagcaaatccttcagcaagtcgaaagacacgatgtgagcctatttt

SEQ ID NO: 501 - RNA, 179 nt, synthetic construct
  source 1..179: mol_type = other RNA
gctcgtgggtttgtggttgccgtttcaatcaaccgatagaaatatcggtccggttgaaaagagcatcggtctgaaggaggcactccgggatagggcagtcccggctcttgctgtttccccggtaagacctcggaagcaactccttcagcaagtcgaaagacacgatgtgagcctatttt

SEQ ID NO: 502 - RNA, 179 nt, synthetic construct
  source 1..179: mol_type = other RNA
gctcgtgggtttgtggttgccgtttcaatcaaccgatagaaatatcggtccggttgaaaagagcatcggtctgaaggatgcactccgggacagggcagtcccggctcttgctgtttccccggtaagacctcggaagcaagtccttcagcaagtcgaaagacacgatgtgagcctatttt

SEQ ID NO: 503 - RNA, 179 nt, synthetic construct
  source 1..179: mol_type = other RNA
gctcgtgggtttgtggttgccgtttcaatcaaccgatagaaatatcggtccggttgaaaagagcatcggtctgaaggatgcactccgggatagggcaatcccggctcttgctgtttccccggtaagacctcggaagcaagtccttcagcaagtcgaaagacacgatgtgagcctatttt

SEQ ID NO: 504 - RNA, 179 nt, synthetic construct
  source 1..179: mol_type = other RNA
gctcgtgggtttgtggttgccgtttcaatcaaccgatagaaatatcggtccggttgaaaagagcatcggtctgaaggatgcactccgggagagggcactcccggctcttgctgtttccccggtaagacctcggaagcaagtccttcagcaagtcgaaagacacgatgtgagcctatttt

SEQ ID NO: 505 - RNA, 179 nt, synthetic construct
  source 1..179: mol_type = other RNA
gctcgtgggtttgtggttgccgtttcaatcaaccgatagaaatatcggtccggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 506 - RNA, 138 nt, synthetic construct
  source 1..138: mol_type = other RNA
gctcgtgggtttgtggttgccgtttcaatcaaccgagaaatcggtccggttgaaaagagcatcggctgaaggatgcactccgggatagggcagtcccggctcttgctaagtccttcagcttacgatgtgagccttttt

SEQ ID NO: 507 - protein, 7 aa, synthetic construct
NKKKSRR

SEQ ID NO: 508 - protein, 7 aa, synthetic construct
ANKKTSP

SEQ ID NO: 509 - DNA, 20 bp, unidentified
  source 1..20: mol_type = unassigned DNA
ctccaatgactagggtgggc

SEQ ID NO: 510 - DNA, 20 bp, unidentified
  source 1..20: mol_type = unassigned DNA
atgggctggctgaggcctgg

SEQ ID NO: 511 - DNA, 20 bp, unidentified
  source 1..20: mol_type = unassigned DNA
agacaaggcacttggttgat

SEQ ID NO: 512 - DNA, 20 bp, unidentified
  source 1..20: mol_type = unassigned DNA
ggacctccaggtgcagaagt

SEQ ID NO: 513 - DNA, 20 bp, unidentified
  source 1..20: mol_type = unassigned DNA
ctgtgaagagccagtggcca

SEQ ID NO: 514 - DNA, 20 bp, unidentified
  source 1..20: mol_type = unassigned DNA
ggacaaaggtcctcaaagac

SEQ ID NO: 515 - DNA, 20 bp, unidentified
  source 1..20: mol_type = unassigned DNA
gacttgggtgagcgatggac

SEQ ID NO: 516 - DNA, 20 bp, unidentified
  source 1..20: mol_type = unassigned DNA
ctccctatcagtgatagaga

SEQ ID NO: 517 - DNA, 20 bp, unidentified
  source 1..20: mol_type = unassigned DNA
agccacgtctacagcagggc

SEQ ID NO: 518 - DNA, 24 bp, unidentified
  source 1..24: mol_type = unassigned DNA
ttacagccacgtctacagcagggc

SEQ ID NO: 519 - DNA, 20 bp, unidentified
  source 1..20: mol_type = unassigned DNA
acagcagggctgcctcggac

SEQ ID NO: 520 - DNA, 24 bp, unidentified
  source 1..24: mol_type = unassigned DNA
gtctacagcagggctgcctcggac

SEQ ID NO: 521 - DNA, 20 bp, unidentified
  source 1..20: mol_type = unassigned DNA
gagggatcctgggagccctt

SEQ ID NO: 522 - DNA, 24 bp, unidentified
  source 1..24: mol_type = unassigned DNA
ctctgagggatcctgggagccctt

SEQ ID NO: 523 - DNA, 20 bp, unidentified
  source 1..20: mol_type = unassigned DNA
cccacacgcacacactcact

SEQ ID NO: 524 - DNA, 133 bp, synthetic construct
  source 1..133: mol_type = other DNA
gtttcaatcaaccgatagaaatatcggtccggttgaaaagagcatcggtctgaaggatgcactccgggatagggcagtcccggctcttgctaagtccttcagcaagtcgaaagacacgatgtgagcctatttt

SEQ ID NO: 525 - DNA, 133 bp, synthetic construct
  source 1..133: mol_type = other DNA
gcttcaatcaaccgatagaaatatcggtccggttgaagagagcatcggtctgaaggatgcactccgggatagggcagtcccggctcttgctaagtccttcagcaagtcgaaagacacgatgtgagcctatttt

SEQ ID NO: 526 - DNA, 133 bp, synthetic construct
  source 1..133: mol_type = other DNA
gtctcaatcaaccgatagaaatatcggtccggttgagaagagcatcggtctgaaggatgcactccgggatagggcagtcccggctcttgctaagtccttcagcaagtcgaaagacacgatgtgagcctatttt

SEQ ID NO: 527 - DNA, 133 bp, synthetic construct
  source 1..133: mol_type = other DNA
gtttcgatcaaccgatagaaatatcggtccggtcgaaaagagcatcggtctgaaggatgcactccgggatagggcagtcccggctcttgctaagtccttcagcaagtcgaaagacacgatgtgagcctatttt

SEQ ID NO: 528 - DNA, 133 bp, synthetic construct
  source 1..133: mol_type = other DNA
gtttcaatcagccgatagaaatatcggcccggttgaaaagagcatcggtctgaaggatgcactccgggatagggcagtcccggctcttgctaagtccttcagcaagtcgaaagacacgatgtgagcctatttt

SEQ ID NO: 529 - DNA, 133 bp, synthetic construct
  source 1..133: mol_type = other DNA
gtttcaatcacccgatagaaatatcgggccggttgaaaagagcatcggtctgaaggatgcactccgggatagggcagtcccggctcttgctaagtccttcagcaagtcgaaagacacgatgtgagcctatttt

SEQ ID NO: 530 - DNA, 131 bp, synthetic construct
  source 1..131: mol_type = other DNA
gtttcaatcaaccgatgaaaatcggtccggttgaaaagagcatcggtctgaaggatgcactccgggatagggcagtcccggctcttgctaagtccttcagcaagtcgaaagacacgatgtgagcctatttt

SEQ ID NO: 531 - DNA, 129 bp, synthetic construct
  source 1..129: mol_type = other DNA
gtttcaatcaaccgagaaatcggtccggttgaaaagagcatcggtctgaaggatgcactccgggatagggcagtcccggctcttgctaagtccttcagcaagtcgaaagacacgatgtgagcctatttt

SEQ ID NO: 532 - DNA, 132 bp, synthetic construct
  source 1..132: mol_type = other DNA
gtttcaatcaaccgatagaaatatcggtccggttgaaaagagcatcggctgaaggatgcactccgggatagggcagtcccggctcttgctaagtccttcagcaagtcgaaagacacgatgtgagcctatttt

SEQ ID NO: 533 - DNA, 131 bp, synthetic construct
  source 1..131: mol_type = other DNA
gtttcaatcaaccgatagaaatatcggtccggttgaaaagagcatcggtctgaaggatgcactccgggatagggcagtcccggctcttgctaagtccttcagcaagcgaaagcacgatgtgagcctatttt

SEQ ID NO: 534 - DNA, 133 bp, synthetic construct
  source 1..133: mol_type = other DNA
gcctcaatcaaccgatagaaatatcggtccggttgaggagagcatcggtctgaaggatgcactccgggatagggcagtcccggctcttgctaagtccttcagcaagtcgaaagacacgatgtgagcctatttt

SEQ ID NO: 535 - DNA, 133 bp, synthetic construct
  source 1..133: mol_type = other DNA
gcctcgatcaaccgatagaaatatcggtccggtcgaggagagcatcggtctgaaggatgcactccgggatagggcagtcccggctcttgctaagtccttcagcaagtcgaaagacacgatgtgagcctatttt

SEQ ID NO: 536 - DNA, 130 bp, synthetic construct
  source 1..130: mol_type = other DNA
gtctcaatcaaccgatagaaatatcggtccggttgagaagagcatcggctgaaggatgcactccgggatagggcagtcccggctcttgctaagtccttcagcaagcgaaagcacgatgtgagcctatttt

SEQ ID NO: 537 - DNA, 126 bp, synthetic construct
  source 1..126: mol_type = other DNA
gtctcaatcaaccgagaaatcggtccggttgaaaagagcatcggctgaaggatgcactccgggatagggcagtcccggctcttgctaagtccttcagcaagcgaaagcacgatgtgagcctatttt

SEQ ID NO: 538 - DNA, 153 bp, synthetic construct
  source 1..153: mol_type = other DNA
ctccaatgactagggtgggcgtttcaatcaaccgatagaaatatcggtccggttgaaaagagcatcggtctgaaggatgcactccgggatagggcagtcccggctcttgctaagtccttcagcaagtcgaaagacacgatgtgagcctatttt

SEQ ID NO: 539 - DNA, 153 bp, synthetic construct
  source 1..153: mol_type = other DNA
atgggctggctgaggcctgggtttcaatcaaccgatagaaatatcggtccggttgaaaagagcatcggtctgaaggatgcactccgggatagggcagtcccggctcttgctaagtccttcagcaagtcgaaagacacgatgtgagcctatttt

SEQ ID NO: 540 - DNA, 153 bp, synthetic construct
  source 1..153: mol_type = other DNA
agacaaggcacttggttgatgtttcaatcaaccgatagaaatatcggtccggttgaaaagagcatcggtctgaaggatgcactccgggatagggcagtcccggctcttgctaagtccttcagcaagtcgaaagacacgatgtgagcctatttt

SEQ ID NO: 541 - DNA, 153 bp, synthetic construct
  source 1..153: mol_type = other DNA
cccacacgcacacactcactgtttcaatcaaccgatagaaatatcggtccggttgaaaagagcatcggtctgaaggatgcactccgggatagggcagtcccggctcttgctaagtccttcagcaagtcgaaagacacgatgtgagcctatttt

SEQ ID NO: 542 - DNA, 153 bp, synthetic construct
  source 1..153: mol_type = other DNA
gacttgggtgagcgatggacgtttcaatcaaccgatagaaatatcggtccggttgaaaagagcatcggtctgaaggatgcactccgggatagggcagtcccggctcttgctaagtccttcagcaagtcgaaagacacgatgtgagcctatttt

SEQ ID NO: 543 - DNA, 153 bp, synthetic construct
  source 1..153: mol_type = other DNA
ggacctccaggtgcagaagtgtttcaatcaaccgatagaaatatcggtccggttgaaaagagcatcggtctgaaggatgcactccgggatagggcagtcccggctcttgctaagtccttcagcaagtcgaaagacacgatgtgagcctatttt

SEQ ID NO: 544 - DNA, 153 bp, synthetic construct
  source 1..153: mol_type = other DNA
ctgtgaagagccagtggccagtttcaatcaaccgatagaaatatcggtccggttgaaaagagcatcggtctgaaggatgcactccgggatagggcagtcccggctcttgctaagtccttcagcaagtcgaaagacacgatgtgagcctatttt

SEQ ID NO: 545 - DNA, 153 bp, synthetic construct
  source 1..153: mol_type = other DNA
ggacaaaggtcctcaaagacgtttcaatcaaccgatagaaatatcggtccggttgaaaagagcatcggtctgaaggatgcactccgggatagggcagtcccggctcttgctaagtccttcagcaagtcgaaagacacgatgtgagcctatttt

SEQ ID NO: 546 - DNA, 153 bp, synthetic construct
  source 1..153: mol_type = other DNA
gacttgggtgagcgatggacgcttcaatcaaccgatagaaatatcggtccggttgaagagagcatcggtctgaaggatgcactccgggatagggcagtcccggctcttgctaagtccttcagcaagtcgaaagacacgatgtgagcctatttt

SEQ ID NO: 547 - DNA, 153 bp, synthetic construct
  source 1..153: mol_type = other DNA
gacttgggtgagcgatggacgtctcaatcaaccgatagaaatatcggtccggttgagaagagcatcggtctgaaggatgcactccgggatagggcagtcccggctcttgctaagtccttcagcaagtcgaaagacacgatgtgagcctatttt

SEQ ID NO: 548 - DNA, 153 bp, synthetic construct
  source 1..153: mol_type = other DNA
gacttgggtgagcgatggacgtttcgatcaaccgatagaaatatcggtccggtcgaaaagagcatcggtctgaaggatgcactccgggatagggcagtcccggctcttgctaagtccttcagcaagtcgaaagacacgatgtgagcctatttt

SEQ ID NO: 549 - DNA, 153 bp, synthetic construct
  source 1..153: mol_type = other DNA
gacttgggtgagcgatggacgtttcaatcagccgatagaaatatcggcccggttgaaaagagcatcggtctgaaggatgcactccgggatagggcagtcccggctcttgctaagtccttcagcaagtcgaaagacacgatgtgagcctatttt

SEQ ID NO: 550 - DNA, 153 bp, synthetic construct
  source 1..153: mol_type = other DNA
gacttgggtgagcgatggacgtttcaatcacccgatagaaatatcgggccggttgaaaagagcatcggtctgaaggatgcactccgggatagggcagtcccggctcttgctaagtccttcagcaagtcgaaagacacgatgtgagcctatttt

SEQ ID NO: 551 - DNA, 151 bp, synthetic construct
  source 1..151: mol_type = other DNA
gacttgggtgagcgatggacgtttcaatcaaccgatgaaaatcggtccggttgaaaagagcatcggtctgaaggatgcactccgggatagggcagtcccggctcttgctaagtccttcagcaagtcgaaagacacgatgtgagcctatttt

SEQ ID NO: 552 - DNA, 149 bp, synthetic construct
  source 1..149: mol_type = other DNA
gacttgggtgagcgatggacgtttcaatcaaccgagaaatcggtccggttgaaaagagcatcggtctgaaggatgcactccgggatagggcagtcccggctcttgctaagtccttcagcaagtcgaaagacacgatgtgagcctatttt

SEQ ID NO: 553 - DNA, 152 bp, synthetic construct
  source 1..152: mol_type = other DNA
gacttgggtgagcgatggacgtttcaatcaaccgatagaaatatcggtccggttgaaaagagcatcggctgaaggatgcactccgggatagggcagtcccggctcttgctaagtccttcagcaagtcgaaagacacgatgtgagcctatttt

SEQ ID NO: 554 - DNA, 151 bp, synthetic construct
  source 1..151: mol_type = other DNA
gacttgggtgagcgatggacgtttcaatcaaccgatagaaatatcggtccggttgaaaagagcatcggtctgaaggatgcactccgggatagggcagtcccggctcttgctaagtccttcagcaagcgaaagcacgatgtgagcctatttt

SEQ ID NO: 555 - DNA, 153 bp, synthetic construct
  source 1..153: mol_type = other DNA
ggacctccaggtgcagaagtgcttcaatcaaccgatagaaatatcggtccggttgaagagagcatcggtctgaaggatgcactccgggatagggcagtcccggctcttgctaagtccttcagcaagtcgaaagacacgatgtgagcctatttt

SEQ ID NO: 556 - DNA, 153 bp, synthetic construct
  source 1..153: mol_type = other DNA
ggacctccaggtgcagaagtgtctcaatcaaccgatagaaatatcggtccggttgagaagagcatcggtctgaaggatgcactccgggatagggcagtcccggctcttgctaagtccttcagcaagtcgaaagacacgatgtgagcctatttt

SEQ ID NO: 557 - DNA, 153 bp, synthetic construct
  source 1..153: mol_type = other DNA
ggacctccaggtgcagaagtgtttcgatcaaccgatagaaatatcggtccggtcgaaaagagcatcggtctgaaggatgcactccgggatagggcagtcccggctcttgctaagtccttcagcaagtcgaaagacacgatgtgagcctatttt

SEQ ID NO: 558 - DNA, 153 bp, synthetic construct
  source 1..153: mol_type = other DNA
ggacctccaggtgcagaagtgtttcaatcagccgatagaaatatcggcccggttgaaaagagcatcggtctgaaggatgcactccgggatagggcagtcccggctcttgctaagtccttcagcaagtcgaaagacacgatgtgagcctatttt

SEQ ID NO: 559 - DNA, 153 bp, synthetic construct
  source 1..153: mol_type = other DNA
ggacctccaggtgcagaagtgtttcaatcacccgatagaaatatcgggccggttgaaaagagcatcggtctgaaggatgcactccgggatagggcagtcccggctcttgctaagtccttcagcaagtcgaaagacacgatgtgagcctatttt

SEQ ID NO: 560 - DNA, 151 bp, synthetic construct
  source 1..151: mol_type = other DNA
ggacctccaggtgcagaagtgtttcaatcaaccgatgaaaatcggtccggttgaaaagagcatcggtctgaaggatgcactccgggatagggcagtcccggctcttgctaagtccttcagcaagtcgaaagacacgatgtgagcctatttt

SEQ ID NO: 561 - DNA, 149 bp, synthetic construct
  source 1..149: mol_type = other DNA
ggacctccaggtgcagaagtgtttcaatcaaccgagaaatcggtccggttgaaaagagcatcggtctgaaggatgcactccgggatagggcagtcccggctcttgctaagtccttcagcaagtcgaaagacacgatgtgagcctatttt

SEQ ID NO: 562 - DNA, 152 bp, synthetic construct
  source 1..152: mol_type = other DNA
ggacctccaggtgcagaagtgtttcaatcaaccgatagaaatatcggtccggttgaaaagagcatcggctgaaggatgcactccgggatagggcagtcccggctcttgctaagtccttcagcaagtcgaaagacacgatgtgagcctatttt

SEQ ID NO: 563 - DNA, 151 bp, synthetic construct
  source 1..151: mol_type = other DNA
ggacctccaggtgcagaagtgtttcaatcaaccgatagaaatatcggtccggttgaaaagagcatcggtctgaaggatgcactccgggatagggcagtcccggctcttgctaagtccttcagcaagcgaaagcacgatgtgagcctatttt

SEQ ID NO: 564 - DNA, 153 bp, synthetic construct
  source 1..153: mol_type = other DNA
ctgtgaagagccagtggccagcttcaatcaaccgatagaaatatcggtccggttgaagagagcatcggtctgaaggatgcactccgggatagggcagtcccggctcttgctaagtccttcagcaagtcgaaagacacgatgtgagcctatttt

SEQ ID NO: 565 - DNA, 153 bp, synthetic construct
  source 1..153: mol_type = other DNA
ctgtgaagagccagtggccagtctcaatcaaccgatagaaatatcggtccggttgagaagagcatcggtctgaaggatgcactccgggatagggcagtcccggctcttgctaagtccttcagcaagtcgaaagacacgatgtgagcctatttt

SEQ ID NO: 566 - DNA, 153 bp, synthetic construct
  source 1..153: mol_type = other DNA
ctgtgaagagccagtggccagtttcgatcaaccgatagaaatatcggtccggtcgaaaagagcatcggtctgaaggatgcactccgggatagggcagtcccggctcttgctaagtccttcagcaagtcgaaagacacgatgtgagcctatttt

SEQ ID NO: 567 - DNA, 153 bp, synthetic construct
  source 1..153: mol_type = other DNA
ctgtgaagagccagtggccagtttcaatcagccgatagaaatatcggcccggttgaaaagagcatcggtctgaaggatgcactccgggatagggcagtcccggctcttgctaagtccttcagcaagtcgaaagacacgatgtgagcctatttt

SEQ ID NO: 568 - DNA, 153 bp, synthetic construct
  source 1..153: mol_type = other DNA
ctgtgaagagccagtggccagtttcaatcacccgatagaaatatcgggccggttgaaaagagcatcggtctgaaggatgcactccgggatagggcagtcccggctcttgctaagtccttcagcaagtcgaaagacacgatgtgagcctatttt

SEQ ID NO: 569 - DNA, 151 bp, synthetic construct
  source 1..151: mol_type = other DNA
ctgtgaagagccagtggccagtttcaatcaaccgatgaaaatcggtccggttgaaaagagcatcggtctgaaggatgcactccgggatagggcagtcccggctcttgctaagtccttcagcaagtcgaaagacacgatgtgagcctatttt

SEQ ID NO: 570 - DNA, 149 bp, synthetic construct
  source 1..149: mol_type = other DNA
ctgtgaagagccagtggccagtttcaatcaaccgagaaatcggtccggttgaaaagagcatcggtctgaaggatgcactccgggatagggcagtcccggctcttgctaagtccttcagcaagtcgaaagacacgatgtgagcctatttt

SEQ ID NO: 571 - DNA, 152 bp, synthetic construct
  source 1..152: mol_type = other DNA
ctgtgaagagccagtggccagtttcaatcaaccgatagaaatatcggtccggttgaaaagagcatcggctgaaggatgcactccgggatagggcagtcccggctcttgctaagtccttcagcaagtcgaaagacacgatgtgagcctatttt

SEQ ID NO: 572 - DNA, 151 bp, synthetic construct
  source 1..151: mol_type = other DNA
ctgtgaagagccagtggccagtttcaatcaaccgatagaaatatcggtccggttgaaaagagcatcggtctgaaggatgcactccgggatagggcagtcccggctcttgctaagtccttcagcaagcgaaagcacgatgtgagcctatttt

SEQ ID NO: 573 - DNA, 153 bp, synthetic construct
  source 1..153: mol_type = other DNA
gacttgggtgagcgatggacgcctcaatcaaccgatagaaatatcggtccggttgaggagagcatcggtctgaaggatgcactccgggatagggcagtcccggctcttgctaagtccttcagcaagtcgaaagacacgatgtgagcctatttt

SEQ ID NO: 574 - DNA, 153 bp, synthetic construct
  source 1..153: mol_type = other DNA
gacttgggtgagcgatggacgcctcgatcaaccgatagaaatatcggtccggtcgaggagagcatcggtctgaaggatgcactccgggatagggcagtcccggctcttgctaagtccttcagcaagtcgaaagacacgatgtgagcctatttt

SEQ ID NO: 575 - DNA, 150 bp, synthetic construct
  source 1..150: mol_type = other DNA
gacttgggtgagcgatggacgtctcaatcaaccgatagaaatatcggtccggttgagaagagcatcggctgaaggatgcactccgggatagggcagtcccggctcttgctaagtccttcagcaagcgaaagcacgatgtgagcctatttt

SEQ ID NO: 576 - DNA, 146 bp, synthetic construct
  source 1..146: mol_type = other DNA
gacttgggtgagcgatggacgtctcaatcaaccgagaaatcggtccggttgaaaagagcatcggctgaaggatgcactccgggatagggcagtcccggctcttgctaagtccttcagcaagcgaaagcacgatgtgagcctatttt

SEQ ID NO: 577 - DNA, 153 bp, synthetic construct
  source 1..153: mol_type = other DNA
ggacctccaggtgcagaagtgcctcaatcaaccgatagaaatatcggtccggttgaggagagcatcggtctgaaggatgcactccgggatagggcagtcccggctcttgctaagtccttcagcaagtcgaaagacacgatgtgagcctatttt

SEQ ID NO: 578 - DNA, 153 bp, synthetic construct
  source 1..153: mol_type = other DNA
ggacctccaggtgcagaagtgcctcgatcaaccgatagaaatatcggtccggtcgaggagagcatcggtctgaaggatgcactccgggatagggcagtcccggctcttgctaagtccttcagcaagtcgaaagacacgatgtgagcctatttt

SEQ ID NO: 579 - DNA, 150 bp, synthetic construct
  source 1..150: mol_type = other DNA
ggacctccaggtgcagaagtgtctcaatcaaccgatagaaatatcggtccggttgagaagagcatcggctgaaggatgcactccgggatagggcagtcccggctcttgctaagtccttcagcaagcgaaagcacgatgtgagcctatttt

SEQ ID NO: 580 - DNA, 146 bp, synthetic construct
  source 1..146: mol_type = other DNA
ggacctccaggtgcagaagtgtctcaatcaaccgagaaatcggtccggttgaaaagagcatcggctgaaggatgcactccgggatagggcagtcccggctcttgctaagtccttcagcaagcgaaagcacgatgtgagcctatttt

SEQ ID NO: 581 - DNA, 153 bp, synthetic construct
  source 1..153: mol_type = other DNA
ctgtgaagagccagtggccagcctcaatcaaccgatagaaatatcggtccggttgaggagagcatcggtctgaaggatgcactccgggatagggcagtcccggctcttgctaagtccttcagcaagtcgaaagacacgatgtgagcctatttt

SEQ ID NO: 582 - DNA, 153 bp, synthetic construct
  source 1..153: mol_type = other DNA
ctgtgaagagccagtggccagcctcgatcaaccgatagaaatatcggtccggtcgaggagagcatcggtctgaaggatgcactccgggatagggcagtcccggctcttgctaagtccttcagcaagtcgaaagacacgatgtgagcctatttt

SEQ ID NO: 583 - DNA, 150 bp, synthetic construct
  source 1..150: mol_type = other DNA
ctgtgaagagccagtggccagtctcaatcaaccgatagaaatatcggtccggttgagaagagcatcggctgaaggatgcactccgggatagggcagtcccggctcttgctaagtccttcagcaagcgaaagcacgatgtgagcctatttt

SEQ ID NO: 584 - DNA, 146 bp, synthetic construct
  source 1..146: mol_type = other DNA
ctgtgaagagccagtggccagtctcaatcaaccgagaaatcggtccggttgaaaagagcatcggctgaaggatgcactccgggatagggcagtcccggctcttgctaagtccttcagcaagcgaaagcacgatgtgagcctatttt

SEQ ID NO: 585 - DNA, 153 bp, synthetic construct
  source 1..153: mol_type = other DNA
agccacgtctacagcagggcgtttcaatcaaccgatagaaatatcggtccggttgaaaagagcatcggtctgaaggatgcactccgggatagggcagtcccggctcttgctaagtccttcagcaagtcgaaagacacgatgtgagcctatttt

SEQ ID NO: 586 - DNA, 153 bp, synthetic construct
  source 1..153: mol_type = other DNA
acagcagggctgcctcggacgtttcaatcaaccgatagaaatatcggtccggttgaaaagagcatcggtctgaaggatgcactccgggatagggcagtcccggctcttgctaagtccttcagcaagtcgaaagacacgatgtgagcctatttt

SEQ ID NO: 587 - DNA, 153 bp, synthetic construct
  source 1..153: mol_type = other DNA
gagggatcctgggagcccttgtttcaatcaaccgatagaaatatcggtccggttgaaaagagcatcggtctgaaggatgcactccgggatagggcagtcccggctcttgctaagtccttcagcaagtcgaaagacacgatgtgagcctatttt

SEQ ID NO: 588 - DNA, 153 bp, synthetic construct
  source 1..153: mol_type = other DNA
agccacgtctacagcagggcgtctcaatcaaccgatagaaatatcggtccggttgagaagagcatcggtctgaaggatgcactccgggatagggcagtcccggctcttgctaagtccttcagcaagtcgaaagacacgatgtgagcctatttt

SEQ ID NO: 589 - DNA, 153 bp, synthetic construct
  source 1..153: mol_type = other DNA
acagcagggctgcctcggacgtctcaatcaaccgatagaaatatcggtccggttgagaagagcatcggtctgaaggatgcactccgggatagggcagtcccggctcttgctaagtccttcagcaagtcgaaagacacgatgtgagcctatttt

SEQ ID NO: 590 - DNA, 153 bp, synthetic construct
  source 1..153: mol_type = other DNA
gagggatcctgggagcccttgtctcaatcaaccgatagaaatatcggtccggttgagaagagcatcggtctgaaggatgcactccgggatagggcagtcccggctcttgctaagtccttcagcaagtcgaaagacacgatgtgagcctatttt

SEQ ID NO: 591 - DNA, 157 bp, synthetic construct
  source 1..157: mol_type = other DNA
ttacagccacgtctacagcagggcgtttcaatcaaccgatagaaatatcggtccggttgaaaagagcatcggtctgaaggatgcactccgggatagggcagtcccggctcttgctaagtccttcagcaagtcgaaagacacgatgtgagcctatttt

SEQ ID NO: 592 - DNA, 157 bp, synthetic construct
  source 1..157: mol_type = other DNA
gtctacagcagggctgcctcggacgtttcaatcaaccgatagaaatatcggtccggttgaaaagagcatcggtctgaaggatgcactccgggatagggcagtcccggctcttgctaagtccttcagcaagtcgaaagacacgatgtgagcctatttt

SEQ ID NO: 593 - DNA, 157 bp, synthetic construct
  source 1..157: mol_type = other DNA
ctctgagggatcctgggagcccttgtttcaatcaaccgatagaaatatcggtccggttgaaaagagcatcggtctgaaggatgcactccgggatagggcagtcccggctcttgctaagtccttcagcaagtcgaaagacacgatgtgagcctatttt

SEQ ID NO: 594 - DNA, 157 bp, synthetic construct
  source 1..157: mol_type = other DNA
ttacagccacgtctacagcagggcgtctcaatcaaccgatagaaatatcggtccggttgagaagagcatcggtctgaaggatgcactccgggatagggcagtcccggctcttgctaagtccttcagcaagtcgaaagacacgatgtgagcctatttt

SEQ ID NO: 595 - DNA, 157 bp, synthetic construct
  source 1..157: mol_type = other DNA
gtctacagcagggctgcctcggacgtctcaatcaaccgatagaaatatcggtccggttgagaagagcatcggtctgaaggatgcactccgggatagggcagtcccggctcttgctaagtccttcagcaagtcgaaagacacgatgtgagcctatttt

SEQ ID NO: 596 - DNA, 157 bp, synthetic construct
  source 1..157: mol_type = other DNA
ctctgagggatcctgggagcccttgtctcaatcaaccgatagaaatatcggtccggttgagaagagcatcggtctgaaggatgcactccgggatagggcagtcccggctcttgctaagtccttcagcaagtcgaaagacacgatgtgagcctatttt

SEQ ID NO: 597 - DNA, 153 bp, synthetic construct
  source 1..153: mol_type = other DNA
ctccctatcagtgatagagagtttcaatcaaccgatagaaatatcggtccggttgaaaagagcatcggtctgaaggatgcactccgggatagggcagtcccggctcttgctaagtccttcagcaagtcgaaagacacgatgtgagcctatttt

SEQ ID NO: 598 - DNA, 153 bp, synthetic construct
  source 1..153: mol_type = other DNA
ctccctatcagtgatagagagtctcaatcaaccgatagaaatatcggtccggttgagaagagcatcggtctgaaggatgcactccgggatagggcagtcccggctcttgctaagtccttcagcaagtcgaaagacacgatgtgagcctatttt

SEQ ID NO: 599 - DNA, 150 bp, synthetic construct
  source 1..150: mol_type = other DNA
agccacgtctacagcagggcgtctcaatcaaccgatagaaatatcggtccggttgagaagagcatcggctgaaggatgcactccgggatagggcagtcccggctcttgctaagtccttcagcaagcgaaagcacgatgtgagcctatttt

SEQ ID NO: 600 - DNA, 150 bp, synthetic construct
  source 1..150: mol_type = other DNA
acagcagggctgcctcggacgtctcaatcaaccgatagaaatatcggtccggttgagaagagcatcggctgaaggatgcactccgggatagggcagtcccggctcttgctaagtccttcagcaagcgaaagcacgatgtgagcctatttt

SEQ ID NO: 601 - DNA, 150 bp, synthetic construct
  source 1..150: mol_type = other DNA
gagggatcctgggagcccttgtctcaatcaaccgatagaaatatcggtccggttgagaagagcatcggctgaaggatgcactccgggatagggcagtcccggctcttgctaagtccttcagcaagcgaaagcacgatgtgagcctatttt

SEQ ID NO: 602 - DNA, 150 bp, synthetic construct; identical to SEQ ID NO: 599
  source 1..150: mol_type = other DNA

SEQ ID NO: 603 - DNA, 150 bp, synthetic construct; identical to SEQ ID NO: 600
  source 1..150: mol_type = other DNA

SEQ ID NO: 604 - DNA, 150 bp, synthetic construct; identical to SEQ ID NO: 601
  source 1..150: mol_type = other DNA

SEQ ID NO: 605 - DNA, 153 bp, synthetic construct; identical to SEQ ID NO: 588
  source 1..153: mol_type = other DNA

SEQ ID NO: 606 - DNA, 153 bp, synthetic construct; identical to SEQ ID NO: 589
  source 1..153: mol_type = other DNA

SEQ ID NO: 607 - DNA, 153 bp, synthetic construct; identical to SEQ ID NO: 590
  source 1..153: mol_type = other DNA

SEQ ID NO: 608 - DNA, 157 bp, synthetic construct; identical to SEQ ID NO: 594
  source 1..157: mol_type = other DNA

SEQ ID NO: 609 - DNA, 157 bp, synthetic construct; identical to SEQ ID NO: 595
  source 1..157: mol_type = other DNA

SEQ ID NO: 610 - DNA, 157 bp, synthetic construct; identical to SEQ ID NO: 596
  source 1..157: mol_type = other DNA

SEQ ID NO: 611 - RNA, 153 nt, synthetic construct
  source 1..153: mol_type = other RNA
ctccaatgactagggtgggcgtttcaatcaaccgatagaaatatcggtccggttgaaaagagcatcggtctgaaggatgcactccgggatagggcagtcccggctcttgctaagtccttcagcaagtcgaaagacacgatgtgagcctatttt

SEQ ID NO: 612 - RNA, 153 nt, synthetic construct
  source 1..153: mol_type = other RNA
atgggctggctgaggcctgggtttcaatcaaccgatagaaatatcggtccggttgaaaagagcatcggtctgaaggatgcactccgggatagggcagtcccggctcttgctaagtccttcagcaagtcgaaagacacgatgtgagcctatttt

SEQ ID NO: 613 - RNA, 153 nt, synthetic construct
  source 1..153: mol_type = other RNA
agacaaggcacttggttgatgtttcaatcaaccgatagaaatatcggtccggttgaaaagagcatcggtctgaaggatgcactccgggatagggcagtcccggctcttgctaagtccttcagcaagtcgaaagacacgatgtgagcctatttt

SEQ ID NO: 614 - RNA, 153 nt, synthetic construct
  source 1..153: mol_type = other RNA
cccacacgcacacactcactgtttcaatcaaccgatagaaatatcggtccggttgaaaagagcatcggtctgaaggatgcactccgggatagggcagtcccggctcttgctaagtccttcagcaagtcgaaagacacgatgtgagcctatttt

SEQ ID NO: 615 - RNA, 153 nt, synthetic construct
  source 1..153: mol_type = other RNA
gacttgggtgagcgatggacgtttcaatcaaccgatagaaatatcggtccggttgaaaagagcatcggtctgaaggatgcactccgggatagggcagtcccggctcttgctaagtccttcagcaagtcgaaagacacgatgtgagcctatttt

SEQ ID NO: 616 - RNA, 153 nt, synthetic construct
  source 1..153: mol_type = other RNA
ggacctccaggtgcagaagtgtttcaatcaaccgatagaaatatcggtccggttgaaaagagcatcggtctgaaggatgcactccgggatagggcagtcccggctcttgctaagtccttcagcaagtcgaaagacacgatgtgagcctatttt

SEQ ID NO: 617 - RNA, 153 nt, synthetic construct
  source 1..153: mol_type = other RNA
ctgtgaagagccagtggccagtttcaatcaaccgatagaaatatcggtccggttgaaaagagcatcggtctgaaggatgcactccgggatagggcagtcccggctcttgctaagtccttcagcaagtcgaaagacacgatgtgagcctatttt

SEQ ID NO: 618 - RNA, 153 nt, synthetic construct
  source 1..153: mol_type = other RNA
ggacaaaggtcctcaaagacgtttcaatcaaccgatagaaatatcggtccggttgaaaagagcatcggtctgaaggatgcactccgggatagggcagtcccggctcttgctaagtccttcagcaagtcgaaagacacgatgtgagcctatttt

SEQ ID NO: 619 - RNA, 153 nt, synthetic construct
  source 1..153: mol_type = other RNA
gacttgggtgagcgatggacgcttcaatcaaccgatagaaatatcggtccggttgaagagagcatcggtctgaaggatgcactccgggatagggcagtcccggctcttgctaagtccttcagcaagtcgaaagacacgatgtgagcctatttt

SEQ ID NO: 620 - RNA, 153 nt, synthetic construct
  source 1..153: mol_type = other RNA
gacttgggtgagcgatggacgtctcaatcaaccgatagaaatatcggtccggttgagaagagcatcggtctgaaggatgcactccgggatagggcagtcccggctcttgctaagtccttcagcaagtcgaaagacacgatgtgagcctatttt

SEQ ID NO: 621 - RNA, 153 nt, synthetic construct
  source 1..153: mol_type = other RNA
gacttgggtgagcgatggacgtttcgatcaaccgatagaaatatcggtccggtcgaaaagagcatcggtctgaaggatgcactccgggatagggcagtcccggctcttgctaagtccttcagcaagtcgaaagacacgatgtgagcctatttt

SEQ ID NO: 622 - RNA, 153 nt, synthetic construct
  source 1..153: mol_type = other RNA
gacttgggtgagcgatggacgtttcaatcagccgatagaaatatcggcccggttgaaaagagcatcggtctgaaggatgcactccgggatagggcagtcccggctcttgctaagtccttcagcaagtcgaaagacacgatgtgagcctatttt

SEQ ID NO: 623 - RNA, 153 nt, synthetic construct
  source 1..153: mol_type = other RNA
gacttgggtgagcgatggacgtttcaatcacccgatagaaatatcgggccggttgaaaagagcatcggtctgaaggatgcactccgggatagggcagtcccggctcttgctaagtccttcagcaagtcgaaagacacgatgtgagcctatttt

SEQ ID NO: 624 - RNA, 151 nt, synthetic construct
  source 1..151: mol_type = other RNA
gacttgggtgagcgatggacgtttcaatcaaccgatgaaaatcggtccggttgaaaagagcatcggtctgaaggatgcactccgggatagggcagtcccggctcttgctaagtccttcagcaagtcgaaagacacgatgtgagcctatttt

SEQ ID NO: 625 - RNA, 149 nt, synthetic construct
  source 1..149: mol_type = other RNA
gacttgggtgagcgatggacgtttcaatcaaccgagaaatcggtccggttgaaaagagcatcggtctgaaggatgcactccgggatagggcagtcccggctcttgctaagtccttcagcaagtcgaaagacacgatgtgagcctatttt

SEQ ID NO: 626 - RNA, 152 nt, synthetic construct
  source 1..152: mol_type = other RNA
gacttgggtgagcgatggacgtttcaatcaaccgatagaaatatcggtccggttgaaaagagcatcggctgaaggatgcactccgggatagggcagtcccggctcttgctaagtccttcagcaagtcgaaagacacgatgtgagcctatttt

SEQ ID NO: 627 - RNA, 151 nt, synthetic construct
  source 1..151: mol_type = other RNA
gacttgggtgagcgatggacgtttcaatcaaccgatagaaatatcggtccggttgaaaagagcatcggtctgaaggatgcactccgggatagggcagtcccggctcttgctaagtccttcagcaagcgaaagcacgatgtgagcctatttt

SEQ ID NO: 628 - RNA, 153 nt, synthetic construct
  source 1..153: mol_type = other RNA
ggacctccaggtgcagaagtgcttcaatcaaccgatagaaatatcggtccggttgaagagagcatcggtctgaaggatgcactccgggatagggcagtcccggctcttgctaagtccttcagcaagtcgaaagacacgatgtgagcctatttt

SEQ ID NO: 629 - RNA, 153 nt, synthetic construct
  source 1..153: mol_type = other RNA
ggacctccaggtgcagaagtgtctcaatcaaccgatagaaatatcggtccggttgagaagagcatcggtctgaaggatgcactccgggatagggcagtcccggctcttgctaagtccttcagcaagtcgaaagacacgatgtgagcctatttt

SEQ ID NO: 630 - RNA, 153 nt, synthetic construct
  source 1..153: mol_type = other RNA
ggacctccaggtgcagaagtgtttcgatcaaccgatagaaatatcggtccggtcgaaaagagcatcggtctgaaggatgcactccgggatagggcagtcccggctcttgctaagtccttcagcaagtcgaaagacacgatgtgagcctatttt

SEQ ID NO: 631 - RNA, 153 nt, synthetic construct
  source 1..153: mol_type = other RNA
ggacctccaggtgcagaagtgtttcaatcagccgatagaaatatcggcccggttgaaaagagcatcggtctgaaggatgcactccgggatagggcagtcccggctcttgctaagtccttcagcaagtcgaaagacacgatgtgagcctatttt

SEQ ID NO: 632 - RNA, 153 nt, synthetic construct
  source 1..153: mol_type = other RNA
ggacctccaggtgcagaagtgtttcaatcacccgatagaaatatcgggccggttgaaaagagcatcggtctgaaggatgcactccgggatagggcagtcccggctcttgctaagtccttcagcaagtcgaaagacacgatgtgagcctatttt

SEQ ID NO: 633 - RNA, 151 nt, synthetic construct
  source 1..151: mol_type = other RNA
ggacctccaggtgcagaagtgtttcaatcaaccgatgaaaatcggtccggttgaaaagagcatcggtctgaaggatgcactccgggatagggcagtcccggctcttgctaagtccttcagcaagtcgaaagacacgatgtgagcctatttt

SEQ ID NO: 634 - RNA, 149 nt, synthetic construct
  source 1..149: mol_type = other RNA
ggacctccaggtgcagaagtgtttcaatcaaccgagaaatcggtccggttgaaaagagcatcggtctgaaggatgcactccgggatagggcagtcccggctcttgctaagtccttcagcaagtcgaaagacacgatgtgagcctatttt

SEQ ID NO: 635 - RNA, 152 nt, synthetic construct
  source 1..152: mol_type = other RNA
ggacctccaggtgcagaagtgtttcaatcaaccgatagaaatatcggtccggttgaaaagagcatcggctgaaggatgcactccgggatagggcagtcccggctcttgctaagtccttcagcaagtcgaaagacacgatgtgagcctatttt

SEQ ID NO: 636 - RNA, 151 nt, synthetic construct
  source 1..151: mol_type = other RNA
ggacctccaggtgcagaagtgtttcaatcaaccgatagaaatatcggtccggttgaaaagagcatcggtctgaaggatgcactccgggatagggcagtcccggctcttgctaagtccttcagcaagcgaaagcacgatgtgagcctatttt

SEQ ID NO: 637 - RNA, 153 nt, synthetic construct
  source 1..153: mol_type = other RNA
ctgtgaagagccagtggccagcttcaatcaaccgatagaaatatcggtccggttgaagagagcatcggtctgaaggatgcactccgggatagggcagtcccggctcttgctaagtccttcagcaagtcgaaagacacgatgtgagcctatttt

SEQ ID NO: 638 - RNA, 153 nt, synthetic construct
  source 1..153: mol_type = other RNA
ctgtgaagagccagtggccagtctcaatcaaccgatagaaatatcggtccggttgagaagagcatcggtctgaaggatgcactccgggatagggcagtcccggctcttgctaagtccttcagcaagtcgaaagacacgatgtgagcctatttt

SEQ ID NO: 639 - RNA, 153 nt, synthetic construct
  source 1..153: mol_type = other RNA
ctgtgaagagccagtggccagtttcgatcaaccgatagaaatatcggtccggtcgaaaagagcatcggtctgaaggatgcactccgggatagggcagtcccggctcttgctaagtccttcagcaagtcgaaagacacgatgtgagcctatttt

SEQ ID NO: 640 - RNA, 153 nt, synthetic construct
  source 1..153: mol_type = other RNA
ctgtgaagagccagtggccagtttcaatcagccgatagaaatatcggcccggttgaaaagagcatcggtctgaaggatgcactccgggatagggcagtcccggctcttgctaagtccttcagcaagtcgaaagacacgatgtgagcctatttt

SEQ ID NO: 641 - RNA, 153 nt, synthetic construct
  source 1..153: mol_type = other RNA
ctgtgaagagccagtggccagtttcaatcacccgatagaaatatcgggccggttgaaaagagcatcggtctgaaggatgcactccgggatagggcagtcccggctcttgctaagtccttcagcaagtcgaaagacacgatgtgagcctatttt

SEQ ID NO: 642 - RNA, 151 nt, synthetic construct
  source 1..151: mol_type = other RNA
ctgtgaagagccagtggccagtttcaatcaaccgatgaaaatcggtccggttgaaaagagcatcggtctgaaggatgcactccgggatagggcagtcccggctcttgctaagtccttcagcaagtcgaaagacacgatgtgagcctatttt

SEQ ID NO: 643 - RNA, 149 nt, synthetic construct
  source 1..149: mol_type = other RNA
ctgtgaagagccagtggccagtttcaatcaaccgagaaatcggtccggttgaaaagagcatcggtctgaaggatgcactccgggatagggcagtcccggctcttgctaagtccttcagcaagtcgaaagacacgatgtgagcctatttt

SEQ ID NO: 644 - RNA, 152 nt, synthetic construct
  source 1..152: mol_type = other RNA
ctgtgaagagccagtggccagtttcaatcaaccgatagaaatatcggtccggttgaaaagagcatcggctgaaggatgcactccgggatagggcagtcccggctcttgctaagtccttcagcaagtcgaaagacacgatgtgagcctatttt

SEQ ID NO: 645 - RNA, 151 nt, synthetic construct
  source 1..151: mol_type = other RNA
ctgtgaagagccagtggccagtttcaatcaaccgatagaaatatcggtccggttgaaaagagcatcggtctgaaggatgcactccgggatagggcagtcccggctcttgctaagtccttcagcaagcgaaagcacgatgtgagcctatttt

SEQ ID NO: 646 - RNA, 153 nt, synthetic construct
  source 1..153: mol_type = other RNA
gacttgggtgagcgatggacgcctcaatcaaccgatagaaatatcggtccggttgaggagagcatcggtctgaaggatgcactccgggatagggcagtcccggctcttgctaagtccttcagcaagtcgaaagacacgatgtgagcctatttt

SEQ ID NO: 647 - RNA, 153 nt, synthetic construct
  source 1..153: mol_type = other RNA
gacttgggtgagcgatggacgcctcgatcaaccgatagaaatatcggtccggtcgaggagagcatcggtctgaaggatgcactccgggatagggcagtcccggctcttgctaagtccttcagcaagtcgaaagacacgatgtgagcctatttt

SEQ ID NO: 648 - RNA, 150 nt, synthetic construct
  source 1..150: mol_type = other RNA
gacttgggtgagcgatggacgtctcaatcaaccgatagaaatatcggtccggttgagaagagcatcggctgaaggatgcactccgggatagggcagtcccggctcttgctaagtccttcagcaagcgaaagcacgatgtgagcctatttt

SEQ ID NO: 649 - RNA, 146 nt, synthetic construct
  source 1..146: mol_type = other RNA
gacttgggtgagcgatggacgtctcaatcaaccgagaaatcggtccggttgaaaagagcatcggctgaaggatgcactccgggatagggcagtcccggctcttgctaagtccttcagcaagcgaaagcacgatgtgagcctatttt

SEQ ID NO: 650 - RNA, 153 nt, synthetic construct
  source 1..153: mol_type = other RNA
ggacctccaggtgcagaagtgcctcaatcaaccgatagaaatatcggtccggttgaggagagcatcggtctgaaggatgcactccgggatagggcagtcccggctcttgctaagtccttcagcaagtcgaaagacacgatgtgagcctatttt

SEQ ID NO: 651 - RNA, 153 nt, synthetic construct
  source 1..153: mol_type = other RNA
ggacctccaggtgcagaagtgcctcgatcaaccgatagaaatatcggtccggtcgaggagagcatcggtctgaaggatgcactccgggatagggcagtcccggctcttgctaagtccttcagcaagtcgaaagacacgatgtgagcctatttt

SEQ ID NO: 652 - RNA, 150 nt, synthetic construct
  source 1..150: mol_type = other RNA
ggacctccaggtgcagaagtgtctcaatcaaccgatagaaatatcggtccggttgagaagagcatcggctgaaggatgcactccgggatagggcagtcccggctcttgctaagtccttcagcaagcgaaagcacgatgtgagcctatttt

SEQ ID NO: 653 - RNA, 146 nt, synthetic construct
  source 1..146: mol_type = other RNA
ggacctccaggtgcagaagtgtctcaatcaaccgagaaatcggtccggttgaaaagagcatcggctgaaggatgcactccgggatagggcagtcccggctcttgctaagtccttcagcaagcgaaagcacgatgtgagcctatttt

SEQ ID NO: 654 - RNA, 153 nt, synthetic construct
  source 1..153: mol_type = other RNA
ctgtgaagagccagtggccagcctcaatcaaccgatagaaatatcggtccggttgaggagagcatcggtctgaaggatgcactccgggatagggcagtcccggctcttgctaagtccttcagcaagtcgaaagacacgatgtgagcctatttt

SEQ ID NO: 655 - RNA, 153 nt, synthetic construct
  source 1..153: mol_type = other RNA
ctgtgaagagccagtggccagcctcgatcaaccgatagaaatatcggtccggtcgaggagagcatcggtctgaaggatgcactccgggatagggcagtcccggctcttgctaagtccttcagcaagtcgaaagacacgatgtgagcctatttt

SEQ ID NO: 656 - RNA, 150 nt, synthetic construct
  source 1..150: mol_type = other RNA
ctgtgaagagccagtggccagtctcaatcaaccgatagaaatatcggtccggttgagaagagcatcggctgaaggatgcactccgggatagggcagtcccggctcttgctaagtccttcagcaagcgaaagcacgatgtgagcctatttt

SEQ ID NO: 657 - RNA, 146 nt, synthetic construct
  source 1..146: mol_type = other RNA
ctgtgaagagccagtggccagtctcaatcaaccgagaaatcggtccggttgaaaagagcatcggctgaaggatgcactccgggatagggcagtcccggctcttgctaagtccttcagcaagcgaaagcacgatgtgagcctatttt

SEQ ID NO: 658 - RNA, 153 nt, synthetic construct
  source 1..153: mol_type = other RNA
agccacgtctacagcagggcgtttcaatcaaccgatagaaatatcggtccggttgaaaagagcatcggtctgaaggatgcactccgggatagggcagtcccggctcttgctaagtccttcagcaagtcgaaagacacgatgtgagcctatttt

SEQ ID NO: 659 - RNA, 153 nt, synthetic construct
  source 1..153: mol_type = other RNA
acagcagggctgcctcggacgtttcaatcaaccgatagaaatatcggtccggttgaaaagagcatcggtctgaaggatgcactccgggatagggcagtcccggctcttgctaagtccttcagcaagtcgaaagacacgatgtgagcctatttt

SEQ ID NO: 660 - RNA, 153 nt, synthetic construct
  source 1..153: mol_type = other RNA
gagggatcctgggagcccttgtttcaatcaaccgatagaaatatcggtccggttgaaaagagcatcggtctgaaggatgcactccgggatagggcagtcccggctcttgctaagtccttcagcaagtcgaaagacacgatgtgagcctatttt

SEQ ID NO: 661 - RNA, 153 nt, synthetic construct
  source 1..153: mol_type = other RNA
agccacgtctacagcagggcgtctcaatcaaccgatagaaatatcggtccggttgagaagagcatcggtctgaaggatgcactccgggatagggcagtcccggctcttgctaagtccttcagcaagtcgaaagacacgatgtgagcctatttt

SEQ ID NO: 662 - RNA, 153 nt, synthetic construct
  source 1..153: mol_type = other RNA
acagcagggctgcctcggacgtctcaatcaaccgatagaaatatcggtccggttgagaagagcatcggtctgaaggatgcactccgggatagggcagtcccggctcttgctaagtccttcagcaagtcgaaagacacgatgtgagcctatttt

SEQ ID NO: 663 - RNA, 153 nt, synthetic construct
  source 1..153: mol_type = other RNA
gagggatcctgggagcccttgtctcaatcaaccgatagaaatatcggtccggttgagaagagcatcggtctgaaggatgcactccgggatagggcagtcccggctcttgctaagtccttcagcaagtcgaaagacacgatgtgagcctatttt

SEQ ID NO: 664 - RNA, 157 nt, synthetic construct
  source 1..157: mol_type = other RNA
ttacagccacgtctacagcagggcgtttcaatcaaccgatagaaatatcggtccggttgaaaagagcatcggtctgaaggatgcactccgggatagggcagtcccggctcttgctaagtccttcagcaagtcgaaagacacgatgtgagcctatttt

SEQ ID NO: 665 - RNA, 157 nt, synthetic construct
  source 1..157: mol_type = other RNA
gtctacagcagggctgcctcggacgtttcaatcaaccgatagaaatatcggtccggttgaaaagagcatcggtctgaaggatgcactccgggatagggcagtcccggctcttgctaagtccttcagcaagtcgaaagacacgatgtgagcctatttt

SEQ ID NO: 666 - RNA, 157 nt, synthetic construct
  source 1..157: mol_type = other RNA
ctctgagggatcctgggagcccttgtttcaatcaaccgatagaaatatcggtccggttgaaaagagcatcggtctgaaggatgcactccgggatagggcagtcccggctcttgctaagtccttcagcaagtcgaaagacacgatgtgagcctatttt

SEQ ID NO: 667 - RNA, 157 nt, synthetic construct
  source 1..157: mol_type = other RNA
ttacagccacgtctacagcagggcgtctcaatcaaccgatagaaatatcggtccggttgagaagagcatcggtctgaaggatgcactccgggatagggcagtcccggctcttgctaagtccttcagcaagtcgaaagacacgatgtgagcctatttt

SEQ ID NO: 668 - RNA, 157 nt, synthetic construct
  source 1..157: mol_type = other RNA
gtctacagcagggctgcctcggacgtctcaatcaaccgatagaaatatcggtccggttgagaagagcatcggtctgaaggatgcactccgggatagggcagtcccggctcttgctaagtccttcagcaagtcgaaagacacgatgtgagcctatttt

SEQ ID NO: 669 - RNA, 157 nt, synthetic construct
  source 1..157: mol_type = other RNA
ctctgagggatcctgggagcccttgtctcaatcaaccgatagaaatatcggtccggttgagaagagcatcggtctgaaggatgcactccgggatagggcagtcccggctcttgctaagtccttcagcaagtcgaaagacacgatgtgagcctatttt

SEQ ID NO: 670 - RNA, 153 nt, synthetic construct
  source 1..153: mol_type = other RNA
ctccctatcagtgatagagagtttcaatcaaccgatagaaatatcggtccggttgaaaagagcatcggtctgaaggatgcactccgggatagggcagtcccggctcttgctaagtccttcagcaagtcgaaagacacgatgtgagcctatttt

SEQ ID NO: 671 - RNA, 153 nt, synthetic construct
  source 1..153: mol_type = other RNA
ctccctatcagtgatagagagtctcaatcaaccgatagaaatatcggtccggttgagaagagcatcggtctgaaggatgcactccgggatagggcagtcccggctcttgctaagtccttcagcaagtcgaaagacacgatgtgagcctatttt

SEQ ID NO: 672 - RNA, 150 nt, synthetic construct
  source 1..150: mol_type = other RNA
agccacgtctacagcagggcgtctcaatcaaccgatagaaatatcggtccggttgagaagagcatcggctgaaggatgcactccgggatagggcagtcccggctcttgctaagtccttcagcaagcgaaagcacgatgtgagcctatttt

SEQ ID NO: 673 - RNA, 150 nt, synthetic construct
  source 1..150: mol_type = other RNA
acagcagggctgcctcggacgtctcaatcaaccgatagaaatatcggtccggttgagaagagcatcggctgaaggatgcactccgggatagggcagtcccggctcttgctaagtccttcagcaagcgaaagcacgatgtgagcctatttt

SEQ ID NO: 674 - RNA, 150 nt, synthetic construct
  source 1..150: mol_type = other RNA
gagggatcctgggagcccttgtctcaatcaaccgatagaaatatcggtccggttgagaagagcatcggctgaaggatgcactccgggatagggcagtcccggctcttgctaagtccttcagcaagcgaaagcacgatgtgagcctatttt

SEQ ID NO: 675 - RNA, 150 nt, synthetic construct; identical to SEQ ID NO: 672
  source 1..150: mol_type = other RNA

SEQ ID NO: 676 - RNA, 150 nt, synthetic construct; identical to SEQ ID NO: 673
  source 1..150: mol_type = other RNA

SEQ ID NO: 677 - RNA, 150 nt, synthetic construct; identical to SEQ ID NO: 674
  source 1..150: mol_type = other RNA

SEQ ID NO: 678 - RNA, 153 nt, synthetic construct; identical to SEQ ID NO: 661
  source 1..153: mol_type = other RNA

SEQ ID NO: 679 - RNA, 153 nt, synthetic construct; identical to SEQ ID NO: 662
  source 1..153: mol_type = other RNA

SEQ ID NO: 680 - RNA, 153 nt, synthetic construct; identical to SEQ ID NO: 663
  source 1..153: mol_type = other RNA

SEQ ID NO: 681 - RNA, 157 nt, synthetic construct; identical to SEQ ID NO: 667
  source 1..157: mol_type = other RNA

SEQ ID NO: 682 - RNA, 157 nt, synthetic construct; identical to SEQ ID NO: 668
  source 1..157: mol_type = other RNA

SEQ ID NO: 683 - RNA, 157 nt, synthetic construct; identical to SEQ ID NO: 669
  source 1..157: mol_type = other RNA

SEQ ID NO: 684 - RNA, 153 nt, synthetic construct; identical to SEQ ID NO: 611
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 685 - RNA, 153 nt, synthetic construct; identical to SEQ ID NO: 612
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 686 - RNA, 153 nt, synthetic construct; identical to SEQ ID NO: 613
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 687 - RNA, 153 nt, synthetic construct; identical to SEQ ID NO: 614
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 688 - RNA, 153 nt, synthetic construct; identical to SEQ ID NO: 615
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 689 - RNA, 153 nt, synthetic construct; identical to SEQ ID NO: 616
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 690 - RNA, 153 nt, synthetic construct; identical to SEQ ID NO: 617
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 691 - RNA, 153 nt, synthetic construct; identical to SEQ ID NO: 618
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 692 - RNA, 153 nt, synthetic construct; identical to SEQ ID NO: 619
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 693 - RNA, 153 nt, synthetic construct; identical to SEQ ID NO: 620
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 694 - RNA, 153 nt, synthetic construct; identical to SEQ ID NO: 621
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 695 - RNA, 153 nt, synthetic construct; identical to SEQ ID NO: 622
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 696 - RNA, 153 nt, synthetic construct; identical to SEQ ID NO: 623
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 697 - RNA, 151 nt, synthetic construct; identical to SEQ ID NO: 624
  source 1..151: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 698 - RNA, 149 nt, synthetic construct; identical to SEQ ID NO: 625
  source 1..149: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 699 - RNA, 152 nt, synthetic construct; identical to SEQ ID NO: 626
  source 1..152: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 700 - RNA, 151 nt, synthetic construct; identical to SEQ ID NO: 627
  source 1..151: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 701 - RNA, 153 nt, synthetic construct; identical to SEQ ID NO: 628
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 702 - RNA, 153 nt, synthetic construct; identical to SEQ ID NO: 629
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 703 - RNA, 153 nt, synthetic construct; identical to SEQ ID NO: 630
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 704 - RNA, 153 nt, synthetic construct; identical to SEQ ID NO: 631
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 705 - RNA, 153 nt, synthetic construct; identical to SEQ ID NO: 632
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 706 - RNA, 151 nt, synthetic construct; identical to SEQ ID NO: 633
  source 1..151: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 707 - RNA, 149 nt, synthetic construct; identical to SEQ ID NO: 634
  source 1..149: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 708 - RNA, 152 nt, synthetic construct; identical to SEQ ID NO: 635
  source 1..152: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 709 - RNA, 151 nt, synthetic construct; identical to SEQ ID NO: 636
  source 1..151: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 710 - RNA, 153 nt, synthetic construct; identical to SEQ ID NO: 637
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 711 - RNA, 153 nt, synthetic construct; identical to SEQ ID NO: 638
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 712 - RNA, 153 nt, synthetic construct; identical to SEQ ID NO: 639
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 713 - RNA, 153 nt, synthetic construct; identical to SEQ ID NO: 640
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 714 - RNA, 153 nt, synthetic construct; identical to SEQ ID NO: 641
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 715 - RNA, 151 nt, synthetic construct; identical to SEQ ID NO: 642
  source 1..151: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 716 - RNA, 149 nt, synthetic construct; identical to SEQ ID NO: 643
  source 1..149: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 717 - RNA, 152 nt, synthetic construct; identical to SEQ ID NO: 644
  source 1..152: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 718 - RNA, 151 nt, synthetic construct; identical to SEQ ID NO: 645
  source 1..151: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 719 - RNA, 153 nt, synthetic construct; identical to SEQ ID NO: 646
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 720 - RNA, 153 nt, synthetic construct; identical to SEQ ID NO: 647
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 721 - RNA, 150 nt, synthetic construct; identical to SEQ ID NO: 648
  source 1..150: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 722 - RNA, 146 nt, synthetic construct; identical to SEQ ID NO: 649
  source 1..146: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 723 - RNA, 153 nt, synthetic construct; identical to SEQ ID NO: 650
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 724 - RNA, 153 nt, synthetic construct; identical to SEQ ID NO: 651
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 725 - RNA, 150 nt, synthetic construct; identical to SEQ ID NO: 652
  source 1..150: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 726 - RNA, 146 nt, synthetic construct; identical to SEQ ID NO: 653
  source 1..146: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 727 - RNA, 153 nt, synthetic construct; identical to SEQ ID NO: 654
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 728 - RNA, 153 nt, synthetic construct; identical to SEQ ID NO: 655
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 729 - RNA, 150 nt, synthetic construct; identical to SEQ ID NO: 656
  source 1..150: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 730 - RNA, 146 nt, synthetic construct; identical to SEQ ID NO: 657
  source 1..146: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 731 - RNA, 153 nt, synthetic construct; identical to SEQ ID NO: 658
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 732 - RNA, 153 nt, synthetic construct; identical to SEQ ID NO: 659
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 733 - RNA, 153 nt, synthetic construct; identical to SEQ ID NO: 660
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 734 - RNA, 153 nt, synthetic construct; identical to SEQ ID NO: 661
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 735 - RNA, 153 nt, synthetic construct; identical to SEQ ID NO: 662
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 736 - RNA, 153 nt, synthetic construct; identical to SEQ ID NO: 663
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 737 - RNA, 157 nt, synthetic construct; identical to SEQ ID NO: 664
  source 1..157: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 738 - RNA, 157 nt, synthetic construct; identical to SEQ ID NO: 665
  source 1..157: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 739 - RNA, 157 nt, synthetic construct; identical to SEQ ID NO: 666
  source 1..157: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 740 - RNA, 157 nt, synthetic construct; identical to SEQ ID NO: 667
  source 1..157: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 741 - RNA, 157 nt, synthetic construct; identical to SEQ ID NO: 668
  source 1..157: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 742 - RNA, 157 nt, synthetic construct; identical to SEQ ID NO: 669
  source 1..157: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 743 - RNA, 153 nt, synthetic construct; identical to SEQ ID NO: 670
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 744 - RNA, 153 nt, synthetic construct; identical to SEQ ID NO: 671
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 745 - RNA, 150 nt, synthetic construct; identical to SEQ ID NO: 672
  source 1..150: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 746 - RNA, 150 nt, synthetic construct; identical to SEQ ID NO: 673
  source 1..150: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 747 - RNA, 150 nt, synthetic construct; identical to SEQ ID NO: 674
  source 1..150: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage

SEQ ID NO: 748 - RNA, 150 nt, synthetic construct; identical to SEQ ID NO: 672
  source 1..150: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 147: mod_base = OTHER; note = Nucleotide with phosphorothioate linkage
  modified_base 148: mod_base = OTHER; note = 2'-O-methyl modified nucleotide with phosphorothioate linkage
  modified_base 149: mod_base = OTHER; note = 2'-O-methyl modified nucleotide with phosphorothioate linkage
  modified_base 150: mod_base = OTHER; note = 2'-O-methyl modified nucleotide

SEQ ID NO: 749 - RNA, 150 nt, synthetic construct; identical to SEQ ID NO: 673
  source 1..150: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 147: mod_base = OTHER; note = Nucleotide with phosphorothioate linkage
  modified_base 148: mod_base = OTHER; note = 2'-O-methyl modified nucleotide with phosphorothioate linkage
  modified_base 149: mod_base = OTHER; note = 2'-O-methyl modified nucleotide with phosphorothioate linkage
  modified_base 150: mod_base = OTHER; note = 2'-O-methyl modified nucleotide

SEQ ID NO: 750 - RNA, 148 nt, synthetic construct
  source 1..148: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 145: mod_base = OTHER; note = Nucleotide with phosphorothioate linkage
  modified_base 146: mod_base = OTHER; note = 2'-O-methyl modified nucleotide with phosphorothioate linkage
  modified_base 147: mod_base = OTHER; note = 2'-O-methyl modified nucleotide with phosphorothioate linkage
  modified_base 148: mod_base = OTHER; note = 2'-O-methyl modified nucleotide
gagggatcctgggagcccttgtctcaatcaaccgatagaaatatcggtccggttgagaagagcatcggctgaaggatgcactccgggatagggcagtcccggctcttgctaagtccttcagcaagcgaaagcacgatgtgagcctttt

SEQ ID NO: 751 - RNA, 153 nt, synthetic construct; identical to SEQ ID NO: 661
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 150: mod_base = OTHER; note = Nucleotide with phosphorothioate linkage
  modified_base 151: mod_base = OTHER; note = 2'-O-methyl modified nucleotide with phosphorothioate linkage
  modified_base 152: mod_base = OTHER; note = 2'-O-methyl modified nucleotide with phosphorothioate linkage
  modified_base 153: mod_base = OTHER; note = 2'-O-methyl modified nucleotide

SEQ ID NO: 752 - RNA, 153 nt, synthetic construct; identical to SEQ ID NO: 662
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 150: mod_base = OTHER; note = Nucleotide with phosphorothioate linkage
  modified_base 151: mod_base = OTHER; note = 2'-O-methyl modified nucleotide with phosphorothioate linkage
  modified_base 152: mod_base = OTHER; note = 2'-O-methyl modified nucleotide with phosphorothioate linkage
  modified_base 153: mod_base = OTHER; note = 2'-O-methyl modified nucleotide

SEQ ID NO: 753 - RNA, 153 nt, synthetic construct; identical to SEQ ID NO: 663
  source 1..153: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 150: mod_base = OTHER; note = Nucleotide with phosphorothioate linkage
  modified_base 151: mod_base = OTHER; note = 2'-O-methyl modified nucleotide with phosphorothioate linkage
  modified_base 152: mod_base = OTHER; note = 2'-O-methyl modified nucleotide with phosphorothioate linkage
  modified_base 153: mod_base = OTHER; note = 2'-O-methyl modified nucleotide

SEQ ID NO: 754 - RNA, 157 nt, synthetic construct; identical to SEQ ID NO: 667
  source 1..157: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 154: mod_base = OTHER; note = Nucleotide with phosphorothioate linkage
  modified_base 155: mod_base = OTHER; note = 2'-O-methyl modified nucleotide with phosphorothioate linkage
  modified_base 156: mod_base = OTHER; note = 2'-O-methyl modified nucleotide with phosphorothioate linkage
  modified_base 157: mod_base = OTHER; note = 2'-O-methyl modified nucleotide

SEQ ID NO: 755 - RNA, 157 nt, synthetic construct; identical to SEQ ID NO: 668
  source 1..157: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 154: mod_base = OTHER; note = Nucleotide with phosphorothioate linkage
  modified_base 155: mod_base = OTHER; note = 2'-O-methyl modified nucleotide with phosphorothioate linkage
  modified_base 156: mod_base = OTHER; note = 2'-O-methyl modified nucleotide with phosphorothioate linkage
  modified_base 157: mod_base = OTHER; note = 2'-O-methyl modified nucleotide

SEQ ID NO: 756 - RNA, 157 nt, synthetic construct; identical to SEQ ID NO: 669
  source 1..157: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 154: mod_base = OTHER; note = Nucleotide with phosphorothioate linkage
  modified_base 155: mod_base = OTHER; note = 2'-O-methyl modified nucleotide with phosphorothioate linkage
  modified_base 156: mod_base = OTHER; note = 2'-O-methyl modified nucleotide with phosphorothioate linkage
  modified_base 157: mod_base = OTHER; note = 2'-O-methyl modified nucleotide

SEQ ID NO: 757 - DNA, 56 bp, synthetic construct
  source 1..56: mol_type = other DNA
acactctttccctacacgacgctcttccgatcttttcactgacatttctcttgtct

SEQ ID NO: 758 - DNA, 57 bp, synthetic construct
  source 1..57: mol_type = other DNA
gtgactggagttcagacgtgtgctcttccgatctgtgtgtctgaatctaacacgaat

SEQ ID NO: 759 - DNA, 183 bp, synthetic construct
  source 1..183: mol_type = other DNA
  misc_difference 1..25: note = a, c, t, g, unknown or other
  misc_difference 1..25: note = This region may encompass 18-25 nucleotides
nnnnnnnnnnnnnnnnnnnnnnnnngtttcaatcaaccgatagaaatatcggtccggttgaaaagagcatcggtctgaaggatgcactccgggatagggcagtcccggctcttgctgtttccccggtaagacctcggaagcaagtccttcagcaagtcgaaagacacgatgtgagcctatttt

SEQ ID NO: 760 - DNA, 209 bp, synthetic construct
  source 1..209: mol_type = other DNA
  misc_difference 1..25: note = a, c, t, g, unknown or other
  misc_difference 1..25: note = This region may encompass 18-25 nucleotides
nnnnnnnnnnnnnnnnnnnnnnnnngttttagtcgaaaggctggagcgttctttggcaattcgaccatgtttccaacaggttgcggctaaaaagagccacgaccgcacctcggcaccctgggatcggacggacagtccccggccctgctgctcggatgcttcatgcgcccgacaaacgacgcggtctatgtggtgagtccgtatttttt

SEQ ID NO: 761 - DNA, 196 bp, synthetic construct
  source 1..196: mol_type = other DNA
  misc_difference 1..25: note = a, c, t, g, unknown or other
  misc_difference 1..25: note = This region may encompass 18-25 nucleotides
nnnnnnnnnnnnnnnnnnnnnnnnngtttcaatcaaactgatttgaaaaaatcagttccggttgaaaagagcatccgtctgaagggcactccgggatagggcagtcccggctcttgctgtttccctggcacatcactgtgctccggaaatgaaccctcggacaagtcgaaagacaggatgtgagcctaatttattt

SEQ ID NO: 762 - DNA, 175 bp, synthetic construct
  source 1..175: mol_type = other DNA
  misc_difference 1..25: note = a, c, t, g, unknown or other
  misc_difference 1..25: note = This region may encompass 18-25 nucleotides
nnnnnnnnnnnnnnnnnnnnnnnnngtttcaggcgacacgaagaaattcgtgtggtgtctgaaaagagccacgtccaggagtccggcacccgtggattggacagtctacggccctgcacggccggtatagagaagccggccaaacggccctggccaatgtggtgagtccattttt

SEQ ID NO: 763 - DNA, 190 bp, synthetic construct
  source 1..190: mol_type = other DNA
  misc_difference 1..25: note = a, c, t, g, unknown or other
  misc_difference 1..25: note = This region may encompass 18-25 nucleotides
nnnnnnnnnnnnnnnnnnnnnnnnngtttcaatcaagctgaagaaattcagctccggttgaaaagagcatccgtctggtagccatgcactccggaatggggcagttccggctcttgcgacttagtgggtgaaagctcattgagccaactacgagacacgtctctacgagacaggatgtgagcccttattt

SEQ ID NO: 764 - DNA, 199 bp, synthetic construct
  source 1..199: mol_type = other DNA
  misc_difference 1..25: note = a, c, t, g, unknown or other
  misc_difference 1..25: note = This region may encompass 18-25 nucleotides
nnnnnnnnnnnnnnnnnnnnnnnnnctttcactctagcgaaagctagagtgaaagaagcccaggcgctgctccagtcctcgccgatgtaacccagcatcggaaccagggtgtgggcatccccgtaggccggtactcggaccccggcaaagggcaagggttggtggcgcactcgacgagcgaggagcgccacctcatttt

SEQ ID NO: 765 - DNA, 184 bp, synthetic construct
  source 1..184: mol_type = other DNA
  misc_difference 1..25: note = a, c, t, g, unknown or other
  misc_difference 1..25: note = This region may encompass 18-25 nucleotides
nnnnnnnnnnnnnnnnnnnnnnnnngtttcaatcaagctgagaaatcagctccggttgaaaagagcatccgtctggcgggcactccggaatggggcagttccggctcttgcagtccagtgagttcattcgcattgaaccaactgccaggcacgtcttcggacaggatgtgagcccgtatttctt

SEQ ID NO: 766 - DNA, 176 bp, synthetic construct
  source 1..176: mol_type = other DNA
  misc_difference 1..25: note = a, c, t, g, unknown or other
  misc_difference 1..25: note = This region may encompass 18-25 nucleotides
nnnnnnnnnnnnnnnnnnnnnnnnngttttggtcagccgaaaggttgcggccaaaaagagccacgaccgctattcggcacccggggatcggacggacagtccccggccctgcggatcgggcgtttcgcacgtccgacaaacgacgcggtccatgtggtgagtccgttttatttttt

SEQ ID NO: 767 - DNA, 217 bp, synthetic construct
  source 1..217: mol_type = other DNA
  misc_difference 1..25: note = a, c, t, g, unknown or other
  misc_difference 1..25: note = This region may encompass 18-25 nucleotides
nnnnnnnnnnnnnnnnnnnnnnnnnctttcactctagcgaaagctagagtgaaagaagcccaggcgcctccgttctcctcgctgtaacccagcagcgggtttaaggcataggaggccccgcttccaggctctcggctcgcctggaactcccgcaagggagaagggcaagggtcgcgacgccgctggccgggcagaaggcgtcgccaaatcgctcttt

SEQ ID NO: 768 - DNA, 182 bp, synthetic construct
  source 1..182: mol_type = other DNA
  misc_difference 1..25: note = a, c, t, g, unknown or other
  misc_difference 1..25: note = This region may encompass 18-25 nucleotides
nnnnnnnnnnnnnnnnnnnnnnnnngtttcagtcaccctgaacgaaagttcgggttcaggctgaaaagagcatctgtccggaagggccactccgggttagggcagatccggcacttgggcctccttggtctttttgactctggaggaaacctttcggcgagtcttcggacaagatgcgagcc

SEQ ID NO: 769 - DNA, 176 bp, synthetic construct
  source 1..176: mol_type = other DNA
  misc_difference 1..25: note = a, c, t, g, unknown or other
  misc_difference 1..25: note = This region may encompass 18-25 nucleotides
nnnnnnnnnnnnnnnnnnnnnnnnngtttcaggcaacatcgaaagatgtcgcgtctgaaaagagcctagacgcgagccagcacccaggaatgcggacagttcctggccctgcatgacggtcatagagaagactgtcgaaatggctctcgtccataatggtgagtccatattatttt

SEQ ID NO: 770 - DNA, 194 bp, synthetic construct
  source 1..194: mol_type = other DNA
  misc_difference 1..25: note = a, c, t, g, unknown or other
  misc_difference 1..25: note = This region may encompass 18-25 nucleotides
nnnnnnnnnnnnnnnnnnnnnnnnngtttcaatcaaactgatgaaaatcagttccggttgaaaagagcatccgtccggagggtgcactccgggatggggcagtcccggcacttgcgttttccccggcttacgcttcggaaaaaggcccttcggcacgtcgaaagacaggatgtgagcccaatttaaatattgtt

SEQ ID NO: 771 - DNA, 251 bp, synthetic construct
  source 1..251: mol_type = other DNA
  misc_difference 1..25: note = a, c, t, g, unknown or other
  misc_difference 1..25: note = This region may encompass 18-25 nucleotides
nnnnnnnnnnnnnnnnnnnnnnnnnctttcagaatgctcgaaagagcattctgaaagaagcccagacgcctcgcgcgcccgcaggtagcccagcttgcggcgtctgtcgtatcggtggggtgagacagacattaaccgcacccacctgcaaataggcgaggcctcaccggggaggttcggcggaccccggcgccactggcgagggcaagggtagagaacgctgcatccgaacctctgcagcgttcgctttt

SEQ ID NO: 772 - RNA, 133 nt, synthetic construct
  source 1..133: mol_type = other RNA
ggttcaatcaaccgatagaaatatcggtccggttgaacagagcatcggtctgaaggatgcactccgggatagggcagtcccggctcttgctaagtccttcagcaagtcgaaagacacgatgtgagcctatttt

SEQ ID NO: 773 - protein, 5 aa, synthetic construct
GGGGS

SEQ ID NO: 774 - RNA, 183 nt, synthetic construct
  source 1..183: mol_type = other RNA
  misc_difference 1..25: note = a, c, u, g, unknown or other
  misc_difference 1..25: note = This region may encompass 15-25 nucleotides
nnnnnnnnnnnnnnnnnnnnnnnnngtttcaatcaaccgatagaaatatcggtccggttgaaaagagcatcggtctgaaggatgcactccgggatagggcagtcccggctcttgctgtttccccggtaagacctcggaagcaagtccttcagcaagtcgaaagacacgatgtgagcctatttt

(19) United States
(12) Patent Application Publication
(10) Pub. No.: US 2026/0317759 A1
(43) Pub. Date: Oct. 15, 2026

(54) MODIFIED CAS9-IID NUCLEASES AND USES THEREOF

(71) Applicant: AERA THERAPEUTICS, INC., Cambridge, MA (US)

(72) Inventors: Alexander THOMAS, Cambridge, MA (US); Neena PYZOCHA, Cambridge, MA (US); Hannah KEMPTON, Cambridge, MA (US); Liyang ZHANG, Cambridge, MA (US); Yuanyuan GAO, Cambridge, MA (US); Ming SUN, Cambridge, MA (US); Sean BENLER, Cambridge, MA (US); Ian SLAYMAKER, Cambridge, MA (US)

(73) Assignee: AERA THERAPEUTICS, INC., Cambridge, MA (US)

(21) Appl. No.: 19/481,320

(22) PCT Filed: May 3, 2024
(86) PCT No.: PCT/US2024/027776

§ 371 Date: Nov. 4, 2025

Related U.S. Application Data
(60) Provisional application No. 63/464,439, filed on May 5, 2023.
(60) Provisional application No. 63/533,008, filed on Aug. 16, 2023.

(51) Int. Cl. C12N 15/11; C12N 9/22

(52) U.S. Cl. CPC C12N 15/111; C12N 9/226; C12N 2310/20; C12N 2310/315; C12N 2310/321; C12N 2310/322; C12N 2310/531; C12N 2800/22

(57) ABSTRACT

Described herein are systems, methods, and compositions capable of targeting nucleic acids. Describe in certain exemplary embodiments herein are a class of small Cas proteins (Type II-D Cas proteins) and systems thereof. Also described in certain exemplary embodiments herein are methods of modifying target sequences using the class of small Cas proteins (Type II-D Cas proteins) and systems thereof described herein.

### CROSS-REFERENCE TO RELATED APPLICATIONS

This application is a 35 U.S.C. § 371 National Phase Entry Application of International Patent Application No. PCT/US2024/027776 filed on May 3, 2024, which designated the U.S., and which claims benefit under 35 U.S.C. § 119(e) of U.S. Provisional Application No. 63/464,439 filed May 5, 2023, and U.S. Provisional Application No. 63/533,008 filed Aug. 16, 2023, the contents of each of which are incorporated herein by reference in their entireties.

### SEQUENCE LISTING

The instant application contains a Sequence Listing which has been submitted in XML format via Patent Center and is hereby incorporated by reference in its entirety. Said XML copy, created on Aug. 9, 2024, is named 098791-000103WOPT_SL.xml and is 934,386 bytes in size.

### TECHNICAL FIELD

The technology described herein relates to modified Cas9-IID nucleases, engineered guide RNAs, and uses thereof.

### BACKGROUND

Certain prokaryotes (some bacteria and most archaea) display primitive adaptive immunity against bacteriophage infections and can eliminate the invading genetic material. The CRISPR/Cas system is an example of such a prokaryotic immune system. Clustered regularly interspaced short palindromic repeats (CRISPR) are segments of prokaryotic DNA containing short, repetitive base sequences (for example, up to 100 identical repeats of 25-40 base pairs). Each CRISPR repeat sequence is followed by short segments of interspersed exogenous “spacer” DNA from previous “infections”, i.e., exposure to viruses, phage, or plasmids. CRISPR clusters are transcribed as multi-unit precursors that are subsequently cleaved into smaller units and processed to form guide CRISPR RNAs (guide RNA) that consist of one spacer flanked by sequence derived from a CRISPR repeat. CRISPR loci also contain one or more genes encoding Cas proteins. The guide RNA harboring the spacer sequence directs Cas proteins to exogenous invading DNA and allows the enzyme to cleave it, thereby conferring a type of resistance against the invader. DNA is recognized for cleavage not only by its homology to a spacer sequence of the CRISPR cluster, but also by its proximity to a protospacer adjacent motif (PAM), a sequence that is typically 2-6 nucleotides in length.

Recent application of advances in genome sequencing technologies and analysis have yielded significant insights into the genetic underpinning of biological activities in many diverse areas of nature, ranging from prokaryotic biosynthetic pathways to human pathologies. To fully understand and evaluate the vast quantities of information produced by genetic sequencing technologies, equivalent increases in the scale, efficacy, and ease of technologies for genome and epigenome manipulation are needed. These novel genome and epigenome engineering technologies will accelerate the development of novel applications in numerous areas, including biotechnology, agriculture, and human therapeutics.

Clustered Regularly Interspaced Short Palindromic Repeats (CRISPR) and the CRISPR-associated (Cas) genes, collectively known as the CRISPR-Cas or CRISPR/Cas systems, are currently understood to provide immunity to bacteria and archaea against phage infection. The DNA and RNA systems of prokaryotic adaptive immunity are an extremely diverse group of proteins effectors, non-coding elements, as well as loci architectures, some examples of which have been engineered and adapted to produce important biotechnologies.

The components of the system involved in host defense include one or more effector proteins capable of modifying DNA or RNA and an RNA guide element that is responsible to targeting these protein activities to a specific sequence on the phage DNA or RNA. The RNA guide is composed of a CRISPR RNA (crRNA) and may require an additional trans-activating RNA (tracrRNA) to enable targeted nucleic acid manipulation by the effector protein(s). The crRNA consists of a direct repeat responsible for protein binding to the crRNA and a spacer sequence that is complementary to the desired nucleic acid target sequence. CRISPR systems can be reprogrammed to target alternative DNA or RNA targets by modifying the spacer sequence of the crRNA.

DNA and RNA systems can be broadly classified into two classes: Class 1 systems, which are composed of multiple effector proteins that together form a complex around a crRNA, and Class 2 systems, which consist of a single effector protein that complexes with the RNA guide to target DNA or RNA substrates. The single-subunit effector composition of the Class 2 systems provides a simpler component set for engineering and application translation and have thus far been an important source of programmable effectors. Thus, the discovery, engineering, and optimization of novel Class 2 systems may lead to widespread and powerful programmable technologies for genome engineering and beyond.

The characterization and engineering of Class 2 systems, exemplified by CRISPR-Cas9, have paved the way for a diverse array of biotechnology applications in genome editing and beyond. For example, the effector proteins Cas12a (Cpf1) and Cas13a (C2c2) possess non-specific “collateral” single-stranded-nuclease cleavage activities, which may be harnessed to create novel diagnostics, methods, and other applications. Nevertheless, there remains a need for additional programmable effectors and systems for modifying nucleic acids and polynucleotides (i.e., DNA, RNA, or any hybrid, derivative, or modification) beyond the current Cas systems that enable novel applications through their unique properties.

Cas9-IID is an RNA-guided endonuclease with measurable activity in human cells. The RNA component of Cas9-IID (i.e. guide RNA) as a single sequence with 14-25 nt variable region at the 5′-end which guides the endonuclease to the target site by RNA-DNA complementarity, followed by a 178 nt conserved region. To improve the utility of Cas9-IID as gene editing tool in human cells, guide RNAs that are shorter and have higher efficacy are needed. This disclosure answers these needs.

### SUMMARY

Described herein are Cas9-IID enzymes and systems, that are useful for modifying target nucleic acid sequences. Accordingly, provided herein is an engineered or non-naturally occurring Cas9-IID enzymes and systems that include an engineered guide RNA comprising a guide sequence, where the guide sequence is capable of hybridizing with a target sequence of a target nucleic acid molecule.

In various embodiments of engineered or non-naturally occurring Cas9-IID enzymes and systems provided herein the effector polypeptide can comprise: an amino acid sequence selected from the group consisting of SEQ ID NO: 1, SEQ ID NO:2, SEQ ID NO:3, SEQ ID NO:4, SEQ ID NO:5, SEQ ID NO:6, SEQ ID NO:7, SEQ ID NO:8, SEQ ID NO:10, SEQ ID NO: 11, SEQ ID NO: 12 and SEQ ID NO: 13; or a variant of a protein effector comprising an amino acid sequence having at least 60%, 65%, 70%, 75%, 80%, 85%, 90%, or 95% identity to an amino acid sequence selected from the group consisting of SEQ ID NO: 1, SEQ ID NO:2, SEQ ID NO:3, SEQ ID NO:4, SEQ ID NO:5, SEQ ID NO:6, SEQ ID NO:7, SEQ ID NO:8, SEQ ID NO:10, SEQ ID NO: 11, SEQ ID NO: 12 and SEQ ID NO: 13.

In some embodiments, the Cas9-IID protein comprises an amino acid sequence of SEQ ID NO: 1, and wherein the amino acid sequence of the Cas9-IID protein comprises at least one, at least two, at least three, at least four, at least five, at least six, at least seven, at least eight, or at least nine mutation(s) at position(s) 7, 45, 46, 47, 48, 49, 50, 51, 52, 53, 54, 55, 56, 558, 842, 843, 844, 845, 846, 847, 848, 849, 850, 851, 852, 853, 854, 908, 910, 911, 912, 913, 914, 915, 916, 917, 918, 919, 920, and 921 of the amino acid sequence of SEQ ID NO: 1.

In some embodiments, the Cas9-IID protein comprises an amino acid sequence of SEQ ID NO: 1, and wherein the amino acid sequence of the Cas9-IID protein comprises at least one, at least two, at least three, at least four, at least five, at least six, at least seven, at least eight, or at least nine mutation(s) at position(s) 13, 19, 20, 40, 69, 70, 71, 89, 105, 106, 131, 153, 180, 185, 215, 221, 239, 302, 366, 367, 370, 372, 376, 387 410, 458, 469, 473, 495, 537, 538, 571, 598, 609, 610, 611, 657, 700, 736, 737, 786, 800, 821, 827, 828, 843, 866, 873, 901, 913, 928, and 930 of the amino acid sequence of SEQ ID NO: 1.

In some embodiments, the Cas9-IID protein comprises an amino acid sequence of SEQ ID NO: 1, and wherein the amino acid sequence of the Cas9-IID protein comprises at least one, at least two, at least three, at least four, at least five, at least six, at least seven, at least eight, or at least nine mutation(s) selected independently from I13V, L19R, T20N, H40R, Q69K, H70R, H70K, A71R, A71K, S89C, E105K, T106K, E131R, H153K, A180R, L185R, I215V, N221R, S239K, 1302V, S366K, W367R, 1370R, D372K, A376K, M387R, L410K, T458R, H469K, S473K, K495R, T537K, L538R, A571K, L598R, S609V, A610R, S611K, Q657R, N700K, C736R, N737K, Q786R, N800R, A821R, D827K, M828R, I843K, H866K, L873W, A901E, A901K, Q913K, 1928V, and K930R of the amino acid sequence of SEQ ID NO: 1.

In some embodiments, the Cas9-IID protein comprises an amino acid sequence of SEQ ID NO: 1, and wherein the amino acid sequence of the Cas9-IID protein comprises at least one, at least two, at least three, at least four, at least five, at least six, at least seven, at least eight, or at least nine mutation(s) selected independently from L19R, H70K, H70R, A71K, A71R, E131R, H153K, A180R, L185R, N221R, S239K, I302V, S366K, W367R, I370R, D372K, A376K, L410K, S473K, T537K, L538R, N558R, A571K, S609R, S626V, T688K, E689K, N700K, C736R, N737K, Q786R, A821R, D827K, I843K, I843R, L847K, A848R, L853K, Q913K, Q913S, F920K, L363Ins, and K694Ins of the amino acid sequence of SEQ ID NO: 1 (see e.g., Table 2A).

In some embodiments, the Cas9-IID protein comprises an amino acid sequence of SEQ ID NO: 1, and wherein the amino acid sequence of the Cas9-IID protein comprises at least one, at least two, at least three, at least four, at least five, at least six, at least seven, at least eight, or at least nine mutation(s) selected independently from I13V, L19R, H40R, H70K, H70R, A71K, A71R, H153K, A180R, L185R, N221R, S239K, I302V, S366K, W367R, D372K, S473K, T537K, L538R, N558R, S609R, S609V, A610R, S626V, T688K, E689K, N700K, C736R, Q786R, A821R, D827K, I843K, I843R, L847K, A848R, L853K, Q913K, Q913S, S914N, F920K, F920S, I928V, V942K, A695R, L363Ins, and K694Ins of the amino acid sequence of SEQ ID NO: 1 (see e.g., Table 2B).

In some embodiments, the Cas9-IID protein comprises an amino acid sequence of SEQ ID NO: 1, and wherein the amino acid sequence of the Cas9-IID protein comprises at least one, at least two, at least three, at least four, at least five, at least six, at least seven, at least eight, or at least nine mutation(s) selected independently from I13V, L19R, H40R, H70K, H70R, A71K, A71R, E131R, H153K, A180R, L185R, N221R, S239K, I302V, S366K, W367R, I370R, D372K, A376K, L410K, S473K, T537K, L538R, N558R, A571K, S609R, S609V, A610R, S626V, T688K, E689K, N700K, C736R, N737K, Q786R, A821R, D827K, I843K, I843R, L847K, A848R, L853K, Q913K, Q913S, S914N, F920K, F920S, I928V, V942K, A695R, L363Ins, and K694Ins of the amino acid sequence of SEQ ID NO: 1 (see e.g., Tables 2A-2B).

In some embodiments, the Cas9-IID protein comprises an amino acid sequence of SEQ ID NO: 1, and wherein the amino acid sequence of the Cas9-IID protein comprises a mutation selected from the group consisting of A180R, S239K, S366K, D372K, E689K, Q786R, I843K, I843R, L853K, Q913K, L363Ins, or combination thereof.

In some embodiments, the Cas9-IID protein comprises an amino acid sequence of SEQ ID NO: 1, and wherein the amino acid sequence of the Cas9-IID protein comprises a mutation of I843K, I843R, Q913K, or combination thereof.

In some embodiments, the Cas9-IID protein comprises an amino acid sequence of SEQ ID NO: 1 comprising a mutation of A180R.

In some embodiments, the Cas9-IID protein comprises an amino acid sequence of SEQ ID NO: 1 comprising a mutation selected from the group consisting of S239K.

In some embodiments, the Cas9-IID protein comprises an amino acid sequence of SEQ ID NO: 1 comprising a mutation of S366K.

In some embodiments, the Cas9-IID protein comprises an amino acid sequence of SEQ ID NO: 1 comprising a mutation D372K.

In some embodiments, the Cas9-IID protein comprises an amino acid sequence of SEQ ID NO: 1 comprising a mutation of E689K.

In some embodiments, the Cas9-IID protein comprises an amino acid sequence of SEQ ID NO: 1 comprising a mutation of Q786R.

In some embodiments, the Cas9-IID protein comprises an amino acid sequence of SEQ ID NO: 1 comprising a mutation of I843K.

In some embodiments, the Cas9-IID protein comprises an amino acid sequence of SEQ ID NO: 1 comprising a mutation of I843R.

In some embodiments, the Cas9-IID protein comprises an amino acid sequence of SEQ ID NO: 1 comprising a mutation of L853K.

In some embodiments, the Cas9-IID protein comprises an amino acid sequence of SEQ ID NO: 1 comprising a mutation of Q913K.

In some embodiments, the Cas9-IID protein comprises an amino acid sequence of SEQ ID NO: 1 comprising a mutation of L363Ins.

In some embodiments, the Cas9-IID protein comprises an amino acid sequence of SEQ ID NO: 9, and wherein the amino acid sequence of the Cas9-IID protein comprises at least one, at least two, at least three, at least four, at least five, at least six, at least seven, at least eight, or at least nine mutation(s) position(s) 7, 55, 56, 57, 58, 59, 60, 61, 62, 63, 64, 65, 66, 624, 858, 859, 860, 861, 862, 863, 864, 865, 866, 867, 868, 896, 897, 898, 899, 900, 901, 902, 903, and 904 of the amino acid sequence of SEQ ID NO: 9.

In some embodiments, the Cas9-IID protein comprises an amino acid sequence of SEQ ID NO: 12, and wherein the amino acid sequence of the Cas9-IID protein comprises at least one, at least two, at least three, at least four, at least five, at least six, at least seven, at least eight, or at least nine mutation(s) at position(s) 7, 45, 46, 47, 48, 49, 50, 51, 52, 53, 54, 55, 56, 558, 843, 844, 845, 846, 847, 848, 849, 850, 851, 852, 853, 854, 855, 909, 910, 911, 912, 913, 914, 915, 916, 917, 918, 919, 920, 921, and 922 of the amino acid sequence of SEQ ID NO: 12.

In some embodiments, the Cas9-IID protein comprises an amino acid sequence of SEQ ID NO: 12, and wherein the amino acid sequence of the Cas9-IID protein comprises a mutation at least one, at least two, at least three, at least four, at least five, at least six, at least seven, at least eight, or at least nine mutation(s) selected independently from I13V, L19R, T20N, H40R, Q69K, H70R, H70K, A71R, A71K, S89C, E105K, T106K, E131R, H153K, A180R, L185R, I215V, N221R, S239K, 1302V, S366K, W367R, 1370R, D372K, A376K, M387R, L410K, T458R, H469K, S473K, K495R, T537K, L538R, A571K, L598R, S609V, A610R, S611K, Q657R, N700K, C736R, N737K, Q786R, N800R, A821R, D827K, M828R, I843K, H866K, L873W, A901E, A901K, Q913K, I928V, and K930R of the amino acid sequence of SEQ ID NO: 12.

In some embodiments, the nickase of Cas9-IID protein of SEQ ID NO:1 or 13 comprises a mutation at position 7 and/or 558. Preferably the mutations for the nickase Cas9-II are selected from D7A, D7G, N558A, N559G, and combinations thereof.

In some embodiments, the nickase of Cas9-IID protein of SEQ ID NO:9 comprises a mutation at position 7 and/or 624. Preferably the mutations for the nickase Cas9-II are selected from D7A, D7G, R624A, R624G, and combinations thereof.

### BRIEF DESCRIPTION OF THE DRAWINGS

FIG. 1 is a series of bar graphs showing engineered IID-25 guides approaching Cas9 activity in RNA delivery. The left-right order of the legend corresponds to the left-right order of each group of bars.

FIG. 2 is a bar graph showing all RNA delivery to AML12 cell line for V70 (left group of bars) or V118 (right group of bars).

FIG. 3 depicts an exemplary guide RNA secondary structure (Structure A, e.g., SEQ ID NO: 409). In some embodiments, N20 is a spacer sequence of 15-25 nucleotides at the 5′-end, each of S1, S2, S3, S4, S5 and S6 independently comprise 2-20 nucleotide base-pairs. and optionally, the guide RNA is less than 155 nucleotides and comprises at least one modification (e.g., at least one nucleic acid modification. Figure discloses SEQ ID NO: 774.

### DETAILED DESCRIPTION

In some embodiments, the Cas9-IID system comprising: a) a Cas9-IID proteins effector polypeptide, or one or more nucleotide sequences encoding the protein effector polypeptide, wherein the protein effector polypeptide comprises an amino acid sequence SEQ ID NO:9, or comprises a variant of a protein effector comprising an amino acid sequence having at least 60%, 65%, 70%, 75%, 80%, 85%, 90%, or 95% identity to an amino acid sequence selected from the group consisting of SEQ ID NO:9, or comprises the amino acid sequence of a naturally-occurring protein effector having at least 30% identity to an amino acid sequence selected from the group consisting of SEQ ID NO:9, and b) one or more engineered guide RNAs comprising a guide sequence, wherein the one or more guide RNAs is designed to form a complex with the protein effector polypeptide and wherein the one or more guide RNAs comprises a guide sequence designed to hybridize with one or more target nucleic acid molecules, wherein the guide RNA and the protein effector polypeptide do not naturally occur together.

In some embodiments, the engineered, non-naturally occurring Cas9-IID system comprising one or more nucleic acid constructs comprising: a) a polynucleotide sequence encoding a protein effector polypeptide; wherein the protein effector polypeptide comprises an amino acid sequence selected from the group comprising SEQ ID NO:9, or comprises a variant of a protein effector comprising an amino acid sequence having at least 60%, 65%, 70%, 75%, 80%, 85%, 90%, or 95% identity to an amino acid sequence selected from the group consisting of SEQ ID NO:9, or comprises the amino acid sequence of a naturally-occurring protein effector having at least 50% identity to an amino acid sequence selected from the group consisting of SEQ ID NO:9, and b) a polynucleotide sequence encoding a guide RNA, wherein the guide RNA is designed to form a complex with the protein effector polypeptide and wherein the guide RNA comprises a guide sequence designed to hybridize with one or more target nucleic acid molecules, wherein the guide RNA and the protein effector polypeptide do not naturally occur together.

In some embodiments, the Cas9-IID system comprising: a) a Cas9-IID proteins effector polypeptide, or one or more nucleotide sequences encoding the protein effector polypeptide, wherein the protein effector polypeptide comprises an amino acid sequence SEQ ID NO:1, or comprises a variant of a protein effector comprising an amino acid sequence having at least 60%, 65%, 70%, 75%, 80%, 85%, 90%, or 95% identity to an amino acid sequence selected from the group consisting of SEQ ID NO:1, or comprises the amino acid sequence of a naturally-occurring protein effector having at least 70% identity to an amino acid sequence selected from the group consisting of SEQ ID NO: 1, and b) one or more engineered guide RNAs comprising a guide sequence, wherein the one or more guide RNAs is designed to form a complex with the protein effector polypeptide and wherein the one or more guide RNAs comprises a guide sequence designed to hybridize with one or more target nucleic acid molecules, wherein the guide RNA and the protein effector polypeptide do not naturally occur together.

In some embodiments, the Cas9-IID protein comprises an amino acid sequence of SEQ ID NO: 12, and wherein the amino acid sequence of the Cas9-IID protein comprises a mutation at least one, at least two, at least three, at least four, at least five, at least six, at least seven, at least eight, or at least nine mutation(s) selected independently from S11K, W40R, H63R, Q67R, Q67K, E70R, E70K, Q71R, Q71K, S71R, S84K, N90R, E109K, T127K, S131R, Q138K, S144K, T148K, T148R, Q153K, Q153R, L158K, L158R, Q162K, S185R, G190R, A204K, C212R/K, E221R, E224R, N239K, E257K, E277R, T278R, S287K, Q315K, S320R, L363Ins(NKKKSRR, SEQ ID NO: 507), T369K, I370R, Q376K, M387R, E391K, V393R, E399K, Q410K, V418R, H424R, A425R, S444R, D449R, T458R, D469R, S476K, T477K, Q495R, Q541K, A542K, A544K, A568R, A571K, Y593K, L598R, P601R, A609R, D610R, T611K, D621K, D629K, P631R, T639K, S652K, M661K, E672K, G675K, M676K, P696R, S701K, 1738K, A745K, H746K, N751R, A757R, S760K, M781R, Q798K, G801K, D828K, T844K, T844R, I848K, S849R, P854K, D860K, V862R, G872K, N905K, S914K, E916K, S937R, and V943K of the amino acid sequence of SEQ ID NO: 12.

In some embodiments, the Cas9-IID protein comprises an amino acid sequence of SEQ ID NO: 27, and wherein the amino acid sequence of the Cas9-IID protein comprises a mutation at least one, at least two, at least three, at least four, at least five, at least six, at least seven, at least eight, or at least nine mutation(s) selected independently from V13K, C21R, T36R, E40R, V41R, S64R, N67R, Q71K, A72K, A85K, H88R, E104K, E107K, N108K, G109K, Q110K, A111K, L139K, P142K, H154R, L213R, H214R, D216K, A219R, T223R, E226R, D240K, D264K, V268R, S270R, E271R, G292K, C297R, A308K, L356Ins(NKKKSRR, SEQ ID NO: 507), Q362K, D368R, Q392K, A443R, A465K, F468K, 1470K, Q498R, N505K, Q530K, S535K, Q537K, A539R, E564K, L568K, D581K, T586R, L591R, V602R, A604K, T606R, V607K, A608K, P611K, P614K, E622K, G624R, E632K, G654K, S657K, N666K, N668K, N688K, T689R, S690K, G691K, 1695K, T698K, Q706K, Q709R, D738K, N744R, A750R, D757K, S780R, P813K, L814K, S823R, A843K, P845K, A851K, P858K, D863K, A867K, E875K, T905K, Q907K, N911K, F913R, V918K, A928R, V933K, and P790R of the amino acid sequence of SEQ ID NO: 27.

In some embodiments, the Cas9-IID proteins and related systems and compositions described herein have a target specificity, more particularly the binding of the Cas9-IID proteins-guide complex is PAM-dependent. The Cas9-IID proteins and related systems and compositions described herein can be modified to include PAM specificity (as described in Kleinstiver et al. 2015; Hirano et al. Mol. Cell 2016).

In some embodiments, the present disclosure provides for a method for binding, cleaving, marking, or modifying a double-stranded deoxyribonucleic acid polynucleotide, comprising: (a) contacting said double-stranded deoxyribonucleic acid polynucleotide with a Cas9-IID endonuclease in complex with an engineered guide ribonucleic acid structure configured to bind to said endonuclease and said double-stranded deoxyribonucleic acid polynucleotide; (b) wherein said double-stranded deoxyribonucleic acid polynucleotide comprises a protospacer adjacent motif (PAM); wherein said Cas9-IID endonuclease has a molecular weight of about 120 kDa or less, 100 kDa or less, 90 kDa or less, or 60 kDa or less. In some embodiments, said endonuclease cleaves said double-stranded deoxyribonucleic acid polynucleotide, wherein said PAM comprises NGG, NACC, NVC, NRGM, NAC, NVCCC, NAV, NVC, or NAC. In some embodiments, said endonuclease cleaves said double-stranded deoxyribonucleic acid polynucleotide 6-8 nucleotides or 7 nucleotides from said PAM. In some embodiments, said endonuclease comprises a variant with at least 70%, at least 75%, at least 80% or at least 90% sequence identity to any one of SEQ ID NOs: 1-13.

### Nuclear Localization Sequences

In some embodiments of any one of the aspects described herein, the Cas9-IID protein further comprises a nuclear localization sequence (NLS) sequence or a variant thereof. The NLS can be proximal to the N- or C-terminus of the Cas9-IID protein. The NLS can be linked (e.g., fused) to the N-terminus and/or C-terminus of the Cas9-IID protein, and can be fused singly (i.e., a single NLS) or concatenated e.g., a chain of 2, 3, 4, or more NLS). When more than one NLS is present, each may be selected independently of the others, such that a single NLS may be present in more than one copy and/or in combination with one or more other NLSs present in one or more copies.

In some embodiments, the Cas9-IID protein is fused to one or more nuclear localization sequences (NLSs), such as about or more than about 1, 2, 3, 4, 5, 6, 7, 8, 9, 10, or more NLSs. In some embodiments, the Cas9-IID protein comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g. zero or at least one or more NLS at the amino-terminus and zero or at one or more NLS at the carboxy terminus). Some exemplary NLS include, but are not limited to, those shown in Table A.

TABLE A
Exemplary NLS sequences

| | SEQ ID | |
|---|---|---|
| Sequence | NO | Source |
| PKKKRKV | 29 | SV40 virus large |
| | | T-antigen |
| KRPAATKKAGQAKKKK | 30 | nucleoplasmin |
| | | bipartite NLS |
| PAAKRVKLD | 31 | c-myc NLS |
| RQRRNELKRSP | 32 | c-myc NLS |
| NQSSNFGPMKGGNFGGRSSGPYGGGGQYFAKPRNQGGY | 33 | hRNPA1 M9 NLS |
| RMRIZFKNKGKDTAELRRRRVEVSVELRKAKKDEQILKRRNV | 34 | IBB domain from |
| | | importin-alpha |
| VSRKRPRP | 35 | myoma T protein |
| PPKKARED | 36 | myoma T protein |
| PQPKKKPL | 37 | human p53 |
| SALIKKKKKMAP | 38 | mouse c-abl IV |
| DRLRR | 39 | influenza virus |
| | | NS1 |
| PKQKKRK | 40 | influenza virus |
| | | NS1 |
| RKLKKKIKKL | 41 | Hepatitis virus |
| | | delta antigen |
| REKKKFLKRR | 42 | mouse Mx1 |
| | | protein |
| KRKGDEVDGVDEVAKKKSKK | 43 | human poly(ADP- |
| | | ribose) polymerase |
| RKCLQAGMNLEARKTKK | 44 | human |
| | | glucocorticoid |
| | | receptor |

In some embodiments, the NLS comprises an amino acid sequence having at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity with any one of the NLS sequences show in Table A, or a homolog or ortholog thereof.

### Nuclear Export Signal Sequences

In some embodiments of any one of the aspects described herein, the Cas9-IID protein further comprises a nuclear export signal (NES) sequence or a variant thereof. The NES can be proximal to the N- or C-terminus of the Cas9-IID protein. The NES can be linked (e.g., fused) to the N-terminus and/or C-terminus of the Cas9-IID protein, and can be fused singly (i.e., a single NES) or concatenated e.g., a chain of 2, 3, 4, or more NLS). When more than one NES is present, each may be selected independently of the others, such that a single NES may be present in more than one copy and/or in combination with one or more other NESs present in one or more copies.

In some embodiments, the Cas9-IID protein is fused to one or more NESs, such as about or more than about 1, 2, 3, 4, 5, 6, 7, 8, 9, 10, or more NESs. In some embodiments, the Cas9-IID protein comprises about or more than about 1, 2, 3, 4, 5, 6, 7, 8, 9, 10, or more NESs at or near the amino-terminus, about or more than about 1, 2, 3, 4, 5, 6, 7, 8, 9, 10, or more NESs at or near the carboxy-terminus, or a combination of these (e.g. zero or at least one or more NES at the amino-terminus and zero or at one or more NES at the carboxy terminus).

In some embodiments, a C-terminal and/or N-terminal NLS or NES is attached to the Cas9-IID protein for optimal expression and nuclear targeting in eukaryotic cells, e.g., human cells.

### Functional Mutations of Cas9-IID Proteins

Various mutations or modifications can be introduced into Cas9-IID proteins as described herein to improve specificity and/or robustness. In some embodiments, the amino acid residues that recognize the protospacer adjacent motif (PAM) are identified. The Cas9-IID proteins described herein can be modified further to recognize different PAMs, e.g., by substituting the amino acid residues that recognize PAM with other amino acid residues.

In some embodiments, the amino acid sequence of the Cas9-IID protein is mutated at one or more amino acid residues to alter its ability to functionally associate with a guide nucleic acid. In some embodiments, the Cas9-IID protein is mutated at one or more amino acid residues to alter its ability to functionally associate with a target nucleic acid. In some embodiments, the Cas9-IID protein described herein are capable of binding to or modifying a target nucleic acid molecule. In some embodiments, the Cas9-IID protein modifies both strands of the target nucleic acid molecule. However, in some embodiments, the Cas9-IID protein is mutated at one or more amino acid residues to alter its nucleic acid manipulation activity. For example, in some embodiments, the Cas9-IID protein can comprise one or more mutations which render the Cas9-IID protein incapable of cleaving a target nucleic acid. In other embodiments, the Cas9-IID protein can comprise one or more mutations such that the Cas9-IID protein is capable of cleaving a single strand of the target nucleic acid (i.e., nickase activity). In some embodiments, the Cas9-IID protein is capable of cleaving the strand of the target nucleic acid that is complementary to the strand to which the guide nucleic acid hybridizes. In some embodiments, the Cas9-IID protein is capable of cleaving the strand of the target nucleic acid to which the guide nucleic acid hybridizes.

In some embodiments, a Cas9-IID protein described herein may be engineered to comprise a deletion in one or more amino acid residues to reduce the size while retaining one or more desired functional activities (e.g., nuclease activity and the ability to interact functionally with a guide nucleic acid). The truncated Cas9-IID protein may be advantageously used in combination with delivery systems having load limitations.

### Functional Domains

The Cas9-IID proteins described herein have nuclease activity. However, they can be modified to have reduced nuclease activity, e.g., nuclease inactivation of at least 50%, at least 60%, at least 70%, at least 80%, at least 90%, at least 95%, at least 97%, or 100% as compared with the wild-type sequence. The nuclease activity can be reduced by, for example, introducing mutations (such as amino acid insertions, deletions, or substitutions) into the nuclease domains of the Cas9-IID proteins described herein. In some embodiments, catalytic residues for the nuclease activities are identified, and these amino acid residues can be substituted by different amino acid residues (e.g., glycine or alanine) to diminish the nuclease activity.

The inactivated Cas9-IID proteins can comprise (e.g., via fusion protein, linker peptides, Gly4Ser (SEQ ID NO: 773) (GS) peptide linkers, etc.) or be associated (e.g., via co-expression of multiple proteins) with one or more functional domains. These functional domains can have various activities, e.g., methylase activity, demethylase activity, transcription activation activity, transcription repression activity, transcription release factor activity, histone modification activity, RNA cleavage activity, DNA cleavage activity, nucleic acid binding activity, and switch activity (e.g., light inducible). Accordingly, in some embodiments of any one of the aspects described herein, the Cas9-IID protein can be associated with a functional domain (also referred to as an effector domain herein), e.g., a domain having transposase activity, methylase activity, demethylase activity, translation activation activity, translation repression activity, transcription activation activity, transcription repression activity, transcription release factor activity, chromatin modifying or remodeling activity, histone modification activity, nuclease activity, single-strand RNA cleavage activity, double-strand RNA cleavage activity, single-strand DNA cleavage activity, double-strand DNA cleavage activity, nucleic acid binding activity, and/or detectable activity. For example, the Cas9-IID protein further comprises a functional domain having transposase activity, methylase activity, demethylase activity, translation activation activity, translation repression activity, transcription activation activity, transcription repression activity, transcription release factor activity, chromatin modifying or remodeling activity, histone modification activity, nuclease activity, single-strand RNA cleavage activity, double-strand RNA cleavage activity, single-strand DNA cleavage activity, double-strand DNA cleavage activity, nucleic acid binding activity, and/or detectable activity.

In some embodiments, the functional domain is a ligase or a functional fragment thereof. In some embodiments, the functional domain is a deaminase or a functional fragment thereof. In some other embodiments, the functional domain is a transposase or a functional fragment thereof. In yet some other embodiments, the functional domain is a reverse transcriptase or a functional fragment thereof. In some embodiments, the functional domain is a transcriptional activation domain or a functional fragment thereof. For example, the functional domain is VP64, VP16, p65, MyoD1, HSF1, RTA, SET7/9, a histone acetyltransferase, or a functional fragment thereof. In some embodiments, the functional domain is a transcription repression domain or a functional fragment thereof. For example, the functional domain is Krüppel associated box (KRAB), SID, or concatemers of SID (e.g., SID4X). In some embodiments, the functional domain is an epigenetic modifying domain or a functional fragment thereof. In some embodiments, the functional domain can be an activation domain or a functional fragment thereof, such as the P65 activation domain.

In some embodiments, the functional domains are selected from the group consisting of Krüppel associated box (KRAB), VP64, VP16, Fok1, P65, HSF1, MyoD1, biotin-APEX, and functional fragments of any one of them.

The functional domain can be operably coupled to the Cas9-IID protein. One or more functional domains can be tethered or linked via a suitable linker (including, but not limited to, GlySer linkers) to the Cas9-IID protein. When there is more than one functional domain, the functional domains can be same or different. In some embodiments, all the functional domains are the same. In some embodiments, all of the functional domains are different from each other. In embodiments, at least two of the functional domains are different from each other. In some embodiments, at least two of the functional domains are the same as each other.

Generally, each functional domain is in a spatial orientation allowing for the functional domain to function in its attributed function. In other words, the positioning of the one or more functional domains on the inactivated Cas9-IID proteins described herein allows for correct spatial orientation for the functional domain to affect the target with the attributed functional effect. For example, if the functional domain is a transcription activator (e.g., VP 16, VP64, or p65), the transcription activator is placed in a spatial orientation that allows it to affect the transcription of the target. Likewise, a transcription repressor (e.g., KRAB) is positioned to affect the transcription of the target, and a nuclease (e.g., Fok1) is positioned to cleave or partially cleave the target. In some embodiments, the functional domain is positioned at the N-terminus of the Cas9-IID proteins described herein. In some embodiments, the functional domain is positioned at the C-terminus of the Cas9-IID proteins described herein. In some embodiments, the inactivated Cas9-IID proteins described herein is modified to comprise a first functional domain at the N-terminus and a second functional domain at the C-terminus.

### Fusion Protein

In another aspect provided herein is a fusion protein comprising a Cas9-IID protein described herein covalently linked to a functional domain. For example, the fusion protein comprises a Cas9-IID protein described herein covalently linked to a functional domain having transposase activity, methylase activity, demethylase activity, translation activation activity, translation repression activity, transcription activation activity, transcription repression activity, transcription release factor activity, chromatin modifying or remodeling activity, histone modification activity, nuclease activity, single-strand RNA cleavage activity, double-strand RNA cleavage activity, single-strand DNA cleavage activity, double-strand DNA cleavage activity, nucleic acid binding activity, and/or detectable activity. It is noted that the functional domain can be linked/fused to the N-terminal Cas9-IID protein. In some other embodiments, the functional domain is linked/fused to the C-terminal Cas9-IID protein. In some embodiments, the functional domain can be linked/fused to the N-terminal or the C-terminal of the Cas9-IID protein via a linker comprising one or more amino acids, i.e., a peptidyl linker.

In some embodiments, the fusion protein comprises a functional domain that is a nucleic acid editing domain.

In some embodiments, the fusion protein comprises a functional domain that is a reverse transcriptase or a functional fragment thereof.

In some embodiments, the fusion protein comprises a functional domain that is a deaminase domain or a functional fragment thereof. The term “deaminase” or “deaminase domain,” as used herein, refers to a protein or enzyme that catalyzes a deamination reaction. In some embodiments, the deaminase or deaminase domain is a naturally-occurring deaminase from an organism, such as a human, chimpanzee, gorilla, monkey, cow, dog, rat, or mouse. In some embodiments, the deaminase or deaminase domain is a variant of a naturally-occurring deaminase from an organism, that does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase from an organism.

In some embodiments, the deaminase or deaminase domain is a cytidine deaminase. A cytidine deaminase domain may also be referred to interchangeably as a cytosine deaminase domain. In some embodiments, the cytidine deaminase catalyzes the hydrolytic deamination of cytidine (C) or deoxycytidine (dC) to uridine (U) or deoxyuridine (dU), respectively. In some embodiments, the cytidine deaminase domain catalyzes the hydrolytic deamination of cytosine (C) to uracil (U). In some embodiments, the cytidine deaminase catalyzes the hydrolytic deamination of cytidine or cytosine in deoxyribonucleic acid (DNA). Without wishing to be bound by any particular theory, fusion proteins comprising a cytidine deaminase are useful inter alia for targeted editing, referred to herein as “base editing,” of nucleic acid sequences in vitro and in vivo.

In some embodiments, the cytidine deaminase or cytidine deaminase domain is a naturally-occurring cytidine deaminase from an organism, such as a human, chimpanzee, gorilla, monkey, cow, dog, rat, or mouse. In some embodiments, the cytidine deaminase or cytidine deaminase domain is a variant of a naturally-occurring cytidine deaminase from an organism that does not occur in nature. For example, in some embodiments, the cytidine deaminase or cytidine deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring cytidine deaminase from an organism, such as a human, chimpanzee, gorilla, monkey, cow, dog, rat, or mouse

In some embodiments, the cytidine deaminase is an apolipoprotein B mRNA-editing complex (APOBEC) family deaminase. In some embodiments, the cytidine deaminase is an APOBEC1 deaminase. In some embodiments, the cytidine deaminase is an APOBEC2 deaminase. In some embodiments, the cytidine deaminase is an APOBEC3 deaminase. In some embodiments, the cytidine deaminase is an APOBEC3A deaminase. In some embodiments, the cytidine deaminase is an APOBEC3B deaminase. In some embodiments, the cytidine deaminase is an APOBEC3C deaminase. In some embodiments, the cytidine deaminase is an APOBEC3D deaminase. In some embodiments, the cytidine deaminase is an APOBEC3E deaminase. In some embodiments, the cytidine deaminase is an APOBEC3F deaminase. In some embodiments, the cytidine deaminase is an APOBEC3G deaminase. In some embodiments, the cytidine deaminase is an APOBEC3H deaminase. In some embodiments, the cytidine deaminase is an APOBEC4 deaminase. In some embodiments, the cytidine deaminase is an activation-induced deaminase (AID). In some embodiments, the cytidine deaminase is a vertebrate cytidine deaminase. In some embodiments, the cytidine deaminase is an invertebrate cytidine deaminase. In some embodiments, the cytidine deaminase is a human, chimpanzee, gorilla, monkey, cow, dog, rat, or mouse deaminase. In some embodiments, the cytidine deaminase is a human cytidine deaminase. In some embodiments, the cytidine deaminase is a rat cytidine deaminase, e.g., rAPOBEC1. In some embodiments, the cytidine deaminase is a Petromyzon marinus cytidine deaminase 1 (pmCDA1). In some embodiments, the cytidine deaminase is a human APOBEC3G. In some embodiments, the cytidine deaminase is a fragment of the human APOBEC3G. In some embodiments, the deaminase is a human APOBEC3G variant comprising a D316R and D317R mutation. In some embodiments, the deaminase is a fragment of the human APOBEC3G and comprising mutations corresponding to the D316R and D317R mutations.

In some embodiments, the cytidine deaminase domain is at least 80%, at least 85%, at least 90%, at least 92%, at least 95%, at least 96%, at least 97%, at least 98%, at least 99%, or at least 99.5% identical to the cytidine deaminase domain of any one of the exemplary cytidines deaminase sequences shown in Table B. In some embodiments, the cytidine deaminase domain comprises the amino acid sequence of any one of the exemplary cytidines deaminase sequences shown in Table B. It should be understood that, in some embodiments, the active domain of the respective sequence can be used, e.g., the domain without a localizing signal (nuclear localization sequence, without nuclear export signal, cytoplasmic localizing signal).

TABLE B
Exemplary cytidine deaminase sequences

| | SEQ ID | |
|---|---|---|
| SEQUENCE | NO | Type/Organism |
| MDSLLMNRRKFLYQFKNVRW AKGRRETYLCYVVKRRDSAT | 45 | AID [Homo |
| SFSLDFGYLRNKNGCHVELL FLRYISDWDLDPGRCYRVTW | | sapiens] |
| FTSWSPCYDCARHVADFLRG | | |
| NPNLSLRIFTARLYFCEDRKAEPEGLRRLHRAGVQIAIMT | | |
| FKDYFYCWNTFVENHERTFK AWEGLHENSVRLSRQLRRIL | | |
| LPLYEVDDLRDAFRTLGL | | |
| MDSLLMKQKK FLYHFKNVRW AKGRHETYLC | 46 | AID [Mus |
| YVVKRRDSAT SCSLDFGHLR NKSGCHVELL FLRYISDWDL | | musculus] |
| DPGRCYRVTW FTSWSPCYDC ARHVAEFLRW NPNLSLRIFT | | |
| ARLYFCEDRKAEPEGLRRLH RAGVQIGIMT FKDYFYCWNT | | |
| FVENRERTFK AWEGLHENSV RLTRQLRRIL LPLYEVDDLR | | |
| DAFRMLGF | | |
| MDSLLMKQRK FLYHFKNVRW AKGRHETYLC YVVKRRDSAT | 47 | AID [Canis |
| SFSLDFGHLR NKSGCHVELL FLRYISDWDL DPGRCYRVTW | | familiaris] |
| FTSWSPCYDC ARHVADFLRG YPNLSLRIFA | | |
| ARLYFCEDRKAEPEGLRRLH RAGVQIAIMT FKDYFYCWNT | | |
| FVENREKTFK AWEGLHENSV RLSRQLRRIL LPLYEVDDLR | | |
| DAFRTLGL | | |
| MDSLLKKQRQ FLYQFKNVRW AKGRHETYLC YVVKRRDSPT | 48 | AID [Bos |
| SFSLDFGHLR NKAGCHVELL FLRYISDWDL DPGRCYRVTW | | taurus] |
| FTSWSPCYDC ARHVADFLRG YPNLSLRIFT | | |
| ARLYFCDKERKAEPEGLRRL HRAGVQIAIM TFKDYFYCWN | | |
| TFVENHERTF KAWEGLHENS VRLSRQLRRI LLPLYEVDDL | | |
| RDAFRTLGL | | |
| MAVGSKPKAA LVGPHWERER IWCFLCSTGL GTQQTGQTSR | 49 | AID [Rattus] |
| WLRPAATQDP VSPPRSLLMK QRKFLYHFKN VRWAKGRHET | | |
| YLCYVVKRRD SATSFSLDFG YLRNKSGCHV | | |
| ELLFLRYISDWDLDPGRCYR VTWFTSWSPC YDCARHVADF | | |
| LRGNPNLSLR IFTARLTGWG ALPAGLMSPA RPSDYFYCWN | | |
| TFVENHERTF KAWEGLHENS VRLSRRLRRI LLPLYEVDDL | | |
| RDAFRTLGL | | |
| MGPFCLGCSH RKCYSPIRNL ISQETFKFHF KNLGYAKGRK | 50 | ApoBEC-3 |
| DTFLCYEVTR KDCDSPVSLH HGVFKNKDNI HAEICFLYWF | | [Mus |
| HDKVLKVLSP REEFKITWYM SWSPCFECAE | | musculus] |
| QIVRFLATHHNLSLDIFSSR LYNVQDPETQ QNLCRLVQEG | | |
| AQVAAMDLYE FKKCWKKFVD NGGRRFRPWKRLLTNFRYQD | | |
| SKLQEILRPC YIPVPSSSSS TLSNICLTKG LPETRFCVEG | | |
| RRMDPLSEEEFYSQFYNQRV KHLCYYHRMK PYLCYQLEQF | | |
| NGQAPLKGCL LSEKGKQHAE ILFLDKIRSMELSQVTITCY | | |
| LTWSPCPNCA WQLAAFKRDR PDLILHIYTS RLYFHWKRPF | | |
| QKGLCSLWQSGILVDVMDLP QFTDCWTNFV NPKRPFWPWK | | |
| GLEIISRRTQ RRLRRIKESW GLQDLVNDFGNLQLGPPMS | | |
| MGPFCLGCSH RKCYSPIRNL ISQETFKFHF KNLRYAIDRK | 51 | APOBEC-3 |
| DTFLCYEVTR KDCDSPVSLHHGVFKNKDNI HAEICFLYWF | | [Rattus] |
| HDKVLKVLSP REEFKITWYM SWSPCFECAE | | |
| QVLRFLATHHNLSLDIFSSR LYNIRDPENQ QNLCRLVQEG | | |
| AQVAAMDLYE FKKCWKKFVD NGGRRFRPWKKLLTNFRYQD | | |
| SKLQEILRPC YIPVPSSSSS TLSNICLTKG LPETRFCVER | | |
| RRVHLLSEEEFYSQFYNQRV KHLCYYHGVK PYLCYQLEQF | | |
| NGQAPLKGCL LSEKGKQHAE ILFLDKIRSMELSQVIITCY | | |
| LTWSPCPNCA WQLAAFKRDR PDLILHIYTS RLYFHWKRPF | | |
| QKGLCSLWQSGILVDVMDLP QFTDCWTNFV NPKRPFWPWK | | |
| GLEIISRRTQ RRLHRIKESW GLQDLVNDFGNLQLGPPMS | | |
| MVEPMDPRTF VSNFNNRPIL SGLNTVWLCC EVKTKDPSGP | 52 | APOBEC-3G |
| PLDAKIFQGK VYSKAKYHPEMRFLRWFHKW RQLHHDQEYK | | [Macaca |
| VTWYVSWSPC TRCANSVATF LAKDPKVTLT | | mulatta] |
| IFVARLYYFWKPDYQQALRI LCQKRGGPHA TMKIMNYNEF | | |
| QDCWNKFVDG RGKPFKPRNN LPKHYTLLQATLGELLRHLM | | |
| DPGTFTSNFN NKPWVSGQHE TYLCYKVERL HNDTWVPLNQ | | |
| HRGFLRNQAPNIHGFPKGRH AELCFLDLIP FWKLDGQQYR | | |
| VTCFTSWSPC FSCAQEMAKF ISNNEHVSLCIFAARIYDDQ | | |
| GRYQEGLRAL HRDGAKIAMM NYSEFEYCWD TFVDRQGRPF | | |
| QPWDGLDEHSQALSGRLRAI | | |
| MKPHFRNPVE RMYQDTFSDN FYNRPILSHR NTVWLCYEVK | 53 | APOBEC-3G |
| TKGPSRPPLD AKIFRGQVYSKLKYHPEMRF FHWFSKWRKL | | [Pan |
| HRDQEYEVTW YISWSPCTKC TRDVATFLAE | | troglodytes] |
| DPKVTLTIFVARLYYFWDPD YQEALRSLCQ KRDGPRATMK | | |
| IMNYDEFQHC WSKFVYSQRE LFEPWNNLPKYYILLHIMLG | | |
| EILRHSMDPP TFTSNFNNEL WVRGRHETYL CYEVERLHND | | |
| TWVLLNQRRGFLCNQAPHKH GFLEGRHAEL CFLDVIPFWK | | |
| LDLHQDYRVT CFTSWSPCFS CAQEMAKFISNNKHVSLCIF | | |
| AARIYDDQGR CQEGLRTLAK AGAKISIMTY SEFKHCWDTF | | |
| VDHQGCPFQPWDGLEEHSQA LSGRLRAILQ NQGN | | |
| MNPQIRNMVE QMEPDIFVYY FNNRPILSGR NTVWLCYEVK | 54 | APOBEC- |
| TKDPSGPPLD ANIFQGKLYPEAKDHPEMKF LHWFRKWRQL | | 3G[Chlorocebus |
| HRDQEYEVTW YVSWSPCTRC ANSVATFLAE | | sabaeus] |
| DPKVTLTIFVARLYYFWKPD YQQALRILCQ ERGGPHATMK | | |
| IMNYNEFQHC WNEFVDGQGK PFKPRKNLPKHYTLLHATLG | | |
| ELLRHVMDPG TFTSNFNNKP WVSGQRETYL CYKVERSHND | | |
| TWVLLNQHRGFLRNQAPDRH GFPKGRHAEL CFLDLIPFWK | | |
| LDDQQYRVTC FTSWSPCFSC AQKMAKFISNNKHVSLCIFA | | |
| ARIYDDQGRC QEGLRTLHRD GAKIAVMNYS EFEYCWDTFV | | |
| DRQGRPFQPWDGLDEHSQAL SGRLRAI | | |
| MKPHFRNTVE RMYRDTFSYN FYNRPILSRR NTVWLCYEVK | 55 | APOBEC-3G |
| TKGPSRPPLD AKIFRGQVYSELKYHPEMRF FHWFSKWRKL | | [Homo sapiens] |
| HRDQEYEVTW YISWSPCTKC TRDMATFLAE | | |
| DPKVTLTIFVARLYYFWDPD YQEALRSLCQ KRDGPRATMK | | |
| IMNYDEFQHC WSKFVYSQRE LFEPWNNLPKYYILLHIMLG | | |
| EILRHSMDPP TFTFNFNNEP WVRGRHETYL CYEVERMHND | | |
| TWVLLNQRRGFLCNQAPHKH GFLEGRHAEL CFLDVIPFWK | | |
| LDLDQDYRVT CFTSWSPCFS CAQEMAKFISKNKHVSLCIF | | |
| TARIYDDQGR CQEGLRTLAE AGAKISIMTY SEFKHCWDTF | | |
| VDHQGCPFQPWDGLDEHSQD LSGRLRAILQ NQEN | | |
| MKPHFRNTVE RMYRDTFSYN FYNRPILSRR NTVWLCYEVK | 56 | APOBEC-3F |
| TKGPSRPRLD AKIFRGQVYSQPEHHAEMCF LSWFCGNQLP | | [Homo sapiens] |
| AYKCFQITWF VSWTPCPDCV AKLAEFLAEH | | |
| PNVTLTISAARLYYYWERDY RRALCRLSQA GARVKIMDDE | | |
| EFAYCWENFV YSEGQPFMPW YKFDDNYAFLHRTLKEILRN | | |
| PMEAMYPHIF YFHFKNLRKA YGRNESWLCF TMEVVKHHSP | | |
| VSWKRGVFRNQVDPETHCHA ERCFLSWFCD DILSPNTNYE | | |
| VTWYTSWSPC PECAGEVAEF LARHSNVNLTIFTARLYYFW | | |
| DTDYQEGLRS LSQEGASVEI MGYKDFKYCW ENFVYNDDEP | | |
| FKPWKGLKYNFLFLDSKLQE ILE | | |
| MNPQIRNPME RMYRDTFYDN FENEPILYGR SYTWLCYEVK | 57 | APOBEC-3B |
| IKRGRSNLLW DTGVFRGQVYFKPQYHAEMC FLSWFCGNQL | | Homo sapiens |
| PAYKCFQITW FVSWTPCPDC VAKLAEFLSE | | |
| HPNVTLTISAARLYYYWERD YRRALCRLSQ AGARVTIMDY | | |
| EEFAYCWENF VYNEGQQFMP WYKFDENYAFLHRTLKEILR | | |
| YLMDPDTFTF NFNNDPLVLR RRQTYLCYEV ERLDNGTWVL | | |
| MDQHMGFLCNEAKNLLCGFY GRHAELRFLD LVPSLQLDPA | | |
| QIYRVTWFIS WSPCFSWGCA GEVRAFLQENTHVRLRIFAA | | |
| RIYDYDPLYK EALQMLRDAG AQVSIMTYDE FEYCWDTFVY | | |
| RQGCPFQPWDGLEEHSQALS GRLRAILQNQ GN | | |
| MQPQGLGPNA GMGPVCLGCS HRRPYSPIRN PLKKLYQQTF | 58 | APOBEC- |
| YFHFKNVRYA WGRKNNFLCYEVNGMDCALP VPLRQGVFRK | | 3B[Rattus] |
| QGHIHAELCF IYWFHDKVLR VLSPMEEFKV | | |
| TWYMSWSPCSKCAEQVARFL AAHRNLSLAI FSSRLYYYLR | | |
| NPNYQQKLCR LIQEGVHVAA MDLPEFKKCWNKFVDNDGQP | | |
| FRPWMRLRIN FSFYDCKLQE IFSRMNLLRE DVFYLQFNNS | | |
| HRVKPVQNRYYRRKSYLCYQ LERANGQEPL KGYLLYKKGE | | |
| QHVEILFLEK MRSMELSQVR ITCYLTWSPCPNCARQLAAF | | |
| KKDHPDLILR IYTSRLYFYW RKKFQKGLCT LWRSGIHVDV | | |
| MDLPQFADCWTNFVNPQRPF RPWNELEKNS WRIQRRLRRI | | |
| KESWGL | | |
| DGWEVAFRSG TVLKAGVLGV SMTEGWAGSG | 59 | APOBEC-3B |
| HPGQGACVWT PGTRNTMNLL REVLFKQQFGNQPRVPAPYY | | [Bos taurus] |
| RRKTYLCYQL KQRNDLTLDR GCFRNKKQRH AEIRFIDKIN | | |
| SLDLNPSQSYKIICYITWSP CPNCANELVN FITRNNHLKL | | |
| EIFASRLYFH WIKSFKMGLQ DLQNAGISVAVMTHTEFEDC | | |
| WEQFVDNQSR PFQPWDKLEQ YSASIRRRLQ RILTAPI | | |
| MNPQIRNPME WMYQRTFYYN FENEPILYGR SYTWLCYEVK | 60 | APOBEC-3B |
| IRRGHSNLLW DTGVFRGQMYSQPEHHAEMC FLSWFCGNQL | | [Pan |
| SAYKCFQITW FVSWTPCPDC VAKLAKFLAE | | troglodytes] |
| HPNVTLTISAARLYYYWERD YRRALCRLSQ AGARVKIMDD | | |
| EEFAYCWENF VYNEGQPFMP WYKFDDNYAFLHRTLKEIIR | | |
| HLMDPDTFTF NFNNDPLVLR RHQTYLCYEV ERLDNGTWVL | | |
| MDQHMGFLCNEAKNLLCGFY GRHAELRFLD LVPSLQLDPA | | |
| QIYRVTWFIS WSPCFSWGCA GQVRAFLQENTHVRLRIFAA | | |
| RIYDYDPLYK EALQMLRDAG AQVSIMTYDE FEYCWDTFVY | | |
| RQGCPFQPWDGLEEHSQALS GRLRAILQVR ASSLCMVPHR | | |
| PPPPPQSPGP CLPLCSEPPL GSLLPTGRPAPSLPFLLTAS | | |
| FSFPPPASLP PLPSLSLSPG HLPVPSFHSL TSCSIQPPCS | | |
| SRIRETEGWASVSKEGRDLG | | |
| MNPQIRNPMK AMYPGTFYFQ FKNLWEANDR NETWLCFTVE | 61 | APOBEC-3C |
| GIKRRSVVSW KTGVFRNQVDSETHCHAERC FLSWFCDDIL | | [Homo sapiens] |
| SPNTKYQVTW YTSWSPCPDC AGEVAEFLAR | | |
| HSNVNLTIFTARLYYFQYPC YQEGLRSLSQ EGVAVEIMDY | | |
| EDFKYCWENF VYNDNEPFKP WKGLKTNFRLLKRRLRESLQ | | |
| MNPQIRNPMK AMYPGTFYFQ FKNLWEANDR NETWLCFTVE | 62 | Gorilla |
| GIKRRSVVSW KTGVFRNQVDSETHCHAERC FLSWFCDDIL | | APOBEC3C |
| SPNTNYQVTW YTSWSPCPEC AGEVAEFLAR | | [Gorilla |
| HSNVNLTIFTARLYYFQDTD YQEGLRSLSQ EGVAVKIMDY | | gorilla] |
| KDFKYCWENF VYNDDEPFKP WKGLKYNFRFLKRRLQEILE | | |
| MEASPASGPR HLMDPHIFTS NFNNGIGRHK TYLCYEVERL | 63 | APOBEC-3A |
| DNGTSVKMDQ HRGFLHNQAKNLLCGFYGRH AELRFLDLVP | | [Homo sapiens] |
| SLQLDPAQIY RVTWFISWSP CFSWGCAGEV | | |
| RAFLQENTHVRLRIFAARIY DYDPLYKEAL QMLRDAGAQV | | |
| SIMTYDEFKH CWDTFVDHQG CPFQPWDGLDEHSQALSGRL | | |
| RAILQNQGN | | |
| MDGSPASRPR HLMDPNTFTF NFNNDLSVRG RHQTYLCYEV | 64 | APOBEC-3A |
| ERLDNGTWVP MDERRGFLCNKAKNVPCGDY GCHVELRFLC | | [Macaca |
| EVPSWQLDPA QTYRVTWFIS WSPCFRRGCA | | mulatta] |
| GQVRVFLQENKHVRLRIFAA RIYDYDPLYQ EALRTLRDAG | | |
| AQVSIMTYEE FKHCWDTFVD RQGRPFQPWDGLDEHSQALS | | |
| GRLRAILQNQ GN | | |
| MDEYTFTENF NNQGWPSKTY LCYEMERLDG DATIPLDEYK | 65 | APOBEC-3A |
| GFVRNKGLDQ PEKPCHAELYFLGKIHSWNL DRNQHYRLTC | | [Bos taurus] |
| FISWSPCYDC AQKLTTFLKE NHHISLHILA | | |
| SRIYTHNRFGCHQSGLCELQ AAGARITIMT FEDFKHCWET | | |
| FVDHKGKPFQ PWEGLNVKSQ ALCTELQAILKTQQN | | |
| MALLTAETFR LQFNNKRRLR RPYYPRKALL CYQLTPQNGS | 66 | APOBEC-3H |
| TPTRGYFENK KKCHAEICFINEIKSMGLDE TQCYQVTCYL | | [Homo sapiens] |
| TWSPCSSCAW ELVDFIKAHD HLNLGIFASR | | |
| LYYHWCKPQQKGLRLLCGSQ VPVEVMGFPK FADCWENFVD | | |
| HEKPLSFNPY KMLEELDKNS RAIKRRLERIKIPGVRAQGR | | |
| YMDILCDAEV | | |
| MALLTAKTFS LQFNNKRRVN KPYYPRKALL CYQLTPQNGS | 67 | APOBEC-3H |
| TPTRGHLKNK KKDHAEIRFINKIKSMGLDE TQCYQVTCYL | | [Macaca |
| TWSPCPSCAG ELVDFIKAHR HLNLRIFASR | | mulatta] |
| LYYHWRPNYQEGLLLLCGSQ VPVEVMGLPE FTDCWENFVD | | |
| HKEPPSFNPS EKLEELDKNS QAIKRRLERIKSRSVDVLEN | | |
| GLRSLQLGPV TPSSSIRNSR | | |
| MNPQIRNPME RMYRDTFYDN FENEPILYGR SYTWLCYEVK | 68 | APOBEC-3D |
| IKRGRSNLLW DTGVFRGPVLPKRQSNHRQE VYFRFENHAE | | [Homo sapiens] |
| MCFLSWFCGN RLPANRRFQI TWFVSWNPCL | | |
| PCVVKVTKFLAEHPNVTLTI SAARLYYYRD RDWRWVLLRL | | |
| HKAGARVKIM DYEDFAYCWE NFVCNEGQPFMPWYKFDDNY | | |
| ASLHRTLKEI LRNPMEAMYP HIFYFHFKNL LKACGRNESW | | |
| LCFTMEVTKHHSAVFRKRGV FRNQVDPETH CHAERCFLSW | | |
| FCDDILSPNT NYEVTWYTSW SPCPECAGEVAEFLARHSNV | | |
| NLTIFTARLC YFWDTDYQEG LCSLSQEGAS VKIMGYKDFV | | |
| SCWKNFVYSDDEPFKPWKGL QTNFRLLKRR LREILQ | | |
| MTSEKGPSTG DPTLRRRIEP WEFDVFYDPR ELRKEACLLY | 69 | APOBEC-1 |
| EIKWGMSRKI WRSSGKNTTNHVEVNFIKKF TSERDFHPSM | | [Homo sapiens] |
| SCSITWFLSW SPCWECSQAI REFLSRHPGV | | |
| TLVIYVARLFWHMDQQNRQG LRDLVNSGVT IQIMRASEYY | | |
| HCWRNFVNYP PGDEAHWPQY PPLWMMLY ALELHCIILSLP | | |
| PCLKISRRWQ NHLTFFRLHL QNCHYQTIPP HILLATGLIH | | |
| PSVAWR | | |
| MSSETGPVAV DPTLRRRIEP HEFEVFFDPR ELRKETCLLY | 70 | APOBEC-1 |
| EINWGGRHSV WRHTSQNTSNHVEVNFLEKF TTERYFRPNT | | [Mus |
| RCSITWFLSW SPCGECSRAI TEFLSRHPYV | | musculus] |
| TLFIYIARLYHHTDQRNRQG LRDLISSGVT IQIMTEQEYC | | |
| YCWRNFVNYP PSNEAYWPRY PHLWVKLYVLELYCIILGLP | | |
| PCLKILRRKQ PQLTFFTITL QTCHYQRIPP HLLWATGLK | | |
| MSSETGPVAV DPTLRRRIEP HEFEVFFDPR ELRKETCLLY | 71 | APOBEC-1 |
| EINWGGRHSI WRHTSQNTNKHVEVNFIEKF TTERYFCPNT | | [Rattus] |
| RCSITWFLSW SPCGECSRAI TEFLSRYPHV | | |
| TLFIYIARLYHHADPRNRQG LRDLISSGVT IQIMTEQESG | | |
| YCWRNFVNYS PSNEAHWPRY PHLWVRLYVLELYCIILGLP | | |
| PCLNILRRKQ PQLTFFTIAL QSCHYQRLPP HILWATGLK | | |
| MAQKEEAAVA TEAASQNGED LENLDDPEKL KELIELPPFE | 72 | APOBEC-2 |
| IVTGERLPAN FFKFQFRNVEYSSGRNKTFL CYVVEAQGKG | | [Homo sapiens] |
| GQVQASRGYL EDEHAAAHAE EAFFNTILPA | | |
| FDPALRYNVTWYVSSSPCAA CADRIIKTLS KTKNLRLLIL | | |
| VGRLFMWEEP EIQAALKKLK EAGCKLRIMKPQDFEYVWQN | | |
| FVEQEEGESK AFQPWEDIQE NFLYYEEKLA DILK | | |
| MAQKEEAAEA AAPASQNGDD LENLEDPEKL KELIDLPPFE | 73 | APOBEC-2 |
| IVTGVRLPVN FFKFQFRNVEYSSGRNKTFL CYVVEVQSKG | | [Mus |
| GQAQATQGYL EDEHAGAHAE EAFFNTILPA | | musculus] |
| FDPALKYNVTWYVSSSPCAA CADRILKTLS KTKNLRLLIL | | |
| VSRLFMWEEP EVQAALKKLK EAGCKLRIMKPQDFEYIWQN | | |
| FVEQEEGESK AFEPWEDIQE NFLYYEEKLA DILK | | |
| MAQKEEAAEA AAPASQNGDD LENLEDPEKL KELIDLPPFE | 74 | APOBEC-2 |
| IVTGVRLPVN FFKFQFRNVEYSSGRNKTFL CYVVEAQSKG | | [Rattus] |
| GQVQATQGYL EDEHAGAHAE EAFFNTILPA | | |
| FDPALKYNVTWYVSSSPCAA CADRILKTLS KTKNLRLLIL | | |
| VSRLFMWEEP EVQAALKKLK EAGCKLRIMKPQDFEYLWQN | | |
| FVEQEEGESK AFEPWEDIQE NFLYYEEKLA DILK | | |
| MAQKEEAAAA AEPASQNGEE VENLEDPEKL KELIELPPFE | 75 | APOBEC-2 |
| IVTGERLPAH YFKFQFRNVEYSSGRNKTFL CYVVEAQSKG | | [Bos taurus] |
| GQVQASRGYL EDEHATNHAE EAFFNSIMPT | | |
| FDPALRYMVTWYVSSSPCAA CADRIVKTLN KTKNLRLLIL | | |
| VGRLFMWEEP EIQAALRKLK EAGCRLRIMKPQDFEYIWQN | | |
| FVEQEEGESK AFEPWEDIQE NFLYYEEKLA DILK | | |
| MAQKEEAAAA AEPASQNGEE VENLEDPEKL KELIELPPFE | 76 | CDA1 |
| IVTGERLPAH YFKFQFRNVEYSSGRNKTFL CYVVEAQSKG | | [Petromyzon |
| GQVQASRGYL EDEHATNHAE EAFFNSIMPT | | marinus] |
| FDPALRYMVTWYVSSSPCAA CADRIVKTLN KTKNLRLLIL | | |
| VGRLFMWEEP EIQAALRKLK EAGCRLRIMKPQDFEYIWQN | | |
| FVEQEEGESK AFEPWEDIQE NFLYYEEKLA DILK | | |
| MKPHFRNTVE RMYRDTFSYN FYNRPILSRR NTVWLCYEVK | 77 | APOBEC3GD |
| TKGPSRPPLD AKIFRGQVYSELKYHPEMRF FHWFSKWRKL | | 316R_D317R[ |
| HRDQEYEVTW YISWSPCTKC TRDMATFLAE | | Homo sapiens] |
| DPKVTLTIFVARLYYFWDPD YQEALRSLCQ KRDGPRATMK | | |
| IMNYDEFQHC WSKFVYSQRE LFEPWNNLPKYYILLHIMLG | | |
| EILRHSMDPP TFTFNFNNEP WVRGRHETYL CYEVERMHND | | |
| TWVLLNQRRGFLCNQAPHKH GFLEGRHAEL CFLDVIPFWK | | |
| LDLDQDYRVT CFTSWSPCFS CAQEMAKFISKNKHVSLCIF | | |
| TARIYRRQGR CQEGLRTLAE AGAKISIMTY SEFKHCWDTF | | |
| VDHQGCPFQPWDGLDEHSQD LSGRLRAILQ NQEN | | |
| MDPPTFTFNF NNEPWVRGRH ETYLCYEVER MHNDTWVLLN | 78 | APOBEC3G |
| QRRGFLCNQA PHKHGFLEGRHAELCFLDVI PFWKLDLDQD | | [Homo sapiens] |
| YRVTCFTSWS PCFSCAQEMA KFISKNKHVS | | |
| LCIFTARIYDDQGRCQEGLR TLAEAGAKIS IMTYSEFKHC | | |
| WDTFVDHQGC PFQPWDGLDE HSQDLSGRLRAILQ | | |
| MDPPTFTFNF NNEPWVRGRH ETYLCYEVER MHNDTWVLLN | 79 | APOBEC3GD |
| QRRGFLCNQA PHKHGFLEGRHAELCFLDVI PFWKLDLDQD | | 120R D121R |
| YRVTCFTSWS PCFSCAQEMA KFISKNKHVS | | [Homo sapiens] |
| LCIFTARIYRRQGRCQEGLR TLAEAGAKIS IMTYSEFKHC | | |
| WDTFVDHQGC PFQPWDGLDE HSQDLSGRLRAILQ | | |

In another example, the fusion protein comprises a functional domain that is an adenosine deaminase domain or a functional fragment thereof. In some embodiments, the deaminase or deaminase domain is an adenosine deaminase, which catalyzes the hydrolytic deamination of adenine or adenosine. In some embodiments, the deaminase or deaminase domain is an adenosine deaminase, catalyzing the hydrolytic deamination of adenosine or deoxyadenosine to inosine or deoxyinosine, respectively. In some embodiments, the adenosine deaminase catalyzes the hydrolytic deamination of adenine or adenosine in deoxyribonucleic acid (DNA). The adenosine deaminases (e.g., engineered adenosine deaminases, evolved adenosine deaminases) provided herein may be from any organism, such as a bacterium. In some embodiments, the deaminase or deaminase domain is a variant of a naturally-occurring deaminase from an organism. In some embodiments, the deaminase or deaminase domain does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase. In some embodiments, the adenosine deaminase is from a bacterium, such as E. coli, S. aureus, S. typhi, S. putrefaciens, H. influenzae, or C. crescentus. In some embodiments, the adenosine deaminase is a TadA deaminase. In some embodiments, the TadA deaminase is an E. coli TadA deaminase (ecTadA). In some embodiments, the TadA deaminase is a truncated E. coli TadA deaminase. For example, the truncated ecTadA may be missing one or more N-terminal amino acids relative to a full-length ecTadA. In some embodiments, the truncated ecTadA may be missing 1, 2, 3, 4, 5, 6, 7, 8, 9, 10, 11, 12, 13, 14, 15, 6, 17, 18, 19, or 20 N-terminal amino acid residues relative to the full length ecTadA. In some embodiments, the truncated ecTadA may be missing 1, 2, 3, 4, 5, 6, 7, 8, 9, 10, 11, 12, 13, 14, 15, 6, 17, 18, 19, or 20 C-terminal amino acid residues relative to the full length ecTadA. In some embodiments, the ecTadA deaminase does not comprise an N-terminal methionine.

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any one of the exemplary adenosine deaminase sequences shown in Table C, or to any of the adenosine deaminases provided herein. It should be appreciated that adenosine deaminases provided herein may include one or more mutations (e.g., any of the mutations provided herein). In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to any one of the amino acid sequences set forth in SEQ ID NOs: 80-100, or any of the adenosine deaminases provided herein. In some embodiments, the adenosine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170 identical contiguous amino acid residues as compared to any one of the amino acid sequences set forth in any one of the exemplary adenosine deaminase sequences shown in Table C, or any of the adenosine deaminases provided herein.

TABLE C
Exemplary adenosine deaminase sequences

| | SEQ ID | |
|---|---|---|
| SEQUENCE | NO | Type/organism |
| EACKALGTWR LEGATLYVTL EPCPMCAGAV VLSRVEKVVF | 80 | TadA |
| GAFDPKGGCS GTLMNLLQEERFNHQAEVVS GVLEEECGGM | | [Bacillus |
| LSAFFRELRK KKKAARKNLS E | | subtilis |
| MTQDELYMKE AIKEAKKAEE KGEVPIGAVL VINGEIIARA | 81 | TadA |
| HNLRETEQRS IAHAEMLVIDEACKALGTWR LEGATLYVTL | | [Bacillus |
| EPCPMCAGAV VLSRVEKVVF GAFDPKGGCS | | subtilis] |
| GTLMNLLQEERFNHQAEVVS GVLEEECGGM LSAFFRELRK | | |
| KKKAARKNLS E | | |
| MSEVEFSHEY WMRHALTLAK RAWDEREVPV GAVLVHNNRV | 82 | TadA. |
| IGEGWNRPIG RHDPTAHAAIMALRQGGLVM QNYRLIDATL | | catalytically |
| YVTLEPCVMC AGAMIHSRIG RVVFGARDAK | | inactive |
| TGAAGSLMDVLHHPGMNHRV EITEGILADE CAALLSDFFR | | (E59A) [E. |
| MRRQEIKAQK KAQSSTD | | coli] |
| MSEVEFSHEY WMRHALTLAK RARDEREVPV GAVLVLNNRV | 83 | TadA 7.10 |
| IGEGWNRAIG LHDPTAHAEIMALRQGGLVM QNYRLIDATL | | [Synthetic] |
| YVTFEPCVMC AGAMIHSRIG RVVFGVRNAK | | |
| TGAAGSLMDVLHYPGMNHRV EITEGILADE CAALLCYFFR | | |
| MPRQVFNAQK KAQSSTD | | |
| MSEVEFSHEY WMRHALTLAK RARDEREVPV GAVLVLNNRV | 84 | TadA |
| IGEGWNRAIG LHDPTAHAEIMALRQGGLVM QNYRLIDATL | | 7.10(V106W) |
| YVTFEPCVMC AGAMIHSRIG RVVFGWRNAK | | [Synthetic] |
| TGAAGSLMDVLHYPGMNHRV EITEGILADE CAALLCYFFR | | |
| MPRQVFNAQK KAQSSTD | | |
| MSEVEFSHEY WMRHALTLAK RARDEREVPV GAVLVLNNRV | 85 | TadA |
| IGEGWNRAIG LHDPTAHAEIMALRQGGLVM QNYRLIDATL | | 7.10(N108W) |
| YVTFEPCVMC AGAMIHSRIG RVVFGVRWAK | | [Synthetic] |
| TGAAGSLMDVLHYPGMNHRV EITEGILADE CAALLCYFFR | | |
| MPRQVFNAQK KAQSSTD | | |
| MSEVEFSHEY WMRHALTLAK RARDEREVPV GAVLVLNNRV | 86 | TadA |
| IGEGWNRAIG LHDPTAHAEIMALRQGGLVM QNYRLIDATL | | 7.10(N108Q) |
| YVTFEPCVMC AGAMIHSRIG RVVFGVRQAK | | [Synthetic] |
| TGAAGSLMDVLHYPGMNHRV EITEGILADE CAALLCYFFR | | |
| MPRQVFNAQK KAQSSTD | | |
| MSEVEFSHEY WMRHALTLAK RARDEREVPV GAVLVLNNRV | 87 | TadA |
| IGEGWNRAIG LHDPTAHAEIMALRQGGLVM QNYRLIDATL | | 7.10(V106F) |
| YVTFEPCVMC AGAMIHSRIG RVVFGFRNAK | | [Synthetic] |
| TGAAGSLMDVLHYPGMNHRV EITEGILADE CAALLCYFFR | | |
| MPRQVFNAQK KAQSSTD | | |
| MSEVEFSHEY WMRHALTLAK RARDEREVPV GAVLVLNNRV | 88 | tadA 7.10 |
| IGEGWNRAIG LHDPTAHAEIMALRQGGLVM QNYRLIDATL | | (V106Q) |
| YVTFEPCVMC AGAMIHSRIG RVVFGQRNAK | | [Synthetic] |
| TGAAGSLMDVLHYPGMNHRV EITEGILADE CAALLCYFFR | | |
| MPRQVFNAQK KAQSSTD | | |
| MSEVEFSHEY WMRHALTLAK RARDEREVPV GAVLVLNNRV | 89 | tadA 7.10 |
| IGEGWNRAIG LHDPTAHAEIMALRQGGLVM QNYRLIDATL | | (V106M) |
| YVTFEPCVMC AGAMIHSRIG RVVFGMRNAK | | [Synthetic] |
| TGAAGSLMDVLHYPGMNHRV EITEGILADE CAALLCYFFR | | |
| MPRQVFNAQK KAQSSTD | | |
| MSEVEFSHEY WMRHALTLAK RARDEREVPV GAVLVLNNRV | 90 | tadA |
| IGEGWNFAIG LHDPTAHAEIMALRQGGLVM QNYRLIDATL | | 7.10(R47F) |
| YVTFEPCVMC AGAMIHSRIG RVVFGVRNAK | | [Synthetic] |
| TGAAGSLMDVLHYPGMNHRV EITEGILADE CAALLCYFFR | | |
| MPRQVFNAQK KAQSSTD | | |
| MSEVEFSHEY WMRHALTLAK RARDEREVPV GAVLVLNNRV | 91 | tadA |
| IGEGWNWAIG LHDPTAHAEIMALRQGGLVM QNYRLIDATL | | 7.10(R47W) |
| YVTFEPCVMC AGAMIHSRIG RVVFGVRNAK | | [Synthetic] |
| TGAAGSLMDVLHYPGMNHRV EITEGILADE CAALLCYFFR | | |
| MPRQVFNAQK KAQSSTD | | |
| MSEVEFSHEY WMRHALTLAK RARDEREVPV GAVLVLNNRV | 92 | tadA |
| IGEGWNQAIG LHDPTAHAEIMALRQGGLVM QNYRLIDATL | | 7.10(R47Q) |
| YVTFEPCVMC AGAMIHSRIG RVVFGVRNAK | | [Synthetic] |
| TGAAGSLMDVLHYPGMNHRV EITEGILADE CAALLCYFFR | | |
| MPRQVFNAQK KAQSSTD | | |
| MSEVEFSHEY WMRHALTLAK RARDEREVPV GAVLVLNNRV | 93 | tadA |
| IGEGWNMAIG LHDPTAHAEIMALRQGGLVM QNYRLIDATL | | 7.10(R47M) |
| YVTFEPCVMC AGAMIHSRIG RVVFGVRNAK | | [Synthetic] |
| TGAAGSLMDVLHYPGMNHRV EITEGILADE CAALLCYFFR | | |
| MPRQVFNAQK KAQSSTD | | |
| MSEVEFSHEY WMRHALTLAK RAWDEREVPV GAVLVHNNRV | 94 | TadA(E59Q) |
| IGEGWNRPIG RHDPTAHAQIMALRQGGLVM QNYRLIDATL | | [Synthetic] |
| YVTLEPCVMC AGAMIHSRIG RVVFGARDAK | | |
| TGAAGSLMDVLHHPGMNHRV EITEGILADE CAALLSDFFR | | |
| MRRQEIKAQK KAQSSTD | | |
| MSEVEFSHEY WMRHALTLAK RAWDEREVPV GAVLVHNNRV | 95 | TadA, N- |
| IGEGWNRPIG RHDPTAHAEIMALRQGGLVM QNYRLIDATL | | terminal |
| YVTLEPCVMC AGAMIHSRIG RVVFGARDAK | | truncated |
| TGAAGSLMDVLHHPGMNHRV EITEGILADE CAALLSDFFR | | [E. coli] |
| MRRQEIKAQK KAQSSTD | | |
| MRRAFITGVF FLSEVEFSHE YWMRHALTLA KRAWDEREVP | 96 | TadA [E. |
| VGAVLVHNNR VIGEGWNRPIGRHDPTAHAE IMALRQGGLV | | coli] |
| MQNYRLIDAT LYVTLEPCVM CAGAMIHSRI | | |
| GRVVFGARDAKTGAAGSLMD VLHHPGMNHR VEITEGILAD | | |
| ECAALLSDFF RMRRQEIKAQ KKAQSSTD | | |
| MGSHMTNDIY FMTLAIEEAK KAAQLGEVPI GAIITKDDEV | 97 | TadA |
| IARAHNLRET LQQPTAHAEHIAIERAAKVL GSWRLEGCTL | | [Staphylococcus |
| YVTLEPCVMC AGTIVMSRIP RVVYGADDPK | | aureus] |
| GGCSGSLMNLLQQSNFNHRA IVDKGVLKEA CSTLLTTFFK | | |
| NLRANKKSTN | | |
| MPPAFITGVT SLSDVELDHE YWMRHALTLA KRAWDEREVP | 98 | TadA [S. |
| VGAVLVHNHR VIGEGWNRPIGRHDPTAHAE IMALRQGGLV | | typhimurium] |
| LQNYRLLDTT LYVTLEPCVM CAGAMVHSRI | | |
| GRVVFGARDAKTGAAGSLID VLHHPGMNHR VEIIEGVLRD | | |
| ECATLLSDFF RMRRQEIKAL KKADRAEGAGPAV | | |
| MDEYWMQVAM QMAEKAEAAG EVPVGAVLVK DGQQIATGYN | 99 | TadA [S. |
| LSISQHDPTA HAEILCLRSAGKKLENYRLL DATLYITLEP | | putrefaciens] |
| CAMCAGAMVH SRIARVVYGA RDEKTGAAGT | | |
| VVNLLQHPAFNHQVEVTSGV LAEACSAQLS RFFKRRRDEK | | |
| KALKLAQRAQ QGIE | | |
| MDAAKVRSEF DEKMMRYALE LADKAEALGE IPVGAVLVDD | 100 | TadA [H. |
| ARNIIGEGWN LSIVQSDPTAHAEIIALRNG AKNIQNYRLL | | influenzae] |
| NSTLYVTLEP CTMCAGAILH SRIKRL VFGA | | |
| SDYKTGAIGSRFHFFDDYKM NHTLEITSGV LAEECSQKLS | | |
| TFFQKRREEK KIEKALLKSL SDK | | |

In some cases, the adenosine deaminase is double-stranded RNA-specific adenosine deaminase (ADAR). Examples of ADARs include those described Yiannis A Savva et al., The ADAR protein family, Genome Biol. 2012; 13(12): 252, which is incorporated by reference in its entirety. In some examples, the ADAR may be hADAR1. In certain examples, the ADAR may be hADAR2. The sequence of hADAR2 may be that described under Accession No. AF525422.1.

In some cases, the deaminase may be a deaminase domain, e.g., a deaminase domain of ADAR (“ADAR-D”). In one example, the deaminase may be the deaminase domain of hADAR2 (“hADAR2-D), e.g., as described in Phelps K J et al., Recognition of duplex RNA by the deaminase domain of the RNA editing enzyme ADAR2. Nucleic Acids Res. 2015 January; 43(2): 1123-32, which is incorporated by reference herein in its entirety. In a particular example, the hADAR2-D has a sequence comprising amino acid 299-701 of hADAR2-D, e.g., amino acid 299-701 of the sequence under Accession No. AF525422.1. In certain examples, the adenosine deaminase may comprise one or more of the mutations: E488Q based on amino acid sequence positions of hADAR2-D, and mutations in a homologous ADAR protein corresponding to the above. In one embodiment, the adenosine deaminase may comprise one or more of the mutations: E488Q, V351G, based on amino acid sequence positions of hADAR2-D, and mutations in a homologous ADAR protein corresponding to the above. In one embodiment, the adenosine deaminase may comprise one or more of the mutations: E488Q, V351G, S486A, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, I398V, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, I398V, K350I, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, I398V, K350I, M383L, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, I398V, K350I, M383L, D619G, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, I398V, K350I, M383L, D619G, S582T, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, I398V, K350I, M383L, D619G, S582T, V440I based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, I398V, K350I, M383L, D619G, S582T, V440I, S495N based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, I398V, K350I, M383L, D619G, S582T, V440I, S495N, K418E based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, I398V, K350I, M383L, D619G, S582T, V440I, S495N, K418E, S661T based on amino acid sequence positions of hADAR2-D, and mutations in a homologous ADAR protein corresponding to the above. In some examples, provided herein includes a mutated adenosine deaminase e.g., an adenosine deaminase comprising one or more mutations of E488Q, V351G, S486A, T375S, S370C, P462A, N597I, L332I, I398V, K350I, M383L, D619G, S582T, V440I, S495N, K418E, S661T. In some examples, provided herein includes a mutated adenosine deaminase e.g., an adenosine deaminase comprising E488Q, V351G, S486A, T375S, S370C, P462A, N597I, L332I, I398V, K350I, M383L, D619G, S582T, V440I, S495N, K418E, and S661T. In some examples, provided herein includes a mutated adenosine deaminase e.g., an adenosine deaminase comprising E488Q, V351G, S486A, T375S, S370C, P462A, N597I, L332I, I398V, K350I, M383L, D619G, S582T, V440I, S495N, K418E, S661T, and S375N. In one embodiment, the adenosine deaminase may be a tRNA-specific adenosine deaminase or a variant thereof. In one embodiment, the adenosine deaminase may comprise one or more of the mutations: W23L, W23R, R26G, H36L, N37S, P48S, P48T, P48A, I49V, R51L, N72D, L84F, S97C, A106V, D108N, H123Y, G125A, A142N, S146C, D147Y, R152H, R152P, E155V, I156F, K157N, K161T, based on amino acid sequence positions of E. coli TadA, and mutations in a homologous deaminase protein corresponding to the above. In one embodiment, the adenosine deaminase may comprise one or more of the mutations: D108N based on amino acid sequence positions of E. coli TadA, and mutations in a homologous deaminase protein corresponding to the above. In one embodiment, the adenosine deaminase may comprise one or more of the mutations: A106V, D108N, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, based on amino acid sequence positions of E. coli TadA, and mutations in a homologous deaminase protein corresponding to the above. In one embodiment, the adenosine deaminase may comprise one or more of the mutations: A106V, D108N, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, A142N, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, H36L, R51L, S146C, K157N, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, H36L, R51L, S146C, K157N, P48S, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, H36L, R51L, S146C, K157N, P48S, A142N, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, H36L, R51L, S146C, K157N, P48S, W23R, P48A, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, H36L, R51L, S146C, K157N, P48S, W23R, P48A, A142N, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, H36L, R51L, S146C, K157N, P48S, W23R, P48A, R152P, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, H36L, R51L, S146C, K157N, P48S, W23R, P48A, R152P, A142N, based on amino acid sequence positions of E. coli TadA, and mutations in a homologous deaminase protein corresponding to the above.

In some embodiments, the fusion protein further comprises a second adenosine deaminase domain. The second adenosine deaminase can be an ecTadA domain, a variant, or a functional fragment thereof. In some embodiments, the first and second adenosine deaminase domain independently comprises a nucleotide sequence having at least 80%, at least 85%, at least 90%, at least 92%, at least 95%, at least 96%, at least 97%, at least 98%, at least 99%, at least 99.5%, or 100% identity to any one of to any one of the sequences in Table C. In one example, both the first and second adenosine deaminase domains are an ecTadA domain, a variant, or a functional fragment thereof.

In some embodiments, the fusion protein comprises a functional domain that is uracil glycosylase inhibitor (UGI) domain or a functional fragment thereof.

The term “uracil glycosylase inhibitor” or “UGI,” as used herein, refers to a protein that is capable of inhibiting a uracil-DNA glycosylase base-excision repair enzyme. In some embodiments, a UGI domain comprises a wild-type UGI or a UGI as set forth in Table D. In some embodiments, the UGI proteins provided herein include fragments of UGI and proteins homologous to a UGI or a UGI fragment. For example, in some embodiments, a UGI domain comprises a fragment of the amino acid sequence set forth in Table D. In some embodiments, a UGI fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or at least 99.5% of the amino acid sequence as set forth in TABLE D. In some embodiments, a UGI comprises an amino acid sequence homologous to the amino acid sequence set forth in TABLE D, or an amino acid sequence homologous to a fragment of the amino acid sequence set forth in TABLE D. In some embodiments, proteins comprising UGI or fragments of UGI or homologs of UGI or UGI fragments are referred to as “UGI variants.” A UGI variant shares homology to UGI, or a fragment thereof. For example, a UGI variant is at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to a wild type UGI or a UGI as set forth in Table D. In some embodiments, the UGI variant comprises a fragment of UGI, such that the fragment is at least 70% identical, at least 80% identical, at least 90% identical, at least 95% identical, at least 96% identical, at least 97% identical, at least 98% identical, at least 99% identical, at least 99.5% identical, or at least 99.9% to the corresponding fragment of wild-type UGI or a UGI as set forth in Table D.

TABLE D
Exemplary UGI sequences

| | SEQ ID | |
|---|---|---|
| SEQUENCE | NO | Type/organism |
| MTNLSDIIEK ETGKQLVIQE SILMLPEEVE EVIGNKPESD | 101 | UNGI_BPPB2[Bacillus |
| ILVHTAYDES TDENVMLLTSDAPEYKPWAL | | phagePBS2 |
| VIQDSNGENK IKML | | |
| KGHLTRLGLE FFDQPAVPLA RAFLGQVLVR | 102 | Truncated AAG |
| RLPNGTELRG RIVETQAYLG | | nuclease (E125Q) |
| PEDEAAHSRGGRQTPRNRGM FMKPGTLYVY | | [H. sapiens] |
| IIYGMYFCMN ISSQGDGACV LLRALEPLEG | | |
| LETMRQLRSTLRKGTASRVL KDRELCSGPS | | |
| KLCQALAINK SFDQRDLAQD | | |
| EAVWLERGPLEPSEPAVVAAARVGVGHAGEWARKPLRF | | |
| YVRGSPWVSVVD RVAEQDTQA | | |
| DLASLRAQQI ELASSVIRED RLDKDPPDLI | 103 | Endo V |
| AGAAVGFEQG GEVTRAAMVL LKYPSLELVE | | nuclease(D35A) |
| YKVARIATTM PYIPGFLSFR EYPALLAAWE | | [Synthetic] |
| MLSQKPDLVF VDGHGISHPR | | |
| RLGVASHFGLLVDVPTIGVA KKRLCGKFEP | | |
| LSSEPGALAP LMDKGEQLAW VWRSKARCNP | | |
| LFIATGHRVS VDSALAWVQR CMKGYRLPEP | | |
| TRWADAVASE RPAFVRYTANQP | | |
| MASRGVNKVILVGNLGQDPEVRYMPNGGAVANITLATS | 104 | SSB [Erwinia |
| ESWRDKQTGETKEKTEWHRVVLFGKLAEVAGEYLRKGS | | tasmaniensis] |
| QVYIEGALQTRKWTDQAGVEKYTTEVVVNVGGTMQML | | |
| GGRSQGGGASAGGONGGSNNGWGQPQQPQGGNQFSGG | | |
| AQQQARPQQQPQONNAPANNEPPIDFDDDIP | | |
| MAGAQDFVPHTADLAELAAAAGECRGCGLYRDATQAV | 105 | UdgX [Synthetic] |
| FGAGGRSARIMMIGEQPGDKEDLAGLPFVGPAGRLLDRA | | |
| LEAADIDRDALYVTNAVKHFKFTRAAGGKRRIHKTPSRT | | |
| EVVACRPWLIAEMTSVEPDVVVLLGATAAKALLGNDFR | | |
| VTQHRGEVLHVDDVPGDPALVATVHPSSLLRGPKEERES | | |
| AFAGLVDDLRVAADVRP | | |
| MIGQKTLYSFFSPSPARKRHAPSPEPAVQGTGVAGVPEES | 106 | UDG [Synthetic] |
| GDAAAIPAKKAPAGQEEPGTPPSSPLSAEQLDRIQRNKAA | | |
| ALLRLAARNVPVGFGESWKKHLSGEFGKPYFIKLMGFVA | | |
| EERKHYTVYPPPHQVFTWTQMCDIKDVKVVILGQEPYH | | |
| GPNQAHGLCFSVQRPVPPPPSLENIYKELSTDIEDFVHPGH | | |
| GDLSGWAKQGVLLLNAVLTVRAHQANSHKERGWEQFT | | |
| DAVVSWLNQNSNGLVFLLWGSYAQKKGSAIDRKRHHV | | |
| LQTAHPSPLSVYRGFFGCRHFSKTNELLQKSGKKPIDWK | | |
| EL | | |

In yet another example, the fusion protein comprises a functional domain that is reverse transcriptase domain or a functional fragment thereof. A reverse transcriptase (RT) is an enzyme used to generate complementary DNA (cDNA) from an RNA template, a process termed reverse transcription. Reverse transcriptases are used by retroviruses to replicate their genomes, by retrotransposon mobile genetic elements to proliferate within the host genome, by eukaryotic cells to extend the telomeres at the ends of their linear chromosomes, and by some non-retroviruses such as the hepatitis B virus, a member of the Hepadnaviridae, which are dsDNA-RT viruses. Retroviral RT has three sequential biochemical activities: RNA-dependent DNA polymerase activity, ribonuclease H, and DNA-dependent DNA polymerase activity. Collectively, these activities enable the enzyme to convert single-stranded RNA into double-stranded cDNA. In an embodiment, the RT domain of a reverse transcriptase is used in the present invention. The domain may include only the RNA-dependent DNA polymerase activity. In some examples, the RT domain is non-mutagenic, i.e., does not cause mutation in the donor polynucleotide (e.g., during the reverse transcriptase process). In some examples, the RT domain may be non-retron RT, e.g., a viral RT or human endogenous RTs. In some examples, the RT domain may be retron RT or DGRs RT. In some example, the RT may be less mutagenic than a counterpart wildtype RT. In one embodiment, the RT herein is not mutagenic. Exemplary the reverse transcriptase include, but are not limited to, Human immunodeficiency virus (HIV) RT, Avian myeloblastosis virus (AMV) RT, Moloney murine leukemia virus (M-MLV) RT a group II intron RT, a group II intron-like RT, or a chimeric RT. In some embodiments, the functional domain comprises modified forms of these RTs, such as, engineered variants of Avian myeloblastosis virus (AMV) RT, Moloney murine leukemia virus (M-MLV) RT, or Human immunodeficiency virus (HIV) RT (see, e.g., Anzalone, et al., Search-and-replace genome editing without double-strand breaks or donor DNA, Nature. 2019 December; 576(7785): 149-157). Without wishing to be bound by any particular theory, fusion proteins comprising a reverse transcriptase domain are useful inter alia for targeted editing, referred to herein as “prime editing,” of nucleic acid sequences in vitro and in vivo.

In some embodiments, a DNA and RNA targeting complex described herein produces fewer indels in a target sequence that does not comprise the canonical PAM at is 3′-end as compared to the number of indels produced by a complex comprising a wild-type Cas9-IID and wild-type gRNA. For example, a DNA and RNA targeting complex described herein produces at least 2-fold, at least 3-fold, at least 4-fold, at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold fewer indels in a target sequence that does not comprise the canonical PAM at is 3′-end as compared to the number of indels produced by a complex comprising a wild-type Cas9-IID and wild-type gRNA. In some embodiments, indels can be measured using high-throughput sequencing.

In some embodiments, a DNA and RNA targeting complex described herein, where the Cas9-IID protein comprises a deaminase domain, exhibits an increased deamination efficacy in a target sequence that does not comprise the canonical PAM at is 3′-end as compared to the deamination activity of a complex comprising a wild-type Cas9-IID and wild-type gRNA. For example, the deamination efficiency of a DNA and RNA targeting complex, where the Cas9-IID protein comprises a deaminase domain, in a target sequence having a 3′-end that is not directly adjacent to the sequence is at least is at least 2-fold, at least 3-fold, at least 4-fold, at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold higher as compared to the deamination efficiency of a complex comprising a wild-type Cas9-IID and wild-type gRNA. The deamination activity can be measured using a deamination assay, PCR, sequencing or any combination thereof.

### Guide Nucleic Acids

As used herein, a “guide nucleic acid”, “gNA” refers to a nucleic acid that facilitates the targeting of Cas9-IID protein described herein to a target nucleic acid. A guide nucleic acid can be RNA, DNA or a mix of RNA/DNA. Guide nucleic acids are also referred to as guide RNA or gRNA herein. Generally, the guide nucleic acid comprises a guide sequence, also referred to as a spacer or spacer sequence herein. The guide sequence can be referred to as a “cr sequence” herein. The guide nucleic acid can further include a tracr sequence for complexing with the protein effector polypeptide. The term “tracr sequence”, as used herein, can generally refer to a nucleic acid with at least about 5%, 10%, 20%, 30%, 40%, 50%, 60%, 70%, 80%, 90%, 95%, or 100% sequence identity and/or sequence similarity to a wild type exemplary tracrRNA sequence. In some embodiments of any one of the aspects described herein, the tracr sequence comprises one or more, e.g., 1, 2, 3, 4 or more hairpins or stem loops. In some embodiments, the tracr sequence comprises a sequence predicted to comprise at least two hairpins comprising less than 5 base-paired ribonucleotides. The spacer and the tracr sequences can be linked to each other via a hairpin or stem loop structure. The tracr sequence can be 5 or more nucleotides in length. For example, the tracr sequence can be 6, 7, 8, 9, 10, 11, 12, 13, 14, 15, 16, 17, 18, 19, 20, 25, 30, 40, 50, or more nucleotides in length. It is noted that when the spacer and the tracr sequence can be located in any preferred position to each other. For example, the spacer can be located 5′ of the tracr sequence. Alternatively, the spacer can be located 3′ of the tracr sequence. In some preferred embodiments, the spacer is 5′ of the tracr sequence.

The ability of a guide nucleic acid to direct sequence-specific binding of a Cas9-IID protein described herein a target nucleic acid sequence can be assessed by any suitable assay. For example, components sufficient to form a complex comprising a Cas9-IID protein and the guide nucleic acid to be tested, can be provided to a host cell having the corresponding target nucleic acid sequence, such as by transfection with vectors encoding the components of the complex, followed by an assessment of preferential targeting (e.g., cleavage) within the target nucleic acid sequence, such as by Surveyor assay described in WO2022087494, content of which is incorporated herein by reference. Similarly, cleavage of a target nucleic acid sequence (or a sequence in the vicinity thereof) can be evaluated in a test tube by providing the target nucleic acid sequence, and components sufficient to form a complex comprising a Cas9-IID protein and the guide nucleic acid to be tested, and comparing binding or rate of cleavage at or in the vicinity of the target sequence between the test and control guide nucleic acid reactions. Other assays are possible, and will occur to those skilled in the art.

Some exemplary gRNAs amenable to various aspects described herein are described, for example, in PCT publications WO2022087494, WO2020180699, WO2021226363, WO2022261292, WO2021119275, WO2019237069, WO2018107028, WO2019067872 and WO2019067992, and PCT Application No. PCT/US2024/022237, content of each of which is incorporated herein by reference in their entireties.

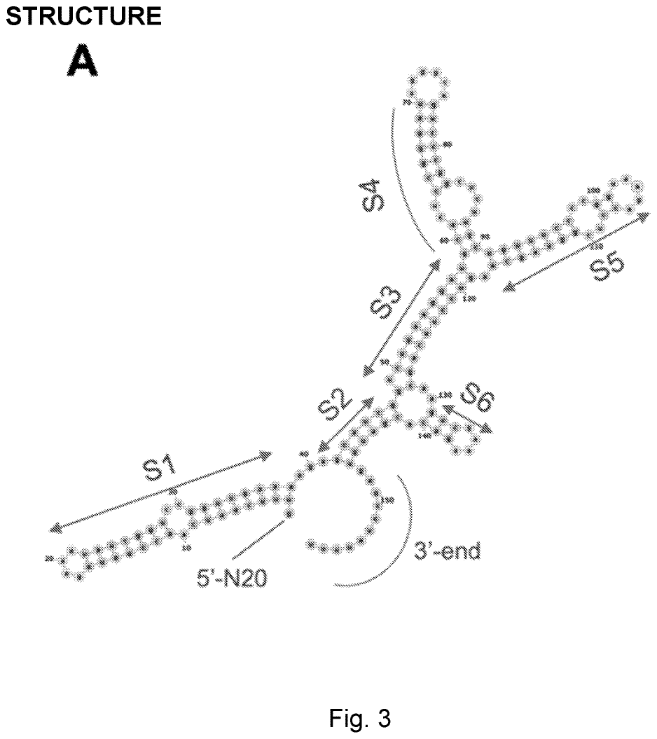

In some embodiments, the guide RNA has a secondary structure as shown in FIG. 3, i.e., the sequence of the guide RNA forms a secondary structure similar to the secondary structure shown in FIG. 3. Thus, in some embodiments, the guide RNA comprises, in series, a 5′-N region, S1′ region, a S1″ region substantially complementary to the s1′ region, a S2′ region, a S3′ region, a S4′ region, a S4″ region substantially complementary to the S4′ region, a S5′ region, a S5″ region substantially complementary to the S5′ region, a D3″ region substantially complementary to the S3′ region, a S6′ region, a S6″ region substantially complementary to the S6′ region, a S2″ region substantially complementary to the S2′ region, and 3′-tail region. It is noted that each region is connected to the next region by 1 or more (e.g., 1, 2, 3, 4, 5, 6, 7, 8, 9, 10 or more) nucleotides, or directly by a modified (e.g., phosphorothioate, imidp, or MMI) or unmodified (e.g., phosphodiester) internucleoside linkage. Each region can be independently from 1 to 25 (e.g., 2, 3, 4, 5, 6, 7, 8, 9, 10, 11, 12, 13, 14, 15, 16, 17, 18, 19, 20, 21, 22, 23, 24 or 25) nucleotides in length.

In some embodiments, one or more complementary regions can be absent. For example, the S5′ and the S5″ regions can be absent. In another non-limiting example, the S6′ and S6″ regions can be absent. In yet another non-limiting example, the S5′, S5″, S6′ and S6″ regions can be absent. It is noted that the absent regions can be replaced with a linker, e.g., a single-stranded region (i.e a pin-loop). In some embodiments, the S5′, S5″, S6′ and S6″ regions are absent, and the duplex formed by S3′ and S3″ regions does not comprise a bulge loop, e.g., the nucleotide (i.e., U) forming the bulge loop in Structure A is absent/deleted.

The 5′-N region is absent, or 1 or more (e.g., 2, 3, 4, 5, 6, 7, 8, 9, 10, or more nucleotides in length. In some embodiments, the 5′-N region comprises is 1, 2, 3, 4 or 5 nucleotides in length. For example, the 5′-N region is 1, 2, or 3, preferably 1 or 2, more preferably 1 nucleotide in length.

In some embodiments, the S1′ and S1″ regions are independently 5 to 30 nucleotides in length. For example, the S2′ and S1″ independently are 5 to 25 (e.g., 5, 6, 7, 8, 9, 10, 11, 12, 13, 14, 15, 16, 17, 18, 19, 20, 21, 22, 23, 24 or 25, preferably 6, 7, 8, 9, 10, 11, 12, 13, 14, 15, or 16, more preferably 7, 8, 9, 10, 11, 12, 13, 14 or 15) nucleotides in length. In some embodiments, the S1′ and S2″ region together form a double stranded structure (duplex region), optionally the duplex comprises an internal loop comprising 2 to 10 (e.g., 2, 3, 4, 5, 6, 7, 8 or 9) nucleotides, and/or a bulge loop comprising 1 to 10 (e.g., 2, 3, 4, 5, 6, 7, 8 or 9) nucleotides. Preferably, the duplex comprises an internal loop comprising 2 to 10 (e.g., 2, 3, 4, 5, 6, 7, 8 or 9) nucleotides.

In some embodiments, the S2′ and S2″ regions are independently 2 to 10 (e.g., 2, 3, 4, 5, 6, 7, 8, 9, or 10, preferably 3, 4, 5, 6, 7, 8 or 9, more preferably 5, 6, or 7) nucleotides in length. In some embodiments, the S2′ and S2″ region together form a double stranded structure), optionally the duplex comprises an internal loop comprising 2 to 10 (e.g., 2, 3, 4, 5, 6, 7, 8 or 9) nucleotides, and/or a bulge loop comprising 1 to 10 (e.g., 2, 3, 4, 5, 6, 7, 8 or 9) nucleotides. Preferably, the duplex does not comprise an bulge or internal loop.

In some embodiments, the S3′ and S3″ regions are independently are 5 to 25 (e.g., 5, 6, 7, 8, 9, 10, 11, 12, 13, 14, 15, 16, 17, 18, 19, 20, 21, 22, 23, 24 or 25, preferably 8, 9, 10, 11, 12, 13, 14, 15, or 16, more preferably 10, 11, 12, 13, 14 or 15) nucleotides in length. In some embodiments, the S3′ and S3″ region together form a double stranded structure), optionally the duplex comprises an internal loop comprising 2 to 10 (e.g., 2, 3, 4, 5, 6, 7, 8 or 9) nucleotides, and/or a bulge loop comprising 1 to 10 (e.g., 2, 3, 4, 5, 6, 7, 8 or 9) nucleotides. In some embodiments, the duplex region comprises a 1 nucleotide bulge. Preferably, the duplex does not comprise a bulge or internal loop. For example, the nucleotide forming the bulge, i.e., U in Structure A is absent.

In some embodiments, the S4′ and S4″ regions are independently are 5 to 25 (e.g., 5, 6, 7, 8, 9, 10, 11, 12, 13, 14, 15, 16, 17, 18, 19, 20, 21, 22, 23, 24 or 25, preferably 8, 9, 10, 11, 12, 13, 14, 15, or 16, more preferably 10, 11, 12, 13, 14 or 15) nucleotides in length. In some embodiments, the S4′ and S4″ region together form a double stranded structure), optionally the duplex comprises an internal loop comprising 2 to 10 (e.g., 2, 3, 4, 5, 6, 7, 8 or 9) nucleotides, and/or a bulge loop comprising 1 to 10 (e.g., 2, 3, 4, 5, 6, 7, 8 or 9) nucleotides. Preferably, the duplex comprises an internal loop comprising 2 to 10 (e.g., 2, 3, 4, 5, 6, 7, 8 or 9) nucleotides.

In some embodiments, the S5′ and S5″ regions are independently are 5 to 25 (e.g., 5, 6, 7, 8, 9, 10, 11, 12, 13, 14, 15, 16, 17, 18, 19, 20, 21, 22, 23, 24 or 25, preferably 8, 9, 10, 11, 12, 13, 14, 15, or 16, more preferably 10, 11, 12, 13, 14 or 15) nucleotides in length. In some embodiments, the S5′ and S5″ region together form a double stranded structure), optionally the duplex comprises an internal loop comprising 2 to 10 (e.g., 2, 3, 4, 5, 6, 7, 8 or 9) nucleotides, and/or a bulge loop comprising 1 to 10 (e.g., 2, 3, 4, 5, 6, 7, 8 or 9) nucleotides. Preferably, the duplex comprises an internal loop comprising 2 to 10 (e.g., 2, 3, 4, 5, 6, 7, 8 or 9) nucleotides.

In some embodiments, the S6′ and S6″ regions are independently 2 to 10 (e.g., 2, 3, 4, 5, 6, 7, 8, 9, or 10, preferably 2, 3, 4, or 5, more preferably 2, 3, or 4) nucleotides in length. In some embodiments, the S6′ and S6″ region together form a double stranded structure.

The 3′-tail region can be at absent or 1, 2, 3, 4, 5 or more nucleotide in length. For example, the 3′-region can be from about 5 to about 35 nucleotides in length. In some embodiments, the 3′-region comprises 5, 6, 7, 8, 9, 10, 11, 12, 13, 14, 15, 16, 17, 18, 19, 20, 21, 22, 23, 24, 25, 26, 27, 28, 29 or 30 nucleotides. Generally, the 3′-tail region is single-stranded.

The 5′-N regions and the S1′ region can be connected to each other via 1, 2, 3, 4, 5, 6, 7, 8, or more nucleotides, or directly by a modified (e.g., phosphorothioate, midophosphoramidate (imidp), or methylenemethylimino (MMI, 3′-CH₂—N(CH₃)—O-5′) or unmodified (e.g., phosphodiester) internucleoside linkage. For example, the 5′-N and the S1′ regions are connected to each other directly by a modified (e.g., phosphorothioate, imidp, or MMI) or unmodified (e.g., phosphodiester) internucleoside linkage.

The S1′ and S1″ regions can be connected to each other via 1, 2, 3, 4, 5, 6, 7, 8, or more nucleotides, or directly by a modified (e.g., phosphorothioate, imidp, or MMI) or unmodified (e.g., phosphodiester) internucleoside linkage. For example, the S1′ and S2″ regions are connected to each other via 2, 3, 4, 5, 6, 7 or 8, (e.g., 2, 3, 4, 5, or 6, preferably 3, 4, or 5, more preferably 4) nucleotides. In some embodiments, the S1′ and S1″ are connected by a nucleotide sequence comprising GAAA or GAAAA.

The S1″ and S2′ regions can be connected to each other via 1, 2, 3, 4, 5, 6, 7, 8, or more nucleotides, or directly by a modified (e.g., phosphorothioate, imidp, or MMI) or unmodified (e.g., phosphodiester) internucleoside linkage. For example, the S1″ and S2′ regions are connected to each other via 2, 3, 4, 5, 6, 7 or 8, (e.g., 2, 3, 4, 5, or 6, preferably 2, 3, or 4, more preferably 3) nucleotides.

The S2′ and the S3′ regions can be connected to each other via 1, 2, 3, 4, 5, 6, 7, 8, or more nucleotides, or directly by a modified (e.g., phosphorothioate, imidp, or MMI) or unmodified (e.g., phosphodiester) internucleoside linkage. For example, the S2′ and S3′ regions are connected to each other via 1, 2, 3, 4, or 5 (e.g., 1, 2, or 3, preferably 1 or 2, more preferably 3) nucleotides. In some preferred embodiments, the S2′ and S3′ regions are connected directly by a modified (e.g., phosphorothioate, imidp, or MMI) or unmodified (e.g., phosphodiester) internucleoside linkage.

The S3′ and the S4′ regions can be connected to each other via 1, 2, 3, 4, 5, 6, 7, 8, or more nucleotides, or directly by a modified (e.g., phosphorothioate, imidp, or MMI) or unmodified (e.g., phosphodiester) internucleoside linkage. For example, the S3′ and S4′ regions are connected to each other via 1, 2, 3, 4, or 5 (e.g., 1, 2, or 3, preferably 1 or 2, more preferably 3) nucleotides. In some preferred embodiments, the S3′ and S4′ regions are connected directly by a modified (e.g., phosphorothioate, imidp, or MMI) or unmodified (e.g., phosphodiester) internucleoside linkage.

In some embodiments, the S4′ and S4″ regions are connected to each other via 1, 2, 3, 4, 5, 6, 7, or more nucleotides, or directly by a modified (e.g., phosphorothioate, imidp, or MMI) or unmodified (e.g., phosphodiester) internucleoside linkage. For example, the S4′ and S4″ regions are connected to each other by 3, 4, 5, 6, 7, 8, or 9 (e.g., 4, 5, 6, 7, or 8, preferably 5, 6, or 7, more preferably 6) nucleotides.

The S4″ and the S5′ regions can be connected to each other via 1, 2, 3, 4, 5, 6, 7, 8, or more nucleotides, or directly by a modified (e.g., phosphorothioate, imidp, or MMI) or unmodified (e.g., phosphodiester) internucleoside linkage. For example, the S4″ and S5′ regions are connected to each other via 1, 2, 3, 4, or 5 (e.g., 1, 2, or 3, preferably 1 or 2, more preferably 3) nucleotides. In some preferred embodiments, the S4″ and S5′ regions are connected directly by a modified (e.g., phosphorothioate, imidp, or MMI) or unmodified (e.g., phosphodiester) internucleoside linkage.

In some embodiments, the S5′ and S5″ regions are connected to each other via 1, 2, 3, 4, 5, 6, 7, or more nucleotides, or directly by a modified (e.g., phosphorothioate, imidp, or MMI) or unmodified (e.g., phosphodiester) internucleoside linkage. For example, the S5′ ad S5″ regions are connected to each other by 3, 4, 5, 6, 7, 8, or 9 (e.g., 4, 5, 6, 7, or 8, preferably 4, 5, 6, or 7, more preferably 5) nucleotides. In some embodiments, 2 or more (e.g., 3, 4, or 5) contiguous nucleotides connecting the S5′ and S5″ regions are complementary to at least part of a sequence of the 3′-tail region.

The S5″ and the S3″ regions can be connected to each other via 1, 2, 3, 4, 5, 6, 7, 8, or more nucleotides, or directly by a modified (e.g., phosphorothioate, imidp, or MMI) or unmodified (e.g., phosphodiester) internucleoside linkage. For example, the S5″ and S3″ regions are connected to each other via 1, 2, 3, 4, 5, or 6 (e.g., 1, 2, 3, or 4, preferably 1, 2, or 3, more preferably 1) nucleotides.

The S3″ and the S6′ regions can be connected to each other via 1, 2, 3, 4, 5, 6, 7, 8, or more nucleotides, or directly by a modified (e.g., phosphorothioate, imidp, or MMI) or unmodified (e.g., phosphodiester) internucleoside linkage. For example, the S3″ and S6′ regions are connected to each other via 1, 2, 3, 4, 5, or 6 (e.g., 1, 2, 3, or 4, preferably 1, 2, or 3, more preferably 2) nucleotides.

The S6′ and S6″ regions can be connected to each other by 1, 2, 3, 4, 5, 6, 7, 8, or more nucleotides, or directly by a modified (e.g., phosphorothioate, imidp, or MMI) or unmodified (e.g., phosphodiester) internucleoside linkage. For example, the S6′ and S6″ regions are connected to each other via 2, 3, 4, 5, 6, 7 or 8, (e.g., 2, 3, 4, 5, or 6, preferably 3, 4, or 5, more preferably 4) nucleotides.

The S6″ and the S2″ regions can be connected to each other via 1, 2, 3, 4, 5, 6, 7, 8, or more nucleotides, or directly by a modified (e.g., phosphorothioate, imidp, or MMI) or unmodified (e.g., phosphodiester) internucleoside linkage. For example, the S6″ and S2″ regions are connected to each other via 1, 2, 3, 4, 5, or 6 (e.g., 1, 2, 3, or 4, preferably, 1, 2, or 3, more preferably 1) nucleotides.

The S2″ and the 3′-tail regions can be connected to each other via 1, 2, 3, 4, 5, 6, 7, 8, or more nucleotides, or directly by a modified (e.g., phosphorothioate, imidp, or MMI) or unmodified (e.g., phosphodiester) internucleoside linkage. For example, S2″ and the 3′-tail regions are connected to each other via 1, 2, 3, 4, or 5 (e.g., 1, 2, or 3, preferably 1 or 2, more preferably 3) nucleotides. In some preferred embodiments, the S2″ and the 3′-tail regions are connected directly by a modified (e.g., phosphorothioate, imidp, or MMI) or unmodified (e.g., phosphodiester) internucleoside linkage.

In some embodiments, the guide RNA is less than 155 nucleotides long. For example, the guide RNA is less than 154, 153, 152, 151, 150, 149, 148, 147, 146, 145, 144, 143, 142, 141, 140, 139, 138, 137, 136, 135, 134, 133, 132, 131, 130, 129, 128, 127, 126, 125, 124, 123, 122, 121, 120, 119, 118, 117, 116, 115, 114, 113, 112, 111, 110, 109, 108, 107, 106, 105, 104, 103, 102, 101, or 100 nucleotides in length.

In some embodiments, the guide RNA comprises a nucleotide sequence having at least 60%, 65%, 70%, 75%, 80%, 85%, 90%, 95% identity to a nucleotide selected from the group consisting of SEQ ID NOs: 14-26, 28, 315-506, 509-526 or 529-542. For example, the guide RNA comprises a nucleotide sequence having at 100% identity to any one of SEQ ID NOs: 14-26, 28, 315-506, 509-526 or 529-542.

In some embodiments, the guide RNA comprises at least one nucleic acid modification. Exemplary nucleic acid modifications include, but are not limited to, nucleobase modifications (e.g., a non-natural or modified nucleobase), sugar modifications, 5inter-sugar linkage modifications (e.g., modified internucleotide linkages), conjugates (e.g., ligands), and any combinations thereof. Nucleic acid modifications also include unnatural, or degenerate nucleobases. For example, the guide RNA comprises a modified nucleotide selected from the group consisting of 2′-O-methyl (2′-OMe) nucleotides, 2′-fluoro (2′-F) nucleotides, bridged nucleic acid (BNA) nucleotides (e.g., 2′-0,4′-C-methylene (locked nucleic acid, LNA) nucleotides, 2′-0,4′-C-ethylene (locked nucleic acid, ENA) nucleotides, 5′-methyl-BNA, cEt BNA, cMOE BNA, oxy amino BNA and vinyl-carbo BNA), anhydrohexitol (1,5-anhydrohexitol nucleic acid, HNA) nucleotides, cyclohexene (Cyclohexene nucleic acid, CeNA) nucleotides, 2′-methoxyethyl (2′-MOE) nucleotides, 2′-O-allyl nucleotides, 2′-C-allyl ribose nucleotides, 2′-O—N-methylacetamido (2′-O-NMA) nucleotides, a 2′-O-dimethylaminoethoxyethyl (2′-O-DMAEOE) nucleotides, 2′-O-aminopropyl (2′-O-AP) nucleotides, 2′-F arabinose (2′-ara-F) nucleotides, threose (Threose nucleic acid, TNA) nucleotides, and acyclic nucleotides (e.g., unlocked nucleic acids (UNA) and 2,3-dihydroxylpropyl (glycol nucleic acid, GNA)); a modified internucleoside linkage; a non-natural or modified nucleobase; or a combination thereof, preferably, the modified nucleotide is a 2′-O-methyl (2′-OMe) nucleotide or a 2′-fluoro nucleotide, and/or a nucleotide comprising a non-natural or modified nucleobase.

In some embodiments, the guide RNA comprises at least one (e.g., 1, 2, 3, 4, 5, or more) modified internucleoside linkage (e.g., a modified internucleoside linkage selected independently from the group consisting of phosphorothioates (R, S, or racemic), phosphorodithioates, methylenemethylimino (MMI, 3′-CH₂—N(CH₃)—O-5′), phosphotriesters, alkylphosphonates (e.g., methylphosphonates), phosphoramidate, methylenemethylimino (—CH₂—N(CH₃)—O—CH₂—), thiodiester (—O—C(O)—S—), thionocarbamate (—O—C(O)(NH)—S—), siloxane (—O—Si(H)₂—O— and dialkylsiloxane), N,N′-dimethylhydrazine (—CH₂—N(CH₃)—N(CH₃)—), amide-3 (3′-CH₂—C(═O)—N(H)-5′), amide-4 (3′-CH₂—N(H)—C(═O)-5′)), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methykenecarbonylamino, methylenehydrazo, methylenedimethylhydrazo, methyleneoxymethylimino, ethers (C3′-O—C5′), thioethers (C3′-S—C5′), thioacetamido (C3′-N(H)—C(═O)—CH₂—S—C5′, C3′-O—P(O)—O—SS—C5′), C3′-CH₂—NH—NH—C5′, 3′—NHP(O)(OCH₃)—O-5′, 3′-NHP(O)(OCH₃)—O-5′), 2′->5′ internucleoside linkages, 2′->3′ internucleoside linkages, 3′->3′ internucleoside linkages, and 5′->5′ internucleoside linkages, optionally the modified internucleoside linkage is phosphorothioate, imidp or MMI, more preferably the modified internucleoside linkage is phosphorothioate (PS)

In some embodiments, at least one double-stranded structure in the guide RNA comprises a duplex stabilizing modification. Exemplary duplex stabilizing modifications include, but are 2′-F nucleotides, 2′-OMe nucleotides, 2′-methoxyethyl nucleotides, 2,6-diaminopurine nucleotides, 5-methyl cytidine, N4-ethyl cytidine, 5-propynyl cytidine, 5-propynyl uridine, 5-hydroxybutynl-2′-deoxyuridine, 8-aza-7-deazaguanosine, a locked nucleic acid (LNA), and/or covalent cross-linking of two strands of the duplex.

In some embodiments, the 3′-tail region comprises any one of: (i) a modification of any one or more of the last 7, 6, 5, 4, 3, 2, or 1 nucleotides; (ii) one modified nucleotide; (ii) two modified nucleotides; (iii) three modified nucleotides; (iv) four modified nucleotides; (v) five modified nucleotides; (vi) six modified nucleotides; or (vii) seven modified nucleotides.

In some embodiments, the 3′-tail region comprises: (i) a modified internucleoside linkage (e.g., PS, imidp or MMI linkage) between nucleotides; (ii) a 2′-OMe nucleotide; (iii) a 2′-O-MOE nucleotide; (iv) a 2′-F nucleotide; (v) a LNA nucleotide; (vi) a 3′->3′ linkage between nucleotides; (vii) an inverted abasic nucleotide; or (viii) a combination of one or more of (i)-(vii).

In some embodiments, the 3′-tail region comprises: (i) a modified internucleoside linkage (e.g., PS, imidp or MMI linkage) between the last and second to last nucleotides; (ii) a modified internucleoside linkage (e.g., PS, imidp or MMI linkage) between the last and second to last nucleotide and a PS linkage between the second and third to last nucleotides; (iii) modified internucleoside linkages (e.g., PS and/or MMI linkage) between any one or more of the last four nucleotides; (iv) modified internucleoside linkages (e.g., PS and/or MMI linkage) between any one or more of the last five nucleotides; and/or or (v) modified internucleoside linkages (e.g., PS and/or MMI linkage) between any one or more of the last 2, 3, 4, 5, 6, 7, 8, 9, or 10 nucleotides.

In some embodiments, the 3′-tail region comprises: (i) a modification of one or more of the last 1-7 nucleotides, wherein the modification is a modified internucleoside linkages (e.g., PS and/or MMI linkage), inverted abasic nucleotide, a 3′->3′ internucleoside linkage, 2′-OMe, 2′-O-MOE, 2′-F, LNA, or a combination thereof; (ii) a modification to the last nucleotide with 2′-OMe, 2′-O-MOE, 2′-F, LNA, or combinations thereof, and an optional one or two modified internucleoside linkages (e.g., PS and/or MMI linkage) to the next nucleotide; (iii) a modification to the last and/or second to last nucleotide with 2′-OMe, 2′-O-MOE, 2′-F, LNA, or combinations thereof, and optionally one or more modified internucleoside linkages (e.g., PS and/or MMI linkage); iv. a modification to the last, second to last, and/or third to last nucleotides with 2′-OMe, 2′-O-moe, 2′-F, or combinations thereof, and optionally one or more modified internucleoside linkages (e.g., PS and/or MMI linkage); (v) a modification to the last, second to last, third to last, and/or fourth to last nucleotides with 2′-OMe, 2′-O-MOE, 2′-F, LNA or combinations thereof, and optionally one or more modified internucleoside linkages (e.g., PS and/or MMI linkage); and/or (vi) a modification to the last, second to last, third to last, fourth to last, and/or fifth to last nucleotides with 2′-OMe, 2′-O-MOE, 2′-F, LNA, or combinations thereof, and optionally one or more modified internucleoside linkages (e.g., PS and/or MMI linkage).

In some embodiments, the 3′-tail region comprises: (i) a 2′-OMe modified nucleotide at the last position, three consecutive 2′-O-MOE modified nucleotides immediately 5′ to the 2′-OMe modified nucleotide, and three consecutive PS linkages between the last three nucleotides; (ii) five consecutive 2′-OMe modified nucleotides from the 3′ end of the 3′ terminus, and three PS linkages between the last three nucleotides; (iii) an inverted abasic modified nucleotide at the last position; (iv) an inverted abasic modified nucleotide at the last position, and three consecutive 2′-OMe modified nucleotides at the last three positions; (v) 15 consecutive 2′-OMe modified nucleotides from the 3′ end, five consecutive 2′-F modified nucleotides immediately 5′ to the 2′-OMe modified nucleotides, and three PS linkages between the last three nucleotides; (vi) alternating 2′-OMe modified nucleotides and 2′-F modified nucleotides at the last 20 nucleotides, and three PS linkages between the last three nucleotides; (vii) two or three consecutive 2′-OMe modified nucleotides, and three PS linkages between the last three nucleotides; (viii) one PS linkage between the last and next to last nucleotides; and/or (ix) 15 or 20 consecutive 2′-OMe modified nucleotides, and three PS linkages between the last three nucleotides.

In some embodiments, the guide RNA comprises, at its 5′-end, any one of: (i) one modified nucleotide; (ii) two modified nucleotides; (iii) three modified nucleotides; (iv) four modified nucleotides; (v) five modified nucleotides; (vi) six modified nucleotides; or (vii) seven modified nucleotides. For example, the guide RNA comprises, at its 5′-end, between 1 and 7, between 1 and 5, between 1 and 4, between 1 and 3, or between 1 and 2 modified nucleotides.

In some embodiments, the guide RNA comprises, at its 5′-end, one or more of: (i) a modified internucleoside linkage (e.g., a phosphorothioate and/or MMI linkage) between nucleotides; (ii) a 2′-OMe nucleotide; (iii) a 2′-O-MOE nucleotide; (iv) a 2′-F nucleotide; (v) a LNA; (vi) a 3′->3′ linkage; (vii) an inverted abasic modified nucleotide; (viii) a deoxyribonucleotide; (ix) an inosine; and (x) combinations of one or more of (i)-(ix).

In some embodiments, the guide RNA comprises, at its 5′-end, about 1-2, 1-3, 1-4, 1-5, 1-6, or 1-7 modified internucleoside (e.g., a phosphorothioate and/or MMI) linkages between nucleotides. For example, the guide RNA comprises, at its 5′-end, 1, 2, 3, 4, 5, 6, and/or 7 modified internucleoside (e.g., a phosphorothioate and/or MMI) linkages between nucleotides. In some embodiments, the guide RNA comprises, at its 5-end, any one of: (i) one modified internucleoside (e.g., a phosphorothioate and/or MMI) linkage, and the linkage is between nucleotides 1 and 2; (ii) two modified internucleoside (e.g., a phosphorothioate and/or MMI) linkages, and the linkages are between nucleotides 1 and 2, and 2 and 3; (iii) modified internucleoside (e.g., a phosphorothioate and/or MMI) linkages between any one or more of nucleotides 1 and 2, 2 and 3, and 3 and 4; (iv) modified internucleoside (e.g., a phosphorothioate and/or MMI) linkages between any one or more of nucleotides 1 and 2, 2 and 3, 3 and 4, and 4 and 5; (v) modified internucleoside (e.g., a phosphorothioate and/or MMI) linkages between any one or more of nucleotides 1 and 2, 2 and 3, 3 and 4, 4 and 5, and 5 and 6; (vi) modified internucleoside (e.g., a phosphorothioate and/or MMI) linkages between any one or more of nucleotides 1 and 2, 2 and 3, 3 and 4, 4 and 5, 5 and 6, and 6 and 7; or (vii) modified internucleoside (e.g., a phosphorothioate and/or MMI) linkages between any one or more of nucleotides 1 and 2, 2 and 3, 3 and 4, 4 and 5, 5 and 6, 6 and 7, and 7 and 8.

In some embodiments, the guide RNA comprises, at its 5-end, at least one 2′-OMe, 2′-O-MOE, inverted abasic, or 2′-F modified nucleotide.

In some embodiments, gRNA comprises a guide sequence, also referred to as a spacer, spacer sequence, or variable region herein. In some embodiments, the variable region comprises a nucleotide sequence that is complementary to a portion of a target nucleic acid. The variable region can be referred to as a “cr sequence” herein. The nucleotide sequence of the variable region (i.e., guide sequence) can be chosen to direct site-specific binding to a target sequence of a target nucleic acid. The variable region can comprise an engineered heterologous sequence. Generally, the variable region is from about 10 to about 50 nucleotides in length. In some embodiments, the variable region is at least 15 nucleotides in length. For example, the variable region is at least 16 nucleotides, at least 17 nucleotides, at least 18 nucleotides, at least 19 nucleotides, at least 20 nucleotides, at least 21 nucleotides, or at least 22 nucleotides in length. In some embodiments, the variable region is from about 15 to about 17 nucleotides, from about 18 to about 20 nucleotides, from about 21 to about 23 nucleotides, from about 24 to about 26 nucleotides, from about 27 to about 29 nucleotides, from about 30 to about 32 nucleotides, from about 33 to about 35 nucleotides, from about 36 to about 38 nucleotides, from about 39 to about 41 nucleotides, from about 42 to about 44 nucleotides, from about 45 to about 47 nucleotides, or from about 48 to about 50 nucleotides in length. In some embodiments, the spacer is from 15 to 17 nucleotides, from 15 to 23 nucleotides, from 15 to 30 nucleotides, from 16 to 22 nucleotides, from 17 to 20 nucleotides, from 20 to 24 nucleotides (e.g., 20, 21, 22, 23, or 24 nucleotides), from 23 to 25 nucleotides (e.g., 23, 24, or 25 nucleotides), from 24 to 27 nucleotides, from 27 to 30 nucleotides, from 30 to 45 nucleotides (e.g., 30, 31, 32, 33, 34, 35, 40, or 45 nucleotides), from 30 or 35 to 40 nucleotides, from 41 to 45 nucleotides, from 45 to 50 nucleotides in length. In some embodiments, the variable region is 15, 16, 17, 18, 19, 20, 21, 22, 23, 24, 25, 26, 27, 28, 29, 30, 31, 32, 33, 34, 35, 36, 37, 38, 39 40, 41, 42, 43, 44, 45, 46, 47 48, 49, or 50 nucleotides in length. In some preferred embodiments, the variable region is from 16 to 23 nucleotides (e.g., 16, 17, 18, 19, 21, 22 or 23 nucleotides) in length. For example, the variable region is 19, 20 or 21 nucleotides in length.

In some embodiments, the variable region or guide sequence comprises from about 10 to about 100 nucleotides and a sequence of at least 10 nucleotides that is complementary to a target sequence. For example, the variable region or guide sequence comprises from about 10 to about 100 nucleotides and a sequence of 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to a target sequence.

In some embodiments, the variable region or guide sequence is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long and comprises a sequence of 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to a target sequence.

The variable region or guide sequence can be located on the 5′-end or the 3′-end of the guide RNA. In some embodiments, the variable region or guide sequence is at 5′-end of the guide RNA. In other words, 3′-end of the variable region is linked to the 5′-end of the gRNA, e.g., 3′-end of the variable region is linked to the 5′-N region of the guide RNA. The variable region and the 5′-N region can be linked directly (e.g., by a bond or modified or unmodified internucleoside linkage) or by a nucleotide sequence comprising from 1 to 25 nucleotides. In some preferred embodiments, the 3′-end of the variable region is linked directly to the 5′-end of the 5′-N region by a modified (e.g., phosphorothioate, imidp or MMI linkage) or unmodified (e.g., phosphodiester) internucleoside linkage. When the 5′-N region is absent, the variable region can be linked to the S1′ region directly, e.g., by a bond or modified (e.g., phosphorothioate, imidp or MMI) or unmodified (e.g., phosphodiester) internucleoside linkage.

In some embodiments, the variable region comprises a nucleotide sequence having at least 60%, 65%, 70%, 75%, 80%, 85%, 90%, or 95% identity to a nucleotide selected from the group consisting of SEQ ID NOs.: 123-314. For example, the variable region comprises a nucleotide sequence having 100% identity to any one of SEQ ID NOs: 123-314.

In some embodiments, the guide RNA comprises a direct repeat (DR) sequence linked to the guide sequence. The direct repeat sequence comprises at least one hairpin or stem loop structure. Generally, the direct repeat sequence has a minimum length of 16 nucleotides and one hairpin or stem loop. In some embodiments, the direct repeat sequence has a length longer than 16 nucleotides, preferably more than 17 nucleotides, and has two or more hairpin or stem loops. In some embodiments, the hairpin or the stem loop structure comprises at least 5, preferably 7-20 nucleotides. The direct repeat sequence can be 3′ of the spacer (guide sequence). Alternatively, the direct repeat sequence can be 5′ of the spacer (guide sequence). In some embodiments, the spacer is flanked by a direct repeat sequence at its 5′ and 3′ ends. It is noted that a guide RNA comprising a spacer flanked by a direct repeat sequence at its 5′ and 3′ ends (DR-spacer-DR) structure is typical of precursor crRNA (pre-crRNA). In some embodiments, the guide RNA comprises a truncated direct repeat sequence and a spacer sequence, which is typical of processed or mature crRNA.

The direct repeat can be at least 10 nucleotides in length. For example, the direct repeat can be at least 11 nucleotides, or at least 12 nucleotides, or at least 13 nucleotides, or at least 14 nucleotides, or at least 15 nucleotides, or at least 16 nucleotides, or at least 17 nucleotides, or at least 18 nucleotides, or at least 19 nucleotides, or at least 30 nucleotides in length. In some embodiments, the direct repeat comprises the sequence NACACC, proximal to the spacer, where N is G or T.

The guide RNA can be single-stranded or double-stranded. In some embodiments, the guide RNA is single polynucleotide chain, and optionally comprises double-stranded regions. Guide RNA that comprise a single polynucleotide chain can be referred to as a “single guide RNA.” It is noted that when the guide RNA is a single polynucleotide chain, the spacer and the tracr sequence can be located in any preferred position to each other. For example, the spacer can be located 5′ of the tracr sequence. Alternatively, the spacer can be located 3′ of the tracr sequence. In some preferred embodiments, the spacer is 5′ of the tracr sequence.

In some embodiments, the spacer and the tracr sequence of the guide RNA are present in separate polynucleotide chains. Optionally, the separate polynucleotides chains are partially hybridized to each other. Guide RNA that comprise two polynucleotide chains can be referred to as a “double guide RNA.” Generally, the 3′-end of the polynucleotide chain comprising the spacer hybridized with the 5′-end of the polynucleotide chain comprising the tracr sequence. The polynucleotide chain comprising the spacer is also referred to as “crRNA” herein, and the polynucleotide chain comprising the tracr sequence is also referred to as “tracrRNA” herein.

In some embodiments, the spacer or guide sequence is linked to a direct repeat and the tracr sequence is linked to an anti-repeat sequence. The direct repeat and anti-repeat sequences are substantially complementary to each other and can hybridize with each other to form a double-stranded region. The double-stranded region can be at least 8, or at least 9, or at least 10, or at least 11, or at least 12 base-pairs in length.

The guide RNA can also include one or more protein binding domains, e.g., for binding with or recruiting one or more gene effectors, gene activators, or gene repressors. Generally, the protein binding domain of the guide RNA comprises a scaffold that is capable of binding with a protein. In some embodiments, the protein binding domain can be an aptamer. Aptamers are oligonucleotide or peptide molecules that can bind to a specific target molecule. The aptamers can be specific to gene effectors, gene activators, or gene repressors. In some embodiments, the aptamers can be specific to a protein, which in turn is specific to and recruits/binds to specific gene effectors, gene activators, or gene repressors. The effectors, activators, or repressors can be present in the form of fusion proteins. In some embodiments, the guide RNA comprises two or more aptamer sequences that are specific to the same adaptor proteins. In some embodiments, the two or more aptamer sequences are specific to different adaptor proteins. The adaptor proteins can include, but are not limited to, MS2, PP7, Qβ, F2, GA, fr, JP501, M12, R17, BZ13, JP34, JP500, KU1, M11, MX1, TW18, VK, SP, FI, ID2, NL95, TW19, AP205, φCb5, φCb8r, φCb12r, φCb23r, 7s, and PRR1. Accordingly, in some embodiments, the aptamer is selected from binding proteins specifically binding any one of the adaptor proteins as described herein. In some embodiments, the aptamer sequence is a MS2 binding loop, Qβ binding loop, or PP7 binding loop. A detailed description of aptamers can be found, e.g., in Nowak et al., “Guide RNA engineering for versatile Cas9 functionality,” Nucl. Acid. Res., 2016 Nov. 16; 44(20):9555-9564; and WO2016205764, which are incorporated herein by reference in their entirety.

In an embodiment, the guide RNA further comprises an extension to add an RNA template. In some embodiments, a “protector nucleic acid” can be hybridized to a sequence of the guide RNA, wherein the “protector nucleic acid” is nucleic acid strand complementary to the 3′ end of the guide RNA to thereby generate a partially double-stranded guide RNA. In some embodiments, protecting mismatched bases (i.e., the bases of the guide RNA which do not form part of the guide sequence) with a perfectly complementary protector sequence decreases the likelihood of a target sequence binding to the mismatched basepairs at the 3′ end of the guide RNA. In some embodiments, additional sequences comprising an extended length can also be present within the guide RNA such that the guide RNA comprises a protector sequence within the guide RNA molecule. This “protector sequence” ensures that the guide RNA molecule comprises a “protected sequence” in addition to an “exposed sequence” (comprising the part of the guide RNA sequence hybridizing to the target sequence). In some embodiments, the guide RNA is modified by the presence of the protector nucleic acid to comprise a secondary structure such as a hairpin. Advantageously there are three or four to thirty or more, e.g., about 10 or more, contiguous base pairs having complementarity to the protected sequence, the guide RNA sequence or both. It is advantageous that the protected portion does not impede thermodynamics of the protein effector polypeptide and related system interacting with its target. By providing such an extension including a partially double stranded guide RNA, the guide RNA is considered protected and results in improved specific binding of the protein effector polypeptide/guide RNA, while maintaining specific activity.

In some embodiments, the guide RNA structure comprises a sequence predicted to comprise a hairpin consisting of a stem and a loop, wherein the stem comprises at least 10, at least 12 or at least 14 base-paired ribonucleotides, and an asymmetric bulge within 4 base pairs of the loop.

In some embodiments, the guide RNA comprises a sequence predicted to comprise a hairpin with an uninterrupted base-paired region comprising at least 8 nucleotides of a guide sequence and at least 8 nucleotides of a tracr sequence, and wherein the tracr sequence comprises, from 5′ to 3′, a first hairpin and a second hairpin, wherein the first hairpin has a longer stem than the second hairpin.

The guide RNAs can be chemically synthesized or can be generated as components of inducible systems. The inducible nature of the systems allows for spatiotemporal control of gene editing or gene expression. In some embodiments, the stimuli for the inducible systems include, e.g., electromagnetic radiation, sound energy, chemical energy, and/or thermal energy. For example, the transcription of guide RNAs can be modulated by inducible promoters, e.g., tetracycline or doxycycline controlled transcriptional activation (Tet-On and Tet-Off expression systems), hormone inducible gene expression systems (e.g., ecdysone inducible gene expression systems), and arabinose-inducible gene expression systems. Other examples of inducible systems include, e.g., small molecule two-hybrid transcription activations systems (FKBP, ABA, etc.), light inducible systems (Phytochrome, LOV domains, or cryptochrome), or Light Inducible Transcriptional Effector (LITE). These inducible systems are described, e.g., in WO 2016205764 and U.S. Pat. No. 8,795,965, both of which are incorporated herein by reference in their entirety.

The architecture of multiple RNA guides is known in the art. See, for example, International Publication Nos. WO 2014/093622 and WO 2015/070083, the entire contents of each of which are incorporated herein by reference). Thus, in some embodiments, the engineered, non-natural DNA and RNA targeting complexes described herein include multiple guide RNAs (e.g., two, three, four, five, six, seven, eight, or more guide RNAs). Sequences for RNA guides from multiple CRISPR systems are known in the art and can be searched using public databases (see, e.g., Grissa et al. (2007) Nucleic Acids Res. 35 (web server issue): W52-7; Grissa et al. (2007) BMC Bioinformatics 8: 172; Grissa et al. (2008) Nucleic Acids Res. 36 (web server issue): W145-8; and Moller and Liang (2017) PeerJ 5: e3788; see also the CRISPR database available at: crispr.i2bc.paris-saclay.fr/crispr/BLAST/CRISPRsBlast.php; and MetaCRAST available at:github.com/molleraj/MetaCRAST).

In some embodiments, an engineered, non-natural DNA and RNA targeting complexes described herein comprises two different guide RNAs targeting a first region and a second region in a target nucleic acid. In some embodiments, the first region is 5′ to the second region. In some other embodiments, the first region is 3′ to the second region.

In some embodiments, two different guide RNAs can be encoded by a single polynucleotide. For example, the polynucleotide sequence encoding a guide RNA comprises a CRarray comprising two or more guide sequences.

The guide RNA sequences can be modified in a manner that allows for formation of a complex comprising the Cas9-IID described herein and the guide RNA, and successful binding to the target, while at the same time not allowing for successful nuclease activity. These modified guide sequences are referred to as “dead guides” or “dead guide sequences.” These dead guides or dead guide sequences can be catalytically inactive or conformationally inactive with regards to nuclease activity. Dead guide sequences are typically shorter than respective guide sequences that result in active target (e.g., RNA or DNA) cleavage. In some embodiments, dead guides are 5%, 10%, 20%, 30%, 40%, or 50%, shorter than respective guide RNAs that have nuclease activity. Dead guide sequences of guide RNAs can be from 13 to 15 nucleotides in length (e.g., 13, 14, or 15 nucleotides in length), from 15 to 19 nucleotides in length, or from 17 to 18 nucleotides in length (e.g., 17 nucleotides in length). Thus, in one aspect, the disclosure provides engineered, non-natural DAN and RNA targeting system or composition comprising a functional protein effector polypeptide as described herein, and a guide RNA, wherein the guide RNA includes a dead guide sequence whereby the guide RNA is capable of hybridizing to a target sequence such that the complex is directed to a genomic locus of interest, e.g., in a cell without detectable cleavage activity. A detailed description of dead guides is described, e.g., in WO 2016094872, which is incorporated herein by reference in its entirety.

In some embodiments, a DNA and RNA targeting complex described herein exhibits a lower or decreased off-target activity, i.e., modification of a non-target sequence, as compared to the off-target activity of a complex comprising a wild-type Cas9-IID and wild-type gRNA. For example, the off-target activity of a DNA and RNA targeting complex described herein, is at least at least 2-fold, at least 3-fold, at least 4-fold, at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold lower than the off-target activity of a complex comprising a wild-type Cas9-IID and wild-type gRNA.

### Conservative Variants

In the various embodiments described herein, it is further contemplated that variants (naturally occurring or otherwise), alleles, homologs, conservatively modified variants, and/or conservative substitution variants of any of the polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

The term “amino acid substitution” refers to the replacement of at least one existing amino acid residue in a predetermined or native amino acid sequence with a different “replacement” amino acid. A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested confirm that a desired activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Conservative substitution tables providing functionally similar amino acids are also available from a variety of references (see, for e.g., Creighton, Proteins: Structures and Molecular Properties (W H Freeman & Co.; 2nd edition (December 1993)). The following eight groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) 8) Cysteine (C), Methionine (M). Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

### Split Cas9-IID Proteins

The present disclosure also provides a split version of the Cas9-IID proteins described herein. The split version of the Cas9-IID proteins can be advantageous for delivery. In some embodiments, the Cas9-IID proteins are split into two parts, which together substantially comprises a functional activity of the full length of the Cas9-IID protein. The split can be done in a way that the catalytic domain(s) are unaffected. The Cas9-IID proteins can function as a nuclease (e.g., endonuclease) or can be inactivated enzymes, which are essentially DNA or RNA-binding proteins with very little or no catalytic activity (e.g., due to mutation(s) in its catalytic domains).

In some embodiments, the nuclease lobe and a-helical lobe are expressed as separate polypeptides. Although the lobes do not interact on their own, the guide nucleic acid recruits them into a surveillance complex that recapitulates the activity of full-length Cas9-IID proteins and catalyzes site-specific DNA or RNA cleavage. The use of a modified guide nucleic acid can abrogate split-polypeptide activity by preventing dimerization, allowing for the development of an inducible dimerization system. The split CRISPR enzymes are described, e.g., in Wright, Addison V., et al. “Rational design of a split-Cas9 enzyme complex,” Proc. Natl. Acad. Sci., 112.10 (2015): 2984-2989, which is incorporated herein by reference in its entirety.

In some embodiments, the split Cas9-IID protein can be fused to a dimerization partner, e.g., by employing rapamycin sensitive dimerization domains. This allows the generation of a chemically inducible Cas9-IID proteins for temporal control of activity. The Cas9-IID proteins can thus be rendered chemically inducible by being split into two fragments and rapamycin-sensitive dimerization domains can be used for controlled reassembly of the Cas9-IID protein.

The split Cas9-IID proteins can be induced to combine to form a functional domain, e.g., nuclease domain from split Cas9-IID proteins can be inducible, e.g., light inducible or chemical inducible. Without wishing to be bound by a theory, this mechanism allows for activation of the functional domain in the Cas9-IID proteins with a known trigger. Light inducibility can be achieved by various methods known in the art, e.g., by designing a fusion complex wherein CRY2 PHR/CIBN pairing is used in split Cas9-IID proteins. See, for example, Konermann et al. “Optical control of mammalian endogenous transcription and epigenetic states,” Nature, 500.7463 (2013): 472). Chemical inducibility can be achieved, e.g., by designing a fusion complex wherein FKBP/FRB (FK506 binding protein/FKBP rapamycin binding domain) pairing is used in split Cas9-IID proteins. Rapamycin is required for forming the fusion complex, thereby activating the Cas9-IID proteins (see, e.g., Zetsche, Volz, and Zhang, “A split-Cas9 architecture for inducible genome editing and transcription modulation,” Nature Biotech., 33.2 (2015): 139-142).

The split point is typically designed in silico and cloned into the constructs. During this process, mutations can be introduced to the split enzyme and non-functional domains can be removed. In some embodiments, the two parts or fragments of the split Cas9-IID protein (i.e., the N-terminal and C-terminal fragments), can form a full Cas9-IID protein, comprising, e.g., at least 70%, at least 80%, at least 90%, at least 95%, or at least 99% of the sequence of the wild-type or full-length Cas9-IID protein.

### Self-Inactivating Cas9-IID Proteins

The Cas9-IID proteins described herein can be designed to be self-activating or self-inactivating. In some embodiments, the Cas9-IID proteins are self-inactivating. For example, a target sequence can be introduced into the Cas9-IID protein coding constructs. Thus, the Cas9-IID proteins can cleave the target sequence, as well as the construct encoding the enzyme thereby self-inactivating their expression. See, for example, Epstein, Benjamin E., and David V. Schaffer. “Engineering a Self-Inactivating CRISPR System for AAV Vectors,” Mol. Then, 24 (2016): S50, which is incorporated herein by reference in its entirety.

In some other embodiments, an additional guide nucleic acid, e.g., expressed under the control of a weak promoter (e.g., 7SK promoter), can target the nucleic acid sequence encoding the Cas9-IID protein to prevent and/or block its expression (e.g., by preventing the transcription and/or translation of the nucleic acid). The transfection of cells with vectors expressing the Cas9-IID protein and guide nucleic acid(s) that target the nucleic acid encoding the Cas9-IID protein can lead to efficient disruption of the nucleic acid encoding the Cas9-IID protein and decrease the levels of Cas9-IID protein, thereby limiting the nucleic acid modifying activity.

In some embodiments, the genome editing activity of the Cas9-IID proteins can be modulated through endogenous RNA signatures (e.g., miRNA) in mammalian cells. The Cas9-IID protein switch can be made by using a miRNA-complementary sequence in the 5′-UTR of mRNA encoding the Cas9-IID protein. The switches selectively and efficiently respond to miRNA in the target cells. Thus, the switches can differentially control the genome editing by sensing endogenous miRNA activities within a heterogeneous cell population. Therefore, the switch systems can provide a framework for cell-type selective genome editing and cell engineering based on intracellular miRNA information (Hirosawa, Moe et al. “Cell-type-specific genome editing with a microRNA-responsive CRISPR-Cas9 switch,” Nucl. Acids Res., 2017 Jul. 27; 45(13): e118).

### Nucleic Acids and Vectors Encoding Polypeptides and Guides

The disclosure also provides a polynucleotide encoding a Cas9-IID protein and/or a guide nucleic acid described herein. The skilled person will understand that, due to the degeneracy of the genetic code, a given polypeptide can be encoded by different polynucleotides. These “variants” are encompassed herein.

In some embodiments, a polynucleotide encoding a Cas9-IID protein and/or a guide nucleic acid described herein is comprised in a vector. In some embodiments, a nucleic acid sequence encoding a Cas9-IID protein and/or a guide nucleic acid described herein, or any part thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. The term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell).

A vector can include, but is not limited to, a cloning vector, an expression vector, a plasmid, phage, transposon, cosmid, chromosome, virus, virion, etc. Some exemplary vectors include pGEX, pMAL, pRIT5, E. coli expression vectors (e.g., pTrc, pET 1id, yeast expression vectors (e.g., pYepSec1, pMFa, pJRY88, pYES2, and picZ, Baculovirus vectors (e.g., for expression in insect cells such as SF9 cells) (e.g., pAc series and the pVL series), mammalian expression vectors (e.g., pCDM8 and pMT2PC).

A vector can comprise one or more (e.g., 1, or at least 2, at least 3, at least 4, at least 5, at least 6, at least 7, at least 8, at least 9, at least 10 or more) Cas9-IID protein encoding sequence(s), and/or one or more (e.g., 1, or at least 2, at least 3, at least 4, at least 5, at least 6, at least 7, at least 8, at least 9, at least 10, at least 12, at least 14, at least 16, at least 32, at least 48, at least 50 or more) guide nucleic acid encoding sequences. In a single vector there can be a promoter for each RNA coding sequence. Alternatively, or additionally, in a single vector, there can be a promoter controlling (e.g., driving transcription and/or expression) multiple RNA encoding sequences.

In some embodiments, the vector comprises: a first regulatory element operably linked to a nucleotide sequence encoding a Cas9-IID protein described herein, and a second regulatory element operably linked to a nucleotide sequence encoding a guide nucleic acid described herein.

Examples of regulatory elements include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. [0561] Examples of promoters include one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the P-actin promoter, the phosphoglycerol kinase (PGK) promoter, and the EF1a promoter.

In some embodiments, the promoter is an inducible promoter. In some embodiments, the promoter is a cell-specific promoter, such as Syn and CamKIIa for neuronal cell types, or thyroxine binding globulin (TBG) for hepatocyte expression. In some embodiments, the promoter is an organism-specific promoter. Suitable promoters are known in the art and include, for example, a pol I promoter, a pol II promoter, a pol III promoter. In some embodiments, short RNAs such as the guide nucleic acid are effectively expressed using a pol III promoter, which includes a U6 promoter, a H1 promoter, a 7SK promoter. In some embodiments, the promoter is prokaryotic, such as a T7 promoter. In some embodiments, the promoters are eukaryotic and include retroviral Rous sarcoma virus LTR promoter, a cytomegalovirus (CMV) promoter, a SV40 promoter, a dihydrofolate reductase promoter, a b-actin promoter, elongation factor 1 alpha promoter, elongation factor 1 alpha short promoter, SV40 promoter, and the synthetic CAG promoter. In some embodiments, the termination signals for induction of mRNA polyadenylation include, but are not limited to, SV40, hGH, and bGH.

In some embodiments, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

In some embodiments, the sequence encoding the Cas9-IID protein and/or a guide nucleic acid described herein is codon-optimized, e.g., the native or wild-type sequence of the nucleic acid sequence has been altered or engineered to include alternative codons such that altered or engineered nucleic acid encodes the same polypeptide or expression product as the native/wild-type sequence, but will be transcribed and/or translated at an improved efficiency in a desired expression system. In some embodiments, the expression system is an organism other than the source of the native/wild-type sequence (or a cell obtained from such organism). In some embodiments, the vector and/or nucleic acid sequence described herein is codon-optimized for expression in a mammal or mammalian cell, e.g., a mouse, a murine cell, or a human cell. In some embodiments, the vector and/or nucleic acid sequence described herein is codon-optimized for expression in a human cell. In some embodiments, the vector and/or nucleic acid sequence described herein is codon-optimized for expression in a yeast or yeast cell. In some embodiments, the vector and/or nucleic acid sequence described herein is codon-optimized for expression in a bacterial cell. In some embodiments, the vector and/or nucleic acid sequence described herein is codon-optimized for expression in an E. coli cell.

As used herein, the term “expression vector” refers to a vector that directs expression of an RNA or polypeptide from sequences operably linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

In some embodiments, the vector can be a viral vector. As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art. Some exemplary viral vectors amenable to the invention include, but are not limited to, adeno associated virus (AAV such as AAV-1, AAV-2, AAV-3, AAV-4, AAV-5, AAV-6, AAV-8, and AAV-9), adenoviruses lentiviruses (such as human immunodeficiency virus and equine infectious anemia virus), plat viral vectors (such as geminivirus (e.g., cabbage leaf curl virus, bean yellow dwarf virus, wheat dwarf virus, tomato leaf curl virus, maize streak virus, tobacco leaf curl virus, and tomato golden mosaic virus), nanovirus (e.g., Faba bean necrotic yellow virus), tobravirus (e.g., tobacco rattle virus, tobacco mosaic virus), potexvirus (e.g., potato virus X), and hordeivirus (e.g., barley stripe mosaic virus).

In some embodiments, the vector comprises: a first regulatory element operably linked to a nucleotide sequence encoding a Cas9-IID protein described herein, and a second regulatory element operably linked to a nucleotide sequence encoding a guide nucleic acid described herein.

Examples of regulatory elements include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. [0561] Examples of promoters include one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the P-actin promoter, the phosphoglycerol kinase (PGK) promoter, and the EF1a promoter.

It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

It is noted that any reference to a Cas9-IID protein or a guide nucleic acid includes polynucleotides or vectors encoding same. Particularly, any reference to a Cas9-IID protein or a guide nucleic acid includes polynucleotides or vectors encoding same, where the polynucleotide or vector is operably linked to one or more regulatory elements, such as a promoter.

### Modified Cells

The disclosure also provides cell comprising one or more components of the engineered, non-natural DNA and RNA targeting complexes described herein or a polynucleotide (e.g., a vector) encoding the same. Also provided herein are cells modified by the engineered, non-natural DNA and RNA targeting complexes described herein, and cell cultures, tissues, organs, organism comprising such cells or progeny thereof.

A modified cell or a cell comprising one or more components of the engineered, non-natural DNA and RNA targeting complexes described herein or a polynucleotide (e.g., a vector) encoding the same can be a prokaryotic cell or a eukaryotic cell. The cell can be a mammalian cell. The mammalian cell can be a non-human primate, bovine, porcine, rodent or mouse cell. The cell can be a non-mammalian eukaryotic cell such as poultry, fish or shrimp. The cell can be a therapeutic T cell or antibody-producing B-cell. The cell can also be a plant cell. The plant cell can be of a crop plant such as cassava, corn, sorghum, wheat, or rice. The plant cell can also be of an algae, tree or vegetable. The modification introduced to the cell using the DNA and RNA targeting systems, compositions and methods described herein can be such that the cell and progeny of the cell are altered for improved production of biologic products such as an antibody, starch, alcohol or other desired cellular output. The modification introduced to the cell using the DNA and RNA targeting systems, compositions and methods described herein can be such that the cell and progeny of the cell include an alteration that changes the biologic product produced.

### Synthesis of Cas9-IID Proteins and Guide Nucleic Acids

Recombinant methods can be used for producing Cas9-IID proteins and/or guide nucleic acids described herein. For example, host cells can be employed in a method of producing a Cas9-IID protein and/or a guide nucleic acid described herein. Accordingly, the disclosure also provides a host cell comprising a polynucleotide or a plasmid or vector encoding a Cas9-IID protein and/or a guide nucleic acid described herein. A host cell can be a prokaryotic or eukaryotic host cell. Exemplary host cells include, but are not limited to, bacterial cells, yeast cells, plant cell, animal (including insect) or human cells.

Generally, the method comprises: culturing a host cell comprising a polynucleotide described herein or a plasmid or vector described herein under conditions such that the Cas9-IID protein and/or the guide nucleic acid is expressed; and optionally recovering the Cas9-IID protein and/or the guide nucleic acid from the culture medium. The Cas9-IID protein and/or the guide nucleic acid can be concentrated and purified by a variety of biochemical and chromatographic methods, including methods utilizing differences in size, charge, hydrophobicity, solubility, specific affinity, etc. between the desired product (e.g., the Cas9-IID protein and/or the guide nucleic acid) and other substances in the cell culture medium. In some embodiments, the Cas9-IID protein and/or the guide nucleic acid is secreted from the host cells.

The Cas9-IID protein described herein can be produced as recombinant molecules in prokaryotic or eukaryotic host cells, such as bacteria, yeast, plant, animal (including insect) or human cell lines or in transgenic animals. Recombinant methods of producing a polypeptide through the introduction of a vector including nucleic acid encoding the polypeptide into a suitable host cell is well known in the art, such as is described in Sambrook et al., Molecular Cloning: A Laboratory Manual, 2d Ed, Vols 1 to 8, Cold Spring Harbor, NY (1989); M. W. Pennington and B. M. Dunn, Methods in Molecular Biology: Peptide Synthesis Protocols, Vol 35, Humana Press, Totawa, NJ (1994), contents of both of which are herein incorporated by reference.

The production of Cas9-IID proteins at high levels in suitable host cells requires the assembly of the polynucleotides encoding such Cas9-IID proteins into efficient transcriptional units together with suitable regulatory elements in a recombinant expression vector that can be propagated in various expression systems according to methods known to those skilled in the art. Efficient transcriptional regulatory elements could be derived from viruses having animal cells as their natural hosts or from the chromosomal DNA of animal cells. For example, promoter-enhancer combinations derived from the Simian Virus 40, adenovirus, BK polyoma virus, human cytomegalovirus, or the long terminal repeat of Rous sarcoma virus, or promoter-enhancer combinations including strongly constitutively transcribed genes in animal cells like beta-actin or GRP78 can be used. In order to achieve stable high levels of mRNA, the transcriptional unit should contain in its 3′-proximal part a DNA region encoding a transcriptional termination-polyadenylation sequence. Generally, this sequence can be derived from the Simian Virus 40 early transcriptional region, the rabbit beta-globin gene, or the human tissue plasminogen activator gene.

The vector is transfected into a suitable host cell line for expression of the Cas9-IID protein and/or the guide nucleic acid. Examples of cell lines that can be used to prepare the Cas9-IID protein and/or the guide nucleic acid described herein include, but are not limited to monkey COS-cells, mouse L-cells, mouse C127-cells, hamster BHK-21 cells, human embryonic kidney 293 cells, and hamster CHO-cells.

The expression vector encoding the Cas9-IID protein and/or the guide nucleic acid can be introduced in several different ways. For instance, the expression vectors can be created from vectors based on different animal viruses. Examples of these are vectors based on baculovirus, vaccinia virus, adenovirus, and preferably bovine papilloma virus.

The transcription units encoding the corresponding DNAs can also be introduced into animal cells together with another recombinant gene, which may function as a dominant selectable marker in these cells in order to facilitate the isolation of specific cell clones, which have integrated the recombinant DNA into their genome. Examples of this type of dominant selectable marker genes are Tn5 amino glycoside phosphotransferase, conferring resistance to geneticin (G418), hygromycin phosphotransferase, conferring resistance to hygromycin, and puromycin acetyl transferase, conferring resistance to puromycin. The recombinant expression vector encoding such a selectable marker can reside either on the same vector as the one encoding the cDNA of the desired protein, or it can be encoded on a separate vector which is simultaneously introduced and integrated to the genome of the host cell, frequently resulting in a tight physical linkage between the different transcription unit.

Other types of selectable marker genes, which can be used together with the cDNA of the desired protein are based on various transcription units encoding dihydrofolate reductase (dhfr). After introduction of this type of gene into cells lacking endogenous dhfr-activity, preferentially CHO-cells (DUKX-B11, DG-44) it will enable these to grow in media lacking nucleosides. An example of such a medium is Ham's F12 without hypoxanthine, thymidin, and glycine. These dhfr-genes can be introduced together with the Kazal-type serine protease inhibitors' cDNA transcriptional units into CHO-cells of the above type, either linked on the same vector or on different vectors, thus creating dhfr-positive cell lines producing recombinant protein.

If the above cell lines are grown in the presence of the cytotoxic dhfr-inhibitor methotrexate, new cell lines resistant to methotrexate will emerge. These cell lines may produce recombinant protein at an increased rate due to the amplified number of linked dhfr and the desired protein's transcriptional units. When propagating these cell lines in increasing concentrations of methotrexate (1-10000 nM), new cell lines can be obtained which produce the desired protein at a very high rate.

The above cell lines producing the desired Cas9-IID protein and/or guide nucleic acid can be grown on a large scale, either in suspension culture or on various solid supports. Examples of these supports are micro carriers based on dextran or collagen matrices, or solid supports in the form of hollow fibers or various ceramic materials. When grown in cell suspension culture or on micro carriers the culture of the above cell lines can be performed either as a batch culture or as a perfusion culture with continuous production of conditioned medium over extended periods of time. Thus, according to the present invention, the above cell lines are well suited for the development of an industrial process for the production of the desired Cas9-IID protein and/or the guide nucleic acid.

An example of such purification is the adsorption of the Cas9-IID protein to a monoclonal antibody or a binding peptide, which is immobilized on a solid support. After desorption, the protein can be further purified by a variety of chromatographic techniques based on the above properties.

Exemplary genera of yeast contemplated to be useful in the production of the Cas9-IID protein and/or described herein as hosts are Pichia (formerly classified as Hansenula), Saccharomyces, Kluyveromyces, Aspergillus, Candida, Torulopsis, Torulaspora, Schizosaccharomyces, Citeromyces, Pachysolen, Zygosaccharomyces, Debaromyces, Trichoderma, Cephalosporium, Humicola, Mucor, Neurospora, Yarrowia, Metschunikowia, Rhodosporidium, Leucosporidium, Botryoascus, Sporidiobolus, Endomycopsis, and the like. Genera include those selected from the group consisting of Saccharomyces, Schizosaccharomyces, Kluyveromyces, Pichia and Torulaspora. Examples of Saccharomyces spp. are S. cerevisiae, S. italicus and S. rouxii.

Suitable promoters for S. cerevisiae include those associated with the PGKI gene, GAL1 or GAL10 genes, CYCI, PHO5, TRPI, ADHI, ADH2, the genes for glyceral-dehyde-3-phosphate dehydrogenase, hexokinase, pyruvate decarboxylase, phos-phofructokinase, triose phosphate isomerase, phosphoglucose isomerase, glucokinase, alpha-mating factor pheromone, the PRBI, the GUT2, the GPDI promoter, and hybrid promoters involving hybrids of parts of 5′ regulatory regions with parts of 5′ regulatory regions of other promoters or with upstream activation sites (e.g. the promoter of EP-A-258 067).

Convenient regulatable promoters for use in Schizosaccharomyces pombe are the thiamine-repressible promoter from the nmt gene as described by Maundrell (Maundrell K. 1990. Nmtl of fission yeast. A highly transcribed gene completely repressed by thiamine. J. Biol. Chem. 265:10857-10864) and the glucose repressible jbpl gene promoter as described by Hoffman and Winston (Hoffman C S and Winston F. 1990. Isolation and characterization of mutants constitutive for expression of the fbpl gene of Schizosaccharomyces pombe. Genetics 124:807-816).

The transcription termination signal may be the 3′ flanking sequence of a eukaryotic gene which contains proper signals for transcription termination and polyadenylation. Suitable 3′ flanking sequences may, for example, be those of the gene naturally linked to the expression control sequence used, i.e. may correspond to the promoter. Alternatively, they may be different in which case the termination signal of the S. cerevisiae ADHI gene is optionally used.

Exemplary expression systems for the production of the Cas9-IID protein and/or the guide nucleic acids described herein in bacteria include Bacillus subtilis, Bacillus brevis, Bacillus megaterium, Caulobacter crescentus, and, most importantly, Escherichia coli BL21 and E. coli K12 and their derivatives. Convenient promoters include but are not limited to trc promoter, tac promoter, lac promoter, lambda phage promoter P_{L}, the L-arabinose inducible araBAD promoter, the L-rhamnose inducible rhaP promoter, and the anhydrotetracycline-inducible tetA promoter/operator.

In some embodiments, the Cas9-IID protein or the polynucleotide encoding the Cas9-IID protein, further comprises a signal sequence and/or a leader sequence. A signal sequence (sometimes referred to as signal peptide, targeting signal, localization signal, localization sequence, or transit peptide) is a short “pre-peptide” (usually 16-30 amino acids long) present at the N-terminus (or occasionally non-classically at the C-terminus or internally) of most newly synthesized secretory proteins. The signal sequence facilitates translocation of the expressed polypeptide to which it is attached into the endoplasmic reticulum. Signal peptide typically comprises a positively charged n-region, a hydrophobic h-region, and a neutral, polar c-region. At the end of the signal sequence, there is typically a stretch of amino acids that is recognized and cleaved by a signal peptidase and therefore named the cleavage site. The signal sequence is normally cleaved off in the course of the secretion process.

In some embodiments, a polynucleotide encoding the Cas9-IID protein described herein can be fused to signal sequences which will direct the localization of the Cas9-IID protein to particular compartments of a prokaryotic cell and/or direct the secretion of a protein of the invention from a prokaryotic cell. For example, in E. coli, one may wish to direct the expression of the protein to the periplasmic space. Examples of signal sequences or proteins (or fragments thereof) to which the proteins of the invention may be fused in order to direct the expression of the polypeptide to the periplasmic space of bacteria include, but are not limited to, the pelB signal sequence, the maltose binding protein signal sequence, the ompA signal sequence, the signal sequence of the periplasmic E. coli heat-labile enterotoxin B-subunit, and the signal sequence of alkaline phosphatase. Several vectors are commercially available for the construction of fusion proteins which will direct the localization of a protein, such as the pMAL series of vectors (NEW ENGLAND BIOLABS).

The expression of the Cas9-IID proteins from a polypeptide or vector encoding same can be modulated by inducible promoters, e.g., tetracycline or doxycycline controlled transcriptional activation (Tet-On and Tet-Off expression system), hormone inducible gene expression system (e.g., an ecdysone inducible gene expression system), and an arabinose-inducible gene expression system. When delivered as RNA, expression of the Cas9-IID proteins can be modulated via a riboswitch, which can sense a small molecule like tetracycline. See, for example, Goldfless, Stephen J. et al. “Direct and specific chemical control of eukaryotic translation with a synthetic RNA-protein interaction,” Nucl. Acids Res., 40.9 (2012): e64-e64). Some exemplary methods and constructs for inducible CRISPR enzymes and inducible CRISPR systems amenable to the present invention are described, for example, in U.S. Pat. No. 8,871,445, US20160208243, and WO2016205764, each of which is incorporated herein by reference in its entirety.

### Nucleic Acid Modifications

One or more chemical or nucleic acid modifications can be applied to polynucleotides, described herein, guide nucleic acids described herein, polynucleotides encoding a Cas9-IID protein described herein, or polynucleotides encoding a guide nucleic acid described herein. Exemplary nucleic acid modifications include, but are not limited to, nucleobase modifications, sugar modifications, inter-sugar linkage modifications, conjugates (e.g., ligands), and any combinations thereof. Nucleic acid modifications also include unnatural, or degenerate nucleobases.

Exemplary modified nucleobases include, but are not limited to, inosine, xanthine, hypoxanthine, nubularine, isoguanosine, tubercidin, and substituted or modified analogs of adenine, guanine, cytosine and uracil, such as 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and 0-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine,7-deazaadenine, N6, N6-dimethyladenine, 2,6-diaminopurine, 5-amino-allyl-uracil, N3-methyluracil, substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3carboxypropyl)uracil, 3-methylcytosine, 5-methylcytosine, N⁴-acetyl cytosine, 2-thiocytosine, N6-methyladenine, N6-isopentyladenine, 2-methylthio-N6-isopentenyladenine, N-methylguanines, or O-alkylated bases. Further purines and pyrimidines include those disclosed in U.S. Pat. No. 3,687,808, those disclosed in the Concise Encyclopedia of Polymer Science and Engineering, pages 858-859, Kroschwitz, J. I., ed. John Wiley & Sons, 1990, and those disclosed by Englisch et al., Angewandte Chemie, International Edition, 1991, 30, 613.

In some embodiments, a modified nucleobase can be selected from the group consisting of: inosine, xanthine, hypoxanthine, nubularine, isoguanosine, tubercidin, 2-(halo)adenine, 2-(alkyl)adenine, 2-(propyl)adenine, 2-(amino)adenine, 2-(aminoalkyl)adenine, 2-(aminopropyl)adenine, 2-(methylthio)-N⁶-(isopentenyl)adenine, 6-(alkyl)adenine, 6-(methyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N⁶-(isopentyl)adenine, N⁶-(methyl)adenine, N⁶, N⁶-(dimethyl)adenine, 2-(alkyl)guanine,2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, 5-(trifluoromethyl)cytosine, 6-(azo)cytosine, N⁴-(acetyl)cytosine, 3-(3-amino-3-carboxypropyl)uracil, 5-ethynyl-2′-deoxyuridine, 2-(thio)uracil,5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N³-(methyl)uracil, 5-uracil (i.e., pseudouracil), 2-(thio)pseudouracil,4-(thio)pseudouracil,2,4-(dithio)psuedouracil,5-(alkyl)pseudouracil, 5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5-(alkyl)-2,4-(dithio)pseudouracil, 5-(methyl)-2,4-(dithio)pseudouracil, 1-substituted pseudouracil, 1-substituted 2-(thio)-pseudouracil, 1-substituted 4-(thio)pseudouracil, 1-substituted 2,4-(dithio)pseudouracil, 1-(aminocarbonylethylenyl)-pseudouracil, 1-(aminocarbonylethylenyl)-2-(thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylamino-carbonylethylenyl)-2-(thio)-pseudouracil, 1-(aminoalkylaminocarbonylethylenyl)-4-(thio)pseudouracil, 1-(aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidin, isoguanosine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5-nitroindole, 3-nitropyrrole, 6-(aza)pyrimidine, 2-(amino)purine, 2,6-(diamino)purine, 5-substituted pyrimidines, N²-substituted purines, N⁶-substituted purines, O⁶-substituted purines, substituted 1,2,4-triazoles, and any O-alkylated or N-alkylated derivatives thereof.

In some embodiments, a nucleic acid modification can include a non-natural or modified nucleobase.

Exemplary sugar modified nucleotides include, but are not limited to, 2′-O-methyl (2′-OMe) nucleotides, 2′-fluoro (2′-F) nucleotides, 3′-fluoro nucleotides, 3′-OMe nucleotides, bridged nucleic acid (BNA) nucleotides (e.g., 2′-0,4′-C-methylene (locked nucleic acid, LNA) nucleotides, 2′-O,4′-C-ethylene (locked nucleic acid, ENA) nucleotides, 5′-methyl-BNA, cEt BNA, cMOE BNA, oxy amino BNA and vinyl-carbo BNA), anhydrohexitol (1,5-anhydrohexitol nucleic acid, HNA) nucleotides, cyclohexene (Cyclohexene nucleic acid, CeNA) nucleotides, 2′-methoxyethyl (2′-MOE) nucleotides, 2′-O-allyl nucleotides, 2′-C-allyl ribose nucleotides, 2′-O—N-methylacetamido (2′-O-NMA) nucleotides, a 2′-O-dimethylaminoethoxyethyl (2′-O-DMAEOE) nucleotides, 2′-O-aminopropyl (2′-O-AP) nucleotides, 2′-F arabinose (2′-ara-F) nucleotides, threose (Threose nucleic acid, TNA) nucleotides, and acyclic nucleotides (e.g., peptide nucleic acid (PNA), unlocked nucleic acids (UNA), 2,3-dihydroxylpropyl (glycol nucleic acid, GNA)), and 2′-deoxy (2′-H); a modified internucleoside linkage; a non-natural or modified nucleobase; or a combination thereof.

In some embodiments, a sugar modified nucleotides can be a 2′-OMe nucleotide, 2′-F nucleotide, 2′-MOE nucleotide, BNA (e.g., LNA or ENA) nucleotide, UNA nucleotide, GNA nucleotide,

In some embodiments, a nucleic acid modification can include replacement or modification of an inter-sugar linkage, i.e., a modified internucleoside linkage. Exemplary inter-sugar linkage modifications include, but are not limited to, phosphotriesters, methylphosphonates, phosphoramidate, phosphorothioates, methylenemethylimino, thiodiester, thionocarbamate, siloxane, N,N′-dimethylhydrazine (—CH2-N(CH3)-N(CH3)-), amide-3 (3′-CH₂—C(═O)—N(H)-5′) and amide-4 (3′-CH₂—N(H)—C(═O)-5′), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methylenecarbonylamino, imidophosphoramidate (imidp, i.e.,

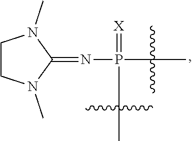

where X is O or S, preferably X is O), methylenemethylimino (MMI, 3′-CH₂—N(CH₃)—O-5′), methylenehydrazo, methylenedimethylhydrazo, methyleneoxymethylimino, ethers (C3′-O—C5′), thioethers (C3′-S—C5′), thioacetamido (C3′-N(H)—C(═O)—CH₂—S—C5′, C3′-O—P(O)—O—SS—C5′, C3′—CH₂—NH—NH—C5′, 3′-NHP(O)(OCH₃)—O-5′ and 3′-NHP(O)(OCH₃)—O-5′).

Backbone modifications such as phosphorothioates modify the charge on the phosphate backbone and can aid in the delivery and nuclease resistance of the oligonucleotide (see, e.g., Eckstein, “Phosphorothioates, essential components of therapeutic oligonucleotides,” Nucl. Acid Ther., 24 (2014), pp. 374-387). Modifications of sugars, such as 2′-O-methyl (2′-OMe), 2′-F, and locked nucleic acid (LNA), can enhance both base pairing and nuclease resistance (see, e.g., Allerson et al., “Fully 2′-modified oligonucleotide duplexes with improved in vitro potency and stability compared to unmodified small interfering RNA,” J Med. Chem., 48.4 (2005): 901-904). Chemically modified bases such as 2-thiouridine or N6-methyladenosine, among others, can allow for either stronger or weaker base pairing (see, e.g., Bramsen et al., “Development of therapeutic-grade small interfering RNAs by chemical engineering,” Front. Genet. 2012 Aug. 20; 3: 154). Additionally, the guide nucleic acid is amenable to both 5′ and 3′ end conjugations with a variety of functional moieties including, but not limited to, targeting ligands, fluorescent dyes, polyethylene glycol, or proteins.

In some embodiments of any one of the aspects described herein, each modified internucleoside linkage can be selected independently from the group consisting of phosphorothioates (R, S, or racemic), phosphorodithioates, methylenemethylimino (MMI, 3′-CH₂—N(CH₃)—O-5′), phosphotriesters, alkylphosphonates (e.g., methylphosphonates), phosphoramidate, methylenemethylimino (—CH₂—N(CH₃)—O—CH₂—), thiodiester (—O—C(O)—S—), thionocarbamate (—O—C(O)(NH)—S—), siloxane (—O—Si(H)₂—O— and dialkylsiloxane), N,N′-dimethylhydrazine (—CH₂—N(CH₃)—N(CH₃)—), amide-3 (3′-CH₂—C(═O)—N(H)-5′), amide-4 (3′-CH₂—N(H)—C(═O)-5′)), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methykenecarbonylamino, methylenehydrazo, methylenedimethylhydrazo, methyleneoxymethylimino, ethers (C3′-O—C5′), thioethers (C3′-S—C5′), thioacetamido (C3′-N(H)—C(═O)—CH₂—S—C5′, C3′-O—P(O)—O—SS—C5′), C3′-CH₂—NH—NH—C5′, 3′-NHP(O)(OCH₃)—O-5′, 3′—NHP(O)(OCH₃)—O-5′), 2′->5′ internucleoside linkages, 2′->3′ internucleoside linkages, 3′->3′ internucleoside linkages, 5′->5′ internucleoside linkages, and imidophosphoramidate (“imidp”) linkage

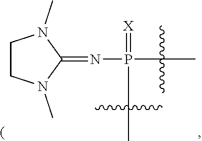

where X is O or S, preferably X is O).

A summary of chemical modifications amenable to the guide nucleic acids described herein can be found, e.g., in Kelley et al., “Versatility of chemically synthesized guide RNAs for CRISPR-Cas9 genome editing,” J. Biotechnol. 2016 Sep. 10; 233:74-83; WO 2016205764; and U.S. Pat. No. 8,795,965 B2; each which is incorporated by reference in its entirety.

In some embodiments, the Cas9-IID proteins include at least one (e.g., 1, 2, 3, 4, 5, 6, 7, 8, 9, or 10) Nuclear Localization Signal (NLS) attached to the N-terminal or C-terminal of the protein. Non-limiting examples of NLSs include an NLS sequence derived from: the NLS of the SV40 virus large T-antigen, having the amino acid sequence PKKKRKV (SEQ ID NO: 107); the NLS from nucleoplasmin (e.g., the nucleoplasmin bipartite NLS with the sequence KRPAATKKAGQAKKKK (SEQ ID NO: 108)); the c-myc NLS having the amino acid sequence PAAKRVKLD (SEQ ID NO: 109) or RQRRNELKRSP (SEQ ID NO: 110); the hRNPA1 M9 NLS having the sequence NQ S SNF GPMKGGNF GGRS S GP Y GGGGQ YF AKPRN Q GGY (SEQ ID NO: 111); the sequence RMRIZFKNKGKDTAELRRRRVEV S VELRKAKKDEQILKRRNV (SEQ ID NO: 112) of the IBB domain from importin-alpha; the sequences VSRKRPRP (SEQ ID NO: 113) and PPKKARED (SEQ ID NO: 114) of the myoma T protein; the sequence PQPKKKPL (SEQ ID NO: 115) of human p53; the sequence SALIKKKKKMAP (SEQ ID NO: 116) of mouse c-abl IV; the sequences DRLRR (SEQ ID NO: 117) and PKQKKRK(SEQ ID NO: 118) of the influenza virus NS1; the sequence RKLKKKIKKL (SEQ ID NO: 119) of the Hepatitis virus delta antigen; the sequence REKKKFLKRR (SEQ ID NO: 120) of the mouse Mxl protein; the sequence KRKGDEVDGVDEVAKKKSKK (SEQ ID NO: 121) of the human poly(ADP-ribose) polymerase; and the sequence RKCLQAGMNLEARKTKK (SEQ ID NO: 122) of the human glucocorticoid receptor. In some embodiments, the CRISPR-associated protein includes at least one (e.g., 1, 2, 3, 4, 5, 6, 7, 8, 9, or 10) Nuclear Export Signal (NES) attached the N-terminal or C-terminal of the protein. In a preferred embodiment a C-terminal and/or N-terminal NLS or NES is attached for optimal expression and nuclear targeting in eukaryotic cells, e.g., human cells.

In some embodiments, the Cas9-IID proteins described herein are mutated at one or more amino acid residues to alter one or more functional activities. For example, in some embodiments, the Cas9-IID proteins is mutated at one or more amino acid residues to alter its helicase activity. In some embodiments, the Cas9-IID proteins is mutated at one or more amino acid residues to alter its nuclease activity (e.g., endonuclease activity or exonuclease activity). In some embodiments, the Cas9-IID proteins is mutated at one or more amino acid residues to alter its ability to functionally associate with a RNA guide. In some embodiments, the Cas9-IID proteins is mutated at one or more amino acid residues to alter its ability to functionally associate with a target nucleic acid.

In some embodiments, the Cas9-IID proteins described herein are capable of cleaving a target nucleic acid molecule. In some embodiments, the Cas9-IID cleaves both strands of the target nucleic acid molecule. However, in some embodiments, the Cas9-IID proteins is mutated at one or more amino acid residues to alter its cleaving activity. For example, in some embodiments, the Cas9-IID may comprise one or more mutations that render the enzyme incapable of cleaving a target nucleic acid. In other embodiments, the Cas9-IID protein may comprise one or more mutations such that the enzyme is capable of cleaving a single strand of the target nucleic acid (i.e., nickase activity). In some embodiments, the Cas9-IID proteins are capable of cleaving the strand of the target nucleic acid that is complementary to the strand to which the RNA guide hybridizes. In some embodiments, the Cas9-IID proteins are capable of cleaving the strand of the target nucleic acid to which the RNA guide hybridizes.

In some embodiments, a Cas9-IID proteins described herein can be engineered to include a deletion in one or more amino acid residues to reduce the size of the enzyme while retaining one or more desired functional activities (e.g., nuclease activity and the ability to interact functionally with a RNA guide). The truncated Cas9-IID may be advantageously used in combination with delivery systems having load limitations.

Nucleic acids encoding the proteins (e.g., a CRISPR-associated protein) and RNA guides (e.g., a crRNA) described herein are also provided. In some embodiments, the nucleic acid is a synthetic nucleic acid. In some embodiments, the nucleic acid is a DNA molecule. In some embodiments, the nucleic acid is an RNA molecule (e.g., an mRNA molecule). In some embodiments, the mRNA is capped, polyadenylated, substituted with 5-methylcytidine, substituted with pseudouridine, or a combination thereof. In some embodiments, the nucleic acid (e.g., DNA) is operably-linked to a regulatory element (e.g., a promoter) to control the expression of the nucleic acid. In some embodiments, the promoter is a constitutive promoter.

In some embodiments, the promoter is an inducible promoter. In some embodiments, the promoter is a cell-specific promoter, such as Syn and CamKIIa for neuronal cell types, or thyroxine binding globulin (TBG) for hepatocyte expression. In some embodiments, the promoter is an organism-specific promoter. Suitable promoters are known in the art and include, for example, a pol I promoter, a pol II promoter, a pol III promoter. In some embodiments, short RNAs such as the RNA guide are effectively expressed using a pol III promoter, which includes a U6 promoter, a H1 promoter, a 7SK promoter. In some embodiments, the promoter is prokaryotic, such as a T7 promoter. In some embodiments, the promoters are eukaryotic and include retroviral Rous sarcoma virus LTR promoter, a cytomegalovirus (CMV) promoter, a SV40 promoter, a dihydrofolate reductase promoter, a b-actin promoter, elongation factor 1 alpha promoter, elongation factor 1 alpha short promoter, SV40 promoter, and the synthetic CAG promoter. In some embodiments, the termination signals for induction of mRNA polyadenylation include, but are not limited to, SV40, hGH, and bGH.

In some embodiments, the nucleic acid(s) are present in a vector (e.g., a viral vector or a phage). The vectors can include one or more regulatory elements that allow for the propagation of the vector in a cell of interest (e.g., a bacterial cell or a mammalian cell). In some embodiments, the vector includes a nucleic acid encoding a single component of a CRISPR-associated (Cas) system described herein. In some embodiments, the vector includes multiple nucleic acids, each encoding a component of a CRISPR-associated (Cas) system described herein.

In one aspect, the present disclosure provides nucleic acid sequences that are at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% identical to the nucleic sequences described herein. In another aspect, the present disclosure also provides amino acid sequences that are at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% identical to the amino acid sequences described herein.

In some embodiments, the nucleic acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are the same as the sequences described herein.

In some embodiments, the nucleic acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from the sequences described herein.

In some embodiments, the amino acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as the sequences described herein. In some embodiments, the amino acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from the sequences described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In general, the length of a reference sequence aligned for comparison purposes should be at least 80% of the length of the reference sequence, and in some embodiments is at least 90%, 95%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

In some embodiments, the Cas9-IID proteins described herein can be fused to one or more peptide tags, including a His-tag, GST-tag, FLAG-tag, or myc-tag. In some embodiments, the Cas9-IID proteins or accessory proteins described herein can be fused to a detectable moiety such as a fluorescent protein (e.g., green fluorescent protein or yellow fluorescent protein). In those embodiments where a tag is fused to a CRISPR-associated protein, such tag may facilitate affinity-based and/or charge-based purification of the CRISPR-associated protein, e.g., by liquid chromatography or bead separation utilizing an immobilized affinity or ion-exchange reagent. As a non-limiting example, a recombinant CRISPR-associated protein of this disclosure (such as a Cas12g) comprises a polyhistidine (His) tag, and for purification is loaded onto a chromatography column comprising an immobilized metal ion (e.g. a Zn²⁺, Ni²⁺, Cu²⁺ ion chelated by a chelating ligand immobilized on the resin, which resin may be an individually prepared resin or a commercially available resin) or ready to use column such as the HisTrap FF column commercialized by GE Healthcare Life Sciences, Marlborough, Massachusetts). Following the loading step, the column is optionally rinsed, e.g., using one or more suitable buffer solutions, and the His-tagged protein is then eluted using a suitable elution buffer. Alternatively, or additionally, if the recombinant CRISPR-associated protein of this disclosure utilizes a FLAG-tag, such protein may be purified using immunoprecipitation methods known in the industry. Other suitable purification methods for tagged Cas9-IID proteins or accessory proteins of this disclosure will be evident to those of skill in the art.

The Cas9-IID proteins described herein can be delivered or used as either nucleic acid molecules or polypeptides. When nucleic acid molecules are used, the nucleic acid molecule encoding the Cas9-IID proteins can be codon-optimized, as described in further detail below. The nucleic acid can be codon optimized for use in any organism of interest, in particular human cells or bacteria. For example, the nucleic acid can be codon-optimized for any non-human eukaryote including mice, rats, rabbits, dogs, livestock, or non-human primates. Codon usage tables are readily available, for example, at the “Codon Usage Database” available online with a search for kazusa.or.jp/codon/and these tables can be adapted in a number of ways. See Nakamura et al. Nucl. Acids Res. 28:292 (2000), which is incorporated herein by reference in its entirety. Computer algorithms for codon optimizing a particular sequence for expression in a particular host cell are also available, such as GENE FORGE (APTAGEN; Jacobus, PA).

In some instances, nucleic acids of this disclosure which encode Cas9-IID proteins for expression in eukaryotic (e.g., human, mammalian, etc.) cells include one or more introns, i.e., one or more non-coding sequences comprising, at a first end (e.g., a 5′ end), a splice-donor sequence and, at second end (e.g., the 3′ end) a splice acceptor sequence. Any suitable splice donor/splice acceptor can be used in the various embodiments of this disclosure, including without limitation simian virus 40 (SV40) intron, beta-globin intron, and synthetic introns. Alternatively, or additionally, nucleic acids of this disclosure encoding Cas9-IID proteins or accessory proteins may include, at a 3′ end of a DNA coding sequence, a transcription stop signal such as a polyadenylation (poly A) signal. In some instances, the polyA signal is located in close proximity to, or adjacent to, an intron such as the SV40 intron.

### RNA Guides

In some embodiments, the CRISPR systems described herein include at least one RNA guide. The architecture of multiple RNA guides is known in the art (see, e.g., International Publication Nos. WO 2014/093622 and WO 2015/070083, the entire contents of each of which are incorporated herein by reference). In some embodiments, the CRISPR systems described herein include multiple RNA guides (e.g., two, three, four, five, six, seven, eight, or more RNA guides). In some embodiments, the RNA guide includes a crRNA and a tracrRNA. In some embodiments, the RNA guide is an engineered construct that comprises a tracrRNA and a crRNA (in a single RNA guide). Sequences for RNA guides from multiple CRISPR systems are known in the art and can be searched using public databases (see, e.g., Grissa et al. (2007) Nucleic Acids Res. 35 (web server issue): W52-7; Grissa et al. (2007) BMC Bioinformatics 8: 172; Grissa et al. (2008) Nucleic Acids Res. 36 (web server issue): W145-8; and Moller and Liang (2017) PeerJ 5: e3788; see also the CRISPR database available at: crispr.i2bc.paris-saclay.fr/crispr/BLAST/CRISPRsBlast.php; and MetaCRAST available at: github.com/molleraj/MetaCRAST).

In some embodiments, the CRISPR systems described herein include at least one RNA guide or a nucleic acid encoding at least one RNA guide. In some embodiments, the RNA guide includes a crRNA. Generally, the crRNAs described herein include a direct repeat sequence and a spacer sequence. In certain embodiments, the crRNA includes, consists essentially of, or consists of a direct repeat sequence linked to a guide sequence or spacer sequence. In some embodiments, the crRNA includes a direct repeat sequence, a spacer sequence, and a direct repeat sequence (DR-spacer-DR), which is typical of precursor crRNA (pre-crRNA) configurations in other CRISPR systems. In some embodiments, the crRNA includes a truncated direct repeat sequence and a spacer sequence, which is typical of processed or mature crRNA. In some embodiments, the crRNA hybridizes with an anti-repeat region of a tracrRNA complementary to the crRNA direct repeat region. In some embodiments, the CRISPR-associated protein forms a complex with the crRNA, and the spacer sequence directs the complex to a sequence-specific binding with the target nucleic acid that is complementary to the spacer sequence. In some embodiments, the CRISPR-associated protein forms a complex with the crRNA and tracrRNA, and the spacer sequence directs the complex to a sequence-specific binding with the target nucleic acid that is complementary to the spacer sequence.

In some embodiments, the CRISPR systems described herein include at least one RNA guide or a nucleic acid encoding at least one RNA guide. In some embodiments, the RNA guide includes a mature crRNA. In some embodiments, the CRISPR systems described herein include a mature crRNA and a tracrRNA. In some embodiments, the CRISPR systems described herein include a pre-crRNA. In some embodiments, the CRISPR systems described herein include a pre-crRNA and a tracrRNA.

Suitably, the Type V-G RNA guide may form a secondary structure such as a stem-loop structure. Suitably, the Type V-G RNA guide may include both a Type V-G crRNA and a Type V-G tracrRNA, either fused into a single RNA molecule or as separate RNA molecules. In some embodiments, a Type V-G crRNA can hybridize with a Type V-G tracrRNA to form a stem-loop structure. An example stem-loop structure of one Type V-G mature crRNA: tracrRNA is shown in FIG. 13. The complementary sections of the crRNA and tracrRNA form the stem. For example, the stem may include at least 8 or at least 9 or at least 10 or at about 11 base pairs.

In some examples, the direct repeat may comprise at least 12 or at least 14 or at least 16 or about 18 nucleotides. The direct repeat can include the nucleic acid sequence kACACC proximal to the spacer, wherein k denotes G or T.

In some embodiments, the CRISPR systems described herein include a plurality of RNA guides (e.g., 2, 3, 4, 5, 10, 15, or more) or a plurality of nucleic acids encoding a plurality of RNA guides.

In some embodiments, the CRISPR system described herein includes an RNA guide or a nucleic acid encoding the RNA guide. In some embodiments, the RNA guide comprises or consists of a direct repeat sequence and a spacer sequence capable of hybridizing (e.g., hybridizes under appropriate conditions) to a target nucleic acid. In some embodiments, the direct repeat sequence includes kACACC (wherein k denotes G or T) proximal to its 3′ end and adjacent to the spacer sequence.

In some embodiments, the RNA guide comprises or consists of a direct repeat sequence and a spacer sequence capable of hybridizing (e.g., hybridizes under appropriate conditions) to a target nucleic acid. In some embodiments, the direct repeat sequence includes kACACC (wherein k denotes G or T) proximal to its 3′ end and adjacent to the spacer sequence.

In some embodiments, the RNA guide comprises a nucleic acid sequence selected from Table 3A, Table 4, or Table 7A-7D. In some embodiments, the RNA guide comprises a nucleic acid sequence selected from one of SEQ ID NOs: 14-26, SEQ ID NO: 28, SEQ ID NOs: 315-506, SEQ ID NOs: 509-756, or SEQ ID NOs: 759-772, or a nucleic acid sequence with at least 70%, at least 75%, at least 80%, at least 85%, at least 90%, at least 95%, or at least 99% sequence identity to one of SEQ ID NOs: 14-26, SEQ ID NO: 28, SEQ ID NOs: 315-506, SEQ ID NOs: 509-756, or SEQ ID NOs: 759-772, or a functional fragment thereof.

In some embodiments, the RNA guide comprises a target-hybridizing sequence selected from Table 7A, or a corresponding RNA sequence, or a combination thereof. In some embodiments, the RNA guide comprises a target-hybridizing sequence selected from one of SEQ ID NOs: 509-523 or a nucleic acid sequence with at least 70%, at least 75%, at least 80%, at least 85%, at least 90%, at least 95%, or at least 99% sequence identity to one of SEQ ID NOs: 509-523, or a corresponding RNA sequence, or a combination thereof, or a functional fragment thereof.

In some embodiments, the RNA guide comprises a scaffold sequence selected from Table 7A, or a corresponding RNA sequence, or a combination thereof. In some embodiments, the RNA guide comprises a scaffold sequence selected from one of SEQ ID NOs: 524-537 or SEQ ID NO: 772 or a nucleic acid sequence with at least 70%, at least 75%, at least 80%, at least 85%, at least 90%, at least 95%, or at least 99% sequence identity to one of SEQ ID NOs: 524-537 or SEQ ID NO: 772, or a corresponding RNA sequence, or a combination thereof, or a functional fragment thereof.

In some embodiments, the RNA guide comprises a DNA sequence selected from Table 7B, or a corresponding RNA sequence, or combination thereof. In some embodiments, the RNA guide comprises a DNA sequence selected from one of SEQ ID NOs: 538-610 or a nucleic acid sequence with at least 70%, at least 75%, at least 80%, at least 85%, at least 90%, at least 95%, or at least 99% sequence identity to one of SEQ ID NOs: 538-610, or a corresponding RNA sequence, or combination thereof, or a functional fragment thereof.

In some embodiments, the RNA guide comprises an RNA sequence selected from Table 7C. In some embodiments, the RNA guide comprises an RNA sequence selected from one of SEQ ID NOs: 611-683 or a nucleic acid sequence with at least 70%, at least 75%, at least 80%, at least 85%, at least 90%, at least 95%, or at least 99% sequence identity to one of SEQ ID NOs: 611-683, or a functional fragment thereof.

In some embodiments, the RNA guide comprises a modified RNA sequence selected from Table 7D. In some embodiments, the RNA guide comprises a modified RNA sequence selected from one of SEQ ID NOs: 684-756 or a nucleic acid sequence with at least 70%, at least 75%, at least 80%, at least 85%, at least 90%, at least 95%, or at least 99% sequence identity to one of SEQ ID NOs: 684-756, or a functional fragment thereof.

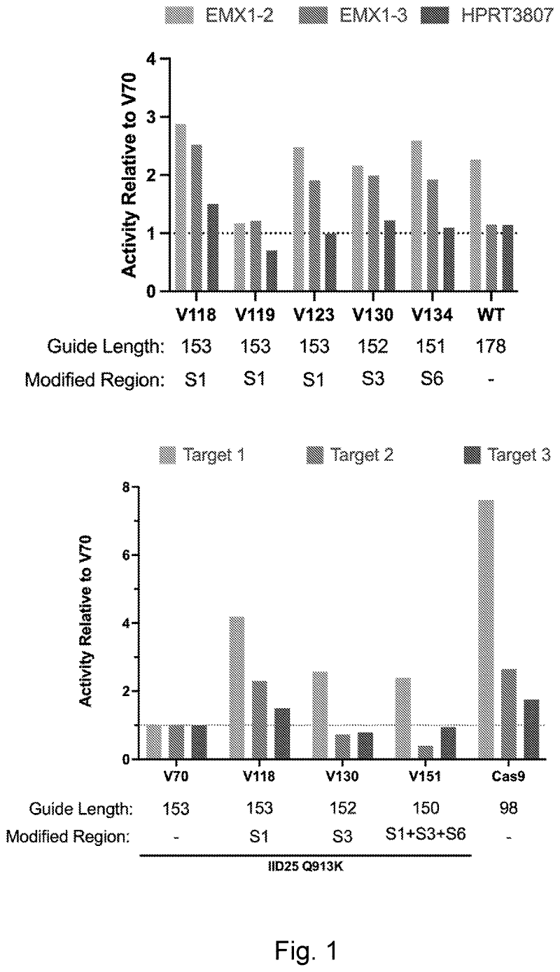

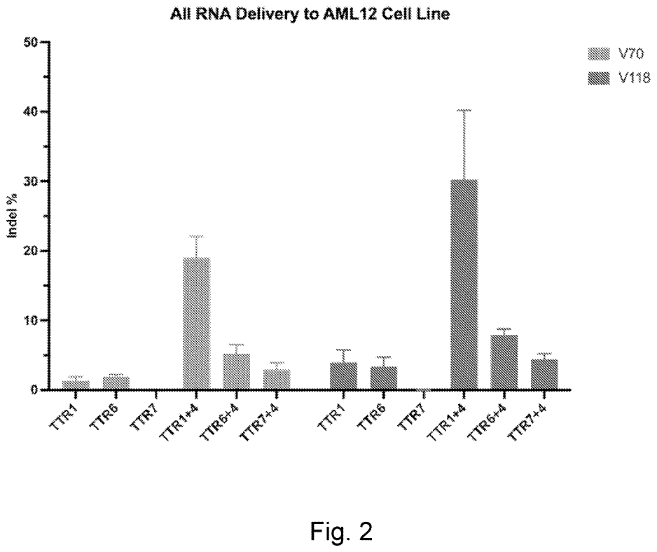

In some embodiments, the RNA guide comprises V118 (see e.g., FIG. 1-2). In some embodiments, the V118 RNA guide comprises the nucleic acid sequence of SEQ ID NO: 526, or a nucleic acid sequence with at least 70%, at least 75%, at least 80%, at least 85%, at least 90%, at least 95%, or at least 99% sequence identity to SEQ ID NO: 526, or a corresponding RNA sequence, or combination thereof, or a functional fragment thereof.

In some embodiments, the V118 RNA guide comprises the DNA sequence of one of SEQ ID NOs: 547, 556, 565, 588, 589, 590, 594, 595, 596, 598, 605, 606, 607, 608, 609, 610 or a DNA sequence with at least 70%, at least 75%, at least 80%, at least 85%, at least 90%, at least 95%, or at least 99% sequence identity to one of SEQ ID NOs: 547, 556, 565, 588, 589, 590, 594, 595, 596, 598, 605, 606, 607, 608, 609, 610, or a corresponding RNA sequence, or combination thereof, or a functional fragment thereof.

In some embodiments, the V118 RNA guide comprises the RNA sequence of one of SEQ ID NOs: 620, 629, 638, 661, 662, 663, 667, 668, 669, 671, 678, 679, 680, 681, 682, 683 or an RNA sequence with at least 70%, at least 75%, at least 80%, at least 85%, at least 90%, at least 95%, or at least 99% sequence identity to one of SEQ ID NOs: 620, 629, 638, 661, 662, 663, 667, 668, 669, 671, 678, 679, 680, 681, 682, 683, or a functional fragment thereof.

In some embodiments, the V118 RNA guide comprises the modified RNA sequence of one of SEQ ID NOs: 693, 702, 711, 734, 735, 736, 740, 741, 742, 744, 751, 752, 753, 754, 755, 756 or a modified RNA sequence with at least 70%, at least 75%, at least 80%, at least 85%, at least 90%, at least 95%, or at least 99% sequence identity to one of SEQ ID NOs: 693, 702, 711, 734, 735, 736, 740, 741, 742, 744, 751, 752, 753, 754, 755, 756, or a functional fragment thereof.

### Multiplexing RNA Guides

Cluster_95538 CRISPR-Cas effector proteins have been demonstrated to employ more than one RNA guide, thus enabling the ability of these effectors, and systems and complexes that include them, to target multiple different nucleic acid targets. In some embodiments, the CRISPR systems described herein include multiple RNA guides (e.g., two, three, four, five, six, seven, eight, nine, ten, fifteen, twenty, thirty, forty, or more RNA guides).

In some embodiments, the CRISPR systems described herein include a single RNA strand or a nucleic acid encoding a single RNA strand, wherein the RNA guides are arranged in tandem.

The single RNA strand can include multiple copies of the same RNA guide, multiple copies of distinct RNA guides, or combinations thereof.

In some embodiments, the Type V-G CRISPR-Cas effector proteins are delivered complexed with multiple RNA guides directed to different target nucleic acids. In some embodiments, the Type V-G CRISPR-Cas effector proteins can be co-delivered with multiple RNA guides, each specific for a different target nucleic acid. Methods of multiplexing using Cas9-IID proteins are described, for example, in U.S. Pat. No. 9,790,490, and EP 3009511, the entire contents of each of which are expressly incorporated herein by reference.

### RNA Guide Modifications

### Spacer Lengths

The spacer length of RNA guides can range from about 15 to 50 nucleotides. In some embodiments, the spacer length of a RNA guide is at least 16 nucleotides, at least 17 nucleotides, at least 18 nucleotides, at least 19 nucleotides, at least 20 nucleotides, at least 21 nucleotides, or at least 22 nucleotides. In some embodiments, the spacer length is from 15 to 17 nucleotides, from 15 to 23 nucleotides, from 15 to 30 nucleotides, from 16 to 22 nucleotides, from 17 to 20 nucleotides, from 20 to 24 nucleotides (e.g., 20, 21, 22, 23, or 24 nucleotides), from 23 to 25 nucleotides (e.g., 23, 24, or 25 nucleotides), from 24 to 27 nucleotides, from 27 to 30 nucleotides, from 30 to 45 nucleotides (e.g., 30, 31, 32, 33, 34, 35, 40, or 45 nucleotides), from 30 or 35 to 40 nucleotides, from 41 to 45 nucleotides, from 45 to 50 nucleotides, or longer. In some embodiments, the spacer length of the RNA guide is at least 16 nucleotides, or is from 16 to 20 nucleotides (e.g., 16, 17, 18, 19, or 20 nucleotides). In some embodiments, the spacer length of the RNA guide is 19 nucleotides.

The RNA guide sequences can be modified in a manner that allows for formation of the CRISPR complex and successful binding to the target, while at the same time not allowing for successful nuclease activity (i.e., without nuclease activity/without causing indels). These modified guide sequences are referred to as “dead guides” or “dead guide sequences.” These dead guides or dead guide sequences may be catalytically inactive or conformationally inactive with regard to nuclease activity. Dead guide sequences are typically shorter than respective guide sequences that result in active RNA cleavage. In some embodiments, dead guides are 5%, 10%, 20%, 30%, 40%, or 50%, shorter than respective RNA guides that have nuclease activity. Dead guide sequences of RNA guides can be from 13 to 15 nucleotides in length (e.g., 13, 14, or 15 nucleotides in length), from 15 to 19 nucleotides in length, or from 17 to 18 nucleotides in length (e.g., 17 nucleotides in length).

Thus, in one aspect, the disclosure provides non-naturally occurring or engineered CRISPR systems including a functional Cas9-IID as described herein, and a RNA guide wherein the RNA guide includes a dead guide sequence whereby the RNA guide is capable of hybridizing to a target sequence such that the CRISPR system is directed to a genomic locus of interest in a cell without detectable cleavage activity. A detailed description of dead guides is described, e.g., in WO 2016094872, which is incorporated herein by reference in its entirety.

### Inducible Guides

RNA guides can be generated as components of inducible systems. The inducible nature of the systems allows for spatiotemporal control of gene editing or gene expression. In some embodiments, the stimuli for the inducible systems include, e.g., electromagnetic radiation, sound energy, chemical energy, and/or thermal energy.

In some embodiments, the transcription of RNA guides can be modulated by inducible promoters, e.g., tetracycline or doxycycline controlled transcriptional activation (Tet-On and Tet-Off expression systems), hormone inducible gene expression systems (e.g., ecdysone inducible gene expression systems), and arabinose-inducible gene expression systems. Other examples of inducible systems include, e.g., small molecule two-hybrid transcription activations systems (FKBP, ABA, etc.), light inducible systems (Phytochrome, LOV domains, or cryptochrome), or Light Inducible Transcriptional Effector (LITE). These inducible systems are described, e.g., in WO 2016205764 and U.S. Pat. No. 8,795,965, both of which are incorporated herein by reference in their entirety.

### Chemical Modifications

Chemical modifications can be applied to the RNA guide's phosphate backbone, sugar, and/or base. Backbone modifications such as phosphorothioates modify the charge on the phosphate backbone and aid in the delivery and nuclease resistance of the oligonucleotide (see, e.g., Eckstein, “Phosphorothioates, essential components of therapeutic oligonucleotides,” Nucl. Acid Ther., 24 (2014), pp. 374-387); modifications of sugars, such as 2′-O-methyl (2′-OMe), 2′-F, and locked nucleic acid (LNA), enhance both base pairing and nuclease resistance (see, e.g., Allerson et al. “Fully 2′-modified oligonucleotide duplexes with improved in vitro potency and stability compared to unmodified small interfering RNA,” J Med. Chem., 48.4 (2005): 901-904). Chemically modified bases such as 2-thiouridine or N6-methyladenosine, among others, can allow for either stronger or weaker base pairing (see, e.g., Bramsen et al., “Development of therapeutic-grade small interfering RNAs by chemical engineering,” Front. Genet. 2012 Aug. 20; 3: 154). Additionally, RNA is amenable to both 5′ and 3′ end conjugations with a variety of functional moieties including fluorescent dyes, polyethylene glycol, or proteins.

A wide variety of modifications can be applied to chemically synthesized RNA guide molecules. For example, modifying an oligonucleotide with a 2′-OMe to improve nuclease resistance can change the binding energy of Watson-Crick base pairing. Furthermore, a 2′-OMe modification can affect how the oligonucleotide interacts with transfection reagents, proteins or any other molecules in the cell. The effects of these modifications can be determined by empirical testing.

In some embodiments, the RNA guide includes one or more phosphorothioate modifications. In some embodiments, the RNA guide includes one or more locked nucleic acids for the purpose of enhancing base pairing and/or increasing nuclease resistance.

A summary of these chemical modifications can be found, e.g., in Kelley et al., “Versatility of chemically synthesized guide RNAs for CRISPR-Cas9 genome editing,” J. Biotechnol. 2016 Sep. 10; 233:74-83; WO 2016205764; and U.S. Pat. No. 8,795,965 B2; each which is incorporated by reference in its entirety.

### Sequence Modifications

The sequences and the lengths of the RNA guides (e.g., tracrRNAs and crRNAs) described herein can be optimized. In some embodiments, the optimized length of RNA guides can be determined by identifying the processed form of tracrRNA and/or crRNA, or by empirical length studies for RNA guides, tracrRNAs, crRNAs, and the tracrRNA tetraloops.

The RNA guides can also include one or more aptamer sequences. Aptamers are oligonucleotide or peptide molecules that can bind to a specific target molecule. The aptamers can be specific to gene effectors, gene activators, or gene repressors. In some embodiments, the aptamers can be specific to a protein, which in turn is specific to and recruits/binds to specific gene effectors, gene activators, or gene repressors. The effectors, activators, or repressors can be present in the form of fusion proteins. In some embodiments, the RNA guide has two or more aptamer sequences that are specific to the same adaptor proteins. In some embodiments, the two or more aptamer sequences are specific to different adaptor proteins. The adaptor proteins can include, e.g., MS2, PP7, Q3, F2, GA, fr, JP501, M12, R17, BZ13, JP34, JP500, KU1, M11, MX1, TW18, VK, SP, FI, ID2, NL95, TW19, AP205, φCb5, φCb8r, φCb12r, φCb23r, 7s, and PRR1. Accordingly, in some embodiments, the aptamer is selected from binding proteins specifically binding any one of the adaptor proteins as described herein. In some embodiments, the aptamer sequence is a MS2 binding loop, QBeta binding loop, or PP7 binding loop. A detailed description of aptamers can be found, e.g., in Nowak et al., “Guide RNA engineering for versatile Cas9 functionality,” Nucl. Acid. Res., 2016 Nov. 16; 44(20):9555-9564; and WO 2016205764, which are incorporated herein by reference in their entirety.

### Guide: Target Sequence Matching Requirements

In classic CRISPR systems, the degree of complementarity between a guide sequence and its corresponding target sequence can be about 50%, 60%, 75%, 80%, 85%, 90%, 95%, 97.5%, 99%, or 100%. In some embodiments, the degree of complementarity is 100%. The RNA guides can be about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length.

To reduce off-target interactions, e.g., to reduce the guide interacting with a target sequence having low complementarity, mutations can be introduced to the CRISPR systems so that the CRISPR systems can distinguish between target and off-target sequences that have greater than 80%, 85%, 90%, or 95% complementarity. In some embodiments, the degree of complementarity is from 80% to 95%, e.g., about 83%, 84%, 85%, 86%, 87%, 88%, 89%, 90%, 91%, 92%, 93%, 94%, or 95% (for example, distinguishing between a target having 18 nucleotides from an off-target of 18 nucleotides having 1, 2, or 3 mismatches). Accordingly, in some embodiments, the degree of complementarity between a guide sequence and its corresponding target sequence is greater than 94.5%, 95%, 95.5%, 96%, 96.5%, 97%, 97.5%, 98%, 98.5%, 99%, 99.5%, or 99.9%. In some embodiments, the degree of complementarity is 100%.

Some embodiments of the technology described herein can be defined according to any of the following numbered paragraphs:

Embodiment 1. A Cas9-IID system comprising: a) a Cas9-IID proteins effector polypeptide, or one or more nucleotide sequences encoding the protein effector polypeptide, wherein the protein effector polypeptide comprises an amino acid sequence SEQ ID NO: 9, or comprises a variant of a protein effector comprising an amino acid sequence having at least 60%, 65%, 70%, 75%, 80%, 85%, 90%, or 95% identity to an amino acid sequence selected from the group consisting of SEQ ID NO: 9, or comprises the amino acid sequence of a naturally-occurring protein effector having at least 30% identity to an amino acid sequence selected from the group consisting of SEQ ID NO:9, and b) one or more engineered guide RNAs comprising a guide sequence, wherein the one or more guide RNAs is designed to form a complex with the protein effector polypeptide and wherein the one or more guide RNAs comprises a guide sequence (also referred to as guide ribonucleic acid sequence) designed to hybridize with one or more target nucleic acid molecules, wherein the guide RNA and the protein effector polypeptide do not naturally occur together.

Embodiment 2. The Cas9-IID system of embodiment 1, wherein the amino acid sequence of the Cas9-IID protein comprises at least one, at least two, at least three, at least four, at least five, at least six, at least seven, at least eight, or at least nine mutation(s) at position(s) 7, 55, 56, 57, 58, 59, 60, 61, 62, 63, 64, 65, 66, 624, 858, 859, 860, 861, 862, 863, 864, 865, 866, 867, 868, 896, 897, 898, 899, 900, 901, 902, 903, and/or 904 of the amino acid sequence of SEQ ID NO: 9.

Embodiment 3. An engineered, non-naturally occurring Cas9-IID system comprising one or more nucleic acid constructs comprising: a) a polynucleotide sequence encoding a protein effector polypeptide; wherein the protein effector polypeptide comprises an amino acid sequence selected from the group comprising SEQ ID NO:9, or comprises a variant of a protein effector comprising an amino acid sequence having at least 60%, 65%, 70%, 75%, 80%, 85%, 90%, or 95% identity to an amino acid sequence selected from the group consisting of SEQ ID NO:9, or comprises the amino acid sequence of a naturally-occurring protein effector having at least 50% identity to an amino acid sequence selected from the group consisting of SEQ ID NO:9, and b) a polynucleotide sequence encoding a guide RNA, wherein the guide RNA is designed to form a complex with the protein effector polypeptide and wherein the guide RNA comprises a guide sequence designed to hybridize with one or more target nucleic acid molecules, wherein the guide RNA and the protein effector polypeptide do not naturally occur together.

Embodiment 4. An engineered, non-naturally occurring Cas9-IID system according to embodiment 3, wherein the polynucleotide sequence encoding the protein polypeptide and the polynucleotide sequence encoding a guide RNA are located on the same or different nucleic acid constructs of the system, wherein when transcribed, the one or more guide RNAs forms one or more complexes with the Protein effector polypeptide, and wherein the one or more guide RNAs hybridizes to the one or more target nucleic acid molecules, resulting in cleavage of the target nucleic acid molecule.

Embodiment 5. An engineered, non-naturally occurring Cas9-IID system comprising one or more nucleic acid constructs comprising: a) a polynucleotide sequence encoding a Cas9-IID protein effector polypeptide; wherein the protein effector polypeptide comprises an amino acid sequence selected from the group comprising SEQ ID NO: 1, SEQ ID NO:2, SEQ ID NO:3, SEQ ID NO:4, SEQ ID NO:5, SEQ ID NO:6, SEQ ID NO:7, SEQ ID NO:8, SEQ ID NO:10, SEQ ID NO: 11 and SEQ ID NO:13, or comprises a variant of a Cas9-IID protein effector comprising an amino acid sequence having at least 60%, 65%, 70%, 75%, 80%, 85%, 90%, or 95% identity to an amino acid sequence selected from the group consisting of SEQ ID NO: 1, SEQ ID NO:2, SEQ ID NO:3, SEQ ID NO:4, SEQ ID NO:5, SEQ ID NO:6, SEQ ID NO:7, SEQ ID NO:8, SEQ ID NO:10, SEQ ID NO: 11 and SEQ ID NO:13; or comprises the amino acid sequence of a naturally-occurring Cas9-IID protein effector having at least 50% identity to an amino acid sequence selected from the group consisting of SEQ ID NO: 1, SEQ ID NO:2, SEQ ID NO:3, SEQ ID NO:4, SEQ ID NO:5, SEQ ID NO:6, SEQ ID NO:7, SEQ ID NO:8, SEQ ID NO:10, SEQ ID NO: 11 and SEQ ID NO:13, and b) a polynucleotide sequence encoding a guide RNA, wherein the guide RNA is designed to form a complex with the protein effector polypeptide and wherein the guide RNA comprises a guide sequence designed to hybridize with one or more target nucleic acid molecules, wherein the guide RNA and the protein effector polypeptide do not naturally occur together, and wherein and wherein the amino acid sequence of the Cas9-IID protein comprises at least one, at least two, at least three, at least four, at least five, at least six, at least seven, at least eight, or at least nine mutation(s) at position(s) 7, 45, 46, 47, 48, 49, 50, 51, 52, 53, 54, 55, 56, 558, 842, 843, 844, 845, 846, 847, 848, 849, 850, 851, 852, 853, 854, 908, 910, 911, 912, 913, 914, 915, 916, 917, 918, 919, 920, and/or 921.

Embodiment 6. An engineered, non-naturally occurring Cas9-IID system according to embodiment 5, wherein the polynucleotide sequence encoding the protein polypeptide and the polynucleotide sequence encoding a guide RNA are located on the same or different nucleic acid constructs of the system, wherein when transcribed, the one or more guide RNAs forms one or more complexes with the Protein effector polypeptide, and wherein the one or more guide RNAs hybridizes to the one or more target nucleic acid molecules, resulting in cleavage of the target nucleic acid molecule.

Embodiment 7. An engineered, non-naturally occurring Cas9-IID system comprising one or more nucleic acid constructs comprising: a) a polynucleotide sequence encoding a Cas9-IID protein effector polypeptide; wherein the protein effector polypeptide comprises an amino acid sequence selected from the group comprising SEQ ID NO: 12, or comprises a variant of a Cas9-IID protein effector comprising an amino acid sequence having at least 60%, 65%, 70%, 75%, 80%, 85%, 90%, or 95% identity to an amino acid sequence selected from the group consisting of SEQ ID NO: 12, or comprises the amino acid sequence of a naturally-occurring Cas9-IID protein effector having at least 50% identity to an amino acid sequence selected from the group consisting of SEQ ID NO: 12, and b) a polynucleotide sequence encoding a guide RNA, wherein the guide RNA is designed to form a complex with the protein effector polypeptide and wherein the guide RNA comprises a guide sequence designed to hybridize with one or more target nucleic acid molecules, wherein the guide RNA and the protein effector polypeptide do not naturally occur together, and wherein and wherein the amino acid sequence of the Cas9-IID protein comprises at least one, at least two, at least three, at least four, at least five, at least six, at least seven, at least eight, or at least nine mutation(s) at position(s) 7, 45, 46, 47, 48, 49, 50, 51, 52, 53, 54, 55, 56, 558, 843, 844, 845, 846, 847, 848, 849, 850, 851, 852, 853, 854, 855, 909, 910, 911, 912, 913, 914, 915, 916, 917, 918, 919, 920, 921, and/or 922 of the amino acid sequence of SEQ ID NO: 12.

Embodiment 8. An engineered, non-naturally occurring Cas9-IID system according to embodiment 5, wherein the polynucleotide sequence encoding the protein polypeptide and the polynucleotide sequence encoding a guide RNA are located on the same or different nucleic acid constructs of the system, wherein when transcribed, the one or more guide RNAs forms one or more complexes with the Protein effector polypeptide, and wherein the one or more guide RNAs hybridizes to the one or more target nucleic acid molecules, resulting in cleavage of the target nucleic acid molecule.

Embodiment 9. An engineered, non-naturally occurring Cas9-IID system comprising one or more nucleic acid constructs comprising: a) a polynucleotide sequence encoding a Cas9-IID protein effector polypeptide; wherein the protein effector polypeptide comprises an amino acid sequence selected from the group comprising SEQ ID NO: 1, or comprises a variant of a Cas9-IID protein effector comprising an amino acid sequence having at least 60%, 65%, 70%, 75%, 80%, 85%, 90%, or 95% identity to an amino acid sequence selected from the group consisting of SEQ ID NO: 1, or comprises the amino acid sequence of a naturally-occurring Cas9-IID protein effector having at least 50% identity to an amino acid sequence selected from the group consisting of SEQ ID NO: 1, and b) a polynucleotide sequence encoding a guide RNA, wherein the guide RNA is designed to form a complex with the protein effector polypeptide and wherein the guide RNA comprises a guide sequence designed to hybridize with one or more target nucleic acid molecules, wherein the guide RNA and the protein effector polypeptide do not naturally occur together, and wherein and wherein the amino acid sequence of the Cas9-IID protein comprises at least one, at least two, at least three, at least four, at least five, at least six, at least seven, at least eight, or at least nine mutation(s) at position(s) 13, 19, 20, 40, 69, 70, 71, 89, 105, 106, 131, 153, 180, 185, 215, 221, 239, 302, 366, 367, 370, 372, 376, 387 410, 458, 469, 473, 495, 537, 538, 571, 598, 609, 610, 611, 657, 700, 736, 737, 786, 800, 821, 827, 828, 843, 866, 873, 901, 913, 928, and 930 of the amino acid sequence of SEQ ID NO: 1.

Embodiment 10. The Cas9-IID system of any of embodiments 1-9, further comprising a target nucleic acid molecule.

Embodiment 11. The Cas9-IID system of embodiment 10, wherein the target nucleic acid molecule is a prokaryotic target nucleic acid molecule.

Embodiment 12. The Cas9-IID system of embodiment 10, wherein the target nucleic acid molecule is a eukaryotic target nucleic acid molecule.

Embodiment 13. The Cas9-IID system of embodiment 10, wherein the target nucleic acid molecule is within a cell.

Embodiment 14. The Cas9-IID system of embodiment 13, wherein the cell is a prokaryotic cell.

Embodiment 15. The Cas9-IID system of embodiment 13, wherein the cell is a eukaryotic cell.

Embodiment 16. The Cas9-IID system of embodiment 15, wherein the nucleotide sequence encoding the Cas9-IID protein effector polypeptide is codon optimized for expression in a eukaryotic cell.

Embodiment 17. The Cas9-IID system of any one of embodiments 1-16, further comprising one or more guide RNAs.

Embodiment 18. The Cas9-IID system of any of embodiment 1-17, wherein the polynucleotide sequence encoding a guide RNA encodes one or more guide RNAs.

Embodiment 19. The Cas9-IID system of any one of embodiments 1-18, wherein said engineered guide ribonucleic acid structure (e.g., guide RNA) comprises a single ribonucleic acid polynucleotide comprising said guide ribonucleic acid sequence and a tracr sequence (e.g., tracr ribonucleic acid sequence).

Embodiment 20. The Cas9-IID system of any one of embodiments 1-19, wherein said guide ribonucleic acid sequence is complementary to a prokaryotic, bacterial, archaeal, eukaryotic, fungal, plant, mammalian, or human genomic sequence.

Embodiment 21. The Cas9-IID system of any one of embodiments 1-20, wherein said guide ribonucleic acid sequence is 15-25 nucleotides in length.

Embodiment 22. The Cas9-IID system of any one of embodiments 1-21, wherein said endonuclease comprises one or more nuclear localization sequences (NLSs) proximal to an N- or C-terminus of said Cas9-IID nuclease.

Embodiment 23. The Cas9-IID of any one of embodiments 1-22, further comprising a single- or double-stranded DNA repair template comprising from 5′ to 3′: a first homology arm comprising a sequence of at least 20 nucleotides 5′ to said target deoxyribonucleic acid sequence, a synthetic DNA sequence of at least 10 nucleotides, and a second homology arm comprising a sequence of at least 20 nucleotides 3′ to said target sequence.

Embodiment 24. The Cas9-IID system of embodiment 23, wherein said first or second homology arm comprises a sequence of at least 40, 80, 120, 150, 200, 300, 500, or 1,000 nucleotides.

Embodiment 25. The Cas9-IID system of any one of embodiments 1-24, wherein said system further comprises a source of Mg2+.

Embodiment 26. The Cas9-IID system of any one of embodiments 1-25 wherein said Cas9-IID and said tracr ribonucleic acid sequence are derived from distinct bacterial species within a same phylum.

Embodiment 27. The Cas9-IID system of embodiment 26, wherein said guide RNA structure further comprises a second stem and a second loop, wherein the second stem comprises at least 5 pairs of ribonucleotides.

Embodiment 28. The Cas9-IID system of embodiment 27, wherein said guide RNA structure further comprises an RNA structure comprising at least two hairpins.

Embodiment 29. A deoxyribonucleic acid polynucleotide encoding the engineered guide ribonucleic acid polynucleotide of any one of embodiments 1-28 or 65.

Embodiment 30. A method for binding, cleaving, marking, or modifying a double-stranded deoxyribonucleic acid polynucleotide, comprising: (a) contacting said double-stranded deoxyribonucleic acid polynucleotide with a Cas9-IID endonuclease in complex with an engineered guide ribonucleic acid structure configured to bind to said Cas9-IID endonuclease and said double-stranded deoxyribonucleic acid polynucleotide; (b) wherein said double-stranded deoxyribonucleic acid polynucleotide comprises a protospacer adjacent motif (PAM); and wherein the Cas9-IID endonuclease is selected from the group comprising SEQ ID NO: 1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO: 12, and SEQ ID NO:13 with at least one mutation at position 7, 45, 46, 47, 48, 49, 50, 51, 52, 53, 54, 55, 56, 558, 842, 843, 844, 845, 846, 847, 848, 849, 850, 851, 852, 853, 854, 908, 910, 911, 912, 913, 914, 915, 916, 917, 918, 919, 920, and/or 921.

Embodiment 31. The method of embodiment 30, wherein said Cas9-IID endonuclease cleaves said double-stranded deoxyribonucleic acid polynucleotide, wherein said PAM comprises NGG, NACC, NVC, NRGM, NAC, NVCCC, NAV, NVC, or NAC.

Embodiment 32. The method of embodiment 30 or embodiment 31, wherein said Cas9-IID endonuclease cleaves said double-stranded deoxyribonucleic acid polynucleotide 6-8 nucleotides or 7 nucleotides from said PAM.

Embodiment 33. A method of modifying a target nucleic acid locus, said method comprising delivering to said target nucleic acid locus said engineered Cas9-IID system of any one of embodiments 1-29, wherein said Cas9-IID is configured to form a complex with said engineered guide ribonucleic acid structure, and wherein said complex is configured such that upon binding of said complex to said target nucleic acid locus, said complex modifies said target nucleic locus.

Embodiment 34. The method of embodiment 33, wherein modifying said target nucleic acid locus comprises binding, nicking, cleaving, or marking said target nucleic acid locus.

Embodiment 35. The method of embodiment 33 or embodiment 34, wherein said target nucleic acid locus comprises deoxyribonucleic acid (DNA) or ribonucleic acid (RNA).

Embodiment 36. The method of any one of embodiments 33-35, wherein delivering said engineered Cas9-IID system to said target nucleic acid locus comprises delivering a translated polypeptide.

Embodiment 37. A Cas9-IID system comprising: a) a Cas9-IID proteins effector polypeptide, or one or more nucleotide sequences encoding the protein effector polypeptide, wherein the protein effector polypeptide comprises an amino acid sequence SEQ ID NO:1, or comprises a variant of a protein effector comprising an amino acid sequence having at least 60%, 65%, 70%, 75%, 80%, 85%, 90%, or 95% identity to an amino acid sequence selected from the group consisting of SEQ ID NO:1, or comprises the amino acid sequence of a naturally-occurring protein effector having at least 70% identity to an amino acid sequence selected from the group consisting of SEQ ID NO: 1, and b) one or more engineered guide RNA, wherein said engineered guide RNA comprises a secondary structure (i.e., structure A) as shown in FIG. 3, wherein N20 is a spacer sequence of 15-25 nucleotides at the 5′-end; wherein S1, S2, S3, S4, S5 and S6 comprise independently 2-20 nucleotide base-pairs; and wherein the engineered guide RNA is less than 155 nucleotides and comprising at least one modification.

Embodiment 38. The Cas9-IID system of embodiment 37, wherein said modification comprises deletion of one to four base-pairs in the S1.

Embodiment 39. The Cas9-IID system of embodiment 37, wherein said modification comprises deletion of S5 and/or S6.

Embodiment 40. The Cas9-IID system of embodiment 37, wherein said modification comprises deletion of at least three nucleotides at the 3′-end.

Embodiment 41. The Cas9-IID system of embodiment 37, wherein said modification comprises deletion of two nucleotide base-pairs in the S1, deletion of 5 nucleotides at the 3′-end, and deletion of the U in the S3 and deletion of S5 and S6.

Embodiment 42. The Cas9-IID system of embodiment 37, wherein the guide ribonucleic acid sequence is 20 or 21 nucleotides in length.

Embodiment 43. The Cas9-IID system of embodiment 37, wherein the deleted nucleotides are replaced with a linker (e.g., a loop portion or pin-loop).

Embodiment 44. The Cas9-IID system of embodiment 43, wherein the linker is single-stranded nucleic acid comprising from about from about 4 nucleotides to about 15 nucleotides.

Embodiment 45. The Cas9-IID system of embodiment 43, wherein a loop portion (pin-loop) of the 5′-stem-loop comprises from 4, 5 or 6 nucleotides.

Embodiment 46. The Cas9-IID system of embodiment 43, wherein a loop portion of the 5′-stem-loop comprises the nucleotide sequence GAAA or GAAAA.

Embodiment 47. The Cas9-IID system of embodiment 43, wherein a loop region (pin-loop) of the 5′-stem-loop comprises a nucleic acid modification.

Embodiment 48. The Cas9-IID system of embodiment 37, wherein said nucleic acid modification is a modified nucleotide selected from the group consisting of 2′-O-methyl (2′-OMe) nucleotides, 2′-fluoro (2′-F) nucleotides, bridged nucleic acid (BNA) nucleotides (e.g., 2′-0,4′-C-methylene (locked nucleic acid, LNA) nucleotides, 2′-0,4′-C-ethylene (locked nucleic acid, ENA) nucleotides, 5′-methyl-BNA, cEt BNA, cMOE BNA, oxy amino BNA and vinyl-carbo BNA), anhydrohexitol (1,5-anhydrohexitol nucleic acid, HNA) nucleotides, cyclohexene (Cyclohexene nucleic acid, CeNA) nucleotides, 2′-methoxyethyl (2′-MOE) nucleotides, 2′-O-allyl nucleotides, 2′-C-allyl ribose nucleotides, 2′-O—N-methylacetamido (2′-O-NMA) nucleotides, a 2′-O-dimethylaminoethoxyethyl (2′-O-DMAEOE) nucleotides, 2′-O-aminopropyl (2′-O-AP) nucleotides, 2′-F arabinose (2′-ara-F) nucleotides, threose (Threose nucleic acid, TNA) nucleotides, and acyclic nucleotides (e.g., unlocked nucleic acids (UNA) and 2,3-dihydroxylpropyl (glycol nucleic acid, GNA)); a modified internucleoside linkage; a non-natural or modified nucleobase; or a combination thereof.

Embodiment 49. The Cas9-IID system of embodiment 37, wherein said nucleic acid modification is 2′-O-methyl (2′-OMe).

Embodiment 50. The Cas9-IID system of embodiment 37, wherein said nucleic acid modification is 2′-fluoro modified nucleotide.

Embodiment 51. The Cas9-IID system of embodiment 37, wherein said nucleic acid modification is non-natural or modified nucleobase.

Embodiment 52. The Cas9-IID system of embodiment 37, wherein modification comprises at least one (e.g., 1, 2, 3, 4, 5, or more) modified internucleoside linkage (e.g., a modified internucleoside linkage selected independently from the group consisting of phosphorothioates (R, S, or racemic), phosphorodithioates, methylenemethylimino (MMI, 3′-CH2-N(CH3)-O-5′), phosphotriesters, alkylphosphonates (e.g., methylphosphonates), phosphoramidate, methylenemethylimino (—CH2-N(CH3)-O—CH2-), thiodiester (—O—C(O)—S—), thionocarbamate (—O—C(O)(NH)—S—), siloxane (—O—Si(H)2-O— and dialkylsiloxane), N,N′-dimethylhydrazine (—CH2-N(CH3)-N(CH3)-), amide-3 (3′-CH2-C(═O)—N(H)-5′), amide-4 (3′-CH2-N(H)—C(═O)-5′)), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′-S—CH2-O-5′), formacetal (3′-O—CH2-O-5′), oxime, methyleneimino, methykenecarbonylamino, methylenehydrazo, methylenedimethylhydrazo, methyleneoxymethylimino, ethers (C3′-O—C5′), thioethers (C3′-S—C5′), thioacetamido (C3′-N(H)—C(═O)—CH2-S—C5′, C3′-O—P(O)—O—SS—C5′), C3′-CH2-NH—NH—C5′, 3′-NHP(O)(OCH3)-O-5′, 3′—NHP(O)(OCH3)-O-5′), 2′->5′ internucleoside linkages, 2′->3′ internucleoside linkages, 3′->3′ internucleoside linkages, and 5′->5′ internucleoside linkages, optionally the modified internucleoside linkage is phosphorothioate, imidp or MMI, more preferably the modified internucleoside linkage is phosphorothioate (PS).

Embodiment 53. The Cas9-IID system of embodiment 37, wherein modification comprises a duplex stabilizing modification, optionally the duplex stabilizing modification is 2′-F nucleotide, 2′-OMe nucleotide, 2′-methoxyethyl nucleotide, 2,6-diaminopurine nucleotide, 5-methyl cytidine, N4-ethyl cytidine, 5-propynyl cytidine, 5-propynyl uridine, 5-hydroxybutynl-2′-deoxyuridine, 8-aza-7-deazaguanosine, a locked nucleic acid (LNA), and/or covalent cross-linking of two strands of the duplex.

Embodiment 54. The Cas9-IID system of embodiment 37, wherein the modification is at the 3′ end of the guide RNA and comprises any one of: (i) a modification of any one or more of the last 7, 6, 5, 4, 3, 2, or 1 nucleotides; (ii) one modified nucleotide; (iii) two modified nucleotides; (iv) three modified nucleotides; (v) four modified nucleotides; (vi) five modified nucleotides; (vii) six modified nucleotides; or (viii) seven modified nucleotides.

Embodiment 55. The Cas9-IID system of embodiment 37, wherein the modification is at the 3′ end of the guide RNA and comprises any one of: (i) a modified internucleoside linkage (e.g., PS, imidp or MMI linkage) between nucleotides; (ii) a 2′-OMe nucleotide; (iii) a 2′-O-MOE nucleotide; (iv) a 2′-F nucleotide; (v) a LNA nucleotide (vi) a 3′->3′ linkage between nucleotides; (vii) an inverted abasic nucleotide; and (viii) a combination of one or more of (i)-(vii).

Embodiment 56. The Cas9-IID system of embodiment 37, wherein the modification is at the 3′ end of the guide RNA and comprises any one of: (i) a modified internucleoside linkage (e.g., PS, imidp or MMI linkage) between the last and second to last nucleotides; (ii) a modified internucleoside linkage (e.g., PS, imidp or MMI linkage) between the last and second to last nucleotide and a PS linkage between the second and third to last nucleotides; (iii) modified internucleoside linkages (e.g., PS and/or MMI linkage) between any one or more of the last four nucleotides; (iv) modified internucleoside linkages (e.g., PS and/or MMI linkage) between any one or more of the last five nucleotides; or (v) modified internucleoside linkages (e.g., PS and/or MMI linkage) between any one or more of the last 2, 3, 4, 5, 6, 7, 8, 9, or 10 nucleotides.

Embodiment 57. The Cas9-IID system of embodiment 37, wherein the modification is at the 3′ end of the guide RNA and comprises any one of: (i) a modification of one or more of the last 1-7 nucleotides, wherein the modification is a modified internucleoside linkages (e.g., PS and/or MMI linkage), inverted abasic nucleotide, a 3′->3′ internucleoside linkage, 2′-OMe, 2′-O-MOE, 2′-F, LNA, or a combination thereof; (ii) a modification to the last nucleotide with 2′-OMe, 2′-O-MOE, 2′-F, LNA, or combinations thereof, and an optional one or two modified internucleoside linkages (e.g., PS and/or MMI linkage) to the next nucleotide; (iii) a modification to the last and/or second to last nucleotide with 2′-OMe, 2′-O-MOE, 2′-F, LNA, or combinations thereof, and optionally one or more modified internucleoside linkages (e.g., PS and/or MMI linkage); (iv) a modification to the last, second to last, and/or third to last nucleotides with 2′-OMe, 2′-O-moe, 2′-F, or combinations thereof, and optionally one or more modified internucleoside linkages (e.g., PS and/or MMI linkage); (v) a modification to the last, second to last, third to last, and/or fourth to last nucleotides with 2′-OMe, 2′-O-MOE, 2′-F, LNA or combinations thereof, and optionally one or more modified internucleoside linkages (e.g., PS and/or MMI linkage); or (vi) a modification to the last, second to last, third to last, fourth to last, and/or fifth to last nucleotides with 2′-OMe, 2′-O-MOE, 2′-F, LNA, or combinations thereof, and optionally one or more modified internucleoside linkages (e.g., PS and/or MMI linkage).

Embodiment 58. The Cas9-IID system of embodiment 37, wherein the modification is at the 3′ end of the guide RNA and comprises any one of: (i) a 2′-OMe modified nucleotide at the last position, three consecutive 2′-O-MOE modified nucleotides immediately 5′ to the 2′-OMe modified nucleotide, and three consecutive PS linkages between the last three nucleotides; (ii) five consecutive 2′-OMe modified nucleotides from the 3′ end of the 3′ terminus, and three PS linkages between the last three nucleotides; (iii) an inverted abasic modified nucleotide at the last position; (iv) an inverted abasic modified nucleotide at the last position, and three consecutive 2′-OMe modified nucleotides at the last three positions (v) 15 consecutive 2′-OMe modified nucleotides from the 3′ end, five consecutive 2′-F modified nucleotides immediately 5′ to the 2′-OMe modified nucleotides, and three PS linkages between the last three nucleotides; (vi) alternating 2′-OMe modified nucleotides and 2′-F modified nucleotides at the last 20 nucleotides, and three PS linkages between the last three nucleotides; (vii) two or three consecutive 2′-OMe modified nucleotides, and three PS linkages between the last three nucleotides; (viii) one PS linkage between the last and next to last nucleotides; and (ix) 15 or 20 consecutive 2′-OMe modified nucleotides, and three PS linkages between the last three nucleotides.

Embodiment 59. The Cas9-IID system of embodiment 37, wherein the modification is at the 5′ end of the guide RNA and comprises any one of: (i) one modified nucleotide; (ii) two modified nucleotides; (iii) three modified nucleotides; (iv) four modified nucleotides; (v) five modified nucleotides; (vi) six modified nucleotides; and (vii) seven modified nucleotides.

Embodiment 60. The Cas9-IID system of embodiment 37, wherein the modification is at the 5′ end of the guide RNA and comprises any one or more modification of between 1 and 7, between 1 and 5, between 1 and 4, between 1 and 3, or between 1 and 2 nucleotides.

Embodiment 61. The Cas9-IID system of embodiment 37, wherein the 5′ end modification of the guide RNA and comprises any one or more of: (i) a modified internucleoside linkage (e.g., a phosphorothioate and/or MMI linkage) between nucleotides; (ii) a 2′-OMe nucleotide; (iii) a 2′-O-MOE nucleotide; (iv) a 2′-F nucleotide; (v) a LNA; (vi) a 3′->3′ linkage; (vii) an inverted abasic modified nucleotide; (viii) a deoxyribonucleotide; (ix) an inosine; and (x) combinations of one or more of (i)-(ix).

Embodiment 62. The Cas9-IID system of embodiment 37, wherein the modification is at the 5′ end of the guide RNA and comprises: (i) 1, 2, 3, 4, 5, 6, and/or 7 modified internucleoside (e.g., a phosphorothioate and/or MMI) linkages between nucleotides; or (ii) about 1-2, 1-3, 1-4, 1-5, 1-6, or 1-7 modified internucleoside (e.g., a phosphorothioate and/or MMI) linkages between nucleotides.

Embodiment 63. The Cas9-IID system of embodiment 37, wherein the modification is at the 5′ end of the guide RNA and comprises: (i) one modified internucleoside (e.g., a phosphorothioate and/or MMI) linkage, and the linkage is between nucleotides 1 and 2; (ii) two modified internucleoside (e.g., a phosphorothioate and/or MMI) linkages, and the linkages are between nucleotides 1 and 2, and 2 and 3; (iii) modified internucleoside (e.g., a phosphorothioate and/or MMI) linkages between any one or more of nucleotides 1 and 2, 2 and 3, and 3 and 4; (iv) modified internucleoside (e.g., a phosphorothioate and/or MMI) linkages between any one or more of nucleotides 1 and 2, 2 and 3, 3 and 4, and 4 and 5; (v) modified internucleoside (e.g., a phosphorothioate and/or MMI) linkages between any one or more of nucleotides 1 and 2, 2 and 3, 3 and 4, 4 and 5, and 5 and 6; (vi) modified internucleoside (e.g., a phosphorothioate and/or MMI) linkages between any one or more of nucleotides 1 and 2, 2 and 3, 3 and 4, 4 and 5, 5 and 6, and 6 and 7; or (vii) modified internucleoside (e.g., a phosphorothioate and/or MMI) linkages between any one or more of nucleotides 1 and 2, 2 and 3, 3 and 4, 4 and 5, 5 and 6, 6 and 7, and 7 and 8.

Embodiment 64. The Cas9-IID system of embodiment 37, wherein the modification is at the 5′ end of the guide RNA and comprises at least one of 2′-OMe, 2′-O-MOE, inverted abasic, or 2′-F modified nucleotide.

Embodiment 65. The Cas9-IID system of any one of embodiments 1-28 or 37-64, or method of any one of embodiments 30-36, or engineered guide RNA of any one of embodiments 37-64, wherein the guide RNA comprises, in series, a 5′-N region, S1′ region, a S1″ region substantially complementary to the S1′ region, a S2′ region, a S3′ region, a S4′ region, a S4″ region substantially complementary to the S4′ region, a S5′ region, a S5″ region substantially complementary to the S5′ region, a D3″ region substantially complementary to the S3′ region, a S6′ region, a S6″ region substantially complementary to the S6′ region, a S2″ region substantially complementary to the S2′ region, and 3′-tail region, wherein each region is independently from 1 to 25 (e.g., 2, 3, 4, 5, 6, 7, 8, 9, 10, 11, 12, 13, 14, 15, 16, 17, 18, 19, 20, 21, 22, 23, 24 or 25) nucleotides in length, and wherein the regions are connected independently by via 1, 2, 3, 4, 5, 6, 7, 8, or more nucleotides, or connected directly by a modified (e.g., phosphorothioate, imidp, or MMI) or unmodified (e.g., phosphodiester) internucleoside linkage

Embodiment 66. An engineered guide RNA comprising, in series, a 5′-N region, S1′ region, a S1″ region substantially complementary to the S1′ region, a S2′ region, a S3′ region, a S4′ region, a S4″ region substantially complementary to the S4′ region, a S5′ region, a S5″ region substantially complementary to the S5′ region, a D3″ region substantially complementary to the S3′ region, a S6′ region, a S6″ region substantially complementary to the S6′ region, a S2″ region substantially complementary to the S2′ region, and 3′-tail region, wherein the guide RNA is less than 155 (e.g., 154, 153, 152, 151, 150, 149, 148, 147, 146, 145, 144, 143, 142, 141, 140, 139, 138, 137, 136, 135, 134, 133, 132, 131, 130, 129, 128, 127, 126, 125, 124, 123, 122, 121, 120, 119, 118, 117, 116, 115, 114, 113, 112, 111, 110, 109, 108, 107, 106, 105, 104, 103, 102, 101, 100 or less) nucleotides in length, and wherein: (i) 5′-N, S5′, S″, S6′ and S6″ regions are independently absent or independently from 1 to 25 (e.g., 2, 3, 4, 5, 6, 7, 8, 9, 10, 11, 12, 13, 14, 15, 16, 17, 18, 19, 20, 21, 22, 23, 24 or 25) nucleotides in length; (ii) the S1′, S1″, S2′, S2″, S3′, S3″, S4′, S4″, and the 3′-tail regions are independently from 1 to 25 (e.g., 2, 3, 4, 5, 6, 7, 8, 9, 10, 11, 12, 13, 14, 15, 16, 17, 18, 19, 20, 21, 22, 23, 24 or 25) nucleotides in length; (iii) the 5′-N and the S1′ region are connected to each other via 1, 2, 3, 4, 5, 6, 7, 8, or more nucleotides, or directly by a modified (e.g., phosphorothioate, midophosphoramidate (imidp), or methylenemethylimino (MMI, 3′-CH₂—N(CH₃)—O-5′) or unmodified (e.g., phosphodiester) internucleoside linkage; (iv) the S1′ and S1″ regions are connected to each other via 1, 2, 3, 4, 5, 6, 7, 8, or more nucleotides, or directly by a modified (e.g., phosphorothioate, midophosphoramidate (imidp), or methylenemethylimino (MMI, 3′-CH₂—N(CH₃)—O-5′) or unmodified (e.g., phosphodiester) internucleoside linkage; (v) the S2′ and S3′ regions are connected to each other via 1, 2, 3, 4, 5, 6, 7, 8, or more nucleotides, or directly by a modified (e.g., phosphorothioate, midophosphoramidate (imidp), or methylenemethylimino (MMI, 3′-CH₂—N(CH₃)—O-5′) or unmodified (e.g., phosphodiester) internucleoside linkage; (vi) S3′ and S4′ regions are connected to each other via 1, 2, 3, 4, 5, 6, 7, 8, or more nucleotides, or directly by a modified (e.g., phosphorothioate, midophosphoramidate (imidp), or methylenemethylimino (MMI, 3′-CH₂—N(CH₃)—O-5′) or unmodified (e.g., phosphodiester) internucleoside linkage; (vii) the S4′ and S4″ regions are connected to each other via 1, 2, 3, 4, 5, 6, 7, 8, or more nucleotides, or directly by a modified (e.g., phosphorothioate, midophosphoramidate (imidp), or methylenemethylimino (MMI, 3′-CH₂—N(CH₃)—O-5′) or unmodified (e.g., phosphodiester) internucleoside linkage; (viii) the S4″ and S5′ regions are connected to each other via 1, 2, 3, 4, 5, 6, 7, 8, or more nucleotides, or directly by a modified (e.g., phosphorothioate, midophosphoramidate (imidp), or methylenemethylimino (MMI, 3′-CH₂—N(CH₃)—O-5′) or unmodified (e.g., phosphodiester) internucleoside linkage; (ix) the S5′ and S5″ regions are connected to each other via 1, 2, 3, 4, 5, 6, 7, 8, or more nucleotides, or directly by a modified (e.g., phosphorothioate, midophosphoramidate (imidp), or methylenemethylimino (MMI, 3′-CH₂—N(CH₃)—O-5′) or unmodified (e.g., phosphodiester) internucleoside linkage; (x) the S5″ and S3″ regions are connected to each other via 1, 2, 3, 4, 5, 6, 7, 8, or more nucleotides, or directly by a modified (e.g., phosphorothioate, midophosphoramidate (imidp), or methylenemethylimino (MMI, 3′-CH₂—N(CH₃)—O-5′) or unmodified (e.g., phosphodiester) internucleoside linkage; (xi) the S3″ and S6′ regions are connected to each other via 1, 2, 3, 4, 5, 6, 7, 8, or more nucleotides, or directly by a modified (e.g., phosphorothioate, midophosphoramidate (imidp), or methylenemethylimino (MMI, 3′-CH₂—N(CH₃)—O-5′) or unmodified (e.g., phosphodiester) internucleoside linkage; (xii) the S6′ and S6″ regions are connected to each other via 1, 2, 3, 4, 5, 6, 7, 8, or more nucleotides, or directly by a modified (e.g., phosphorothioate, midophosphoramidate (imidp), or methylenemethylimino (MMI, 3′-CH₂—N(CH₃)—O-5′) or unmodified (e.g., phosphodiester) internucleoside linkage; (xii2″ i) the S6″ and the S2″ regions are connected to each other via 1, 2, 3, 4, 5, 6, 7, 8, or more nucleotides, or directly by a modified (e.g., phosphorothioate, midophosphoramidate (imidp), or methylenemethylimino (MMI, 3′-CH₂—N(CH₃)—O-5′) or unmodified (e.g., phosphodiester) internucleoside linkage; (xiv) the S2′ and the 3′-tail regions are connected to each other via 1, 2, 3, 4, 5, 6, 7, 8, or more nucleotides, or directly by a modified (e.g., phosphorothioate, midophosphoramidate (imidp), or methylenemethylimino (MMI, 3′-CH₂—N(CH₃)—O-5′) or unmodified (e.g., phosphodiester) internucleoside linkage, and optionally, the guide RNA does not comprise the nucleotide sequence of SEQ ID NO: 409.

Embodiment 67. The engineered guide RNA of embodiment 66, wherein the guide nucleic acid comprises at least one nucleic acid modification.

Embodiment 68. The engineered guide RNA of embodiment 66 or 67, wherein the guide nucleic acid comprises at least one nucleic acid modification selected from the group consisting of nucleobase modifications (e.g., a non-natural or modified nucleobase), sugar modifications, inter-sugar linkage modifications (e.g., modified internucleotide linkages), conjugates (e.g., ligands), and any combinations thereof. Nucleic acid modifications also include unnatural, or degenerate nucleobases.

Embodiment 69. The engineered guide RNA of any one of embodiments 66-68, wherein the guide RNA comprises a modified nucleotide selected from the group consisting of 2′-O-methyl (2′-OMe) nucleotides, 2′-fluoro (2′-F) nucleotides, bridged nucleic acid (BNA) nucleotides (e.g., 2′-0,4′-C-methylene (locked nucleic acid, LNA) nucleotides, 2′-0,4′-C-ethylene (locked nucleic acid, ENA) nucleotides, 5′-methyl-BNA, cEt BNA, cMOE BNA, oxy amino BNA and vinyl-carbo BNA), anhydrohexitol (1,5-anhydrohexitol nucleic acid, HNA) nucleotides, cyclohexene (Cyclohexene nucleic acid, CeNA) nucleotides, 2′-methoxyethyl (2′-MOE) nucleotides, 2′-O-allyl nucleotides, 2′-C-allyl ribose nucleotides, 2′-O—N-methylacetamido (2′-O-NMA) nucleotides, a 2′-O-dimethylaminoethoxyethyl (2′-O-DMAEOE) nucleotides, 2′-O-aminopropyl (2′-O-AP) nucleotides, 2′-F arabinose (2′-ara-F) nucleotides, threose (Threose nucleic acid, TNA) nucleotides, and acyclic nucleotides (e.g., unlocked nucleic acids (UNA) and 2,3-dihydroxylpropyl (glycol nucleic acid, GNA)); a modified internucleoside linkage; a non-natural or modified nucleobase; or a combination thereof, optionally, the modified nucleotide is a 2′-O-methyl (2′-OMe) nucleotide or a 2′-fluoro nucleotide, and/or a nucleotide comprising a non-natural or modified nucleobase.

Embodiment 70. The engineered guide RNA of any one of embodiments 66-69, wherein the guide RNA comprises at least one (e.g., 1, 2, 3, 4, 5, or more) modified internucleoside linkage (e.g., a modified internucleoside linkage selected independently from the group consisting of phosphorothioates (R, S, or racemic), phosphorodithioates, methylenemethylimino (MMI, 3′-CH₂—N(CH₃)—O-5′), phosphotriesters, alkylphosphonates (e.g., methylphosphonates), phosphoramidate, methylenemethylimino (—CH₂—N(CH₃)—O—CH₂—), thiodiester (—O—C(O)—S—), thionocarbamate (—O—C(O)(NH)—S—), siloxane (—O—Si(H)₂—O— and dialkylsiloxane), N,N′-dimethylhydrazine (—CH₂—N(CH₃)—N(CH₃)—), amide-3 (3′-CH₂—C(═O)—N(H)-5′), amide-4 (3′-CH₂—N(H)—C(═O)-5′)), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methykenecarbonylamino, methylenehydrazo, methylenedimethylhydrazo, methyleneoxymethylimino, ethers (C3′-O—C5′), thioethers (C3′-S—C5′), thioacetamido (C3′-N(H)—C(═O)—CH₂—S—C5′, C3′-O—P(O)—O—SS—C5′), C3′-CH₂—NH—NH—C5′, 3′-NHP(O)(OCH₃)—O-5′, 3′—NHP(O)(OCH₃)—O-5′), 2′->5′ internucleoside linkages, 2′->3′ internucleoside linkages, 3′->3′ internucleoside linkages, and 5′->5′ internucleoside linkages, optionally the modified internucleoside linkage is phosphorothioate, imidp or MMI, more optionally the modified internucleoside linkage is phosphorothioate (PS).

Embodiment 71. The engineered guide RNA of any one of embodiments 66-70, wherein the guide RNA comprises at least one duplex stabilizing modification.

Embodiment 72. The engineered guide RNA of any one of embodiments 66-71, wherein the guide RNA comprises at least one duplex stabilizing modification selected from the group consisting of 2′-F nucleotides, 2′-OMe nucleotides, 2′-methoxyethyl nucleotides, 2,6-diaminopurine nucleotides, 5-methyl cytidine, N4-ethyl cytidine, 5-propynyl cytidine, 5-propynyl uridine, 5-hydroxybutynl-2′-deoxyuridine, 8-aza-7-deazaguanosine, a locked nucleic acid (LNA), and/or covalent cross-linking of two strands in the duplex.

Embodiment 73. The engineered guide RNA of any one of embodiments 66-72, wherein the 3′-tail region comprises any one of: (i) a modification of any one or more of the last 7, 6, 5, 4, 3, 2, or 1 nucleotides; (ii) one modified nucleotide; (ii) two modified nucleotides; (iii) three modified nucleotides; (iv) four modified nucleotides; (v) five modified nucleotides; (vi) six modified nucleotides; or (vii) seven modified nucleotides.

Embodiment 74. The engineered guide RNA of any one of embodiments 66-72, wherein, the 3′-tail region comprises: (i) a modified internucleoside linkage (e.g., PS, imidp or MMI linkage) between nucleotides; (ii) a 2′-OMe nucleotide; (iii) a 2′-O-MOE nucleotide; (iv) a 2′-F nucleotide; (v) a LNA nucleotide; (vi) a 3′->3′ linkage between nucleotides; (vii) an inverted abasic nucleotide; or (viii) a combination of one or more of (i)-(vii).

Embodiment 75. The engineered guide RNA of any one of embodiments 66-72, wherein the 3′-tail region comprises: (i) a modified internucleoside linkage (e.g., PS, imidp or MMI linkage) between the last and second to last nucleotides; (ii) a modified internucleoside linkage (e.g., PS, imidp or MMI linkage) between the last and second to last nucleotide and a PS linkage between the second and third to last nucleotides; (iii) modified internucleoside linkages (e.g., PS and/or MMI linkage) between any one or more of the last four nucleotides; (iv) modified internucleoside linkages (e.g., PS and/or MMI linkage) between any one or more of the last five nucleotides; and/or or (v) modified internucleoside linkages (e.g., PS and/or MMI linkage) between any one or more of the last 2, 3, 4, 5, 6, 7, 8, 9, or 10 nucleotides.

Embodiment 76. The engineered guide RNA of any one of embodiments 66-72, wherein the 3′-tail region comprises: (i) a modification of one or more of the last 1-7 nucleotides, wherein the modification is a modified internucleoside linkages (e.g., PS and/or MMI linkage), inverted abasic nucleotide, a 3′->3′ internucleoside linkage, 2′-OMe, 2′-O-MOE, 2′-F, LNA, or a combination thereof; (ii) a modification to the last nucleotide with 2′-OMe, 2′-O-MOE, 2′-F, LNA, or combinations thereof, and an optional one or two modified internucleoside linkages (e.g., PS and/or MMI linkage) to the next nucleotide; (iii) a modification to the last and/or second to last nucleotide with 2′-OMe, 2′-O-MOE, 2′-F, LNA, or combinations thereof, and optionally one or more modified internucleoside linkages (e.g., PS and/or MMI linkage); iv. a modification to the last, second to last, and/or third to last nucleotides with 2′-OMe, 2′-O-moe, 2′-F, or combinations thereof, and optionally one or more modified internucleoside linkages (e.g., PS and/or MMI linkage); (v) a modification to the last, second to last, third to last, and/or fourth to last nucleotides with 2′-OMe, 2′-O-MOE, 2′-F, LNA or combinations thereof, and optionally one or more modified internucleoside linkages (e.g., PS and/or MMI linkage); and/or (vi) a modification to the last, second to last, third to last, fourth to last, and/or fifth to last nucleotides with 2′-OMe, 2′-O-MOE, 2′-F, LNA, or combinations thereof, and optionally one or more modified internucleoside linkages (e.g., PS and/or MMI linkage).

Embodiment 77. The engineered guide RNA of any one of embodiments 66-72, wherein the 3′-tail region comprises: (i) a 2′-OMe modified nucleotide at the last position, three consecutive 2′-O-MOE modified nucleotides immediately 5′ to the 2′-OMe modified nucleotide, and three consecutive PS linkages between the last three nucleotides; (ii) five consecutive 2′-OMe modified nucleotides from the 3′ end of the 3′ terminus, and three PS linkages between the last three nucleotides; (iii) an inverted abasic modified nucleotide at the last position; (iv) an inverted abasic modified nucleotide at the last position, and three consecutive 2′-OMe modified nucleotides at the last three positions; (v) 15 consecutive 2′-OMe modified nucleotides from the 3′ end, five consecutive 2′-F modified nucleotides immediately 5′ to the 2′-OMe modified nucleotides, and three PS linkages between the last three nucleotides; (vi) alternating 2′-OMe modified nucleotides and 2′-F modified nucleotides at the last 20 nucleotides, and three PS linkages between the last three nucleotides; (vii) two or three consecutive 2′-OMe modified nucleotides, and three PS linkages between the last three nucleotides; (viii) one PS linkage between the last and next to last nucleotides; and/or (ix) 15 or 20 consecutive 2′-OMe modified nucleotides, and three PS linkages between the last three nucleotides.

Embodiment 78. The engineered guide RNA of any one of embodiments 66-77, wherein the guide RNA comprises, at its 5′-end, any one of: (i) one modified nucleotide; (ii) two modified nucleotides; (iii) three modified nucleotides; (iv) four modified nucleotides; (v) five modified nucleotides; (vi) six modified nucleotides; or (vii) seven modified nucleotides, optionally wherein the guide RNA comprises, at its 5′-end, between 1 and 7, between 1 and 5, between 1 and 4, between 1 and 3, or between 1 and 2 modified nucleotides.

Embodiment 79. The engineered guide RNA of any one of embodiments 66-77, wherein the, the guide RNA comprises, at its 5′-end, one or more of: (i) a modified internucleoside linkage (e.g., a phosphorothioate and/or MMI linkage) between nucleotides; (ii) a 2′-OMe nucleotide; (iii) a 2′-O-MOE nucleotide; (iv) a 2′-F nucleotide; (v) a LNA; (vi) a 3′->3′ linkage; (vii) an inverted abasic modified nucleotide; (viii) a deoxyribonucleotide; (ix) an inosine; and (x) combinations of one or more of (i)-(ix).

Embodiment 80. The engineered guide RNA of any one of embodiments 66-77, wherein the guide RNA comprises, at its 5′-end, about 1-2, 1-3, 1-4, 1-5, 1-6, or 1-7 modified internucleoside (e.g., a phosphorothioate and/or MMI) linkages between nucleotides, optionally wherein the guide RNA comprises, at its 5′-end, 1, 2, 3, 4, 5, 6, and/or 7 modified internucleoside (e.g., a phosphorothioate and/or MMI) linkages between nucleotides. In some embodiments, the guide RNA comprises, at its 5-end, any one of: (i) one modified internucleoside (e.g., a phosphorothioate and/or MMI) linkage, and the linkage is between nucleotides 1 and 2; (ii) two modified internucleoside (e.g., a phosphorothioate and/or MMI) linkages, and the linkages are between nucleotides 1 and 2, and 2 and 3; (iii) modified internucleoside (e.g., a phosphorothioate and/or MMI) linkages between any one or more of nucleotides 1 and 2, 2 and 3, and 3 and 4; (iv) modified internucleoside (e.g., a phosphorothioate and/or MMI) linkages between any one or more of nucleotides 1 and 2, 2 and 3, 3 and 4, and 4 and 5; (v) modified internucleoside (e.g., a phosphorothioate and/or MMI) linkages between any one or more of nucleotides 1 and 2, 2 and 3, 3 and 4, 4 and 5, and 5 and 6; (vi) modified internucleoside (e.g., a phosphorothioate and/or MMI) linkages between any one or more of nucleotides 1 and 2, 2 and 3, 3 and 4, 4 and 5, 5 and 6, and 6 and 7; or (vii) modified internucleoside (e.g., a phosphorothioate and/or MMI) linkages between any one or more of nucleotides 1 and 2, 2 and 3, 3 and 4, 4 and 5, 5 and 6, 6 and 7, and 7 and 8.

Embodiment 81. The engineered guide RNA of any one of embodiments 66-77, wherein the, the guide RNA comprises, at its 5-end, at least one 2′-OMe, 2′-O-MOE, inverted abasic, or 2′-F modified nucleotide.

Embodiment 82. The engineered guide RNA of any one of embodiments 66-81, wherein the S1′ and S1″ regions together form a double stranded structure (duplex region), optionally the duplex comprises an internal loop comprising 2 to 10 (e.g., 2, 3, 4, 5, 6, 7, 8 or 9) nucleotides, and/or a bulge loop comprising 1 to 10 (e.g., 2, 3, 4, 5, 6, 7, 8 or 9), more optionally, the duplex comprises an internal loop comprising 2 to 10 (e.g., 2, 3, 4, 5, 6, 7, 8 or 9) nucleotides.

Embodiment 83. The engineered guide RNA of any one of embodiments 66-82, wherein the S2′ and S2″ regions together form a double stranded structure), optionally the duplex comprises an internal loop comprising 2 to 10 (e.g., 2, 3, 4, 5, 6, 7, 8 or 9) nucleotides, and/or a bulge loop comprising 1 to 10 (e.g., 2, 3, 4, 5, 6, 7, 8 or 9) nucleotides, preferably, the duplex does not comprise an bulge or internal loop.

Embodiment 84. The engineered guide RNA of any one of embodiments 66-83, wherein the S3′ and S3″ regions together form a double stranded structure), optionally the duplex comprises an internal loop comprising 2 to 10 (e.g., 2, 3, 4, 5, 6, 7, 8 or 9) nucleotides, and/or a bulge loop comprising 1 to 10 (e.g., 2, 3, 4, 5, 6, 7, 8 or 9) nucleotides, more optionally, the duplex does not comprise a bulge or internal loop, or the duplex region comprises a 1 nucleotide bulge, preferably, the duplex does not comprise a bulge or internal loop.

Embodiment 85. The engineered guide RNA of any one of embodiments 66-84, wherein the S4′ and S4″ regions together form a double stranded structure), optionally the duplex comprises an internal loop comprising 2 to 10 (e.g., 2, 3, 4, 5, 6, 7, 8 or 9) nucleotides, and/or a bulge loop comprising 1 to 10 (e.g., 2, 3, 4, 5, 6, 7, 8 or 9) nucleotides, optionally, the duplex comprises an internal loop comprising 2 to 10 (e.g., 2, 3, 4, 5, 6, 7, 8 or 9) nucleotides.

Embodiment 86. The engineered guide RNA of any one of embodiments 66-83, wherein the S5′ and S5″ regions together form a double stranded structure, optionally the duplex comprises an internal loop comprising 2 to 10 (e.g., 2, 3, 4, 5, 6, 7, 8 or 9) nucleotides, and/or a bulge loop comprising 1 to 10 (e.g., 2, 3, 4, 5, 6, 7, 8 or 9) nucleotides, preferably, the duplex comprises an internal loop comprising 2 to 10 (e.g., 2, 3, 4, 5, 6, 7, 8 or 9) nucleotides.

Embodiment 87. The engineered guide RNA of any one of embodiments 66-86, wherein the S6′ and S6″ region regions together form a double stranded structure, optionally the duplex comprises an internal loop comprising 2 to 10 (e.g., 2, 3, 4, 5, 6, 7, 8 or 9) nucleotides, and/or a bulge loop comprising 1 to 10 (e.g., 2, 3, 4, 5, 6, 7, 8 or 9) nucleotides, preferably, the duplex does not comprises an internal loop.

Embodiment 88. The engineered guide RNA of any one of embodiments 66-87, wherein the S5′ and S5″ regions are absent.

Embodiment 89. The engineered guide RNA of any one of embodiments 66-88, wherein the S 6′ and S6″ regions are absent.

Embodiment 90. The engineered guide RNA of any one of embodiments 66-89, wherein the S5′, S5″, S6′ and S6″ regions are absent.

Embodiment 91. The engineered guide RNA of any one of embodiments 66-90, wherein the guide RNA is at least 1 (e.g., 2, 3, 4, 5, 6, 7, 8, 9, 10, 11, 12, 13, 14, 15, 16, 17, 18, 19, 20 or more) nucleotides shorter than SEQ ID NO: 409, optionally, the guide RNA is at least 5, 10, 15, 20, 25 or more nucleotides shorter than SEQ ID NO: 409.

Embodiment 92. The engineered guide RNA of any one of embodiments 66-91, wherein the guide RNA comprises a nucleotide sequence having at least 60%, 65%, 70%, 75%, 80%, 85%, 90%, 95% identity to a nucleic acid selected from the group consisting of SEQ ID NOs: 315-408, 410-506, 509-756 or 759-772.

Embodiment 93. The Cas9-IID system of any one of embodiments 1-28 or 37-64, wherein the guide RNA is an engineered guide RNA of any one of embodiments 66-92.

For all embodiments of the systems and methods described herein, it is known in the field that complete complementarity is not required for hybridization or binding as described herein, provided that there is sufficient complementarity to be functional. Modulations of cleavage efficiency can be exploited by introducing mismatches, e.g., one or more mismatches, such as 1 or 2 mismatches between a spacer sequence and a target sequence, including the position of the mismatch along the spacer/target. The more central (i.e., not at the 3′ or 5′ ends) a mismatch, e.g., a double mismatch, is located; the more cleavage efficiency is affected. Accordingly, by choosing mismatch positions along the spacer sequence, cleavage efficiency can be modulated. For example, if less than 100% cleavage of targets is desired (e.g., in a cell population), 1 or 2 mismatches between spacer and target sequence can be introduced in the spacer sequences.

### Optimization of CRISPR Systems for Use in Select Organisms

### Codon-Optimization

The invention contemplates all possible variations of nucleic acids, such as cDNA, that could be made by selecting combinations based on possible codon choices. These combinations are made in accordance with the standard triplet genetic code as applied to the polynucleotide encoding naturally occurring variant, and all such variations are to be considered as being specifically disclosed. Nucleotide sequences encoding type V-G CRISPR-Cas-associated effector protein variants that have been codon-optimized for expression in bacteria (e.g., E. coli) and in human cells are disclosed herein. For example, the codon-optimized sequences for human cells can be generated by substituting codons in the nucleotide sequence that occur at lower frequency in human cells for codons that occur at higher frequency in human cells. The frequency of occurrence for codons can be computationally determined by methods known in the art. An exemplary calculation of these codon frequencies for various host cells (e.g., E. coli, yeast, insect, C. elegans, D. melanogaster, human, mouse, rat, pig, P. pastoris, A. thalian, maize, and tobacco) have been published or made available by sources such as the GenScript® Codon.

### Definitions

For convenience, the meaning of some terms and phrases used in the specification, examples, and appended claims, are provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

The term “Ins” as used herein refers to an insertion after the indicated position. In some embodiments, the inserted amino acid sequence is NKKKSRR (SEQ ID NO: 507). For example, the mutation L363Ins indicates NKKKSRR (SEQ ID NO: 507) is inserted after L363 of SEQ ID NO: 1 or SEQ ID NO: 12. As another example, the mutation L356Ins indicates NKKKSRR (SEQ ID NO: 507) is inserted after L356 of SEQ ID NO: 27. In some embodiments, the inserted amino acid is R. For example, the mutation K694Ins indicates R is inserted after K694 of SEQ ID NO: 1. In some embodiments, the inserted amino acid sequence is ANKKTSP (SEQ ID NO: 508). For example, the mutation K692Ins indicates ANKKTSP (SEQ ID NO: 508) is inserted after K692 of SEQ ID NO: 1.

Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviation, per the practice in the art. Alternatively, “about” can mean a range of up to 20%, up to 15%, up to 10%, up to 5%, or up to 1% of a given value.

As used herein, a “cell” generally refers to a biological cell. A cell can be a single cell as well as to a population of (i.e., more than one) cells. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g. Botryococcus braunii, Chlamydomonas reinhardtii, Nannochloropsis gaditana, Chlorella pyrenoidosa, Sargassum patens C. Agardh, and the like), seaweeds (e.g., kelp), a fungal cell (e.g., a yeast cell, a cell from a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etc. Sometimes a cell is not originating from a natural organism (e.g., a cell can be a synthetically made, sometimes termed an artificial cell). In some embodiments, cell is a human cell.

Cells suitable for use in the present invention include, but are not limited to, cells that are capable of differentiating completely or partially into a mature cell of the inner ear, e.g., a hair cell (e.g., an inner and/or outer hair cell), when contacted, e.g., in vitro, with one or more of the compounds described herein. Exemplary cells that are capable of differentiating into a hair cell include, but are not limited to stem cells (e.g., inner ear stem cells, adult stem cells, bone marrow derived stem cells, embryonic stem cells, mesenchymal stem cells, skin stem cells, iPS cells, and fat derived stem cells), progenitor cells (e.g., inner ear progenitor cells), support cells (e.g., Deiters' cells, pillar cells, inner phalangeal cells, tectal cells and Hensen's cells), and/or germ cells.

The cell may be a prokaryotic cell or a eukaryotic cell. The cell may be a mammalian cell. The mammalian cell many be a non-human primate, bovine, porcine, rodent or mouse cell. The cell may be a non-mammalian eukaryotic cell such as poultry, fish or shrimp. The cell may be a therapeutic T cell or antibody-producing B-cell. The cell may also be a plant cell. The plant cell may be of a crop plant such as cassava, corn, sorghum, wheat, or rice. The plant cell may also be of an algae, tree or vegetable. The modification introduced to the cell by the present invention may be such that the cell and progeny of the cell are altered for improved production of biologic products such as an antibody, starch, alcohol or other desired cellular output. The modification introduced to the cell by the present invention may be such that the cell and progeny of the cell include an alteration that changes the biologic product produced.

The terms “polynucleotide,” “oligonucleotide,” and “nucleic acid” are used interchangeably to generally refer to a polymeric form of nucleotides of any length, either deoxyribonucleotides or ribonucleotides, or analogs thereof, either in single-, double-, or multi-stranded form. A polynucleotide may be exogenous or endogenous to a cell. A polynucleotide may exist in a cell-free environment. A polynucleotide may be a gene or fragment thereof. A polynucleotide may be DNA. A polynucleotide may be RNA. A polynucleotide may have any three-dimensional structure and may perform any function. A polynucleotide may comprise one or more nucleic acid modifications. If present, modifications to the nucleotide structure may be imparted before or after assembly of the polymer. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides may be interrupted by non-nucleotide components.

Nucleic acid sequences that are at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% identical to the nucleotide sequences described herein are also specifically contemplated and provided for herein. In some embodiments, the nucleic acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are the same as a nucleotide sequence described herein. In some embodiments, the nucleic acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from a nucleotide sequence described herein.

The terms “transfection” or “transfected” generally refer to introduction of a nucleic acid into a cell by non-viral or viral-based methods. The nucleic acid molecules may be gene sequences encoding complete proteins or functional portions thereof. See, e.g., Sambrook et ah, 1989, Molecular Cloning: A Laboratory Manual, 18.1-18.88. The terms “peptide,” “polypeptide,” and “protein” are used interchangeably herein to generally refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer may be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids may include natural amino acids and non-natural amino acids, which have been chemically modified to include a group or a chemical moiety not naturally present on the amino acid. Amino acid analogues may refer to amino acid derivatives. The term “amino acid” includes both D-amino acids and L-amino acids.

Amino acid sequences that are at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% identical to an amino acid sequence described herein are also specifically contemplated and provided for herein. In some embodiments, the amino acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a amino acid sequence described herein. In some embodiments, the amino acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from the sequences described herein.

The terms “wild-type” or “wt” or “native” as used herein is meant an amino acid sequence or a nucleotide sequence that is found in nature, including allelic variations. A wild-type protein, polypeptide, antibody, immunoglobulin, IgG, polynucleotide, DNA, RNA, and the like has an amino acid sequence or a nucleotide sequence that has not been intentionally modified

As used herein, the term “non-native” can generally refer to a nucleic acid or polypeptide sequence that is not found in a native nucleic acid or protein. Non-native may refer to affinity tags. Non-native may refer to fusions. Non-native may refer to a naturally occurring nucleic acid or polypeptide sequence that comprises mutations, insertions and/or deletions. A non-native sequence may exhibit and/or encode for an activity (e.g., enzymatic activity, methyltransferase activity, acetyltransferase activity, kinase activity, ubiquitinating activity, etc.) that may also be exhibited by the nucleic acid and/or polypeptide sequence to which the non-native sequence is fused. A non-native nucleic acid or polypeptide sequence may be linked to a naturally occurring nucleic acid or polypeptide sequence (or a variant thereof) by genetic engineering to generate a chimeric nucleic acid and/or polypeptide sequence encoding a chimeric nucleic acid and/or polypeptide.

The term “promoter”, as used herein, generally refers to the regulatory DNA region which controls transcription or expression of a gene and which may be located adjacent to or overlapping a nucleotide or region of nucleotides at which RNA transcription is initiated. A promoter may contain specific DNA sequences which bind protein factors, often referred to as transcription factors, which facilitate binding of RNA polymerase to the DNA leading to gene transcription. A ‘basal promoter’, also referred to as a ‘core promoter’, may generally refer to a promoter that contains all the basic necessary elements to promote transcriptional expression of an operably linked polynucleotide. Eukaryotic basal promoters typically, though not necessarily, contain a TATA-box and/or a CAAT box.

The term “expression”, as used herein, generally refers to the process by which a nucleic acid sequence or a polynucleotide is transcribed from a DNA template (such as into mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, “operably linked”, “operable linkage”, “operatively linked”, or grammatical equivalents thereof generally refer to juxtaposition of genetic elements, e.g., a promoter, an enhancer, a polyadenylation sequence, etc., wherein the elements are in a relationship permitting them to operate in the expected manner. For instance, a regulatory element, which may comprise promoter and/or enhancer sequences, is operatively linked to a coding region if the regulatory element helps initiate transcription of the coding sequence. There may be intervening residues between the regulatory element and coding region so long as this functional relationship is maintained.

A “functional fragment” of a DNA or protein sequence generally refers to a fragment that retains a biological activity (either functional or structural) that is substantially similar to a biological activity of the full-length DNA or protein sequence. A biological activity of a DNA sequence may be its ability to influence expression in a manner known to be attributed to the full-length sequence.

As used herein, an “engineered” object generally indicates that the object has been modified by human intervention. According to non-limiting examples: a nucleic acid may be modified by changing its sequence to a sequence that does not occur in nature; a nucleic acid may be modified by ligating it to a nucleic acid that it does not associate with in nature such that the ligated product possesses a function not present in the original nucleic acid; an engineered nucleic acid may synthesized in vitro with a sequence that does not exist in nature; a protein may be modified by changing its amino acid sequence to a sequence that does not exist in nature; an engineered protein may acquire a new function or property. An “engineered” system comprises at least one engineered component.

As used herein, “synthetic” and “artificial” are used interchangeably to refer to a protein or a domain thereof that has low sequence identity (e.g., less than 50% sequence identity, less than 25% sequence identity, less than 10% sequence identity, less than 5% sequence identity, less than 1% sequence identity) to a naturally occurring human protein.

The term “sequence identity” or “percent identity” in the context of two or more nucleic acids or polypeptide sequences, generally refers to two (e.g., in a pairwise alignment) or more (e.g., in a multiple sequence alignment) sequences that are the same or have a specified percentage of amino acid residues or nucleotides that are the same, when compared and aligned for maximum correspondence over a local or global comparison window, as measured using a sequence comparison algorithm. Suitable sequence comparison algorithms for polypeptide sequences include, e.g., BLASTP using parameters of a wordlength (W) of 3, an expectation I of 10, and the BLOSUM62 scoring matrix setting gap costs at existence of 11, extension of 1, and using a conditional compositional score matrix adjustment for polypeptide sequences longer than 30 residues; BLASTP using parameters of a wordlength (W) of 2, an expectation (E) of 1000000, and the PAM30 scoring matrix setting gap costs at 9 to open gaps and 1 to extend gaps for sequences of less than 30 residues (these are the default parameters for BLASTP in the BLAST suite available at https://blast.ncbi.nlm.nih.gov); CLUSTALW with parameters of the Smith-Waterman homology search algorithm with parameters of a match of 2, a mismatch of −1, and a gap of −1; MUSCLE with default parameters; MAFFT with parameters retree of 2 and maxiterations of 1000; Novafold with default parameters; HMMER hmmalign with default parameters.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. a control level) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

As used herein, the term “Recombinase” refers to an enzyme that catalyzes recombination between two or more recombination sites (e.g., an acceptor and donor site). Recombinases useful in the present invention catalyze recombination at specific recombination sites which are specific polynucleotide sequences that are recognized by a particular recombinase. “Uni-directional recombinases” or “integrases” refer to recombinase enzymes whose recognition sites are destroyed after the recombination has taken place. The term “integrase” refers to a type of recombinase. In other words, the sequence recognized by the recombinase is changed into one that is not recognized by the recombinase upon recombination. As a result, once a sequence is subjected to recombination by the unidirectional recombinase, the continued presence of the recombinase cannot reverse the previous recombination event.

“Recombination sites” are specific polynucleotide sequences that are recognized by the recombinase enzymes described herein. Typically, two different sites are involved (in regards to recombination termed “complementary sites”), one present in the target nucleic acid (e.g., a chromosome or episome of a eukaryote) and another on the nucleic acid that is to be integrated at the target recombination site. The terms “attB” and “attP,” which refer to attachment (or recombination) sites originally from a bacterial target (attachment site of bacteria) and a phage donor (attachment site of phage), respectively, are used herein although recombination sites for particular enzymes may have different names. The two attachment sites can share as little sequence identity as a few base pairs. The recombination sites typically include left and right arms separated by a core or spacer region. Thus, an attB recombination site consists of BOB′, where B and B′ are the left and right arms, respectively, and O is the core region. Similarly, attP is POP′, where P and P are the arms and O is again the core region. Upon recombination between the attB and attP sites, and concomitant integration of a nucleic acid at the target, the recombination sites that flank the integrated DNA are referred to as “attL” and “aatR.” The attL and attR sites, using the terminology above, thus consist of BOP′ and POB′, respectively. In some representations herein, the “O” is omitted and attB and attP, for example, are designated as BB′ and PP′, respectively.

The terms “increased”, “increase”, “enhance”, or “activate” are all used herein to mean an increase by a statistically significant amount. In some embodiments, the terms “increased”, “increase”, “enhance”, or “activate” can mean an increase of at least 10% as compared to a reference level (e.g., a control level), for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of a disease or disorder. A subject can be male or female.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

### EXAMPLES

The technology described herein is further illustrated by the following examples which in no way should be construed as being further limiting.

### Example 1—Genome Cleavage Activity of Cas9 ID Proteins in Mammalian Cells

To demonstrate targeting and cleavage activity in mammalian cells, plasmids expressing the Cas9 IID protein sequences (SEQ ID NOs: 1-12) and the engineered single guide RNA sequences (SEQ ID NO: 13-24) were transfected into mammalian cells. Two plasmids were cotransfected; the first plasmid was a protein expression plasmid that uses a mammalian expression backbone, pcDNA3.1, with the CMV promoter. This plasmid drives expression of a human codon optimized Cas9 IID protein with a N and C-term SV40 NLS tags.

The second plasmid used a human U6 promoter to drive expression of the sgRNA. The two plasmids were cotransfected into HEK293FT cells and genomic DNA is harvested 72 hrs after cotransfection. The target sequence in the genomic DNA was amplified and sequenced using NGS. Targeting WT Cas9 IID RNP complexes led to dsDNA breaks which were typically repaired using a cellular mechanism known as Non-Homologous End Joining (NHEJ), which produced insertions or deletions (indels) at the cut site. The frequency of indels at the targeted genomic locus were measured to demonstrate the targeting efficiency of the enzyme in mammalian cells. At least 8 different target sites were chosen to test each protein's activity.

### Cas9 IID Protein Sequences:

>25-_5028637.4360_-_Cas9_CDS_translation
(SEQ ID NO: 1)
MITLGIDYGASNIGIALVLTTEAGENIPLFAGTLRVDARHLKEKVETRAGIRRLRRTRKTKKRR
LRNLQHALESLGLSPDQTSKIIRFSKRRGYKSLFDKDTPDETKDDSELTYRFTREEFFKSLEKE
LSEWISDEVKRAKALSICEKILNRHGNRDHEIRKLRIDNRGVSRCAWEGCRAVTPRLENALKE
ALSQQLYTVFQTLVRENTAIRNEIDEAVANLTELAKRLRNASGDDANSEKKILRKKARSVLR
HLRDRFFALDEPGLEKDKAWKYIESSLMNTLENRGGRNRYCRFHSNEYINTILQGKPVPFKK
TIADSDIISRREQIAYAKIWRYIEARLLPLAPEGIDRIVVERTAFDLLAGSWKSIADATDAFKED
MYQQGPMYGFSSRGEMLREEFGGLCAYCGLPSSVLMDRDHILPRADFFFDSYLNIVPACPKC
NSALKGRRPASEIALTIHPDAYNAYDHYLRSKFKKRPMHFFHTIKKGILNLMKDPDRLWEAE
RYLSLITKQFSQIVQTQRSPRPFARYLSSKIGTLQGKTPEIRFRNGRHTALYRNVAYPEFDKYR
EKAEGNAINHALDAMLLASELPDLYPVESLNIPLSSLKAWSASVKRKAPKPSADGIPSIQNLN
DFVDGFEVVHGDGYVDVALRTMVWNQRDTMTHKQDPYGWSAKDKRPTKRTSALDLYTEL
KKEKAGKVKNKVELIHHPSLRKAVSDAVTPENPGGTAAEALKLWLCNSVRNTLKTSHFSNH
PGDRARKEALEKFASNENETIPAVIGVKMFDLGVQGKIDLERLDRQTGNVGHRYMTQPPNR
GVIVAYPKASDGHPDMARPCCLYLRQNGAVIPEGLAIFKPLPDKLSKGRIFGSHNNEVSTLFR
DVEDYLKDCGFFGYILLTPGCVAHYADGHSWFVRNFDQSKDFKKFRLRNIVAIRKTPFSQKLI
PQKVLT
>37-_6600918.19360_-_Cas9_CDS + ATG_translation
(SEQ ID NO: 2)
MVATTFTVSIDFGSKYIGIALIMHSPAAPNRVLYAAVIVVEAKPLNASINPRTVARRIRRTGKT
HRRRLRRLGLALGGIRGAEDVLRFCRRRGYAYDPGEEGDETELAHAVSRDEFFEALAAEVD
RVVPERDRGYVLRRCGKHLNAERRGEAELRPARFENRHPSKCQWEGCTRNVPRKGNALRE
QLAQTLFVWLRPIFDEIGNKGPLRDAVERQIDRMVGLARGYANRPDADEKKALSKQKKRAF
ADMLDCVADHGDERTVKRFVDNWRKTYSRQLTAILTKKQGGRLRYCRRHSREYVDVFLAG
KQPPHRTEVHITDLFGRSQQILFERIWRLVHARILPLANGQIDRVVVERVAFDVLAGPFKQRV
KLSEDRAAEMYWHGPMLGFDSRPEMLKEEFDGRCAYCGRKRKLSEVEHILPRSRFPFDSYFN
ILPSCAECNRGKGARSLLEAGKTVDEKAFEAYSDYIGKKRPPHLFHTIKKGMLKLMTDGGSM
ATAERQLALLADNLVSITNTQKSPRPLARFLATEIEKLTGHDCKPQWLSGRHTALYREIILPEY
DKKEEKENGGLVNHAVDAIVAGCKLPSAAALENPRWYTNQNDGPKQSDMLAWRKKVLSV
APELAGRLPRVEPIERLEFFENDLGDGYVKIDLSAFNWNQQRKSGHKLDPFGTTADGKPLKR
KAADDVLKTLLIESKRDGQIAAIAHRGLRQLLERHRKQAARQFVCWLQKTTRKGLADGKRG
THPSDAARYAALEAFVNTPADKFLSNNSATGNETEDKSDEERETIPATIGIRCINAGVKGRLG
VTRCNAHHEKGQLLVSHPQYREFYIGYAAGDDGTPDRSRPIVFSVNQAHA VMRRIGSRVEPV
TDDPTSVLQGIPLGGWGDRKGFLAQWRAELHAVFEQHGIVRWFRVGQGCYIEKTDGTAFQL
CNFDDQKEWMKNGPFKNIQRVYRSPLRGAVERANP
>42-_8178755.467_-_Cas9_CDS_translation
(SEQ ID NO: 3)
MITLGIDYGSSNIGIALVRNTGEGNEPLFAGTIKIDARRLRDKVETRAGIRRLRRTRKTKRHRL
RNLRNRFLSLGIKEEDVNLIVGFCKRRGYKSLFDDGETVESEKGDNQLTYRFTREEFFRNLTD
ELKTILPDSSQFQDALNACEKILNRRGDPFQEIRLIRIDNRGASRCAWEGCDKVTPRRDNALG
DPIAQSLLNAVQENLKGEPARMGTIETDISELDTLGRRIRAASGEGAKEEKKALRKNARKVLR
EIKADFFKPDVDEEERDRAWKYIEAGILNIMENSGGRNRYCREHSKAYIQTVLSGKAAPFKRT
ISDSDIISRREQIVYQKIWRYLEARVFPLAPEGIDRIVVERTAFDLLAGSRKNIQNASDQFVEEM
YQHGPMHGFSSTAEMLKEEFAGMCAYCGCESAALIDRDHILPRADFFFDSYLNIVPACPKCN
SDLKGKRPISTSLLRIDDKAYEAYSHYLQKISKSRPMHLFHTIKKGVLNLMRDPERSWEVERY
LSLIAKQFSEIVQTQRSPRPFARYLTSKLIKRQEKMPEILFRSGRHINLYRTISYPAFSKVEEKEE
GNTANHAIDAMLLASDLPSPSPLEALSIPVSLVKRWSLSVQESAPKTGRNGIPEIPCHGSYVDG
FEKVDGNGYVEIELDKMSWNQKDRMTHKQDPYGWSQKAQMPTKRTAAVDLYNSLKKESN
VQKVKNIIERIHHPALKRVMAASANSENPGGSVAETMKKWLRQSVQNSINNSSFSNHPGDQA
RKGDLEKFTSEETAIPAVIGIKMFDTGVRGKIDLSRIDKQTGKVGHRYMTQPANRGVILAYPK
KPSGEPDTDRPYLAFIKQDSSLKPEGAMFKPLPEGILNGKILGTGYSPGEWMGQVENYLSECG
FHSYVSLTPGCVVCYKNGKKWFVRNFDQSADFKKARLKDVAGIQRTPFITRISPLKMLT
>43-_8858235.1405_-_Cas9_CDS_translation
(SEQ ID NO: 4)
MTHTLSIDFGSTYIGLSLLAHNSADPNKVLYAATVVADPAWLTKTVEPRGQIRRMRRTRKTH
RRRLHRLAQAMAGIQGATQVLSFCRRRGYSHDSDDDDQDTAAFRVPRAAFFRALLAEVERC
VPAADRERVRAACSRHLNEQRTPGAELRPARFDNRGSSRCQWAGCNHNVPRAENCAQEKLS
QALFAWLKPVFDGSADPIRLRRSIDHWIGELAGLSRAYRRAAKLEDESKRDENESGIKRRVK
RVYKNLKERVCREATPEVAESFSESWNEFYQKNLSEIVYGKRGGRAKYCREHSQQFVEMFL
AGQQIPNRQDLSDRDLASLKQQIIFRRLWRLLENRVLPLAGGTIDRLIVERVAIDTLGGLFKDR
QQISDKKAGPLYWYGPQYRFNSRLEMLKEEFDGRCAYCGQAANLVDVEHILPHSAFPFDSYL
NVVPACDDCNRRKGARTPLQAGMTINENAFDAYVNYVRRQKPPHRLHEIKKGMLKLLLRPG
QDGQPERMLGMIANNLVQVTNTQRGPRPLARYLASKLETATGNRPAIAFVSGRHTALYRRV
VLPEYDKPAEKESGDVRNHAVDAILLGCQFPSAAALENQTWYRTTADVVTWCNKVRAVSPP
LDDGVPEVARHDLVPFFETDLGGGYIGIDLSAFNWNHGRRGTHDLDPFAITRSGKPAKRVPA
AKVLAELLLDAGRRDKQIAGIANRGLRNLLEAAPQNAPMAFVTWLQQTTRDGLADGTMGN
HPADQARRQQLEKFVAATVENVIADDEPIPAVVGVRCISNISAGLVEVPRCDRSGRVHQHYK
ANPPVREFYVGYRSKDGAIDGSRPIVFSVNQVYLVRREEGGKKVGLDLPPGSPLLGRPLGAQ
GRLRDFLVAWRSAFDELCRAEGISRRFRVTQGCVIEKVDGSLFQLRNFDKSEPWRKAASFRNI
HRIHRSPLTATDP
>49-_8932059.46230_-_Cas9_CDS_translation
(SEQ ID NO: 5)
MITLAIDYGASNIGIALVRNTEEGNEPLFAGTIIIDAKGVKDLAEPRAAIRRLRRTRKTKIRRLR
QLSLALKRLGLGEEVSRIVRFARRRGYKSLFDDPDEAKQTKDDVSGYRCTREEFFHELEHEL
QAIIIDTEQYHRALSVCERVLNRRGERYGEIRPLKIDNRGRSRCAWEGCNRVTPRRHNATDD
VVRQQLVTYFQSPLRREPGKLPMLEQAVVKLDIISKNLRGSNEAKCRNALNKRARTILRGLQ
AALPMHEDEGGSDKESWEYVEKGIVKILENAGGRNRYCREHSKAYCDRVLEGKPSEFKSSIQ
ESDIISRREQIIFNKLWRYIEARLLPLAPEGIDRIVVERTAFDLLAGKQKKIRDASSKSVESIYQY
GPRYGFTSEKEMLRKEFGGLCAYCGKPSETLLEREHILPRRLFFFDSYLNILPSCPQCNAAKGT
SLPGVSSLRIHEDAYRNYESYINELKSKRPLHFLHTEKKGILNLMRDPERAWDWERYLRLIAN
NFASIVQTQRGPRPFARYLYSKLSRRQEKPPEIAFRSGRHTALYRSVAYPDFSKAQEKAGEDG
NSHPVNHALDALLLASKLPRPEPLEARGLNHASLKAWADQVRGKAAPAGEDGIPVIPAYKCF
VPGFEETDAHGYVTVEMAMMNWNKKDTATTKQDPYGWSESRQHPTKRVSARTLFDDLRE
EKSKKNPSELIKRIYHPALRSAVEKAYQESHNRIQAAEALKNWLRDSVKNSLRSSSFSRHPSDI
RRRQDLERFANGKTDEIPVVIGIKCLDTGVAGKIDAARMDRQTGTTGHRYLTDPANRGVVL
AYPTTRSGACDRRKPCTAGIRQNYSLKTDETAFRSMPASLERGVVWGKKTHSPRAMESAFS
KELEQYLRDRRFHSYCILTAGCVVCYEDGTERFIRNFDKSKGFKKTILRNIVALKRTPLSTRV
VPLKVLTALP
>58-_10289855.9038_-_Cas9_CDS_translation
(SEQ ID NO: 6)
MMLPPRLQERLATFLPEIRVGIDFGESAGGIAVVKGNQILHAETYVDFHASDLEQRRQLRRGR
RTRHAKKMRLARLRSWVLRQKLPDDTRLPDPYVVMRDPKFHVQPGVFKTKTPGRDSATAPS
WIDLAKQGKVNASGFVRALTLIFQKRGFKWDAIELAKMTDEKMKDFLQTARVPSDNLASDI
REEIQRRRQDPDSSVRGKKKVSPDELLALLEQARERHPQPRVAEHRTVKEADLRSAVEGFGN
SINLTKATIQRWQRELSGLLNKVLRPARFENRLRTGCAWCGKPTPRKIKVRELAYEAAVRNL
RVREGRSIRPLKPEELAIFAQWWHLRGQAGESQQGGSEQKRSRKDRSQAVPKLKAIQSHLKK
IGAQEQMARQIFDLLWNEKPQGRASLCHQHLIEAAQGRTMKDVVGEWHKVKVRKAPNPCR
EQHDVRVLHRLEQILFKPGKNGPDAWRYGPAKLITLEVPKPQTEQARKGEQKLRKPESFMER
LRKETAGVCMYCDSSTHRPAEDKDHIFPQSRGGPDVWDNLVPVCRDCNMAKGDRTPFEWIG
AAGERWQRFTERVEGLAVRGVRVEREDGKEETVRISERKRALLISQDAEYPDNPTPLAHVGA
RPRQFVVALRKLFEDRGVAAPSVNFESGLPFVQRIDGRTTFQLRKSWLKKANGSDNFPKKND
WDLLNHAQDAALIAACPPHTWRDTVFRVRASRPRWDGKWTEQDGLAVSELAPDWAEYLE
RQTWPLIKVLGRYPVSWKRKFADLTFSQNPDSLDDKRLVQYLPIANMLHSGKGPDDKRHPA
ETEIVNPTLDKKFRAVATALGIKRRQTLPEKNLREEFPGIRHVKVRKQPGGRLVRVEPEDGPP
RKVEVKGASEAIVFWVKKDEPVTKLRMSIRWPTILRALNVARYEPTIPSDARILAVWRRYQL
VRFGPETGLNPGFYRVKEFDAAEVRLLPESAIPDALAQRLNLKRRNDTEESAEENKEIKLRKP
ALAKYFETLNEKDRHDPRTAS
>59-_10323148.24_-_Cas9_CDS_translation
(SEQ ID NO: 7)
MITLAIDYGASNVGIALVRNTEAGNEPLFAGTVILDARKLKEKVETRAGIRGLRRTRKTKNRR
LRELGEALSGLGMEGDKVARIVRFSNRRGYKSLFSDPNETEKVDEAESAYRCTREQFFHQLE
QELQEILSDREACDKALSVCERILNRKGDRYAEIRLIRIDNRGASRCAWGDCNKVTPRRDNAT
DDAIAQQLVTYFQSAIKTEPHKLEMLNQTVCELDSISKNLRGAIANNDDSSKKILRRRARKSL
RNLRAELPSTEPEDVSGDAWKYVEKGILNILENSGGRNRYCREHSKSYVEKVLEGKPPEFKST
IADSDIISRREQIAFSKLWRYIEARLLPLAPKGIDRIVVERTAFDLLAGKRKKIRDASSEGVENI
YQYGPMYGFPNEKEMLRKEFGGLCAYCGNPSDTLMDRDHILPRRDFFFDSYLNTLPACPTCN
SEKSASLPSQVSLRISEDAYSMYKQYLTELASKRPLHFLHTEKKGILNLMRDPARSWEVEKYL
SLIANNFASIVQTQRGPRPFARYLYSKLSTRQNKPPKIVFRSGRHTALYRGVAYPLFSKVLEKE
QNDLNGKPVNHALDAILLACKLPDSRPLEARGLNLHTLGTWRRAVMAKAPTAGEDGIPTLA
DQKHYVQGFESTDANGYVTVEMGMMNWNQKDSGTHKQDPYGWSEEKQQPTKRVAAKSL
FDELRNDKSKKKPDELIKRIYHPALRAAVEKAFGASQDRLAAAEALKKWLRDSVRNSLSSSS
FSRHPSDMRRKQDLEKFAHEDGGDIPIVIGIKCLDKGVEGKIDAERLDKQTGKTGHRYMTDP
ANRGMILAYPPTPSGEVDKRKPCTAGIRQNYALKTEDTRFASKPPELERGVVWGNNRGSLKD
LESKFEKALEKYLGECGFHSYSLLTAGCVVCYEDGTQRFIRNFDKSKDFKKAILKNIDGVRRT
PFSKRVLPLKVLTEAPAKE
>63-_10704349.1018_-_Cas9_CDS_translation
(SEQ ID NO: 8)
MATTHTLSIDFGSKYIGVALVSHSPKVPNRVLYAAVILVQPKPLNAAIKPRAAIRRIRRTRKTH
ARRLRRLAQALDGIPGADEVLRFCRRRGYSHDADPKADGEELAYAVSRNEFFAALATEIDRV
VPENHREYVLARCQEHLNAERRQTAEIRPARFENRHPTRCQWEGCAKNVARKVNATREQLS
QTLFVWLKPAFDQTHKKRPLREAIQRRIDRLVGLARGYGKDPDQETKKVLSKQKRRCFAGIL
EAVDRFADEETAQQFSENWKKTYSRQFTEILTKKQGGRLRYCRRHSHQFVDLFLAGKQPPH
GTEVHMPDLFGRSQQILFQRIWRLVAARILPLAGNRIDRVVVERVAFDILAGPFKQRTEVRED
RAAEMYWHGPMYGFGSRREMLKEEFDGRCAYCGRRRSVGEIEHLLPKSRFPFDSYFNILPAC
RECNQAKGARTPSEVGMTVHQEAYAAFADYVSKKNPPHLYHTIKKGMLKLMTRGGSMATA
QRQLGLLADNLVSITNTQKSPRPLARFLADRISRETDRPCQPDWLSGRHTALYREIVLPPEYD
KKADKRQNGLVNHAVDAIVAGCKFPSAAALENPRWSHEQKDIHLWREKVLAAATELSGGL
PKVEPIERIEFFENDLEHGYLHIDLSAFNWNRQRKSGFKQDPFGATAQGEPLKRKPADEVLAN
LLSDKDRNGQIESIAHPGLRRLLESRPEEAARLFVDWLQKTTRRGFARAKRGTHPSDKARYA
PLEAFSSMPVEDFIRKKTVVGKAKSGSPAERETIPPTIGIRCINKGVRGKLTVTRLNGADGKVQ
RFAADPQYREWYVGYRDGDDGLPVRTEPMLFAVNQGFAVKRKQGRAWVPVTDDEASILNG
VHLGARGDRKLFSSRWRSELESVFQEQGIVKWFRVTQGCYIEKTDGTGFQLRSFDDKKAWM
KNGPFENIHRVYRSPLSARR
>64-_12718451.35065_-_Cas9_CDS_translation
(SEQ ID NO: 9)
MSRVSATLHDRIREFLPTLRVGIDFGEYTGGIALVRGDAILHAETFLDFHVANLEQRRQLRRG
RRTRHARKMRLARLRSWVLRQRANGKRLPDPYCLMRDKQYMVQPGVYRTKGAPPDGSPS
WVQLAKDGKVSPAGFVRALSLIFQKRGYKWDAIALEQMSDSKLKEFLESARIPSDDLAADIK
KLIERRRLDPNDPIRGKKNRVTPEELERLLETARQREPQPRVAEHRRVKEEDLQAVVEGFGR
AANLPEETLERWKRELVGLLNKALRLPRFENRLKSGCSWCGKATPRKGKVRELAYWAAVN
NLRVRRWPAPPRPLLVGEIQPFKNWWEDRARAPEQATIKKYLQKIGTQAEMARQFHDLLKN
DSPKGRTSLCKQHLEMAARGKTMKDSGVEWQTIYVRKAPNPCREGRDARILHRLEQVLFRR
GHTGDAAWRYGPVQFITLEVPEPDTEQAPKGKQKERQEETFLARLLAEFGGKCAYAVLGGC
SGEMDKDHVFPRSREGPDVRVNLVPSCKAHNSQKGDRTPFEWLGNDPSRWRAFQESVKGL
AIAARKKEILLNETPDYPEGIPTALARIGARPROFVAALAELFRKYGVAPPRTDYQLGQPLVQ
RLQGRETDRLRRSWLKKADGSDNFPPKDPTNLSNHAEDAVLLAAAPPHTWRERIFAFRAVRP
NWKGEWVEQGGLAAPELAPDWASFQRERESPLIRVLGRYPITWKTSFADQTFGRDPTDLQA
PKLRISQPVKTLRVSQIPNIASPFWRDRFKSLADELGLAPRLTIPEDKLAERFPGLRRLQLFRQP
GGTLMTVRPEDGPARKIQFKPASEGVVVWQQEVGKKKKRLKTEISIIRPVALQRLGLPRYDPP
LPKEGRVIGRLRRHQIIWLDATPKHPPGFYRLTKFQLSGVTAVPENALPQAIARQLRLPRQEA
ENEVEEELGSITIGKAELASCCAVESRNDGGGSE
>65-_12924853.5923_-_Cas9_CDS_translation
(SEQ ID NO: 10)
MSSKLTLSIDFGYKNIGIALVQNNDGTNNPLFAGTLLYDPRQLSDKVEPRAQLRRLRRTRKTK
RNRLRKLESRLLSLGLQLEIVQKLITFCRRRGYSSLFDEPQKLDREKKDTKEEIIFPFSREEFFK
ALEKEIEILLPEDKKSKALIICEAILNRAGDPAQEIRPIRIDNRGASRCAWDGCNNVPPRRDNAL
RDALSQFIHTVYAAKLRGNNDLTQQVSGMLDRLSVLGKRRRHAGGPDPGKERKILKKAIGE
ELKLLKSISGFDPELEENDDSAPKTWSSIRRNIVNLIEQSRGRNRFCRQHSSEYVKHILEGKPIP
FKHTLSDRDLVSRREQILFQKLWRYIEARILPMAPDGIDRIIVERTAFDLLAGSRKQRQGIARK
DALEEMYQQGPRFGFKNDLEMLKMEFDGLCAYCGQQKPEMIEREHLLPQAEFFFDSYLNKV
PACKDCNQYLKQAASPGGAGLLIHEQAYEAYSRYLNKKFKDKPPHLFHTVKKGVLNLMRQP
DRIWEAERYLNLIANQFAQIVGAQRGPRPLARYLTEKLNKHYGEIPEIAFVNGRHTALWREA
AYPHFSKVREKAEGGKINHALDAIIMACDLPDLKALEAKELRPSVIPWWVKRVRNTAPPEGP
DGIPVLPKPNNMVSEFEKIHPGNFIEADLSKMNWNHKDSKVQREDSYSWSKKADIPSKRVTA
ASIIKDLRDADKKDSPESRRNEVKKIIDVVIHPQLGQVLKAANTGDTPGSNTAQALTAWLRK
AIGNNFNKTIFSAHPADQRRSKLLQEFVNGQSDGIPAFIGVKILYPWLKTNIDLNRVDPQTNNL
LHRYVADPANIGMIVAYKGNHNQVNRDRPITLEWRQSGSVIPGMKSLGQIPDGPLKGRALGE
KGISQNEWKEALHQYLANVGIAEYNIVSQGNVAVYKDGSERYIRNFSASYGFKKSLLKGIIG
VRRSPFAQKINSNVKIS
>82-_JACKQG010001031.1_-_Cas9_CDS_translation
(SEQ ID NO: 11)
MPLPSTTIAIDYGAKYIGLALVEHAEGAPNRVLYACTVVVDPKPLKELVKPRADTRRLRRTR
KTHRRRLRRLAQSLADVPNADQILRFCGRRGFSHESDDDQDEQTFRVSRARFFQSLEEEVEQ
AITPEFRERVLAACSKHLNRLRQPSAELRPARFENRGRSRCNWAGCRNNVPRAGHDIQGRLQ
QCIFLWLQPIFRESNEPERFRKSVDHWVRELAGLAKGHRRSKASTAYRKQINARKRTIYSHVR
KRVRREASEETAAQFDSNWSDYYQSNLNNVIEGKDAGRVRYCRQHSAMFVDHIMASEPIPN
REDIRDSDLISRTQQIVFRRLWRLIEGRFLPLAGGRIDRVVVERVAFDILSGPIKQRQKMPEDR
AAEMYWHGPQAGFDGRRDMLKAEFGNRCAYCGQEAFSEIEHVYNRSDFPFDSYFNIVPACT
KCNARKGGRTAFDAGMTIHDDAYAAYCDYLKAKKVLHPYHTIKKGMLNLLRRSATSERAQ
QLIGMIADNLVSITATQRAPRPLGRYLATKISERTGQRPEIAFRAGRHTALYRSVILPSYDKAE
AREADDLRNHAVDAIVLGCRLPSASALENRKWTTSRDDVLRWMESVKEAAPAMQDGLPAV
ESVVVVPHFETDAGLNYITIALSAFNWNRKRKAAHKLDPFGKTAAGIPMKRMPAATVLANL
LKGEKQRDNQIDVIAHRTLRESLLANRDTAGETFVLWLQKSVRAGLTNDEMSSHPADQERR
RLLGEFTAAPVSEIVAEPKGSKIPWTIGIRCLNRDTGAPRKVHVARRLGGNDRAQFYPAQAVI
REIRVGYREADDQLDRNAPIIFAVNQIDELSRQVPGRWEPVDVPSESPLRGRPLASEGSRKEFR
KRWEDAFADLCRKHDIAKVFRITQGCVIEKTDGAKFQIRNFDKSQPWMKGGPFKQIRRVYRS
PFQAM
>86-_PLMY01000005.1_-_Cas9_CDS_translation
(SEQ ID NO: 12)
MVTLGVDYGASSVGLALVRSDEKGHNIPLFAGTIRLDARWLKEKVEVRAGIRRLRRTKKTK
RHRLRQLEQSLTQIGLGAEQIQSIVRFSNRRGYKSLFDAGVADDDHDEAELTYRFTREEFFKA
LGTELQSIIPDTSQRQKALSTCETILNRQGDRSLEIRQIKIDNRGASRCAWEGCNRVTPRSDNA
LGEVLSQQIYTVFQSALKVNPVLCQKVETATHELHELAKRLRNASGEAASNEKKILRKRARTI
LRALKELLYTPAEGVGDAEKAWKYIETGIMNIMESRLGRNRYCREHSREYVQTICSGKLVPF
KQTISESDIISRREQIAYAKLWRYIEARILPLTPGGIDRIVVERTAFDLLAGSRKTIQKATDQFKE
EMYQHGPMYGFESVPDMLKEEFGGLCAYCGQSSSTLIDVDHILPHADFLFDSYLNILPACPKC
NSDLKGDRSVSDASLTIHPDAYRAYSDYLKKKFSTRPMHYFHSIKKGVLNLMQDASRLWEA
ERYLSLIARQFGQIVQSQRGPRPFARYLSTKLSRRQGQAPAIRFRNGRHTALYRRVAYPDFQK
QAEKAEGNVLNHALDAILLASELPDLYPVEALNLPLWQLKNWADTVRARAPKSGDEGVPIC
PDGPQYVDGFETVHPGGYVEVDLRSMRWNQKDSMTHKQDPYGFSEKTGMPTKRGSAFDLY
TKLKKEKNPSKVKSRIALIHHPALRKALSESLQSDTTGSSAAENLKTWLRISVKNSLAHSRFS
NHPGDQARRSELEKFITQEDCPIPSVIGVKMFDMGVRGKIDMKRLDRQTGGIGHRYMTQPPN
KGVIVAYPKREDGKPDLTKPCCVYHKQDLSMTPENISIFKPPPPILSDGVILGKKPYDKGDRR
KALEKYLSDCGFHSYVYLTPGCTIRYKDGNEWFVRNFDSSEDFKKGRLREIIGTRRTPFVDSLI
PLKVLSQ
>Cas9 IID 88
(SEQ ID NO: 13)
>MSIPAQLSAAVQTFLPTLRLGLDLGERAVGIAVVRGNEVLHAETVIDFHEATLKERRRLRRG
RRTRRAKKSRIARLRSWILRQIVNGKRLPDPYILMRQKRFQCQPGEYRQKVQLAKSALPSWV
EAVKQGRETSDEAFVIALTHLFQKRGYRWGGSDVQAMDDNTLADELRKIRLTPAVAEQVRR
EVERRKNDPNAPKGFTGKINGIEQLIEQALNRRRQPRVAEHRSIVEDEVRAVVTSFGRHHGIA
EDTMTRWRAELVCLLNKPVRAARFENRALTGCTWCGAHTPKKSRPEVQELAYWAAVANV
RVAAGRQPRPLTQSERAQFVEWWNADAQRRPTQPAIKRYLTSIGAQEEMARQFADLLNRRN
LNGRTNLCLAHLREQAEGAFFCPQHQGVCRSAPNGQHRAVESARSRESSASRVWNPARAW
HDRRVVARIERMLFMRDGTPRYGGIPSLITIEVPKPDTAHRYECPHCHEALAVNLRVRYRITK
LELKPTKVRQNEAAFTCPQCRKPFEINGKRKIGTPNGLKPINVKLGLTHAVVWWAGGGKKA
RHVADTNGQCIYCGTNVDVGSVKLDHIFPQSMAGPGIYMNMVAACERCNNEKYNRTPWQ
WKGHDQAWWQAFEARLDRLFLPMRKREMLLSREASYPENPTALARVGGRAREFMRELQV
MFARHAIPSERIVTGYRHDADIVMQMIEGWMTDRLRRSWMSGVDGRENFLPKDRADLRNH
AQDAVLVAACPPHTWRERIFCYGPDRDMALPDLAPNWRNYESGMRDRHPLVVPLGRYRIR
WRKQFLDQTFWKQPLGRKPVVYRQLAELKKSDASSIKDERIRCAFLSVCQAYNIGSEKTLTE
DAQADLQNRLVSLGMVTPVRRVQCFSQKGGLPISVRPHDGPVRITQVKPTSDGVVLWLPAG
VRLETARTRDLKISVIRPKPVVGWPSPDVPGQAVSELDPPVPPEAQRIATWYRYQCVRFSPHD
GWYRLKEFSEKKLTVMPAIRLPKKLRNDAGGHDGEETGNDEREFGKEALLAAVRANAMAT
FCDPFD

### RNA Guide Scaffold Sequences:

>25-sgRNA
(SEQ ID NO: 14)
GTTTCAATCAACCGATAGAAATATCGGTCCGGTTGAAAAGAGCATCGGTC
TGAAGGATGCACTCCGGGATAGGGCAGTCCCGGCTCTTGCTGTTTCCCCG
GTAAGACCTCGGAAGCAAGTCCTTCAGCAAGTCGAAAGACACGATGTGAG
CCTATTTT
>37-_sgRNA
(SEQ ID NO: 15)
GTTTTAGTCGAAAGGCTGGAGCGTTCTTTGGCAATTCGACCATGTTTCCA
ACAGGTTGCGGCTAAAAAGAGCCACGACCGCACCTCGGCACCCTGGGATC
GGACGGACAGTCCCCGGCCCTGCTGCTCGGATGCTTCATGCGCCCGACAA
ACGACGCGGTCTATGTGGTGAGTCCGTATTTTTT
>42_sgRNA
(SEQ ID NO: 16)
GTTTCAATCAAACTGATTTGAAAAAATCAGTTCCGGTTGAAAAGAGCATC
CGTCTGAAGGGCACTCCGGGATAGGGCAGTCCCGGCTCTTGCTGTTTCCC
TGGCACATCACTGTGCTCCGGAAATGAACCCTCGGACAAGTCGAAAGACA
GGATGTGAGCCTAATTTATTT
>43-sgRNA
(SEQ ID NO: 17)
GTTTCAGGCGACACGAAGAAATTCGTGTGGTGTCTGAAAAGAGCCACGTC
CAGGAGTCCGGCACCCGTGGATTGGACAGTCTACGGCCCTGCACGGCCGG
TATAGAGAAGCCGGCCAAACGGCCCTGGCCAATGTGGTGAGTCCATTTTT
>49-sgRNA
(SEQ ID NO: 18)
GTTTCAATCAAGCTGAAGAAATTCAGCTCCGGTTGAAAAGAGCATCCGTC
TGGTAGCCATGCACTCCGGAATGGGGCAGTTCCGGCTCTTGCGACTTAGT
GGGTGAAAGCTCATTGAGCCAACTACGAGACACGTCTCTACGAGACAGGA
TGTGAGCCCTTATTT
>58-_sgRNA
(SEQ ID NO: 19)
CTTTCACTCTAGCGAAAGCTAGAGTGAAAGAAGCCCAGGCGCTGCTCCAG
TCCTCGCCGATGTAACCCAGCATCGGAACCAGGGTGTGGGCATCCCCGTA
GGCCGGTACTCGGACCCCGGCAAAGGGCAAGGGTTGGTGGCGCACTCGAC
GAGCGAGGAGCGCCACCTCATTTT
>59-_sgRNA
(SEQ ID NO: 20)
GTTTCAATCAAGCTGAGAAATCAGCTCCGGTTGAAAAGAGCATCCGTCTG
GCGGGCACTCCGGAATGGGGCAGTTCCGGCTCTTGCAGTCCAGTGAGTTC
ATTCGCATTGAACCAACTGCCAGGCACGTCTTCGGACAGGATGTGAGCCC
GTATTTCTT
>63-sgRNA
(SEQ ID NO: 21)
GTTTTGGTCAGCCGAAAGGTTGCGGCCAAAAAGAGCCACGACCGCTATTC
GGCACCCGGGGATCGGACGGACAGTCCCCGGCCCTGCGGATCGGGCGTTT
CGCACGTCCGACAAACGACGCGGTCCATGTGGTGAGTCCGTTTTATTTTT
T
>64-sgRNA
(SEQ ID NO: 22)
CTTTCACTCTAGCGAAAGCTAGAGTGAAAGAAGCCCAGGCGCCTCCGTTC
TCCTCGCTGTAACCCAGCAGCGGGTTTAAGGCATAGGAGGCCCCGCTTCC
AGGCTCTCGGCTCGCCTGGAACTCCCGCAAGGGAGAAGGGCAAGGGTCGC
GACGCCGCTGGCCGGGCAGAAGGCGTCGCCAAATCGCTCTTT
>65-_sgRNA
(SEQ ID NO: 23)
GTTTCAGTCACCCTGAACGAAAGTTCGGGTTCAGGCTGAAAAGAGCATCT
GTCCGGAAGGGCCACTCCGGGTTAGGGCAGATCCGGCACTTGGGCCTCCT
TGGTCTTTTTGACTCTGGAGGAAACCTTTCGGCGAGTCTTCGGACAAGAT
GCGAGCC
>82-_gRNA
(SEQ ID NO: 24)
GTTTCAGGCAACATCGAAAGATGTCGCGTCTGAAAAGAGCCTAGACGCGA
GCCAGCACCCAGGAATGCGGACAGTTCCTGGCCCTGCATGACGGTCATAG
AGAAGACTGTCGAAATGGCTCTCGTCCATAATGGTGAGTCCATATTATTT
T
>86_sgRNA
(SEQ ID NO: 25)
GTTTCAATCAAACTGATGAAAATCAGTTCCGGTTGAAAAGAGCATCCGTC
CGGAGGGTGCACTCCGGGATGGGGCAGTCCCGGCACTTGCGTTTTCCCCG
GCTTACGCTTCGGAAAAAGGCCCTTCGGCACGTCGAAAGACAGGATGTGAG
CCCAATTTAAATATTGTT
>88_sgRNA
(SEQ ID NO: 26)
CTTTCAGAATGCTCGAAAGAGCATTCTGAAAGAAGCCCAGACGCCTCGCG
CGCCCGCAGGTAGCCCAGCTTGCGGCGTCTGTCGTATCGGTGGGGTGAGA
CAGACATTAACCGCACCCACCTGCAAATAGGCGAGGCCTCACCGGGGAGG
TTCGGCGGACCCCGGCGCCACTGGCGAGGGCAAGGGTAGAGAACGCTGCA
TCCGAACCTCTGCAGCGTTCGCTTTT

### Engineered Single Guide RNA Sequences:

>25-sgRNA (SEQ ID NO:759) (n)ₓGTTTCAATCAACCGATAGAAATATCGGTCCGGTTGAAAAGAGCATCGGTCTGAAGGA TGCACTCCGGGATAGGGCAGTCCCGGCTCTTGCTGTTTCCCCGGTAAGACCTCGGAAGCA AGTCCTTCAGCAAGTCGAAAGACACGATGTGAGCCTATTTT; wherein (n)x is the target sequence, n is natural or modified nucleotide, and x is 18-25

>37-_sgRNA (SEQ ID NO:760) (n)ₓGTTTTAGTCGAAAGGCTGGAGCGTTCTTTGGCAATTCGACCATGTTTCCAACAGGTTG CGGCTAAAAAGAGCCACGACCGCACCTCGGCACCCTGGGATCGGACGGACAGTCCCCGG CCCTGCTGCTCGGATGCTTCATGCGCCCGACAAACGACGCGGTCTATGTGGTGAGTCCGT ATTTTTT; wherein (n)x is the target sequence, n is natural or modified nucleotide, and x is 18-25

>42_sgRNA (SEQ ID NO:761) (n)ₓGTTTCAATCAAACTGATTTGAAAAAATCAGTTCCGGTTGAAAAGAGCATCCGTCTGA AGGGCACTCCGGGATAGGGCAGTCCCGGCTCTTGCTGTTTCCCTGGCACATCACTGTGCT CCGGAAATGAACCCTCGGACAAGTCGAAAGACAGGATGTGAGCCTAATTTATTT; wherein (n)x is the target sequence, n is natural or modified nucleotide, and x is 18-25

>43-sgRNA (SEQ ID NO:762) (n)ₓGTTTCAGGCGACACGAAGAAATTCGTGTGGTGTCTGAAAAGAGCCACGTCCAGGAGT CCGGCACCCGTGGATTGGACAGTCTACGGCCCTGCACGGCCGGTATAGAGAAGCCGGCC AAACGGCCCTGGCCAATGTGGTGAGTCCATTTTT; wherein (n)x is the target sequence, n is natural or modified nucleotide, and x is 18-25

>49-sgRNA (SEQ ID NO:763) (n)ₓGTTTCAATCAAGCTGAAGAAATTCAGCTCCGGTTGAAAAGAGCATCCGTCTGGTAGC CATGCACTCCGGAATGGGGCAGTTCCGGCTCTTGCGACTTAGTGGGTGAAAGCTCATTGA GCCAACTACGAGACACGTCTCTACGAGACAGGATGTGAGCCCTTATTT; wherein (n)x is the target sequence, n is natural or modified nucleotide, and x is 18-25

>58-_sgRNA (SEQ ID NO:764) (n)ₓCTTTCACTCTAGCGAAAGCTAGAGTGAAAGAAGCCCAGGCGCTGCTCCAGTCCTCGC CGATGTAACCCAGCATCGGAACCAGGGTGTGGGCATCCCCGTAGGCCGGTACTCGGACC CCGGCAAAGGGCAAGGGTTGGTGGCGCACTCGACGAGCGAGGAGCGCCACCTCATTTT; wherein (n)x is the target sequence, n is natural or modified nucleotide, and x is 18-25

>59-_sgRNA (SEQ ID NO:765) (n)ₓGTTTCAATCAAGCTGAGAAATCAGCTCCGGTTGAAAAGAGCATCCGTCTGGCGGGCA CTCCGGAATGGGGCAGTTCCGGCTCTTGCAGTCCAGTGAGTTCATTCGCATTGAACCAAC TGCCAGGCACGTCTTCGGACAGGATGTGAGCCCGTATTTCTT; wherein (n)x is the target sequence, n is natural or modified nucleotide, and x is 18-25

>63-sgRNA (SEQ ID NO:766) (n)ₓGTTTTGGTCAGCCGAAAGGTTGCGGCCAAAAAGAGCCACGACCGCTATTCGGCACCC GGGGATCGGACGGACAGTCCCCGGCCCTGCGGATCGGGCGTTTCGCACGTCCGACAAAC GACGCGGTCCATGTGGTGAGTCCGTTTTATTTTTT; wherein (n)x is the target sequence, n is natural or modified nucleotide, and x is 18-25

>64-sgRNA (SEQ ID NO:767) (n)ₓCTTTCACTCTAGCGAAAGCTAGAGTGAAAGAAGCCCAGGCGCCTCCGTTCTCCTCGCT GTAACCCAGCAGCGGGTTTAAGGCATAGGAGGCCCCGCTTCCAGGCTCTCGGCTCGCCTG GAACTCCCGCAAGGGAGAAGGGCAAGGGTCGCGACGCCGCTGGCCGGGCAGAAGGCGT CGCCAAATCGCTCTTT; wherein (n)x is the target sequence, n is natural or modified nucleotide, and x is 18-25

>65-_sgRNA (SEQ ID NO:768) (n)xGTTTCAGTCACCCTGAACGAAAGTTCGGGTTCAGGCTGAAAAGAGCATCTGTCCGGA AGGGCCACTCCGGGTTAGGGCAGATCCGGCACTTGGGCCTCCTTGGTCTTTTTGACTCTG GAGGAAACCTTTCGGCGAGTCTTCGGACAAGATGCGAGCC; wherein (n)x is the target sequence, n is natural or modified nucleotide, and x is 18-25

>82-_sgRNA (SEQ ID NO:769) (n)ₓGTTTCAGGCAACATCGAAAGATGTCGCGTCTGAAAAGAGCCTAGACGCGAGCCAGCA CCCAGGAATGCGGACAGTTCCTGGCCCTGCATGACGGTCATAGAGAAGACTGTCGAAAT GGCTCTCGTCCATAATGGTGAGTCCATATTATTTT; wherein (n)x is the target sequence, n is natural or modified nucleotide, and x is 18-25

>86_sgRNA (SEQ ID NO:770) (n)ₓGTTTCAATCAAACTGATGAAAATCAGTTCCGGTTGAAAAGAGCATCCGTCCGGAGGG TGCACTCCGGGATGGGGCAGTCCCGGCACTTGCGTTTTCCCCGGCTTACGCTTCGGAAAA AGGCCCTTCGGCACGTCGAAAGACAGGATGTGAGCCCAATTTAAATATTGTT; wherein (n)x is the target sequence, n is natural or modified nucleotide, and x is 18-25

>88_sgRNA (SEQ ID NO:771) (n)ₓCTTTCAGAATGCTCGAAAGAGCATTCTGAAAGAAGCCCAGACGCCTCGCGCGCCCGC AGGTAGCCCAGCTTGCGGCGTCTGTCGTATCGGTGGGGTGAGACAGACATTAACCGCAC CCACCTGCAAATAGGCGAGGCCTCACCGGGGAGGTTCGGCGGACCCCGGCGCCACTGGC GAGGGCAAGGGTAGAGAACGCTGCATCCGAACCTCTGCAGCGTTCGCTTTT; wherein (n)x is the target sequence, n is natural or modified nucleotide, and x is 18-25

>Cas9 IID 41
(SEQ ID NO: 27)
MENFTLAIDFGSVNIGLALVCNRDGVNEPLFAGIITYSNEVLKKKSNPRA
QIRRLRRTRKTKKSRLNRLRQALFSLGLDQDVVNALVHFCRRRGYKALYG
ADREAEENGQAEETVFRYSREEFFRALNKEIDRLLPQDLRPPVLKVCDRV
LNRHGELNREIRPIRIDNRGASRCAWSECDKVPPRRKNAIRDALAQFVYA
IIDLPRLRNDENLHRDLDAALDTVAELAKRLRSVNGHDPDREKKVLRKRI
REALKPVKDLAALDTWKVNSENIMNLLEKSQGRNRYCREHSGEYVRCLLE
GRTVPFKASLVERDMVSRREEILYQKLWRYIETRILPLAPGGIDRLVVER
VAFDLLAGTRKQRQKVGDKTVEESYQFGPRHGFKSDLEMLRQEFDGLCAY
CGKPSGQIIQREHILPRGDFLFDSYLNILPSCPACNQVIKAKASPQAAGL
HVHESAYQAYCRYLAGKFKIRPPHEYHTIKKGLLNLMRQPDRTWEAEQYL
ALIAGHFTQVAQTQRAPRPMARFLCERLRQRFGRSPQLAFRNGRHTDLWR
RAAYPDFDKLKEKEEGGLINHALDALVLACDLPGMTALEGLNLKPKELKS
WVRAVTVAAPPPGPDGVPVAPEPGRAVPGFEEILPGNYLRADLTFFNWNR
KDLGVQSQDIYGWSKNENVPTKRKTALDLVAELRKQTNTSGVKKIIDTVA
HPNLRQALQAANTGDKPGEQAVQALIDWLRRAIKPALDKARFSDHPGDQA
RRKALHDFVSGNLDGPPVTIGVKRLYPYGSGKTDLQRIDPKTGRLVHRYI
ADPANKALIAAYPLKKGRIRRDSPIILEVRQNDALKPQGKTLARPASGPL
AGRGLGRPAPDPDQWKAELHRCLAEAGVAEYAVVAQGCVVKYEDGSEKYI
RNFSTSQGFKNGFLKNIVAVRRSPFAAAETANVRLF

>41_sgRNA (SEQ ID NO:28) (n)xGTTTCAGTTACCCTGAACGAAAGTTCAGGCTGAAGCTGAAAAGAGCATCCGTCCGGA AGGTCCACTCCGGGTTAGGGCAGATCCGGCTCTTGGTCCTCTCCTGGCCCTTTTCGGGCTC CGAGAGGAAGCCTTCCGGCATGTCTTCGGACAGGATGTGA; wherein (n)x is the target sequence, n is natural or modified nucleotide, and x is 18-25

TABLE 1
Activities of Cas9 IID proteins in Mammalian
Cells are shown below in Table 1:

| | Activity, 72 h | Protein size | |
|---|---|---|---|
| Gene# | tfx* | (AA) | Putative PAM |
| 25 | ++++ | 944 | NACC |
| 37 | + | 960 | NACC |
| 42 | + | 942 | NACC |
| 43 | + | 938 | NVC |
| 49 | ++ | 949 | NVC |
| 58 | + | 1007 | NRGM |
| 59 | ++ | 955 | NAC |
| 63 | ++ | 948 | NVCCC |
| 64 | + | 960 | NGG |
| 65 | + | 958 | NAV |
| 82 | + | 935 | NVC |
| 86 | + | 946 | NAC |

| | |
|---|---|
| Activity | Description: at least 1 guide indel % |
| + | Above background, <1% |
| ++ | 1% < IID < 2% |
| +++ | 2% < IID < 10% |
| ++++ | >10% |

### Example 2: Protein Mutagenesis to Improve the On-Target Activity of Cas9-IID

To enhance the activity of Cas9-IID, an array of variants was designed and evaluated using WT Cas9-IID-25 (SEQ ID NO: 1) as the foundation. These variants were predominantly comprised of single amino acid substitutions with positively charged residues. The work revealed a large panel of point mutations sequences that can enhance the on-target activity of Cas9-IID in human cells, which allows further engineering of this nuclease on top of the current iteration (see e.g., Tables 2A-2C).

TABLE 2A
Point mutations of Cas9-IID-25 (SEQ. ID. NO: 1) with enhanced
editing efficiency in human cells. The effect of point mutations
was evaluated by measuring the editing efficiency in HEK293-FT
cells over two genomic targets. The editing efficiency of
each mutation was normalized to the Wild-type nuclease, and
reported as the relative activity cutoff of 1.2.

| | Relative activity - | Relative activity - |
|---|---|---|
| Mutant | Target 1 | Target 2 |
| L19R | 1.15 | 1.21 |
| H70K | 1.66 | 1.25 |
| H70R | 1.53 | 0.98 |
| A71K | 1.76 | 1.36 |
| A71R | 1.47 | 1.17 |
| E131R | 1.24 | 1.09 |
| H153K | 1.27 | 1.15 |
| A180R | 1.96 | 1.25 |
| L185R | 1.7 | 0.93 |
| N221R | 1.33 | 0.94 |
| S239K | 1.79 | 1.34 |
| I302V | 1.15 | 1.21 |
| S366K | 1.84 | 1.14 |
| W367R | 1.43 | 1.18 |
| I370R | 1.51 | 0.87 |
| D372K | 1.93 | 1.58 |
| A376K | 1.52 | 1.18 |
| L410K | 1.26 | 1 |
| S473K | 1.42 | 1.17 |
| T537K | 1.44 | 1.21 |
| L538R | 1.21 | 0.97 |
| N558R | 1.34 | 1.01 |
| A571K | 1.31 | 1.08 |
| S609R | 1.36 | 1.05 |
| S626V | 1.34 | 0.99 |
| T688K | 1.25 | 1.16 |
| E689K | 2.14 | 1.16 |
| N700K | 1.56 | 1.19 |
| C736R | 1.52 | 1.23 |
| N737K | 1.21 | 0.97 |
| Q786R | 2.27 | 1.47 |
| A821R | 1.28 | 1 |
| D827K | 1.76 | 1.07 |
| I843K | 3.45 | 1.61 |
| I843R | 3.03 | 1.48 |
| L847K | 1.12 | 1.22 |
| A848R | 2.1 | 1.32 |
| L853K | 2.05 | 1.41 |
| Q913K | 4.08 | 1.35 |
| Q913S | 1.52 | 1.13 |
| F920K | 1.24 | 1.2 |
| L363Ins | 2.69 | 0.66 |
| K694Ins | 1.74 | 1.51 |

TABLE 2B
Point mutations of Cas9-IID-25 (SEQ ID NO: 1) with enhanced
editing efficiency in human cells. The effect of point mutations
was evaluated by measuring the editing efficiency in HEK293-FT
cells over two genomic targets. The editing efficiency of each
mutation was normalized to the Wild-type nuclease.

| | Relative activity - | Relative activity - |
|---|---|---|
| Mutant | Target 1 | Target 2 |
| I13V | 1.03 | 0.96 |
| L19R | 1.15 | 1.21 |
| H40R | 0.53 | 0.58 |
| H70K | 1.66 | 1.25 |
| H70R | 1.53 | 0.98 |
| A71K | 1.76 | 1.36 |
| A71R | 1.47 | 1.17 |
| H153K | 1.27 | 1.15 |
| A180R | 1.96 | 1.25 |
| L185R | 1.7 | 0.93 |
| N221R | 1.33 | 0.94 |
| S239K | 1.79 | 1.34 |
| I302V | 1.15 | 1.21 |
| S366K | 1.84 | 1.14 |
| W367R | 1.43 | 1.18 |
| D372K | 1.93 | 1.58 |
| S473K | 1.42 | 1.17 |
| T537K | 1.44 | 1.21 |
| L538R | 1.21 | 0.97 |
| N558R | 1.34 | 1.01 |
| S609R | 1.36 | 1.05 |
| S609V | 1.1 | 0.99 |
| A610R | 1.09 | 0.95 |
| S626V | 1.34 | 0.99 |
| T688K | 1.25 | 1.16 |
| E689K | 2.14 | 1.16 |
| N700K | 1.56 | 1.19 |
| C736R | 1.52 | 1.23 |
| Q786R | 2.27 | 1.47 |
| A821R | 1.28 | 1 |
| D827K | 1.76 | 1.07 |
| I843K | 3.45 | 1.61 |
| I843R | 3.03 | 1.48 |
| L847K | 1.12 | 1.22 |
| A848R | 2.1 | 1.32 |
| L853K | 2.05 | 1.41 |
| Q913K | 4.08 | 1.35 |
| Q913S | 1.52 | 1.13 |
| S914N | 0.46 | 0.77 |
| F920K | 1.24 | 1.2 |
| F920S | 0.93 | 0.94 |
| I928V | 1.12 | 1.04 |
| V942K | 1.07 | 1.06 |
| A695R | 0.94 | 0.75 |
| L363Ins | 2.69 | 0.66 |
| K694Ins | 1.74 | 1.51 |

TABLE 2C
Point mutations of Cas9-IID-25 (SEQ ID NO: 1), editing efficiency in human
cells. The effect of point mutations was evaluated by measuring the editing
efficiency in HEK293-FT cells over two genomic targets. The editing efficiency
of each mutation was normalized to the Wild-type nuclease, and mutations
can be selected with a relative activity greater than or equal to 1.2 for
at least one of the genomic targets (see e.g., Table 2A).

| | Genomic target 1 | | | | | | | |
|---|---|---|---|---|---|---|---|---|
| | BioRep1 | | | BioRep2 | | | | Relative |
| Variants | Rep1 | Rep2 | Rep3 | Rep1 | Rep2 | Rep3 | Average | activity |
| D7A | 0.02 | 0.03 | 0.05 | 0.02 | 0.03 | 0.05 | 0.03 ± 0.01 | 0.01 |
| I13V | 4.22 | 3.57 | 3.74 | 4.32 | 3.49 | 3.61 | 3.83 ± 0.36 | 1.03 |
| L19R | 4.83 | 4.85 | 4.91 | 3.88 | 3.85 | 3.47 | 4.3 ± 0.64 | 1.15 |
| T20N | 3.99 | 2.71 | 3.39 | 4.14 | 4.29 | 3.89 | 3.74 ± 0.59 | 1 |
| H40R | 1.86 | 2.15 | 1.74 | 2.03 | 2.4 | 1.62 | 1.97 ± 0.29 | 0.53 |
| Q69K | 4.03 | 3.66 | 3.34 | 4.07 | 3.88 | 3.35 | 3.72 ± 0.33 | 1 |
| H70K | 6.14 | 5.73 | 6.25 | 7.33 | 5.35 | 6.39 | 6.2 ± 0.67 | 1.66 |
| H70R | 6.05 | 5.9 | 4.83 | 5.83 | 5.54 | 6.16 | 5.72 ± 0.48 | 1.53 |
| A71K | 8.59 | 5.46 | 5.57 | 6.65 | 6.91 | 6.17 | 6.56 ± 1.15 | 1.76 |
| A71R | 6.96 | 5.14 | 5.73 | 5.56 | 5.03 | 4.38 | 5.47 ± 0.87 | 1.47 |
| S89C | 5.26 | 3.29 | 3.75 | 4.29 | 4.06 | 3.4 | 4.01 ± 0.72 | 1.07 |
| E105K | 3.23 | 2.38 | 2.23 | 2.76 | 2.17 | 2.32 | 2.52 ± 0.41 | 0.67 |
| T106K | 4.69 | 3.91 | 4.24 | 4.14 | 4.58 | 4.01 | 4.26 ± 0.31 | 1.14 |
| E131R | 4.53 | 4.45 | 5.24 | 4.82 | 4.38 | 4.39 | 4.64 ± 0.34 | 1.24 |
| H153K | 5.85 | 3.85 | 5.19 | 4.88 | 4.76 | 3.79 | 4.72 ± 0.79 | 1.27 |
| A180R | 8 | 5.5 | 7.14 | 7.98 | 7.65 | 7.58 | 7.31 ± 0.94 | 1.96 |
| L185R | 9.64 | 4.32 | 5.68 | 6.74 | 5.76 | 5.95 | 6.35 ± 1.79 | 1.7 |
| I215V | 5.24 | | 4.29 | 4.03 | 4.01 | 3.61 | 4.24 ± 0.61 | 1.14 |
| N221R | 6.97 | 4.37 | 6.29 | 4.01 | 3.83 | 4.26 | 4.96 ± 1.33 | 1.33 |
| S239K | 8.61 | 5.76 | 6.57 | 6.32 | 6.76 | 6.14 | 6.69 ± 1 | 1.79 |
| I302V | 5.06 | 3.06 | 4.98 | 4.16 | 4.6 | 3.83 | 4.28 ± 0.76 | 1.15 |
| S366K | 8.29 | 6.32 | 8.14 | 6.1 | 6.09 | 6.19 | 6.86 ± 1.06 | 1.84 |
| W367R | 7.58 | 4.65 | 5.05 | 4.93 | 4.95 | 4.86 | 5.34 ± 1.11 | 1.43 |
| I370R | 7.32 | 4.25 | 5.44 | 5.48 | 5.63 | 5.66 | 5.63 ± 0.98 | 1.51 |
| D372K | 10.17 | 5.27 | 6.36 | 7.18 | 7.26 | 6.86 | 7.18 ± 1.64 | 1.93 |
| A376K | 7.26 | 5.65 | 6.28 | 5.22 | 4.39 | 5.2 | 5.67 ± 1 | 1.52 |
| M387R | 5.1 | | 4.36 | 3.64 | 4.12 | 3.87 | 4.22 ± 0.56 | 1.13 |
| L410K | 6.49 | 3.1 | 5.23 | 4.44 | 4.71 | 4.32 | 4.72 ± 1.12 | 1.26 |
| T458R | 3.3 | 2.59 | 3.16 | 2.68 | 3.4 | 3 | 3.02 ± 0.33 | 0.81 |
| H469K | 4.91 | 3.03 | 4.75 | 4.29 | 4.36 | 4.54 | 4.31 ± 0.67 | 1.16 |
| S473K | 7.97 | | 5.53 | 4.64 | 4.28 | 4.12 | 5.31 ± 1.59 | 1.42 |
| K495R | 4.11 | 2.96 | 3.84 | 3.9 | 4.02 | 3.75 | 3.76 ± 0.41 | 1.01 |
| T537K | 7.84 | 4.63 | 5.72 | 4.59 | 4.66 | 4.71 | 5.36 ± 1.29 | 1.44 |
| L538R | 6.73 | 3.71 | 4.82 | 3.56 | 4.21 | 4 | 4.51 ± 1.18 | 1.21 |
| H552A | 3.96 | 2.54 | 4.46 | 3.15 | 2.96 | 3.28 | 3.39 ± 0.7 | 0.91 |
| N558R | 8.09 | 3.72 | 4.43 | 4.46 | 4.97 | 4.43 | 5.02 ± 1.56 | 1.34 |
| A571K | 4.55 | 3.81 | 5.44 | 5.06 | 5.18 | 5.27 | 4.89 ± 0.61 | 1.31 |
| N577A | 0.06 | | 0.05 | 0.07 | 0.11 | 0.08 | 0.07 ± 0.02 | 0.02 |
| H578A | 0.06 | 0.03 | 0.05 | 0.07 | 0.03 | 0.05 | 0.05 ± 0.02 | 0.01 |
| E588K | 4.74 | 2.41 | 3.57 | 3.75 | 3.89 | 3.45 | 3.64 ± 0.75 | 0.97 |
| L598R | 4.5 | 2.93 | 4.09 | 4.49 | 4.27 | 3.77 | 4.01 ± 0.59 | 1.07 |
| S609R | 7.03 | 4.47 | 4.55 | 4.93 | 4.92 | 4.44 | 5.06 ± 0.99 | 1.36 |
| S609V | 4.69 | 3.17 | 4.53 | 5.05 | 4.21 | 2.87 | 4.09 ± 0.87 | 1.1 |
| A610R | 4.66 | 2.99 | 3.69 | 4.42 | 4.17 | 4.58 | 4.09 ± 0.64 | 1.09 |
| S611K | 3.61 | 3.12 | 4.01 | 4.51 | 3.53 | 4.01 | 3.8 ± 0.48 | 1.02 |
| A621K | 5.03 | | 3.65 | 4.37 | 4.03 | 4.2 | 4.26 ± 0.51 | 1.14 |
| S626V | 6.63 | 4.64 | 4.32 | 5.03 | 4.75 | 4.59 | 4.99 ± 0.83 | 1.34 |
| Q657R | 3.65 | 2.12 | 3.07 | 3.46 | 3.45 | 3.69 | 3.24 ± 0.59 | 0.87 |
| T688K | 6.94 | | 3.53 | 5.16 | 4.58 | 3.08 | 4.66 ± 1.52 | 1.25 |
| E689K | 8.58 | 7.67 | 7.34 | 8.97 | 7.72 | 7.53 | 7.97 ± 0.65 | 2.14 |
| N700K | 6.92 | 3.07 | 7.33 | 6.89 | 6.41 | 4.35 | 5.83 ± 1.71 | 1.56 |
| C736R | 5.98 | 3.91 | 6.35 | 6.48 | 6.18 | 5.21 | 5.69 ± 0.98 | 1.52 |
| N737K | 6.14 | 3.18 | 4.25 | 4.63 | 4.74 | 4.17 | 4.52 ± 0.97 | 1.21 |
| H747R | 4.4 | | 4.3 | 3.65 | 3.84 | 3.99 | 4.04 ± 0.31 | 1.08 |
| N750R | 5.6 | 2.67 | 4.62 | 2.96 | 2.89 | 3.21 | 3.66 ± 1.18 | 0.98 |
| Q786R | 11.62 | 6.36 | 7.38 | 7.83 | 9.03 | 8.5 | 8.45 ± 1.8 | 2.27 |
| N800R | 5.98 | 3.16 | 3.66 | 5.08 | 4.38 | 3.64 | 4.32 ± 1.06 | 1.16 |
| A821R | 5.78 | 4.47 | 4.83 | 4.02 | 4.53 | 4.94 | 4.76 ± 0.59 | 1.28 |
| D827K | 9.68 | 5.31 | 6.9 | 6.13 | 5.6 | 5.71 | 6.56 ± 1.63 | 1.76 |
| M828R | 4.18 | 3.3 | 4.35 | 4.85 | 4.66 | 3.98 | 4.22 ± 0.55 | 1.13 |
| I843K | 17.69 | 7.31 | 14.01 | 12.86 | 13.36 | 12 | 12.87 ± 3.36 | 3.45 |
| I843R | 14.5 | 8.61 | 12.12 | 11.99 | 10.19 | 10.38 | 11.3 ± 2.03 | 3.03 |
| L847K | 5.98 | 3.44 | 3.99 | 4.4 | 4.01 | 3.3 | 4.19 ± 0.97 | 1.12 |
| A848R | 10.62 | 5.78 | 6.46 | 8.65 | 8.24 | 7.3 | 7.84 ± 1.73 | 2.1 |
| L853K | 11.19 | 4.34 | 7.74 | 7.43 | 7.09 | 8.02 | 7.64 ± 2.19 | 2.05 |
| H866K | 6.07 | 3.21 | 4.45 | 4.39 | 4.51 | 3.78 | 4.4 ± 0.96 | 1.18 |
| L873W | 5.18 | 2.16 | 4.09 | 4.64 | 3.51 | 3.65 | 3.87 ± 1.04 | 1.04 |
| A901E | 5.09 | 3.65 | 4.5 | 4.59 | 4.1 | 4.05 | 4.33 ± 0.5 | 1.16 |
| A901K | 5.27 | 2.75 | 4.91 | 4.9 | 4.31 | 4.04 | 4.36 ± 0.91 | 1.17 |
| V908I | 4.96 | 3.17 | 5.22 | 4.49 | 3.99 | 3.34 | 4.2 ± 0.84 | 1.12 |
| R909A | 0.34 | | 0.12 | 0.22 | 0.18 | 0.12 | 0.2 ± 0.09 | 0.05 |
| N910A | 0.33 | 0.23 | 0.29 | 0.38 | 0.26 | 0.29 | 0.3 ± 0.05 | 0.08 |
| F911A | 0.34 | 0.25 | 0.26 | 0.32 | 0.28 | 0.26 | 0.29 ± 0.04 | 0.08 |
| D912S | 1.32 | 0.6 | 0.82 | 1.13 | 0.86 | 1.03 | 0.96 ± 0.25 | 0.26 |
| Q913K | 15.69 | 10.97 | 16.65 | 17.63 | 14.07 | 16.39 | 15.23 ± 2.4 | 4.08 |
| Q913S | 7.11 | 4.13 | 5.73 | 5.98 | 5.68 | 5.31 | 5.66 ± 0.97 | 1.52 |
| S914N | 1.9 | 1.26 | 1.78 | 1.74 | 2.11 | 1.55 | 1.72 ± 0.29 | 0.46 |
| K915Y | 1.01 | 0.63 | 0.77 | 0.97 | 0.96 | 0.8 | 0.86 ± 0.15 | 0.23 |
| D916G | 6.6 | 1.04 | 4.72 | 4.63 | 4.34 | 4.19 | 4.25 ± 1.8 | 1.14 |
| K918R | 3.53 | 1.55 | 2.14 | 2.99 | 2.21 | 2.08 | 2.42 ± 0.71 | 0.65 |
| F920K | 5.82 | 4.21 | 3.79 | 4.17 | 4.99 | 4.69 | 4.61 ± 0.73 | 1.24 |
| F920S | 2.34 | 3.56 | 3.73 | 4.07 | 3.44 | 3.64 | 3.46 ± 0.59 | 0.93 |
| R921I | 0.99 | 0.99 | 1.12 | 1.24 | 1.12 | 0.98 | 1.07 ± 0.1 | 0.29 |
| I928V | 5.52 | 2.43 | 4.38 | 3.99 | 4.48 | 4.37 | 4.2 ± 1.01 | 1.12 |
| K930R | 1.53 | 3.04 | 3.41 | 4.32 | 4.41 | 3.27 | 3.33 ± 1.05 | 0.89 |
| V942K | 2.76 | 3.24 | 4.66 | 4.73 | 4.59 | 4.02 | 4 ± 0.83 | 1.07 |
| A695R | 5.05 | 2.06 | 3.38 | 3.88 | 3.43 | 3.27 | 3.51 ± 0.97 | 0.94 |
| L363Ins | 10.77 | 6.6 | 9.33 | 11.21 | 10.99 | 11.41 | 10.05 ± 1.84 | 2.69 |
| K692Ins | 2.33 | 1.57 | 1.75 | 2.34 | 1.76 | 1.99 | 1.96 ± 0.32 | 0.52 |
| K694Ins | 6.91 | 3.36 | 6.99 | 7.68 | 6.81 | 7.18 | 6.49 ± 1.56 | 1.74 |
| WT-1 | 4.9 | 2.92 | 3.06 | 4.48 | 4.55 | 4.17 | 4.01 ± 0.83 | 1.08 |
| WT-2 | 4.68 | 1.88 | 2.83 | 4.55 | 4.46 | 4.66 | 3.84 ± 1.19 | 1.03 |
| WT-3 | 2.02 | 2.63 | 2.62 | 4.77 | 4.33 | 4.3 | 3.45 ± 1.15 | 0.92 |
| WT-4 | 3.65 | 1.9 | 2.27 | 4.51 | 3.71 | 3.29 | 3.22 ± 0.97 | 0.86 |
| WT-5 | 5.7 | 3.03 | 3.1 | 4.51 | 4.5 | 3.95 | 4.13 ± 1 | 1.11 |
| NC | 0.06 | 0.01 | 0.04 | 0.01 | 0.04 | 0.02 | 0.03 ± 0.02 | 0.01 |

| | Genomic target 2 | | | | | | | |
|---|---|---|---|---|---|---|---|---|
| | BioRep1 | | | BioRep2 | | | | Relative |
| Variants | Rep1 | Rep2 | Rep3 | Rep1 | Rep2 | Rep3 | Average | activity |
| D7A | 0.02 | 0.09 | | 0.05 | 0.06 | 0.02 | 0.05 ± 0.03 | 0 |
| I13V | 12.39 | | | 13.47 | 11.22 | 12.29 | 12.34 ± 0.92 | 0.96 |
| L19R | 16.9 | 14.72 | 15.53 | 17.42 | 14.28 | 14.55 | 15.57 ± 1.31 | 1.21 |
| T20N | 14.06 | 12.96 | 14.85 | 14.65 | 12.76 | 13.31 | 13.77 ± 0.88 | 1.07 |
| H40R | 8.44 | 7.5 | 6.26 | 8.54 | 7.38 | 6.76 | 7.48 ± 0.9 | 0.58 |
| Q69K | 13.44 | 11.62 | | 15.15 | 12.43 | 11.33 | 12.79 ± 1.55 | 0.99 |
| H70K | 18.07 | 13.81 | | 18.94 | 14.54 | 15.18 | 16.11 ± 2.26 | 1.25 |
| H70R | 14.96 | 13.4 | | 15.41 | 14.18 | 5.26 | 12.64 ± 4.2 | 0.98 |
| A71K | 19.33 | 11.18 | | 20.11 | 18.37 | 18.7 | 17.54 ± 3.62 | 1.36 |
| A71R | 4.32 | 15.14 | 18 | 20.36 | 15.87 | 16.5 | 15.03 ± 5.56 | 1.17 |
| S89C | 4.1 | 11.57 | 13.29 | 13.27 | 11.27 | 9.58 | 10.51 ± 3.44 | 0.82 |
| E105K | 12.47 | 13.86 | 19.45 | 16.45 | 15.47 | 11.9 | 14.93 ± 2.81 | 1.16 |
| T106K | 11.26 | 11.61 | 16.73 | 16.26 | 14.22 | 12.89 | 13.83 ± 2.32 | 1.07 |
| E131R | 10.96 | 14.63 | 15.19 | 15.15 | 15.23 | 12.63 | 13.97 ± 1.78 | 1.09 |
| H153K | 9.61 | 16.06 | 17.29 | 17.23 | 16.01 | 12.73 | 14.82 ± 3.05 | 1.15 |
| A180R | 16.65 | 13.32 | | 16.19 | 17.8 | 16.5 | 16.09 ± 1.66 | 1.25 |
| L185R | 11.86 | 10.54 | | 14.07 | 11.82 | 11.78 | 12.01 ± 1.28 | 0.93 |
| I215V | 4.78 | 10.85 | 15.09 | 15.13 | 13.75 | 13.32 | 12.15 ± 3.94 | 0.94 |
| N221R | 7.95 | 15.85 | 17.76 | 2.74 | 13.77 | 14.77 | 12.14 ± 5.67 | 0.94 |
| S239K | 12.17 | 14.1 | 22.49 | 22.4 | 17.08 | 15.61 | 17.31 ± 4.3 | 1.34 |
| I302V | 11.87 | 14.94 | 20.57 | 17.82 | 14.87 | 13.75 | 15.64 ± 3.1 | 1.21 |
| S366K | 13.16 | 14.42 | 14.67 | 18.06 | 14.15 | 13.76 | 14.7 ± 1.73 | 1.14 |
| W367R | 11.85 | 12.28 | 16.82 | 20.28 | 13.91 | 16.09 | 15.21 ± 3.18 | 1.18 |
| I370R | 11.09 | 10.69 | 11.21 | 12 | 11.33 | 10.6 | 11.15 ± 0.51 | 0.87 |
| D372K | 22.45 | 13.56 | | 24.59 | 20.4 | 20.9 | 20.38 ± 4.15 | 1.58 |
| A376K | 4.96 | 13.77 | 22.28 | 20.3 | 14.83 | 15.05 | 15.2 ± 6.05 | 1.18 |
| M387R | 2.38 | | 17.13 | 17.21 | 13.07 | 13.1 | 12.58 ± 6.06 | 0.98 |
| L410K | 6.57 | 13.07 | 17.13 | 15.71 | 12.89 | 11.67 | 12.84 ± 3.67 | 1 |
| T458R | 11.52 | 11.82 | 15.55 | 14.39 | 11.39 | 9.53 | 12.37 ± 2.2 | 0.96 |
| H469K | 9.25 | 13.95 | 16.82 | 16.99 | 16.16 | 12.26 | 14.24 ± 3.06 | 1.11 |
| S473K | 12.11 | 12.95 | 16.72 | 17.35 | 16.94 | 14.04 | 15.02 ± 2.27 | 1.17 |
| K495R | 13.64 | 14.04 | | 12.8 | 11.56 | 11.65 | 12.74 ± 1.13 | 0.99 |
| T537K | 15.27 | 11.75 | | 17.39 | 14.43 | 19.08 | 15.58 ± 2.81 | 1.21 |
| L538R | 3.2 | 11.18 | 17.88 | 16.51 | 11.63 | 14.46 | 12.48 ± 5.25 | 0.97 |
| H552A | 2.25 | 11.81 | 21.72 | 16.3 | 12.33 | 13.98 | 13.07 ± 6.41 | 1.02 |
| N558R | 4.52 | 12.99 | 16.46 | 15.43 | 13.72 | 15.25 | 13.06 ± 4.37 | 1.01 |
| A571K | 7.49 | 12.43 | 18.64 | 17.51 | 13.75 | 13.77 | 13.93 ± 3.97 | 1.08 |
| N577A | 0.15 | 0.15 | 0.32 | 0.47 | 0.2 | 0.32 | 0.27 ± 0.13 | 0.02 |
| H578A | 0.2 | 0.11 | 0.02 | 0.08 | 0.1 | 0.07 | 0.1 ± 0.06 | 0.01 |
| E588K | 11.41 | 10.07 | | 10.59 | 8.86 | 8.36 | 9.86 ± 1.25 | 0.77 |
| L598R | 16.59 | | | 16.52 | 15.26 | 13.47 | 15.46 ± 1.46 | 1.2 |
| S609R | 5.49 | 13.51 | 16.24 | 18.05 | 12.84 | 14.85 | 13.5 ± 4.35 | 1.05 |
| S609V | 3.96 | 12.16 | 17.47 | 17.19 | 12.73 | 13.17 | 12.78 ± 4.89 | 0.99 |
| A610R | 6.41 | 11.47 | 15.28 | 17.51 | 11.26 | 11.15 | 12.18 ± 3.84 | 0.95 |
| S611K | 5.67 | 11.89 | 16.02 | 15.55 | 12.01 | 10.67 | 11.97 ± 3.76 | 0.93 |
| A621K | 8.67 | 11.21 | 15.44 | 15.49 | 11.83 | 12.05 | 12.45 ± 2.63 | 0.97 |
| S626V | 9.16 | 11.23 | 15.2 | 16.16 | 13.39 | 10.94 | 12.68 ± 2.7 | 0.99 |
| Q657R | 12.82 | 9.81 | | 11.58 | 8.68 | 9.09 | 10.4 ± 1.75 | 0.81 |
| T688K | 18.44 | 10.57 | | 16.68 | 14.94 | 14.29 | 14.98 ± 2.95 | 1.16 |
| E689K | 3.66 | 14.05 | 19 | 20.99 | 15.9 | 16.09 | 14.95 ± 6.06 | 1.16 |
| N700K | 6.9 | 13.68 | 18.2 | 20.28 | 17.45 | 15.55 | 15.34 ± 4.71 | 1.19 |
| C736R | 4.09 | 13.39 | 21.65 | 23.55 | 18.28 | 13.77 | 15.79 ± 7.04 | 1.23 |
| N737K | 7.95 | 12.57 | 11.78 | 17.77 | 11.08 | 13.75 | 12.48 ± 3.24 | 0.97 |
| H747R | 6.09 | 10.03 | 15.95 | 15.08 | 10.72 | 12.57 | 11.74 ± 3.62 | 0.91 |
| N750R | 7.61 | 9.61 | 12.96 | 13 | 10.6 | 9.12 | 10.48 ± 2.16 | 0.81 |
| Q786R | 22.55 | 17.43 | | 20.6 | 18.15 | 15.92 | 18.93 ± 2.64 | 1.47 |
| N800R | 14.62 | 9.53 | | 15.82 | 13.15 | 15.36 | 13.7 ± 2.54 | 1.06 |
| A821R | 4.52 | 14.06 | 16.35 | 17.21 | 11.64 | 13.66 | 12.91 ± 4.57 | 1 |
| D827K | 5.36 | 14.19 | 18.31 | 16.7 | 14.21 | 14.17 | 13.82 ± 4.48 | 1.07 |
| M828R | 7.46 | 11.16 | 17.71 | 20.13 | 14.04 | 13.24 | 13.96 ± 4.53 | 1.08 |
| I843K | 21.11 | 16 | 18.65 | 27.46 | 21.24 | 19.99 | 20.74 ± 3.82 | 1.61 |
| I843R | 12.87 | 18.75 | 22.15 | 22.18 | 21.18 | 17.47 | 19.1 ± 3.6 | 1.48 |
| L847K | 12.28 | 13.3 | 19.67 | 18.73 | 16.37 | 14.18 | 15.76 ± 3 | 1.22 |
| A848R | 21.48 | 13.65 | | 19.52 | 16.04 | 14.33 | 17 ± 3.38 | 1.32 |
| L853K | 19.63 | 14.9 | | 20.81 | 17.46 | 18.19 | 18.2 ± 2.25 | 1.41 |
| H866K | 4.24 | 12.18 | 17.48 | 16.72 | 11.68 | 14.28 | 12.76 ± 4.78 | 0.99 |
| L873W | 9.41 | 12.45 | 14.83 | 15.28 | 12.74 | 11.57 | 12.71 ± 2.16 | 0.99 |
| A901E | 6.42 | 10.45 | | 17.51 | 15.7 | 15.11 | 13.04 ± 4.52 | 1.01 |
| A901K | 14.62 | 11.65 | 13.98 | 17.27 | 12.46 | 11.52 | 13.58 ± 2.19 | 1.06 |
| V908I | 11.98 | 11.83 | 16.91 | 16.37 | 14.09 | 11.55 | 13.79 ± 2.39 | 1.07 |
| R909A | 2.69 | 3.45 | 2.29 | 3.87 | 2.51 | 2.31 | 2.85 ± 0.66 | 0.22 |
| N910A | 4.68 | 3.41 | | 3.86 | 3.11 | 3.36 | 3.68 ± 0.62 | 0.29 |
| F911A | 3.95 | 1.74 | | 3.93 | 3.14 | 2.77 | 3.11 ± 0.92 | 0.24 |
| D912S | 2 | 6.35 | 6.9 | 8.36 | 6.42 | 6.52 | 6.09 ± 2.14 | 0.47 |
| Q913K | 8.39 | 19.23 | 24.26 | 9.98 | 20.94 | 21.25 | 17.34 ± 6.54 | 1.35 |
| Q913S | 9.2 | 11.9 | 19.7 | 18.28 | 14.85 | 13.59 | 14.59 ± 3.93 | 1.13 |
| S914N | 8.92 | 7.03 | 11.47 | 12.04 | 11.75 | 8.12 | 9.89 ± 2.14 | 0.77 |
| K915Y | 5.06 | 6.19 | 6.03 | 7.23 | 5.08 | 6 | 5.93 ± 0.81 | 0.46 |
| D916G | 11.75 | 12.64 | 13.08 | 18.44 | 13.48 | 13.58 | 13.83 ± 2.36 | 1.07 |
| K918R | 13.47 | 11 | | 12.19 | 10.21 | 8.91 | 11.16 ± 1.76 | 0.87 |
| F920K | 22.48 | 7.15 | | 18.31 | 14.23 | 14.77 | 15.39 ± 5.67 | 1.2 |
| F920S | 5.01 | 10.59 | 15.71 | 16.08 | 12.79 | 12.63 | 12.14 ± 4.05 | 0.94 |
| R921I | 4.95 | 4.32 | 8.68 | 7.39 | 4.99 | 6.48 | 6.14 ± 1.68 | 0.48 |
| I928V | 12.37 | 10.62 | 15.5 | 17.59 | 11.85 | 12.45 | 13.4 ± 2.61 | 1.04 |
| K930R | 8.54 | 6.49 | 13.45 | 14.84 | 10.22 | 9.99 | 10.59 ± 3.09 | 0.82 |
| V942K | 12.37 | 11.98 | 16.24 | 15.66 | 13.05 | 12.65 | 13.66 ± 1.82 | 1.06 |
| A695R | 7.19 | 5.85 | 10.91 | 12.97 | 11.15 | 9.93 | 9.67 ± 2.66 | 0.75 |
| L363Ins | 9.11 | 7.16 | | 9.39 | 9.54 | 7.58 | 8.56 ± 1.1 | 0.66 |
| K692Ins | 7.23 | | | 9.58 | 8.74 | 8.73 | 8.57 ± 0.98 | 0.67 |
| K694Ins | 19.11 | 14.36 | 13.88 | 25.51 | 21.69 | 21.93 | 19.41 ± 4.58 | 1.51 |
| WT-1 | 15.14 | | 6.42 | 16.44 | 10.82 | 13.48 | 12.46 ± 3.98 | 0.97 |
| WT-2 | 15.18 | 6.11 | | 16.08 | 16.52 | 13.06 | 13.39 ± 4.28 | 1.04 |
| WT-3 | 10.54 | 3.87 | 7.52 | 15.98 | 14.55 | 13.73 | 11.03 ± 4.65 | 0.86 |
| WT-4 | 13.47 | 10.64 | | 15.92 | 14.89 | 14.17 | 13.82 ± 1.99 | 1.07 |
| WT-5 | 14.68 | 9.86 | 12.31 | 16.91 | 13.38 | 15.83 | 13.83 ± 2.55 | 1.07 |
| NC | 0.01 | 0.06 | 0.05 | 0.05 | 0.05 | 0.02 | 0.04 ± 0.02 | 0 |

### Example 3: Variable Spacer Truncation for Cas9-IID sgRNA

To define the optimal length of the variable region, the on-target activity of Cas9-IID variants was first measured in human cells using WT guideRNA with 18-25 nt variable region over 2 target sites each. Nuclease and guide expression plasmids were co-transfected into HEK293-FT cells using LIPOFECTAMINE 2000 under recommended condition. Gene editing activity was measured by amplicon sequencing 3-days post transfection.

TABLE 3A
Truncated variable region of sgRNA. To facilitate efficient RNA transcription under human
U6 promoter, additional G is appended to the 5′-end if the initial nucleotide is not a G. This
can cause duplication during truncation whenever there is a G in the variable region, which are
bolded in the table. SI# refers to the SEQ ID NO of the sequence to the number's immediate left.

| | | | | | |
|---|---|---|---|---|---|
| | Length of | | | | |
| Genomic | variable | | | Sequence with additional G | |
| Target | region | Sequence | SI# | if applicable | SI# |

Cas9-IID25 (SEQ ID NO: 1)

| | | | | | |
|---|---|---|---|---|---|
| VEGFA1 | 25 | tcaaccccacacgcacacactcact | 123 | Gtcaaccccacacgcacacactcact | 139 |
| | 24 | caaccccacacgcacacactcact | 124 | Gcaaccccacacgcacacactcact | 140 |
| | 23 | aaccccacacgcacacactcact | 125 | Gaaccccacacgcacacactcact | 141 |
| | 22 | accccacacgcacacactcact | 126 | Gaccccacacgcacacactcact | 142 |
| | 21 | ccccacacgcacacactcact | 127 | Gccccacacgcacacactcact | 143 |
| | 20 | cccacacgcacacactcact | 128 | Gcccacacgcacacactcact | 144 |
| | 19 | ccacacgcacacactcact | 129 | Gccacacgcacacactcact | 145 |
| | 18 | cacacgcacacactcact | 130 | Gcacacgcacacactcact | 146 |
| HPRT3807 | 25 | actacagacaaggcacttggttgat | 131 | Gactacagacaaggcacttggttgat | 147 |
| | 24 | ctacagacaaggcacttggttgat | 132 | Gctacagacaaggcacttggttgat | 148 |
| | 23 | tacagacaaggcacttggttgat | 133 | Gtacagacaaggcacttggttgat | 149 |
| | 22 | acagacaaggcacttggttgat | 134 | Gacagacaaggcacttggttgat | 150 |
| | 21 | cagacaaggcacttggttgat | 135 | Gcagacaaggcacttggttgat | 151 |
| | 20 | agacaaggcacttggttgat | 136 | Gagacaaggcacttggttgat | 152 |
| | 19 | gacaaggcacttggttgat | 137 | gacaaggcacttggttgat | 153 |
| | 18 | acaaggcacttggttgat | 138 | Gacaaggcacttggttgat | 154 |

Cas9-IID49 (SEQ ID NO: 5)

| | | | | | |
|---|---|---|---|---|---|
| EMX12 | 25 | gtcacctccaatgactagggtgggc | 155 | gtcacctccaatgactagggtgggc | 171 |
| | 24 | tcacctccaatgactagggtgggc | 156 | Gtcacctccaatgactagggtgggc | 172 |
| | 23 | cacctccaatgactagggtgggc | 157 | Gcacctccaatgactagggtgggc | 173 |
| | 22 | acctccaatgactagggtgggc | 158 | Gacctccaatgactagggtgggc | 174 |
| | 21 | cctccaatgactagggtgggc | 159 | Gcctccaatgactagggtgggc | 175 |
| | 20 | ctccaatgactagggtgggc | 160 | Gctccaatgactagggtgggc | 176 |
| | 19 | tccaatgactagggtgggc | 161 | Gtccaatgactagggtgggc | 177 |
| | 18 | ccaatgactagggtgggc | 162 | Gccaatgactagggtgggc | 178 |
| HPRT38285 | 25 | ttcacttacagtctggcttatatcc | 163 | Gttcacttacagtctggcttatatcc | 179 |
| | 24 | tcacttacagtctggcttatatcc | 164 | Gtcacttacagtctggcttatatcc | 180 |
| | 23 | cacttacagtctggcttatatcc | 165 | Gcacttacagtctggcttatatcc | 181 |
| | 22 | acttacagtctggcttatatcc | 166 | Gacttacagtctggcttatatcc | 182 |
| | 21 | cttacagtctggcttatatcc | 167 | Gcttacagtctggcttatatcc | 183 |
| | 20 | ttacagtctggcttatatcc | 168 | Gttacagtctggcttatatcc | 184 |
| | 19 | tacagtctggcttatatcc | 169 | Gtacagtctggcttatatcc | 185 |
| | 18 | acagtctggcttatatcc | 170 | Gacagtctggcttatatcc | 186 |

Cas9-IID59 (SEQ ID NO: 7)

| | | | | | |
|---|---|---|---|---|---|
| VEGFA1 | 25 | gccttctccccgctccaacgccctc | 187 | gccttctccccgctccaacgccctc | 203 |
| | 24 | ccttctccccgctccaacgccctc | 188 | Gccttctccccgctccaacgccctc | 204 |
| | 23 | cttctccccgctccaacgccctc | 189 | Gcttctccccgctccaacgccctc | 205 |
| | 22 | ttctccccgctccaacgccctc | 190 | Gttctccccgctccaacgccctc | 206 |
| | 21 | tctccccgctccaacgccctc | 191 | Gtctccccgctccaacgccctc | 207 |
| | 20 | ctccccgctccaacgccctc | 192 | Gctccccgotccaacgccctc | 208 |
| | 19 | tccccgctccaacgccctc | 193 | Gtccccgctccaacgccctc | 209 |
| | 18 | ccccgctccaacgccctc | 194 | Gccccgctccaacgccctc | 210 |
| HPRT38285 | 25 | ttcacttacagtctggettatatcc | 195 | Gttcacttacagtctggcttatatcc | 211 |
| | 24 | tcacttacagtctggcttatatcc | 196 | Gtcacttacagtctggcttatatcc | 212 |
| | 23 | cacttacagtctggcttatatcc | 197 | Gcacttacagtctggcttatatcc | 213 |
| | 22 | acttacagtctggcttatatcc | 198 | Gacttacagtctggcttatatcc | 214 |
| | 21 | cttacagtctggcttatatcc | 199 | Gcttacagtctggcttatatcc | 215 |
| | 20 | ttacagtctggcttatatcc | 200 | Gttacagtctggcttatatcc | 216 |
| | 19 | tacagtctggcttatatcc | 201 | Gtacagtctggcttatatcc | 217 |
| | 18 | acagtctggcttatatcc | 202 | Gacagtctggcttatatcc | 218 |

Cas9-IID64 (SEQ ID NO: 9)

| | | | | | |
|---|---|---|---|---|---|
| EMX11 | 25 | ggcctgagtccgagcagaagaagaa | 219 | ggcctgagtccgagcagaagaagaa | 235 |
| | 24 | gcctgagtccgagcagaagaagaa | 220 | gcctgagtccgagcagaagaagaa | 236 |
| | 23 | cctgagtccgagcagaagaagaa | 221 | Gcctgagtccgagcagaagaagaa | 237 |
| | 22 | ctgagtccgagcagaagaagaa | 222 | Gctgagtccgagcagaagaagaa | 238 |
| | 21 | tgagtccgagcagaagaagaa | 223 | Gtgagtccgagcagaagaagaa | 239 |
| | 20 | gagtccgagcagaagaagaa | 224 | gagtccgagcagaagaagaa | 240 |
| | 19 | agtccgagcagaagaagaa | 225 | Gagtccgagcagaagaagaa | 241 |
| | 18 | gtccgagcagaagaagaa | 226 | gtccgagcagaagaagaa | 242 |
| EMX13 | 25 | agggtgggcaaccacaaacccacga | 227 | Gagggtgggcaaccacaaacccacga | 243 |
| | 24 | gggtgggcaaccacaaacccacga | 228 | gggtgggcaaccacaaacccacga | 244 |
| | 23 | ggtgggcaaccacaaacccacga | 229 | ggtgggcaaccacaaacccacga | 245 |
| | 22 | gtgggcaaccacaaacccacga | 230 | gtgggcaaccacaaacccacga | 246 |
| | 21 | tgggcaaccacaaacccacga | 231 | Gtgggcaaccacaaacccacga | 247 |
| | 20 | gggcaaccacaaacccacga | 232 | gggcaaccacaaacccacga | 248 |
| | 19 | ggcaaccacaaacccacga | 233 | ggcaaccacaaacccacga | 249 |
| | 18 | gcaaccacaaacccacga | 234 | gcaaccacaaacccacga | 250 |

Cas9-IID65 (SEQ ID NO: 10)

| | | | | | |
|---|---|---|---|---|---|
| VEGFA1 | 25 | ggggttgagggcgttggagcgggga | 251 | ggggttgagggcgttggagcgggga | 267 |
| | 24 | gggttgagggcgttggagcgggga | 252 | gggttgagggcgttggagcgggga | 268 |
| | 23 | ggttgagggcgttggagcgggga | 253 | ggttgagggcgttggagcgggga | 269 |
| | 22 | gttgagggcgttggagcgggga | 254 | gttgagggcgttggagcgggga | 270 |
| | 21 | ttgagggcgttggagcgggga | 255 | Gttgagggcgttggagcgggga | 271 |
| | 20 | tgagggcgttggagcgggga | 256 | Gtgagggcgttggagcgggga | 272 |
| | 19 | gagggcgttggagcgggga | 257 | gagggcgttggagcgggga | 273 |
| | 18 | agggcgttggagcgggga | 258 | Gagggcgttggagcgggga | 274 |
| EMX13 | 25 | tccaatgactagggtgggcaaccac | 259 | Gtccaatgactagggtgggcaaccac | 275 |
| | 24 | ccaatgactagggtgggcaaccac | 260 | Gccaatgactagggtgggcaaccac | 276 |
| | 23 | caatgactagggtgggcaaccac | 261 | Gcaatgactagggtgggcaaccac | 277 |
| | 22 | aatgactagggtgggcaaccac | 262 | Gaatgactagggtgggcaaccac | 278 |
| | 21 | atgactagggtgggcaaccac | 263 | Gatgactagggtgggcaaccac | 279 |
| | 20 | tgactagggtgggcaaccac | 264 | Gtgactagggtgggcaaccac | 280 |
| | 19 | gactagggtgggcaaccac | 265 | gactagggtgggcaaccac | 281 |
| | 18 | actaggggggcaaccac | 266 | Gactagggtgggcaaccac | 282 |

Cas9-IID82 (SEQ ID NO: 11)

| | | | | | |
|---|---|---|---|---|---|
| EMX12 | 25 | gtcacctccaatgactagggtgggc | 283 | gtcacctccaatgactagggtgggc | 299 |
| | 24 | tcacctccaatgactagggtgggc | 284 | Gtcacctccaatgactagggtgggc | 300 |
| | 23 | cacctccaatgactagggtgggc | 285 | Gcacctccaatgactagggtgggc | 301 |
| | 22 | acctccaatgactagggtgggc | 286 | Gacctccaatgactagggtgggc | 302 |
| | 21 | cctccaatgactagggtgggc | 287 | Gcctccaatgactagggtgggc | 303 |
| | 20 | ctccaatgactagggtgggc | 288 | Gctccaatgactagggtgggc | 304 |
| | 19 | tccaatgactagggtgggc | 289 | Gtccaatgactagggtgggc | 305 |
| | 18 | ccaatgactagggtgggc | 290 | Gccaatgactagggtgggc | 306 |
| VEGFA1 | 25 | aaccccacacgcacacactcactca | 291 | Gaaccccacacgcacacactcactca | 307 |
| | 24 | accccacacgcacacactcactca | 292 | Gaccccacacgcacacactcactca | 308 |
| | 23 | ccccacacgcacacactcactca | 293 | Gccccacacgcacacactcactca | 309 |
| | 22 | cccacacgcacacactcactca | 294 | Gcccacacgcacacactcactca | 310 |
| | 21 | ccacacgcacacactcactca | 295 | Gccacacgcacacactcactca | 311 |
| | 20 | cacacgcacacactcactca | 296 | Gcacacgcacacactcactca | 312 |
| | 19 | acacgcacacactcactca | 297 | Gacacgcacacactcactca | 313 |
| | 18 | cacgcacacactcactca | 298 | Gcacgcacacactcactca | 314 |

Tables 3B-3M show the gene editing activity of Cas9-IID variants in human HEK293-FT cells using truncated variable sequences in the sgRNA from Table 3A.

TABLE 3B
Cas9-IID25
Cas9-IID25 Target 1 (VEGFA1) Editing efficiency (%)

| Length of variable region | Replicate 1 | Replicate 2 | Replicate 3 |
|---|---|---|---|
| 25 | 12.68 | 14.02 | 12.43 |
| 24 | 29.78 | 30.33 | 28.23 |
| 23 | 18.11 | 23.8 | 19.33 |
| 22 | 22.27 | 24.11 | 28.2 |
| 21 | 38.77 | 46.05 | 40.84 |
| 20 | 36.29 | 39.92 | 32.16 |
| 19 | 28.97 | 32.53 | 25.26 |
| 18 | 2.26 | 3.95 | 3.7 |

TABLE 3C
Cas9-IID25 Target 2 (HPRT3807) Editing efficiency(%)

| Length of variable region | Replicate 1 | Replicate 2 | Replicate 3 |
|---|---|---|---|
| 25 | 1.66 | 1.98 | 1.66 |
| 24 | 4.14 | 4.95 | 4.34 |
| 23 | 4.81 | 5.58 | 5.2 |
| 22 | 6.97 | 9.73 | 7.68 |
| 21 | 20.39 | 26.33 | 21.47 |
| 20 | 20.17 | 25.47 | 22.39 |
| 18/19 | 1.72 | 2.19 | 2.01 |

TABLE 3D
Cas9-IID49
Cas9-IID49 Target 1 (EMX12) Editing efficiency (%)

| Length of variable region | Replicate 1 | Replicate 2 | Replicate 3 |
|---|---|---|---|
| 24/25 | 1.56 | 3.01 | 3.26 |
| 23 | 4.43 | 5.99 | 6.32 |
| 22 | 6.5 | 7.05 | 8.52 |
| 21 | 13 | 17.29 | 13.82 |
| 20 | 8.43 | 10.82 | 9.28 |
| 19 | 0.75 | 1.4 | 1.06 |
| 18 | 0.14 | 0.16 | 0.03 |

TABLE 3E
Cas9-IID49 Target 2 (HPRT38285) Editing efficiency (%)

| Length of variable region | Replicate 1 | Replicate 2 | Replicate 3 |
|---|---|---|---|
| 25 | 0.07 | 0.09 | 0.04 |
| 24 | 0.11 | 0.12 | 0.09 |
| 23 | 0.09 | 0.11 | 0.12 |
| 22 | 0.09 | 0.02 | 0.14 |
| 21 | 0.04 | 0.01 | 0.01 |
| 20 | 0.02 | 0.02 | 0.01 |
| 19 | 0.01 | 0.01 | 0.01 |
| 18 | 0.01 | 0.02 | 0.01 |

TABLE 3F
Cas9-IID59
Cas9-IID59 Target 1 (VEGFA1) Editing efficiency (%)

| Length of variable region | Replicate 1 | Replicate 2 | Replicate 3 |
|---|---|---|---|
| 24/25 | 0.17 | 0.15 | 0.09 |
| 23 | 0.17 | 0.21 | 0.18 |
| 22 | 0.42 | 0.53 | 0.68 |
| 21 | 0.9 | 0.95 | 1.16 |
| 20 | 0.55 | 0.43 | 0.45 |
| 19 | 0.13 | 0.8 | 0.43 |
| 18 | 0.04 | 0.02 | 0.02 |

TABLE 3G
Cas9-IID59 Target 2 Editing efficiency (%)

| Length of variable region | Replicate 1 | Replicate 2 | Replicate 3 |
|---|---|---|---|
| 25 | 0.25 | 0.46 | 0.44 |
| 24 | 0.71 | 1.34 | 1.24 |
| 23 | 1.25 | 2.67 | 2.54 |
| 22 | 2.25 | 3.36 | 3.3 |
| 21 | 3.75 | 4.29 | 4.03 |
| 20 | 4.34 | 6.22 | 5.28 |
| 19 | 2.47 | 3.84 | 2.44 |
| 18 | 0.09 | 0.34 | 0.2 |

TABLE 3H
Cas9-IID64
Cas9-IID64 Target 1 (EMX11) Editing efficiency (%)

| Length of variable region | Replicate 1 | Replicate 2 | Replicate 3 |
|---|---|---|---|
| 25 | 0.03 | 0.05 | 0.03 |
| 23/24 | 0.15 | 0.15 | 0.1 |
| 22 | 0.01 | 0.02 | 0.02 |
| 21 | 0.01 | 0.02 | 0.02 |
| 19/20 | 0.01 | 0.02 | 0.02 |
| 18 | 0.02 | 0.02 | 0.03 |

TABLE 3I
Cas9-IID64 Target 2 (EMX13) Editing efficiency (%)

| Length of variable region | Replicate 1 | Replicate 2 | Replicate 3 |
|---|---|---|---|
| 25 | 0.02 | 0.02 | 0.02 |
| 24 | 0.02 | 0.03 | 0.03 |
| 23 | 0.06 | 0.03 | 0.01 |
| 21/22 | 0.02 | 0.03 | 0.02 |
| 20 | 0.01 | 0.02 | 0.02 |
| 19 | 0.02 | 0.02 | 0.02 |
| 18 | 0.01 | 0.02 | 0.02 |

TABLE 3J
Cas9-IID65
Cas9-IID65 Target 1 (VEGFA1) Editing efficiency (%)

| Length of variable region | Replicate 1 | Replicate 2 | Replicate 3 |
|---|---|---|---|
| 25 | 0.17 | 0.15 | 0.32 |
| 24 | 0.25 | 0.35 | 0.53 |
| 23 | 0.38 | 0.36 | 0.5 |
| 21/22 | 0.36 | 0.34 | 0.38 |
| 20 | 0.55 | 0.74 | 0.61 |
| 18/19 | 0.21 | 0.39 | 0.19 |

TABLE 3K
Cas9-IID65 Target 2 (EMX13) Editing efficiency (%)

| Length of variable region | Replicate 1 | Replicate 2 | Replicate 3 |
|---|---|---|---|
| 25 | 0.01 | 0 | 0.01 |
| 24 | 0.01 | 0.01 | 0.01 |
| 23 | 0.01 | 0 | 0.02 |
| 22 | 0.01 | 0.03 | 0.03 |
| 21 | 0.14 | 0.13 | 0.09 |
| 20 | 0.15 | 0.11 | 0.06 |
| 18/19 | 0.06 | 0.02 | 0.05 |

TABLE 3L
Cas9-IID82
Cas9-IID82 Target 1 (EMX12) Editing efficiency (%)

| Length of variable region | Replicate 1 | Replicate 2 | Replicate 3 |
|---|---|---|---|
| 24/25 | 0.72 | 1.26 | 0.97 |
| 23 | 1.88 | 2.32 | 2.11 |
| 22 | 3.7 | 4.31 | 3.93 |
| 21 | 6.98 | 8.7 | 6.85 |
| 20 | 5.95 | 6.16 | 5.85 |
| 19 | 0.94 | 1.37 | 1.17 |
| 18 | 0.04 | 0.07 | 0.03 |

TABLE 3M
Cas9-IID82 Target 2 (VEGFA1) Editing efficiency (%)

| Length of variable region | Replicate 1 | Replicate 2 | Replicate 3 |
|---|---|---|---|
| 25 | 0.29 | 0.11 | 0.03 |
| 24 | 0.11 | 0.08 | 0.04 |
| 23 | 0.12 | 0.12 | 0.04 |
| 22 | 0.1 | 0.07 | 0.03 |
| 21 | 0.22 | 0.25 | 0.05 |
| 20 | 0.08 | 0.11 | 0.17 |
| 19 | 0.19 | 0.04 | 0 |
| 18 | 0.15 | 0.11 | 0.12 |

### Example 4: Cas9-IID sgRNA Trimming

Cas9-IID-25 is a type-II CRISPR system that employs a 178-nt sgRNA for RNA-guided DNA targeting. The extended length of sgRNA poses manufacturing challenges, thereby hindering the translational application of this gene editing system. The predicted structural regions of the IID-25 sgRNA were systematically perturbed, and a set of truncated variants was discovered that exhibited comparable or even greater activity than the wild-type (WT) counterpart. By combining these truncated variants, the sgRNA length can be reduced from 178-nt to 138-nt, which can substantially enhance the manufacturability of sgRNA for clinical applications.

To initiate the RNA engineering process, the Vienna RNA folding algorithm was first utilized to predict the secondary structure of the wild-type sgRNA (scaffold-only). Seven structural elements, comprising stem loop 1 to 6 (S1-6) and 3′-end regions, were defined manually (see e.g., Structure A in FIG. 3, e.g., SEQ ID NO: 409).

Next, a panel of 94 scaffold variants was designed with the goal of reducing the length of sgRNA, or modifying the sequence composition to augment the nuclease activity (see e.g., Table 4). Plasmids encoding each sgRNA variant were co-transfected with the nuclease plasmid to HEK293-FT cells by LIPOFECTAMINE 2000. Genomic DNA was then extracted 72 hours after transfection using the QUICKEXTRACTION solution (LUCIGEN). The specific genomic region targeted by the 5′-end 21-nt spacer sequence of the sgRNA was subsequently amplified, purified, and subjected to deep sequencing using an ILLUMINA MISEQ instrument. The editing efficiency of IID-25 (SEQ ID NO: 1) with each sgRNA variant was determined by percentage of indel formation.

TABLE 4
sgRNA Modifications, SI# refers to the SEQ ID NO of the sequence to the
number's immediate left.

| | Modified | | | | | |
|---|---|---|---|---|---|---|
| Variants | Region | Action | Scaffold sequence | SI# | sgRNA (EMX1-3) | SI# |
| V1 | S1 | Del 1- | guuucaaucaaccgaugaaaaucg | 315 | GCUCGUGGGUUUGUG | 411 |
| | | bp | guccgguugaaaagagcaucgguc | | GUUGCCguuucaaucaaccg | |
| | | | ugaaggaugcacuccgggauaggg | | augaaaaucgguccgguugaaaa | |
| | | | cagucccggcucuugcuguuuccc | | gagcaucggucugaaggaugca | |
| | | | cgguaagaccucggaagcaagucc | | cuccgggauagggcagucccgg | |
| | | | uucagcaagucgaaagacacgaug | | cucuugcuguuuccccgguaag | |
| | | | ugagccuauuuu | | accucggaagcaaguccuucagc | |
| | | | | | aagucgaaagacacgaugugagc | |
| | | | | | cuauuuu | |
| V2 | S1 | Del 2- | guuucaaucaaccgagaaaucggu | 316 | GCUCGUGGGUUUGUG | 412 |
| | | bp | ccgguugaaaagagcaucggucug | | GUUGCCguuucaaucaaccg | |
| | | | aaggaugcacuccgggauagggca | | agaaaucgguccgguugaaaaga | |
| | | | gucccggcucuugcuguuuccccg | | gcaucggucugaaggaugcacu | |
| | | | guaagaccucggaagcaaguccuu | | ccgggauagggcagucccggcu | |
| | | | cagcaagucgaaagacacgauguga | | cuugcuguuuccccgguaagac | |
| | | | gccuauuuu | | cucggaagcaaguccuucagcaa | |
| | | | | | gucgaaagacacgaugugagccu | |
| | | | | | auuuu | |
| V3 | S1 | Del 3- | guuucaaucaaccggaaacggucc | 317 | GCUCGUGGGUUUGUG | 413 |
| | | bp | gguugaaaagagcaucggucugaa | | GUUGCCguuucaaucaaccg | |
| | | | ggaugcacuccgggauagggcagu | | gaaacgguccgguugaaaagagc | |
| | | | cccggcucuugcuguuuccccggu | | aucggucugaaggaugcacucc | |
| | | | aagaccucggaagcaaguccuucag | | gggauagggcagucccggcucu | |
| | | | caagucgaaagacacgaugugagcc | | ugcuguuuccccgguaagaccu | |
| | | | uauuuu | | cggaagcaaguccuucagcaagu | |
| | | | | | cgaaagacacgaugugagccuau | |
| | | | | | uuu | |
| V4 | S1 | Del 4- | guuucaaucaaccgaaagguccgg | 318 | GCUCGUGGGUUUGUG | 414 |
| | | bp | uugaaaagagcaucggucugaagg | | GUUGCCguuucaaucaaccg | |
| | | | augcacuccgggauagggcagucc | | aaagguccgguugaaaagagcau | |
| | | | cggcucuugcuguuuccccgguaa | | cggucugaaggaugcacuccgg | |
| | | | gaccucggaagcaaguccuucagca | | gauagggcagucccggcucuug | |
| | | | agucgaaagacacgaugugagccu | | cuguuuccccgguaagaccucg | |
| | | | auuuu | | gaagcaaguccuucagcaagucg | |
| | | | | | aaagacacgaugugagccuauuuu | |
| V5 | S1 | Del 5- | guuucaaucaacgaaaguccgguu | 319 | GCUCGUGGGUUUGUG | 415 |
| | | bp | gaaaagagcaucggucugaaggau | | GUUGCCguuucaaucaacga | |
| | | | gcacuccgggauagggcagucccg | | aaguccgguugaaaagagcaucg | |
| | | | gcucuugcuguuuccccgguaaga | | gucugaaggaugcacuccggga | |
| | | | ccucggaagcaaguccuucagcaag | | uagggcagucccggcucuugcu | |
| | | | ucgaaagacacgaugugagccuau | | guuuccccgguaagaccucggaa | |
| | | | uuu | | gcaaguccuucagcaagucgaaa | |
| | | | | | gacacgaugugagccuauuuu | |
| V6 | S1 | Del 6- | guuucaaucaagaaauccgguuga | 320 | GCUCGUGGGUUUGUG | 416 |
| | | bp | aaagagcaucggucugaaggaugc | | GUUGCCguuucaaucaagaa | |
| | | | acuccgggauagggcagucccggc | | auccgguugaaaagagcaucgg | |
| | | | ucuugcuguuuccccgguaagacc | | ucugaaggaugcacuccgggau | |
| | | | ucggaagcaaguccuucagcaagu | | agggcagucccggcucuugcug | |
| | | | cgaaagacacgaugugagccuauu | | uuuccccgguaagaccucggaag | |
| | | | uu | | caaguccuucagcaagucgaaag | |
| | | | | | acacgaugugagccuauuuu | |
| V7 | S1 | Del 6- | guuucaaucaauccgguugaaaag | 321 | GCUCGUGGGUUUGUG | 417 |
| | | bp, no | agcaucggucugaaggaugcacuc | | GUUGCCguuucaaucaaucc | |
| | | GAAA | cgggauagggcagucccggcucuu | | gguugaaaagagcaucggucug | |
| | | | gcuguuuccccgguaagaccucgg | | aaggaugcacuccgggauaggg | |
| | | | aagcaaguccuucagcaagucgaaa | | cagucccggcucuugcuguuuc | |
| | | | gacacgaugugagccuauuuu | | cccgguaagaccucggaagcaag | |
| | | | | | uccuucagcaagucgaaagacac | |
| | | | | | gaugugagccuauuuu | |
| V8 | S1 | Del 8- | guuucaaucgaaagguugaaaaga | 322 | GCUCGUGGGUUUGUG | 418 |
| | | bp | gcaucggucugaaggaugcacucc | | GUUGCCguuucaaucgaaag | |
| | | | gggauagggcagucccggcucuug | | guugaaaagagcaucggucuga | |
| | | | cuguuuccccgguaagaccucgga | | aggaugcacuccgggauagggc | |
| | | | agcaaguccuucagcaagucgaaag | | agucccggcucuugcuguuucc | |
| | | | acacgaugugagccuauuuu | | ccgguaagaccucggaagcaagu | |
| | | | | | ccuucagcaagucgaaagacacg | |
| | | | | | augugagccuauuuu | |
| V9 | S1 | Del 9- | guuucaaugaaaguugaaaagagc | 323 | GCUCGUGGGUUUGUG | 419 |
| | | bp | aucggucugaaggaugcacuccgg | | GUUGCCguuucaaugaaag | |
| | | | gauagggcagucccggcucuugcu | | uugaaaagagcaucggucugaa | |
| | | | guuuccccgguaagaccucggaag | | ggaugcacuccgggauagggca | |
| | | | caaguccuucagcaagucgaaagac | | gucccggcucuugcuguuuccc | |
| | | | acgaugugagccuauuuu | | cgguaagaccucggaagcaaguc | |
| | | | | | cuucagcaagucgaaagacacga | |
| | | | | | ugugagccuauuuu | |
| V10 | S1 | Del 10- | guuucaagaaauugaaaagagcau | 324 | GCUCGUGGGUUUGUG | 420 |
| | | bp | cggucugaaggaugcacuccggga | | GUUGCCguuucaagaaauu | |
| | | | uagggcagucccggcucuugcugu | | gaaaagagcaucggucugaagga | |
| | | | uuccccgguaagaccucggaagcaa | | ugcacuccgggauagggcaguc | |
| | | | guccuucagcaagucgaaagacacg | | ccggcucuugcuguuuccccgg | |
| | | | augugagccuauuuu | | uaagaccucggaagcaaguccuu | |
| | | | | | cagcaagucgaaagacacgaugu | |
| | | | | | gagccuauuuu | |
| V11 | S1 | Del 11- | guuucagaaaugaaaagagcaucg | 325 | GCUCGUGGGUUUGUG | 421 |
| | | bp | gucugaaggaugcacuccgggaua | | GUUGCCguuucagaaaugaa | |
| | | | gggcagucccggcucuugcuguuu | | aagagcaucggucugaaggaug | |
| | | | ccccgguaagaccucggaagcaagu | | cacuccgggauagggcagucccg | |
| | | | ccuucagcaagucgaaagacacgau | | gcucuugcuguuuccccgguaa | |
| | | | gugagccuauuuu | | gaccucggaagcaaguccuucag | |
| | | | | | caagucgaaagacacgaugugag | |
| | | | | | ccuauuuu | |
| V12 | S1 | Alt V1 | guuucaaaaccgauagaaauaucg | 326 | GCUCGUGGGUUUGUG | 422 |
| | | | guccuugaaaagagcaucggucug | | GUUGCCguuucaaaaccgau | |
| | | | aaggaugcacuccgggauagggca | | agaaauaucgguccuugaaaaga | |
| | | | gucccggcucuuhcuguuuccccg | | gcaucggucugaaggaugcacu | |
| | | | guaagaccucggaagcaaguccuu | | ccgggauagggcagucccggcu | |
| | | | cagcaagucgaaagacacgauguga | | cuugcuguuuccccgguaagac | |
| | | | gccuauuuu | | cucggaagcaaguccuucagcaa | |
| | | | | | gucgaaagacacgaugugagccu | |
| | | | | | auuuu | |
| V13 | S1 | Alt V2 | guuucaaaccgauagaaauaucgg | 327 | GCUCGUGGGUUUGUG | 423 |
| | | | uccuugaaaagagcaucggucuga | | GUUGCCguuucaaaccgaua | |
| | | | aggaugcacuccgggauagggcag | | gaaauaucgguccuugaaaagag | |
| | | | ucccggcucuugcuguuuccccgg | | caucggucugaaggaugcacucc | |
| | | | uaagaccucggaagcaaguccuuca | | gggauagggcagucccggcucu | |
| | | | gcaagucgaaagacacgaugugag | | ugcuguuuccccgguaagaccu | |
| | | | ccuauuuu | | cggaagcaaguccuucagcaagu | |
| | | | | | cgaaagacacgaugugagccuau | |
| | | | | | uuu | |
| V14 | S1 | Alt V3 | guuucaaaccgauagaaauaucgg | 328 | GCUCGUGGGUUUGUG | 424 |
| | | | uccgguugaaaagagcaucggucu | | GUUGCCguuucaaaccgaua | |
| | | | gaaggaugcacuccgggauagggc | | gaaauaucgguccgguugaaaa | |
| | | | agucccggcucuugcuguuucccc | | gagcaucggucugaaggaugca | |
| | | | gguaagaccucggaagcaaguccu | | cuccgggauagggcagucccgg | |
| | | | ucagcaagucgaaagacacgaugu | | cucuugcuguuuccccgguaag | |
| | | | gagccuauuuu | | accucggaagcaaguccuucagc | |
| | | | | | aagucgaaagacacgaugugagc | |
| | | | | | cuauuuu | |
| V15 | S1 | Alt V4 | guuucaaaccgauagaaauaucgg | 329 | GCUCGUGGGUUUGUG | 425 |
| | | | uccUGAuugaaaagagcaucggu | | GUUGCCguuucaaaccgaua | |
| | | | cugaaggaugcacuccgggauagg | | gaaauaucgguccUGAuugaa | |
| | | | gcagucccggcucuugcuguuucc | | aagagcaucggucugaaggaug | |
| | | | ccgguaagaccucggaagcaagucc | | cacuccgggauagggcagucccg | |
| | | | uucagcaagucgaaagacacgaug | | gcucuugcuguuuccccgguaa | |
| | | | ugagccuauuuu | | gaccucggaagcaaguccuucag | |
| | | | | | caagucgaaagacacgaugugag | |
| | | | | | ccuauuuu | |
| V16 | S1 | Alt V5 | guuucaaaaccgaugaaaaucggu | 330 | GCUCGUGGGUUUGUG | 426 |
| | | | ccuugaaaagagcaucggucugaa | | GUUGCCguuucaaaaccgau | |
| | | | ggaugcacuccgggauagggcagu | | gaaaaucgguccuugaaaagagc | |
| | | | cccggcucuugcuguuuccccggu | | aucggucugaaggaugcacucc | |
| | | | aagaccucggaagcaaguccuucag | | gggauagggcagucccggcucu | |
| | | | caagucgaaagacacgaugugagcc | | ugcuguuuccccgguaagaccu | |
| | | | uauuuu | | cggaagcaaguccuucagcaagu | |
| | | | | | cgaaagacacgaugugagccuau | |
| | | | | | uuu | |
| V17 | S1 | Alt V6 | guuucaaaccgaugaaaaucgguc | 331 | GCUCGUGGGUUUGUG | 427 |
| | | | cuugaaaagagcaucggucugaag | | GUUGCCguuucaaaccgaug | |
| | | | gaugcacuccgggauagggcaguc | | aaaaucgguccuugaaaagagca | |
| | | | ccggcucuugcuguuuccccggua | | ucggucugaaggaugcacuccg | |
| | | | agaccucggaagcaaguccuucagc | | ggauagggcagucccggcucuu | |
| | | | aagucgaaagacacgaugugagcc | | gcuguuuccccgguaagaccuc | |
| | | | uauuuu | | ggaagcaaguccuucagcaaguc | |
| | | | | | gaaagacacgaugugagccuauu | |
| | | | | | uu | |
| V18 | S1 | Alt V7 | guuucaaaccgaugaaaaucgguc | 332 | GCUCGUGGGUUUGUG | 428 |
| | | | cgguugaaaagagcaucggucuga | | GUUGCCguuucaaaccgaug | |
| | | | aggaugcacuccgggauagggcag | | aaaaucgguccgguugaaaagag | |
| | | | ucccggcucuugcuguuuccccgg | | caucggucugaaggaugcacucc | |
| | | | uaagaccucggaagcaaguccuuca | | gggauagggcagucccggcucu | |
| | | | gcaagucgaaagacacgaugugag | | ugcuguuuccccgguaagaccu | |
| | | | ccuauuuu | | cggaagcaaguccuucagcaagu | |
| | | | | | cgaaagacacgaugugagccuau | |
| | | | | | uuu | |
| V19 | S1 | Alt V8 | guuucaaaccgaugaaaaucgguc | 333 | GCUCGUGGGUUUGUG | 429 |
| | | | cUGAuugaaaagagcaucggucu | | GUUGCCguuucaaaccgaug | |
| | | | gaaggaugcacuccgggauagggc | | aaaaucgguccUGAuugaaaa | |
| | | | agucccggcucuugcuguuucccc | | gagcaucggucugaaggaugca | |
| | | | gguaagaccucggaagcaaguccu | | cuccgggauagggcagucccgg | |
| | | | ucagcaagucgaaagacacgaugu | | cucuugcuguuuccccgguaag | |
| | | | gagccuauuuu | | accucggaagcaaguccuucagc | |
| | | | | | aagucgaaagacacgaugugagc | |
| | | | | | cuauuuu | |
| V20 | S1 | Alt V9 | guuucaaucaaccgauagaaauauc | 334 | GCUCGUGGGUUUGUG | 430 |
| | | | gguUgguugaaaagagcaucggu | | GUUGCCguuucaaucaaccg | |
| | | | cugaaggaugcacuccgggauagg | | auagaaauaucgguUgguugaa | |
| | | | gcagucccggcucuugcuguuucc | | aagagcaucggucugaaggaug | |
| | | | ccgguaagaccucggaagcaagucc | | cacuccgggauagggcagucccg | |
| | | | uucagcaagucgaaagacacgaug | | gcucuugcuguuuccccgguaa | |
| | | | ugagccuauuuu | | gaccucggaagcaaguccuucag | |
| | | | | | caagucgaaagacacgaugugag | |
| | | | | | ccuauuuu | |
| V21 | S1 | Alt | guuucaaucGaccgauagaaauau | 335 | GCUCGUGGGUUUGUG | 431 |
| | | V10 | cgguccgguugaaaagagcaucgg | | GUUGCCguuucaaucGacc | |
| | | | ucugaaggaugcacuccgggauag | | gauagaaauaucgguccgguug | |
| | | | ggcagucccggcucuugcuguuuc | | aaaagagcaucggucugaaggau | |
| | | | cccgguaagaccucggaagcaaguc | | gcacuccgggauagggcaguccc | |
| | | | cuucagcaagucgaaagacacgaug | | ggcucuugcuguuuccccggua | |
| | | | ugagccuauuuu | | agaccucggaagcaaguccuuca | |
| | | | | | gcaagucgaaagacacgauguga | |
| | | | | | gccuauuuu | |
| V22 | S1 | Alt | guuucaaucaaccgauagaaauauc | 336 | GCUCGUGGGUUUGUG | 432 |
| | | V11 | gguUcgguugaaaagagcaucgg | | GUUGCCguuucaaucaaccg | |
| | | | ucugaaggaugcacuccgggauag | | auagaaauaucgguUcgguuga | |
| | | | ggcagucccggcucuugcuguuuc | | aaagagcaucggucugaaggau | |
| | | | cccgguaagaccucggaagcaaguc | | gcacuccgggauagggcaguccc | |
| | | | cuucagcaagucgaaagacacgaug | | ggcucuugcuguuuccccggua | |
| | | | ugagccuauuuu | | agaccucggaagcaaguccuuca | |
| | | | | | gcaagucgaaagacacgauguga | |
| | | | | | gccuauuuu | |
| V23 | S1 | Alt | guuucaaucaaccgauagaaauauc | 337 | GCUCGUGGGUUUGUG | 433 |
| | | V12 | ggucUgguugaaaagagcaucgg | | GUUGCCguuucaaucaaccg | |
| | | | ucugaaggaugcacuccgggauag | | auagaaauaucggucUgguuga | |
| | | | ggcagucccggcucuugcuguuuc | | aaagagcaucggucugaaggau | |
| | | | cccgguaagaccucggaagcaaguc | | gcacuccgggauagggcaguccc | |
| | | | cuucagcaagucgaaagacacgaug | | ggcucuugcuguuuccccggua | |
| | | | ugagccuauuuu | | agaccucggaagcaaguccuuca | |
| | | | | | gcaagucgaaagacacgauguga | |
| | | | | | gccuauuuu | |
| V24 | S1 | Alt | guuucaaucGaccgauagaaauau | 338 | GCUCGUGGGUUUGUG | 434 |
| | | V13 | cggucgguugaaaagagcaucggu | | GUUGCCguuucaaucGacc | |
| | | | cugaaggaugcacuccgggauagg | | gauagaaauaucggucgguuga | |
| | | | gcagucccggcucuugcuguuucc | | aaagagcaucggucugaaggau | |
| | | | ccgguaagaccucggaagcaagucc | | gcacuccgggauagggcaguccc | |
| | | | uucagcaagucgaaagacacgaug | | ggcucuugcuguuuccccggua | |
| | | | ugagccuauuuu | | agaccucggaagcaaguccuuca | |
| | | | | | gcaagucgaaagacacgauguga | |
| | | | | | gccuauuuu | |
| V25 | S1 | Alt | uuucaaucaaccgauagaaauaucg | 339 | GCUCGUGGGUUUGUG | 435 |
| | | V14 | guccgguugaaaagagcaucgguc | | GUUGCCuuucaaucaaccga | |
| | | | ugaaggaugcacuccgggauaggg | | uagaaauaucgguccgguugaa | |
| | | | cagucccggcucuugcuguuuccc | | aagagcaucggucugaaggaug | |
| | | | cgguaagaccucggaagcaagucc | | cacuccgggauagggcagucccg | |
| | | | uucagcaagucgaaagacacgaug | | gcucuugcuguuuccccgguaa | |
| | | | ugagcuauuuu | | gaccucggaagcaaguccuucag | |
| | | | | | caagucgaaagacacgaugugag | |
| | | | | | ccuauuuu | |
| V26 | S1 | Alt | guuucaaucaaccgauagaaauauc | 340 | GCUCGUGGGUUUGUG | 436 |
| | | V15 | gguccgguugaaaUgcaucgguc | | GUUGCCguuucaaucaaccg | |
| | | | ugaaggaugcacuccgggauaggg | | auagaaauaucgguccgguuga | |
| | | | cagucccggcucuugcuguuuccc | | aaUgcaucggucugaaggaugc | |
| | | | cgguaagaccucggaagcaagucc | | acuccgggauagggcagucccg | |
| | | | uucagcaagucgaaagacacgaug | | gcucuugcuguuuccccgguaa | |
| | | | ugagccuauuuu | | gaccucggaagcaaguccuucag | |
| | | | | | caagucgaaagacacgaugugag | |
| | | | | | ccuauuuu | |
| V27 | 3′-end | Del 1- | guuucaaucaaccgauagaaauauc | 341 | GCUCGUGGGUUUGUG | 437 |
| | | nt | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuucc | | aaagagcaucggucugaaggau | |
| | | | ccgguaagaccucggaagcaagucc | | gcacuccgggauagggcaguccc | |
| | | | uucagcaagucgaaagacacgaug | | ggcucuugcuguuuccccggua | |
| | | | ugagccuauuu | | agaccucggaagcaaguccuuca | |
| | | | | | gcaagucgaaagacacgauguga | |
| | | | | | gccuauuu | |
| V28 | 3′-end | Del 2- | guuucaaucaaccgauagaaauauc | 342 | GCUCGUGGGUUUGUG | 438 |
| | | nt | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuucc | | aaagagcaucggucugaaggau | |
| | | | ccgguaagaccucggaagcaagucc | | gcacuccgggauagggcaguccc | |
| | | | uucagcaagucgaaagacacgaug | | ggcucuugcuguuuccccggua | |
| | | | ugagccuauu | | agaccucggaagcaaguccuuca | |
| | | | | | gcaagucgaaagacacgauguga | |
| | | | | | gccuauu | |
| V29 | 3′-end | Del 3- | guuucaaucaaccgauagaaauauc | 343 | GCUCGUGGGUUUGUG | 439 |
| | | nt | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuucc | | aaagagcaucggucugaaggau | |
| | | | ccgguaagaccucggaagcaagucc | | gcacuccgggauagggcaguccc | |
| | | | uucagcaagucgaaagacacgaug | | ggcucuugcuguuuccccggua | |
| | | | ugagccuau | | agaccucggaagcaaguccuuca | |
| | | | | | gcaagucgaaagacacgauguga | |
| | | | | | gccuau | |
| V30 | 3′-end | Del 4- | guuucaaucaaccgauagaaauauc | 344 | GCUCGUGGGUUUGUG | 440 |
| | | nt | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuucc | | aaagagcaucggucugaaggau | |
| | | | ccgguaagaccucggaagcaagucc | | gcacuccgggauagggcaguccc | |
| | | | uucagcaagucgaaagacacgaug | | ggcucuugcuguuuccccggua | |
| | | | ugagccua | | agaccucggaagcaaguccuuca | |
| | | | | | gcaagucgaaagacacgauguga | |
| | | | | | gccua | |
| V31 | 3′-end | Del 5- | guuucaaucaaccgauagaaauauc | 345 | GCUCGUGGGUUUGUG | 441 |
| | | nt | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuucc | | aaagagcaucggucugaaggau | |
| | | | ccgguaagaccucggaagcaagucc | | gcacuccgggauagggcaguccc | |
| | | | uucagcaagucgaaagacacgaug | | ggcucuugcuguuuccccggua | |
| | | | ugagccu | | agaccucggaagcaaguccuuca | |
| | | | | | gcaagucgaaagacacgauguga | |
| | | | | | gccu | |
| V32 | 3′-end | Del 6- | guuucaaucaaccgauagaaauauc | 346 | GCUCGUGGGUUUGUG | 442 |
| | | nt | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuucc | | aaagagcaucggucugaaggau | |
| | | | ccgguaagaccucggaagcaagucc | | gcacuccgggauagggcaguccc | |
| | | | uucagcaagucgaaagacacgaug | | ggcucuugcuguuuccccggua | |
| | | | ugagcc | | agaccucggaagcaaguccuuca | |
| | | | | | gcaagucgaaagacacgauguga | |
| | | | | | gcc | |
| V33 | 3′-end | Del 7- | guuucaaucaaccgauagaaauauc | 347 | GCUCGUGGGUUUGUG | 443 |
| | | nt | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuucc | | aaagagcaucggucugaaggau | |
| | | | ccgguaagaccucggaagcaagucc | | gcacuccgggauagggcaguccc | |
| | | | uucagcaagucgaaagacacgaug | | ggcucuugcuguuuccccggua | |
| | | | ugagc | | agaccucggaagcaaguccuuca | |
| | | | | | gcaagucgaaagacacgauguga | |
| | | | | | gc | |
| V34 | 3′-end | Del 8- | guuucaaucaaccgauagaaauauc | 348 | GCUCGUGGGUUUGUG | 444 |
| | | nt | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuucc | | aaagagcaucggucugaaggau | |
| | | | ccgguaagaccucggaagcaagucc | | gcacuccgggauagggcaguccc | |
| | | | uucagcaagucgaaagacacgaug | | ggcucuugcuguuuccccggua | |
| | | | ugag | | agaccucggaagcaaguccuuca | |
| | | | | | gcaagucgaaagacacgaugugag | |
| V35 | 3′-end | Del 9- | guuucaaucaaccgauagaaauauc | 349 | GCUCGUGGGUUUGUG | 445 |
| | | nt | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuucc | | aaagagcaucggucugaaggau | |
| | | | ccgguaagaccucggaagcaagucc | | gcacuccgggauagggcaguccc | |
| | | | uucagcaagucgaaagacacgaug | | ggcucuugcuguuuccccggua | |
| | | | uga | | agaccucggaagcaaguccuuca | |
| | | | | | gcaagucgaaagacacgauguga | |
| V36 | 3′-end | Del 10- | guuucaaucaaccgauagaaauauc | 350 | GCUCGUGGGUUUGUG | 446 |
| | | nt | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuucc | | aaagagcaucggucugaaggau | |
| | | | ccgguaagaccucggaagcaagucc | | gcacuccgggauagggcaguccc | |
| | | | uucagcaagucgaaagacacgaug | | ggcucuugcuguuuccccggua | |
| | | | ug | | agaccucggaagcaaguccuuca | |
| | | | | | gcaagucgaaagacacgaugug | |
| V37 | 3′-end | Del 11- | guuucaaucaaccgauagaaauauc | 351 | GCUCGUGGGUUUGUG | 447 |
| | | nt | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuucc | | aaagagcaucggucugaaggau | |
| | | | ccgguaagaccucggaagcaagucc | | gcacuccgggauagggcaguccc | |
| | | | uucagcaagucgaaagacacgaugu | | ggcucuugcuguuuccccggua | |
| | | | | | agaccucggaagcaaguccuuca | |
| | | | | | gcaagucgaaagacacgaugu | |
| V38 | S3 | Del | guuucaaucaaccgauagaaauauc | 352 | GCUCGUGGGUUUGUG | 448 |
| | | addi- | gguccgguugaaaagagcaucggc | | GUUGCCguuucaaucaaccg | |
| | | tioinal | ugaaggaugcacuccgggauaggg | | auagaaauaucgguccgguuga | |
| | | U | cagucccggcucuugcuguuuccc | | aaagagcaucggcugaaggaugc | |
| | | | cgguaagaccucggaagcaagucc | | acuccgggauagggcagucccg | |
| | | | uucagcaagucgaaagacacgaug | | gcucuugcuguuuccccgguaa | |
| | | | ugagccuauuuu | | gaccucggaagcaaguccuucag | |
| | | | | | caagucgaaagacacgaugugag | |
| | | | | | ccuauuuu | |
| V39 | S6 | Del 1- | guuucaaucaaccgauagaaauauc | 353 | GCUCGUGGGUUUGUG | 449 |
| | | bp | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuucc | | aaagagcaucggucugaaggau | |
| | | | ccgguaagaccucggaagcaagucc | | gcacuccgggauagggcaguccc | |
| | | | uucagcaagugaaaacacgaugug | | ggcucuugcuguuuccccggua | |
| | | | agagccuauuuu | | agaccucggaagcaaguccuuca | |
| | | | | | gcaagugaaaacacgaugugagc | |
| | | | | | cuauuuu | |
| V40 | S6 | Del 2- | guuucaaucaaccgauagaaauauc | 354 | GCUCGUGGGUUUGUG | 450 |
| | | bp | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuucc | | aaagagcaucggucugaaggau | |
| | | | ccgguaagaccucggaagcaagucc | | gcacuccgggauagggcaguccc | |
| | | | uucagcaaggaaacacgaugugag | | ggcucuugcuguuuccccggua | |
| | | | ccuauuuu | | agaccucggaagcaaguccuuca | |
| | | | | | gcaaggaaacacgaugugagccu | |
| | | | | | auuuu | |
| V41 | S6 | Del 3- | guuucaaucaaccgauagaaauauc | 355 | GCUCGUGGGUUUGUG | 451 |
| | | bp | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuucc | | aaagagcaucggucugaaggau | |
| | | | ccgguaagaccucggaagcaagucc | | gcacuccgggauagggcaguccc | |
| | | | uucagcaagaaaacgaugugagcc | | ggcucuugcuguuuccccggua | |
| | | | uauuuu | | agaccucggaagcaaguccuuca | |
| | | | | | gcaagaaaacgaugugagccuau | |
| | | | | | uuu | |
| V42 | S6 | Del 4- | guuucaaucaaccgauagaaauauc | 356 | GCUCGUGGGUUUGUG | 452 |
| | | bp | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuucc | | aaagagcaucggucugaaggau | |
| | | | ccgguaagaccucggaagcaagucc | | gcacuccgggauagggcaguccc | |
| | | | uucagcagaaaacgaugugagccu | | ggcucuugcuguuuccccggua | |
| | | | auuuu | | agaccucggaagcaaguccuuca | |
| | | | | | gcagaaaacgaugugagccuauu | |
| | | | | | uu | |
| V43 | S6 | Alt V1 | guuucaaucaaccgauagaaauauc | 357 | GCUCGUGGGUUUGUG | 453 |
| | | | gguccgguugaaaagagcaucggc | | GUUGCCguuucaaucaaccg | |
| | | | ugaaggaugcacuccgggauaggg | | auagaaauaucgguccgguuga | |
| | | | cagucccggcucuugcuguuuccc | | aaagagcaucggcugaaggaugc | |
| | | | cgguaagaccucggaagcaagucc | | acuccgggauagggcagucccg | |
| | | | uucagcAcgaugugagccuauuuu | | gcucuugcuguuuccccgguaa | |
| | | | | | gaccucggaagcaaguccuucag | |
| | | | | | cAcgaugugagccuauuuu | |
| V44 | S6 | Alt V2 | guuucaaucaaccgauagaaauauc | 358 | GCUCGUGGGUUUGUG | 454 |
| | | | gguccgguugaaaagagcaucggc | | GUUGCCguuucaaucaaccg | |
| | | | ugaaggaugcacuccgggauaggg | | auagaaauaucgguccgguuga | |
| | | | cagucccggcucuugcuguuuccc | | aaagagcaucggcugaaggaugc | |
| | | | cgguaagaccucggaagcaagucc | | acuccgggauagggcagucccg | |
| | | | uucagcUUAcgaugugagccuau | | gcucuugcuguuuccccgguaa | |
| | | | uuu | | gaccucggaagcaaguccuucag | |
| | | | | | cUUAcgaugugagccuauuuu | |
| V45 | S6 | Alt V3 | guuucaaucaaccgauagaaauauc | 359 | GCUCGUGGGUUUGUG | 455 |
| | | | gguccgguugaaaagagcaucggc | | GUUGCCguuucaaucaaccg | |
| | | | ugaaggaugcacuccgggauaggg | | auagaaauaucgguccgguuga | |
| | | | cagucccggcucuugcuguuuccc | | aaagagcaucggcugaaggaugc | |
| | | | cgguaagaccucggaagcaagucc | | acuccgggauagggcagucccg | |
| | | | uucagcGAGCcgaugugagccu | | gcucuugcuguuuccccgguaa | |
| | | | auuuu | | gaccucggaagcaaguccuucag | |
| | | | | | cGAGCcgaugugagccuauu | |
| | | | | | uu | |
| V46 | S6 | Alt V4 | guuucaaucaaccgauagaaauauc | 360 | GCUCGUGGGUUUGUG | 456 |
| | | | gguccgguugaaaagagcaucgAg | | GUUGCCguuucaaucaaccg | |
| | | | cugaaggaugcacuccgggauagg | | auagaaauaucgguccgguuga | |
| | | | gcagucccggcucuugcuguuucc | | aaagagcaucgAgcugaaggau | |
| | | | ccgguaagaccucggaagcaagucc | | gcacuccgggauagggcaguccc | |
| | | | uucagcGAGCcgaugugagccu | | ggcucuugcuguuuccccggua | |
| | | | auuuu | | agaccucggaagcaaguccuuca | |
| | | | | | gcGAGCcgaugugagccuau | |
| | | | | | uuu | |
| V47 | S6 | Alt V5 | guuucaaucaaccgauagaaauauc | 361 | GCUCGUGGGUUUGUG | 457 |
| | | | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuucc | | aaagagcaucggucugaaggau | |
| | | | ccgguaagaccucggaagcaagucc | | gcacuccgggauagggcaguccc | |
| | | | uucagccgaugugagccuauuuu | | ggcucuugcuguuuccccggua | |
| | | | | | agaccucggaagcaaguccuuca | |
| | | | | | gccgaugugagccuauuuu | |
| V48 | S6 | Alt V6 | guuucaaucaaccgauagaaauauc | 362 | GCUCGUGGGUUUGUG | 458 |
| | | | gguccgguugaaaagagcaucggc | | GUUGCCguuucaaucaaccg | |
| | | | ugaaggaugcacuccgggauaggg | | auagaaauaucgguccgguuga | |
| | | | cagucccggcucuugcuguuuccc | | aaagagcaucggcugaaggaugc | |
| | | | cgguaagaccucggaagcaagucc | | acuccgggauagggcagucccg | |
| | | | uucagccgaugugagccuauuuu | | gcucuugcuguuuccccgguaa | |
| | | | | | gaccucggaagcaaguccuucag | |
| | | | | | ccgaugugagccuauuuu | |
| V49 | S4 | Del 1- | Guuucaaucaaccgauagaaauau | 363 | GCUCGUGGGUUUGUG | 459 |
| | | bp | cgguccgguugaaaagagcaucgg | | GUUGCCGuuucaaucaacc | |
| | | | ucugaaggaugcacuccgggaagg | | gauagaaauaucgguccgguug | |
| | | | gcaucccggcucuugcuguuuccc | | aaaagagcaucggucugaaggau | |
| | | | cgguaagaccucggaagcaagucc | | gcacuccgggaagggcaucccgg | |
| | | | uucagcaagucgaaagacacgaug | | cucuugcuguuuccccgguaag | |
| | | | ugagccuauuuu | | accucggaagcaaguccuucagc | |
| | | | | | aagucgaaagacacgaugugagc | |
| | | | | | cuauuuu | |
| V50 | S4 | Del 2- | Guuucaaucaaccgauagaaauau | 364 | GCUCGUGGGUUUGUG | 460 |
| | | bp | cgguccgguugaaaagagcaucgg | | GUUGCCGuuucaaucaacc | |
| | | | ucugaaggaugcacuccgggaggg | | gauagaaauaucgguccgguug | |
| | | | cacccggcucuugcuguuuccccg | | aaaagagcaucggucugaaggau | |
| | | | guaagaccucggaagcaaguccuu | | gcacuccgggagggcacccggcu | |
| | | | cagcaagucgaaagacacgauguga | | cuugcuguuuccccgguaagac | |
| | | | gccuauuuu | | cucggaagcaaguccuucagcaa | |
| | | | | | gucgaaagacacgaugugagccu | |
| | | | | | auuuu | |
| V51 | S4 | Del 3- | Guuucaaucaaccgauagaaauau | 365 | GCUCGUGGGUUUGUG | 461 |
| | | bp | cgguccgguugaaaagagcaucgg | | GUUGCCGuuucaaucaacc | |
| | | | ucugaaggaugcacuccggagggc | | gauagaaauaucgguccgguug | |
| | | | accggcucuugcuguuuccccggu | | aaaagagcaucggucugaaggau | |
| | | | aagaccucggaagcaaguccuucag | | gcacuccggagggcaccggcucu | |
| | | | caagucgaaagacacgaugugagcc | | ugcuguuuccccgguaagaccu | |
| | | | uauuuu | | cggaagcaaguccuucagcaagu | |
| | | | | | cgaaagacacgaugugagccuau | |
| | | | | | uuu | |
| V52 | S4 | Del 4- | Guuucaaucaaccgauagaaauau | 366 | GCUCGUGGGUUUGUG | 462 |
| | | bp | cgguccgguugaaaagagcaucgg | | GUUGCCGuuucaaucaacc | |
| | | | ucugaaggaugcacuccgagggca | | gauagaaauaucgguccgguug | |
| | | | cggcucuugcuguuuccccgguaa | | aaaagagcaucggucugaaggau | |
| | | | gaccucggaagcaaguccuucagca | | gcacuccgagggcacggcucuu | |
| | | | agucgaaagacacgaugugagccu | | gcuguuuccccgguaagaccuc | |
| | | | auuuu | | ggaagcaaguccuucagcaaguc | |
| | | | | | gaaagacacgaugugagccuauu | |
| | | | | | uu | |
| V53 | S4 | Del 5- | Guuucaaucaaccgauagaaauau | 367 | GCUCGUGGGUUUGUG | 463 |
| | | bp | cgguccgguugaaaagagcaucgg | | GUUGCCGuuucaaucaacc | |
| | | | ucugaaggaugcacuccagggcag | | gauagaaauaucgguccgguug | |
| | | | gcucuugcuguuuccccgguaaga | | aaaagagcaucggucugaaggau | |
| | | | ccucggaagcaaguccuucagcaag | | gcacuccagggcaggcucuugc | |
| | | | ucgaaagacacgaugugagccuau | | uguuuccccgguaagaccucgg | |
| | | | uuu | | aagcaaguccuucagcaagucga | |
| | | | | | aagacacgaugugagccuauuuu | |
| V54 | S4 | Del 6- | Guuucaaucaaccgauagaaauau | 368 | GCUCGUGGGUUUGUG | 464 |
| | | bp | cgguccgguugaaaagagcaucgg | | GUUGCCGuuucaaucaacc | |
| | | | ucugaaggaugcacucagggcagc | | gauagaaauaucgguccgguug | |
| | | | ucuugcuguuuccccgguaagacc | | aaaagagcaucggucugaaggau | |
| | | | ucggaagcaaguccuucagcaagu | | gcacucagggcagcucuugcug | |
| | | | cgaaagacacgaugugagccuauu | | uuuccccgguaagaccucggaag | |
| | | | uu | | caaguccuucagcaagucgaaag | |
| | | | | | acacgaugugagccuauuuu | |
| V55 | S4 | Del 7- | Guuucaaucaaccgauagaaauau | 369 | GCUCGUGGGUUUGUG | 465 |
| | | bp | cgguccgguugaaaagagcaucgg | | GUUGCCGuuucaaucaacc | |
| | | | ucugaaggaugcacuagggcacuc | | gauagaaauaucgguccgguug | |
| | | | uugcuguuuccccgguaagaccuc | | aaaagagcaucggucugaaggau | |
| | | | ggaagcaaguccuucagcaagucg | | gcacuagggcacucuugcuguu | |
| | | | aaagacacgaugugagccuauuuu | | uccccgguaagaccucggaagca | |
| | | | | | aguccuucagcaagucgaaagac | |
| | | | | | acgaugugagccuauuuu | |
| V56 | S4 | Del 9- | Guuucaaucaaccgauagaaauau | 370 | GCUCGUGGGUUUGUG | 466 |
| | | bp | cgguccgguugaaaagagcaucgg | | GUUGCCGuuucaaucaacc | |
| | | | ucugaaggaugcaagggcaugcug | | gauagaaauaucgguccgguug | |
| | | | uuuccccgguaagaccucggaagc | | aaaagagcaucggucugaaggau | |
| | | | aaguccuucagcaagucgaaagaca | | gcaagggcaugcuguuuccccg | |
| | | | cgaugugagccuauuuu | | guaagaccucggaagcaaguccu | |
| | | | | | ucagcaagucgaaagacacgaug | |
| | | | | | ugagccuauuuu | |
| V57 | S4 | Del 10- | Guuucaaucaaccgauagaaauau | 371 | GCUCGUGGGUUUGUG | 467 |
| | | bp | cgguccgguugaaaagagcaucgg | | GUUGCCGuuucaaucaacc | |
| | | | ucugaaggaugcagggcagcuguu | | gauagaaauaucgguccgguug | |
| | | | uccccgguaagaccucggaagcaag | | aaaagagcaucggucugaaggau | |
| | | | uccuucagcaagucgaaagacacga | | gcagggcagcuguuuccccggu | |
| | | | ugugagccuauuuu | | aagaccucggaagcaaguccuuc | |
| | | | | | agcaagucgaaagacacgaugug | |
| | | | | | agccuauuuu | |
| V58 | S4 | Del 11- | guuucaaucaaccgauagaaauauc | 372 | GCUCGUGGGUUUGUG | 468 |
| | | bp | gguccgguugaaaagagcaucggu | | GUUGCCguuucaaucaaccg | |
| | | | cugaaggaugagggcacuguuucc | | auagaaauaucgguccgguuga | |
| | | | ccgguaagaccucggaagcaagucc | | aaagagcaucggucugaaggau | |
| | | | uucagcaagucgaaagacacgaug | | gagggcacuguuuccccgguaa | |
| | | | ugagccuauuuu | | gaccucggaagcaaguccuucag | |
| | | | | | caagucgaaagacacgaugugag | |
| | | | | | ccuauuuu | |
| V59 | S4 | Del 11- | guuucaaucaaccgauagaaauauc | 373 | GCUCGUGGGUUUGUG | 469 |
| | | bp, No | gguccgguugaaaagagcaucggu | | GUUGCCguuucaaucaaccg | |
| | | loop | cugaaggaugcuguuuccccggua | | auagaaauaucgguccgguuga | |
| | | | agaccucggaagcaaguccuucagc | | aaagagcaucggucugaaggau | |
| | | | aagucgaaagacacgaugugagcc | | gcuguuuccccgguaagaccuc | |
| | | | uauuuu | | ggaagcaaguccuucagcaaguc | |
| | | | | | gaaagacacgaugugagccuauu | |
| | | | | | uu | |
| V60 | S5 | Del 1- | guuucaaucaaccgauagaaauauc | 374 | GCUCGUGGGUUUGUG | 470 |
| | | bp | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuucc | | aaagagcaucggucugaaggau | |
| | | | ccggaagccucggaagcaaguccu | | gcacuccgggauagggcaguccc | |
| | | | ucagcaagucgaaagacacgaugu | | ggcucuugcuguuuccccggaa | |
| | | | gagccuauuuu | | gccucggaagcaaguccuucagc | |
| | | | | | aagucgaaagacacgaugugagc | |
| | | | | | cuauuuu | |
| V61 | S5 | Del 2- | guuucaaucaaccgauagaaauauc | 375 | GCUCGUGGGUUUGUG | 471 |
| | | bp | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuucc | | aaagagcaucggucugaaggau | |
| | | | ccgaagcucggaagcaaguccuuca | | gcacuccgggauagggcaguccc | |
| | | | gcaagucgaaagacacgaugugag | | ggcucuugcuguuuccccgaag | |
| | | | ccuauuuu | | cucggaagcaaguccuucagcaa | |
| | | | | | gucgaaagacacgaugugagccu | |
| | | | | | auuuu | |
| V62 | S5 | Del 3- | guuucaaucaaccgauagaaauauc | 376 | GCUCGUGGGUUUGUG | 472 |
| | | bp | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuucc | | aaagagcaucggucugaaggau | |
| | | | ccaagucggaagcaaguccuucagc | | gcacuccgggauagggcaguccc | |
| | | | aagucgaaagacacgaugugagcc | | ggcucuugcuguuuccccaagu | |
| | | | uauuuu | | cggaagcaaguccuucagcaagu | |
| | | | | | cgaaagacacgaugugagccuau | |
| | | | | | uuu | |
| V63 | S5 | Del 5- | guuucaaucaaccgauagaaauauc | 377 | GCUCGUGGGUUUGUG | 473 |
| | | bp | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuucc | | aaagagcaucggucugaaggau | |
| | | | aagggaagcaaguccuucagcaag | | gcacuccgggauagggcaguccc | |
| | | | ucgaaagacacgaugugagccuau | | ggcucuugcuguuuccaaggga | |
| | | | uuu | | agcaaguccuucagcaagucgaa | |
| | | | | | agacacgaugugagccuauuuu | |
| V64 | S5 | Del 6- | guuucaaucaaccgauagaaauauc | 378 | GCUCGUGGGUUUGUG | 474 |
| | | bp | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuuca | | aaagagcaucggucugaaggau | |
| | | | aggaagcaaguccuucagcaaguc | | gcacuccgggauagggcaguccc | |
| | | | gaaagacacgaugugagccuauuu | | ggcucuugcuguuucaaggaag | |
| | | | u | | caaguccuucagcaagucgaaag | |
| | | | | | acacgaugugagccuauuuu | |
| V65 | S5 | Del 7- | guuucaaucaaccgauagaaauauc | 379 | GCUCGUGGGUUUGUG | 475 |
| | | bp | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuuaa | | aaagagcaucggucugaaggau | |
| | | | gaagcaaguccuucagcaagucgaa | | gcacuccgggauagggcaguccc | |
| | | | agacacgaugugagccuauuuu | | ggcucuugcuguuuaagaagca | |
| | | | | | aguccuucagcaagucgaaagac | |
| | | | | | acgaugugagccuauuuu | |
| V66 | S5 | Del 9- | guuucaaucaaccgauagaaauauc | 380 | GCUCGUGGGUUUGUG | 476 |
| | | bp | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuaag | | aaagagcaucggucugaaggau | |
| | | | agcaaguccuucagcaagucgaaag | | gcacuccgggauagggcaguccc | |
| | | | acacgaugugagccuauuuu | | ggcucuugcuguuaagagcaag | |
| | | | | | uccuucagcaagucgaaagacac | |
| | | | | | gaugugagccuauuuu | |
| V67 | S5 | Del 10- | guuucaaucaaccgauagaaauauc | 381 | GCUCGUGGGUUUGUG | 477 |
| | | bp | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguaagg | | aaagagcaucggucugaaggau | |
| | | | caaguccuucagcaagucgaaagac | | gcacuccgggauagggcaguccc | |
| | | | acgaugugagccuauuuu | | ggcucuugcuguaaggcaaguc | |
| | | | | | cuucagcaagucgaaagacacga | |
| | | | | | ugugagccuauuuu | |
| V68 | S5 | Del 11- | guuucaaucaaccgauagaaauauc | 382 | GCUCGUGGGUUUGUG | 478 |
| | | bp | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcugaagca | | aaagagcaucggucugaaggau | |
| | | | aguccuucagcaagucgaaagacac | | gcacuccgggauagggcaguccc | |
| | | | gaugugagccuauuuu | | ggcucuugcugaagcaaguccu | |
| | | | | | ucagcaagucgaaagacacgaug | |
| | | | | | ugagccuauuuu | |
| V69 | S5 | Del 12- | guuucaaucaaccgauagaaauauc | 383 | GCUCGUGGGUUUGUG | 479 |
| | | bp | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuaagaag | | aaagagcaucggucugaaggau | |
| | | | uccuucagcaagucgaaagacacga | | gcacuccgggauagggcaguccc | |
| | | | ugugagccuauuuu | | ggcucuugcuaagaaguccuuc | |
| | | | | | agcaagucgaaagacacgaugug | |
| | | | | | agccuauuuu | |
| V70 | S5 | Del 12- | guuucaaucaaccgauagaaauauc | 384 | GCUCGUGGGUUUGUG | 480 |
| | | bp, No | gguccgguugaaaagagcaucggu | | GUUGCCguuucaaucaaccg | |
| | | loop | cugaaggaugcacuccgggauagg | | auagaaauaucgguccgguuga | |
| | | | gcagucccggcucuugcuaagucc | | aaagagcaucggucugaaggau | |
| | | | uucagcaagucgaaagacacgaug | | gcacuccgggauagggcaguccc | |
| | | | ugagccuauuuu | | ggcucuugcuaaguccuucagc | |
| | | | | | aagucgaaagacacgaugugagc | |
| | | | | | cuauuuu | |
| V71 | S5 | Alt V1 | guuucaaucaaccgauagaaauauc | 385 | GCUCGUGGGUUUGUG | 481 |
| | | | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuucc | | aaagagcaucggucugaaggau | |
| | | | GAgguaagaccucggaagcaagu | | gcacuccgggauagggcaguccc | |
| | | | ccuucagcaagucgaaagacacgau | | ggcucuugcuguuuccGAggu | |
| | | | gugagccuauuuu | | aagaccucggaagcaaguccuuc | |
| | | | | | agcaagucgaaagacacgaugug | |
| | | | | | agccuauuuu | |
| V72 | S5 | Alt V2 | guuucaaucaaccgauagaaauauc | 386 | GCUCGUGGGUUUGUG | 482 |
| | | | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuucc | | aaagagcaucggucugaaggau | |
| | | | ccgguaagaccGGggaagcaagu | | gcacuccgggauagggcaguccc | |
| | | | ccuucagcaagucgaaagacacgau | | ggcucuugcuguuuccccggua | |
| | | | gugagccuauuuu | | agaccGGggaagcaaguccuuc | |
| | | | | | agcaagucgaaagacacgaugug | |
| | | | | | agccuauuuu | |
| V73 | S5 | Alt V3 | guuucaaucaaccgauagaaauauc | 387 | GCUCGUGGGUUUGUG | 483 |
| | | | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuucc | | aaagagcaucggucugaaggau | |
| | | | Gcgguaagaccucggaagcaaguc | | gcacuccgggauagggcaguccc | |
| | | | cuucagcaagucgaaagacacgaug | | ggcucuugcuguuuccGcggua | |
| | | | ugagccuauuuu | | agaccucggaagcaaguccuuca | |
| | | | | | gcaagucgaaagacacgauguga | |
| | | | | | gccuauuuu | |
| V74 | S5 | Alt V4 | guuucaaucaaccgauagaaauauc | 388 | GCUCGUGGGUUUGUG | 484 |
| | | | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuucc | | aaagagcaucggucugaaggau | |
| | | | cAgguaagaccucggaagcaaguc | | gcacuccgggauagggcaguccc | |
| | | | cuucagcaagucgaaagacacgaug | | ggcucuugcuguuucccAggua | |
| | | | ugagccuauuuu | | agaccucggaagcaaguccuuca | |
| | | | | | gcaagucgaaagacacgauguga | |
| | | | | | gccuauuuu | |
| V75 | S5 | Alt V5 | guuucaaucaaccgauagaaauauc | 389 | GCUCGUGGGUUUGUG | 485 |
| | | | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuucc | | aaagagcaucggucugaaggau | |
| | | | ccgguaagaccGcggaagcaaguc | | gcacuccgggauagggcaguccc | |
| | | | cuucagcaagucgaaagacacgaug | | ggcucuugcuguuuccccggua | |
| | | | ugagccuauuuu | | agaccGcggaagcaaguccuuc | |
| | | | | | agcaagucgaaagacacgaugug | |
| | | | | | agccuauuuu | |
| V76 | S5 | Alt V6 | guuucaaucaaccgauagaaauauc | 390 | GCUCGUGGGUUUGUG | 486 |
| | | | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuucc | | aaagagcaucggucugaaggau | |
| | | | ccgguaagaccuGggaagcaaguc | | gcacuccgggauagggcaguccc | |
| | | | cuucagcaagucgaaagacacgaug | | ggcucuugcuguuuccccggua | |
| | | | ugagccuauuuu | | agaccuGggaagcaaguccuuc | |
| | | | | | agcaagucgaaagacacgaugug | |
| | | | | | agccuauuuu | |
| V77 | S5 | Alt V7 | guuucaaucaaccgauagaaauauc | 391 | GCUCGUGGGUUUGUG | 487 |
| | | | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuucc | | aaagagcaucggucugaaggau | |
| | | | gguaagaccGggaagcaaguccuu | | gcacuccgggauagggcaguccc | |
| | | | cagcaagucgaaagacacgauguga | | ggcucuugcuguuuccgguaag | |
| | | | gccuauuuu | | accGggaagcaaguccuucagc | |
| | | | | | aagucgaaagacacgaugugagc | |
| | | | | | cuauuuu | |
| V78 | S5 | Alt V8 | guuucaaucaaccgauagaaauauc | 392 | GCUCGUGGGUUUGUG | 488 |
| | | | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuucc | | aaagagcaucggucugaaggau | |
| | | | gguaagaccggaagcaaguccuuc | | gcacuccgggauagggcaguccc | |
| | | | agcaagucgaaagacacgauguga | | ggcucuugcuguuuccgguaag | |
| | | | gccuauuuu | | accggaagcaaguccuucagcaa | |
| | | | | | gucgaaagacacgaugugagccu | |
| | | | | | auuuu | |
| V79 | S5 | Alt V9 | guuucaaucaaccgauagaaauauc | 393 | GCUCGUGGGUUUGUG | 489 |
| | | | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuucc | | aaagagcaucggucugaaggau | |
| | | | ccgguGAAAaccucggaagcaa | | gcacuccgggauagggcaguccc | |
| | | | guccuucagcaagucgaaagacacg | | ggcucuugcuguuuccccgguG | |
| | | | augugagccuauuuu | | AAAaccucggaagcaaguccu | |
| | | | | | ucagcaagucgaaagacacgaug | |
| | | | | | ugagccuauuuu | |
| V80 | S5 | Alt | guuucaaucaaccgauagaaauauc | 394 | GCUCGUGGGUUUGUG | 490 |
| | | V10 | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuucc | | aaagagcaucggucugaaggau | |
| | | | ccggGAAAccucggaagcaagu | | gcacuccgggauagggcaguccc | |
| | | | ccuucagcaagucgaaagacacgau | | ggcucuugcuguuuccccggG | |
| | | | gugagccuauuuu | | AAAccucggaagcaaguccuu | |
| | | | | | cagcaagucgaaagacacgaugu | |
| | | | | | gagccuauuuu | |
| V81 | S5 | Alt | guuucaaucaaccgauagaaauauc | 395 | GCUCGUGGGUUUGUG | 491 |
| | | V11 | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuucc | | aaagagcaucggucugaaggau | |
| | | | ccgGAAAcucggaagcaagucc | | gcacuccgggauagggcaguccc | |
| | | | uucagcaagucgaaagacacgaug | | ggcucuugcuguuuccccgGA | |
| | | | ugagccuauuuu | | AAcucggaagcaaguccuucag | |
| | | | | | caagucgaaagacacgaugugag | |
| | | | | | ccuauuuu | |
| V82 | S5 | Alt | guuucaaucaaccgauagaaauauc | 396 | GCUCGUGGGUUUGUG | 492 |
| | | V12 | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuucc | | aaagagcaucggucugaaggau | |
| | | | ccGAAAucggaagcaaguccuu | | gcacuccgggauagggcaguccc | |
| | | | cagcaagucgaaagacacgauguga | | ggcucuugcuguuuccccGAA | |
| | | | gccuauuuu | | Aucggaagcaaguccuucagca | |
| | | | | | agucgaaagacacgaugugagcc | |
| | | | | | uauuuu | |
| V83 | S1 | G:U to | guuucaaCcaaccgauagaaauau | 397 | GCUCGUGGGUUUGUG | 493 |
| | | G:C/A:U | cgguccgguugaaaagagcaucgg | | GUUGCCguuucaaCcaacc | |
| | | | ucugaaggaugcacuccgggauag | | gauagaaauaucgguccgguug | |
| | | | ggcagucccggcucuugcuguuuc | | aaaagagcaucggucugaaggau | |
| | | | cccgguaagaccucggaagcaaguc | | gcacuccgggauagggcaguccc | |
| | | | cuucagcaagucgaaagacacgaug | | ggcucuugcuguuuccccggua | |
| | | | ugagccuauuuu | | agaccucggaagcaaguccuuca | |
| | | | | | gcaagucgaaagacacgauguga | |
| | | | | | gccuauuuu | |
| V84 | S1 | G:U to | guuucaaucaaccgauagaaauauc | 398 | GCUCGUGGGUUUGUG | 494 |
| | | G:C/A:U | gguccAguugaaaagagcaucggu | | GUUGCCguuucaaucaaccg | |
| | | | cugaaggaugcacuccgggauagg | | auagaaauaucgguccAguuga | |
| | | | gcagucccggcucuugcuguuucc | | aaagagcaucggucugaaggau | |
| | | | ccgguaagaccucggaagcaagucc | | gcacuccgggauagggcaguccc | |
| | | | uucagcaagucgaaagacacgaug | | ggcucuugcuguuuccccgua | |
| | | | ugagccuauuuu | | agaccucggaagcaaguccuuca | |
| | | | | | gcaagucgaaagacacgauguga | |
| | | | | | gccuauuuu | |
| V85 | S1 | G:U to | guuucaaGcaaccgauagaaauau | 399 | GCUCGUGGGUUUGUG | 495 |
| | | G:C/A: | cgguccCguugaaaagagcaucgg | | GUUGCCguuucaaGcaacc | |
| | | U | ucugaaggaugcacuccgggauag | | gauagaaauaucgguccCguug | |
| | | | ggcagucccggcucuugcuguuuc | | aaaagagcaucggucugaaggau | |
| | | | cccgguaagaccucggaagcaguc | | gcacuccgggauagggcaguccc | |
| | | | cuucagcaagucgaaagacacgaug | | ggcucuugcuuuuccccgua | |
| | | | ugagccuauuuu | | agaccucggaagcaaguccuuca | |
| | | | | | gcaagucgaaagacacgauguga | |
| | | | | | gccuauuuu | |
| V86 | S2 | G:U to | guuucaaucaaccgauagaaauauc | 400 | GCUCGUGGGUUUGUG | 496 |
| | | G:C/A:U | gguccgguugaaaagaAcaucggu | | GUUGCCguuucaaucaaccg | |
| | | | cugaaggaugcacuccgggauagg | | auagaaauaucgguccgguuga | |
| | | | gcagucccggcucuugcuguuucc | | aaagaAcaucggucugaaggau | |
| | | | ccgguaagaccucggaagcaagucc | | gcacuccgggauagggcaguccc | |
| | | | uucagcaagucgaaagacacgaug | | ggcucuugcuguuuccccggua | |
| | | | ugagccuauuuu | | agaccucggaagcaaguccuuca | |
| | | | | | gcaagucgaaagacacgauguga | |
| | | | | | gccuauuuu | |
| V87 | S2 | G:U to | guuucaaucaaccgauagaaauauc | 401 | GCUCGUGGGUUUGUG | 497 |
| | | G:C/A:U | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuucc | | aaagagcaucggucugaaggau | |
| | | | ccgguaagaccucggaagcaagucc | | gcacuccgggauagggcaguccc | |
| | | | uucagcaagucgaaagacacgaug | | ggcucuugcuuuuccccgua | |
| | | | Cgagccuauuuu | | agaccucggaagcaaguccuuca | |
| | | | | | gcaagucgaaagacacgaugCg | |
| | | | | | agccuauuuu | |
| V88 | S2 | G:U to | guuucaaucaaccgauagaaauauc | 402 | GCUCGUGGGUUUGUG | 498 |
| | | G:C/A:U | gguccgguugaaaagaCcaucggu | | GUUGCCguuucaaucaaccg | |
| | | | cugaaggaugcacuccgggauagg | | auagaaauaucgguccgguuga | |
| | | | gcagucccggcucuugcuguuucc | | aaagaCcaucggucugaaggau | |
| | | | ccgguaagaccucggaagcaagucc | | gcacuccgggauagggcaguccc | |
| | | | uucagcaagucgaaagacacgaug | | ggcucuugcuguuuccccggua | |
| | | | Ggagccuauuuu | | agaccucggaagcaaguccuuca | |
| | | | | | gcaagucgaaagacacgaugGg | |
| | | | | | agccuauuuu | |
| V89 | S3 | G:U to | guuucaaucaaccgauagaaauauc | 403 | GCUCGUGGGUUUGUG | 499 |
| | | G:C/A:U | gguccgguugaaaagagcaucggu | | GUUGCCguuucaaucaaccg | |
| | | | cugaaggaCgcacuccgggauagg | | auagaaauaucgguccgguuga | |
| | | | gcagucccggcucuugcuguuucc | | aaagagcaucggucugaaggaC | |
| | | | ccgguaagaccucggaagcaagucc | | gcacuccgggauagggcaguccc | |
| | | | uucagcaagucgaaagacacgaug | | ggcucuugcuguuuccccggua | |
| | | | ugagccuauuuu | | agaccucggaagcaaguccuuca | |
| | | | | | gcaagucgaaagacacgauguga | |
| | | | | | gccuauuuu | |
| V90 | S3 | G:U to | guuucaaucaaccgauagaaauauc | 404 | GCUCGUGGGUUUGUG | 500 |
| | | G:C/A:U | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuucc | | aaagagcaucggucugaaggau | |
| | | | ccgguaagaccucggaagcaaAuc | | gcacuccgggauagggcaguccc | |
| | | | cuucagcaagucgaaagacacgaug | | ggcucuugcuguuuccccggua | |
| | | | ugagua | | agaccucggaagcaaAuccuuc | |
| | | | | | agcaagucgaaagacacgaugug | |
| | | | | | agccuauuuu | |
| V91 | S3 | G:U to | guuucaaucaaccgauagaaauauc | 405 | GCUCGUGGGUUUGUG | 501 |
| | | G:C/A:U | gguccgguugaaaagagcaucggu | | GUUGCCguuucaaucaaccg | |
| | | | cugaaggaGgcacuccgggauagg | | auagaaauaucgguccgguuga | |
| | | | gcagucccggcucuugcuguuucc | | aaagagcaucggucugaaggaG | |
| | | | ccgguaagaccucggaagcaaCuc | | gcacuccgggauagggcaguccc | |
| | | | cuucagcaagucgaaagacacgaug | | ggcucuugcuguuuccccggua | |
| | | | ugagua | | agaccucggaagcaaCuccuuca | |
| | | | | | gcaagucgaaagacacgauguga | |
| | | | | | gccuauuuu | |
| V92 | S4 | G:U to | guuucaaucaaccgauagaaauauc | 406 | GCUCGUGGGUUUGUG | 502 |
| | | G:C/A:U | gguccgguugaaaagagcaucggu | | GUUGCCguuucaaucaaccg | |
| | | | cugaaggaugcacuccgggaCagg | | auagaaauaucgguccgguuga | |
| | | | gcagucccggcucuugcuguuucc | | aaagagcaucggucugaaggau | |
| | | | ccgguaagaccucggaagcaagucc | | gcacuccgggaCagggcagucc | |
| | | | uucagcaagucgaaagacacgaug | | cggcucuugcuguuuccccggu | |
| | | | ugagccuauuuu | | aagaccucggaagcaaguccuuc | |
| | | | | | agcaagucgaaagacacgaugug | |
| | | | | | agccuauuuu | |
| V93 | S4 | G:U to | guuucaaucaaccgauagaaauauc | 407 | GCUCGUGGGUUUGUG | 503 |
| | | G:C/A:U | gguccgguugaaaagagcaucggu | | GUUGCCguuucaaucaaccg | |
| | | | cugaaggaugcacuccgggauagg | | auagaaauaucgguccgguuga | |
| | | | gcaAucccggcucuugcuguuucc | | aaagagcaucggucugaaggau | |
| | | | ccgguaagaccucggaagcaagucc | | gcacuccgggauagggcaAucc | |
| | | | uucagcaagucgaaagacacgaug | | cggcucuugcuguuuccccggu | |
| | | | ugagccuauuuu | | aagaccucggaagcaaguccuuc | |
| | | | | | agcaagucgaaagacacgaugug | |
| | | | | | agccuauuuu | |
| V94 | S4 | G:U to | guuucaaucaaccgauagaaauauc | 408 | GCUCGUGGGUUUGUG | 504 |
| | | G:C/A:U | gguccgguugaaaagagcaucggu | | GUUGCCguuucaaucaaccg | |
| | | | cugaaggaugcacuccgggaGagg | | auagaaauaucgguccgguuga | |
| | | | gcaCucccggcucuugcuguuucc | | aaagagcaucggucugaaggau | |
| | | | ccgguaagaccucggaagcaagucc | | gcacuccgggaGagggcaCucc | |
| | | | uucagcaagucgaaagacacgaug | | cggcucuugcuguuuccccggu | |
| | | | ugagccuauuuu | | aagaccucggaagcaaguccuuc | |
| | | | | | agcaagucgaaagacacgaugug | |
| | | | | | agccuauuuu | |
| WT | — | — | guuucaaucaaccgauagaaauauc | 409 | GCUCGUGGGUUUGUG | 505 |
| | | | gguccgguugaaaagagcaucggu | | GUUGCCguuucaaucaaccg | |
| | | | cugaaggaugcacuccgggauagg | | auagaaauaucgguccgguuga | |
| | | | gcagucccggcucuugcuguuucc | | aaagagcaucggucugaaggau | |
| | | | ccgguaagaccucggaagcaagucc | | gcacuccgggauagggcaguccc | |
| | | | uucagcaagucgaaagacacgaug | | ggcucuugcuguuuccccggua | |
| | | | ugagccuauuuu | | agaccucggaagcaaguccuuca | |
| | | | | | gcaagucgaaagacacgauguga | |
| | | | | | gccuauuuu | |
| NC | — | — | — | | — | |
| Combo1 | — | — | Guuucaaucaaccgagaaaucggu | 410 | GCUCGUGGGUUUGUG | 506 |
| (V2 + | | | ccgguugaaaagagcaucggcuga | | GUUGCCguuucaaucaaccg | |
| 31 + | | | aggaugcacuccgggauagggcag | | agaaaucgguccgguugaaaaga | |
| 38 + | | | ucccggcucuugcuaaguccuuca | | gcaucggcugaaggaugcacucc | |
| 44 + 70) | | | gcUUAcgaugugagccuuuuu | | gggauagggcagucccggcucu | |
| | | | | | ugcuaaguccuucagcUUAcg | |
| | | | | | augugagccuuuuu | |

TABLE 5
The editing efficiency of IID-25 with each sgRNA variant was
determined by percentage of indel formation from Table 4.

| | | Length of | | | | | | |
|---|---|---|---|---|---|---|---|---|
| | | sgRNA | | | | | | |
| | Length of | with 21-nt | | | | | | Relative |
| | scaffold | spacer | | | | | | activity |
| Variants | (nt) | (nt) | Rep1 | Rep2 | Rep3 | Average | STDEV | to WT |
| V1 | 156 | 177 | | 2.46 | 2.06 | 2.26 | 0.28 | 0.74 |
| V2 | 154 | 175 | 3.68 | 3.35 | 2.46 | 3.16 | 0.63 | 1.03 |
| V3 | 152 | 173 | 0.65 | 0.29 | 0.52 | 0.49 | 0.18 | 0.16 |
| V4 | 150 | 171 | 1.44 | 1.56 | 1.26 | 1.42 | 0.15 | 0.46 |
| V5 | 148 | 169 | 1.77 | 1.4 | 1.07 | 1.41 | 0.35 | 0.46 |
| V6 | 146 | 167 | 0.29 | 0.39 | 0.46 | 0.38 | 0.09 | 0.12 |
| V7 | 142 | 163 | 0.01 | 0.06 | 0.01 | 0.03 | 0.03 | 0.01 |
| V8 | 141 | 162 | — | — | — | — | — | — |
| V9 | 139 | 160 | 0.03 | 0.09 | 0.02 | 0.05 | 0.04 | 0.02 |
| V10 | 137 | 158 | 0.02 | 0.04 | 0.02 | 0.03 | 0.01 | 0.01 |
| V11 | 135 | 156 | 0.06 | 0.04 | 0.04 | 0.05 | 0.01 | 0.02 |
| V12 | 154 | 175 | 0.08 | 0.04 | 0.03 | 0.05 | 0.03 | 0.02 |
| V13 | 153 | 174 | 0.03 | 0.04 | 0.08 | 0.05 | 0.03 | 0.02 |
| V14 | 155 | 176 | 0.1 | 0.11 | 0.08 | 0.1 | 0.02 | 0.03 |
| V15 | 156 | 177 | 0.38 | 0.58 | 0.28 | 0.41 | 0.15 | 0.13 |
| V16 | 152 | 173 | 0.03 | 0.1 | 0.04 | 0.06 | 0.04 | 0.02 |
| V17 | 151 | 172 | 0.11 | 0.02 | 0.04 | 0.06 | 0.05 | 0.02 |
| V18 | 153 | 174 | 0.06 | 0.08 | 0.1 | 0.08 | 0.02 | 0.03 |
| V19 | 154 | 175 | 0.03 | 0.11 | 0.07 | 0.07 | 0.04 | 0.02 |
| V20 | 157 | 178 | 0.03 | 0.03 | 0.1 | 0.05 | 0.04 | 0.02 |
| V21 | 158 | 179 | 0.21 | 0.22 | 0.21 | 0.21 | 0.01 | 0.07 |
| V22 | 158 | 179 | 3.05 | 2.3 | 2.3 | 2.55 | 0.43 | 0.83 |
| V23 | 158 | 179 | 2.98 | 2.57 | 2.62 | 2.72 | 0.22 | 0.89 |
| V24 | 157 | 178 | 0.01 | 0.05 | 0.04 | 0.03 | 0.02 | 0.01 |
| V25 | 157 | 178 | 0.06 | 0.02 | 0 | 0.03 | 0.03 | 0.01 |
| V26 | 156 | 177 | 0.01 | 0.04 | 0.02 | 0.02 | 0.02 | 0.01 |
| V27 | 157 | 178 | 3.05 | 3.07 | 2.32 | 2.81 | 0.43 | 0.92 |
| V28 | 156 | 177 | 3.18 | 4.15 | 2.91 | 3.41 | 0.65 | 1.11 |
| V29 | 155 | 176 | 3.96 | 4.35 | 3.1 | 3.8 | 0.64 | 1.24 |
| V30 | 154 | 175 | 3.26 | 3.46 | 3.4 | 3.37 | 0.1 | 1.1 |
| V31 | 153 | 174 | 3.87 | 3.53 | 3.68 | 3.69 | 0.17 | 1.2 |
| V32 | 152 | 173 | 3.32 | 2.42 | 1.86 | 2.53 | 0.74 | 0.82 |
| V33 | 151 | 172 | 1.72 | 0.93 | | 1.33 | 0.56 | 0.43 |
| V34 | 150 | 171 | 0.03 | 0.05 | | 0.04 | 0.01 | 0.01 |
| V35 | 149 | 170 | 0.04 | 0.02 | | 0.03 | 0.01 | 0.01 |
| V36 | 148 | 169 | 0.03 | 0.02 | | 0.03 | 0.01 | 0.01 |
| V37 | 147 | 168 | 0.05 | 0 | | 0.03 | 0.04 | 0.01 |
| V38 | 157 | 178 | 6.53 | 5.34 | | 5.94 | 0.84 | 1.93 |
| V39 | 156 | 177 | 2.45 | 1.93 | | 2.19 | 0.37 | 0.71 |
| V40 | 154 | 175 | 2.04 | 1.29 | | 1.67 | 0.53 | 0.54 |
| V41 | 152 | 173 | 2.71 | 2.36 | 1.79 | 2.29 | 0.46 | 0.75 |
| V42 | 151 | 172 | 2.89 | 2.76 | 2.34 | 2.66 | 0.29 | 0.87 |
| V43 | 145 | 166 | 0.76 | 0.82 | 0.68 | 0.75 | 0.07 | 0.24 |
| V44 | 147 | 168 | 3.58 | 2.15 | 3.24 | 2.99 | 0.75 | 0.97 |
| V45 | 148 | 169 | 2.54 | 2.33 | 2.52 | 2.46 | 0.12 | 0.8 |
| V46 | 149 | 170 | 2.4 | 1.86 | 1.4 | 1.89 | 0.5 | 0.62 |
| V47 | 145 | 166 | 0.06 | 0.13 | 0.07 | 0.09 | 0.04 | 0.03 |
| V48 | 144 | 165 | 0.08 | 0.12 | 0.17 | 0.12 | 0.05 | 0.04 |
| V49 | 156 | 177 | 0.01 | 0.02 | 0.12 | 0.05 | 0.06 | 0.02 |
| V50 | 154 | 175 | 0.08 | 0.03 | 0.06 | 0.06 | 0.03 | 0.02 |
| V51 | 152 | 173 | 0.01 | 0.02 | 0.03 | 0.02 | 0.01 | 0.01 |
| V52 | 150 | 171 | 0.01 | 0.01 | 0.02 | 0.01 | 0.01 | 0 |
| V53 | 148 | 169 | 0.03 | 0.02 | 0.02 | 0.02 | 0.01 | 0.01 |
| V54 | 146 | 167 | 0.02 | 0.04 | 0.05 | 0.04 | 0.02 | 0.01 |
| V55 | 144 | 165 | 0.18 | 0.06 | 0.06 | 0.1 | 0.07 | 0.03 |
| V56 | 138 | 159 | 0.02 | 0.04 | 0.1 | 0.05 | 0.04 | 0.02 |
| V57 | 136 | 157 | 0.08 | 0.02 | 0.05 | 0.05 | 0.03 | 0.02 |
| V58 | 134 | 155 | 0.01 | 0.04 | 0.02 | 0.02 | 0.02 | 0.01 |
| V59 | 128 | 149 | 0.1 | 0.02 | 0.16 | 0.09 | 0.07 | 0.03 |
| V60 | 156 | 177 | 3.03 | 2.08 | 2.11 | 2.41 | 0.54 | 0.79 |
| V61 | 154 | 175 | 1.72 | 1.31 | 1.61 | 1.55 | 0.21 | 0.5 |
| V62 | 152 | 173 | 2.76 | 2.62 | 2.85 | 2.74 | 0.12 | 0.89 |
| V63 | 148 | 169 | 3.66 | 4.02 | 2.56 | 3.41 | 0.76 | 1.11 |
| V64 | 146 | 167 | 1.73 | 1.64 | 1.54 | 1.64 | 0.1 | 0.53 |
| V65 | 144 | 165 | 0.23 | 0.3 | 0.29 | 0.27 | 0.04 | 0.09 |
| V66 | 142 | 163 | 0.63 | 0.4 | 0.61 | 0.55 | 0.13 | 0.18 |
| V67 | 140 | 161 | 0.28 | 0.3 | 0.1 | 0.23 | 0.11 | 0.07 |
| V68 | 138 | 159 | 0.29 | 0.32 | 0.3 | 0.3 | 0.02 | 0.1 |
| V69 | 136 | 157 | 3.21 | 3.69 | 3.66 | 3.52 | 0.27 | 1.15 |
| V70 | 133 | 154 | 5.27 | 4.43 | 3.95 | 4.55 | 0.67 | 1.48 |
| V71 | 158 | 179 | 3.43 | 3.63 | 2.74 | 3.27 | 0.47 | 1.07 |
| V72 | 158 | 179 | 2.86 | 2.33 | 2.55 | 2.58 | 0.27 | 0.84 |
| V73 | 158 | 179 | | 3.03 | 1.91 | 2.47 | 0.79 | 0.8 |
| V74 | 158 | 179 | | 2.61 | 2.08 | 2.35 | 0.37 | 0.77 |
| V75 | 158 | 179 | | 3.23 | 2.19 | 2.71 | 0.74 | 0.88 |
| V76 | 158 | 179 | | 1.92 | 1.24 | 1.58 | 0.48 | 0.51 |
| V77 | 155 | 176 | | 1.07 | 0.67 | 0.87 | 0.28 | 0.28 |
| V78 | 154 | 175 | | 2.63 | 1.99 | 2.31 | 0.45 | 0.75 |
| V79 | 159 | 180 | | 3.64 | 3.4 | 3.52 | 0.17 | 1.15 |
| V80 | 157 | 178 | | 3.03 | 3.44 | 3.24 | 0.29 | 1.06 |
| V81 | 155 | 176 | 0.92 | 0.94 | 0.86 | 0.91 | 0.04 | 0.3 |
| V82 | 153 | 174 | 2.28 | 3.07 | 1.78 | 2.38 | 0.65 | 0.78 |
| V83 | 158 | 179 | 2.9 | 2.29 | 2.14 | 2.44 | 0.4 | 0.79 |
| V84 | 158 | 179 | 1.04 | 1 | 1.27 | 1.1 | 0.15 | 0.36 |
| V85 | 158 | 179 | 0.04 | 0.04 | 0.02 | 0.03 | 0.01 | 0.01 |
| V86 | 158 | 179 | 2.36 | 2.3 | 1.76 | 2.14 | 0.33 | 0.7 |
| V87 | 158 | 179 | 2.64 | 2.67 | 2.15 | 2.49 | 0.29 | 0.81 |
| V88 | 158 | 179 | 2.16 | 2.61 | 2.08 | 2.28 | 0.29 | 0.74 |
| V89 | 158 | 179 | — | — | — | — | — | — |
| V90 | 158 | 179 | 2.42 | 2.39 | 2.28 | 2.36 | 0.07 | 0.77 |
| V91 | 158 | 179 | 1.08 | 1.59 | 1.48 | 1.38 | 0.27 | 0.45 |
| V92 | 158 | 179 | 1.86 | 2.3 | 1.55 | 1.9 | 0.38 | 0.62 |
| V93 | 158 | 179 | 1.03 | 1.06 | 1.2 | 1.1 | 0.09 | 0.36 |
| V94 | 158 | 179 | 2.38 | 2.24 | 1.67 | 2.1 | 0.38 | 0.68 |
| WT | 158 | 179 | 3.25 | | 2.89 | 3.07 | 0.25 | 1 |
| NC | — | — | 0.09 | 0 | 0.01 | 0.03 | 0.05 | 0.01 |
| Combo1 | 117 | 138 | — | — | — | — | — | — |
| (V2 + 31 + 38 + 44 + 70) | | | | | | | | |

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as embodied should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

### Example 5: Cas9IID-86 and IID-41 Mutants

Table 6 below shows exemplary mutations in WT Cas9-IID-86 (SEQ ID NO: 12) or WT Cas9-IID-41 (SEQ ID NO: 27).

TABLE 6
Cas9 IID-86 and IID-41 mutants

| IID-86 mutations | IID-41 mutations |
|---|---|
| (SEQ ID NO: 12) | (SEQ ID NO: 27) |
| S11K | V13K |
| W40R | C21R |
| H63R | T36R |
| Q67R or Q67K | E40R |
| E70R or E70K | V41R |
| Q71R or Q71K | S64R |
| S71R | N67R |
| S84K | Q71K |
| N90R | A72K |
| E109K | A85K |
| T127K | H88R |
| S131R | E104K |
| Q138K | E107K |
| S144K | N108K |
| T148K or T148R | G109K |
| Q153K or Q153R | Q110K |
| L158K or L158R | A111K |
| Q162K | L139K |
| S185R | P142K |
| G190R | H154R |
| A204K | L213R |
| C212R or C212K | H214R |
| E221R | D216K |
| E224R | A219R |
| N239K | T223R |
| E257K | E226R |
| E277R | D240K |
| T278R | D264K |
| S287K | V268R |
| Q315K | S270R |
| S320R | E271R |
| L363Ins(NKKKSRR, | G292K |
| SEQ ID NO: 507) | |
| T369K | C297R |
| I370R | A308K |
| Q376K | L356Ins(NKKKSRR, |
| | SEQ ID NO: 507) |
| M387R | Q362K |
| E391K | D368R |
| V393R | Q392K |
| E399K | A443R |
| Q410K | A465K |
| V418R | F468K |
| H424R | I470K |
| A425R | Q498R |
| S444R | N505K |
| D449R | Q530K |
| T458R | S535K |
| D469R | Q537K |
| S476K | A539R |
| T477K | E564K |
| Q495R | L568K |
| Q541K | D581K |
| A542K | T586R |
| A544K | L591R |
| A568R | V602R |
| A571K | A604K |
| Y593K | T606R |
| L598R | V607K |
| P601R | A608K |
| A609R | P611K |
| D610R | P614K |
| T611K | E622K |
| D621K | G624R |
| D629K | E632K |
| P631R | G654K |
| T639K | S657K |
| S652K | N666K |
| M661K | N668K |
| E672K | N688K |
| G675K | T689R |
| M676K | S690K |
| P696R | G691K |
| S701K | I695K |
| I738K | T698K |
| A745K | Q706K |
| H746K | Q709R |
| N751R | D738K |
| A757R | N744R |
| S760K | A750R |
| M781R | D757K |
| Q798K | S780R |
| G801K | P813K |
| D828K | L814K |
| T844K or T844R | S823R |
| I848K | A843K |
| S849R | P845K |
| P854K | A851K |
| D860K | P858K |
| V862R | D863K |
| G872K | A867K |
| N905K | E875K |
| S914K | T905K |
| E916K | Q907K |
| S937R | N911K |
| V943K | F913R |
| | V918K |
| | A928R |
| | V933K |
| | P790R |

### Example 6: Testing Synthetic IID25 TTR Guides in AML-12 Cells

Several different scaffolds with different spacers were tested to see which has the best effect on reducing transthyretin (TTR) expression levels in AML-12 cells. AML12 (CRL-2254, alpha mouse liver 12) cells are hepatocytes isolated from the normal liver of a 3-month-old mouse. To demonstrate targeting and cleavage activity in AML-12 cells, plasmids expressing the Cas9 IID protein sequence (IID25 SEQ ID NO: 1 with Q913K) and the engineered single guide RNA sequences (e.g., V70, V18, V131; see e.g., Tables 7A-7D) were transfected into AML-12 cells using LIPOFECTAMINE MESSENGERMAX. Cell media at 72 hours after cotransfection was saved prior to harvest so that an ELISA on the TTR protein could be done to correlate indels with knockdown in protein expression. Genomic DNA was then harvested 72 hrs after cotransfection using QUICK EXTRACT. The target sequence in the genomic DNA was amplified (see e.g., primers in Table 8) and sequenced with NGS using an ILLUMINA MISEQ instrument. V118 exhibited enhanced activity (see e.g., FIG. 1-2).

TABLE 7A
Exemplary Guide Sequences: SI# indicates the corresponding SEQ ID NO.

| | Target | Target | Target | Scaffold | | Scaffold |
|---|---|---|---|---|---|---|
| RNA ID | ID | Seq | SI# | ID | Scaffold Seq | SI# |
| IID25_S | EMX1- | ctccaat | 509 | v70 | gtttcaatcaaccgatagaaatatcggtccggttgaa | 524 |
| yn_gRN | 2 | gactag | | | aagagcatcggtctgaaggatgcactccgggatag | |
| A001 | | ggtggg | | | ggcagtcccggctcttgctaagtccttcagcaagtc | |
| | | C | | | gaaagacacgatgtgagcctatttt | |
| IID25_S | EMX1- | atgggct | 510 | v70 | gtttcaatcaaccgatagaaatatcggtccggttgaa | 524 |
| yn_gRN | 4 | ggctga | | | aagagcatcggtctgaaggatgcactccgggatag | |
| A002 | | ggcctg | | | ggcagtcccggctcttgctaagtccttcagcaagtc | |
| | | g | | | gaaagacacgatgtgagcctatttt | |
| IID25_S | HPRT3 | agacaa | 511 | v70 | gtttcaatcaaccgatagaaatatcggtccggttgaa | 524 |
| yn_gRN | 807 | ggcactt | | | aagagcatcggtctgaaggatgcactccgggatag | |
| A003 | | ggttgat | | | ggcagtcccggctcttgctaagtccttcagcaagtc | |
| | | | | | gaaagacacgatgtgagcctatttt | |
| IID25_S | VEGF | cccacac | 523 | v70 | gtttcaatcaaccgatagaaatatcggtccggttgaa | 524 |
| yn_gRN | A1 | gcacac | | | aagagcatcggtctgaaggatgcactccgggatag | |
| A004 | | actcact | | | ggcagtcccggctcttgctaagtccttcagcaagtc | |
| | | | | | gaaagacacgatgtgagcctatttt | |
| IID25_S | IID25 | GACT | 515 | v70 | gtttcaatcaaccgatagaaatatcggtccggttgaa | 524 |
| yn_gRN | PT_5_ | TGGG | | | aagagcatcggtctgaaggatgcactccgggatag | |
| A005 | TTLL1 | TGAG | | | ggcagtcccggctcttgctaagtccttcagcaagtc | |
| | 0 | CGAT | | | gaaagacacgatgtgagcctatttt | |
| | | GGAC | | | | |
| IID25_S | IID25 | GGAC | 512 | v70 | gtttcaatcaaccgatagaaatatcggtccggttgaa | 524 |
| yn_gRN | PT_10_ | CTCC | | | aagagcatcggtctgaaggatgcactccgggatag | |
| A006 | CIQT | AGGT | | | ggcagtcccggctcttgctaagtccttcagcaagtc | |
| | NF12 | GCAG | | | gaaagacacgatgtgagcctatttt | |
| | | AAGT | | | | |
| IID25_S | IID25 | CTGT | 513 | v70 | gtttcaatcaaccgatagaaatatcggtccggttgaa | 524 |
| yn_gRN | PT_15_ | GAAG | | | aagagcatcggtctgaaggatgcactccgggatag | |
| A007 | CPTP | AGCC | | | ggcagtcccggctcttgctaagtccttcagcaagtc | |
| | | AGTG | | | gaaagacacgatgtgagcctatttt | |
| | | GCCA | | | | |
| IID25_S | IID25 | GGAC | 514 | v70 | gtttcaatcaaccgatagaaatatcggtccggttgaa | 524 |
| yn_gRN | PT_22 | AAAG | | | aagagcatcggtctgaaggatgcactccgggatag | |
| A008 | VWA | GTCC | | | ggcagtcccggctcttgctaagtccttcagcaagtc | |
| | 1 | TCAA | | | gaaagacacgatgtgagcctatttt | |
| | | AGAC | | | | |
| IID25_S | IID25 | GACT | 515 | V117 | gCTTcaaTcaaccgaTagaaaTaTcggTcc | 525 |
| yn_gRN | PT_5_ | TGGG | | | ggTTgaaGagagcaTcggTcTgaaggaTgc | |
| A009 | TTLL10 | TGAG | | | acTccgggaTagggcagTcccggcTcTTgc | |
| | | CGAT | | | TaagTccTTcagcaagTcgaaagacacgaTg | |
| | | GGAC | | | TgagccTaTTTT | |
| IID25_S | IID25 | GACT | 515 | V118 | gTCTcaaTcaaccgaTagaaaTaTcggTcc | 526 |
| yn_gRN | PT_5_ | TGGG | | | ggTTgaGaagagcaTcggTcTgaaggaTgc | |
| A010 | TTLL10 | TGAG | | | acTccgggaTagggcagTcccggcTcTTgc | |
| | | CGAT | | | TaagTccTTcagcaagTcgaaagacacgaTg | |
| | | GGAC | | | TgagccTaTTTT | |
| IID25_S | IID25 | GACT | 515 | V123 | gTTTcGaTcaaccgaTagaaaTaTcggTcc | 527 |
| yn_gRN | PT_5_ | TGGG | | | ggTCgaaaagagcaTcggTcTgaaggaTgc | |
| A011 | TTLL10 | TGAG | | | acTccgggaTagggcagTcccggcTcTTgc | |
| | | CGAT | | | TaagTccTTcagcaagTcgaaagacacgaTg | |
| | | GGAC | | | TgagccTaTTTT | |
| IID25_S | IID25 | GACT | 515 | V126 | gTTTcaaTcaGccgaTagaaaTaTcggCcc | 528 |
| yn_gRN | PT_5_ | TGGG | | | ggTTgaaaagagcaTcggTcTgaaggaTgca | |
| A012 | TTLL10 | TGAG | | | cTccgggaTagggcagTcccggcTcTTgcT | |
| | | CGAT | | | aagTccTTcagcaagTcgaaagacacgaTgT | |
| | | GGAC | | | gagccTaTTTT | |
| IID25_S | IID25 | GACT | 515 | V127 | gTTTcaaTcaCccgaTagaaaTaTcggGcc | 529 |
| yn_gRN | PT_5_ | TGGG | | | ggTTgaaaagagcaTcggTcTgaaggaTgca | |
| A013 | TTLL10 | TGAG | | | cTccgggaTagggcagTcccggcTcTTgcT | |
| | | CGAT | | | aagTccTTcagcaagTcgaaagacacgaTgT | |
| | | GGAC | | | gagccTaTTTT | |
| IID25_S | IID25 | GACT | 515 | V128 | gTTTcaaTcaaccgaTgaaaaTcggTccgg | 530 |
| yn_gRN | PT_5_ | TGGG | | | TTgaaaagagcaTcggTcTgaaggaTgcac | |
| A014 | TTLL10 | TGAG | | | TccgggaTagggcagTcccggcTcTTgcTa | |
| | | CGAT | | | agTccTTcagcaagTcgaaagacacgaTgTg | |
| | | GGAC | | | agccTaTTTT | |
| IID25_S | IID25 | GACT | 515 | V129 | gTTTcaaTcaaccgagaaaTcggTccggTT | 531 |
| yn_gRN | PT_5_ | TGGG | | | gaaaagagcaTcggTcTgaaggaTgcacTcc | |
| A015 | TTLL10 | TGAG | | | gggaTagggcagTcccggcTcTTgcTaagT | |
| | | CGAT | | | ccTTcagcaagTcgaaagacacgaTgTgagc | |
| | | GGAC | | | cTaTTTT | |
| IID25_S | IID25 | GACT | 515 | V130 | gTTTcaaTcaaccgaTagaaaTaTcggTcc | 532 |
| yn_gRN | PT_5_ | TGGG | | | ggTTgaaaagagcaTcggcTgaaggaTgcac | |
| A016 | TTLL10 | TGAG | | | TccgggaTagggcagTcccggcTcTTgcTa | |
| | | CGAT | | | agTccTTcagcaagTcgaaagacacgaTgTg | |
| | | GGAC | | | agccTaTTTT | |
| IID25_S | IID25 | GACT | 515 | V134 | gTTTcaaTcaaccgaTagaaaTaTcggTcc | 533 |
| yn_gRN | PT_5_ | TGGG | | | ggTTgaaaagagcaTcggTcTgaaggaTgca | |
| A017 | TTLL10 | TGAG | | | cTccgggaTagggcagTcccggcTcTTgcT | |
| | | CGAT | | | aagTccTTcagcaagcgaaagcacgaTgTga | |
| | | GGAC | | | gccTaTTTT | |
| IID25_S | IID25 | GGAC | 512 | V117 | gCTTcaaTcaaccgaTagaaaTaTcggTcc | 525 |
| yn_gRN | PT_10_ | CTCC | | | ggTTgaaGagagcaTcggTcTgaaggaTgc | |
| A018 | CIQT | AGGT | | | acTccgggaTagggcagTcccggcTcTTgc | |
| | NF12 | GCAG | | | TaagTccTTcagcaagTcgaaagacacgaTg | |
| | | AAGT | | | TgagccTaTTTT | |
| IID25_S | IID25 | GGAC | 512 | V118 | gTCTcaaTcaaccgaTagaaaTaTcggTcc | 526 |
| yn_gRN | PT_10_ | CTCC | | | ggTTgaGaagagcaTcggTcTgaaggaTgc | |
| A019 | CIQT | AGGT | | | acTccgggaTagggcagTcccggcTcTTgc | |
| | NF12 | GCAG | | | TaagTccTTcagcaagTcgaaagacacgaTg | |
| | | AAGT | | | TgagccTaTTTT | |
| IID25_S | IID25 | GGAC | 512 | V123 | gTTTcGaTcaaccgaTagaaaTaTcggTcc | 527 |
| yn_gRN | PT_10_ | CTCC | | | ggTCgaaaagagcaTcggTcTgaaggaTgc | |
| A020 | CIQT | AGGT | | | acTccgggaTagggcagTcccggcTcTTgc | |
| | NF12 | GCAG | | | TaagTccTTcagcaagTcgaaagacacgaTg | |
| | | AAGT | | | TgagccTaTTTT | |
| IID25_S | IID25 | GGAC | 512 | V126 | gTTTcaaTcaGccgaTagaaaTaTcggCcc | 528 |
| yn_gRN | PT_10_ | CTCC | | | ggTTgaaaagagcaTcggTcTgaaggaTgca | |
| A021 | CIQT | AGGT | | | cTccgggaTagggcagTcccggcTcTTgcT | |
| | NF12 | GCAG | | | aagTccTTcagcaagTcgaaagacacgaTgT | |
| | | AAGT | | | gagccTaTTTT | |
| IID25_S | IID25 | GGAC | 512 | V127 | gTTTcaaTcaCccgaTagaaaTaTcggGcc | 529 |
| yn_gRN | PT_10_ | CTCC | | | ggTTgaaaagagcaTcggTcTgaaggaTgca | |
| A022 | CIQT | AGGT | | | cTccgggaTagggcagTcccggcTcTTgcT | |
| | NF12 | GCAG | | | aagTccTTcagcaagTcgaaagacacgaTgT | |
| | | AAGT | | | gagccTaTTTT | |
| IID25_S | IID25 | GGAC | 512 | V128 | gTTTcaaTcaaccgaTgaaaaTcggTccgg | 530 |
| yn_gRN | PT_10_ | CTCC | | | TTgaaaagagcaTcggTcTgaaggaTgcac | |
| A023 | CIQT | AGGT | | | TccgggaTagggcagTcccggcTcTTgcTa | |
| | NF12 | GCAG | | | agTccTTcagcaagTcgaaagacacgaTgTg | |
| | | AAGT | | | agccTaTTTT | |
| IID25_S | IID25 | GGAC | 512 | V129 | gTTTcaaTcaaccgagaaaTcggTccggTT | 531 |
| yn_gRN | PT_10_ | CTCC | | | gaaaagagcaTcggTcTgaaggaTgcacTcc | |
| A024 | CIQT | AGGT | | | gggaTagggcagTcccggcTcTTgcTaagT | |
| | NF12 | GCAG | | | ccTTcagcaagTcgaaagacacgaTgTgagc | |
| | | AAGT | | | cTaTTTT | |
| IID25_S | IID25 | GGAC | 512 | V130 | gTTTcaaTcaaccgaTagaaaTaTcggTcc | 532 |
| yn_gRN | PT_10_ | CTCC | | | ggTTgaaaagagcaTcggcTgaaggaTgcac | |
| A025 | C1QT | AGGT | | | TccgggaTagggcagTcccggcTcTTgcTa | |
| | NF12 | GCAG | | | agTccTTcagcaagTcgaaagacacgaTgTg | |
| | | AAGT | | | agccTaTTTT | |
| IID25_S | IID25 | GGAC | 512 | V134 | gTTTcaaTcaaccgaTagaaaTaTcggTcc | 533 |
| yn_gRN | PT_10_ | CTCC | | | ggTTgaaaagagcaTcggTcTgaaggaTgca | |
| A026 | CIQT | AGGT | | | cTccgggaTagggcagTcccggcTcTTgcT | |
| | NF12 | GCAG | | | aagTccTTcagcaagcgaaagcacgaTgTga | |
| | | AAGT | | | gccTaTTTT | |
| IID25_S | IID25 | CTGT | 513 | V117 | gCTTcaaTcaaccgaTagaaaTaTcggTcc | 525 |
| yn_gRN | PT_15_ | GAAG | | | ggTTgaaGagagcaTcggTcTgaaggaTgc | |
| A027 | CPTP | AGCC | | | acTccgggaTagggcagTcccggcTcTTgc | |
| | | AGTG | | | TaagTccTTcagcaagTcgaaagacacgaTg | |
| | | GCCA | | | TgagccTaTTTT | |
| IID25_S | IID25 | CTGT | 513 | V118 | gTCTcaaTcaaccgaTagaaaTaTcggTcc | 526 |
| yn_gRN | PT_15_ | GAAG | | | ggTTgaGaagagcaTcggTcTgaaggaTgc | |
| A028 | CPTP | AGCC | | | acTccgggaTagggcagTcccggcTcTTgc | |
| | | AGTG | | | TaagTccTTcagcaagTcgaaagacacgaTg | |
| | | GCCA | | | TgagccTaTTTT | |
| IID25_S | IID25 | CTGT | 513 | V123 | gTTTcGaTcaaccgaTagaaaTaTcggTcc | 527 |
| yn_gRN | PT_15_ | GAAG | | | ggTCgaaaagagcaTcggTcTgaaggaTgc | |
| A029 | CPTP | AGCC | | | acTccgggaTagggcagTcccggcTcTTgc | |
| | | AGTG | | | TaagTccTTcagcaagTcgaaagacacgaTg | |
| | | GCCA | | | TgagccTaTTTT | |
| IID25_S | IID25 | CTGT | 513 | V126 | gTTTcaaTcaGccgaTagaaaTaTcggCcc | 528 |
| yn_gRN | PT_15_ | GAAG | | | ggTTgaaaagagcaTcggTcTgaaggaTgca | |
| A030 | CPTP | AGCC | | | cTccgggaTagggcagTcccggcTcTTgcT | |
| | | AGTG | | | aagTccTTcagcaagTcgaaagacacgaTgT | |
| | | GCCA | | | gagccTaTTTT | |
| IID25_S | IID25 | CTGT | 513 | V127 | gTTTcaaTcaCccgaTagaaaTaTcggGcc | 529 |
| yn_gRN | PT_15_ | GAAG | | | ggTTgaaaagagcaTcggTcTgaaggaTgca | |
| A031 | CPTP | AGCC | | | cTccgggaTagggcagTcccggcTcTTgcT | |
| | | AGTG | | | aagTccTTcagcaagTcgaaagacacgaTgT | |
| | | GCCA | | | gagccTaTTTT | |
| IID25_S | IID25 | CTGT | 513 | V128 | gTTTcaaTcaaccgaTgaaaaTcggTccgg | 530 |
| yn_gRN | PT_15_ | GAAG | | | TTgaaaagagcaTcggTcTgaaggaTgcac | |
| A032 | CPTP | AGCC | | | TccgggaTagggcagTcccggcTcTTgcTa | |
| | | AGTG | | | agTccTTcagcaagTcgaaagacacgaTgTg | |
| | | GCCA | | | agccTaTTTT | |
| IID25_S | IID25 | CTGT | 513 | V129 | gTTTcaaTcaaccgagaaaTcggTccggTT | 531 |
| yn_gRN | PT_15_ | GAAG | | | gaaaagagcaTcggTcTgaaggaTgcacTcc | |
| A033 | CPTP | AGCC | | | gggaTagggcagTcccggcTcTTgcTaagT | |
| | | AGTG | | | ccTTcagcaagTcgaaagacacgaTgTgagc | |
| | | GCCA | | | cTaTTTT | |
| IID25_S | IID25 | CTGT | 513 | V130 | gTTTcaaTcaaccgaTagaaaTaTcggTcc | 532 |
| yn_gRN | PT_15_ | GAAG | | | ggTTgaaaagagcaTcggcTgaaggaTgcac | |
| A034 | CPTP | AGCC | | | TccgggaTagggcagTcccggcTcTTgcTa | |
| | | AGTG | | | agTccTTcagcaagTcgaaagacacgaTgTg | |
| | | GCCA | | | agccTaTTTT | |
| IID25_S | IID25 | CTGT | 513 | V134 | gTTTcaaTcaaccgaTagaaaTaTcggTcc | 533 |
| yn_gRN | PT_15_ | GAAG | | | ggTTgaaaagagcaTcggTcTgaaggaTgca | |
| A035 | CPTP | AGCC | | | cTccgggaTagggcagTcccggcTcTTgcT | |
| | | AGTG | | | aagTccTTcagcaagcgaaagcacgaTgTga | |
| | | GCCA | | | gccTaTTTT | |
| IID25_S | IID25 | GACT | 515 | V149 | GCCTCAATCAACCGATAGAAAT | 534 |
| yn_gRN | PT_5_ | TGGG | | | ATCGGTCCGGTTGAGGAGAGCA | |
| A036 | TTLL1 | TGAG | | | TCGGTCTGAAGGATGCACTCCG | |
| | 0 | CGAT | | | GGATAGGGCAGTCCCGGCTCTT | |
| | | GGAC | | | GCTAAGTCCTTCAGCAAGTCGA | |
| | | | | | AAGACACGATGTGAGCCTATTT | |
| | | | | | T | |
| IID25_S | IID25 | GACT | 515 | V150 | GCCTCGATCAACCGATAGAAAT | 535 |
| yn_gRN | PT_5_ | TGGG | | | ATCGGTCCGGTCGAGGAGAGCA | |
| A037 | TTLL1 | TGAG | | | TCGGTCTGAAGGATGCACTCCG | |
| | 0 | CGAT | | | GGATAGGGCAGTCCCGGCTCTT | |
| | | GGAC | | | GCTAAGTCCTTCAGCAAGTCGA | |
| | | | | | AAGACACGATGTGAGCCTATTT | |
| | | | | | T | |
| IID25_S | IID25 | GACT | 515 | V151 | GTCTCAATCAACCGATAGAAAT | 536 |
| yn_gRN | PT_5_ | TGGG | | | ATCGGTCCGGTTGAGAAGAGCA | |
| A038 | TTLL1 | TGAG | | | TCGGCTGAAGGATGCACTCCGG | |
| | 0 | CGAT | | | GATAGGGCAGTCCCGGCTCTTG | |
| | | GGAC | | | CTAAGTCCTTCAGCAAGCGAAA | |
| | | | | | GCACGATGTGAGCCTATTTT | |
| IID25_S | IID25 | GACT | 515 | V152 | GTCTCAATCAACCGAGAAATCG | 537 |
| yn_gRN | PT_5_ | TGGG | | | GTCCGGTTGAAAAGAGCATCGG | |
| A039 | TTLL1 | TGAG | | | CTGAAGGATGCACTCCGGGATA | |
| | 0 | CGAT | | | GGGCAGTCCCGGCTCTTGCTAA | |
| | | GGAC | | | GTCCTTCAGCAAGCGAAAGCAC | |
| | | | | | GATGTGAGCCTATTTT | |
| IID25_S | IID25 | GGAC | 512 | V149 | GCCTCAATCAACCGATAGAAAT | 534 |
| yn_gRN | PT_10_ | CTCC | | | ATCGGTCCGGTTGAGGAGAGCA | |
| A040 | CIQT | AGGT | | | TCGGTCTGAAGGATGCACTCCG | |
| | NF12 | GCAG | | | GGATAGGGCAGTCCCGGCTCTT | |
| | | AAGT | | | GCTAAGTCCTTCAGCAAGTCGA | |
| | | | | | AAGACACGATGTGAGCCTATTT | |
| | | | | | T | |
| IID25_S | IID25 | GGAC | 512 | V150 | GCCTCGATCAACCGATAGAAAT | 535 |
| yn_gRN | PT_10_ | CTCC | | | ATCGGTCCGGTCGAGGAGAGCA | |
| A041 | CIQT | AGGT | | | TCGGTCTGAAGGATGCACTCCG | |
| | NF12 | GCAG | | | GGATAGGGCAGTCCCGGCTCTT | |
| | | AAGT | | | GCTAAGTCCTTCAGCAAGTCGA | |
| | | | | | AAGACACGATGTGAGCCTATTT | |
| | | | | | T | |
| IID25_S | IID25 | GGAC | 512 | V151 | GTCTCAATCAACCGATAGAAAT | 536 |
| yn_gRN | PT_10_ | CTCC | | | ATCGGTCCGGTTGAGAAGAGCA | |
| A042 | CIQT | AGGT | | | TCGGCTGAAGGATGCACTCCGG | |
| | NF12 | GCAG | | | GATAGGGCAGTCCCGGCTCTTG | |
| | | AAGT | | | CTAAGTCCTTCAGCAAGCGAAA | |
| | | | | | GCACGATGTGAGCCTATTTT | |
| IID25_S | IID25 | GGAC | 512 | V152 | GTCTCAATCAACCGAGAAATCG | 537 |
| yn_gRN | PT_10_ | CTCC | | | GTCCGGTTGAAAAGAGCATCGG | |
| A043 | C1QT | AGGT | | | CTGAAGGATGCACTCCGGGATA | |
| | NF12 | GCAG | | | GGGCAGTCCCGGCTCTTGCTAA | |
| | | AAGT | | | GTCCTTCAGCAAGCGAAAGCAC | |
| | | | | | GATGTGAGCCTATTTT | |
| IID25_S | IID25 | CTGT | 513 | V149 | GCCTCAATCAACCGATAGAAAT | 534 |
| yn_gRN | PT_15_ | GAAG | | | ATCGGTCCGGTTGAGGAGAGCA | |
| A044 | CPTP | AGCC | | | TCGGTCTGAAGGATGCACTCCG | |
| | | AGTG | | | GGATAGGGCAGTCCCGGCTCTT | |
| | | GCCA | | | GCTAAGTCCTTCAGCAAGTCGA | |
| | | | | | AAGACACGATGTGAGCCTATTT | |
| | | | | | T | |
| IID25_S | IID25 | CTGT | 513 | V150 | GCCTCGATCAACCGATAGAAAT | 535 |
| yn_gRN | PT_15_ | GAAG | | | ATCGGTCCGGTCGAGGAGAGCA | |
| A045 | CPTP | AGCC | | | TCGGTCTGAAGGATGCACTCCG | |
| | | AGTG | | | GGATAGGGCAGTCCCGGCTCTT | |
| | | GCCA | | | GCTAAGTCCTTCAGCAAGTCGA | |
| | | | | | AAGACACGATGTGAGCCTATTT | |
| | | | | | T | |
| IID25_S | IID25 | CTGT | 513 | V151 | GTCTCAATCAACCGATAGAAAT | 536 |
| yn_gRN | PT_15_ | GAAG | | | ATCGGTCCGGTTGAGAAGAGCA | |
| A046 | CPTP | AGCC | | | TCGGCTGAAGGATGCACTCCGG | |
| | | AGTG | | | GATAGGGCAGTCCCGGCTCTTG | |
| | | GCCA | | | CTAAGTCCTTCAGCAAGCGAAA | |
| | | | | | GCACGATGTGAGCCTATTTT | |
| IID25_S | IID25 | CTGT | 513 | V152 | GTCTCAATCAACCGAGAAATCG | 537 |
| yn_gRN | PT_15_ | GAAG | | | GTCCGGTTGAAAAGAGCATCGG | |
| A047 | CPTP | AGCC | | | CTGAAGGATGCACTCCGGGATA | |
| | | AGTG | | | GGGCAGTCCCGGCTCTTGCTAA | |
| | | GCCA | | | GTCCTTCAGCAAGCGAAAGCAC | |
| | | | | | GATGTGAGCCTATTTT | |
| IID25_S | TTR1 | AGCC | 517 | v70 | gtttcaatcaaccgatagaaatatcggtccggttgaa | 524 |
| yn_gRN | | ACGT | | | aagagcatcggtctgaaggatgcactccgggatag | |
| A048 | | CTAC | | | ggcagtcccggctcttgctaagtccttcagcaagtc | |
| | | AGCA | | | gaaagacacgatgtgagcctatttt | |
| | | GGGC | | | | |
| IID25_S | TTR6 | ACAG | 519 | v70 | gtttcaatcaaccgatagaaatatcggtccggttgaa | 524 |
| yn_gRN | | CAGG | | | aagagcatcggtctgaaggatgcactccgggatag | |
| A049 | | GCTG | | | ggcagtcccggctcttgctaagtccttcagcaagtc | |
| | | CCTC | | | gaaagacacgatgtgagcctatttt | |
| | | GGAC | | | | |
| IID25_S | TTR7 | GAGG | 521 | v70 | gtttcaatcaaccgatagaaatatcggtccggttgaa | 524 |
| yn_gRN | | GATC | | | aagagcatcggtctgaaggatgcactccgggatag | |
| A050 | | CTGG | | | ggcagtcccggctcttgctaagtccttcagcaagtc | |
| | | GAGC | | | gaaagacacgatgtgagcctatttt | |
| | | CCTT | | | | |
| IID25_S | TTR1 | AGCC | 517 | V118 | gTCTcaaTcaaccgaTagaaaTaTcggTcc | 526 |
| yn_gRN | | ACGT | | | ggTTgaGaagagcaTcggTcTgaaggaTgc | |
| A051 | | CTAC | | | acTccgggaTagggcagTcccggcTcTTgc | |
| | | AGCA | | | TaagTccTTcagcaagTcgaaagacacgaTg | |
| | | GGGC | | | TgagccTaTTTT | |
| IID25_S | TTR6 | ACAG | 519 | V118 | gTCTcaaTcaaccgaTagaaaTaTcggTcc | 526 |
| yn_gRN | | CAGG | | | ggTTgaGaagagcaTcggTcTgaaggaTgc | |
| A052 | | GCTG | | | acTccgggaTagggcagTcccggcTcTTgc | |
| | | CCTC | | | TaagTccTTcagcaagTcgaaagacacgaTg | |
| | | GGAC | | | TgagccTaTTTT | |
| IID25_S | TTR7 | GAGG | 521 | V118 | gTCTcaaTcaaccgaTagaaaTaTcggTcc | 526 |
| yn_gRN | | GATC | | | ggTTgaGaagagcaTcggTcTgaaggaTgc | |
| A053 | | CTGG | | | acTccgggaTagggcagTcccggcTcTTgc | |
| | | GAGC | | | TaagTccTTcagcaagTcgaaagacacgaTg | |
| | | CCTT | | | TgagccTaTTTT | |
| IID25_S | TTR1 | TTAC | 518 | v70 | gtttcaatcaaccgatagaaatatcggtccggttgaa | 524 |
| yn_gRN | + 4 | AGCC | | | aagagcatcggtctgaaggatgcactccgggatag | |
| A054 | | ACGT | | | ggcagtcccggctcttgctaagtccttcagcaagtc | |
| | | CTAC | | | gaaagacacgatgtgagcctatttt | |
| | | AGCA | | | | |
| | | GGGC | | | | |
| IID25_S | TTR6 | GTCT | 520 | v70 | gtttcaatcaaccgatagaaatatcggtccggttgaa | 524 |
| yn_gRN | + 4 | ACAG | | | aagagcatcggtctgaaggatgcactccgggatag | |
| A055 | | CAGG | | | ggcagtcccggctcttgctaagtccttcagcaagtc | |
| | | GCTG | | | gaaagacacgatgtgagcctatttt | |
| | | CCTC | | | | |
| | | GGAC | | | | |
| IID25_S | TTR7 | CTCT | 522 | v70 | gtttcaatcaaccgatagaaatatcggtccggttgaa | 524 |
| yn_gRN | + 4 | GAGG | | | aagagcatcggtctgaaggatgcactccgggatag | |
| A056 | | GATC | | | ggcagtcccggctcttgctaagtccttcagcaagtc | |
| | | CTGG | | | gaaagacacgatgtgagcctatttt | |
| | | GAGC | | | | |
| | | CCTT | | | | |
| IID25_S | TTR1 | TTAC | 518 | V118 | gTCTcaaTcaaccgaTagaaaTaTcggTcc | 526 |
| yn_gRN | + 4 | AGCC | | | ggTTgaGaagagcaTcggTcTgaaggaTgc | |
| A057 | | ACGT | | | acTccgggaTagggcagTcccggcTcTTgc | |
| | | CTAC | | | TaagTccTTcagcaagTcgaaagacacgaTg | |
| | | AGCA | | | TgagccTaTTTT | |
| | | GGGC | | | | |
| IID25_S | TTR6 | GTCT | 520 | V118 | gTCTcaaTcaaccgaTagaaaTaTcggTcc | 526 |
| yn_gRN | + 4 | ACAG | | | ggTTgaGaagagcaTcggTcTgaaggaTgc | |
| A058 | | CAGG | | | acTccgggaTagggcagTcccggcTcTTgc | |
| | | GCTG | | | TaagTccTTcagcaagTcgaaagacacgaTg | |
| | | CCTC | | | TgagccTaTTTT | |
| | | GGAC | | | | |
| IID25_S | TTR7 | CTCT | 522 | V118 | gTCTcaaTcaaccgaTagaaaTaTcggTcc | 526 |
| yn_gRN | + 4 | GAGG | | | ggTTgaGaagagcaTcggTcTgaaggaTgc | |
| A059 | | GATC | | | acTccgggaTagggcagTcccggcTcTTgc | |
| | | CTGG | | | TaagTccTTcagcaagTcgaaagacacgaTg | |
| | | GAGC | | | TgagccTaTTTT | |
| | | CCTT | | | | |
| IID25_S | Tet | CTCC | 516 | v70 | gtttcaatcaaccgatagaaatatcggtccggttgaa | 524 |
| yn_gRN | reporter | CTAT | | | aagagcatcggtctgaaggatgcactccgggatag | |
| A060 | | CAGT | | | ggcagtcccggctcttgctaagtccttcagcaagtc | |
| | | GATA | | | gaaagacacgatgtgagcctatttt | |
| | | GAGA | | | | |
| IID25_S | Tet | CTCC | 516 | V118 | gTCTcaaTcaaccgaTagaaaTaTcggTcc | 526 |
| yn_gRN | reporter | CTAT | | | ggTTgaGaagagcaTcggTcTgaaggaTgc | |
| A061 | | CAGT | | | acTccgggaTagggcagTcccggcTcTTgc | |
| | | GATA | | | TaagTccTTcagcaagTcgaaagacacgaTg | |
| | | GAGA | | | TgagccTaTTTT | |
| IID25_S | TTR1 | AGCC | 517 | V151 | GTCTCAATCAACCGATAGAAAT | 536 |
| yn_gRN | | ACGT | | | ATCGGTCCGGTTGAGAAGAGCA | |
| A062 | | CTAC | | | TCGGCTGAAGGATGCACTCCGG | |
| | | AGCA | | | GATAGGGCAGTCCCGGCTCTTG | |
| | | GGGC | | | CTAAGTCCTTCAGCAAGCGAAA | |
| | | | | | GCACGATGTGAGCCTATTTT | |
| IID25_S | TTR6 | ACAG | 519 | V151 | GTCTCAATCAACCGATAGAAAT | 536 |
| yn_gRN | | CAGG | | | ATCGGTCCGGTTGAGAAGAGCA | |
| A063 | | GCTG | | | TCGGCTGAAGGATGCACTCCGG | |
| | | CCTC | | | GATAGGGCAGTCCCGGCTCTTG | |
| | | GGAC | | | CTAAGTCCTTCAGCAAGCGAAA | |
| | | | | | GCACGATGTGAGCCTATTTT | |
| IID25_S | TTR7 | GAGG | 521 | V151 | GTCTCAATCAACCGATAGAAAT | 536 |
| yn_gRN | | GATC | | | ATCGGTCCGGTTGAGAAGAGCA | |
| A064 | | CTGG | | | TCGGCTGAAGGATGCACTCCGG | |
| | | GAGC | | | GATAGGGCAGTCCCGGCTCTTG | |
| | | CCTT | | | CTAAGTCCTTCAGCAAGCGAAA | |
| | | | | | GCACGATGTGAGCCTATTTT | |
| IID25_S | TTR1 | AGCC | 517 | V151 | GTCTCAATCAACCGATAGAAAT | 536 |
| yn_gRN | | ACGT | | | ATCGGTCCGGTTGAGAAGAGCA | |
| A065 | | CTAC | | | TCGGCTGAAGGATGCACTCCGG | |
| | | AGCA | | | GATAGGGCAGTCCCGGCTCTTG | |
| | | GGGC | | | CTAAGTCCTTCAGCAAGCGAAA | |
| | | | | | GCACGATGTGAGCCTATTTT | |
| IID25_S | TTR6 | ACAG | 519 | V151 | GTCTCAATCAACCGATAGAAAT | 536 |
| yn_gRN | | CAGG | | | ATCGGTCCGGTTGAGAAGAGCA | |
| A066 | | GCTG | | | TCGGCTGAAGGATGCACTCCGG | |
| | | CCTC | | | GATAGGGCAGTCCCGGCTCTTG | |
| | | GGAC | | | CTAAGTCCTTCAGCAAGCGAAA | |
| | | | | | GCACGATGTGAGCCTATTTT | |
| IID25_S | TTR7 | GAGG | 521 | V151 | GTCTCAATCAACCGATAGAAAT | 536 |
| yn_gRN | | GATC | | | ATCGGTCCGGTTGAGAAGAGCA | |
| A067 | | CTGG | | | TCGGCTGAAGGATGCACTCCGG | |
| | | GAGC | | | GATAGGGCAGTCCCGGCTCTTG | |
| | | CCTT | | | CTAAGTCCTTCAGCAAGCGAAA | |
| | | | | | GCACGATGTGAGCCTATTTT | |
| IID25_S | TTR1 | AGCC | 517 | V118 | gTCTcaaTcaaccgaTagaaaTaTcggTcc | 526 |
| yn_gRN | | ACGT | | | ggTTgaGaagagcaTcggTcTgaaggaTgc | |
| A068 | | CTAC | | | acTccgggaTagggcagTcccggcTcTTgc | |
| | | AGCA | | | TaagTccTTcagcaagTcgaaagacacgaTg | |
| | | GGGC | | | TgagccTaTTTT | |
| IID25_S | TTR6 | ACAG | 519 | V118 | gTCTcaaTcaaccgaTagaaaTaTcggTcc | 526 |
| yn_gRN | | CAGG | | | ggTTgaGaagagcaTcggTcTgaaggaTgc | |
| A069 | | GCTG | | | acTccgggaTagggcagTcccggcTcTTgc | |
| | | CCTC | | | TaagTccTTcagcaagTcgaaagacacgaTg | |
| | | GGAC | | | TgagccTaTTTT | |
| IID25_S | TTR7 | GAGG | 521 | V118 | gTCTcaaTcaaccgaTagaaaTaTcggTcc | 526 |
| yn_gRN | | GATC | | | ggTTgaGaagagcaTcggTcTgaaggaTgc | |
| A070 | | CTGG | | | acTccgggaTagggcagTcccggcTcTTgc | |
| | | GAGC | | | TaagTccTTcagcaagTcgaaagacacgaTg | |
| | | CCTT | | | TgagccTaTTTT | |
| IID25_S | TTR1 | TTAC | 518 | V118 | gTCTcaaTcaaccgaTagaaaTaTcggTcc | 526 |
| yn_gRN | + 4 | AGCC | | | ggTTgaGaagagcaTcggTcTgaaggaTgc | |
| A071 | | ACGT | | | acTccgggaTagggcagTcccggcTcTTgc | |
| | | CTAC | | | TaagTccTTcagcaagTcgaaagacacgaTg | |
| | | AGCA | | | TgagccTaTTTT | |
| | | GGGC | | | | |
| IID25_S | TTR6 | GTCT | 520 | V118 | gTCTcaaTcaaccgaTagaaaTaTcggTcc | 526 |
| yn_gRN | + 4 | ACAG | | | ggTTgaGaagagcaTcggTcTgaaggaTgc | |
| A072 | | CAGG | | | acTccgggaTagggcagTcccggcTcTTgc | |
| | | GCTG | | | TaagTccTTcagcaagTcgaaagacacgaTg | |
| | | CCTC | | | TgagccTaTTTT | |
| | | GGAC | | | | |
| IID25_S | TTR7 | CTCT | 522 | V118 | gTCTcaaTcaaccgaTagaaaTaTcggTcc | 526 |
| yn_gRN | + 4 | GAGG | | | ggTTgaGaagagcaTcggTcTgaaggaTgc | |
| A073 | | GATC | | | acTccgggaTagggcagTcccggcTcTTgc | |
| | | CTGG | | | TaagTccTTcagcaagTcgaaagacacgaTg | |
| | | GAGC | | | TgagccTaTTTT | |
| | | CCTT | | | | |
| | | | | V119 | gGuucaaucaaccgauagaaauaucgguccgg | 772 |
| | | | | | uugaaCagagcaucggucugaaggaugcacuc | |
| | | | | | cgggauagggcagucccggcucuugcuaaguc | |
| | | | | | cuucagcaagucgaaagacacgaugugagccu | |

TABLE 7B
Exemplary DNA Sequences of Guides:
SI# indicates the corresponding SEQ ID NO.

| | Target | Scaffold | | | |
|---|---|---|---|---|---|
| RNA ID | ID | ID | sgRNA Seq (DNA) | SI# | LEN |
| IID25_S | EMX1-2 | v70 | CTCCAATGACTAGGGTGGGCGTTTCAATCA | 538 | 153 |
| yn_gRN | | | ACCGATAGAAATATCGGTCCGGTTGAAAAG | | |
| A001 | | | AGCATCGGTCTGAAGGATGCACTCCGGGAT | | |
| | | | AGGGCAGTCCCGGCTCTTGCTAAGTCCTTCA | | |
| | | | GCAAGTCGAAAGACACGATGTGAGCCTATT | | |
| | | | TT | | |
| IID25_S | EMX1-4 | v70 | ATGGGCTGGCTGAGGCCTGGGTTTCAATCA | 539 | 153 |
| yn_gRN | | | ACCGATAGAAATATCGGTCCGGTTGAAAAG | | |
| A002 | | | AGCATCGGTCTGAAGGATGCACTCCGGGAT | | |
| | | | AGGGCAGTCCCGGCTCTTGCTAAGTCCTTCA | | |
| | | | GCAAGTCGAAAGACACGATGTGAGCCTATT | | |
| | | | TT | | |
| IID25_S | HPRT38 | v70 | AGACAAGGCACTTGGTTGATGTTTCAATCA | 540 | 153 |
| yn_gRN | 07 | | ACCGATAGAAATATCGGTCCGGTTGAAAAG | | |
| A003 | | | AGCATCGGTCTGAAGGATGCACTCCGGGAT | | |
| | | | AGGGCAGTCCCGGCTCTTGCTAAGTCCTTCA | | |
| | | | GCAAGTCGAAAGACACGATGTGAGCCTATT | | |
| | | | TT | | |
| IID25_S | VEGFA1 | v70 | CCCACACGCACACACTCACTGTTTCAATCAA | 541 | 153 |
| yn_gRN | | | CCGATAGAAATATCGGTCCGGTTGAAAAGA | | |
| A004 | | | GCATCGGTCTGAAGGATGCACTCCGGGATA | | |
| | | | GGGCAGTCCCGGCTCTTGCTAAGTCCTTCAG | | |
| | | | CAAGTCGAAAGACACGATGTGAGCCTATTT | | |
| | | | T | | |
| IID25_S | IID25_P | v70 | GACTTGGGTGAGCGATGGACGTTTCAATCA | 542 | 153 |
| yn_gRN | T_5_TT | | ACCGATAGAAATATCGGTCCGGTTGAAAAG | | |
| A005 | LL10 | | AGCATCGGTCTGAAGGATGCACTCCGGGAT | | |
| | | | AGGGCAGTCCCGGCTCTTGCTAAGTCCTTCA | | |
| | | | GCAAGTCGAAAGACACGATGTGAGCCTATT | | |
| | | | TT | | |
| IID25_S | IID25_P | v70 | GGACCTCCAGGTGCAGAAGTGTTTCAATCA | 543 | 153 |
| yn_gRN | T_10_C1 | | ACCGATAGAAATATCGGTCCGGTTGAAAAG | | |
| A006 | QTNF12 | | AGCATCGGTCTGAAGGATGCACTCCGGGAT | | |
| | | | AGGGCAGTCCCGGCTCTTGCTAAGTCCTTCA | | |
| | | | GCAAGTCGAAAGACACGATGTGAGCCTATT | | |
| | | | TT | | |
| IID25_S | IID25_P | v70 | CTGTGAAGAGCCAGTGGCCAGTTTCAATCA | 544 | 153 |
| yn_gRN | T_15_CP | | ACCGATAGAAATATCGGTCCGGTTGAAAAG | | |
| A007 | TP | | AGCATCGGTCTGAAGGATGCACTCCGGGAT | | |
| | | | AGGGCAGTCCCGGCTCTTGCTAAGTCCTTCA | | |
| | | | GCAAGTCGAAAGACACGATGTGAGCCTATT | | |
| | | | TT | | |
| IID25_S | IID25_P | v70 | GGACAAAGGTCCTCAAAGACGTTTCAATCA | 545 | 153 |
| yn_gRN | T_22_V | | ACCGATAGAAATATCGGTCCGGTTGAAAAG | | |
| A008 | WA1 | | AGCATCGGTCTGAAGGATGCACTCCGGGAT | | |
| | | | AGGGCAGTCCCGGCTCTTGCTAAGTCCTTCA | | |
| | | | GCAAGTCGAAAGACACGATGTGAGCCTATT | | |
| | | | TT | | |
| IID25_S | IID25_P | V117 | GACTTGGGTGAGCGATGGACGCTTCAATCA | 546 | 153 |
| yn_gRN | T_5_TT | | ACCGATAGAAATATCGGTCCGGTTGAAGAG | | |
| A009 | LL10 | | AGCATCGGTCTGAAGGATGCACTCCGGGAT | | |
| | | | AGGGCAGTCCCGGCTCTTGCTAAGTCCTTCA | | |
| | | | GCAAGTCGAAAGACACGATGTGAGCCTATT | | |
| | | | TT | | |
| IID25_S | IID25_P | V118 | GACTTGGGTGAGCGATGGACGTCTCAATCA | 547 | 153 |
| yn_gRN | T_5_TT | | ACCGATAGAAATATCGGTCCGGTTGAGAAG | | |
| A010 | LL10 | | AGCATCGGTCTGAAGGATGCACTCCGGGAT | | |
| | | | AGGGCAGTCCCGGCTCTTGCTAAGTCCTTCA | | |
| | | | GCAAGTCGAAAGACACGATGTGAGCCTATT | | |
| | | | TT | | |
| IID25_S | IID25_P | V123 | GACTTGGGTGAGCGATGGACGTTTCGATCA | 548 | 153 |
| yn_gRN | T_5_TT | | ACCGATAGAAATATCGGTCCGGTCGAAAAG | | |
| A011 | LL10 | | AGCATCGGTCTGAAGGATGCACTCCGGGAT | | |
| | | | AGGGCAGTCCCGGCTCTTGCTAAGTCCTTCA | | |
| | | | GCAAGTCGAAAGACACGATGTGAGCCTATT | | |
| | | | TT | | |
| IID25_S | IID25_P | V126 | GACTTGGGTGAGCGATGGACGTTTCAATCA | 549 | 153 |
| yn_gRN | T_5_TT | | GCCGATAGAAATATCGGCCCGGTTGAAAAG | | |
| A012 | LL10 | | AGCATCGGTCTGAAGGATGCACTCCGGGAT | | |
| | | | AGGGCAGTCCCGGCTCTTGCTAAGTCCTTCA | | |
| | | | GCAAGTCGAAAGACACGATGTGAGCCTATT | | |
| | | | TT | | |
| IID25_S | IID25_P | V127 | GACTTGGGTGAGCGATGGACGTTTCAATCA | 550 | 153 |
| yn_gRN | T_5_TT | | CCCGATAGAAATATCGGGCCGGTTGAAAAG | | |
| A013 | LL10 | | AGCATCGGTCTGAAGGATGCACTCCGGGAT | | |
| | | | AGGGCAGTCCCGGCTCTTGCTAAGTCCTTCA | | |
| | | | GCAAGTCGAAAGACACGATGTGAGCCTATT | | |
| | | | TT | | |
| IID25_S | IID25_P | V128 | GACTTGGGTGAGCGATGGACGTTTCAATCA | 551 | 151 |
| yn_gRN | T_5_TT | | ACCGATGAAAATCGGTCCGGTTGAAAAGAG | | |
| A014 | LL10 | | CATCGGTCTGAAGGATGCACTCCGGGATAG | | |
| | | | GGCAGTCCCGGCTCTTGCTAAGTCCTTCAGC | | |
| | | | AAGTCGAAAGACACGATGTGAGCCTATTTT | | |
| IID25_S | IID25_P | V129 | GACTTGGGTGAGCGATGGACGTTTCAATCA | 552 | 149 |
| yn_gRN | T_5_TT | | ACCGAGAAATCGGTCCGGTTGAAAAGAGCA | | |
| A015 | LL10 | | TCGGTCTGAAGGATGCACTCCGGGATAGGG | | |
| | | | CAGTCCCGGCTCTTGCTAAGTCCTTCAGCAA | | |
| | | | GTCGAAAGACACGATGTGAGCCTATTTT | | |
| IID25_S | IID25_P | V130 | GACTTGGGTGAGCGATGGACGTTTCAATCA | 553 | 152 |
| yn_gRN | T_5_TT | | ACCGATAGAAATATCGGTCCGGTTGAAAAG | | |
| A016 | LL10 | | AGCATCGGCTGAAGGATGCACTCCGGGATA | | |
| | | | GGGCAGTCCCGGCTCTTGCTAAGTCCTTCAG | | |
| | | | CAAGTCGAAAGACACGATGTGAGCCTATTT | | |
| | | | T | | |
| IID25_S | IID25_P | V134 | GACTTGGGTGAGCGATGGACGTTTCAATCA | 554 | 151 |
| yn_gRN | T_5_TT | | ACCGATAGAAATATCGGTCCGGTTGAAAAG | | |
| A017 | LL10 | | AGCATCGGTCTGAAGGATGCACTCCGGGAT | | |
| | | | AGGGCAGTCCCGGCTCTTGCTAAGTCCTTCA | | |
| | | | GCAAGCGAAAGCACGATGTGAGCCTATTTT | | |
| IID25_S | IID25_P | V117 | GGACCTCCAGGTGCAGAAGTGCTTCAATCA | 555 | 153 |
| yn_gRN | T_10_C1 | | ACCGATAGAAATATCGGTCCGGTTGAAGAG | | |
| A018 | QTNF12 | | AGCATCGGTCTGAAGGATGCACTCCGGGAT | | |
| | | | AGGGCAGTCCCGGCTCTTGCTAAGTCCTTCA | | |
| | | | GCAAGTCGAAAGACACGATGTGAGCCTATT | | |
| | | | TT | | |
| IID25_S | IID25_P | V118 | GGACCTCCAGGTGCAGAAGTGTCTCAATCA | 556 | 153 |
| yn_gRN | T_10_C1 | | ACCGATAGAAATATCGGTCCGGTTGAGAAG | | |
| A019 | QTNF12 | | AGCATCGGTCTGAAGGATGCACTCCGGGAT | | |
| | | | AGGGCAGTCCCGGCTCTTGCTAAGTCCTTCA | | |
| | | | GCAAGTCGAAAGACACGATGTGAGCCTATT | | |
| | | | TT | | |
| IID25_S | IID25_P | V123 | GGACCTCCAGGTGCAGAAGTGTTTCGATCA | 557 | 153 |
| yn_gRN | T_10_C1 | | ACCGATAGAAATATCGGTCCGGTCGAAAAG | | |
| A020 | QTNF12 | | AGCATCGGTCTGAAGGATGCACTCCGGGAT | | |
| | | | AGGGCAGTCCCGGCTCTTGCTAAGTCCTTCA | | |
| | | | GCAAGTCGAAAGACACGATGTGAGCCTATT | | |
| | | | TT | | |
| IID25_S | IID25_P | V126 | GGACCTCCAGGTGCAGAAGTGTTTCAATCA | 558 | 153 |
| yn_gRN | T_10_C1 | | GCCGATAGAAATATCGGCCCGGTTGAAAAG | | |
| A021 | QTNF12 | | AGCATCGGTCTGAAGGATGCACTCCGGGAT | | |
| | | | AGGGCAGTCCCGGCTCTTGCTAAGTCCTTCA | | |
| | | | GCAAGTCGAAAGACACGATGTGAGCCTATT | | |
| | | | TT | | |
| IID25_S | IID25_P | V127 | GGACCTCCAGGTGCAGAAGTGTTTCAATCA | 559 | 153 |
| yn_gRN | T_10_C1 | | CCCGATAGAAATATCGGGCCGGTTGAAAAG | | |
| A022 | QTNF12 | | AGCATCGGTCTGAAGGATGCACTCCGGGAT | | |
| | | | AGGGCAGTCCCGGCTCTTGCTAAGTCCTTCA | | |
| | | | GCAAGTCGAAAGACACGATGTGAGCCTATT | | |
| | | | TT | | |
| IID25_S | IID25_P | V128 | GGACCTCCAGGTGCAGAAGTGTTTCAATCA | 560 | 151 |
| yn_gRN | T_10_C1 | | ACCGATGAAAATCGGTCCGGTTGAAAAGAG | | |
| A023 | QTNF12 | | CATCGGTCTGAAGGATGCACTCCGGGATAG | | |
| | | | GGCAGTCCCGGCTCTTGCTAAGTCCTTCAGC | | |
| | | | AAGTCGAAAGACACGATGTGAGCCTATTTT | | |
| IID25_S | IID25_P | V129 | GGACCTCCAGGTGCAGAAGTGTTTCAATCA | 561 | 149 |
| yn_gRN | T_10_C1 | | ACCGAGAAATCGGTCCGGTTGAAAAGAGCA | | |
| A024 | QTNF12 | | TCGGTCTGAAGGATGCACTCCGGGATAGGG | | |
| | | | CAGTCCCGGCTCTTGCTAAGTCCTTCAGCAA | | |
| | | | GTCGAAAGACACGATGTGAGCCTATTTT | | |
| IID25_S | IID25_P | V130 | GGACCTCCAGGTGCAGAAGTGTTTCAATCA | 562 | 152 |
| yn_gRN | T_10_C1 | | ACCGATAGAAATATCGGTCCGGTTGAAAAG | | |
| A025 | QTNF12 | | AGCATCGGCTGAAGGATGCACTCCGGGATA | | |
| | | | GGGCAGTCCCGGCTCTTGCTAAGTCCTTCAG | | |
| | | | CAAGTCGAAAGACACGATGTGAGCCTATTT | | |
| | | | T | | |
| IID25_S | IID25_P | V134 | GGACCTCCAGGTGCAGAAGTGTTTCAATCA | 563 | 151 |
| yn_gRN | T_10_C1 | | ACCGATAGAAATATCGGTCCGGTTGAAAAG | | |
| A026 | QTNF12 | | AGCATCGGTCTGAAGGATGCACTCCGGGAT | | |
| | | | AGGGCAGTCCCGGCTCTTGCTAAGTCCTTCA | | |
| | | | GCAAGCGAAAGCACGATGTGAGCCTATTTT | | |
| IID25_S | IID25_P | V117 | CTGTGAAGAGCCAGTGGCCAGCTTCAATCA | 564 | 153 |
| yn_gRN | T_15_CP | | ACCGATAGAAATATCGGTCCGGTTGAAGAG | | |
| A027 | TP | | AGCATCGGTCTGAAGGATGCACTCCGGGAT | | |
| | | | AGGGCAGTCCCGGCTCTTGCTAAGTCCTTCA | | |
| | | | GCAAGTCGAAAGACACGATGTGAGCCTATT | | |
| | | | TT | | |
| IID25_S | IID25_P | V118 | CTGTGAAGAGCCAGTGGCCAGTCTCAATCA | 565 | 153 |
| yn_gRN | T_15_CP | | ACCGATAGAAATATCGGTCCGGTTGAGAAG | | |
| A028 | TP | | AGCATCGGTCTGAAGGATGCACTCCGGGAT | | |
| | | | AGGGCAGTCCCGGCTCTTGCTAAGTCCTTCA | | |
| | | | GCAAGTCGAAAGACACGATGTGAGCCTATT | | |
| | | | TT | | |
| IID25_S | IID25_P | V123 | CTGTGAAGAGCCAGTGGCCAGTTTCGATCA | 566 | 153 |
| yn_gRN | T_15_CP | | ACCGATAGAAATATCGGTCCGGTCGAAAAG | | |
| A029 | TP | | AGCATCGGTCTGAAGGATGCACTCCGGGAT | | |
| | | | AGGGCAGTCCCGGCTCTTGCTAAGTCCTTCA | | |
| | | | GCAAGTCGAAAGACACGATGTGAGCCTATT | | |
| | | | TT | | |
| IID25_S | IID25_P | V126 | CTGTGAAGAGCCAGTGGCCAGTTTCAATCA | 567 | 153 |
| yn_gRN | T_15_CP | | GCCGATAGAAATATCGGCCCGGTTGAAAAG | | |
| A030 | TP | | AGCATCGGTCTGAAGGATGCACTCCGGGAT | | |
| | | | AGGGCAGTCCCGGCTCTTGCTAAGTCCTTCA | | |
| | | | GCAAGTCGAAAGACACGATGTGAGCCTATT | | |
| | | | TT | | |
| IID25_S | IID25_P | V127 | CTGTGAAGAGCCAGTGGCCAGTTTCAATCA | 568 | 153 |
| yn_gRN | T_15_CP | | CCCGATAGAAATATCGGGCCGGTTGAAAAG | | |
| A031 | TP | | AGCATCGGTCTGAAGGATGCACTCCGGGAT | | |
| | | | AGGGCAGTCCCGGCTCTTGCTAAGTCCTTCA | | |
| | | | GCAAGTCGAAAGACACGATGTGAGCCTATT | | |
| | | | TT | | |
| IID25_S | IID25_P | V128 | CTGTGAAGAGCCAGTGGCCAGTTTCAATCA | 569 | 151 |
| yn_gRN | T_15_CP | | ACCGATGAAAATCGGTCCGGTTGAAAAGAG | | |
| A032 | TP | | CATCGGTCTGAAGGATGCACTCCGGGATAG | | |
| | | | GGCAGTCCCGGCTCTTGCTAAGTCCTTCAGC | | |
| | | | AAGTCGAAAGACACGATGTGAGCCTATTTT | | |
| IID25_S | IID25_P | V129 | CTGTGAAGAGCCAGTGGCCAGTTTCAATCA | 570 | 149 |
| yn_gRN | T_15_CP | | ACCGAGAAATCGGTCCGGTTGAAAAGAGCA | | |
| A033 | TP | | TCGGTCTGAAGGATGCACTCCGGGATAGGG | | |
| | | | CAGTCCCGGCTCTTGCTAAGTCCTTCAGCAA | | |
| | | | GTCGAAAGACACGATGTGAGCCTATTTT | | |
| IID25_S | IID25_P | V130 | CTGTGAAGAGCCAGTGGCCAGTTTCAATCA | 571 | 152 |
| yn_gRN | T_15_CP | | ACCGATAGAAATATCGGTCCGGTTGAAAAG | | |
| A034 | TP | | AGCATCGGCTGAAGGATGCACTCCGGGATA | | |
| | | | GGGCAGTCCCGGCTCTTGCTAAGTCCTTCAG | | |
| | | | CAAGTCGAAAGACACGATGTGAGCCTATTT | | |
| | | | T | | |
| IID25_S | IID25_P | V134 | CTGTGAAGAGCCAGTGGCCAGTTTCAATCA | 572 | 151 |
| yn_gRN | T_15_CP | | ACCGATAGAAATATCGGTCCGGTTGAAAAG | | |
| A035 | TP | | AGCATCGGTCTGAAGGATGCACTCCGGGAT | | |
| | | | AGGGCAGTCCCGGCTCTTGCTAAGTCCTTCA | | |
| | | | GCAAGCGAAAGCACGATGTGAGCCTATTTT | | |
| IID25_S | IID25_P | V149 | GACTTGGGTGAGCGATGGACGCCTCAATCA | 573 | 153 |
| yn_gRN | T_5_TT | | ACCGATAGAAATATCGGTCCGGTTGAGGAG | | |
| A036 | LL10 | | AGCATCGGTCTGAAGGATGCACTCCGGGAT | | |
| | | | AGGGCAGTCCCGGCTCTTGCTAAGTCCTTCA | | |
| | | | GCAAGTCGAAAGACACGATGTGAGCCTATT | | |
| | | | TT | | |
| IID25_S | IID25_P | V150 | GACTTGGGTGAGCGATGGACGCCTCGATCA | 574 | 153 |
| yn_gRN | T_5_TT | | ACCGATAGAAATATCGGTCCGGTCGAGGAG | | |
| A037 | LL10 | | AGCATCGGTCTGAAGGATGCACTCCGGGAT | | |
| | | | AGGGCAGTCCCGGCTCTTGCTAAGTCCTTCA | | |
| | | | GCAAGTCGAAAGACACGATGTGAGCCTATT | | |
| | | | TT | | |
| IID25_S | IID25_P | V151 | GACTTGGGTGAGCGATGGACGTCTCAATCA | 575 | 150 |
| yn_gRN | T_5_TT | | ACCGATAGAAATATCGGTCCGGTTGAGAAG | | |
| A038 | LL10 | | AGCATCGGCTGAAGGATGCACTCCGGGATA | | |
| | | | GGGCAGTCCCGGCTCTTGCTAAGTCCTTCAG | | |
| | | | CAAGCGAAAGCACGATGTGAGCCTATTTT | | |
| IID25_S | IID25_P | V152 | GACTTGGGTGAGCGATGGACGTCTCAATCA | 576 | 146 |
| yn_gRN | T_5_TT | | ACCGAGAAATCGGTCCGGTTGAAAAGAGCA | | |
| A039 | LL10 | | TCGGCTGAAGGATGCACTCCGGGATAGGGC | | |
| | | | AGTCCCGGCTCTTGCTAAGTCCTTCAGCAAG | | |
| | | | CGAAAGCACGATGTGAGCCTATTTT | | |
| IID25_S | IID25_P | V149 | GGACCTCCAGGTGCAGAAGTGCCTCAATCA | 577 | 153 |
| yn_gRN | T_10_C1 | | ACCGATAGAAATATCGGTCCGGTTGAGGAG | | |
| A040 | QTNF12 | | AGCATCGGTCTGAAGGATGCACTCCGGGAT | | |
| | | | AGGGCAGTCCCGGCTCTTGCTAAGTCCTTCA | | |
| | | | GCAAGTCGAAAGACACGATGTGAGCCTATT | | |
| | | | TT | | |
| IID25_S | IID25_P | V150 | GGACCTCCAGGTGCAGAAGTGCCTCGATCA | 578 | 153 |
| yn_gRN | T_10_C1 | | ACCGATAGAAATATCGGTCCGGTCGAGGAG | | |
| A041 | QTNF12 | | AGCATCGGTCTGAAGGATGCACTCCGGGAT | | |
| | | | AGGGCAGTCCCGGCTCTTGCTAAGTCCTTCA | | |
| | | | GCAAGTCGAAAGACACGATGTGAGCCTATT | | |
| | | | TT | | |
| IID25_S | IID25_P | V151 | GGACCTCCAGGTGCAGAAGTGTCTCAATCA | 579 | 150 |
| yn_gRN | T_10_C1 | | ACCGATAGAAATATCGGTCCGGTTGAGAAG | | |
| A042 | QTNF12 | | AGCATCGGCTGAAGGATGCACTCCGGGATA | | |
| | | | GGGCAGTCCCGGCTCTTGCTAAGTCCTTCAG | | |
| | | | CAAGCGAAAGCACGATGTGAGCCTATTTT | | |
| IID25_S | IID25_P | V152 | GGACCTCCAGGTGCAGAAGTGTCTCAATCA | 580 | 146 |
| yn_gRN | T_10_C1 | | ACCGAGAAATCGGTCCGGTTGAAAAGAGCA | | |
| A043 | QTNF12 | | TCGGCTGAAGGATGCACTCCGGGATAGGGC | | |
| | | | AGTCCCGGCTCTTGCTAAGTCCTTCAGCAAG | | |
| | | | CGAAAGCACGATGTGAGCCTATTTT | | |
| IID25_S | IID25_P | V149 | CTGTGAAGAGCCAGTGGCCAGCCTCAATCA | 581 | 153 |
| yn_gRN | T_15_CP | | ACCGATAGAAATATCGGTCCGGTTGAGGAG | | |
| A044 | TP | | AGCATCGGTCTGAAGGATGCACTCCGGGAT | | |
| | | | AGGGCAGTCCCGGCTCTTGCTAAGTCCTTCA | | |
| | | | GCAAGTCGAAAGACACGATGTGAGCCTATT | | |
| | | | TT | | |
| IID25_S | IID25_P | V150 | CTGTGAAGAGCCAGTGGCCAGCCTCGATCA | 582 | 153 |
| yn_gRN | T_15_CP | | ACCGATAGAAATATCGGTCCGGTCGAGGAG | | |
| A045 | TP | | AGCATCGGTCTGAAGGATGCACTCCGGGAT | | |
| | | | AGGGCAGTCCCGGCTCTTGCTAAGTCCTTCA | | |
| | | | GCAAGTCGAAAGACACGATGTGAGCCTATT | | |
| | | | TT | | |
| IID25_S | IID25_P | V151 | CTGTGAAGAGCCAGTGGCCAGTCTCAATCA | 583 | 150 |
| yn_gRN | T_15_CP | | ACCGATAGAAATATCGGTCCGGTTGAGAAG | | |
| A046 | TP | | AGCATCGGCTGAAGGATGCACTCCGGGATA | | |
| | | | GGGCAGTCCCGGCTCTTGCTAAGTCCTTCAG | | |
| | | | CAAGCGAAAGCACGATGTGAGCCTATTTT | | |
| IID25_S | IID25_P | V152 | CTGTGAAGAGCCAGTGGCCAGTCTCAATCA | 584 | 146 |
| yn_gRN | T_15_CP | | ACCGAGAAATCGGTCCGGTTGAAAAGAGCA | | |
| A047 | TP | | TCGGCTGAAGGATGCACTCCGGGATAGGGC | | |
| | | | AGTCCCGGCTCTTGCTAAGTCCTTCAGCAAG | | |
| | | | CGAAAGCACGATGTGAGCCTATTTT | | |
| IID25_S | TTR1 | v70 | AGCCACGTCTACAGCAGGGCGTTTCAATCA | 585 | 153 |
| yn_gRN | | | ACCGATAGAAATATCGGTCCGGTTGAAAAG | | |
| A048 | | | AGCATCGGTCTGAAGGATGCACTCCGGGAT | | |
| | | | AGGGCAGTCCCGGCTCTTGCTAAGTCCTTCA | | |
| | | | GCAAGTCGAAAGACACGATGTGAGCCTATT | | |
| | | | TT | | |
| IID25_S | TTR6 | v70 | ACAGCAGGGCTGCCTCGGACGTTTCAATCA | 586 | 153 |
| yn_gRN | | | ACCGATAGAAATATCGGTCCGGTTGAAAAG | | |
| A049 | | | AGCATCGGTCTGAAGGATGCACTCCGGGAT | | |
| | | | AGGGCAGTCCCGGCTCTTGCTAAGTCCTTCA | | |
| | | | GCAAGTCGAAAGACACGATGTGAGCCTATT | | |
| | | | TT | | |
| IID25_S | TTR7 | v70 | GAGGGATCCTGGGAGCCCTTGTTTCAATCA | 587 | 153 |
| yn_gRN | | | ACCGATAGAAATATCGGTCCGGTTGAAAAG | | |
| A050 | | | AGCATCGGTCTGAAGGATGCACTCCGGGAT | | |
| | | | AGGGCAGTCCCGGCTCTTGCTAAGTCCTTCA | | |
| | | | GCAAGTCGAAAGACACGATGTGAGCCTATT | | |
| | | | TT | | |
| IID25_S | TTR1 | V118 | AGCCACGTCTACAGCAGGGCGTCTCAATCA | 588 | 153 |
| yn_gRN | | | ACCGATAGAAATATCGGTCCGGTTGAGAAG | | |
| A051 | | | AGCATCGGTCTGAAGGATGCACTCCGGGAT | | |
| | | | AGGGCAGTCCCGGCTCTTGCTAAGTCCTTCA | | |
| | | | GCAAGTCGAAAGACACGATGTGAGCCTATT | | |
| | | | TT | | |
| IID25_S | TTR6 | V118 | ACAGCAGGGCTGCCTCGGACGTCTCAATCA | 589 | 153 |
| yn_gRN | | | ACCGATAGAAATATCGGTCCGGTTGAGAAG | | |
| A052 | | | AGCATCGGTCTGAAGGATGCACTCCGGGAT | | |
| | | | AGGGCAGTCCCGGCTCTTGCTAAGTCCTTCA | | |
| | | | GCAAGTCGAAAGACACGATGTGAGCCTATT | | |
| | | | TT | | |
| IID25_S | TTR7 | V118 | GAGGGATCCTGGGAGCCCTTGTCTCAATCA | 590 | 153 |
| yn_gRN | | | ACCGATAGAAATATCGGTCCGGTTGAGAAG | | |
| A053 | | | AGCATCGGTCTGAAGGATGCACTCCGGGAT | | |
| | | | AGGGCAGTCCCGGCTCTTGCTAAGTCCTTCA | | |
| | | | GCAAGTCGAAAGACACGATGTGAGCCTATT | | |
| | | | TT | | |
| IID25_S | TTR1 + 4 | v70 | TTACAGCCACGTCTACAGCAGGGCGTTTCA | 591 | 157 |
| yn_gRN | | | ATCAACCGATAGAAATATCGGTCCGGTTGA | | |
| A054 | | | AAAGAGCATCGGTCTGAAGGATGCACTCCG | | |
| | | | GGATAGGGCAGTCCCGGCTCTTGCTAAGTC | | |
| | | | CTTCAGCAAGTCGAAAGACACGATGTGAGC | | |
| | | | CTATTTT | | |
| IID25_S | TTR6 + 4 | v70 | GTCTACAGCAGGGCTGCCTCGGACGTTTCA | 592 | 157 |
| yn_gRN | | | ATCAACCGATAGAAATATCGGTCCGGTTGA | | |
| A055 | | | AAAGAGCATCGGTCTGAAGGATGCACTCCG | | |
| | | | GGATAGGGCAGTCCCGGCTCTTGCTAAGTC | | |
| | | | CTTCAGCAAGTCGAAAGACACGATGTGAGC | | |
| | | | CTATTTT | | |
| IID25_S | TTR7 + 4 | v70 | CTCTGAGGGATCCTGGGAGCCCTTGTTTCAA | 593 | 157 |
| yn_gRN | | | TCAACCGATAGAAATATCGGTCCGGTTGAA | | |
| A056 | | | AAGAGCATCGGTCTGAAGGATGCACTCCGG | | |
| | | | GATAGGGCAGTCCCGGCTCTTGCTAAGTCCT | | |
| | | | TCAGCAAGTCGAAAGACACGATGTGAGCCT | | |
| | | | ATTTT | | |
| IID25_S | TTR1 + 4 | V118 | TTACAGCCACGTCTACAGCAGGGCGTCTCA | 594 | 157 |
| yn_gRN | | | ATCAACCGATAGAAATATCGGTCCGGTTGA | | |
| A057 | | | GAAGAGCATCGGTCTGAAGGATGCACTCCG | | |
| | | | GGATAGGGCAGTCCCGGCTCTTGCTAAGTC | | |
| | | | CTTCAGCAAGTCGAAAGACACGATGTGAGC | | |
| | | | CTATTTT | | |
| IID25_S | TTR6 + 4 | V118 | GTCTACAGCAGGGCTGCCTCGGACGTCTCA | 595 | 157 |
| yn_gRN | | | ATCAACCGATAGAAATATCGGTCCGGTTGA | | |
| A058 | | | GAAGAGCATCGGTCTGAAGGATGCACTCCG | | |
| | | | GGATAGGGCAGTCCCGGCTCTTGCTAAGTC | | |
| | | | CTTCAGCAAGTCGAAAGACACGATGTGAGC | | |
| | | | CTATTTT | | |
| IID25_S | TTR7 + 4 | V118 | CTCTGAGGGATCCTGGGAGCCCTTGTCTCAA | 596 | 157 |
| yn_gRN | | | TCAACCGATAGAAATATCGGTCCGGTTGAG | | |
| A059 | | | AAGAGCATCGGTCTGAAGGATGCACTCCGG | | |
| | | | GATAGGGCAGTCCCGGCTCTTGCTAAGTCCT | | |
| | | | TCAGCAAGTCGAAAGACACGATGTGAGCCT | | |
| | | | ATTTT | | |
| IID25_S | Tet | v70 | CTCCCTATCAGTGATAGAGAGTTTCAATCAA | 597 | 153 |
| yn_gRN | reporter | | CCGATAGAAATATCGGTCCGGTTGAAAAGA | | |
| A060 | | | GCATCGGTCTGAAGGATGCACTCCGGGATA | | |
| | | | GGGCAGTCCCGGCTCTTGCTAAGTCCTTCAG | | |
| | | | CAAGTCGAAAGACACGATGTGAGCCTATTT | | |
| | | | T | | |
| IID25_S | Tet | V118 | CTCCCTATCAGTGATAGAGAGTCTCAATCAA | 598 | 153 |
| yn_gRN | reporter | | CCGATAGAAATATCGGTCCGGTTGAGAAGA | | |
| A061 | | | GCATCGGTCTGAAGGATGCACTCCGGGATA | | |
| | | | GGGCAGTCCCGGCTCTTGCTAAGTCCTTCAG | | |
| | | | CAAGTCGAAAGACACGATGTGAGCCTATTT | | |
| | | | T | | |
| IID25_S | TTR1 | V151 | AGCCACGTCTACAGCAGGGCGTCTCAATCA | 599 | 150 |
| yn_gRN | | | ACCGATAGAAATATCGGTCCGGTTGAGAAG | | |
| A062 | | | AGCATCGGCTGAAGGATGCACTCCGGGATA | | |
| | | | GGGCAGTCCCGGCTCTTGCTAAGTCCTTCAG | | |
| | | | CAAGCGAAAGCACGATGTGAGCCTATTTT | | |
| IID25_S | TTR6 | V151 | ACAGCAGGGCTGCCTCGGACGTCTCAATCA | 600 | 150 |
| yn_gRN | | | ACCGATAGAAATATCGGTCCGGTTGAGAAG | | |
| A063 | | | AGCATCGGCTGAAGGATGCACTCCGGGATA | | |
| | | | GGGCAGTCCCGGCTCTTGCTAAGTCCTTCAG | | |
| | | | CAAGCGAAAGCACGATGTGAGCCTATTTT | | |
| IID25_S | TTR7 | V151 | GAGGGATCCTGGGAGCCCTTGTCTCAATCA | 601 | 150 |
| yn_gRN | | | ACCGATAGAAATATCGGTCCGGTTGAGAAG | | |
| A064 | | | AGCATCGGCTGAAGGATGCACTCCGGGATA | | |
| | | | GGGCAGTCCCGGCTCTTGCTAAGTCCTTCAG | | |
| | | | CAAGCGAAAGCACGATGTGAGCCTATTTT | | |
| IID25_S | TTR1 | V151 | AGCCACGTCTACAGCAGGGCGTCTCAATCA | 602 | 150 |
| yn_gRN | | | ACCGATAGAAATATCGGTCCGGTTGAGAAG | | |
| A065 | | | AGCATCGGCTGAAGGATGCACTCCGGGATA | | |
| | | | GGGCAGTCCCGGCTCTTGCTAAGTCCTTCAG | | |
| | | | CAAGCGAAAGCACGATGTGAGCCTATTTT | | |
| IID25_S | TTR6 | V151 | ACAGCAGGGCTGCCTCGGACGTCTCAATCA | 603 | 150 |
| yn_gRN | | | ACCGATAGAAATATCGGTCCGGTTGAGAAG | | |
| A066 | | | AGCATCGGCTGAAGGATGCACTCCGGGATA | | |
| | | | GGGCAGTCCCGGCTCTTGCTAAGTCCTTCAG | | |
| | | | CAAGCGAAAGCACGATGTGAGCCTATTTT | | |
| IID25_S | TTR7 | V151 | GAGGGATCCTGGGAGCCCTTGTCTCAATCA | 604 | 150 |
| yn_gRN | | | ACCGATAGAAATATCGGTCCGGTTGAGAAG | | |
| A067 | | | AGCATCGGCTGAAGGATGCACTCCGGGATA | | |
| | | | GGGCAGTCCCGGCTCTTGCTAAGTCCTTCAG | | |
| | | | CAAGCGAAAGCACGATGTGAGCCTATTTT | | |
| IID25_S | TTR1 | V118 | AGCCACGTCTACAGCAGGGCGTCTCAATCA | 605 | 153 |
| yn_gRN | | | ACCGATAGAAATATCGGTCCGGTTGAGAAG | | |
| A068 | | | AGCATCGGTCTGAAGGATGCACTCCGGGAT | | |
| | | | AGGGCAGTCCCGGCTCTTGCTAAGTCCTTCA | | |
| | | | GCAAGTCGAAAGACACGATGTGAGCCTATT | | |
| | | | TT | | |
| IID25_S | TTR6 | V118 | ACAGCAGGGCTGCCTCGGACGTCTCAATCA | 606 | 153 |
| yn_gRN | | | ACCGATAGAAATATCGGTCCGGTTGAGAAG | | |
| A069 | | | AGCATCGGTCTGAAGGATGCACTCCGGGAT | | |
| | | | AGGGCAGTCCCGGCTCTTGCTAAGTCCTTCA | | |
| | | | GCAAGTCGAAAGACACGATGTGAGCCTATT | | |
| | | | TT | | |
| IID25_S | TTR7 | V118 | GAGGGATCCTGGGAGCCCTTGTCTCAATCA | 607 | 153 |
| yn_gRN | | | ACCGATAGAAATATCGGTCCGGTTGAGAAG | | |
| A070 | | | AGCATCGGTCTGAAGGATGCACTCCGGGAT | | |
| | | | AGGGCAGTCCCGGCTCTTGCTAAGTCCTTCA | | |
| | | | GCAAGTCGAAAGACACGATGTGAGCCTATT | | |
| | | | TT | | |
| IID25_S | TTR1 + 4 | V118 | TTACAGCCACGTCTACAGCAGGGCGTCTCA | 608 | 157 |
| yn_gRN | | | ATCAACCGATAGAAATATCGGTCCGGTTGA | | |
| A071 | | | GAAGAGCATCGGTCTGAAGGATGCACTCCG | | |
| | | | GGATAGGGCAGTCCCGGCTCTTGCTAAGTC | | |
| | | | CTTCAGCAAGTCGAAAGACACGATGTGAGC | | |
| | | | CTATTTT | | |
| IID25_S | TTR6 + 4 | V118 | GTCTACAGCAGGGCTGCCTCGGACGTCTCA | 609 | 157 |
| yn_gRN | | | ATCAACCGATAGAAATATCGGTCCGGTTGA | | |
| A072 | | | GAAGAGCATCGGTCTGAAGGATGCACTCCG | | |
| | | | GGATAGGGCAGTCCCGGCTCTTGCTAAGTC | | |
| | | | CTTCAGCAAGTCGAAAGACACGATGTGAGC | | |
| | | | CTATTTT | | |
| IID25_S | TTR7 + 4 | V118 | CTCTGAGGGATCCTGGGAGCCCTTGTCTCAA | 610 | 157 |
| yn_gRN | | | TCAACCGATAGAAATATCGGTCCGGTTGAG | | |
| A073 | | | AAGAGCATCGGTCTGAAGGATGCACTCCGG | | |
| | | | GATAGGGCAGTCCCGGCTCTTGCTAAGTCCT | | |
| | | | TCAGCAAGTCGAAAGACACGATGTGAGCCT | | |
| | | | ATTTT | | |

TABLE 7C
Exemplary RNA Sequences of Guides: SI# indicates the corresponding SEQ
ID NO; “r” indicates that the next letter is a ribonucleotide.

| | | Scaffold | | |
|---|---|---|---|---|
| RNA ID | Target ID | ID | RNA Seq | SI# |
| IID25_S | EMX1-2 | v70 | rCrUrCrCrArArUrGrArCrUrArGrGrGrUrGrGrGrCrGrUrUr | 611 |
| yn_gRN | | | UrCrArArUrCrArArCrCrGrArUrArGrArArArUrArUrCrGrG | |
| A001 | | | rUrCrCrGrGrUrUrGrArArArArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | EMX1-4 | v70 | rArUrGrGrGrCrUrGrGrCrUrGrArGrGrCrCrUrGrGrGrUrUr | 612 |
| yn_gRN | | | UrCrArArUrCrArArCrCrGrArUrArGrArArArUrArUrCrGrG | |
| A002 | | | rUrCrCrGrGrUrUrGrArArArArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | HPRT38 | v70 | rArGrArCrArArGrGrCrArCrUrUrGrGrUrUrGrArUrGrUrUr | 613 |
| yn_gRN | 07 | | UrCrArArUrCrArArCrCrGrArUrArGrArArArUrArUrCrGrG | |
| A003 | | | rUrCrCrGrGrUrUrGrArArArArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | VEGFA1 | v70 | rCrCrCrArCrArCrGrCrArCrArCrArCrUrCrArCrUrGrUrUrU | 614 |
| yn_gRN | | | rCrArArUrCrArArCrCrGrArUrArGrArArArUrArUrCrGrGr | |
| A004 | | | UrCrCrGrGrUrUrGrArArArArGrArGrCrArUrCrGrGrUrCrU | |
| | | | rGrArArGrGrArUrGrCrArCrUrCrCrGrGrGrArUrArGrGrGr | |
| | | | CrArGrUrCrCrCrGrGrCrUrCrUrUrGrCrUrArArGrUrCrCrU | |
| | | | rUrCrArGrCrArArGrUrCrGrArArArGrArCrArCrGrArUrGr | |
| | | | UrGrArGrCrCrUrArUrUrUrU | |
| IID25_S | IID25_P | v70 | rGrArCrUrUrGrGrGrUrGrArGrCrGrArUrGrGrArCrGrUrUr | 615 |
| yn_gRN | T_5_TT | | UrCrArArUrCrArArCrCrGrArUrArGrArArArUrArUrCrGrG | |
| A005 | LL10 | | rUrCrCrGrGrUrUrGrArArArArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | IID25_P | v70 | rGrGrArCrCrUrCrCrArGrGrUrGrCrArGrArArGrUrGrUrUr | 616 |
| yn_gRN | T_10_C1 | | UrCrArArUrCrArArCrCrGrArUrArGrArArArUrArUrCrGrG | |
| A006 | QTNF12 | | rUrCrCrGrGrUrUrGrArArArArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | IID25_P | v70 | rCrUrGrUrGrArArGrArGrCrCrArGrUrGrGrCrCrArGrUrUr | 617 |
| yn_gRN | T_15_CP | | UrCrArArUrCrArArCrCrGrArUrArGrArArArUrArUrCrGrG | |
| A007 | TP | | rUrCrCrGrGrUrUrGrArArArArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | IID25_P | v70 | rGrGrArCrArArArGrGrUrCrCrUrCrArArArGrArCrGrUrUr | 618 |
| yn_gRN | T_22_V | | UrCrArArUrCrArArCrCrGrArUrArGrArArArUrArUrCrGrG | |
| A008 | WA1 | | rUrCrCrGrGrUrUrGrArArArArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | IID25_P | V117 | rGrArCrUrUrGrGrGrUrGrArGrCrGrArUrGrGrArCrGrCrUr | 619 |
| yn_gRN | T_5_TT | | UrCrArArUrCrArArCrCrGrArUrArGrArArArUrArUrCrGrG | |
| A009 | LL10 | | rUrCrCrGrGrUrUrGrArArGrArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | IID25_P | V118 | rGrArCrUrUrGrGrGrUrGrArGrCrGrArUrGrGrArCrGrUrCr | 620 |
| yn_gRN | T_5_TT | | UrCrArArUrCrArArCrCrGrArUrArGrArArArUrArUrCrGrG | |
| A010 | LL10 | | rUrCrCrGrGrUrUrGrArGrArArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | IID25_P | V123 | rGrArCrUrUrGrGrGrUrGrArGrCrGrArUrGrGrArCrGrUrUr | 621 |
| yn_gRN | T_5_TT | | UrCrGrArUrCrArArCrCrGrArUrArGrArArArUrArUrCrGrG | |
| A011 | LL10 | | rUrCrCrGrGrUrCrGrArArArArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | IID25_P | V126 | rGrArCrUrUrGrGrGrUrGrArGrCrGrArUrGrGrArCrGrUrUr | 622 |
| yn_gRN | T_5_TT | | UrCrArArUrCrArGrCrCrGrArUrArGrArArArUrArUrCrGrG | |
| A012 | LL10 | | rCrCrCrGrGrUrUrGrArArArArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | IID25_P | V127 | rGrArCrUrUrGrGrGrUrGrArGrCrGrArUrGrGrArCrGrUrUr | 623 |
| yn_gRN | T_5_TT | | UrCrArArUrCrArCrCrCrGrArUrArGrArArArUrArUrCrGrG | |
| A013 | LL10 | | rGrCrCrGrGrUrUrGrArArArArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | IID25_P | V128 | rGrArCrUrUrGrGrGrUrGrArGrCrGrArUrGrGrArCrGrUrUr | 624 |
| yn_gRN | T_5_TT | | UrCrArArUrCrArArCrCrGrArUrGrArArArArUrCrGrGrUrC | |
| A014 | LL10 | | rCrGrGrUrUrGrArArArArGrArGrCrArUrCrGrGrUrCrUrGr | |
| | | | ArArGrGrArUrGrCrArCrUrCrCrGrGrGrArUrArGrGrGrCrA | |
| | | | rGrUrCrCrCrGrGrCrUrCrUrUrGrCrUrArArGrUrCrCrUrUr | |
| | | | CrArGrCrArArGrUrCrGrArArArGrArCrArCrGrArUrGrUrG | |
| | | | rArGrCrCrUrArUrUrUrU | |
| IID25_S | IID25_P | V129 | rGrArCrUrUrGrGrGrUrGrArGrCrGrArUrGrGrArCrGrUrUr | 625 |
| yn_gRN | T_5_TT | | UrCrArArUrCrArArCrCrGrArGrArArArUrCrGrGrUrCrCrG | |
| A015 | LL10 | | rGrUrUrGrArArArArGrArGrCrArUrCrGrGrUrCrUrGrArAr | |
| | | | GrGrArUrGrCrArCrUrCrCrGrGrGrArUrArGrGrGrCrArGrU | |
| | | | rCrCrCrGrGrCrUrCrUrUrGrCrUrArArGrUrCrCrUrUrCrAr | |
| | | | GrCrArArGrUrCrGrArArArGrArCrArCrGrArUrGrUrGrArG | |
| | | | rCrCrUrArUrUrUrU | |
| IID25_S | IID25_P | V130 | rGrArCrUrUrGrGrGrUrGrArGrCrGrArUrGrGrArCrGrUrUr | 626 |
| yn_gRN | T_5_TT | | UrCrArArUrCrArArCrCrGrArUrArGrArArArUrArUrCrGrG | |
| A016 | LL10 | | rUrCrCrGrGrUrUrGrArArArArGrArGrCrArUrCrGrGrCrUr | |
| | | | GrArArGrGrArUrGrCrArCrUrCrCrGrGrGrArUrArGrGrGrC | |
| | | | rArGrUrCrCrCrGrGrCrUrCrUrUrGrCrUrArArGrUrCrCrUr | |
| | | | UrCrArGrCrArArGrUrCrGrArArArGrArCrArCrGrArUrGrU | |
| | | | rGrArGrCrCrUrArUrUrUrU | |
| IID25_S | IID25_P | V134 | rGrArCrUrUrGrGrGrUrGrArGrCrGrArUrGrGrArCrGrUrUr | 627 |
| yn_gRN | T_5_TT | | UrCrArArUrCrArArCrCrGrArUrArGrArArArUrArUrCrGrG | |
| A017 | LL10 | | rUrCrCrGrGrUrUrGrArArArArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrCrGrArArArGrCrArCrGrArUrGrUrG | |
| | | | rArGrCrCrUrArUrUrUrU | |
| IID25_S | IID25_P | V117 | rGrGrArCrCrUrCrCrArGrGrUrGrCrArGrArArGrUrGrCrUr | 628 |
| yn_gRN | T_10_C1 | | UrCrArArUrCrArArCrCrGrArUrArGrArArArUrArUrCrGrG | |
| A018 | QTNF12 | | rUrCrCrGrGrUrUrGrArArGrArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | IID25_P | V118 | rGrGrArCrCrUrCrCrArGrGrUrGrCrArGrArArGrUrGrUrCr | 629 |
| yn_gRN | T_10_C1 | | UrCrArArUrCrArArCrCrGrArUrArGrArArArUrArUrCrGrG | |
| A019 | QTNF12 | | rUrCrCrGrGrUrUrGrArGrArArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | IID25_P | V123 | rGrGrArCrCrUrCrCrArGrGrUrGrCrArGrArArGrUrGrUrUr | 630 |
| yn_gRN | T_10_C1 | | UrCrGrArUrCrArArCrCrGrArUrArGrArArArUrArUrCrGrG | |
| A020 | QTNF12 | | rUrCrCrGrGrUrCrGrArArArArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | IID25_P | V126 | rGrGrArCrCrUrCrCrArGrGrUrGrCrArGrArArGrUrGrUrUr | 631 |
| yn_gRN | T_10_C1 | | UrCrArArUrCrArGrCrCrGrArUrArGrArArArUrArUrCrGrG | |
| A021 | QTNF12 | | rCrCrCrGrGrUrUrGrArArArArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | IID25_P | V127 | rGrGrArCrCrUrCrCrArGrGrUrGrCrArGrArArGrUrGrUrUr | 632 |
| yn_gRN | T_10_C1 | | UrCrArArUrCrArCrCrCrGrArUrArGrArArArUrArUrCrGrG | |
| A022 | QTNF12 | | rGrCrCrGrGrUrUrGrArArArArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | IID25_P | V128 | rGrGrArCrCrUrCrCrArGrGrUrGrCrArGrArArGrUrGrUrUr | 633 |
| yn_gRN | T_10_C1 | | UrCrArArUrCrArArCrCrGrArUrGrArArArArUrCrGrGrUrC | |
| A023 | QTNF12 | | rCrGrGrUrUrGrArArArArGrArGrCrArUrCrGrGrUrCrUrGr | |
| | | | ArArGrGrArUrGrCrArCrUrCrCrGrGrGrArUrArGrGrGrCrA | |
| | | | rGrUrCrCrCrGrGrCrUrCrUrUrGrCrUrArArGrUrCrCrUrUr | |
| | | | CrArGrCrArArGrUrCrGrArArArGrArCrArCrGrArUrGrUrG | |
| | | | rArGrCrCrUrArUrUrUrU | |
| IID25_S | IID25_P | V129 | rGrGrArCrCrUrCrCrArGrGrUrGrCrArGrArArGrUrGrUrUr | 634 |
| yn_gRN | T_10_C1 | | UrCrArArUrCrArArCrCrGrArGrArArArUrCrGrGrUrCrCrG | |
| A024 | QTNF12 | | rGrUrUrGrArArArArGrArGrCrArUrCrGrGrUrCrUrGrArAr | |
| | | | GrGrArUrGrCrArCrUrCrCrGrGrGrArUrArGrGrGrCrArGrU | |
| | | | rCrCrCrGrGrCrUrCrUrUrGrCrUrArArGrUrCrCrUrUrCrAr | |
| | | | GrCrArArGrUrCrGrArArArGrArCrArCrGrArUrGrUrGrArG | |
| | | | rCrCrUrArUrUrUrU | |
| IID25_S | IID25_P | V130 | rGrGrArCrCrUrCrCrArGrGrUrGrCrArGrArArGrUrGrUrUr | 635 |
| yn_gRN | T_10_C1 | | UrCrArArUrCrArArCrCrGrArUrArGrArArArUrArUrCrGrG | |
| A025 | QTNF12 | | rUrCrCrGrGrUrUrGrArArArArGrArGrCrArUrCrGrGrCrUr | |
| | | | GrArArGrGrArUrGrCrArCrUrCrCrGrGrGrArUrArGrGrGrC | |
| | | | rArGrUrCrCrCrGrGrCrUrCrUrUrGrCrUrArArGrUrCrCrUr | |
| | | | UrCrArGrCrArArGrUrCrGrArArArGrArCrArCrGrArUrGrU | |
| | | | rGrArGrCrCrUrArUrUrUrU | |
| IID25_S | IID25_P | V134 | rGrGrArCrCrUrCrCrArGrGrUrGrCrArGrArArGrUrGrUrUr | 636 |
| yn_gRN | T_10_C1 | | UrCrArArUrCrArArCrCrGrArUrArGrArArArUrArUrCrGrG | |
| A026 | QTNF12 | | rUrCrCrGrGrUrUrGrArArArArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrCrGrArArArGrCrArCrGrArUrGrUrG | |
| | | | rArGrCrCrUrArUrUrUrU | |
| IID25_S | IID25_P | V117 | rCrUrGrUrGrArArGrArGrCrCrArGrUrGrGrCrCrArGrCrUr | 637 |
| yn_gRN | T_15_CP | | UrCrArArUrCrArArCrCrGrArUrArGrArArArUrArUrCrGrG | |
| A027 | TP | | rUrCrCrGrGrUrUrGrArArGrArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | IID25_P | V118 | rCrUrGrUrGrArArGrArGrCrCrArGrUrGrGrCrCrArGrUrCr | 638 |
| yn_gRN | T_15_CP | | UrCrArArUrCrArArCrCrGrArUrArGrArArArUrArUrCrGrG | |
| A028 | TP | | rUrCrCrGrGrUrUrGrArGrArArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | IID25_P | V123 | rCrUrGrUrGrArArGrArGrCrCrArGrUrGrGrCrCrArGrUrUr | 639 |
| yn_gRN | T_15_CP | | UrCrGrArUrCrArArCrCrGrArUrArGrArArArUrArUrCrGrG | |
| A029 | TP | | rUrCrCrGrGrUrCrGrArArArArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | IID25_P | V126 | rCrUrGrUrGrArArGrArGrCrCrArGrUrGrGrCrCrArGrUrUr | 640 |
| yn_gRN | T_15_CP | | UrCrArArUrCrArGrCrCrGrArUrArGrArArArUrArUrCrGrG | |
| A030 | TP | | rCrCrCrGrGrUrUrGrArArArArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | IID25_P | V127 | rCrUrGrUrGrArArGrArGrCrCrArGrUrGrGrCrCrArGrUrUr | 641 |
| yn_gRN | T_15_CP | | UrCrArArUrCrArCrCrCrGrArUrArGrArArArUrArUrCrGrG | |
| A031 | TP | | rGrCrCrGrGrUrUrGrArArArArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | IID25_P | V128 | rCrUrGrUrGrArArGrArGrCrCrArGrUrGrGrCrCrArGrUrUr | 642 |
| yn_gRN | T_15_CP | | UrCrArArUrCrArArCrCrGrArUrGrArArArArUrCrGrGrUrC | |
| A032 | TP | | rCrGrGrUrUrGrArArArArGrArGrCrArUrCrGrGrUrCrUrGr | |
| | | | ArArGrGrArUrGrCrArCrUrCrCrGrGrGrArUrArGrGrGrCrA | |
| | | | rGrUrCrCrCrGrGrCrUrCrUrUrGrCrUrArArGrUrCrCrUrUr | |
| | | | CrArGrCrArArGrUrCrGrArArArGrArCrArCrGrArUrGrUrG | |
| | | | rArGrCrCrUrArUrUrUrU | |
| IID25_S | IID25_P | V129 | rCrUrGrUrGrArArGrArGrCrCrArGrUrGrGrCrCrArGrUrUr | 643 |
| yn_gRN | T_15_CP | | UrCrArArUrCrArArCrCrGrArGrArArArUrCrGrGrUrCrCrG | |
| A033 | TP | | rGrUrUrGrArArArArGrArGrCrArUrCrGrGrUrCrUrGrArAr | |
| | | | GrGrArUrGrCrArCrUrCrCrGrGrGrArUrArGrGrGrCrArGrU | |
| | | | rCrCrCrGrGrCrUrCrUrUrGrCrUrArArGrUrCrCrUrUrCrAr | |
| | | | GrCrArArGrUrCrGrArArArGrArCrArCrGrArUrGrUrGrArG | |
| | | | rCrCrUrArUrUrUrU | |
| IID25_S | IID25_P | V130 | rCrUrGrUrGrArArGrArGrCrCrArGrUrGrGrCrCrArGrUrUr | 644 |
| yn_gRN | T_15_CP | | UrCrArArUrCrArArCrCrGrArUrArGrArArArUrArUrCrGrG | |
| A034 | TP | | rUrCrCrGrGrUrUrGrArArArArGrArGrCrArUrCrGrGrCrUr | |
| | | | GrArArGrGrArUrGrCrArCrUrCrCrGrGrGrArUrArGrGrGrC | |
| | | | rArGrUrCrCrCrGrGrCrUrCrUrUrGrCrUrArArGrUrCrCrUr | |
| | | | UrCrArGrCrArArGrUrCrGrArArArGrArCrArCrGrArUrGrU | |
| | | | rGrArGrCrCrUrArUrUrUrU | |
| IID25_S | IID25_P | V134 | rCrUrGrUrGrArArGrArGrCrCrArGrUrGrGrCrCrArGrUrUr | 645 |
| yn_gRN | T_15_CP | | UrCrArArUrCrArArCrCrGrArUrArGrArArArUrArUrCrGrG | |
| A035 | TP | | rUrCrCrGrGrUrUrGrArArArArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrCrGrArArArGrCrArCrGrArUrGrUrG | |
| | | | rArGrCrCrUrArUrUrUrU | |
| IID25_S | IID25_P | V149 | rGrArCrUrUrGrGrGrUrGrArGrCrGrArUrGrGrArCrGrCrCr | 646 |
| yn_gRN | T_5_TT | | UrCrArArUrCrArArCrCrGrArUrArGrArArArUrArUrCrGrG | |
| A036 | LL10 | | rUrCrCrGrGrUrUrGrArGrGrArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | IID25_P | V150 | rGrArCrUrUrGrGrGrUrGrArGrCrGrArUrGrGrArCrGrCrCr | 647 |
| yn_gRN | T_5_TT | | UrCrGrArUrCrArArCrCrGrArUrArGrArArArUrArUrCrGrG | |
| A037 | LL10 | | rUrCrCrGrGrUrCrGrArGrGrArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | IID25_P | V151 | rGrArCrUrUrGrGrGrUrGrArGrCrGrArUrGrGrArCrGrUrCr | 648 |
| yn_gRN | T_5_TT | | UrCrArArUrCrArArCrCrGrArUrArGrArArArUrArUrCrGrG | |
| A038 | LL10 | | rUrCrCrGrGrUrUrGrArGrArArGrArGrCrArUrCrGrGrCrUr | |
| | | | GrArArGrGrArUrGrCrArCrUrCrCrGrGrGrArUrArGrGrGrC | |
| | | | rArGrUrCrCrCrGrGrCrUrCrUrUrGrCrUrArArGrUrCrCrUr | |
| | | | UrCrArGrCrArArGrCrGrArArArGrCrArCrGrArUrGrUrGrA | |
| | | | rGrCrCrUrArUrUrUrU | |
| IID25_S | IID25_P | V152 | rGrArCrUrUrGrGrGrUrGrArGrCrGrArUrGrGrArCrGrUrCr | 649 |
| yn_gRN | T_5_TT | | UrCrArArUrCrArArCrCrGrArGrArArArUrCrGrGrUrCrCrG | |
| A039 | LL10 | | rGrUrUrGrArArArArGrArGrCrArUrCrGrGrCrUrGrArArGr | |
| | | | GrArUrGrCrArCrUrCrCrGrGrGrArUrArGrGrGrCrArGrUrC | |
| | | | rCrCrGrGrCrUrCrUrUrGrCrUrArArGrUrCrCrUrUrCrArGr | |
| | | | CrArArGrCrGrArArArGrCrArCrGrArUrGrUrGrArGrCrCrU | |
| | | | rArUrUrUrU | |
| IID25_S | IID25_P | V149 | rGrGrArCrCrUrCrCrArGrGrUrGrCrArGrArArGrUrGrCrCr | 650 |
| yn_gRN | T_10_C1 | | UrCrArArUrCrArArCrCrGrArUrArGrArArArUrArUrCrGrG | |
| A040 | QTNF12 | | rUrCrCrGrGrUrUrGrArGrGrArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | IID25_P | V150 | rGrGrArCrCrUrCrCrArGrGrUrGrCrArGrArArGrUrGrCrCr | 651 |
| yn_gRN | T_10_C1 | | UrCrGrArUrCrArArCrCrGrArUrArGrArArArUrArUrCrGrG | |
| A041 | QTNF12 | | rUrCrCrGrGrUrCrGrArGrGrArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | IID25_P | V151 | rGrGrArCrCrUrCrCrArGrGrUrGrCrArGrArArGrUrGrUrCr | 652 |
| yn_gRN | T_10_C1 | | UrCrArArUrCrArArCrCrGrArUrArGrArArArUrArUrCrGrG | |
| A042 | QTNF12 | | rUrCrCrGrGrUrUrGrArGrArArGrArGrCrArUrCrGrGrCrUr | |
| | | | GrArArGrGrArUrGrCrArCrUrCrCrGrGrGrArUrArGrGrGrC | |
| | | | rArGrUrCrCrCrGrGrCrUrCrUrUrGrCrUrArArGrUrCrCrUr | |
| | | | UrCrArGrCrArArGrCrGrArArArGrCrArCrGrArUrGrUrGrA | |
| | | | rGrCrCrUrArUrUrUrU | |
| IID25_S | IID25_P | V152 | rGrGrArCrCrUrCrCrArGrGrUrGrCrArGrArArGrUrGrUrCr | 653 |
| yn_gRN | T_10_C1 | | UrCrArArUrCrArArCrCrGrArGrArArArUrCrGrGrUrCrCrG | |
| A043 | QTNF12 | | rGrUrUrGrArArArArGrArGrCrArUrCrGrGrCrUrGrArArGr | |
| | | | GrArUrGrCrArCrUrCrCrGrGrGrArUrArGrGrGrCrArGrUrC | |
| | | | rCrCrGrGrCrUrCrUrUrGrCrUrArArGrUrCrCrUrUrCrArGr | |
| | | | CrArArGrCrGrArArArGrCrArCrGrArUrGrUrGrArGrCrCrU | |
| | | | rArUrUrUrU | |
| IID25_S | IID25_P | V149 | rCrUrGrUrGrArArGrArGrCrCrArGrUrGrGrCrCrArGrCrCr | 654 |
| yn_gRN | T_15_CP | | UrCrArArUrCrArArCrCrGrArUrArGrArArArUrArUrCrGrG | |
| A044 | TP | | rUrCrCrGrGrUrUrGrArGrGrArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | IID25_P | V150 | rCrUrGrUrGrArArGrArGrCrCrArGrUrGrGrCrCrArGrCrCr | 655 |
| yn_gRN | T_15_CP | | UrCrGrArUrCrArArCrCrGrArUrArGrArArArUrArUrCrGrG | |
| A045 | TP | | rUrCrCrGrGrUrCrGrArGrGrArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | IID25_P | V151 | rCrUrGrUrGrArArGrArGrCrCrArGrUrGrGrCrCrArGrUrCr | 656 |
| yn_gRN | T_15_CP | | UrCrArArUrCrArArCrCrGrArUrArGrArArArUrArUrCrGrG | |
| A046 | TP | | rUrCrCrGrGrUrUrGrArGrArArGrArGrCrArUrCrGrGrCrUr | |
| | | | GrArArGrGrArUrGrCrArCrUrCrCrGrGrGrArUrArGrGrGrC | |
| | | | rArGrUrCrCrCrGrGrCrUrCrUrUrGrCrUrArArGrUrCrCrUr | |
| | | | UrCrArGrCrArArGrCrGrArArArGrCrArCrGrArUrGrUrGrA | |
| | | | rGrCrCrUrArUrUrUrU | |
| IID25_S | IID25_P | V152 | rCrUrGrUrGrArArGrArGrCrCrArGrUrGrGrCrCrArGrUrCr | 657 |
| yn_gRN | T_15_CP | | UrCrArArUrCrArArCrCrGrArGrArArArUrCrGrGrUrCrCrG | |
| A047 | TP | | rGrUrUrGrArArArArGrArGrCrArUrCrGrGrCrUrGrArArGr | |
| | | | GrArUrGrCrArCrUrCrCrGrGrGrArUrArGrGrGrCrArGrUrC | |
| | | | rCrCrGrGrCrUrCrUrUrGrCrUrArArGrUrCrCrUrUrCrArGr | |
| | | | CrArArGrCrGrArArArGrCrArCrGrArUrGrUrGrArGrCrCrU | |
| | | | rArUrUrUrU | |
| IID25_S | TTR1 | v70 | rArGrCrCrArCrGrUrCrUrArCrArGrCrArGrGrGrCrGrUrUr | 658 |
| yn_gRN | | | UrCrArArUrCrArArCrCrGrArUrArGrArArArUrArUrCrGrG | |
| A048 | | | rUrCrCrGrGrUrUrGrArArArArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | TTR6 | v70 | rArCrArGrCrArGrGrGrCrUrGrCrCrUrCrGrGrArCrGrUrUr | 659 |
| yn_gRN | | | UrCrArArUrCrArArCrCrGrArUrArGrArArArUrArUrCrGrG | |
| A049 | | | rUrCrCrGrGrUrUrGrArArArArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | TTR7 | v70 | rGrArGrGrGrArUrCrCrUrGrGrGrArGrCrCrCrUrUrGrUrUr | 660 |
| yn_gRN | | | UrCrArArUrCrArArCrCrGrArUrArGrArArArUrArUrCrGrG | |
| A050 | | | rUrCrCrGrGrUrUrGrArArArArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | TTR1 | V118 | rArGrCrCrArCrGrUrCrUrArCrArGrCrArGrGrGrCrGrUrCr | 661 |
| yn_gRN | | | UrCrArArUrCrArArCrCrGrArUrArGrArArArUrArUrCrGrG | |
| A051 | | | rUrCrCrGrGrUrUrGrArGrArArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | TTR6 | V118 | rArCrArGrCrArGrGrGrCrUrGrCrCrUrCrGrGrArCrGrUrCr | 662 |
| yn_gRN | | | UrCrArArUrCrArArCrCrGrArUrArGrArArArUrArUrCrGrG | |
| A052 | | | rUrCrCrGrGrUrUrGrArGrArArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | TTR7 | V118 | rGrArGrGrGrArUrCrCrUrGrGrGrArGrCrCrCrUrUrGrUrCr | 663 |
| yn_gRN | | | UrCrArArUrCrArArCrCrGrArUrArGrArArArUrArUrCrGrG | |
| A053 | | | rUrCrCrGrGrUrUrGrArGrArArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | TTR1 + 4 | v70 | rUrUrArCrArGrCrCrArCrGrUrCrUrArCrArGrCrArGrGrGr | 664 |
| yn_gRN | | | CrGrUrUrUrCrArArUrCrArArCrCrGrArUrArGrArArArUrA | |
| A054 | | | rUrCrGrGrUrCrCrGrGrUrUrGrArArArArGrArGrCrArUrCr | |
| | | | GrGrUrCrUrGrArArGrGrArUrGrCrArCrUrCrCrGrGrGrArU | |
| | | | rArGrGrGrCrArGrUrCrCrCrGrGrCrUrCrUrUrGrCrUrArAr | |
| | | | GrUrCrCrUrUrCrArGrCrArArGrUrCrGrArArArGrArCrArC | |
| | | | rGrArUrGrUrGrArGrCrCrUrArUrUrUrU | |
| IID25_S | TTR6 + 4 | v70 | rGrUrCrUrArCrArGrCrArGrGrGrCrUrGrCrCrUrCrGrGrAr | 665 |
| yn_gRN | | | CrGrUrUrUrCrArArUrCrArArCrCrGrArUrArGrArArArUrA | |
| A055 | | | rUrCrGrGrUrCrCrGrGrUrUrGrArArArArGrArGrCrArUrCr | |
| | | | GrGrUrCrUrGrArArGrGrArUrGrCrArCrUrCrCrGrGrGrArU | |
| | | | rArGrGrGrCrArGrUrCrCrCrGrGrCrUrCrUrUrGrCrUrArAr | |
| | | | GrUrCrCrUrUrCrArGrCrArArGrUrCrGrArArArGrArCrArC | |
| | | | rGrArUrGrUrGrArGrCrCrUrArUrUrUrU | |
| IID25_S | TTR7 + 4 | v70 | rCrUrCrUrGrArGrGrGrArUrCrCrUrGrGrGrArGrCrCrCrUr | 666 |
| yn_gRN | | | UrGrUrUrUrCrArArUrCrArArCrCrGrArUrArGrArArArUrA | |
| A056 | | | rUrCrGrGrUrCrCrGrGrUrUrGrArArArArGrArGrCrArUrCr | |
| | | | GrGrUrCrUrGrArArGrGrArUrGrCrArCrUrCrCrGrGrGrArU | |
| | | | rArGrGrGrCrArGrUrCrCrCrGrGrCrUrCrUrUrGrCrUrArAr | |
| | | | GrUrCrCrUrUrCrArGrCrArArGrUrCrGrArArArGrArCrArC | |
| | | | rGrArUrGrUrGrArGrCrCrUrArUrUrUrU | |
| IID25_S | TTR1 +4 | V118 | rUrUrArCrArGrCrCrArCrGrUrCrUrArCrArGrCrArGrGrGr | 667 |
| yn_gRN | | | CrGrUrCrUrCrArArUrCrArArCrCrGrArUrArGrArArArUrA | |
| A057 | | | rUrCrGrGrUrCrCrGrGrUrUrGrArGrArArGrArGrCrArUrCr | |
| | | | GrGrUrCrUrGrArArGrGrArUrGrCrArCrUrCrCrGrGrGrArU | |
| | | | rArGrGrGrCrArGrUrCrCrCrGrGrCrUrCrUrUrGrCrUrArAr | |
| | | | GrUrCrCrUrUrCrArGrCrArArGrUrCrGrArArArGrArCrArC | |
| | | | rGrArUrGrUrGrArGrCrCrUrArUrUrUrU | |
| IID25_S | TTR6 + 4 | V118 | rGrUrCrUrArCrArGrCrArGrGrGrCrUrGrCrCrUrCrGrGrAr | 668 |
| yn_gRN | | | CrGrUrCrUrCrArArUrCrArArCrCrGrArUrArGrArArArUrA | |
| A058 | | | rUrCrGrGrUrCrCrGrGrUrUrGrArGrArArGrArGrCrArUrCr | |
| | | | GrGrUrCrUrGrArArGrGrArUrGrCrArCrUrCrCrGrGrGrArU | |
| | | | rArGrGrGrCrArGrUrCrCrCrGrGrCrUrCrUrUrGrCrUrArAr | |
| | | | GrUrCrCrUrUrCrArGrCrArArGrUrCrGrArArArGrArCrArC | |
| | | | rGrArUrGrUrGrArGrCrCrUrArUrUrUrU | |
| IID25_S | TTR7 + 4 | V118 | rCrUrCrUrGrArGrGrGrArUrCrCrUrGrGrGrArGrCrCrCrUr | 669 |
| yn_gRN | | | UrGrUrCrUrCrArArUrCrArArCrCrGrArUrArGrArArArUrA | |
| A059 | | | rUrCrGrGrUrCrCrGrGrUrUrGrArGrArArGrArGrCrArUrCr | |
| | | | GrGrUrCrUrGrArArGrGrArUrGrCrArCrUrCrCrGrGrGrArU | |
| | | | rArGrGrGrCrArGrUrCrCrCrGrGrCrUrCrUrUrGrCrUrArAr | |
| | | | GrUrCrCrUrUrCrArGrCrArArGrUrCrGrArArArGrArCrArC | |
| | | | rGrArUrGrUrGrArGrCrCrUrArUrUrUrU | |
| IID25_S | Tet | v70 | rCrUrCrCrCrUrArUrCrArGrUrGrArUrArGrArGrArGrUrUr | 670 |
| yn_gRN | reporter | | UrCrArArUrCrArArCrCrGrArUrArGrArArArUrArUrCrGrG | |
| A060 | | | rUrCrCrGrGrUrUrGrArArArArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | Tet | V118 | rCrUrCrCrCrUrArUrCrArGrUrGrArUrArGrArGrArGrUrCr | 671 |
| yn_gRN | reporter | | UrCrArArUrCrArArCrCrGrArUrArGrArArArUrArUrCrGrG | |
| A061 | | | rUrCrCrGrGrUrUrGrArGrArArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | TTR1 | V151 | rArGrCrCrArCrGrUrCrUrArCrArGrCrArGrGrGrCrGrUrCr | 672 |
| yn_gRN | | | UrCrArArUrCrArArCrCrGrArUrArGrArArArUrArUrCrGrG | |
| A062 | | | rUrCrCrGrGrUrUrGrArGrArArGrArGrCrArUrCrGrGrCrUr | |
| | | | GrArArGrGrArUrGrCrArCrUrCrCrGrGrGrArUrArGrGrGrC | |
| | | | rArGrUrCrCrCrGrGrCrUrCrUrUrGrCrUrArArGrUrCrCrUr | |
| | | | UrCrArGrCrArArGrCrGrArArArGrCrArCrGrArUrGrUrGrA | |
| | | | rGrCrCrUrArUrUrUrU | |
| IID25_S | TTR6 | V151 | rArCrArGrCrArGrGrGrCrUrGrCrCrUrCrGrGrArCrGrUrCr | 673 |
| yn_gRN | | | UrCrArArUrCrArArCrCrGrArUrArGrArArArUrArUrCrGrG | |
| A063 | | | rUrCrCrGrGrUrUrGrArGrArArGrArGrCrArUrCrGrGrCrUr | |
| | | | GrArArGrGrArUrGrCrArCrUrCrCrGrGrGrArUrArGrGrGrC | |
| | | | rArGrUrCrCrCrGrGrCrUrCrUrUrGrCrUrArArGrUrCrCrUr | |
| | | | UrCrArGrCrArArGrCrGrArArArGrCrArCrGrArUrGrUrGrA | |
| | | | rGrCrCrUrArUrUrUrU | |
| IID25_S | TTR7 | V151 | rGrArGrGrGrArUrCrCrUrGrGrGrArGrCrCrCrUrUrGrUrCr | 674 |
| yn_gRN | | | UrCrArArUrCrArArCrCrGrArUrArGrArArArUrArUrCrGrG | |
| A064 | | | rUrCrCrGrGrUrUrGrArGrArArGrArGrCrArUrCrGrGrCrUr | |
| | | | GrArArGrGrArUrGrCrArCrUrCrCrGrGrGrArUrArGrGrGrC | |
| | | | rArGrUrCrCrCrGrGrCrUrCrUrUrGrCrUrArArGrUrCrCrUr | |
| | | | UrCrArGrCrArArGrCrGrArArArGrCrArCrGrArUrGrUrGrA | |
| | | | rGrCrCrUrArUrUrUrU | |
| IID25_S | TTR1 | V151 | rArGrCrCrArCrGrUrCrUrArCrArGrCrArGrGrGrCrGrUrCr | 675 |
| yn_gRN | | | UrCrArArUrCrArArCrCrGrArUrArGrArArArUrArUrCrGrG | |
| A065 | | | rUrCrCrGrGrUrUrGrArGrArArGrArGrCrArUrCrGrGrCrUr | |
| | | | GrArArGrGrArUrGrCrArCrUrCrCrGrGrGrArUrArGrGrGrC | |
| | | | rArGrUrCrCrCrGrGrCrUrCrUrUrGrCrUrArArGrUrCrCrUr | |
| | | | UrCrArGrCrArArGrCrGrArArArGrCrArCrGrArUrGrUrGrA | |
| | | | rGrCrCrUrArUrUrUrU | |
| IID25_S | TTR6 | V151 | rArCrArGrCrArGrGrGrCrUrGrCrCrUrCrGrGrArCrGrUrCr | 676 |
| yn_gRN | | | UrCrArArUrCrArArCrCrGrArUrArGrArArArUrArUrCrGrG | |
| A066 | | | rUrCrCrGrGrUrUrGrArGrArArGrArGrCrArUrCrGrGrCrUr | |
| | | | GrArArGrGrArUrGrCrArCrUrCrCrGrGrGrArUrArGrGrGrC | |
| | | | rArGrUrCrCrCrGrGrCrUrCrUrUrGrCrUrArArGrUrCrCrUr | |
| | | | UrCrArGrCrArArGrCrGrArArArGrCrArCrGrArUrGrUrGrA | |
| | | | rGrCrCrUrArUrUrUrU | |
| IID25_S | TTR7 | V151 | rGrArGrGrGrArUrCrCrUrGrGrGrArGrCrCrCrUrUrGrUrCr | 677 |
| yn_gRN | | | UrCrArArUrCrArArCrCrGrArUrArGrArArArUrArUrCrGrG | |
| A067 | | | rUrCrCrGrGrUrUrGrArGrArArGrArGrCrArUrCrGrGrCrUr | |
| | | | GrArArGrGrArUrGrCrArCrUrCrCrGrGrGrArUrArGrGrGrC | |
| | | | rArGrUrCrCrCrGrGrCrUrCrUrUrGrCrUrArArGrUrCrCrUr | |
| | | | UrCrArGrCrArArGrCrGrArArArGrCrArCrGrArUrGrUrGrA | |
| | | | rGrCrCrUrArUrUrUrU | |
| IID25_S | TTR1 | V118 | rArGrCrCrArCrGrUrCrUrArCrArGrCrArGrGrGrCrGrUrCr | 678 |
| yn_gRN | | | UrCrArArUrCrArArCrCrGrArUrArGrArArArUrArUrCrGrG | |
| A068 | | | rUrCrCrGrGrUrUrGrArGrArArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | TTR6 | V118 | rArCrArGrCrArGrGrGrCrUrGrCrCrUrCrGrGrArCrGrUrCr | 679 |
| yn_gRN | | | UrCrArArUrCrArArCrCrGrArUrArGrArArArUrArUrCrGrG | |
| A069 | | | rUrCrCrGrGrUrUrGrArGrArArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | TTR7 | V118 | rGrArGrGrGrArUrCrCrUrGrGrGrArGrCrCrCrUrUrGrUrCr | 680 |
| yn_gRN | | | UrCrArArUrCrArArCrCrGrArUrArGrArArArUrArUrCrGrG | |
| A070 | | | rUrCrCrGrGrUrUrGrArGrArArGrArGrCrArUrCrGrGrUrCr | |
| | | | UrGrArArGrGrArUrGrCrArCrUrCrCrGrGrGrArUrArGrGrG | |
| | | | rCrArGrUrCrCrCrGrGrCrUrCrUrUrGrCrUrArArGrUrCrCr | |
| | | | UrUrCrArGrCrArArGrUrCrGrArArArGrArCrArCrGrArUrG | |
| | | | rUrGrArGrCrCrUrArUrUrUrU | |
| IID25_S | TTR1 + 4 | V118 | rUrUrArCrArGrCrCrArCrGrUrCrUrArCrArGrCrArGrGrGr | 681 |
| yn_gRN | | | CrGrUrCrUrCrArArUrCrArArCrCrGrArUrArGrArArArUrA | |
| A071 | | | rUrCrGrGrUrCrCrGrGrUrUrGrArGrArArGrArGrCrArUrCr | |
| | | | GrGrUrCrUrGrArArGrGrArUrGrCrArCrUrCrCrGrGrGrArU | |
| | | | rArGrGrGrCrArGrUrCrCrCrGrGrCrUrCrUrUrGrCrUrArAr | |
| | | | GrUrCrCrUrUrCrArGrCrArArGrUrCrGrArArArGrArCrArC | |
| | | | rGrArUrGrUrGrArGrCrCrUrArUrUrUrU | |
| IID25_S | TTR6 + 4 | V118 | rGrUrCrUrArCrArGrCrArGrGrGrCrUrGrCrCrUrCrGrGrAr | 682 |
| yn_gRN | | | CrGrUrCrUrCrArArUrCrArArCrCrGrArUrArGrArArArUrA | |
| A072 | | | rUrCrGrGrUrCrCrGrGrUrUrGrArGrArArGrArGrCrArUrCr | |
| | | | GrGrUrCrUrGrArArGrGrArUrGrCrArCrUrCrCrGrGrGrArU | |
| | | | rArGrGrGrCrArGrUrCrCrCrGrGrCrUrCrUrUrGrCrUrArAr | |
| | | | GrUrCrCrUrUrCrArGrCrArArGrUrCrGrArArArGrArCrArC | |
| | | | rGrArUrGrUrGrArGrCrCrUrArUrUrUrU | |
| IID25_S | TTR7 + 4 | V118 | rCrUrCrUrGrArGrGrGrArUrCrCrUrGrGrGrArGrCrCrCrUr | 683 |
| yn_gRN | | | UrGrUrCrUrCrArArUrCrArArCrCrGrArUrArGrArArArUrA | |
| A073 | | | rUrCrGrGrUrCrCrGrGrUrUrGrArGrArArGrArGrCrArUrCr | |
| | | | GrGrUrCrUrGrArArGrGrArUrGrCrArCrUrCrCrGrGrGrArU | |
| | | | rArGrGrGrCrArGrUrCrCrCrGrGrCrUrCrUrUrGrCrUrArAr | |
| | | | GrUrCrCrUrUrCrArGrCrArArGrUrCrGrArArArGrArCrArC | |
| | | | rGrArUrGrUrGrArGrCrCrUrArUrUrUrU | |

TABLE 7D
Exemplary modified RNA Sequences of Guides: SI# indicates the
corresponding SEQ ID NO; “m” indicates a 2′-OMe modification
on the next letter; “r” indicates that
the next letter is a ribonucleotide; “*”
indicates a phosphorothioate linkage in between the nucleotides.

| | | Scaffold | | |
|---|---|---|---|---|
| RNA ID | Target ID | ID | Modified RNA Seq 5′ | SI# |
| IID25 S | EMX1-2 | v70 | mC*mU*mC*rCrArArUrGrArCrUrArGrGrGrUrGrGrGrCrG | 684 |
| yn_gRN | | | rUrUrUrCrArArUrCrArArCrCrGrArUrArGrArArArUrArUr | |
| A001 | | | CrGrGrUrCrCrGrGrUrUrGrArArArArGrArGrCrArUrCrGrG | |
| | | | rUrCrUrGrArArGrGrArUrGrCrArCrUrCrCrGrGrGrArUrAr | |
| | | | GrGrGrCrArGrUrCrCrCrGrGrCrUrCrUrUrGrCrUrArArGrU | |
| | | | rCrCrUrUrCrArGrCrArArGrUrCrGrArArArGrArCrArCrGr | |
| | | | ArUrGrUrGrArGrCrCrUrArUrUrUrU | |
| IID25_S | EMX1-4 | v70 | mA*mU*mG*rGrGrCrUrGrGrCrUrGrArGrGrCrCrUrGrGrG | 685 |
| yn_gRN | | | rUrUrUrCrArArUrCrArArCrCrGrArUrArGrArArArUrArUr | |
| A002 | | | CrGrGrUrCrCrGrGrUrUrGrArArArArGrArGrCrArUrCrGrG | |
| | | | rUrCrUrGrArArGrGrArUrGrCrArCrUrCrCrGrGrGrArUrAr | |
| | | | GrGrGrCrArGrUrCrCrCrGrGrCrUrCrUrUrGrCrUrArArGrU | |
| | | | rCrCrUrUrCrArGrCrArArGrUrCrGrArArArGrArCrArCrGr | |
| | | | ArUrGrUrGrArGrCrCrUrArUrUrUrU | |
| IID25_S | HPRT38 | v70 | mA*mG*mA*rCrArArGrGrCrArCrUrUrGrGrUrUrGrArUr | 686 |
| yn_gRN | 07 | | GrUrUrUrCrArArUrCrArArCrCrGrArUrArGrArArArUrArU | |
| A003 | | | rCrGrGrUrCrCrGrGrUrUrGrArArArArGrArGrCrArUrCrGr | |
| | | | GrUrCrUrGrArArGrGrArUrGrCrArCrUrCrCrGrGrGrArUrA | |
| | | | rGrGrGrCrArGrUrCrCrCrGrGrCrUrCrUrUrGrCrUrArArGr | |
| | | | UrCrCrUrUrCrArGrCrArArGrUrCrGrArArArGrArCrArCrG | |
| | | | rArUrGrUrGrArGrCrCrUrArUrUrUrU | |
| IID25_S | VEGFA1 | v70 | mC*mC*mC*rArCrArCrGrCrArCrArCrArCrUrCrArCrUrGr | 687 |
| yn_gRN | | | UrUrUrCrArArUrCrArArCrCrGrArUrArGrArArArUrArUrC | |
| A004 | | | rGrGrUrCrCrGrGrUrUrGrArArArArGrArGrCrArUrCrGrGr | |
| | | | UrCrUrGrArArGrGrArUrGrCrArCrUrCrCrGrGrGrArUrArG | |
| | | | rGrGrCrArGrUrCrCrCrGrGrCrUrCrUrUrGrCrUrArArGrUr | |
| | | | CrCrUrUrCrArGrCrArArGrUrCrGrArArArGrArCrArCrGrA | |
| | | | rUrGrUrGrArGrCrCrUrArUrUrUrU | |
| IID25_S | IID25_P | v70 | mG*mA*mC*rUrUrGrGrGrUrGrArGrCrGrArUrGrGrArCr | 688 |
| yn_gRN | T_5_TT | | GrUrUrUrCrArArUrCrArArCrCrGrArUrArGrArArArUrArU | |
| A005 | LL10 | | rCrGrGrUrCrCrGrGrUrUrGrArArArArGrArGrCrArUrCrGr | |
| | | | GrUrCrUrGrArArGrGrArUrGrCrArCrUrCrCrGrGrGrArUrA | |
| | | | rGrGrGrCrArGrUrCrCrCrGrGrCrUrCrUrUrGrCrUrArArGr | |
| | | | UrCrCrUrUrCrArGrCrArArGrUrCrGrArArArGrArCrArCrG | |
| | | | rArUrGrUrGrArGrCrCrUrArUrUrUrU | |
| IID25_S | IID25_P | v70 | mG*mG*mA*rCrCrUrCrCrArGrGrUrGrCrArGrArArGrUrG | 689 |
| yn_gRN | T_10_C1 | | rUrUrUrCrArArUrCrArArCrCrGrArUrArGrArArArUrArUr | |
| A006 | QTNF12 | | CrGrGrUrCrCrGrGrUrUrGrArArArArGrArGrCrArUrCrGrG | |
| | | | rUrCrUrGrArArGrGrArUrGrCrArCrUrCrCrGrGrGrArUrAr | |
| | | | GrGrGrCrArGrUrCrCrCrGrGrCrUrCrUrUrGrCrUrArArGrU | |
| | | | rCrCrUrUrCrArGrCrArArGrUrCrGrArArArGrArCrArCrGr | |
| | | | ArUrGrUrGrArGrCrCrUrArUrUrUrU | |
| IID25_S | IID25_P | v70 | mC*mU*mG*rUrGrArArGrArGrCrCrArGrUrGrGrCrCrArG | 690 |
| yn_gRN | T_15_CP | | rUrUrUrCrArArUrCrArArCrCrGrArUrArGrArArArUrArUr | |
| A007 | TP | | CrGrGrUrCrCrGrGrUrUrGrArArArArGrArGrCrArUrCrGrG | |
| | | | rUrCrUrGrArArGrGrArUrGrCrArCrUrCrCrGrGrGrArUrAr | |
| | | | GrGrGrCrArGrUrCrCrCrGrGrCrUrCrUrUrGrCrUrArArGrU | |
| | | | rCrCrUrUrCrArGrCrArArGrUrCrGrArArArGrArCrArCrGr | |
| | | | ArUrGrUrGrArGrCrCrUrArUrUrUrU | |
| IID25_S | IID25_P | v70 | mG*mG*mA*rCrArArArGrGrUrCrCrUrCrArArArGrArCrG | 691 |
| yn_gRN | T_22_V | | rUrUrUrCrArArUrCrArArCrCrGrArUrArGrArArArUrArUr | |
| A008 | WA1 | | CrGrGrUrCrCrGrGrUrUrGrArArArArGrArGrCrArUrCrGrG | |
| | | | rUrCrUrGrArArGrGrArUrGrCrArCrUrCrCrGrGrGrArUrAr | |
| | | | GrGrGrCrArGrUrCrCrCrGrGrCrUrCrUrUrGrCrUrArArGrU | |
| | | | rCrCrUrUrCrArGrCrArArGrUrCrGrArArArGrArCrArCrGr | |
| | | | ArUrGrUrGrArGrCrCrUrArUrUrUrU | |
| IID25_S | IID25_P | V117 | mG*mA*mC*rUrUrGrGrGrUrGrArGrCrGrArUrGrGrArCr | 692 |
| yn_gRN | T_5_TT | | GrCrUrUrCrArArUrCrArArCrCrGrArUrArGrArArArUrArU | |
| A009 | LL10 | | rCrGrGrUrCrCrGrGrUrUrGrArArGrArGrArGrCrArUrCrGr | |
| | | | GrUrCrUrGrArArGrGrArUrGrCrArCrUrCrCrGrGrGrArUrA | |
| | | | rGrGrGrCrArGrUrCrCrCrGrGrCrUrCrUrUrGrCrUrArArGr | |
| | | | UrCrCrUrUrCrArGrCrArArGrUrCrGrArArArGrArCrArCrG | |
| | | | rArUrGrUrGrArGrCrCrUrArUrUrUrU | |
| IID25_S | IID25_P | V118 | mG*mA*mC*rUrUrGrGrGrUrGrArGrCrGrArUrGrGrArCr | 693 |
| yn_gRN | T_5_TT | | GrUrCrUrCrArArUrCrArArCrCrGrArUrArGrArArArUrArU | |
| A010 | LL10 | | rCrGrGrUrCrCrGrGrUrUrGrArGrArArGrArGrCrArUrCrGr | |
| | | | GrUrCrUrGrArArGrGrArUrGrCrArCrUrCrCrGrGrGrArUrA | |
| | | | rGrGrGrCrArGrUrCrCrCrGrGrCrUrCrUrUrGrCrUrArArGr | |
| | | | UrCrCrUrUrCrArGrCrArArGrUrCrGrArArArGrArCrArCrG | |
| | | | rArUrGrUrGrArGrCrCrUrArUrUrUrU | |
| IID25_S | IID25_P | V123 | mG*mA*mC*rUrUrGrGrGrUrGrArGrCrGrArUrGrGrArCr | 694 |
| yn_gRN | T_5_TT | | GrUrUrUrCrGrArUrCrArArCrCrGrArUrArGrArArArUrArU | |
| A011 | LL10 | | rCrGrGrUrCrCrGrGrUrCrGrArArArArGrArGrCrArUrCrGr | |
| | | | GrUrCrUrGrArArGrGrArUrGrCrArCrUrCrCrGrGrGrArUrA | |
| | | | rGrGrGrCrArGrUrCrCrCrGrGrCrUrCrUrUrGrCrUrArArGr | |
| | | | UrCrCrUrUrCrArGrCrArArGrUrCrGrArArArGrArCrArCrG | |
| | | | rArUrGrUrGrArGrCrCrUrArUrUrUrU | |
| IID25_S | IID25_P | V126 | mG*mA*mC*rUrUrGrGrGrUrGrArGrCrGrArUrGrGrArCr | 695 |
| yn_gRN | T_5_TT | | GrUrUrUrCrArArUrCrArGrCrCrGrArUrArGrArArArUrArU | |
| A012 | LL10 | | rCrGrGrCrCrCrGrGrUrUrGrArArArArGrArGrCrArUrCrGr | |
| | | | GrUrCrUrGrArArGrGrArUrGrCrArCrUrCrCrGrGrGrArUrA | |
| | | | rGrGrGrCrArGrUrCrCrCrGrGrCrUrCrUrUrGrCrUrArArGr | |
| | | | UrCrCrUrUrCrArGrCrArArGrUrCrGrArArArGrArCrArCrG | |
| | | | rArUrGrUrGrArGrCrCrUrArUrUrUrU | |
| IID25_S | IID25_P | V127 | mG*mA*mC*rUrUrGrGrGrUrGrArGrCrGrArUrGrGrArCr | 696 |
| yn_gRN | T_5_TT | | GrUrUrUrCrArArUrCrArCrCrCrGrArUrArGrArArArUrArU | |
| A013 | LL10 | | rCrGrGrGrCrCrGrGrUrUrGrArArArArGrArGrCrArUrCrGr | |
| | | | GrUrCrUrGrArArGrGrArUrGrCrArCrUrCrCrGrGrGrArUrA | |
| | | | rGrGrGrCrArGrUrCrCrCrGrGrCrUrCrUrUrGrCrUrArArGr | |
| | | | UrCrCrUrUrCrArGrCrArArGrUrCrGr ArArArGrArCrArCrG | |
| | | | rArUrGrUrGrArGrCrCrUrArUrUrUrU | |
| IID25_S | IID25_P | V128 | mG*mA*mC*rUrUrGrGrGrUrGrArGrCrGrArUrGrGrArCr | 697 |
| yn_gRN | T_5_TT | | GrUrUrUrCrArArUrCrArArCrCrGrArUrGrArArArArUrCrG | |
| A014 | LL10 | | rGrUrCrCrGrGrUrUrGrArArArArGrArGrCrArUrCrGrGrUr | |
| | | | CrUrGrArArGrGrArUrGrCrArCrUrCrCrGrGrGrArUrArGrG | |
| | | | rGrCrArGrUrCrCrCrGrGrCrUrCrUrUrGrCrUrArArGrUrCr | |
| | | | CrUrUrCrArGrCrArArGrUrCrGrArArArGrArCrArCrGrArU | |
| | | | rGrUrGrArGrCrCrUrArUrUrUrU | |
| IID25_S | IID25_P | V129 | mG*mA*mC*rUrUrGrGrGrUrGrArGrCrGrArUrGrGrArCr | 698 |
| yn_gRN | T_5_TT | | GrUrUrUrCrArArUrCrArArCrCrGrArGrArArArUrCrGrGrU | |
| A015 | LL10 | | rCrCrGrGrUrUrGrArArArArGrArGrCrArUrCrGrGrUrCrUr | |
| | | | GrArArGrGrArUrGrCrArCrUrCrCrGrGrGrArUrArGrGrGrC | |
| | | | rArGrUrCrCrCrGrGrCrUrCrUrUrGrCrUrArArGrUrCrCrUr | |
| | | | UrCrArGrCrArArGrUrCrGrArArArGrArCrArCrGrArUrGrU | |
| | | | rGrArGrCrCrUrArUrUrUrU | |
| IID25_S | IID25_P | V130 | mG*mA*mC*rUrUrGrGrGrUrGrArGrCrGrArUrGrGrArCr | 699 |
| yn_gRN | T_5_TT | | GrUrUrUrCrArArUrCrArArCrCrGrArUrArGrArArArUrArU | |
| A016 | LL10 | | rCrGrGrUrCrCrGrGrUrUrGrArArArArGrArGrCrArUrCrGr | |
| | | | GrCrUrGrArArGrGrArUrGrCrArCrUrCrCrGrGrGrArUrArG | |
| | | | rGrGrCrArGrUrCrCrCrGrGrCrUrCrUrUrGrCrUrArArGrUr | |
| | | | CrCrUrUrCrArGrCrArArGrUrCrGrArArArGrArCrArCrGrA | |
| | | | rUrGrUrGrArGrCrCrUrArUrUrUrU | |
| IID25_S | IID25_P | V134 | mG*mA*mC*rUrUrGrGrGrUrGrArGrCrGrArUrGrGrArCr | 700 |
| yn_gRN | T_5_TT | | GrUrUrUrCrArArUrCrArArCrCrGrArUrArGrArArArUrArU | |
| A017 | LL10 | | rCrGrGrUrCrCrGrGrUrUrGrArArArArGrArGrCrArUrCrGr | |
| | | | GrUrCrUrGrArArGrGrArUrGrCrArCrUrCrCrGrGrGrArUrA | |
| | | | rGrGrGrCrArGrUrCrCrCrGrGrCrUrCrUrUrGrCrUrArArGr | |
| | | | UrCrCrUrUrCrArGrCrArArGrCrGrArArArGrCrArCrGrArU | |
| | | | rGrUrGrArGrCrCrUrArUrUrUrU | |
| IID25_S | IID25_P | V117 | mG*mG*mA*rCrCrUrCrCrArGrGrUrGrCrArGrArArGrUrG | 701 |
| yn_gRN | T_10_C1 | | rCrUrUrCrArArUrCrArArCrCrGrArUrArGrArArArUrArUr | |
| A018 | QTNF12 | | CrGrGrUrCrCrGrGrUrUrGrArArGrArGrArGrCrArUrCrGrG | |
| | | | rUrCrUrGrArArGrGrArUrGrCrArCrUrCrCrGrGrGrArUrAr | |
| | | | GrGrGrCrArGrUrCrCrCrGrGrCrUrCrUrUrGrCrUrArArGrU | |
| | | | rCrCrUrUrCrArGrCrArArGrUrCrGrArArArGrArCrArCrGr | |
| | | | ArUrGrUrGrArGrCrCrUrArUrUrUrU | |
| IID25_S | IID25_P | V118 | mG*mG*mA*rCrCrUrCrCrArGrGrUrGrCrArGrArArGrUrG | 702 |
| yn_gRN | T_10_C1 | | rUrCrUrCrArArUrCrArArCrCrGrArUrArGrArArArUrArUr | |
| A019 | QTNF12 | | CrGrGrUrCrCrGrGrUrUrGrArGrArArGrArGrCrArUrCrGrG | |
| | | | rUrCrUrGrArArGrGrArUrGrCrArCrUrCrCrGrGrGrArUrAr | |
| | | | GrGrGrCrArGrUrCrCrCrGrGrCrUrCrUrUrGrCrUrArArGrU | |
| | | | rCrCrUrUrCrArGrCrArArGrUrCrGrArArArGrArCrArCrGr | |
| | | | ArUrGrUrGrArGrCrCrUrArUrUrUrU | |
| IID25_S | IID25_P | V123 | mG*mG*mA*rCrCrUrCrCrArGrGrUrGrCrArGrArArGrUrG | 703 |
| yn_gRN | T_10_C1 | | rUrUrUrCrGrArUrCrArArCrCrGrArUrArGrArArArUrArUr | |
| A020 | QTNF12 | | CrGrGrUrCrCrGrGrUrCrGrArArArArGrArGrCrArUrCrGrG | |
| | | | rUrCrUrGrArArGrGrArUrGrCrArCrUrCrCrGrGrGrArUrAr | |
| | | | GrGrGrCrArGrUrCrCrCrGrGrCrUrCrUrUrGrCrUrArArGrU | |
| | | | rCrCrUrUrCrArGrCrArArGrUrCrGrArArArGrArCrArCrGr | |
| | | | ArUrGrUrGrArGrCrCrUrArUrUrUrU | |
| IID25_S | IID25_P | V126 | mG*mG*mA*rCrCrUrCrCrArGrGrUrGrCrArGrArArGrUrG | 704 |
| yn_gRN | T_10_C1 | | rUrUrUrCrArArUrCrArGrCrCrGrArUrArGrArArArUrArUr | |
| A021 | QTNF12 | | CrGrGrCrCrCrGrGrUrUrGrArArArArGrArGrCrArUrCrGrG | |
| | | | rUrCrUrGrArArGrGrArUrGrCrArCrUrCrCrGrGrGrArUrAr | |
| | | | GrGrGrCrArGrUrCrCrCrGrGrCrUrCrUrUrGrCrUrArArGrU | |
| | | | rCrCrUrUrCrArGrCrArArGrUrCrGrArArArGrArCrArCrGr | |
| | | | ArUrGrUrGrArGrCrCrUrArUrUrUrU | |
| IID25_S | IID25_P | V127 | mG*mG*mA*rCrCrUrCrCrArGrGrUrGrCrArGrArArGrUrG | 705 |
| yn_gRN | T_10_C1 | | rUrUrUrCrArArUrCrArCrCrCrGrArUrArGrArArArUrArUr | |
| A022 | QTNF12 | | CrGrGrGrCrCrGrGrUrUrGrArArArArGrArGrCrArUrCrGrG | |
| | | | rUrCrUrGrArArGrGrArUrGrCrArCrUrCrCrGrGrGrArUrAr | |
| | | | GrGrGrCrArGrUrCrCrCrGrGrCrUrCrUrUrGrCrUrArArGrU | |
| | | | rCrCrUrUrCrArGrCrArArGrUrCrGrArArArGrArCrArCrGr | |
| | | | ArUrGrUrGrArGrCrCrUrArUrUrUrU | |
| IID25_S | IID25_P | V128 | mG*mG*mA*rCrCrUrCrCrArGrGrUrGrCrArGrArArGrUrG | 706 |
| yn_gRN | T_10_C1 | | rUrUrUrCrArArUrCrArArCrCrGrArUrGrArArArArUrCrGr | |
| A023 | QTNF12 | | GrUrCrCrGrGrUrUrGrArArArArGrArGrCrArUrCrGrGrUrC | |
| | | | rUrGrArArGrGrArUrGrCrArCrUrCrCrGrGrGrArUrArGrGr | |
| | | | GrCrArGrUrCrCrCrGrGrCrUrCrUrUrGrCrUrArArGrUrCrC | |
| | | | rUrUrCrArGrCrArArGrUrCrGrArArArGrArCrArCrGrArUr | |
| | | | GrUrGrArGrCrCrUrArUrUrUrU | |
| IID25_S | IID25_P | V129 | mG*mG*mA*rCrCrUrCrCrArGrGrUrGrCrArGrArArGrUrG | 707 |
| yn_gRN | T_10_C1 | | rUrUrUrCrArArUrCrArArCrCrGrArGrArArArUrCrGrGrUr | |
| A024 | QTNF12 | | CrCrGrGrUrUrGrArArArArGrArGrCrArUrCrGrGrUrCrUrG | |
| | | | rArArGrGrArUrGrCrArCrUrCrCrGrGrGrArUrArGrGrGrCr | |
| | | | ArGrUrCrCrCrGrGrCrUrCrUrUrGrCrUrArArGrUrCrCrUrU | |
| | | | rCrArGrCrArArGrUrCrGrArArArGrArCrArCrGrArUrGrUr | |
| | | | GrArGrCrCrUrArUrUrUrU | |
| IID25_S | IID25_P | V130 | mG*mG*mA*rCrCrUrCrCrArGrGrUrGrCrArGrArArGrUrG | 708 |
| yn_gRN | T_10_C1 | | rUrUrUrCrArArUrCrArArCrCrGrArUrArGrArArArUrArUr | |
| A025 | QTNF12 | | CrGrGrUrCrCrGrGrUrUrGrArArArArGrArGrCrArUrCrGrG | |
| | | | rCrUrGrArArGrGrArUrGrCrArCrUrCrCrGrGrGrArUrArGr | |
| | | | GrGrCrArGrUrCrCrCrGrGrCrUrCrUrUrGrCrUrArArGrUrC | |
| | | | rCrUrUrCrArGrCrArArGrUrCrGrArArArGrArCrArCrGrAr | |
| | | | UrGrUrGrArGrCrCrUrArUrUrUrU | |
| IID25_S | IID25_P | V134 | mG*mG*mA*rCrCrUrCrCrArGrGrUrGrCrArGrArArGrUrG | 709 |
| yn_gRN | T_10_C1 | | rUrUrUrCrArArUrCrArArCrCrGrArUrArGrArArArUrArUr | |
| A026 | QTNF12 | | CrGrGrUrCrCrGrGrUrUrGrArArArArGrArGrCrArUrCrGrG | |
| | | | rUrCrUrGrArArGrGrArUrGrCrArCrUrCrCrGrGrGrArUrAr | |
| | | | GrGrGrCrArGrUrCrCrCrGrGrCrUrCrUrUrGrCrUrArArGrU | |
| | | | rCrCrUrUrCrArGrCrArArGrCrGrArArArGrCrArCrGrArUr | |
| | | | GrUrGrArGrCrCrUrArUrUrUrU | |
| IID25_S | IID25_P | V117 | mC*mU*mG*rUrGrArArGrArGrCrCrArGrUrGrGrCrCrArG | 710 |
| yn_gRN | T_15_CP | | rCrUrUrCrArArUrCrArArCrCrGrArUrArGrArArArUrArUr | |
| A027 | TP | | CrGrGrUrCrCrGrGrUrUrGrArArGrArGrArGrCrArUrCrGrG | |
| | | | rUrCrUrGrArArGrGrArUrGrCrArCrUrCrCrGrGrGrArUrAr | |
| | | | GrGrGrCrArGrUrCrCrCrGrGrCrUrCrUrUrGrCrUrArArGrU | |
| | | | rCrCrUrUrCrArGrCrArArGrUrCrGrArArArGrArCrArCrGr | |
| | | | ArUrGrUrGrArGrCrCrUrArUrUrUrU | |
| IID25_S | IID25_P | V118 | mC*mU*mG*rUrGrArArGrArGrCrCrArGrUrGrGrCrCrArG | 711 |
| yn_gRN | T_15_CP | | rUrCrUrCrArArUrCrArArCrCrGrArUrArGrArArArUrArUr | |
| A028 | TP | | CrGrGrUrCrCrGrGrUrUrGrArGrArArGrArGrCrArUrCrGrG | |
| | | | rUrCrUrGrArArGrGrArUrGrCrArCrUrCrCrGrGrGrArUrAr | |
| | | | GrGrGrCrArGrUrCrCrCrGrGrCrUrCrUrUrGrCrUrArArGrU | |
| | | | rCrCrUrUrCrArGrCrArArGrUrCrGrArArArGrArCrArCrGr | |
| | | | ArUrGrUrGrArGrCrCrUrArUrUrUrU | |
| IID25_S | IID25_P | V123 | mC*mU*mG*rUrGrArArGrArGrCrCrArGrUrGrGrCrCrArG | 712 |
| yn_gRN | T_15_CP | | rUrUrUrCrGrArUrCrArArCrCrGrArUrArGrArArArUrArUr | |
| A029 | TP | | CrGrGrUrCrCrGrGrUrCrGrArArArArGrArGrCrArUrCrGrG | |
| | | | rUrCrUrGrArArGrGrArUrGrCrArCrUrCrCrGrGrGrArUrAr | |
| | | | GrGrGrCrArGrUrCrCrCrGrGrCrUrCrUrUrGrCrUrArArGrU | |
| | | | rCrCrUrUrCrArGrCrArArGrUrCrGrArArArGrArCrArCrGr | |
| | | | ArUrGrUrGrArGrCrCrUrArUrUrUrU | |
| IID25_S | IID25_P | V126 | mC*mU*mG*rUrGrArArGrArGrCrCrArGrUrGrGrCrCrArG | 713 |
| yn_gRN | T_15_CP | | rUrUrUrCrArArUrCrArGrCrCrGrArUrArGrArArArUrArUr | |
| A030 | TP | | CrGrGrCrCrCrGrGrUrUrGrArArArArGrArGrCrArUrCrGrG | |
| | | | rUrCrUrGrArArGrGrArUrGrCrArCrUrCrCrGrGrGrArUrAr | |
| | | | GrGrGrCrArGrUrCrCrCrGrGrCrUrCrUrUrGrCrUrArArGrU | |
| | | | rCrCrUrUrCrArGrCrArArGrUrCrGrArArArGrArCrArCrGr | |
| | | | ArUrGrUrGrArGrCrCrUrArUrUrUrU | |
| IID25_S | IID25_P | V127 | mC*mU*mG*rUrGrArArGrArGrCrCrArGrUrGrGrCrCrArG | 714 |
| yn_gRN | T_15_CP | | rUrUrUrCrArArUrCrArCrCrCrGrArUrArGrArArArUrArUr | |
| A031 | TP | | CrGrGrGrCrCrGrGrUrUrGrArArArArGrArGrCrArUrCrGrG | |
| | | | rUrCrUrGrArArGrGrArUrGrCrArCrUrCrCrGrGrGrArUrAr | |
| | | | GrGrGrCrArGrUrCrCrCrGrGrCrUrCrUrUrGrCrUrArArGrU | |
| | | | rCrCrUrUrCrArGrCrArArGrUrCrGrArArArGrArCrArCrGr | |
| | | | ArUrGrUrGrArGrCrCrUrArUrUrUrU | |
| IID25_S | IID25_P | V128 | mC*mU*mG*rUrGrArArGrArGrCrCrArGrUrGrGrCrCrArG | 715 |
| yn_gRN | T_15_CP | | rUrUrUrCrArArUrCrArArCrCrGrArUrGrArArArArUrCrGr | |
| A032 | TP | | GrUrCrCrGrGrUrUrGrArArArArGrArGrCrArUrCrGrGrUrC | |
| | | | rUrGrArArGrGrArUrGrCrArCrUrCrCrGrGrGrArUrArGrGr | |
| | | | GrCrArGrUrCrCrCrGrGrCrUrCrUrUrGrCrUrArArGrUrCrC | |
| | | | rUrUrCrArGrCrArArGrUrCrGrArArArGrArCrArCrGrArUr | |
| | | | GrUrGrArGrCrCrUrArUrUrUrU | |
| IID25_S | IID25_P | V129 | mC*mU*mG*rUrGrArArGrArGrCrCrArGrUrGrGrCrCrArG | 716 |
| yn_gRN | T_15_CP | | rUrUrUrCrArArUrCrArArCrCrGrArGrArArArUrCrGrGrUr | |
| A033 | TP | | CrCrGrGrUrUrGrArArArArGrArGrCrArUrCrGrGrUrCrUrG | |
| | | | rArArGrGrArUrGrCrArCrUrCrCrGrGrGrArUrArGrGrGrCr | |
| | | | ArGrUrCrCrCrGrGrCrUrCrUrUrGrCrUrArArGrUrCrCrUrU | |
| | | | rCrArGrCrArArGrUrCrGrArArArGrArCrArCrGrArUrGrUr | |
| | | | GrArGrCrCrUrArUrUrUrU | |
| IID25_S | IID25_P | V130 | mC*mU*mG*rUrGrArArGrArGrCrCrArGrUrGrGrCrCrArG | 717 |
| yn_gRN | T_15_CP | | rUrUrUrCrArArUrCrArArCrCrGrArUrArGrArArArUrArUr | |
| A034 | TP | | CrGrGrUrCrCrGrGrUrUrGrArArArArGrArGrCrArUrCrGrG | |
| | | | rCrUrGrArArGrGrArUrGrCrArCrUrCrCrGrGrGrArUrArGr | |
| | | | GrGrCrArGrUrCrCrCrGrGrCrUrCrUrUrGrCrUrArArGrUrC | |
| | | | rCrUrUrCrArGrCrArArGrUrCrGrArArArGrArCrArCrGrAr | |
| | | | UrGrUrGrArGrCrCrUrArUrUrUrU | |
| IID25_S | IID25_P | V134 | mC*mU*mG*rUrGrArArGrArGrCrCrArGrUrGrGrCrCrArG | 718 |
| yn_gRN | T_15_CP | | rUrUrUrCrArArUrCrArArCrCrGrArUrArGrArArArUrArUr | |
| A035 | TP | | CrGrGrUrCrCrGrGrUrUrGrArArArArGrArGrCrArUrCrGrG | |
| | | | rUrCrUrGrArArGrGrArUrGrCrArCrUrCrCrGrGrGrArUrAr | |
| | | | GrGrGrCrArGrUrCrCrCrGrGrCrUrCrUrUrGrCrUrArArGrU | |
| | | | rCrCrUrUrCrArGrCrArArGrCrGrArArArGrCrArCrGrArUr | |
| | | | GrUrGrArGrCrCrUrArUrUrUrU | |
| IID25_S | IID25_P | V149 | mG*mA*mC*rUrUrGrGrGrUrGrArGrCrGrArUrGrGrArCr | 719 |
| yn_gRN | T_5_TT | | GrCrCrUrCrArArUrCrArArCrCrGrArUrArGrArArArUrArU | |
| A036 | LL10 | | rCrGrGrUrCrCrGrGrUrUrGrArGrGrArGrArGrCrArUrCrGr | |
| | | | GrUrCrUrGrArArGrGrArUrGrCrArCrUrCrCrGrGrGrArUrA | |
| | | | rGrGrGrCrArGrUrCrCrCrGrGrCrUrCrUrUrGrCrUrArArGr | |
| | | | UrCrCrUrUrCrArGrCrArArGrUrCrGrArArArGrArCrArCrG | |
| | | | rArUrGrUrGrArGrCrCrUrArUrUrUrU | |
| IID25_S | IID25_P | V150 | mG*mA*mC*rUrUrGrGrGrUrGrArGrCrGrArUrGrGrArCr | 720 |
| yn_gRN | T_5_TT | | GrCrCrUrCrGrArUrCrArArCrCrGrArUrArGrArArArUrArU | |
| A037 | LL10 | | rCrGrGrUrCrCrGrGrUrCrGrArGrGrArGrArGrCrArUrCrGr | |
| | | | GrUrCrUrGrArArGrGrArUrGrCrArCrUrCrCrGrGrGrArUrA | |
| | | | rGrGrGrCrArGrUrCrCrCrGrGrCrUrCrUrUrGrCrUrArArGr | |
| | | | UrCrCrUrUrCrArGrCrArArGrUrCrGrArArArGrArCrArCrG | |
| | | | rArUrGrUrGrArGrCrCrUrArUrUrUrU | |
| IID25_S | IID25_P | V151 | mG*mA*mC*rUrUrGrGrGrUrGrArGrCrGrArUrGrGrArCr | 721 |
| yn_gRN | T_5_TT | | GrUrCrUrCrArArUrCrArArCrCrGrArUrArGrArArArUrArU | |
| A038 | LL10 | | rCrGrGrUrCrCrGrGrUrUrGrArGrArArGrArGrCrArUrCrGr | |
| | | | GrCrUrGrArArGrGrArUrGrCrArCrUrCrCrGrGrGrArUrArG | |
| | | | rGrGrCrArGrUrCrCrCrGrGrCrUrCrUrUrGrCrUrArArGrUr | |
| | | | CrCrUrUrCrArGrCrArArGrCrGrArArArGrCrArCrGrArUrG | |
| | | | rUrGrArGrCrCrUrArUrUrUrU | |
| IID25_S | IID25_P | V152 | mG*mA*mC*rUrUrGrGrGrUrGrArGrCrGrArUrGrGrArCr | 722 |
| yn_gRN | T_5_TT | | GrUrCrUrCrArArUrCrArArCrCrGrArGrArArArUrCrGrGrU | |
| A039 | LL10 | | rCrCrGrGrUrUrGrArArArArGrArGrCrArUrCrGrGrCrUrGr | |
| | | | ArArGrGrArUrGrCrArCrUrCrCrGrGrGrArUrArGrGrGrCrA | |
| | | | rGrUrCrCrCrGrGrCrUrCrUrUrGrCrUrArArGrUrCrCrUrUr | |
| | | | CrArGrCrArArGrCrGrArArArGrCrArCrGrArUrGrUrGrArG | |
| | | | rCrCrUrArUrUrUrU | |
| IID25_S | IID25_P | V149 | mG*mG*mA*rCrCrUrCrCrArGrGrUrGrCrArGrArArGrUrG | 723 |
| yn_gRN | T_10_C1 | | rCrCrUrCrArArUrCrArArCrCrGrArUrArGrArArArUrArUr | |
| A040 | QTNF12 | | CrGrGrUrCrCrGrGrUrUrGrArGrGrArGrArGrCrArUrCrGrG | |
| | | | rUrCrUrGrArArGrGrArUrGrCrArCrUrCrCrGrGrGrArUrAr | |
| | | | GrGrGrCrArGrUrCrCrCrGrGrCrUrCrUrUrGrCrUrArArGrU | |
| | | | rCrCrUrUrCrArGrCrArArGrUrCrGrArArArGrArCrArCrGr | |
| | | | ArUrGrUrGrArGrCrCrUrArUrUrUrU | |
| IID25_S | IID25_P | V150 | mG*mG*mA*rCrCrUrCrCrArGrGrUrGrCrArGrArArGrUrG | 724 |
| yn_gRN | T_10_C1 | | rCrCrUrCrGrArUrCrArArCrCrGrArUrArGrArArArUrArUr | |
| A041 | QTNF12 | | CrGrGrUrCrCrGrGrUrCrGrArGrGrArGrArGrCrArUrCrGrG | |
| | | | rUrCrUrGrArArGrGrArUrGrCrArCrUrCrCrGrGrGrArUrAr | |
| | | | GrGrGrCrArGrUrCrCrCrGrGrCrUrCrUrUrGrCrUrArArGrU | |
| | | | rCrCrUrUrCrArGrCrArArGrUrCrGrArArArGrArCrArCrGr | |
| | | | ArUrGrUrGrArGrCrCrUrArUrUrUrU | |
| IID25_S | IID25_P | V151 | mG*mG*mA*rCrCrUrCrCrArGrGrUrGrCrArGrArArGrUrG | 725 |
| yn_gRN | T_10_C1 | | rUrCrUrCrArArUrCrArArCrCrGrArUrArGrArArArUrArUr | |
| A042 | QTNF12 | | CrGrGrUrCrCrGrGrUrUrGrArGrArArGrArGrCrArUrCrGrG | |
| | | | rCrUrGrArArGrGrArUrGrCrArCrUrCrCrGrGrGrArUrArGr | |
| | | | GrGrCrArGrUrCrCrCrGrGrCrUrCrUrUrGrCrUrArArGrUrC | |
| | | | rCrUrUrCrArGrCrArArGrCrGrArArArGrCrArCrGrArUrGr | |
| | | | UrGrArGrCrCrUrArUrUrUrU | |
| IID25_S | IID25_P | V152 | mG*mG*mA*rCrCrUrCrCrArGrGrUrGrCrArGrArArGrUrG | 726 |
| yn_gRN | T_10_C1 | | rUrCrUrCrArArUrCrArArCrCrGrArGrArArArUrCrGrGrUr | |
| A043 | QTNF12 | | CrCrGrGrUrUrGrArArArArGrArGrCrArUrCrGrGrCrUrGrA | |
| | | | rArGrGrArUrGrCrArCrUrCrCrGrGrGrArUrArGrGrGrCrAr | |
| | | | GrUrCrCrCrGrGrCrUrCrUrUrGrCrUrArArGrUrCrCrUrUrC | |
| | | | rArGrCrArArGrCrGrArArArGrCrArCrGrArUrGrUrGrArGr | |
| | | | CrCrUrArUrUrUrU | |
| IID25_S | IID25_P | V149 | mC*mU*mG*rUrGrArArGrArGrCrCrArGrUrGrGrCrCrArG | 727 |
| yn_gRN | T_15_CP | | rCrCrUrCrArArUrCrArArCrCrGrArUrArGrArArArUrArUr | |
| A044 | TP | | CrGrGrUrCrCrGrGrUrUrGrArGrGrArGrArGrCrArUrCrGrG | |
| | | | rUrCrUrGrArArGrGrArUrGrCrArCrUrCrCrGrGrGrArUrAr | |
| | | | GrGrGrCrArGrUrCrCrCrGrGrCrUrCrUrUrGrCrUrArArGrU | |
| | | | rCrCrUrUrCrArGrCrArArGrUrCrGrArArArGrArCrArCrGr | |
| | | | ArUrGrUrGrArGrCrCrUrArUrUrUrU | |
| IID25_S | IID25_P | V150 | mC*mU*mG*rUrGrArArGrArGrCrCrArGrUrGrGrCrCrArG | 728 |
| yn_gRN | T_15_CP | | rCrCrUrCrGrArUrCrArArCrCrGrArUrArGrArArArUrArUr | |
| A045 | TP | | CrGrGrUrCrCrGrGrUrCrGrArGrGrArGrArGrCrArUrCrGrG | |
| | | | rUrCrUrGrArArGrGrArUrGrCrArCrUrCrCrGrGrGrArUrAr | |
| | | | GrGrGrCrArGrUrCrCrCrGrGrCrUrCrUrUrGrCrUrArArGrU | |
| | | | rCrCrUrUrCrArGrCrArArGrUrCrGrArArArGrArCrArCrGr | |
| | | | ArUrGrUrGrArGrCrCrUrArUrUrUrU | |
| IID25_S | IID25_P | V151 | mC*mU*mG*rUrGrArArGrArGrCrCrArGrUrGrGrCrCrArG | 729 |
| yn_gRN | T_15_CP | | rUrCrUrCrArArUrCrArArCrCrGrArUrArGrArArArUrArUr | |
| A046 | TP | | CrGrGrUrCrCrGrGrUrUrGrArGrArArGrArGrCrArUrCrGrG | |
| | | | rCrUrGrArArGrGrArUrGrCrArCrUrCrCrGrGrGrArUrArGr | |
| | | | GrGrCrArGrUrCrCrCrGrGrCrUrCrUrUrGrCrUrArArGrUrC | |
| | | | rCrUrUrCrArGrCrArArGrCrGrArArArGrCrArCrGrArUrGr | |
| | | | UrGrArGrCrCrUrArUrUrUrU | |
| IID25_S | IID25_P | V152 | mC*mU*mG*rUrGrArArGrArGrCrCrArGrUrGrGrCrCrArG | 730 |
| yn_gRN | T_15_CP | | rUrCrUrCrArArUrCrArArCrCrGrArGrArArArUrCrGrGrUr | |
| A047 | TP | | CrCrGrGrUrUrGrArArArArGrArGrCrArUrCrGrGrCrUrGrA | |
| | | | rArGrGrArUrGrCrArCrUrCrCrGrGrGrArUrArGrGrGrCrAr | |
| | | | GrUrCrCrCrGrGrCrUrCrUrUrGrCrUrArArGrUrCrCrUrUrC | |
| | | | rArGrCrArArGrCrGrArArArGrCrArCrGrArUrGrUrGrArGr | |
| | | | CrCrUrArUrUrUrU | |
| IID25_S | TTR1 | v70 | mA*mG*mC*rCrArCrGrUrCrUrArCrArGrCrArGrGrGrCrG | 731 |
| yn_gRN | | | rUrUrUrCrArArUrCrArArCrCrGrArUrArGrArArArUrArUr | |
| A048 | | | CrGrGrUrCrCrGrGrUrUrGrArArArArGrArGrCrArUrCrGrG | |
| | | | rUrCrUrGrArArGrGrArUrGrCrArCrUrCrCrGrGrGrArUrAr | |
| | | | GrGrGrCrArGrUrCrCrCrGrGrCrUrCrUrUrGrCrUrArArGrU | |
| | | | rCrCrUrUrCrArGrCrArArGrUrCrGrArArArGrArCrArCrGr | |
| | | | ArUrGrUrGrArGrCrCrUrArUrUrUrU | |
| IID25_S | TTR6 | v70 | mA*mC*mA*rGrCrArGrGrGrCrUrGrCrCrUrCrGrGrArCrG | 732 |
| yn_gRN | | | rUrUrUrCrArArUrCrArArCrCrGrArUrArGrArArArUrArUr | |
| A049 | | | CrGrGrUrCrCrGrGrUrUrGrArArArArGrArGrCrArUrCrGrG | |
| | | | rUrCrUrGrArArGrGrArUrGrCrArCrUrCrCrGrGrGrArUrAr | |
| | | | GrGrGrCrArGrUrCrCrCrGrGrCrUrCrUrUrGrCrUrArArGrU | |
| | | | rCrCrUrUrCrArGrCrArArGrUrCrGrArArArGrArCrArCrGr | |
| | | | ArUrGrUrGrArGrCrCrUrArUrUrUrU | |
| IID25_S | TTR7 | v70 | mG*mA*mG*rGrGrArUrCrCrUrGrGrGrArGrCrCrCrUrUrG | 733 |
| yn_gRN | | | rUrUrUrCrArArUrCrArArCrCrGrArUrArGrArArArUrArUr | |
| A050 | | | CrGrGrUrCrCrGrGrUrUrGrArArArArGrArGrCrArUrCrGrG | |
| | | | rUrCrUrGrArArGrGrArUrGrCrArCrUrCrCrGrGrGrArUrAr | |
| | | | GrGrGrCrArGrUrCrCrCrGrGrCrUrCrUrUrGrCrUrArArGrU | |
| | | | rCrCrUrUrCrArGrCrArArGrUrCrGrArArArGrArCrArCrGr | |
| | | | ArUrGrUrGrArGrCrCrUrArUrUrUrU | |
| IID25 S | TTR1 | V118 | mA*mG*mC*rCrArCrGrUrCrUrArCrArGrCrArGrGrGrCrG | 734 |
| yn_gRN | | | rUrCrUrCrArArUrCrArArCrCrGrArUrArGrArArArUrArUr | |
| A051 | | | CrGrGrUrCrCrGrGrUrUrGrArGrArArGrArGrCrArUrCrGrG | |
| | | | rUrCrUrGrArArGrGrArUrGrCrArCrUrCrCrGrGrGrArUrAr | |
| | | | GrGrGrCrArGrUrCrCrCrGrGrCrUrCrUrUrGrCrUrArArGrU | |
| | | | rCrCrUrUrCrArGrCrArArGrUrCrGrArArArGrArCrArCrGr | |
| | | | ArUrGrUrGrArGrCrCrUrArUrUrUrU | |
| IID25 S | TTR6 | V118 | mA*mC*mA*rGrCrArGrGrGrCrUrGrCrCrUrCrGrGrArCrG | 735 |
| yn_gRN | | | rUrCrUrCrArArUrCrArArCrCrGrArUrArGrArArArUrArUr | |
| A052 | | | CrGrGrUrCrCrGrGrUrUrGrArGrArArGrArGrCrArUrCrGrG | |
| | | | rUrCrUrGrArArGrGrArUrGrCrArCrUrCrCrGrGrGrArUrAr | |
| | | | GrGrGrCrArGrUrCrCrCrGrGrCrUrCrUrUrGrCrUrArArGrU | |
| | | | rCrCrUrUrCrArGrCrArArGrUrCrGrArArArGrArCrArCrGr | |
| | | | ArUrGrUrGrArGrCrCrUrArUrUrUrU | |
| IID25 S | TTR7 | V118 | mG*mA*mG*rGrGrArUrCrCrUrGrGrGrArGrCrCrCrUrUrG | 736 |
| yn_gRN | | | rUrCrUrCrArArUrCrArArCrCrGrArUrArGrArArArUrArUr | |
| A053 | | | CrGrGrUrCrCrGrGrUrUrGrArGrArArGrArGrCrArUrCrGrG | |
| | | | rUrCrUrGrArArGrGrArUrGrCrArCrUrCrCrGrGrGrArUrAr | |
| | | | GrGrGrCrArGrUrCrCrCrGrGrCrUrCrUrUrGrCrUrArArGrU | |
| | | | rCrCrUrUrCrArGrCrArArGrUrCrGrArArArGrArCrArCrGr | |
| | | | ArUrGrUrGrArGrCrCrUrArUrUrUrU | |
| IID25 S | TTR1 +4 | v70 | mU*mU*mA*rCrArGrCrCrArCrGrUrCrUrArCrArGrCrArG | 737 |
| yn_gRN | | | rGrGrCrGrUrUrUrCrArArUrCrArArCrCrGrArUrArGrArAr | |
| A054 | | | ArUrArUrCrGrGrUrCrCrGrGrUrUrGrArArArArGrArGrCrA | |
| | | | rUrCrGrGrUrCrUrGrArArGrGrArUrGrCrArCrUrCrCrGrGr | |
| | | | GrArUrArGrGrGrCrArGrUrCrCrCrGrGrCrUrCrUrUrGrCrU | |
| | | | rArArGrUrCrCrUrUrCrArGrCrArArGrUrCrGrArArArGrAr | |
| | | | CrArCrGrArUrGrUrGrArGrCrCrUrArUrUrUrU | |
| IID25_S | TTR6 + 4 | v70 | mG*mU*mC*rUrArCrArGrCrArGrGrGrCrUrGrCrCrUrCrG | 738 |
| yn_gRN | | | rGrArCrGrUrUrUrCrArArUrCrArArCrCrGrArUrArGrArAr | |
| A055 | | | ArUrArUrCrGrGrUrCrCrGrGrUrUrGrArArArArGrArGrCrA | |
| | | | rUrCrGrGrUrCrUrGrArArGrGrArUrGrCrArCrUrCrCrGrGr | |
| | | | GrArUrArGrGrGrCrArGrUrCrCrCrGrGrCrUrCrUrUrGrCrU | |
| | | | rArArGrUrCrCrUrUrCrArGrCrArArGrUrCrGrArArArGrAr | |
| | | | CrArCrGrArUrGrUrGrArGrCrCrUrArUrUrUrU | |
| IID25_S | TTR7 + 4 | v70 | mC*mU*mC*rUrGrArGrGrGrArUrCrCrUrGrGrGrArGrCrC | 739 |
| yn_gRN | | | rCrUrUrGrUrUrUrCrArArUrCrArArCrCrGrArUrArGrArAr | |
| A056 | | | ArUrArUrCrGrGrUrCrCrGrGrUrUrGrArArArArGrArGrCrA | |
| | | | rUrCrGrGrUrCrUrGrArArGrGrArUrGrCrArCrUrCrCrGrGr | |
| | | | GrArUrArGrGrGrCrArGrUrCrCrCrGrGrCrUrCrUrUrGrCrU | |
| | | | rArArGrUrCrCrUrUrCrArGrCrArArGrUrCrGrArArArGrAr | |
| | | | CrArCrGrArUrGrUrGrArGrCrCrUrArUrUrUrU | |
| IID25_S | TTR1 + 4 | V118 | mU*mU*mA*rCrArGrCrCrArCrGrUrCrUrArCrArGrCrArG | 740 |
| yn_gRN | | | rGrGrCrGrUrCrUrCrArArUrCrArArCrCrGrArUrArGrArAr | |
| A057 | | | ArUrArUrCrGrGrUrCrCrGrGrUrUrGrArGrArArGrArGrCrA | |
| | | | rUrCrGrGrUrCrUrGrArArGrGrArUrGrCrArCrUrCrCrGrGr | |
| | | | GrArUrArGrGrGrCrArGrUrCrCrCrGrGrCrUrCrUrUrGrCrU | |
| | | | rArArGrUrCrCrUrUrCrArGrCrArArGrUrCrGrArArArGrAr | |
| | | | CrArCrGrArUrGrUrGrArGrCrCrUrArUrUrUrU | |
| IID25_S | TTR6 + 4 | V118 | mG*mU*mC*rUrArCrArGrCrArGrGrGrCrUrGrCrCrUrCrG | 741 |
| yn_gRN | | | rGrArCrGrUrCrUrCrArArUrCrArArCrCrGrArUrArGrArAr | |
| A058 | | | ArUrArUrCrGrGrUrCrCrGrGrUrUrGrArGrArArGrArGrCrA | |
| | | | rUrCrGrGrUrCrUrGrArArGrGrArUrGrCrArCrUrCrCrGrGr | |
| | | | GrArUrArGrGrGrCrArGrUrCrCrCrGrGrCrUrCrUrUrGrCrU | |
| | | | rArArGrUrCrCrUrUrCrArGrCrArArGrUrCrGrArArArGrAr | |
| | | | CrArCrGrArUrGrUrGrArGrCrCrUrArUrUrUrU | |
| IID25 S | TTR7+4 | V118 | mC*mU*mC*rUrGrArGrGrGrArUrCrCrUrGrGrGrArGrCrC | 742 |
| yn_gRN | | | rCrUrUrGrUrCrUrCrArArUrCrArArCrCrGrArUrArGrArAr | |
| A059 | | | ArUrArUrCrGrGrUrCrCrGrGrUrUrGrArGrArArGrArGrCrA | |
| | | | rUrCrGrGrUrCrUrGrArArGrGrArUrGrCrArCrUrCrCrGrGr | |
| | | | GrArUrArGrGrGrCrArGrUrCrCrCrGrGrCrUrCrUrUrGrCrU | |
| | | | rArArGrUrCrCrUrUrCrArGrCrArArGrUrCrGrArArArGrAr | |
| | | | CrArCrGrArUrGrUrGrArGrCrCrUrArUrUrUrU | |
| IID25_S | Tet | v70 | mC*mU*mC*rCrCrUrArUrCrArGrUrGrArUrArGrArGrArG | 743 |
| yn_gRN | reporter | | rUrUrUrCrArArUrCrArArCrCrGrArUrArGrArArArUrArUr | |
| A060 | | | CrGrGrUrCrCrGrGrUrUrGrArArArArGrArGrCrArUrCrGrG | |
| | | | rUrCrUrGrArArGrGrArUrGrCrArCrUrCrCrGrGrGrArUrAr | |
| | | | GrGrGrCrArGrUrCrCrCrGrGrCrUrCrUrUrGrCrUrArArGrU | |
| | | | rCrCrUrUrCrArGrCrArArGrUrCrGrArArArGrArCrArCrGr | |
| | | | ArUrGrUrGrArGrCrCrUrArUrUrUrU | |
| IID25_S | Tet | V118 | mC*mU*mC*rCrCrUrArUrCrArGrUrGrArUrArGrArGrArG | 744 |
| yn_gRN | reporter | | rUrCrUrCrArArUrCrArArCrCrGrArUrArGrArArArUrArUr | |
| A061 | | | CrGrGrUrCrCrGrGrUrUrGrArGrArArGrArGrCrArUrCrGrG | |
| | | | rUrCrUrGrArArGrGrArUrGrCrArCrUrCrCrGrGrGrArUrAr | |
| | | | GrGrGrCrArGrUrCrCrCrGrGrCrUrCrUrUrGrCrUrArArGrU | |
| | | | rCrCrUrUrCrArGrCrArArGrUrCrGrArArArGrArCrArCrGr | |
| | | | ArUrGrUrGrArGrCrCrUrArUrUrUrU | |
| IID25 S | TTR1 | V151 | mA*mG*mC*rCrArCrGrUrCrUrArCrArGrCrArGrGrGrCrG | 745 |
| yn_gRN | | | rUrCrUrCrArArUrCrArArCrCrGrArUrArGrArArArUrArUr | |
| A062 | | | CrGrGrUrCrCrGrGrUrUrGrArGrArArGrArGrCrArUrCrGrG | |
| | | | rCrUrGrArArGrGrArUrGrCrArCrUrCrCrGrGrGrArUrArGr | |
| | | | GrGrCrArGrUrCrCrCrGrGrCrUrCrUrUrGrCrUrArArGrUrC | |
| | | | rCrUrUrCrArGrCrArArGrCrGrArArArGrCrArCrGrArUrGr | |
| | | | UrGrArGrCrCrUrArUrUrUrU | |
| IID25 S | TTR6 | V151 | mA*mC*mA*rGrCrArGrGrGrCrUrGrCrCrUrCrGrGrArCrG | 746 |
| yn_gRN | | | rUrCrUrCrArArUrCrArArCrCrGrArUrArGrArArArUrArUr | |
| A063 | | | CrGrGrUrCrCrGrGrUrUrGrArGrArArGrArGrCrArUrCrGrG | |
| | | | rCrUrGrArArGrGrArUrGrCrArCrUrCrCrGrGrGrArUrArGr | |
| | | | GrGrCrArGrUrCrCrCrGrGrCrUrCrUrUrGrCrUrArArGrUrC | |
| | | | rCrUrUrCrArGrCrArArGrCrGrArArArGrCrArCrGrArUrGr | |
| | | | UrGrArGrCrCrUrArUrUrUrU | |
| IID25_S | TTR7 | V151 | mG*mA*mG*rGrGrArUrCrCrUrGrGrGrArGrCrCrCrUrUrG | 747 |
| yn_gRN | | | rUrCrUrCrArArUrCrArArCrCrGrArUrArGrArArArUrArUr | |
| A064 | | | CrGrGrUrCrCrGrGrUrUrGrArGrArArGrArGrCrArUrCrGrG | |
| | | | rCrUrGrArArGrGrArUrGrCrArCrUrCrCrGrGrGrArUrArGr | |
| | | | GrGrCrArGrUrCrCrCrGrGrCrUrCrUrUrGrCrUrArArGrUrC | |
| | | | rCrUrUrCrArGrCrArArGrCrGrArArArGrCrArCrGrArUrGr | |
| | | | UrGrArGrCrCrUrArUrUrUrU | |
| IID25 S | TTR1 | V151 | mA*mG*mC*rCrArCrGrUrCrUrArCrArGrCrArGrGrGrCrG | 748 |
| yn_gRN | | | rUrCrUrCrArArUrCrArArCrCrGrArUrArGrArArArUrArUr | |
| A065 | | | CrGrGrUrCrCrGrGrUrUrGrArGrArArGrArGrCrArUrCrGrG | |
| | | | rCrUrGrArArGrGrArUrGrCrArCrUrCrCrGrGrGrArUrArGr | |
| | | | GrGrCrArGrUrCrCrCrGrGrCrUrCrUrUrGrCrUrArArGrUrC | |
| | | | rCrUrUrCrArGrCrArArGrCrGrArArArGrCrArCrGrArUrGr | |
| | | | UrGrArGrCrCrUrArU*mU*mU*mU | |
| IID25 S | TTR6 | V151 | mA*mC*mA*rGrCrArGrGrGrCrUrGrCrCrUrCrGrGrArCrG | 749 |
| yn_gRN | | | rUrCrUrCrArArUrCrArArCrCrGrArUrArGrArArArUrArUr | |
| A066 | | | CrGrGrUrCrCrGrGrUrUrGrArGrArArGrArGrCrArUrCrGrG | |
| | | | rCrUrGrArArGrGrArUrGrCrArCrUrCrCrGrGrGrArUrArGr | |
| | | | GrGrCrArGrUrCrCrCrGrGrCrUrCrUrUrGrCrUrArArGrUrC | |
| | | | rCrUrUrCrArGrCrArArGrCrGrArArArGrCrArCrGrArUrGr | |
| | | | UrGrArGrCrCrUrArU*mU*mU*mU | |
| IID25 S | TTR7 | V151 | mG*mA*mG*rGrGrArUrCrCrUrGrGrGrArGrCrCrCrUrUrG | 750 |
| yn_gRN | | | rUrCrUrCrArArUrCrArArCrCrGrArUrArGrArArArUrArUr | |
| A067 | | | CrGrGrUrCrCrGrGrUrUrGrArGrArArGrArGrCrArUrCrGrG | |
| | | | rCrUrGrArArGrGrArUrGrCrArCrUrCrCrGrGrGrArUrArGr | |
| | | | GrGrCrArGrUrCrCrCrGrGrCrUrCrUrUrGrCrUrArArGrUrC | |
| | | | rCrUrUrCrArGrCrArArGrCrGrArArArGrCrArCrGrArUrGr | |
| | | | UrGrArGrCrCrU*mU*mU*mU | |
| IID25 S | TTR1 | V118 | mA*mG*mC*rCrArCrGrUrCrUrArCrArGrCrArGrGrGrCrG | 751 |
| yn_gRN | | | rUrCrUrCrArArUrCrArArCrCrGrArUrArGrArArArUrArUr | |
| A068 | | | CrGrGrUrCrCrGrGrUrUrGrArGrArArGrArGrCrArUrCrGrG | |
| | | | rUrCrUrGrArArGrGrArUrGrCrArCrUrCrCrGrGrGrArUrAr | |
| | | | GrGrGrCrArGrUrCrCrCrGrGrCrUrCrUrUrGrCrUrArArGrU | |
| | | | rCrCrUrUrCrArGrCrArArGrUrCrGrArArArGrArCrArCrGr | |
| | | | ArUrGrUrGrArGrCrCrUrArU*mU*mU*mU | |
| IID25_S | TTR6 | V118 | mA*mC*mA*rGrCrArGrGrGrCrUrGrCrCrUrCrGrGrArCrG | 752 |
| yn_gRN | | | rUrCrUrCrArArUrCrArArCrCrGrArUrArGrArArArUrArUr | |
| A069 | | | CrGrGrUrCrCrGrGrUrUrGrArGrArArGrArGrCrArUrCrGrG | |
| | | | rUrCrUrGrArArGrGrArUrGrCrArCrUrCrCrGrGrGrArUrAr | |
| | | | GrGrGrCrArGrUrCrCrCrGrGrCrUrCrUrUrGrCrUrArArGrU | |
| | | | rCrCrUrUrCrArGrCrArArGrUrCrGrArArArGrArCrArCrGr | |
| | | | ArUrGrUrGrArGrCrCrUrArU*mU*mU*mU | |
| IID25_S | TTR7 | V118 | mG*mA*mG*rGrGrArUrCrCrUrGrGrGrArGrCrCrCrUrUrG | 753 |
| yn_gRN | | | rUrCrUrCrArArUrCrArArCrCrGrArUrArGrArArArUrArUr | |
| A070 | | | CrGrGrUrCrCrGrGrUrUrGrArGrArArGrArGrCrArUrCrGrG | |
| | | | rUrCrUrGrArArGrGrArUrGrCrArCrUrCrCrGrGrGrArUrAr | |
| | | | GrGrGrCrArGrUrCrCrCrGrGrCrUrCrUrUrGrCrUrArArGrU | |
| | | | rCrCrUrUrCrArGrCrArArGrUrCrGrArArArGrArCrArCrGr | |
| | | | ArUrGrUrGrArGrCrCrUrArU*mU*mU*mU | |
| IID25_S | TTR1 + 4 | V118 | mU*mU*mA*rCrArGrCrCrArCrGrUrCrUrArCrArGrCrArG | 754 |
| yn_gRN | | | rGrGrCrGrUrCrUrCrArArUrCrArArCrCrGrArUrArGrArAr | |
| A071 | | | ArUrArUrCrGrGrUrCrCrGrGrUrUrGrArGrArArGrArGrCrA | |
| | | | rUrCrGrGrUrCrUrGrArArGrGrArUrGrCrArCrUrCrCrGrGr | |
| | | | GrArUrArGrGrGrCrArGrUrCrCrCrGrGrCrUrCrUrUrGrCrU | |
| | | | rArArGrUrCrCrUrUrCrArGrCrArArGrUrCrGrArArArGrAr | |
| | | | CrArCrGrArUrGrUrGrArGrCrCrUrArU*mU*mU*mU | |
| IID25_S | TTR6 + 4 | V118 | mG*mU*mC*rUrArCrArGrCrArGrGrGrCrUrGrCrCrUrCrG | 755 |
| yn_gRN | | | rGrArCrGrUrCrUrCrArArUrCrArArCrCrGrArUrArGrArAr | |
| A072 | | | ArUrArUrCrGrGrUrCrCrGrGrUrUrGrArGrArArGrArGrCrA | |
| | | | rUrCrGrGrUrCrUrGrArArGrGrArUrGrCrArCrUrCrCrGrGr | |
| | | | GrArUrArGrGrGrCrArGrUrCrCrCrGrGrCrUrCrUrUrGrCrU | |
| | | | rArArGrUrCrCrUrUrCrArGrCrArArGrUrCrGrArArArGrAr | |
| | | | CrArCrGrArUrGrUrGrArGrCrCrUrArU*mU*mU*mU | |
| IID25 S | TTR7 + 4 | V118 | mC*mU*mC*rUrGrArGrGrGrArUrCrCrUrGrGrGrArGrCrC | 756 |
| yn_gRN | | | rCrUrUrGrUrCrUrCrArArUrCrArArCrCrGrArUrArGrArAr | |
| A073 | | | ArUrArUrCrGrGrUrCrCrGrGrUrUrGrArGrArArGrArGrCrA | |
| | | | rUrCrGrGrUrCrUrGrArArGrGrArUrGrCrArCrUrCrCrGrGr | |
| | | | GrArUrArGrGrGrCrArGrUrCrCrCrGrGrCrUrCrUrUrGrCrU | |
| | | | rArArGrUrCrCrUrUrCrArGrCrArArGrUrCrGrArArArGrAr | |
| | | | CrArCrGrArUrGrUrGrArGrCrCrUrArU*mU*mU*mU | |

TABLE 8
Primers

| | | | SEQ ID |
|---|---|---|---|
| Primer | Description | Sequence | NO |
| HK_ | mTTR | ACACTCTTTCCCTACACG | 757 |
| Miseq043 | Exon2_F | ACGCTCTTCCGATCTTT | |
| | | TCA CTG ACA TTT | |
| | | CTC TTG TCT | |
| HK_ | mTTR | GTGACTGGAGTTCAGACG | 758 |
| Miseq044 | Exon2_R | TGTGCTCTTCCGATCTGT | |
| | | GTG TCT GAA TCT AAC | |
| | | ACG AAT | |

What is claimed is:

1. A Cas9-IID system comprising: a) a Cas9-IID proteins effector polypeptide, or one or more nucleotide sequences encoding the protein effector polypeptide, wherein the protein effector polypeptide comprises an amino acid sequence SEQ ID NO:9, or comprises a variant of a protein effector comprising an amino acid sequence having at least 60%, 65%, 70%, 75%, 80%, 85%, 90%, or 95% identity to an amino acid sequence selected from the group consisting of SEQ ID NO:9, or comprises the amino acid sequence of a naturally-occurring protein effector having at least 30% identity to an amino acid sequence selected from the group consisting of SEQ ID NO:9, and b) one or more engineered guide RNAs comprising a guide sequence, wherein the one or more guide RNAs is designed to form a complex with the protein effector polypeptide and wherein the one or more guide RNAs comprises a guide sequence (also referred to as guide ribonucleic acid sequence) designed to hybridize with one or more target nucleic acid molecules, wherein the guide RNA and the protein effector polypeptide do not naturally occur together.

2. The Cas9-IID system of claim 1, wherein the amino acid sequence of the Cas9-IID protein comprises at least one, at least two, at least three, at least four, at least five, at least six, at least seven, at least eight, or at least nine mutation(s) at position(s) 7, 55, 56, 57, 58, 59, 60, 61, 62, 63, 64, 65, 66, 624, 858, 859, 860, 861, 862, 863, 864, 865, 866, 867, 868, 896, 897, 898, 899, 900, 901, 902, 903, and/or 904 of the amino acid sequence of SEQ ID NO: 9.

3. An engineered, non-naturally occurring Cas9-IID system comprising one or more nucleic acid constructs comprising:
a) a polynucleotide sequence encoding a protein effector polypeptide; wherein the protein effector polypeptide comprises an amino acid sequence selected from the group comprising SEQ ID NO:9, or comprises a variant of a protein effector comprising an amino acid sequence having at least 60%, 65%, 70%, 75%, 80%, 85%, 90%, or 95% identity to an amino acid sequence selected from the group consisting of SEQ ID NO:9, or comprises the amino acid sequence of a naturally-occurring protein effector having at least 50% identity to an amino acid sequence selected from the group consisting of SEQ ID NO:9, and b) a polynucleotide sequence encoding a guide RNA, wherein the guide RNA is designed to form a complex with the protein effector polypeptide and wherein the guide RNA comprises a guide sequence designed to hybridize with one or more target nucleic acid molecules, wherein the guide RNA and the protein effector polypeptide do not naturally occur together.

4. An engineered, non-naturally occurring Cas9-IID system according to claim 3, wherein the polynucleotide sequence encoding the protein polypeptide and the polynucleotide sequence encoding a guide RNA are located on the same or different nucleic acid constructs of the system, wherein when transcribed, the one or more guide RNAs forms one or more complexes with the Protein effector polypeptide, and wherein the one or more guide RNAs hybridizes to the one or more target nucleic acid molecules, resulting in cleavage of the target nucleic acid molecule.

5. An engineered, non-naturally occurring Cas9-IID system comprising one or more nucleic acid constructs comprising:
a) a polynucleotide sequence encoding a Cas9-IID protein effector polypeptide; wherein the protein effector polypeptide comprises an amino acid sequence selected from the group comprising SEQ ID NO: 1, SEQ ID NO:2, SEQ ID NO:3, SEQ ID NO:4, SEQ ID NO:5, SEQ ID NO:6, SEQ ID NO:7, SEQ ID NO:8, SEQ ID NO: 10, SEQ ID NO: 11 and SEQ ID NO:13, or comprises a variant of a Cas9-IID protein effector comprising an amino acid sequence having at least 60%, 65%, 70%, 75%, 80%, 85%, 90%, or 95% identity to an amino acid sequence selected from the group consisting of SEQ ID NO: 1, SEQ ID NO:2, SEQ ID NO:3, SEQ ID NO:4, SEQ ID NO:5, SEQ ID NO:6, SEQ ID NO:7, SEQ ID NO:8, SEQ ID NO:10, SEQ ID NO: 11 and SEQ ID NO:13; or comprises the amino acid sequence of a naturally-occurring Cas9-IID protein effector having at least 50% identity to an amino acid sequence selected from the group consisting of SEQ ID NO: 1, SEQ ID NO:2, SEQ ID NO:3, SEQ ID NO:4, SEQ ID NO:5, SEQ ID NO:6, SEQ ID NO:7, SEQ ID NO:8, SEQ ID NO:10, SEQ ID NO: 11 and SEQ ID NO:13, and b) a polynucleotide sequence encoding a guide RNA, wherein the guide RNA is designed to form a complex with the protein effector polypeptide and wherein the guide RNA comprises a guide sequence designed to hybridize with one or more target nucleic acid molecules, wherein the guide RNA and the protein effector polypeptide do not naturally occur together, and wherein and wherein the amino acid sequence of the Cas9-IID protein comprises at least one, at least two, at least three, at least four, at least five, at least six, at least seven, at least eight, or at least nine mutation(s) at position(s) 7, 45, 46, 47, 48, 49, 50, 51, 52, 53, 54, 55, 56, 558, 842, 843, 844, 845, 846, 847, 848, 849, 850, 851, 852, 853, 854, 908, 910, 911, 912, 913, 914, 915, 916, 917, 918, 919, 920, and/or 921.

6. An engineered, non-naturally occurring Cas9-IID system according to claim 5, wherein the polynucleotide sequence encoding the protein polypeptide and the polynucleotide sequence encoding a guide RNA are located on the same or different nucleic acid constructs of the system, wherein when transcribed, the one or more guide RNAs forms one or more complexes with the Protein effector polypeptide, and wherein the one or more guide RNAs hybridizes to the one or more target nucleic acid molecules, resulting in cleavage of the target nucleic acid molecule.

7. An engineered, non-naturally occurring Cas9-IID system comprising one or more nucleic acid constructs comprising:
a) a polynucleotide sequence encoding a Cas9-IID protein effector polypeptide; wherein the protein effector polypeptide comprises an amino acid sequence selected from the group comprising SEQ ID NO: 12, or comprises a variant of a Cas9-IID protein effector comprising an amino acid sequence having at least 60%, 65%, 70%, 75%, 80%, 85%, 90%, or 95% identity to an amino acid sequence selected from the group consisting of SEQ ID NO: 12, or comprises the amino acid sequence of a naturally-occurring Cas9-IID protein effector having at least 50% identity to an amino acid sequence selected from the group consisting of SEQ ID NO: 12, and b) a polynucleotide sequence encoding a guide RNA, wherein the guide RNA is designed to form a complex with the protein effector polypeptide and wherein the guide RNA comprises a guide sequence designed to hybridize with one or more target nucleic acid molecules, wherein the guide RNA and the protein effector polypeptide do not naturally occur together, and wherein and wherein the amino acid sequence of the Cas9-IID protein comprises at least one, at least two, at least three, at least four, at least five, at least six, at least seven, at least eight, or at least nine mutation(s) at position(s) 7, 45, 46, 47, 48, 49, 50, 51, 52, 53, 54, 55, 56, 558, 843, 844, 845, 846, 847, 848, 849, 850, 851, 852, 853, 854, 855, 909, 910, 911, 912, 913, 914, 915, 916, 917, 918, 919, 920, 921, and/or 922 of the amino acid sequence of SEQ ID NO: 12.

8. An engineered, non-naturally occurring Cas9-IID system according to claim 5, wherein the polynucleotide sequence encoding the protein polypeptide and the polynucleotide sequence encoding a guide RNA are located on the same or different nucleic acid constructs of the system, wherein when transcribed, the one or more guide RNAs forms one or more complexes with the Protein effector polypeptide, and wherein the one or more guide RNAs hybridizes to the one or more target nucleic acid molecules, resulting in cleavage of the target nucleic acid molecule.

9. An engineered, non-naturally occurring Cas9-IID system comprising one or more nucleic acid constructs comprising:
a) a polynucleotide sequence encoding a Cas9-IID protein effector polypeptide; wherein the protein effector polypeptide comprises an amino acid sequence selected from the group comprising SEQ ID NO: 1, or comprises a variant of a Cas9-IID protein effector comprising an amino acid sequence having at least 60%, 65%, 70%, 75%, 80%, 85%, 90%, or 95% identity to an amino acid sequence selected from the group consisting of SEQ ID NO: 1, or comprises the amino acid sequence of a naturally-occurring Cas9-IID protein effector having at least 50% identity to an amino acid sequence selected from the group consisting of SEQ ID NO: 1, and b) a polynucleotide sequence encoding a guide RNA, wherein the guide RNA is designed to form a complex with the protein effector polypeptide and wherein the guide RNA comprises a guide sequence designed to hybridize with one or more target nucleic acid molecules, wherein the guide RNA and the protein effector polypeptide do not naturally occur together, and wherein and wherein the amino acid sequence of the Cas9-IID protein comprises at least one, at least two, at least three, at least four, at least five, at least six, at least seven, at least eight, or at least nine mutation(s) at position(s) 13, 19, 20, 40, 69, 70, 71, 89, 105, 106, 131, 153, 180, 185, 215, 221, 239, 302, 366, 367, 370, 372, 376, 387 410, 458, 469, 473, 495, 537, 538, 571, 598, 609, 610, 611, 657, 700, 736, 737, 786, 800, 821, 827, 828, 843, 866, 873, 901, 913, 928, and 930 of the amino acid sequence of SEQ ID NO: 1.

10. The Cas9-IID system of any of claims 1-9, further comprising a target nucleic acid molecule.

11. The Cas9-IID system of claim 10, wherein the target nucleic acid molecule is a prokaryotic target nucleic acid molecule.

12. The Cas9-IID system of claim 10, wherein the target nucleic acid molecule is a eukaryotic target nucleic acid molecule.

13. The Cas9-IID system of claim 10, wherein the target nucleic acid molecule is within a cell.

14. The Cas9-IID system of claim 13, wherein the cell is a prokaryotic cell.

15. The Cas9-IID system of claim 13, wherein the cell is a eukaryotic cell.

16. The Cas9-IID system of claim 15, wherein the nucleotide sequence encoding the Cas9-IID protein effector polypeptide is codon optimized for expression in a eukaryotic cell.

17. The Cas9-IID system of any one of claims 1-16, further comprising one or more guide RNAs.

18. The Cas9-IID system of any of claims 1-17, wherein the polynucleotide sequence encoding a guide RNA encodes one or more guide RNAs.

19. The Cas9-IID system of any one of claims 1-18, wherein said engineered guide ribonucleic acid structure (e.g., guide RNA) comprises a single ribonucleic acid polynucleotide comprising said guide ribonucleic acid sequence and a tracr sequence (e.g., tracr ribonucleic acid sequence).

20. The Cas9-IID system of any one of claims 1-19, wherein said guide ribonucleic acid sequence is complementary to a prokaryotic, bacterial, archaeal, eukaryotic, fungal, plant, mammalian, or human genomic sequence.

21. The Cas9-IID system of any one of claims 1-20, wherein said guide ribonucleic acid sequence is 15-25 nucleotides in length.

22. The Cas9-IID system of any one of claims 1-21, wherein said endonuclease comprises one or more nuclear localization sequences (NLSs) proximal to an N- or C-terminus of said Cas9-IID nuclease.

23. The Cas9-IID of any one of claims 1-22, further comprising a single- or double-stranded DNA repair template comprising from 5′ to 3′: a first homology arm comprising a sequence of at least 20 nucleotides 5′ to said target deoxyribonucleic acid sequence, a synthetic DNA sequence of at least 10 nucleotides, and a second homology arm comprising a sequence of at least 20 nucleotides 3′ to said target sequence.

24. The Cas9-IID system of claim 23, wherein said first or second homology arm comprises a sequence of at least 40, 80, 120, 150, 200, 300, 500, or 1,000 nucleotides.

25. The Cas9-IID system of any one of claims 1-24, wherein said system further comprises a source of Mg2+.

26. The Cas9-IID system of any one of claims 1-25 wherein said Cas9-IID and said tracr ribonucleic acid sequence are derived from distinct bacterial species within a same phylum.

27. The Cas9-IID system of claim 26, wherein said guide RNA structure further comprises a second stem and a second loop, wherein the second stem comprises at least 5 pairs of ribonucleotides.

28. The Cas9-IID system of claim 27, wherein said guide RNA structure further comprises an RNA structure comprising at least two hairpins.

29. A deoxyribonucleic acid polynucleotide encoding the engineered guide ribonucleic acid polynucleotide of any one of claims 1-28 or 65.

30. A method for binding, cleaving, marking, or modifying a double-stranded deoxyribonucleic acid polynucleotide, comprising: (a) contacting said double-stranded deoxyribonucleic acid polynucleotide with a Cas9-IID endonuclease in complex with an engineered guide ribonucleic acid structure configured to bind to said Cas9-IID endonuclease and said double-stranded deoxyribonucleic acid polynucleotide; (b) wherein said double-stranded deoxyribonucleic acid polynucleotide comprises a protospacer adjacent motif (PAM); and wherein the Cas9-IID endonuclease is selected from the group comprising SEQ ID NO: 1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO: 12, and SEQ ID NO:13 with at least one mutation at position 7, 45, 46, 47, 48, 49, 50, 51, 52, 53, 54, 55, 56, 558, 842, 843, 844, 845, 846, 847, 848, 849, 850, 851, 852, 853, 854, 908, 910, 911, 912, 913, 914, 915, 916, 917, 918, 919, 920, and/or 921.

31. The method of claim 30, wherein said Cas9-IID endonuclease cleaves said double-stranded deoxyribonucleic acid polynucleotide, wherein said PAM comprises NGG, NACC, NVC, NRGM, NAC, NVCCC, NAV, NVC, or NAC.

32. The method of claim 30 or claim 31, wherein said Cas9-IID endonuclease cleaves said double-stranded deoxyribonucleic acid polynucleotide 6-8 nucleotides or 7 nucleotides from said PAM.

33. A method of modifying a target nucleic acid locus, said method comprising delivering to said target nucleic acid locus said engineered Cas9-IID system of any one of claims 1-29, wherein said Cas9-IID is configured to form a complex with said engineered guide ribonucleic acid structure, and wherein said complex is configured such that upon binding of said complex to said target nucleic acid locus, said complex modifies said target nucleic locus.

34. The method of claim 33, wherein modifying said target nucleic acid locus comprises binding, nicking, cleaving, or marking said target nucleic acid locus.

35. The method of claim 33 or claim 34, wherein said target nucleic acid locus comprises deoxyribonucleic acid (DNA) or ribonucleic acid (RNA).

36. The method of any one of claim 33-35, wherein delivering said engineered Cas9-IID system to said target nucleic acid locus comprises delivering a translated polypeptide.

37. A Cas9-IID system comprising: a) a Cas9-IID proteins effector polypeptide, or one or more nucleotide sequences encoding the protein effector polypeptide, wherein the protein effector polypeptide comprises an amino acid sequence SEQ ID NO:1, or comprises a variant of a protein effector comprising an amino acid sequence having at least 60%, 65%, 70%, 75%, 80%, 85%, 90%, or 95% identity to an amino acid sequence selected from the group consisting of SEQ ID NO:1, or comprises the amino acid sequence of a naturally-occurring protein effector having at least 70% identity to an amino acid sequence selected from the group consisting of SEQ ID NO:1, and b) one or more engineered guide RNA, wherein said engineered guide RNA comprises a structure A: 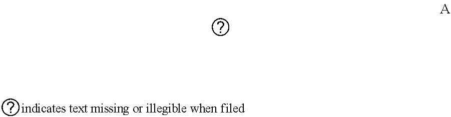
wherein N20 is a spacer sequence of 15-25 nucleotides at the 5′-end; wherein S1, S2, S3, S4, S5 and S6 comprise independently 2-20 nucleotide base-pairs; and wherein the engineered guide RNA is less than 155 nucleotides and comprising at least one modification.

38. The Cas9-IID system of claim 37, wherein said modification comprises deletion of one to four base-pairs in the S1.

39. The Cas9-IID system of claim 37, wherein said modification comprises deletion of S5 and/or S6.

40. The Cas9-IID system of claim 37, wherein said modification comprises deletion of at least three nucleotides at the 3′-end.

41. The Cas9-IID system of claim 37, wherein said modification comprises deletion of two nucleotide base-pairs in the S1, deletion of 5 nucleotides at the 3′-end, and deletion of the U in the S3 and deletion of S5 and S6.

42. The Cas9-IID system of claim 37, wherein the guide ribonucleic acid sequence is 20 or 21 nucleotides in length.

43. The Cas9-IID system of claim 37, wherein the deleted nucleotides are replaced with a linker (e.g., a loop portion or pin-loop).

44. The Cas9-IID system of claim 43, wherein the linker is single-stranded nucleic acid comprising from about from about 4 nucleotides to about 15 nucleotides.

45. The Cas9-IID system of claim 43, wherein a loop portion (pin-loop) of the 5′-stem-loop comprises from 4, 5 or 6 nucleotides.

46. The Cas9-IID system of claim 43, wherein a loop portion of the 5′-stem-loop comprises the nucleotide sequence GAAA or GAAAA.

47. The Cas9-IID system of claim 43, wherein a loop region (pin-loop) of the 5′-stem-loop comprises a nucleic acid modification.

48. The Cas9-IID system of claim 37, wherein said nucleic acid modification is a modified nucleotide selected from the group consisting of 2′-O-methyl (2′-OMe) nucleotides, 2′-fluoro (2′-F) nucleotides, bridged nucleic acid (BNA) nucleotides (e.g., 2′-O,4′-C-methylene (locked nucleic acid, LNA) nucleotides, 2′-O,4′-C-ethylene (locked nucleic acid, ENA) nucleotides, 5′-methyl-BNA, cEt BNA, cMOE BNA, oxy amino BNA and vinyl-carbo BNA), anhydrohexitol (1,5-anhydrohexitol nucleic acid, HNA) nucleotides, cyclohexene (Cyclohexene nucleic acid, CeNA) nucleotides, 2′-methoxyethyl (2′-MOE) nucleotides, 2′-O-allyl nucleotides, 2′-C-allyl ribose nucleotides, 2′-O—N-methylacetamido (2′-O-NMA) nucleotides, a 2′-O-dimethylaminoethoxyethyl (2′-O-DMAEOE) nucleotides, 2′-O-aininopropyl (2′-O-AP) nucleotides, 2′-F arabinose (2′-ara-F) nucleotides, threose (Threose nucleic acid, TNA) nucleotides, and acyclic nucleotides (e.g., unlocked nucleic acids (UNA) and 2,3-dihydroxylpropyl (glycol nucleic acid, GNA)); a modified internucleoside linkage; a non-natural or modified nucleobase; or a combination thereof.

49. The Cas9-IID system of claim 37, wherein said nucleic acid modification is 2′-O-methyl (2′-OMe).

50. The Cas9-IID system of claim 37, wherein said nucleic acid modification is 2′-fluoro modified nucleotide.

51. The Cas9-IID system of claim 37, wherein said nucleic acid modification is non-natural or modified nucleobase.

52. The Cas9-IID system of claim 37, wherein modification comprises at least one (e.g., 1, 2, 3, 4, 5, or more) modified internucleoside linkage (e.g., a modified internucleoside linkage selected independently from the group consisting of phosphorothioates (R, S, or racemic), phosphorodithioates, methylenemethylimino (MMI, 3′-CH₂—N(CH₃)—O-5′), phosphotriesters, alkylphosphonates (e.g., methylphosphonates), phosphoramidate, methylenemethylimino (—CH₂—N(CH₃)—O—CH₂—), thiodiester (—O—C(O)—S—), thionocarbamate (—O—C(O)(NH)—S—), siloxane (—O—Si(H)₂—O— and dialkylsiloxane), N,N′-dimethylhydrazine (—CH₂—N(CH₃)—N(CH₃)—), amide-3 (3′-CH₂—C(═O)—N(H)-5′), amide-4 (3′-CH₂—N(H)—C(═O)-5′)), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methykenecarbonylamino, methylenehydrazo, methylenedimethylhydrazo, methyleneoxymethylimino, ethers (C3′-O—C5′), thioethers (C3′-S—C5′), thioacetamido (C3′-N(H)—C(═O)—CH₂—S—C5′, C3′-O—P(O)—O—SS—C5′), C3′-CH₂—NH—NH—C5′, 3′-NHP(O)(OCH₃)—O-5′, 3′-NHP(O)(OCH₃)—O-5′), 2′->5′ internucleoside linkages, 2′->3′ internucleoside linkages, 3′->3′ internucleoside linkages, and 5′->5′ internucleoside linkages, optionally the modified internucleoside linkage is phosphorothioate, imidp or MMI, more preferably the modified internucleoside linkage is phosphorothioate (PS).

53. The Cas9-IID system of claim 37, wherein modification comprises a duplex stabilizing modification, optionally the duplex stabilizing modification is 2′-F nucleotide, 2′-OMe nucleotide, 2′-methoxyethyl nucleotide, 2,6-diaminopurine nucleotide, 5-methyl cytidine, N4-ethyl cytidine, 5-propynyl cytidine, 5-propynyl uridine, 5-hydroxybutynl-2′-deoxyuridine, 8-aza-7-deazaguanosine, a locked nucleic acid (LNA), and/or covalent cross-linking of two strands of the duplex.

54. The Cas9-IID system of claim 37, wherein the modification is at the 3′ end of the guide RNA and comprises any one of:
(i) a modification of any one or more of the last 7, 6, 5, 4, 3, 2, or 1 nucleotides;
(ii) one modified nucleotide;
(iii) two modified nucleotides;
(iv) three modified nucleotides;
(v) four modified nucleotides;
(vi) five modified nucleotides;
(vii) six modified nucleotides; or
(viii) seven modified nucleotides.

55. The Cas9-IID system of claim 37, wherein the modification is at the 3′ end of the guide RNA and comprises any one of:
(i) a modified internucleoside linkage (e.g., PS, imidp or MMI linkage) between nucleotides;
(ii) a 2′-OMe nucleotide;
(iii) a 2′-O-MOE nucleotide;
(iv) a 2′-F nucleotide;
(v) a LNA nucleotide
(vi) a 3′->3′ linkage between nucleotides;
(vii) an inverted abasic nucleotide; and
(viii) a combination of one or more of (i)-(vii).

56. The Cas9-IID system of claim 37, wherein the modification is at the 3′ end of the guide RNA and comprises any one of:
(i) a modified internucleoside linkage (e.g., PS, imidp or MMI linkage) between the last and second to last nucleotides;
(ii) a modified internucleoside linkage (e.g., PS, imidp or MMI linkage) between the last and second to last nucleotide and a PS linkage between the second and third to last nucleotides;
(iii) modified internucleoside linkages (e.g., PS and/or MMI linkage) between any one or more of the last four nucleotides;
(iv) modified internucleoside linkages (e.g., PS and/or MMI linkage) between any one or more of the last five nucleotides; or
(v) modified internucleoside linkages (e.g., PS and/or MMI linkage) between any one or more of the last 2, 3, 4, 5, 6, 7, 8, 9, or 10 nucleotides.

57. The Cas9-IID system of claim 37, wherein the modification is at the 3′ end of the guide RNA and comprises any one of:
(i) a modification of one or more of the last 1-7 nucleotides, wherein the modification is a modified internucleoside linkages (e.g., PS and/or MMI linkage), inverted abasic nucleotide, a 3′->3′ internucleoside linkage, 2′-OMe, 2′-O-MOE, 2′-F, LNA, or a combination thereof;
(ii) a modification to the last nucleotide with 2′-OMe, 2′-O-MOE, 2′-F, LNA, or combinations thereof, and an optional one or two modified internucleoside linkages (e.g., PS and/or MMI linkage) to the next nucleotide;
(iii) a modification to the last and/or second to last nucleotide with 2′-OMe, 2′-O-MOE, 2′-F, LNA, or combinations thereof, and optionally one or more modified internucleoside linkages (e.g., PS and/or MMI linkage); iv. a modification to the last, second to last, and/or third to last nucleotides with 2′-OMe, 2′-O-moe, 2′-F, or combinations thereof, and optionally one or more modified internucleoside linkages (e.g., PS and/or MMI linkage);
(v) a modification to the last, second to last, third to last, and/or fourth to last nucleotides with 2′-OMe, 2′-O-MOE, 2′-F, LNA or combinations thereof, and optionally one or more modified internucleoside linkages (e.g., PS and/or MMI linkage); or
(vi) a modification to the last, second to last, third to last, fourth to last, and/or fifth to last nucleotides with 2′-OMe, 2′-O-MOE, 2′-F, LNA, or combinations thereof, and optionally one or more modified internucleoside linkages (e.g., PS and/or MMI linkage).

58. The Cas9-IID system of claim 37, wherein the modification is at the 3′ end of the guide RNA and comprises any one of:
(i) a 2′-OMe modified nucleotide at the last position, three consecutive 2′-O-MOE modified nucleotides immediately 5′ to the 2′-OMe modified nucleotide, and three consecutive PS linkages between the last three nucleotides;
(ii) five consecutive 2′-OMe modified nucleotides from the 3′ end of the 3′ terminus, and three PS linkages between the last three nucleotides;
(iii) an inverted abasic modified nucleotide at the last position;
(iv) an inverted abasic modified nucleotide at the last position, and three consecutive 2′-OMe modified nucleotides at the last three positions
(v) 15 consecutive 2′-OMe modified nucleotides from the 3′ end, five consecutive 2′-F modified nucleotides immediately 5′ to the 2′-OMe modified nucleotides, and three PS linkages between the last three nucleotides;
(vi) alternating 2′-OMe modified nucleotides and 2′-F modified nucleotides at the last 20 nucleotides, and three PS linkages between the last three nucleotides;
(vii) two or three consecutive 2′-OMe modified nucleotides, and three PS linkages between the last three nucleotides;
(viii) one PS linkage between the last and next to last nucleotides; and
(ix) 15 or 20 consecutive 2′-OMe modified nucleotides, and three PS linkages between the last three nucleotides.

59. The Cas9-IID system of claim 37, wherein the modification is at the 5′ end of the guide RNA and comprises any one of:
(i) one modified nucleotide;
(ii) two modified nucleotides;
(iii) three modified nucleotides;
(iv) four modified nucleotides;
(v) five modified nucleotides;
(vi) six modified nucleotides; and
(vii) seven modified nucleotides.

60. The Cas9-IID system of claim 37, wherein the modification is at the 5′ end of the guide RNA and comprises any one or more modification of between 1 and 7, between 1 and 5, between 1 and 4, between 1 and 3, or between 1 and 2 nucleotides.

61. The Cas9-IID system of claim 37, wherein the modification is at the 5′ end of the guide RNA and comprises any one or more of:
(i) a modified internucleoside linkage (e.g., a phosphorothioate and/or MMI linkage) between nucleotides;
(ii) a 2′-OMe nucleotide;
(iii) a 2′-O-MOE nucleotide;
(iv) a 2′-F nucleotide;
(v) a LNA;
(vi) a 3′->3′ linkage;
(vii) an inverted abasic modified nucleotide;
(viii) a deoxyribonucleotide;
(ix) an inosine; and
(x) combinations of one or more of (i)-(ix).

62. The Cas9-IID system of claim 37, wherein the modification is at the 5′ end of the guide RNA and comprises:
(i) 1, 2, 3, 4, 5, 6, and/or 7 modified internucleoside (e.g., a phosphorothioate and/or MMI) linkages between nucleotides; or
(ii) about 1-2, 1-3, 1-4, 1-5, 1-6, or 1-7 modified internucleoside (e.g., a phosphorothioate and/or MMI) linkages between nucleotides.

63. The Cas9-IID system of claim 37, wherein the modification is at the 5′ end of the guide RNA and comprises:
(i) one modified internucleoside (e.g., a phosphorothioate and/or MMI) linkage, and the linkage is between nucleotides 1 and 2;
(ii) two modified internucleoside (e.g., a phosphorothioate and/or MMI) linkages, and the linkages are between nucleotides 1 and 2, and 2 and 3;
(iii) modified internucleoside (e.g., a phosphorothioate and/or MMI) linkages between any one or more of nucleotides 1 and 2, 2 and 3, and 3 and 4;
(iv) modified internucleoside (e.g., a phosphorothioate and/or MMI) linkages between any one or more of nucleotides 1 and 2, 2 and 3, 3 and 4, and 4 and 5;
(v) modified internucleoside (e.g., a phosphorothioate and/or MMI) linkages between any one or more of nucleotides 1 and 2, 2 and 3, 3 and 4, 4 and 5, and 5 and 6;
(vi) modified internucleoside (e.g., a phosphorothioate and/or MMI) linkages between any one or more of nucleotides 1 and 2, 2 and 3, 3 and 4, 4 and 5, 5 and 6, and 6 and 7; or
(vii) modified internucleoside (e.g., a phosphorothioate and/or MMI) linkages between any one or more of nucleotides 1 and 2, 2 and 3, 3 and 4, 4 and 5, 5 and 6, 6 and 7, and 7 and 8.

64. The Cas9-IID system of claim 37, wherein the modification is at the 5′ end of the guide RNA and comprises at least one of 2′-OMe, 2′-O-MOE, inverted abasic, or 2′-F modified nucleotide.

65. The Cas9-IID system of any one of claims 1-28 or 37-64, or method of any one of claims 30-36, or engineered guide RNA of any one of claims 37-64, wherein the guide RNA comprises, in series, a 5′-N region, S1′ region, a S1″ region substantially complementary to the S1′ region, a S2′ region, a S3′ region, a S4′ region, a S4″ region substantially complementary to the S4′ region, a S5′ region, a S5″ region substantially complementary to the S5′ region, a D3″ region substantially complementary to the S3′ region, a S6′ region, a S6″ region substantially complementary to the S6′ region, a S2″ region substantially complementary to the S2′ region, and 3′-tail region, wherein each region is independently from 1 to 25 (e.g., 2, 3, 4, 5, 6, 7, 8, 9, 10, 11, 12, 13, 14, 15, 16, 17, 18, 19, 20, 21, 22, 23, 24 or 25) nucleotides in length, and wherein the regions are connected independently by via 1, 2, 3, 4, 5, 6, 7, 8, or more nucleotides, or connected directly by a modified (e.g., phosphorothioate, imidp, or MMI) or unmodified (e.g., phosphodiester) internucleoside linkage

66. An engineered guide RNA comprising, in series, a 5′-N region, S1′ region, a S1″ region substantially complementary to the S1′ region, a S2′ region, a S3 region, a S4′ region, a S4″ region substantially complementary to the S4′ region, a S5′ region, a S5′ region substantially complementary to the S5′ region, a D3″ region substantially complementary to the S3 region, a S6′ region, a S6″ region substantially complementary to the S6′ region, a S2″ region substantially complementary to the S2′ region, and 3′-tail region, wherein the guide RNA is less than 155 (e.g., 154, 153, 152, 151, 150, 149, 148, 147, 146, 145, 144, 143, 142, 141, 140, 139, 138, 137, 136, 135, 134, 133, 132, 131, 130, 129, 128, 127, 126, 125, 124, 123, 122, 121, 120, 119, 118, 117, 116, 115, 114, 113, 112, 111, 110, 109, 108, 107, 106, 105, 104, 103, 102, 101, 100 or less) nucleotides in length, and wherein: (i) 5′-N, S5′, S″, S6′ and S6″ regions are independently absent or independently from 1 to 25 (e.g., 2, 3, 4, 5, 6, 7, 8, 9, 10, 11, 12, 13, 14, 15, 16, 17, 18, 19, 20, 21, 22, 23, 24 or 25) nucleotides in length; (ii) the S1′, S1″, S2′, S2″, S3′, S3″, S4′, S4″, and the 3′-tail regions are independently from 1 to 25 (e.g., 2, 3, 4, 5, 6, 7, 8, 9, 10, 11, 12, 13, 14, 15, 16, 17, 18, 19, 20, 21, 22, 23, 24 or 25) nucleotides in length; (iii) the 5′-N and the S1′ region are connected to each other via 1, 2, 3, 4, 5, 6, 7, 8, or more nucleotides, or directly by a modified (e.g., phosphorothioate, midophosphoramidate (imidp), or methylenemethylimino (MMI, 3′-CH₂—N(CH₃)—O-5′) or unmodified (e.g., phosphodiester) internucleoside linkage; (iv) the S1′ and S1″ regions are connected to each other via 1, 2, 3, 4, 5, 6, 7, 8, or more nucleotides, or directly by a modified (e.g., phosphorothioate, midophosphoramidate (imidp), or methylenemethylimino (MMI, 3′-CH₂—N(CH₃)—O-5′) or unmodified (e.g., phosphodiester) internucleoside linkage; (v) the S2′ and S3′ regions are connected to each other via 1, 2, 3, 4, 5, 6, 7, 8, or more nucleotides, or directly by a modified (e.g., phosphorothioate, midophosphoramidate (imidp), or methylenemethylimino (MMI, 3′-CH₂—N(CH₃)—O-5′) or unmodified (e.g., phosphodiester) internucleoside linkage: (vi) S3′ and S4′ regions are connected to each other via 1, 2, 3, 4, 5, 6, 7, 8, or more nucleotides, or directly by a modified (e.g., phosphorothioate, midophosphoramidate (imidp), or methylenemethylimino (MMI, 3′-CH₂—N(CH₃)—O-5′) or unmodified (e.g., phosphodiester) internucleoside linkage; (vii) the S4′ and S4″ regions are connected to each other via 1, 2, 3, 4, 5, 6, 7, 8, or more nucleotides, or directly by a modified (e.g., phosphorothioate, midophosphoramidate (imidp), or methylenemethylimino (MMI, 3′-CH₂—N(CH₃)—O-5′) or unmodified (e.g., phosphodiester) internucleoside linkage; (viii) the S4″ and S5′ regions are connected to each other via 1, 2, 3, 4, 5, 6, 7, 8, or more nucleotides, or directly by a modified (e.g., phosphorothioate, midophosphoramidate (imidp), or methylenemethylimino (MMI, 3′-CH₂—N(CH₃)—O-5′) or unmodified (e.g., phosphodiester) internucleoside linkage; (ix) the S5′ and S5″ regions are connected to each other via 1, 2, 3, 4, 5, 6, 7, 8, or more nucleotides, or directly by a modified (e.g., phosphorothioate, midophosphoramidate (imidp), or methylenemethylimino (MMI, 3′-CH₂—N(CH₃)—O-5′) or unmodified (e.g., phosphodiester) internucleoside linkage; (x) the S5″ and S3″ regions are connected to each other via 1, 2, 3, 4, 5, 6, 7, 8, or more nucleotides, or directly by a modified (e.g., phosphorothioate, midophosphoramidate (imidp), or methylenemethylimino (MMI, 3′-CH₂—N(CH₃)—O-5′) or unmodified (e.g., phosphodiester) internucleoside linkage; (xi) the S3″ and S6′ regions are connected to each other via 1, 2, 3, 4, 5, 6, 7, 8, or more nucleotides, or directly by a modified (e.g., phosphorothioate, midophosphoramidate (imidp), or methylenemethylimino (MMI, 3′-CH₂—N(CH₃)—O-5′) or unmodified (e.g., phosphodiester) internucleoside linkage; (xii) the S6′ and S6″ regions are connected to each other via 1, 2, 3, 4, 5, 6, 7, 8, or more nucleotides, or directly by a modified (e.g., phosphorothioate, midophosphoramidate (imidp), or methylenemethylimino (MMI, 3′-CH₂—N(CH₃)—O-5′) or unmodified (e.g., phosphodiester) internucleoside linkage; (xii2″ i) the S6″ and the S2″ regions are connected to each other via 1, 2, 3, 4, 5, 6, 7, 8, or more nucleotides, or directly by a modified (e.g., phosphorothioate, midophosphoramidate (imidp), or methylenemethylimino (MMJ, 3′-CH₂—N(CH₃)—O-5′) or unmodified (e.g., phosphodiester) internucleoside linkage; (xiv) the S2′ and the 3′-tail regions are connected to each other via 1, 2, 3, 4, 5, 6, 7, 8, or more nucleotides, or directly by a modified (e.g., phosphorothioate, midophosphoramidate (imidp), or methylenemethylimino (MMI, 3′-CH₂—N(CH₃)—O-5′) or unmodified (e.g., phosphodiester) internucleoside linkage, and optionally, the guide RNA does not comprise the nucleotide sequence of SEQ ID NO: 409.

67. The engineered guide RNA of claim 66, wherein the guide nucleic acid comprises at least one nucleic acid modification.

68. The engineered guide RNA of claim 66 or 67, wherein the guide nucleic acid comprises at least one nucleic acid modification selected from the group consisting of nucleobase modifications (e.g., a non-natural or modified nucleobase), sugar modifications, inter-sugar linkage modifications (e.g., modified internucleotide linkages), conjugates (e.g., ligands), and any combinations thereof. Nucleic acid modifications also include unnatural, or degenerate nucleobases.

69. The engineered guide RNA of any one of claims 66-68, wherein the guide RNA comprises a modified nucleotide selected from the group consisting of 2′-O-methyl (2′-OMe) nucleotides, 2′-fluoro (2′-F) nucleotides, bridged nucleic acid (BNA) nucleotides (e.g., 2′-O.4′-C-methylene (locked nucleic acid, LNA) nucleotides, 2′-O,4′-C-ethylene (locked nucleic acid, ENA) nucleotides, 5′-methyl-BNA, cEt BNA, cMOE BNA, oxy amino BNA and vinyl-carbo BNA), anhydrohexitol (1,5-anhydrohexitol nucleic acid, HNA) nucleotides, cyclohexene (Cyclohexene nucleic acid, CeNA) nucleotides, 2′-methoxyethyl (2′-MOE) nucleotides, 2′-O-allyl nucleotides, 2′-C-allyl ribose nucleotides, 2′-O—N-methylacetamido (2′-O-NMA) nucleotides, a 2′-O-dimethylaminoethoxyethyl (2′-O-DMAEOE) nucleotides, 2′-O-aminopropyl (2′-O-AP) nucleotides, 2′-F arabinose (2′-ara-F) nucleotides, threose (Threose nucleic acid, TNA) nucleotides, and acyclic nucleotides (e.g., unlocked nucleic acids (UNA) and 2,3-dihydroxylpropyl (glycol nucleic acid, GNA)): a modified internucleoside linkage: a non-natural or modified nucleobase; or a combination thereof, optionally, the modified nucleotide is a 2′-O-methyl (2′-OMe) nucleotide or a 2′-fluoro nucleotide, and/or a nucleotide comprising a non-natural or modified nucleobase.

70. The engineered guide RNA of any one of claims 66-69, wherein the guide RNA comprises at least one (e.g., 1, 2, 3, 4, 5, or more) modified internucleoside linkage (e.g., a modified internucleoside linkage selected independently from the group consisting of phosphorothioates (R, S, or racemic), phosphorodithioates, methylenemethylimino (MMI, 3′-CH₂—N(CH₃)—O-5′), phosphotriesters, alkylphosphonates (e.g., methylphosphonates), phosphoramidate, methylenemethylimino (—CH₂—N(CH₃)—O—CH₂—), thiodiester (—O—C(O)—S—), thionocarbamate (—O—C(O)(NH)—S—), siloxane (—O—Si(H)₂—O— and dialkylsiloxane), N,N′-dimethylhydrazine (—CH₂—N(CH₃)—N(CH₃)—), amide-3 (3′-CH₂—C(═O)—N(H)-5′), amide-4 (3′-CH₂—N(H)—C(═O)-5′)), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methykenecarbonylamino, methylenehydrazo, methylenedimethylhydrazo, methyleneoxymethylimino, ethers (C3′-O—C5′), thioethers (C3′-S—C5′), thioacetamido (C3*—N(H)—C(═O)—CH₂—S—C5*, C3′-O—P(O)—O—SS—C5′), C3′-CH₂—NH—NH—C5′, 3′-NHP(O)(OCH₃)—O-5′, 3′-NHP(O)(OCH₃)—O-5′), 2′->5′ internucleoside linkages, 2′->3′ internucleoside linkages, 3′->3′ internucleoside linkages, and 5′->5′ internucleoside linkages, optionally the modified internucleoside linkage is phosphorothioate, imidp or MMI, more optionally the modified internucleoside linkage is phosphorothioate (PS).

71. The engineered guide RNA of any one of claims 66-70, wherein the guide RNA comprises at least one duplex stabilizing modification.

72. The engineered guide RNA of any one of claims 66-71, wherein the guide RNA comprises at least one duplex stabilizing modification selected from the group consisting of 2′-F nucleotides, 2′-OMe nucleotides, 2′-methoxyethyl nucleotides, 2,6-diaminopurine nucleotides, 5-methyl cytidine, N4-ethyl cytidine, 5-propynyl cytidine, 5-propynyl uridine, 5-hydroxybutynl-2′-deoxyuridine, 8-aza-7-deazaguanosine, a locked nucleic acid (LNA), and/or covalent cross-linking of two strands in the duplex.

73. The engineered guide RNA of any one of claims 66-72, wherein the 3′-tail region comprises any one of: (i) a modification of any one or more of the last 7, 6, 5, 4, 3, 2, or 1 nucleotides; (ii) one modified nucleotide: (ii) two modified nucleotides; (iii) three modified nucleotides; (iv) four modified nucleotides; (v) five modified nucleotides; (vi) six modified nucleotides; or (vii) seven modified nucleotides.

74. The engineered guide RNA of any one of claims 66-72, wherein, the 3′-tail region comprises: (i) a modified internucleoside linkage (e.g., PS, imidp or MMI linkage) between nucleotides; (ii) a 2′-OMe nucleotide; (iii) a 2′-O-MOE nucleotide; (iv) a 2′-F nucleotide; (v) a LNA nucleotide; (vi) a 3′->3′ linkage between nucleotides; (vii) an inverted abasic nucleotide; or (viii) a combination of one or more of (i)-(vii).

75. The engineered guide RNA of any one of claims 66-72, wherein the 3′-tail region comprises: (i) a modified internucleoside linkage (e.g., PS, imidp or MMI linkage) between the last and second to last nucleotides; (ii) a modified internucleoside linkage (e.g., PS, imidp or MMI linkage) between the last and second to last nucleotide and a PS linkage between the second and third to last nucleotides; (iii) modified internucleoside linkages (e.g., PS and/or MMI linkage) between any one or more of the last four nucleotides; (iv) modified internucleoside linkages (e.g., PS and/or MMI linkage) between any one or more of the last five nucleotides; and/or or (v) modified internucleoside linkages (e.g., PS and/or MMI linkage) between any one or more of the last 2, 3, 4, 5, 6, 7, 8, 9, or 10 nucleotides.

76. The engineered guide RNA of any one of claims 66-72, wherein the 3′-tail region comprises: (i) a modification of one or more of the last 1-7 nucleotides, wherein the modification is a modified internucleoside linkages (e.g., PS and/or MMI linkage), inverted abasic nucleotide, a 3′->3′ internucleoside linkage, 2′-OMe, 2′-O-MOE, 2′-F, LNA, or a combination thereof: (ii) a modification to the last nucleotide with 2′-OMe, 2′-O-MOE, 2′-F, LNA, or combinations thereof, and an optional one or two modified internucleoside linkages (e.g., PS and/or MMI linkage) to the next nucleotide; (iii) a modification to the last and/or second to last nucleotide with 2′-OMe, 2′-O-MOE, 2′-F, LNA, or combinations thereof, and optionally one or more modified internucleoside linkages (e.g., PS and/or MMI linkage); iv. a modification to the last, second to last, and/or third to last nucleotides with 2′-OMe, 2′-O-moe, 2′-F, or combinations thereof, and optionally one or more modified internucleoside linkages (e.g., PS and/or MMI linkage): (v) a modification to the last, second to last, third to last, and/or fourth to last nucleotides with 2′-OMe, 2′-O-MOE, 2′-F, LNA or combinations thereof, and optionally one or more modified internucleoside linkages (e.g., PS and/or MMI linkage); and/or (vi) a modification to the last, second to last, third to last, fourth to last, and/or fifth to last nucleotides with 2′-OMe, 2′-O-MOE, 2′-F, LNA, or combinations thereof, and optionally one or more modified internucleoside linkages (e.g., PS and/or MMI linkage).

77. The engineered guide RNA of any one of claims 66-72, wherein the 3′-tail region comprises: (i) a 2′-OMe modified nucleotide at the lastposition, three consecutive 2′-O-MOE modified nucleotides immediately 5′ to the 2′-OMe modified nucleotide, and three consecutive PS linkages between the last three nucleotides: (ii) five consecutive 2′-OMe modified nucleotides from the 3′ end of the 3′ terminus, and three PS linkages between the last three nucleotides: (iii) an inverted abasic modified nucleotide at the last position; (iv) an inverted abasic modified nucleotide at the last position, and three consecutive 2′-OMe modified nucleotides at the last three positions; (v) 15 consecutive 2′-OMe modified nucleotides from the 3′ end, five consecutive 2′-F modified nucleotides immediately 5′ to the 2′-OMe modified nucleotides, and three PS linkages between the last three nucleotides; (vi) alternating 2′-OMe modified nucleotides and 2′-F modified nucleotides at the last 20 nucleotides, and three PS linkages between the last three nucleotides; (vii) two or three consecutive 2′-OMe modified nucleotides, and three PS linkages between the last three nucleotides; (viii) one PS linkage between the last and next to last nucleotides; and/or (ix) 15 or 20 consecutive 2′-OMe modified nucleotides, and three PS linkages between the last three nucleotides.

78. The engineered guide RNA of any one of claims 66-77, wherein the guide RNA comprises, at its 5′-end, any one of: (i) one modified nucleotide; (ii) two modified nucleotides; (iii) three modified nucleotides; (iv) four modified nucleotides; (v) five modified nucleotides; (vi) six modified nucleotides; or (vii) seven modified nucleotides, optionally wherein the guide RNA comprises, at its 5′-end, between 1 and 7, between 1 and 5, between 1 and 4, between 1 and 3, or between 1 and 2 modified nucleotides.

79. The engineered guide RNA of any one of claims 66-77, wherein the, the guide RNA comprises, at its 5′-end, one or more of: (i) a modified internucleoside linkage (e.g., a phosphorothioate and/or MMI linkage) between nucleotides; (ii) a 2′-OMe nucleotide; (iii) a 2′-O-MOE nucleotide: (iv) a 2′-F nucleotide; (v) a LNA; (vi) a 3′->3′ linkage; (vii) an inverted abasic modified nucleotide; (viii) a deoxyribonucleotide; (ix) an inosine; and (x) combinations of one or more of (i)-(ix).

80. The engineered guide RNA of any one of claims 66-77, wherein the guide RNA comprises, at its 5′-end, about 1-2, 1-3, 1-4, 1-5, 1-6, or 1-7 modified internucleoside (e.g., a phosphorothioate and/or MMI) linkages between nucleotides, optionally wherein the guide RNA comprises, at its 5′-end, 1, 2, 3, 4, 5, 6, and/or 7 modified internucleoside (e.g., a phosphorothioate and/or MMI) linkages between nucleotides. In some embodiments, the guide RNA comprises, at its 5-end, any one of: (i) one modified internucleoside (e.g., a phosphorothioate and/or MMI) linkage, and the linkage is between nucleotides 1 and 2; (ii) two modified internucleoside (e.g., a phosphorothioate and/or MMI) linkages, and the linkages are between nucleotides 1 and 2, and 2 and 3; (iii) modified internucleoside (e.g., a phosphorothioate and/or MMI) linkages between any one or more of nucleotides 1 and 2, 2 and 3, and 3 and 4; (iv) modified internucleoside (e.g., a phosphorothioate and/or MMI) linkages between any one or more of nucleotides 1 and 2, 2 and 3, 3 and 4, and 4 and 5: (v) modified internucleoside (e.g., a phosphorothioate and/or MMI) linkages between any one or more of nucleotides 1 and 2, 2 and 3, 3 and 4, 4 and 5, and 5 and 6; (vi) modified internucleoside (e.g., a phosphorothioate and/or MMI) linkages between any one or more of nucleotides 1 and 2, 2 and 3, 3 and 4, 4 and 5, 5 and 6, and 6 and 7; or (vii) modified internucleoside (e.g., a phosphorothioate and/or MMI) linkages between any one or more of nucleotides 1 and 2, 2 and 3, 3 and 4, 4 and 5, 5 and 6, 6 and 7, and 7 and 8.

81. The engineered guide RNA of any one of claims 66-77, wherein the, the guide RNA comprises, at its 5-end, at least one 2′-OMe, 2′-O-MOE, inverted abasic, or 2′-F modified nucleotide.

82. The engineered guide RNA of any one of claims 66-81, wherein the S1′ and S1″ regions together form a double stranded structure (duplex region), optionally the duplex comprises an internal loop comprising 2 to 10 (e.g., 2, 3, 4, 5, 6, 7, 8 or 9) nucleotides, and/or a bulge loop comprising 1 to 10 (e.g., 2, 3, 4, 5, 6, 7, 8 or 9), more optionally, the duplex comprises an internal loop comprising 2 to 10 (e.g., 2, 3, 4, 5, 6, 7, 8 or 9) nucleotides.

83. The engineered guide RNA of any one of claims 66-82, wherein the S2′ and S2″ regions together form a double stranded structure), optionally the duplex comprises an internal loop comprising 2 to 10 (e.g., 2, 3, 4, 5, 6, 7, 8 or 9) nucleotides, and/or a bulge loop comprising 1 to 10 (e.g., 2, 3, 4, 5, 6, 7, 8 or 9) nucleotides, preferably, the duplex does not comprise a bulge or internal loop.

84. The engineered guide RNA of any one of claims 66-83, wherein the S3′ and S3″ regions together form a double stranded structure), optionally the duplex comprises an internal loop comprising 2 to 10 (e.g., 2, 3, 4, 5, 6, 7, 8 or 9) nucleotides, and/or a bulge loop comprising 1 to 10 (e.g., 2, 3, 4, 5, 6, 7, 8 or 9) nucleotides, more optionally, the duplex does not comprise a bulge or internal loop, or the duplex region comprises a 1 nucleotide bulge, preferably, the duplex does not comprise a bulge or internal loop.

85. The engineered guide RNA of any one of claims 66-84, wherein the S4′ and S4″ regions together form a double stranded structure), optionally the duplex comprises an internal loop comprising 2 to 10 (e.g., 2, 3, 4, 5, 6, 7, 8 or 9) nucleotides, and/or a bulge loop comprising 1 to 10 (e.g., 2, 3, 4, 5, 6, 7, 8 or 9) nucleotides, optionally, the duplex comprises an internal loop comprising 2 to 10 (e.g., 2, 3, 4, 5, 6, 7, 8 or 9) nucleotides.

86. The engineered guide RNA of any one of claims 66-83, wherein the S5′ and S5″ regions together form a double stranded structure, optionally the duplex comprises an internal loop comprising 2 to 10 (e.g., 2, 3, 4, 5, 6, 7, 8 or 9) nucleotides, and/or a bulge loop comprising 1 to 10 (e.g., 2, 3, 4, 5, 6, 7, 8 or 9) nucleotides, preferably, the duplex comprises an internal loop comprising 2 to 10 (e.g., 2, 3, 4, 5, 6, 7, 8 or 9) nucleotides.

87. The engineered guide RNA of any one of claims 66-86, wherein the S6′ and S6″ region regions together form a double stranded structure, optionally the duplex comprises an internal loop comprising 2 to 10 (e.g., 2, 3, 4, 5, 6, 7, 8 or 9) nucleotides, and/or a bulge loop comprising 1 to 10 (e.g., 2, 3, 4, 5, 6, 7, 8 or 9) nucleotides, preferably, the duplex does not comprises an internal loop.

88. The engineered guide RNA of any one of claims 66-87, wherein the S5′ and S5″ regions are absent.

89. The engineered guide RNA of any one of claims 66-88, wherein the S 6′ and S6″ regions are absent.

90. The engineered guide RNA of any one of claims 66-89, wherein the S5′, S5″, S6′ and S6″ regions are absent.

91. The engineered guide RNA of any one of claims 66-90, wherein the guide RNA is at least 1 (e.g., 2, 3, 4, 5, 6, 7, 8, 9, 10, 11, 12, 13, 14, 15, 16, 17, 18, 19, 20 or more) nucleotides shorter than SEQ ID NO: 409, optionally, the guide RNA is at least 5, 10, 15, 20, 25 or more nucleotides shorter than SEQ ID NO: 409.

92. The engineered guide RNA of any one of claims 66-91, wherein the guide RNA comprises a nucleotide sequence having at least 60%, 65%, 70%, 75%, 80%, 85%, 90%, 95% identity to a nucleic acid selected from the group consisting of SEQ ID NOs: 315-408, 410-506, 509-756 or 759-772.

93. The Cas9-IID system of any one of claims 1-28 or 37-64, wherein the guide RNA is an engineered guide RNA of any one of claims 66-92.
